(19) United States
(12) Patent Application Publication
(10) Pub. No.: US 2003/0215805 A1
(43) Pub. Date: Nov. 20, 2003

(54) Novel genes, compositions, kits, and methods for identification, assessment prevention, and therapy of breast cancer

(75) Inventors: James Lillie, Natick, MA (US); Adam Palermo, Stanford, MA (US); Youzhen Wang, Newton, MA (US); Kathleen Steinmann, Winchester, MA (US); Josh Elias, Brookline, MA (US); Maureen Mertens, Stow, MA (US)

Correspondence Address: LAHIVE & COCKFIELD, 28 STATE STREET, BOSTON, MA, 02109, US

(73) Assignee: Millennium Pharmaceutical, Inc., Cambridge, MA (US)

(21) Appl. No.: 10/125,968

(22) Filed: Apr. 19, 2002

Related U.S. Application Data
(60) Provisional application No. 60/285,163, filed on Apr. 20, 2001.

(51) Int. Cl. C12Q 1/68; G01N 33/574; G06F 19/00; G01N 33/48; G01N 33/50

(52) U.S. Cl. 435/6; 435/7.23; 702/19

(57) ABSTRACT

The invention relates to compositions, kits, and methods for detecting, characterizing, preventing, and treating human breast cancers. A variety of marker genes are provided, wherein changes in the levels of expression of one or more of the marker genes is correlated with the presence of breast cancer.

### RELATED APPLICATIONS

The present application claims priority to U.S. provisional patent application serial No. 60/285,163, filed on Apr. 20, 2001, which is expressly incorporated by reference.

### FIELD OF THE INVENTION

The field of the invention is breast cancer, including diagnosis, characterization, management, and therapy of breast cancer.

### BACKGROUND OF THE INVENTION

The increased number of cancer cases reported in the United States, and, indeed, around the world, is a major concern. Currently there are only a handful of treatments available for specific types of cancer, and these provide no absolute guarantee of success. In order to be most effective, these treatments require not only an early detection of the malignancy, but a reliable assessment of the severity of the malignancy.

The incidence of breast cancer, a leading cause of death in women, has been gradually increasing in the United States over the last thirty years. In 1997, it was estimated that 181,000 new cases were reported in the U.S., and that 44,000 people would die of breast cancer (Parker et al, 1997, CA Cancer J. Clin. 47:5-27; Chu et al, 1996, J. Nat. Cancer Inst. 88:1571-1579). While the pathogenesis of breast cancer is unclear, transformation of normal breast epithelium to a malignant phenotype may be the result of genetic factors, especially in women under 30 (Miki et al., 1994, Science, 266:66-71). The discovery and characterization of BRCA1 and BRCA2 has recently expanded our knowledge of genetic factors which can contribute to familial breast cancer. Germ-line mutations within these two loci are associated with a 50 to 85% lifetime risk of breast and/or ovarian cancer (Casey, 1997, Curr. Opin. Oncol. 9:88-93; Marcus et al, 1996, Cancer 77:697-709). However, it is likely that other, non-genetic factors also have a significant effect on the etiology of the disease. Regardless of its origin, breast cancer morbidity and mortality increases significantly if it is not detected early in its progression. Thus, considerable effort has focused on the early detection of cellular transformation and tumor formation in breast tissue.

Currently, the principal manner of identifying breast cancer is through detection of the presence of dense tumorous tissue. This may be accomplished to varying degrees of effectiveness by direct examination of the outside of the breast, or through mammography or other X-ray imaging methods (Jatoi, 1999, Am. J. Surg. 177:518-524). The latter approach is not without considerable cost, however. Every time a mammogram is taken, the patient incurs a small risk of having a breast tumor induced by the ionizing properties of the radiation used during the test. In addition, the process is expensive and the subjective interpretations of a technician can lead to imprecision, e.g., one study showed major clinical disagreements for about one-third of a set of mammograms that were interpreted individually by a surveyed group of radiologists. Moreover, many women find that undergoing a mammogram is a painful experience. Accordingly, the National Cancer Institute has not recommended mammograms for women under fifty years of age, since this group is not as likely to develop breast cancers as are older women. It is compelling to note, however, that while only about 22% of breast cancers occur in women under fifty, data suggests that breast cancer is more aggressive in pre-menopausal women.

It would therefore be beneficial to provide specific methods and reagents for the diagnosis, staging, prognosis, monitoring, and treatment of diseases associated with breast cancer, or to indicate a predisposition to such for preventative measures.

### SUMMARY OF THE INVENTION

The invention relates to novel genes associated with breast cancer as well as methods of assessing whether a patient is afflicted with breast cancer. The methods of the present invention comprise the step of comparing the level of expression of a marker in a patient sample, wherein the marker is listed in Table 1 and the normal level of expression of the marker in a control, e.g., a sample from a patient without breast cancer. A significant difference between the level of expression of the marker in the patient sample and the normal level is an indication that the patient is afflicted with breast cancer. Preferably, a protein corresponding to the marker is a secreted protein or is predicted to correspond to a secreted protein. Alternatively, the marker can correspond to a protein having an extracellular portion, to one which is normally expressed in breast tissue at a detectable level, or both.

In one method, the marker(s) are preferably selected such that the positive predictive value of the method is at least about 10%. Also preferred are embodiments of the method wherein the marker is over- or under-expressed by at least two-fold in at least about 20% of stage 0 breast cancer patients, stage I breast cancer patients, stage IIA breast cancer patients, stage IIB breast cancer patients, stage IIIA breast cancer patients, stage IIIB breast cancer patients, stage IV breast cancer patients, grade I breast cancer patients, grade II breast cancer patients, grade III breast cancer patients, malignant breast cancer patients, ductal carcinoma breast cancer patients, and lobular carcinoma breast cancer patients.

In one embodiment of the methods of the present invention, the patient sample is a breast tissue-associated body fluid. Such fluids include, for example, blood fluids, lymph and cystic fluids, as well as nipple aspirates. In another embodiment, the sample comprises cells obtained from the patient. In another embodiment, the patient sample is in vivo.

In accordance with the methods of the present invention, the level of expression of a marker gene in a sample can be assessed, for example, by detecting the level in the sample of:

a protein encoded by the marker gene, or a polypeptide or a fragment comprising the protein (e.g. using a reagent, such as an antibody, an antibody derivative, or a single chain antibody, which binds specifically with the protein or a fragment thereof);

a metabolite which is produced directly (i.e., catalyzed) or indirectly by the protein encoded by the marker gene; and/or

a polynucleotide (e.g. an mRNA, hnRNA, cDNA) produced by or derived from the expression of the marker gene or a fragment of the polynucleotide (e.g. by contacting polynucleotides obtained or derived from the sample with a substrate having affixed thereto a nucleic acid comprising the marker gene sequence or a portion of such sequence).

The methods of the present invention are useful for further diagnosing patients having an identified breast mass or symptoms associated with breast cancer. The methods of the present invention may therefore be used to diagnose breast cancer or its precursors. The methods of the present invention can further be of particular use with patients having an enhanced risk of developing breast cancer (e.g., patients having a familial history of breast cancer and patients identified as having a mutant oncogene) in providing early detection of breast cancer. The methods of the present invention may further be of particular use in monitoring the efficacy of treatment of a breast cancer patient (e.g. the efficacy of chemotherapy).

The methods of the present invention may be performed by assessing the expression of a plurality (e.g. 2, 3, 5, or 10 or more) of breast cancer marker genes. According to a method involving a plurality of marker genes, the level of expression in a patient sample of each of a plurality of marker genes, including at least one that is selected from the marker genes listed in Table 1, is compared with the normal level of expression of each of the plurality of marker genes in samples of the same type obtained from control subjects, i.e., human subjects not afflicted with breast cancer. A significantly altered, preferably increased, level of expression in the patient sample of one or more of the marker genes, or some combination thereof, relative to those marker genes' expression levels in samples from control subjects, is an indication that the patient is afflicted with or has a higher than normal risk for developing breast cancer. The methods of the present invention may be practiced using one or more marker genes of the invention in combination with one or more known breast cancer marker genes.

In a preferred method of assessing whether a patient is afflicted with breast cancer (e.g., new detection (“screening”), detection of recurrence, reflex testing), the method comprises comparing:

a) the level of expression of one or several breast cancer marker genes, in a patient sample, wherein at least one such gene is selected from the marker genes listed in Table 1, and

b) the normal level of expression of the same marker gene(s) in a sample from a control subject having no breast cancer.

A significantly altered expression of one or several marker genes in the patient sample relative to the normal expression levels in the sample from the control subject is an indication that the patient is afflicted with breast cancer. In preferred embodiments, a significantly increased expression of one or more marker genes in the patient sample relative to the normal expression levels in the sample from the control subject is an indication that the patient is afflicted with breast cancer.

The invention further relates to a method of assessing the efficacy of a therapy for inhibiting breast cancer in a patient. This method comprises comparing:

a) expression of one or several breast cancer marker genes in a first sample obtained from the patient prior to providing at least a portion of the therapy to the patient, wherein at least one such marker gene is selected from the marker genes listed in Table 1, and

b) expression of the same marker gene(s) in a second sample obtained from the patient following provision of the portion of the therapy.

A significantly altered expression of the level of expression of one or several of the marker genes in the second sample, relative to the first sample, is an indication that the therapy is efficacious for inhibiting breast cancer in the patient. In preferred embodiments, a significantly reduced expression of one or several of the marker genes in the second sample, relative to the first sample, is an indication that the therapy is efficacious.

It will be appreciated that in this method the “therapy” may be any therapy for treating breast cancer including, but not limited to, chemotherapy, immunotherapy, gene therapy, radiation therapy and surgical removal of tissue. Thus, the methods of the invention may be used to evaluate a patient before, during and after therapy, for example, to evaluate the reduction in tumor burden.

The present invention therefore further comprises a method for monitoring the progression of breast cancer in a patient, the method comprising:

a) detecting in a patient sample at a first time point, the expression of one or several breast cancer marker genes, wherein at least one such marker gene is selected from the marker genes listed in Table 1;

b) repeating step a) with patient sample obtained at a subsequent point in time; and

c) comparing the level of expression detected in steps a) and b), and therefrom monitoring the progression of breast cancer in the patient.

A significantly altered level of expression of one or several of the marker genes in the subsequent point in time, relative to the level of expression at the first time point, is an indication that the breast cancer has progressed. In preferred embodiments, a significantly increased expression of one or several of the marker genes in the subsequent point in time, relative to the first time point, is an indication that the breast cancer has progressed. Conversely, a significantly decreased expression of one or several of the marker genes in the subsequent point in time is an indication that the breast cancer has regressed.

The present invention also includes a method for assessing the aggressiveness of breast cancer, the method comprising comparing:

a) the level of expression of one or several breast cancer marker genes in a patient sample, wherein at least one such marker gene is selected from the marker genes listed in Table 1, and

b) the level of expression of the same marker gene(s) in a sample from a control subject having breast cancer which is indolent.

A significantly altered level of expression of one or several of the marker genes in the patient sample, relative to the level in the control subject sample, is an indication that the patient is afflicted with an aggressive breast cancer. In preferred embodiments, a significantly increased expression of one or more marker genes in the patient sample, relative to the expression level in the control subject sample, is an indication that the patient is afflicted with an aggressive breast cancer.

The present invention also includes a method for assessing the indolence of breast cancer, the method comprising comparing:

a) the level of expression of one or several breast cancer marker genes in a patient sample, wherein at least one such marker gene is selected from the marker genes listed in Table 1, and

b) the level of expression of the same marker gene(s) in a sample from a control subject having an aggressive breast cancer.

A significantly altered level of expression of one or several of the marker genes in the patient sample, relative to the level in the control subject sample, is an indication that the patient is afflicted with an indolent breast cancer. In preferred embodiments, a significantly decreased expression of one or more marker genes in the patient sample, relative to the expression level in the control subject sample, is an indication that the patient is afflicted with an indolent breast cancer.

The present invention further includes a method for determining whether breast cancer has metastasized or is likely to metastasize in the future, the method comprising comparing:

a) the level of expression of one or several breast cancer marker genes in a patent sample, wherein at least one such marker gene is selected from the marker genes of Table 1 and

b) the level of expression of the same marker gene(s) in a sample from a control subject having non-metastasized breast cancer.

A significantly altered level of expression in the patient sample, relative to level of expression in the control subject sample, is an indication that the patient is afflicted with breast cancer that has metastasized or is likely to metastasize in the future. In preferred embodiments, a significantly increased expression of one or more marker genes in the patient sample, relative to the expression level in the control subject sample, is an indication that the patient is afflicted with breast cancer that has metastasized or is likely to metastasize in the future.

The present invention also includes a method for determining whether breast cancer has not metastasized or is not likely to metastasize in the future, the method comprising comparing:

a) the level of expression of one or several breast cancer marker genes in a patent sample, wherein at least one such marker gene is selected from the marker genes of Table 1 and

b) the level of expression of the same marker gene(s) in a sample from a control subject having metastasized breast cancer.

A significantly altered level of expression in the patient sample, relative to the level of expression in the control subject sample, is an indication that the patient is afflicted with breast cancer that has not metastasized or is not likely to metastasize in the future. In preferred embodiments, a significantly decreased expression of one or more marker genes in the patient sample, relative to the expression level in the control subject sample, is an indication that the patient is afflicted with breast cancer that has not metastasized or is not likely to metastasize in the future.

The invention also includes a method of selecting a composition for inhibiting breast cancer in a patient. This method comprises the steps of:

a) obtaining a sample comprising cancer cells from the patient;

b) separately maintaining aliquots of the sample in the presence of a plurality of test compositions;

c) comparing expression of one or more breast cancer marker genes, including at least one from the marker genes listed within Table 1, in each of the aliquots; and

d) selecting one of the test compositions which alters the level of expression of one or more of the marker genes in the aliquot containing that test composition, relative to other test compositions.

In preferred embodiments, the test composition which significantly reduces the expression of one or more marker genes, relative to the expression in the presence of another test composition, is selected.

In addition, the invention includes a method of inhibiting breast cancer in a patient. This method comprises the steps of:

a) obtaining a sample comprising cancer cells from the patient;

b) separately maintaining aliquots of the sample in the presence of a plurality of test compositions;

c) comparing expression of one or several breast cancer marker genes, including at least one marker genes listed within Table 1, in each of the aliquots; and

d) administering to the patient at least one of the test compositions which significantly alters the level of expression of the marker gene in the aliquot containing that test composition, relative to other test compositions.

In preferred embodiments, the test composition which significantly reduces the expression of one or more marker genes, relative to the expression in the presence of another test composition, is administered to the patient.

The invention also includes a kit for assessing whether a patient is afflicted with breast cancer or its precursors. This kit comprises reagents for assessing expression of one or several breast cancer marker genes, including at least one of the marker genes listed within Table 1.

In another aspect, the invention relates to a kit for assessing the suitability of each of a plurality of compounds for inhibiting a breast cancer in a patient. The kit comprises a reagent for assessing expression of one or several breast cancer marker genes, including at least one of the marker genes listed in Table 1, and may also comprise a plurality of compounds.

In another aspect, the invention relates to a kit for assessing the presence of breast cancer cells. This kit comprises an antibody which binds specifically with a protein encoded by one of the marker genes listed in Table 1 or a polypeptide or a protein fragment comprising the protein. The kit may also comprise a plurality of antibodies, wherein the plurality binds specifically with a protein encoded by one of the marker genes listed in Table 1, a polypeptide or a protein fragment comprising the protein.

The invention also includes a kit for assessing the presence of breast cancer cells, wherein the kit comprises a nucleic acid probe. The probe binds specifically with a transcribed polynucleotide encoded by one of the marker genes listed within Table 1. The kit may also comprise a plurality of nucleic acid probes, wherein each of the probes binds specifically with a transcribed polynucleotide encoded by several different breast cancer marker genes, including at least one of the marker genes listed within Table 1.

The invention further relates to a method of making an isolated hybridoma which produces an antibody useful for assessing whether a patient is afflicted with breast cancer. The method comprises immunizing a mammal with a composition comprising a protein encoded by a marker gene listed within Table 1, or a polypeptide or a protein fragment comprising the protein; isolating splenocytes from the immunized mammal; fusing the isolated splenocytes with an immortalized cell line to form hybridomas; and screening individual hybridomas for production of an antibody which specifically binds with the protein or parts thereof; to isolate the hybridoma. The invention also includes an antibody produced by this method.

The invention further includes a method of assessing the carcinogenic potential of a test compound. This method comprises the steps of:

a) maintaining separate aliquots of breast cells in the presence and absence of the test compound; and

b) comparing expression of one or several breast cancer marker genes, including at least one of the marker genes of Table 1 in each of the aliquots.

A significantly altered level of expression of one or more of the marker genes in the aliquot maintained in the presence of (or exposed to) the test compound, relative to the level of expression in the aliquot maintained in the absence of the test compound, is an indication that the test compound possesses breast carcinogenic potential. In preferred embodiments, a significantly increased expression of one of more of the marker genes in the aliquot maintained in the presence of (or exposed to) the test compound, relative to the level of expression in the aliquot maintained in the absence of the test compound, is an indication that the test compound possesses breast carcinogenic potential.

Additionally, the invention includes a kit for assessing the breast carcinogenic potential of a test compound. The kit comprises a reagent for assessing expression of a breast cancer marker gene of Table 1 in each of the aliquots.

The invention further relates to a method of treating a patient afflicted with breast cancer and/or inhibiting breast cancer in a patient at risk for developing breast cancer. This method comprises inhibiting expression (or overexpression) of a breast cancer marker gene listing within Table 1, which is overexpressed in breast cancer.

It will be appreciated that the methods and kits of the present invention may also include known cancer marker genes including known breast cancer marker genes. It will further be appreciated that the methods and kits may be used to identify cancers other than breast cancer.

### DETAILED DESCRIPTION OF THE INVENTION

The invention relates to newly discovered correlations between expression of certain marker genes and the cancerous state of breast cells. It has been discovered that the level of expression of individual marker genes and combinations of marker genes described herein correlates with the presence of breast cancer or a pre-malignant condition in a patient. Methods are provided for detecting the presence of breast cancer in a sample, the absence of breast cancer in a sample, the stage of a breast cancer, the metastatic potential of a breast cancer, the indolence or aggressiveness of the cancer, and other characteristics of breast cancer that are relevant to prevention, diagnosis, characterization and therapy of breast cancer in a patient.

Definitions

As used herein and the claims, each of the following terms has the meaning associated with it in this section.

The articles “a” and “an” are used herein to refer to one or to more than one (i.e. to at least one) of the grammatical object of the article. By way of example, “an element” means one element or more than one element.

The term “marker polynucleotide” is meant to include nucleotide transcript (hnRNA or mRNA) encoded by a breast cancer marker gene, preferably a marker gene listed in Table 1, or cDNA derived from the nucleotide transcript, or a segment of said transcript or cDNA.

The term “marker protein” is meant to include protein or polypeptide encoded by a breast cancer marker gene, preferably a marker gene listed in Table 1, or a polypeptide or protein fragment comprising said marker protein.

The term “gene product” is meant to include marker polynucleotide and marker protein encoded by the referenced gene.

As used herein the term “polynucleotide” is synonymous with “nucleic acid.” Further a polynucleotide “corresponds to” another (a first) polynucleotide if it is related to the first polynucleotide by any of the following relationships: the second polynucleotide comprises the first polynucleotide and the second polynucleotide encodes a gene product; the second polynucleotide is the complement of the first polynucleotide and, the second polynucleotide is 5′ or 3′ to the first polynucleotide in cDNA, RNA, genomic DNA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene coding for a gene product. The second polynucleotide may be an exon of the gene while the first polynucleotide may be an intron of the gene. The term “probe” refers to any molecule which is capable of selectively binding to a specifically intended target molecule, for example a marker gene of the invention. Probes can either be synthesized by one skilled in the art, or derived from appropriate biological preparations. For purposes of detection of the target molecule, probes may be specifically designed to be labeled, as described herein. Examples of molecules that can be utilized as probes include, but are not limited to, proteins, antibodies, organic monomers, RNA, DNA, and cDNA.

A “breast-associated” body fluid is a fluid which, when in the body of a patient, contacts or passes through breast cells or into which cells, nucleic acids or proteins shed from breast cells are capable of passing. Exemplary breast-associated body fluids include blood fluids, lymph, cystic fluid, urine and nipple aspirates.

The “normal” level of expression of a marker gene is the level of expression of the marker gene in breast cells or breast-associated body fluids of a subject, e.g. a human, not afflicted with breast cancer.

“Over-expression” and “under-expression” of a marker gene refer to expression of the marker gene of a patient at a greater or lesser level, respectively, than normal level of expression of the marker gene (e.g. at least two-fold greater or lesser level).

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” is a polynucleotide (e.g an RNA, a cDNA, or an analog of one of an RNA or cDNA) which is complementary to or homologous with all or a portion of a mature RNA made by transcription of a gene, such as any of the marker genes of the invention, and normal post-transcriptional processing (e.g. splicing), if any, of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region, comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A nucleic acid or protein is “fixed” to a substrate if it is covalently or non-covalently associated with the substrate such that the substrate can be rinsed with a fluid (e.g. standard saline citrate, pH 7.4) without a substantial fraction of the nucleic acid or protein dissociating from the substrate.

As used herein, a “naturally-occurring” nucleic acid molecule refers to an RNA or DNA molecule having a nucleotide sequence that occurs in nature.

Expression of a marker gene in a patient is “significantly” altered from the level of expression of the marker gene in a control subject if the level of expression of the marker gene in a sample from the patient differs from the level in a sample from the control subject by an amount greater than the standard error of the assay employed to assess expression, and preferably at least twice, and more preferably three, four, five or ten times that amount. Expression of a marker gene in a patient is “significantly” higher than the level of expression of the marker gene in a control subject if the level of expression of the marker gene in a sample from the patient is greater than the level in a sample from the control subject by an amount greater than the standard error of the assay employed to assess expression, and preferably at least twice, and more preferably three, four, five or ten times that amount. Alternately, expression of the marker gene in the patient can be considered “significantly” lower than the level of expression in a control subject if the level of expression in a sample from the patient is lower than the level in a sample from the control subject by an amount greater than the standard error of the assay employed to assess expression, and preferably at least twice, and more preferably three, four, five or ten times that amount.

Breast cancer is “inhibited” if at least one symptom of the cancer is alleviated, terminated, slowed, or prevented. As used herein, breast cancer is also “inhibited” if recurrence or metastasis of the cancer is reduced, slowed, delayed, or prevented.

A kit is any manufacture (e.g. a package or container) comprising at least one reagent, e.g. a probe, for specifically detecting a marker gene or peptide of the invention. The manufacture is preferably promoted, distributed, or sold as a unit for performing the methods of the present invention.

Description

The present invention is based, in part, on the identification of proteins which are secreted or otherwise released from breast cancer cells but not from normal (i.e., non-cancerous) epithelial cells. The marker genes of the invention (listed in Table 1) encode such secreted or released proteins. The presence, absence, or level of expression of one or more of these marker genes and/or their gene products in breast cells or associated fluids is correlated with the cancerous state of the tissue. In particular, the level of expression a marker gene in Table 1 is increased in breast cancer cells relative to expression in normal epithelial cells. The invention thus includes compositions, kits, and methods for assessing the cancerous state of breast cells (e.g. cells obtained from a human, cultured human cells, archived or preserved human cells and in vivo cells).

The compositions, kits, and methods of the invention have the following uses, among others:

1) assessing whether a patient is afflicted with breast cancer;

2) assessing the stage of breast cancer in a human patient;

3) assessing the grade of breast cancer in a patient;

4) assessing the benign or malignant nature of breast cancer in a patient;

5) assessing the metastatic potential of breast cancer in a patient;

6) assessing the histological type of neoplasm (e.g. adenocarcinoma) associated with breast cancer in a patient;

7) assessing the indolent or aggressive nature of breast cancer in a patient;

8) making an isolated hybridoma which produces an antibody useful for assessing whether a patient is afflicted with breast cancer;

9) assessing the presence of breast cancer cells;

10) assessing the efficacy of one or more test compounds for inhibiting breast cancer in a patient;

11) assessing the efficacy of a therapy for inhibiting breast cancer in a patient;

12) monitoring the progression of breast cancer in a patient;

13) selecting a composition or therapy for inhibiting breast cancer in a patient;

14) treating a patient afflicted with breast cancer;

15) inhibiting breast cancer in a patient;

16) assessing the breast carcinogenic potential of a test compound; and

17) inhibiting breast cancer in a patient at risk for developing breast cancer.

The invention thus includes a method of assessing whether a patient is afflicted with breast cancer which includes assessing whether the patient has pre-metastasized breast cancer. This method comprises comparing the level of expression of a breast cancer marker gene in a patient sample and the normal level of expression of the marker gene in a control sample, e.g., a sample from a subject having no breast cancer. A significant difference between the level of expression of the marker gene in the patient sample and the normal level is an indication that the patient is afflicted with breast cancer. The breast cancer marker gene is selected from the group consisting of the marker genes listed within Table 1. In particular, the level of expression of the marker genes in Table 1 is increased in breast cancer cells relative to expression in normal breast cells. Although one or more marker genes listed within Table 1 or their encoded proteins may have been described by others, the significance of the level of expression of these marker genes with regard to the cancerous state of breast cells has not previously been recognized.

Any marker gene or combination of marker genes listed within Table 1, as well as any known breast cancer marker genes in combination with the marker genes set forth within Table 1, may be used in the compositions, kits, and methods of the present invention. In general, it is preferable to use marker genes for which the difference between the level of expression of the marker gene in breast cancer cells or breast-associated body fluids and the level of expression of the same marker gene in normal breast cells or breast-associated body fluids is as great as possible. Although this difference can be as small as the limit of detection of the method for assessing expression of the marker gene, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater.

It is recognized that certain markers correspond to proteins which are secreted from breast cells (i.e. one or both of normal and cancerous cells) to the extracellular space surrounding the cells. These markers are preferably used in certain embodiments of the compositions, kits, and methods of the invention, owing to the fact that the protein corresponding to each of these markers can be detected in an breast-associated body fluid sample, which may be more easily collected from a human patient than a tissue biopsy sample. In addition, preferred in vivo techniques for detection of a protein corresponding to a marker of the invention include introducing into a subject a labeled antibody directed against the protein. For example, the antibody can be labeled with a radioactive marker whose presence and location in a subject can be detected by standard imaging techniques.

Although not every marker corresponding to a secreted protein is indicated as such herein, it is a simple matter for the skilled artisan to determine whether any particular marker corresponds to a secreted protein. In order to make this determination, the protein corresponding to a marker is expressed in a test cell (e.g. a cell of a breast cell line), extracellular fluid is collected, and the presence or absence of the protein in the extracellular fluid is assessed (e.g. using a labeled antibody which binds specifically with the protein).

The following is an example of a method which can be used to detect secretion of a protein corresponding to a marker of the invention. About 8×10⁵ 293T cells are incubated at 37° C. in wells containing growth medium (Dulbecco's modified Eagle's medium {DMEM} supplemented with 10% fetal bovine serum) under a 5% (v/v) CO₂, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

It will be appreciated that patient samples containing breast cells may be used in the methods of the present invention. In these embodiments, the level of expression of the marker gene can be assessed by assessing the amount (e.g. absolute amount or concentration) of a marker gene product (e.g., protein and RNA transcript encoded by the marker gene and fragments of the protein and RNA transcript) in a sample of breast-associated body fluid. Examples of breast-associated body fluids include blood fluids (e.g. whole blood, blood serum, blood having platelets removed therefrom, etc.), lymph, ascitic fluid, cystic fluid, urine and nipple aspirates. The breast-associated fluid sample can, of course, be subjected to a variety of well-known post-collection preparative and storage techniques (e.g. fixation, storage, freezing, lysis, homogenization, DNA or RNA extraction, ultrafiltration, concentration, evaporation, centrifugation, etc.) prior to assessing the amount of the marker gene product in the sample.

Preferred in vivo techniques for detection of a protein encoded by marker gene of the invention include introducing into a subject an antibody that specifically binds the protein, or a polypeptide or protein fragment comprising the protein. In certain embodiments, the antibody can be labeled with a radioactive molecule whose presence and location in a subject can be detected by standard imaging techniques.

Expression of a marker gene of the invention may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. Such method may also include physical methods such as liquid and gas chromatography, mass spectroscopy, and nuclear magnetic resonance.

In a preferred embodiment, expression of a marker gene is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein encoded by the marker gene or a polypeptide or a protein fragment comprising the protein, wherein the protein may have undergone none, all or a portion of its normal post-translational modification and/or proteolysis during the course of its secretion or release from breast cells, cancerous or otherwise.

In another preferred embodiment, expression of a marker gene is assessed by preparing mRNA/cDNA (i.e. a transcribed polynucleotide) from cells in a patient sample, and by hybridizing the mRNA/cDNA with a reference polynucleotide which comprises the marker gene sequence or its complement, or a fragment of said sequence or complement. cDNA can, optionally, be amplified using any of a variety of polymerase chain reaction methods prior to hybridization with the reference polynucleotide. Expression of one or more marker genes can likewise be detected using quantitative PCR to assess the level of RNA transcripts encoded by the marker gene(s).

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a RNA transcript encoded by a marker gene of the invention. If polynucleotides complementary to or homologous with a RNA transcript encoded by the marker gene of the invention are differentially detectable on the substrate (e.g. detectable using radioactivity, different chromophores or fluorophores), are fixed to different selected positions, then the levels of expression of a plurality of marker genes can be assessed simultaneously using a single substrate (e.g. a “gene chip” microarray of polynucleotides fixed at selected positions). When a method of assessing marker gene expression is used which involves hybridization of one nucleic acid with another, it is preferred that the hybridization be performed under stringent hybridization conditions.

Because the compositions, kits, and methods of the invention rely on detection of a difference in expression levels of one or more marker genes of the invention, it is preferable that the level of expression of the marker gene is significantly greater than the minimum detection limit of the method used to assess expression in at least one of normal breast cells and cancerous breast cells.

It is understood that by routine screening of additional patient samples for the expression levels of one or more of the marker genes of the invention, it will be realized that certain of the marker genes are over- or underexpressed in cancers of various types, including specific breast cancers, as well as other cancers such as ovarian cancers. For example, it will be confirmed that some of the marker genes of the invention are over-expressed in most (i.e. 50% or more) or substantially all (i.e. 80% or more) of breast cancer. Furthermore, it will be confirmed that certain of the markers of the invention are associated with breast cancer of various stages (i.e. stage 0, I, II, II, and IV breast cancers, as well as subclassifications IIA, IIB, IIIA, and IIIB, using the FIGO Stage Grouping system for primary carcinoma of the breast; (see Breast, In: American Joint Committee on Cancer: AJCC Cancer Staging Manual. Lippincott-Raven Publishers, 5th ed., 1997, pp. 171-180), of various histologic subtypes (e.g. serous, mucinous, endometroid, and clear cell subtypes, as well as subclassifications and alternate classifications adenocarcinoma, papillary adenocarcinoma, papillary cystadenocarcinoma, surface papillary carcinoma, malignant adenofibroma, cystadenofibroma, adenocarcinoma, cystadenocarcinoma, adenoacanthoma, endometrioid stromal sarcoma, mesodermal (Müillerian) mixed tumor, mesonephroid tumor, malignant carcinoma, Brenner tumor, mixed epithelial tumor, and undifferentiated carcinoma, using the WHO/FIGO system for classification of malignant breast tumors; Scully, Atlas of Tumor Pathology, 3d series, Washington D.C.), and various grades (i.e. grade I {well differentiated}, grade II {moderately well differentiated}, and grade III {poorly differentiated from surrounding normal tissue})).

It will thus be appreciated that as a greater number of patient samples are assessed for expression of the marker genes of the invention and the outcomes of the individual patients from whom the samples were obtained are correlated, it will also be confirmed that altered expression of certain of the marker genes of the invention are strongly correlated with malignant cancers and that altered expression of other marker genes of the invention are strongly correlated with benign tumors. The compositions, kits, and methods of the invention are thus useful for characterizing one or more of the stage, grade, histological type, metastatic potential, indolent vs. aggressive phenotype and benign/malignant nature of breast cancer in patients. In addition, these compositions, kits, and methods can be used to detect and differentiate lobular and ductal carcinoma breast cancers.

When the compositions, kits, and methods of the invention are used for characterizing one or more of the stage, grade, histological type, metastatic potential, indolent vs. aggressive phenotype and benign/malignant nature of breast cancer in a patient, it is preferred that the marker gene or panel of marker genes of the invention, whose expression level is assessed, is selected such that a positive result is obtained in at least about 20%, and preferably at least about 40%, 60%, or 80%, and more preferably in substantially all patients afflicted with a breast cancer of the corresponding stage, grade, histological type, metastatic potential, indolent vs. aggressive phenotype or benign/malignant nature. Preferably, the marker gene or panel of marker genes of the invention is selected such that a positive predictive value (PPV) of greater than about 10% is obtained for the general population.

When a plurality of marker genes of the invention are used in the methods of the invention, the level of expression of each marker gene in a patient sample can be compared with the normal level of expression of each of the plurality of marker genes in non-cancerous samples of the same type, either in a single reaction mixture (i.e. using reagents, such as different fluorescent probes, for each marker gene or a mixture of similiarly labeled probes to access expression level of a plurality of marker genes whose probes are fixed to a single substrate at different positions) or in individual reaction mixtures corresponding to one or more of the marker genes. In one embodiment, a significantly enhanced level of expression of more than one of the plurality of marker genes in the sample, relative to the corresponding normal levels, is an indication that the patient is afflicted with breast cancer. When the expression level of a plurality of marker genes is assessed, it is preferred that the expression level of 2, 3, 4, 5, 8, 10, 12, 15, 20, 30, or 40 or more individual marker genes is assessed.

In order to maximize the sensitivity of the compositions, kits, and methods of the invention (i.e. by interference attributable to cells of non-breast origin in a patient sample), it is preferable that the marker gene of the invention whose expression level is examined therein be a marker gene which is tissue specific, e.g., normally not expressed in non-breast tissue.

There are only a small number of marker genes whose expression are known to be associated with breast cancers (e.g. BRCA1 and BRCA2). These marker genes are not, of course, included among the marker genes of the invention, although they may be used together with one or more marker genes of the invention in a panel of marker genes, for example. It is well known that certain types of genes, such as oncogenes, tumor suppressor genes, growth factor-like genes, protease-like genes, and protein kinase-like genes are often involved with development of cancers of various types. Thus, among the marker genes of the invention, use of those which encode proteins which resemble known secreted proteins such as growth factors, proteases and protease inhibitors are preferred.

Known oncogenes and tumor suppressor genes include, for example, abl, abr, akt2, apc, bcl2 α, bcl2β, bcl3, bcr, brca1, brca2, cbl, ccnd1, cdc42, cdk4, crk-II, csflr/fms, dbl, dcc, dpc4/smad4, e-cad, e2f/rbap, egfr/erbb-1, elk1, elk3, eph, erg, ets1, ets2, fer, fgr/src2, flil/ergb2, fos, fps/fes, fra1, fra2, fyn, hck, hek, her2/erbb-2/neu, her3/erbb-3, her4/erbb-4, hras1, hst2, hstf1, igfbp2, ink4a, ink4b, int2/fgf3, jun, junb, jund, kip2, kit, kras2a, kras2b, lck, lyn, mas, max, mcc, mdm2, met, mlh1, mmp10, mos, msh2, msh3, msh6, myb, myba, mybb, myc, mycl1, mycn, nf1, nf2, nme2, nras, p53, pdgfb, phb, pim1, pms1, pms2, ptc, pten, raf1, rap1a, rb1, rel, ret, ros1, ski, src1, tal1, tgfbr2, tgfb3, tgfbr3, thra1, thrb, tiam1, timp3, tjp1, tp53, trk, vav, vhl, vil2, waf1, wnt1, wnt2, wt1, and yes1 (Hesketh, 1997, In: The Oncogene and Tumour Suppressor Gene Facts Book, 2nd Ed., Academic Press; Fishel et al., 1994, Science 266:1403-1405).

Known growth factors include platelet-derived growth factor alpha, platelet-derived growth factor beta (simian sarcoma viral {v-sis) oncogene homolog), thrombopoietin (myeloproliferative leukemia virus oncogene ligand, megakaryocyte growth and development factor), erythropoietin, B cell growth factor, macrophage stimulating factor 1 (hepatocyte growth factor-like protein), hepatocyte growth factor (hepapoietin A), insulin-like growth factor 1 (somatomedia C), hepatoma-derived growth factor, amphiregulin (schwannoma-derived growth factor), bone morphogenetic proteins 1, 2, 3, 3 beta, and 4, bone morphogenetic protein 7 (osteogenic protein 1), bone morphogenetic protein 8 (osteogenic protein 2), connective tissue growth factor, connective tissue activation peptide 3, epidermal growth factor (EGF), teratocarcinoma-derived growth factor 1, endothelin, endothelin 2, endothelin 3, stromal cell-derived factor 1, vascular endothelial growth factor (VEGF), VEGF-B, VEGF-C, placental growth factor (vascular endothelial growth factor-related protein), transforming growth factor alpha, transforming growth factor beta 1 and its precursors, transforming growth factor beta 2 and its precursors, fibroblast growth factor 1 (acidic), fibroblast growth factor 2 (basic), fibroblast growth factor 5 and its precursors, fibroblast growth factor 6 and its precursors, fibroblast growth factor 7 (keratinocyte growth factor), fibroblast growth factor 8 (androgen-induced), fibroblast growth factor 9 (glia-activating factor), pleiotrophin (heparin binding growth factor 8, neurite growth-promoting factor 1), brain-derived neurotrophic factor, and recombinant glial growth factor 2.

Known proteases include interleukin-1 beta convertase and its precursors, Mch6 and its precursors, Mch2 isoform alpha, Mch4, Cpp32 isoform alpha, Lice2 gamma cysteine protease, Ich-1S, Ich-1L, Ich-2 and its precursors, TY protease, matrix metalloproteinase 1 (interstitial collagenase), matrix metalloproteinase 2 (gelatinase A, 72kD gelatinase, 72kD type IV collagenase), matrix metalloproteinase 7 (matrilysin), matrix metalloproteinase 8 (neutrophil collagenase), matrix metalloproteinase 12 (macrophage elastase), matrix metalloproteinase 13 (collagenase 3), metallopeptidase 1, cysteine-rich metalloprotease (disintegrin) and its precursors, subtilisin-like protease Pc8 and its precursors, chymotrypsin, snake venom-like protease, cathepsin 1, cathepsin D (lysosomal aspartyl protease), stromelysin, aminopeptidase N, plasminogen, tissue plasminogen activator, plasminogen activator inhibitor type II, and urokinase-type plasminogen activator.

It is recognized that the compositions, kits, and methods of the invention will be of particular utility to patients having an enhanced risk of developing breast cancer and their medical advisors. Patients recognized as having an enhanced risk of developing breast cancer include, for example, patients having a familial history of breast cancer, patients identified as having a mutant oncogene (i.e. at least one allele), and patients determined through any other established medical criteria to be at risk for cancer or other malignancy.

The level of expression of a marker gene in normal (i.e. non-cancerous) human breast tissue can be assessed in a variety of ways. In one embodiment, this normal level of expression is assessed by assessing the level of expression of the marker gene in a portion of breast cells which appears to be non-cancerous and by comparing this normal level of expression with the level of expression in a portion of the breast cells which is suspected of being cancerous. For example, when mammography or another medical procedure reveals the presence of a lump in the patient's breast, the normal level of expression of a marker gene may be assessed using a non-affected portion of the breast and this normal level of expression may be compared with the level of expression of the same marker gene in an affected portion (i. e. the lump) of the breast. Alternately, and particularly as further information becomes available as a result of routine performance of the methods described herein, population-average values for normal expression of the marker genes of the invention may be used. In other embodiments, the ‘normal’ level of expression of a marker gene may be determined by assessing expression of the marker gene in a patient sample obtained from a non-cancer-afflicted patient, from a patient sample obtained from a patient before the suspected onset of breast cancer in the patient, from archived patient samples, and the like.

The invention includes compositions, kits, and methods for assessing the presence of breast cancer cells in a sample (e.g an archived tissue sample or a sample obtained from a patient). These compositions, kits, and methods are substantially the same as those described above, except that, where necessary, the compositions, kits, and methods are adapted for use with samples other than patient samples. For example, when the sample to be used is a parafinized, archived human tissue sample, it can be necessary to adjust the ratio of compounds in the compositions of the invention, in the kits of the invention, or the methods used to assess levels of marker gene expression in the sample. Such methods are well known in the art and within the skill of the ordinary artisan.

The invention includes a kit for assessing the presence of breast cancer cells (e.g. in a sample such as a patient sample). The kit comprises a plurality of reagents, each of which is capable of binding specifically with a protein or nucleic acid encoded by a marker gene of the invention. Suitable reagents for binding with a protein encoded by a marker gene of the invention include antibodies, antibody derivatives, antibody fragments, and the like. Additional reagents for specifically binding with a protein encoded by a marker gene include any natural ligands of the protein and derivatives of such ligands. Suitable reagents for binding with a nucleic acid encoded by a marker gene (e.g. an hnRNA, a spliced mRNA, a cDNA corresponding to the mRNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for binding an antibody with a protein with which it specifically binds or, for annealing complementary nucleic acids one or more sample compartments, instructional material which describes performance of a method of the invention, a sample of normal breast cells, a sample of breast cancer cells, and the like.

The invention also includes a method of making an isolated hybridoma which produces an antibody useful for assessing whether a patient is afflicted with breast cancer. In this method, a composition comprising a protein encoded by a marker gene or a polypeptide or protein fragment of the protein is used to immunize a vertebrate, preferably a mammal such as a mouse, rat, rabbit, or sheep. The vertebrate may optionally (and preferably) be immunized at least one additional time with the composition, so that the vertebrate exhibits a robust immune response to the protein or parts thereof. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or part thereof. The invention also includes hybridomas made by this method and antibodies made using such hybridomas. An antibody of the invention may also be used as a therapeutic agent for treating cancers, particular breast cancers.

The invention also includes a method of assessing the efficacy of a test compound for inhibiting breast cancer cells. As described above, differences in the level of expression of the marker genes of the invention correlate with the cancerous state of breast cells. Although it is recognized that changes in the levels of expression of certain of the marker genes of the invention likely result from the cancerous state of breast cells, it is likewise recognized that changes in the levels of expression of other of the marker genes of the invention induce, maintain, and promote the cancerous state of those cells. Thus, compounds which inhibit breast cancer in a patient will cause the level of expression of one or more of the marker genes of the invention to change to a level nearer the normal level of expression for that marker gene (i.e. the level of expression for the marker gene in non-cancerous breast cells).

This method thus comprises comparing expression of a marker gene in a first breast cell sample and maintained in the presence of the test compound and expression of the marker gene in a second breast cell sample and maintained in the absence of the test compound. A significantly altered level of expression of a marker gene listed within Table 1 is an indication that the test compound inhibits breast cancer. The breast cell samples may, for example, be aliquots of a single sample of normal breast cells obtained from a patient, pooled samples of normal breast cells obtained from a patient, cells of a normal breast cell line, aliquots of a single sample of breast cancer cells obtained from a patient, pooled samples of breast cancer cells obtained from a patient, cells of a breast cancer cell line, or the like. In one embodiment, the samples are breast cancer cells obtained from a patient and a plurality of compounds known to be effective for inhibiting various breast cancers are tested in order to identify the compound which is likely to best inhibit the breast cancer in the patient.

This method may likewise be used to assess the efficacy of a therapy for inhibiting breast cancer in a patient. In this method, the level of expression of one or more marker genes of the invention in a pair of samples (one subjected to the therapy, the other not subjected to the therapy) is assessed. As with the method of assessing the efficacy of test compounds, if the therapy induces a significant alteration in the level of expression of a marker gene listed within Table 1 then the therapy is efficacious for inhibiting breast cancer. As above, if samples from a selected patient are used in this method, then alternative therapies can be assessed in vitro in order to select a therapy most likely to be efficacious for inhibiting breast cancer in the patient.

As described herein, breast cancer in patients is associated with an altered level of expression of one or more marker genes listed within Table 1. While, as discussed above, some of these changes in expression level result from occurrence of the breast cancer, others of these changes induce, maintain, and promote the cancerous state of breast cancer cells. Thus, breast cancer characterized by an altered level of expression of one or more marker genes listed within Table 1 can be controlled or suppressed by altering expression of those marker genes.

Expression of a marker gene listed within Table 1 can be inhibited in a number of ways generally known in the art. For example, an antisense oligonucleotide can be provided to the breast cancer cells in order to inhibit transcription, translation, or both, of the marker gene(s). Alternately, a polynucleotide encoding an antibody, an antibody derivative, or an antibody fragment, and operably linked with an appropriate promoter/regulator region, can be provided to the cell in order to generate intracellular antibodies which will inhibit the function or activity of the protein encoded by the marker gene(s). Using the methods described herein, a variety of molecules, particularly including molecules sufficiently small that they are able to cross the cell membrane, can be screened in order to identify molecules which inhibit expression of the marker gene(s). The compound so identified can be provided to the patient in order to inhibit expression of the marker gene(s) in the breast cancer cells of the patient.

Expression of a marker gene listed within Table 1 can be enhanced in a number of ways generally known in the art. For example, a gene construct comprising the coding region of the marker gene operably linked with an appropriate promoter/regulator region can be provided to breast cancer cells of the patient in order to induce enhanced expression of the protein (and mRNA) encoded by the marker gene. Expression of the protein can be enhanced by providing the protein (e.g. directly or by way of the bloodstream or another breast-associated fluid) to breast cancer cells in the patient.

As described above, the cancerous state of human breast cells is correlated with changes in the levels of expression of the marker genes of the invention. Thus, compounds which alter expression of one or more of the marker genes listed in within Table 1 can induce breast cell carcinogenesis. The invention thus includes a method for assessing the human breast cell carcinogenic potential of a test compound. This method comprises maintaining separate aliquots of human breast cells in the presence and absence of the test compound. Expression of a marker gene of the invention in each of the aliquots is compared. A significant alteration in the level of expression of a marker gene listed within Table 1 in the aliquot maintained in the presence of the test compound (relative to the aliquot maintained in the absence of the test compound) is an indication that the test compound possesses human breast cell carcinogenic potential. The relative carcinogenic potentials of various test compounds can be assessed by comparing the degree of enhancement or inhibition of the level of expression of the relevant marker genes, by comparing the number of marker genes for which the level of expression is enhanced or inhibited, or by comparing both.

Various aspects of the invention are described in further detail in the following subsections.

I. Isolated Nucleic Acid Molecules

One aspect of the invention pertains to isolated nucleic acid molecules that correspond to a marker gene of the invention. Such nucleic acid molecules comprise sequences of RNA transcripts encoded by the marker gene or portions of such transcripts. Isolated nucleic acids of the invention also include nucleic acid molecules sufficient for use as hybridization probes to identify of RNA transcripts encoded by the marker gene or portions of such transcripts, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The invention also encompasses polynucleotides which differ from that of the polynucleotides described herein, but which produce the same phenotypic effect, such as an allelic variant. These altered, but phenotypically equivalent polynucleotides are referred to as “equivalent nucleic acids.” This invention also encompasses polynucleotides characterized by changes in non-coding regions that do not alter the polypeptide produced therefrom when compared to the polynucleotide herein. This invention further encompasses polynucleotides, which hybridize to the polynucleotides of the subject invention under conditions of moderate or high stringency. Alternatively, the polynucleotides are at least 85%, or at least 90%, or more preferably, greater or equal to 95% identical as determined by a sequence alignment program when run under default parameters.

An “isolated” nucleic acid molecule is one which is separated from other nucleic acid molecules which are present in the natural source of the nucleic acid molecule. Preferably, an “isolated” nucleic acid molecule comprises a protein-coding sequence and is free of sequences which naturally flank the coding sequence in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleotide transcript encoded by a marker gene listed in Table 1, can be isolated using standard molecular biology techniques. Nucleic acid molecule of the present invention also encompass the marker genes of the invention, which can be isolated using standard hybridization and cloning techniques (e.g., as described in Sambrook et al., ed., Molecular Cloning. A Laboratory Manual, 2nd ed., Cold Spring Harbor Laboratory Press, Cold Spring Harbor, N.Y., 1989).

A process for identifying a larger fragment or the full-length coding sequence of a marker gene of the present invention is thus also provided. Any conventional recombinant DNA techniques applicable for isolating polynucleotides may be employed. One such method involves the 5′-RACE-PCR technique, in which the poly-A mRNA that contains the coding sequence of particular interest is first reverse transcribed with a 3′-primer comprising a sequence disclosed herein. The newly synthesized cDNA strand is then tagged with an anchor primer with a known sequence, which preferably contains a convenient cloning restriction site attached at the 5′end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc., Clontech) according to the manufacturer's instructions.

Isolating the complete coding sequence of a gene can also be carried out in a hybridization assay using a suitable probe. The probe preferably comprises at least 10 nucleotides, and more preferably exhibits sequence homology to the polynucleotides of the marker genes of the present invention. Other high throughput screens for cDNAs, such as those involving gene chip technology, can also be employed in obtaining the complete cDNA sequence.

In addition, databases exist that reduce the complexity of ESTs by assembling contiguous EST sequences into tentative genes. For example, TIGR has assembled human ESTs into a database called THC for tentative human consensus sequences. The THC database allows for a more definitive assignment compared to ESTs alone. Software programs exist (TIGR assembler and TIGEM EST assembly machine and contig assembly program (see Huang, X., 1996, Genomes 33:21-23)) that allow for assembling ESTs into contiguous sequences from any organism.

Alternatively, mRNA from a sample preparation is used to construct cDNA library in the ZAP Express vector following the procedure described in Velculescu et al., 1997, Science 270:484. The ZAP Express cDNA synthesis kit (Stratagene) is used accordingly to the manufacturer's protocol. Plates containing 250 to 2000 plaques are hybridized as described in Rupert et aL, 1988, Mol. Cell. Bio. 8:3104 to oligonucleotide probes with the same conditions previously described for standard probes except that the hybridization temperature is reduced to a room temperature. Washes are performed in 6× standard-saline-citrate 0.1% SDS for 30 minutes at room temperature. The probes are labeled with ³²P-ATP trough use of T4 polynucleotide kinase.

A partial cDNA (3′ fragment) can be isolated by 3′ directed PCR reaction. This procedure is a modification of the protocol described in Polyak et al., 1997, Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 5′-B-TCC GGC GCG CCG TTT TCC CAG TCA CGA(30)-3′, contains a poly-T stretch at the 3′ end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the strepavidin-coated magnetic beads. Theoretically, any sufficiently-sized DNA region capable of hybridizing to a PCR primer can be used as well as any other 8 base pair recognizing endonuclease.

cDNA constructed utilizing this or similar modified oligo-dT primer is then processed as described in U.S. Pat. No. 5,695,937 up until adapter ligation where only one adapter is ligated to the cDNA pool. After adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

Gene trapper technology can also be used. The reagents and manufacturer's instructions for this technology are commercially available from Life Technologies, Inc., Gaithsburg, Md. Briefly, a complex population of single-stranded phagemid DNA containing directional cDNA inserts is enriched for the target sequence by hybridization in solution to a biotinylated oligonucleotide probe complementary to the target sequence. The hybrids are captured on streptavidin-coated paramagnetic beads. A magnet retrieves the paramagnetic beads from the solution, leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded DNA. Following transformation and plating, typically 20% to 100% of the colones represent the cDNA clone of interest. To identify the desired cDNA clone, the colones may be screened by colony hybridization using the ³²P-labeled oligonucleotide, or alternatively by DNA sequencing and alignment of all sequences obtained from numerous clones to determine a consensus sequence.

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleotide sequence of a RNA transcript encoded by a marker gene of the invention or a complement of said sequence. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of the nucleotide sequence (RNA or cDNA) of a RNA transcript encoded by a marker gene of the invention or a complement of said sequence. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences of one or more marker genes of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a protein which corresponds to a marker gene of the invention, and thus encode the same protein.

In addition to the nucleotide sequences described in the GenBank and IMAGE Consortium database records described herein, and in Table 1, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide by a marker gene of the invention. Such natural allelic variations can typically result in 0.1-0.5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a RNA transcript of a marker gene of the invention or a portion of said transcript or a cDNA corresponding to said transcript or portion thereof. As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6× sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, 1996 August:1675-1680; Breslauer, et al., Proc. Natl. Acad. Sci. USA, Volume 83, 1986 June: 3746-3750; Van Ness, et al., Nucleic Acids Research, Volume 19, No. 19, 1991 September: 5143-5151; McGraw, et al., BioTechniques, Volume 8, No. 6 1990: 674-678; and Milner, et al., Nature Biotechnology, Volume 15, 1997 June: 537-541, all expressly incorporated by reference.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins encoded by the marker genes of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins encoded by the marker genes of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker gene of the invention or complementary to an mRNA sequence corresponding to a marker gene of the invention. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker gene of the invention to thereby inhibit expression of the marker gene, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a breast-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding by a marker gene of the invention can be designed based upon the nucleotide sequence of a cDNA corresponding to the marker gene. For example, a derivative of a Teirahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural bases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad Sci. USA 93:14670-675.

PNAs can be used in therapeutic and diagnostic applications. For example, PNAs can be used as antisense or anti-gene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the bases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

II. Isolated Proteins and Antibodies

One aspect of the invention pertains to isolated proteins encoded by individual marker genes of the invention, and biologically active portions thereof, as well as polypeptide fragments suitable for use as immunogens to raise antibodies directed against a polypeptide encoded by a marker gene of the invention. In one embodiment, the native polypeptide encoded by a marker gene can be isolated from cells or tissue sources by an appropriate purification scheme using standard protein purification techniques. In another embodiment, polypeptides encoded by a marker gene of the invention are produced by recombinant DNA techniques. Alternative to recombinant expression, a polypeptide encoded by a marker gene of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide encoded by a marker gene of the invention include polypeptides comprising amino acid sequences sufficiently identical to or derived from the amino acid sequence of the protein encoded by the marker gene (e.g., the amino acid sequence listed in the GenBank and IMAGE Consortium database records described herein), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence listed in the NCBI Protein Database records described herein. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, el al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad Sci. USA 85:2444-2448. When using the FASTA algorithm for comparing nucleotide or amino acid sequences, a PAM120 weight residue table can, for example, be used with a κ-tuple value of 2.

The percent identity between two sequences can be determined using techniques similar to those described above, with or without allowing gaps. In calculating percent identity, only exact matches are counted.

The invention also provides chimeric or fusion proteins corresponding to a marker gene of the invention. As used herein, a “chimeric protein” or “fusion protein” comprises all or part (preferably a biologically active part) of a polypeptide encoded by a marker gene of the invention operably linked to a heterologous polypeptide (i.e., a polypeptide other than the polypeptide encoded by the marker gene). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide encoded by a marker gene of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide encoded by a marker gene of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide encoded by a marker gene of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides encoded by individual marker genes of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide encoded by a marker gene of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide encoded by a marker gene of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a protein encoded by a marker gene of the invention. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Antibodies of the invention may be used as therapeutic agents in treating cancers. In a preferred embodiment, completely human antibodies of the invention are used for therapeutic treatment of human cancer patients, particularly those having breast cancer. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide encoded by a marker gene of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide encoded by a marker gene of the invention (e.g., a monoclonal antibody) can be used to isolate the polypeptide by standard techniques, such as affinity chromatography or immunoprecipitation. Moreover, such an antibody can be used to detect the polypeptide (e.g., in a cellular lysate or cell supernatant) in order to evaluate the level and pattern of expression of the marker gene. The antibodies can also be used diagnostically to monitor protein levels in tissues or body fluids (e.g in an ovary-associated body fluid) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified antibodies or fragments thereof, and non-human antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of the present invention, an amino acid sequence encoded by the cDNA of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes a polypeptide of the present invention, the method comprising immunizing a mammal with a polypeptide. The polypeptide used as an immunogen comprises an amino acid sequence selected from the group consisting of the amino acid sequence of the present invention, an amino acid sequence encoded by the cDNA of the nucleic acid molecules of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C.

After immunization, a sample is collected from the mammal that contains an antibody that specifically recognizes the polypeptide. Preferably, the polypeptide is recombinantly produced using a non-human host cell. Optionally, the antibodies can be further purified from the sample using techniques well known to those of skill in the art. The method can further comprise producing a monoclonal antibody- producing cell from the cells of the mammal. Optionally, antibodies are collected from the antibody-producing cell.

III. Recombinant Expression Vectors and Host Cells

Another aspect of the invention pertains to vectors, preferably expression vectors, containing a nucleic acid encoding a polypeptide encoded by a marker gene of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide encoded by a marker gene of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn 10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, p. 119-128, In Gene Expression Technology. Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2NOPC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a “selectable marker” (SM) gene (e.g., for resistance to antibiotics) is generally introduced into the host cells along with the gene of interest. Preferred SM genes include those which confer resistance to drugs, such as G418, hygromycin and methotrexate. Cells stably transfected with the introduced nucleic acid can be identified by drug selection (e.g., cells that have incorporated the SM gene will survive, while the other cells die).

A host cell of the invention, such as a prokaryotic or eukaryotic host cell in culture, can be used to produce a polypeptide encoded by a marker gene of the invention. Accordingly, the invention further provides methods for producing a polypeptide encoded by a marker gene of the invention using the host cells of the invention. In one embodiment, the method comprises culturing the host cell of invention (into which a recombinant expression vector encoding a polypeptide of the invention has been introduced) in a suitable medium such that the polypeptide encoded by the marker gene is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide of a marker gene of the invention have been introduced. Such host cells can then be used to create non-human transgenic animals in which exogenous sequences encoding a marker gene of the invention have been introduced into their genome or homologous recombinant animals in which endogenous gene(s) encoding a polypeptide corresponding to a marker gene of the invention have been altered. Such animals are useful for studying the function and/or activity of the polypeptide corresponding to the marker gene and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid encoding a polypeptide encoded by a marker gene of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a marker gene of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teralocarcinomas and Embryonic Stem Cells. A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g, Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) encoded by or corresponding to a marker gene of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid encoded by a marker gene of the invention. Such methods comprise formulating a pharmaceutically acceptable carrier with an agent which modulates expression or activity of a polypeptide or nucleic acid encoded by a marker gene of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid encoded by a marker gene of the invention and one or more additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker gene or its gene products, or (b) have a modulatory (e.g., stimulatory or inhibitory) effect on the activity of the marker gene or, more specifically, (c) have a modulatory effect on the interactions of a protein encoded by the marker gene (hereinafter “marker protein”) with one or more of its natural substrates (e.g., peptide, protein, hormone, co-factor, or nucleic acid), or (d) have a modulatory effect on the expression of the marker gene. Such assays typically comprise a reaction between the marker gene or the marker protein and one or more assay components. The other components may be either the test compound itself, or a combination of test compound and a natural binding partner of the marker protein.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ′one-bead one-compound′ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad Sci. U.S.A. 90:6909; Erb et al. (1994) Proc. Natl. Acad. Sci. USA 91:11422; Zuckermann et al. (1994). J. Med. Chem. 37:2678; Cho etal. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of the marker protein or biologically active portion thereof. In another embodiment, the invention provides assays for screening candidate or test compounds which bind to a marker protein or biologically active portion thereof. Determining the ability of the test compound to directly bind to a marker protein can be accomplished, for example, by coupling the compound with a radioisotope or enzymatic label such that binding of the compound to the marker protein can be determined by detecting the marker protein compound in a labeled complex. For example, compounds (e.g., substrates of the marker protein) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker protein or a biologically active portion thereof. In all likelihood, the marker protein can, in vivo, interact with one or more molecules, such as but not limited to, peptides, proteins, hormones, cofactors and nucleic acids. For the purposes of this discussion, such cellular and extracellular molecules are referred to herein as “binding partners” or marker protein “substrate”. One necessary embodiment of the invention in order to facilitate such screening is the use of the marker protein to identify its natural in vivo binding partners. There are many ways to accomplish this which are known to one skilled in the art. One example is the use of the marker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al ,1993, Biotechniques 14:920-924; Iwabuchi et al, 1993 Oncogene 8:1693-1696; Brent WO94/10300) in order to identify other proteins which bind to or interact with the marker protein (binding partners) and, therefore, are possibly involved in the natural function of the marker protein. Such marker protein binding partners are also likely to be involved in the propagation of signals by the marker protein or downstream elements of a marker gene-mediated signaling pathway. Alternatively, such marker protein binding partners may also be found to be inhibitors of the marker protein .

The two-hybrid system is based on the modular nature of most transcription factors, which consist of separable DNA-binding and activation domains. Briefly, the assay utilizes two different DNA constructs. In one construct, the gene that encodes a marker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker gene-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colones containing the functional transcription factor can be isolated and used to obtain the cloned gene which encodes the protein which interacts with the marker protein.

In a further embodiment, assays may be devised through the use of the invention for the purpose of identifying compounds which modulate (e.g., affect either positively or negatively) interactions between a marker protein and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is a marker protein identified herein (see Table 1), the known binding partner and/or substrate of same, and the test compound. Test compounds can be supplied from any source.

The basic principle of the assay systems used to identify compounds that interfere with the interaction between a marker protein and its binding partner involves preparing a reaction mixture containing the protein and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the protein and its binding partner. Control reaction mixtures are incubated without the test compound or with a placebo. The formation of any complexes between the protein and its binding partner is then detected. The formation of a complex in the control reaction, but less or no such formation in the reaction mixture containing the test compound, indicates that the compound interferes with the interaction of the marker protein and its binding partner. Conversely, the formation of more complex in the presence of compound than in the control reaction indicates that the compound may enhance interaction of the marker protein and its binding partner.

The assay for compounds that interfere with the interaction of a marker protein with its binding partner may be conducted in a heterogeneous or homogeneous format. Heterogeneous assays involve anchoring either the marker protein or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the marker protein and the binding partners (e.g., by competition) can be identified by conducting the reaction in the presence of the test substance, i.e., by adding the test substance to the reaction mixture prior to or simultaneously with the marker protein and its interactive binding partner. Alternatively, test compounds that disrupt preformed complexes, e.g., compounds with higher binding constants, that displace one of the components from the complex, can be tested by adding the test compound to the reaction mixture after complexes have been formed. The various formats are briefly described below.

In a heterogeneous assay system, either a marker protein or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker protein or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker protein fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker protein or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker protein binding or activity determined using standard techniques.

Other techniques for immobilizing proteins on matrices can also be used in the screening assays of the invention. For example, either a marker protein or its binding partner can be immobilized utilizing conjugation of biotin and streptavidin. Biotinylated marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker protein and its binding partner.

Also within the scope of the present invention are methods for direct detection of interactions between a marker protein and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., test compound) such that its emitted fluorescent energy will be absorbed by a fluorescent label on a second, ‘acceptor’ molecule (e.g., test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker protein and its binding partner can be identified in controlled assays.

In another embodiment, modulators of marker gene expression are identified in a method wherein a cell is contacted with a candidate compound and the expression of mRNA or protein encoded by a marker gene is determined. The level of expression of mRNA or protein in the presence of the candidate compound is compared to the level of expression of mRNA or protein in the absence of the candidate compound. The candidate compound can then be identified as a modulator of marker gene expression based on this comparison. For example, when expression of marker gene mRNA or protein is greater (statistically significantly greater) in the presence of the candidate compound than in its absence, the candidate compound is identified as a stimulator of marker gene expression. Conversely, when expression of marker gene mRNA or protein is less (statistically significantly less) in the presence of the candidate compound than in its absence, the candidate compound is identified as an inhibitor of marker gene expression. The level of marker gene expression in the cells can be determined by methods described herein for detecting marker gene mRNA or protein.

In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a marker gene or marker protein modulating agent, an antisense marker gene nucleic acid molecule, an marker protein specific antibody, or an marker protein binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the breast epithelium). A method for lipidation of antibodies is described by Cruikshank et al. (1997) J. Acquired Immune Deficiency Syndromes and Human Retrovirology 14:193.

The nucleic acid molecules corresponding to a marker gene of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Computer Readable Means and Arrays

The present invention also provides computer readable media comprising the nucleic acid sequence of a marker gene of the invention and the amino acid sequence of a marker protein of the invention (hereinafter collectively “sequence information of the present invention”) . As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon sequence information of the present invention.

As used herein, “recorded” refers to a process for storing information on computer readable medium. Those skilled in the art can readily adopt any of the presently known methods for recording information on computer readable medium to generate manufactures comprising the sequence information of the present invention.

A variety of data processor programs and formats can be used to store the sequence information of the present invention on computer readable medium. For example, the sequence information of the present invention can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the sequence information of the present invention.

By providing the sequence information of the present invention in computer readable form, one can routinely access the sequence information for a variety of purposes. For example, one skilled in the art can use the nucleotide or amino acid sequences of a marker gene of the invention in computer readable form to compare a target sequence or target structural motif with the sequence information stored within the data storage means. Search means are used to identify fragments or regions of the marker gene or protein sequence of the invention which match a particular target sequence or target motif.

The invention also includes an array comprising the nucleotide sequence of a marker gene of the present invention. The array can be used to assay expression of one or more genes, including the marker gene, in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of marker gene expression. Thus, not only tissue specificity, but also the level of expression of a battery of genes in the tissue is ascertainable. Thus, marker genes can be grouped on the basis of their tissue expression per se and level of expression in that tissue. This is useful, for example, in ascertaining the relationship of gene expression between or among tissues. Thus, one tissue can be perturbed and the effect on marker gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more marker genes in the array. This can occur in various biological contexts, as disclosed herein, for example in development and differentiation of breast cancer, tumor progression, progression of other diseases, in vitro processes, such a cellular transformation and senescence, autonomic neural and neurological processes, such as, for example, pain and appetite, and cognitive functions, such as learning or memory.

The array is also useful for ascertaining the effect of the expression of a marker gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more marker genes in normal and abnormal cells. This provides a battery of marker genes that could serve as a molecular target for diagnosis or therapeutic intervention.

VI. Predictive Medicine

The present invention pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the level of expression of polypeptides or nucleic acids encoded by one or more marker genes of the invention, in order to determine whether an individual is at risk of developing breast cancer. Such assays can be used for prognostic or predictive purposes to thereby prophylactically treat an individual prior to the onset of the cancer.

Yet another aspect of the invention pertains to monitoring the influence of agents (e.g., drugs or other compounds administered either to inhibit breast cancer or to treat or prevent any other disorder {i.e. in order to understand any breast carcinogenic effects that such treatment may have}) on the expression or activity of a marker gene of the invention in clinical trials. These and other agents are described in further detail in the following sections.

A. Diagnostic Assays

An exemplary method for detecting the presence or absence of a polypeptide or nucleic acid encoded by a marker gene of the invention in a biological sample involves obtaining a biological sample (e.g. a biopsy of breast tissue or a lump) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide encoded by a marker gene of the invention include enzyme linked immunosorbent assays (ELISAs), Western blots, immunoprecipitations, immunohistochemistry and immunofluorescence. In vitro techniques for detection of genomic DNA include Southern hybridizations. Furthermore, in vivo techniques for detection of a polypeptide encoded by a marker gene of the invention include introducing into a subject a labeled antibody directed against the polypeptide. For example, the antibody can be labeled with a radioactive marker whose presence and location in a subject can be detected by standard imaging techniques.

A general principle of such diagnostic and prognostic assays involves preparing a sample or reaction mixture that may contain a protein or nucleotide encoded by a marker gene, and a probe, under appropriate conditions and for a time sufficient to allow the protein or nucleotide and probe to interact and bind, thus forming a complex that can be removed and/or detected in the reaction mixture. These assays can be conducted in a variety of ways.

For example, one method to conduct such an assay would involve anchoring the protein or nucleotide on the one hand or probe on the other onto a solid phase support, also referred to as a substrate, and detecting complexes comprising the target marker gene or protein and the probe anchored on the solid phase at the end of the reaction. In one embodiment of such a method, a sample from a subject, which is to be assayed for presence and/or concentration of the proteins or nucleotides encoded by the marker genes, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, the protein or nucleotide encoded by the marker gene or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker gene protein or nucleotide or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of complexes comprising the marker protein or nucleotide sequence and the probe anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect complexes comprising a marker protein or nucleotide sequence and the probe without further manipulation or labeling of either component (the marker protein or nucleotide or the probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a protein or nucleotide encoded by a marker gene can be accomplished without labeling either assay component (probe or marker gene) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with the marker protein or nucleotide and the probe as solutes in a liquid phase. In such an assay, complexes comprising the marker protein or nucleotide and the probe are separated from uncomplexed components by any of a number of standard techniques, including but not limited to: differential centrifugation, chromatography, electrophoresis and immunoprecipitation. In differential centrifugation, such complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate such complexes from uncomplexed components. For example, gel filtration chromatography separates molecules based on size, and through the utilization of an appropriate gel filtration resin in a column format, for example, the relatively larger complexes may be separated from the relatively smaller uncomplexed components. Similarly, the different charge properties of such complexes as compared to the uncomplexed components may be exploited to differentiate the complexes from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):14_; Hage, D. S., and Tweed, S. A. J. Chromatogr B Biomed Sci Appl Oct. 10, 1997 ;699(1-2):499-525). Gel electrophoresis may also be employed to separate such complexes from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA encoded by a marker gene can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from breast cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA encoded by a marker gene of the present invention. Other suitable probes for use in the diagnostic assays of the invention are described herein. Hybridization of a mRNA with the probe indicates that the marker gene in question is expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the a marker gene of the present invention.

An alternative method for determining the level of mRNA encoded by a marker gene of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the breast cells prior to detection. In such methods, a cell or tissue sample is prepared/processed using known histological methods. The sample is then immobilized on a support, typically a glass slide, and then contacted with a probe that can hybridize to mRNA encoded by the marker gene.

As an alternative to making determinations based on the absolute expression level of the marker gene, determinations may be based on the normalized expression level of the marker gene. Expression levels are normalized by correcting the absolute expression level of a marker gene by comparing its expression to the expression of a gene that is not a marker gene, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-breast cancer sample, or between samples from different sources.

Alternatively, the expression level can be provided as a relative expression level. To determine a relative expression level of a marker gene, the level of expression of the marker gene is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker gene. The expression level of the marker gene determined for the test sample (absolute level of expression) is then divided by the mean expression value obtained for that marker gene. This provides a relative expression level.

Preferably, the samples used in the baseline determination will be from breast cancer or from non-breast cancer cells of breast tissue. The choice of the cell source is dependent on the use of the relative expression level. Using expression found in normal tissues as a mean expression score aids in validating whether the marker gene assayed is breast specific (versus normal cells). In addition, as more data is accumulated, the mean expression value can be revised, providing improved relative expression values based on accumulated data. Expression data from breast cells provides a means for grading the severity of the breast cancer state.

In another embodiment of the present invention, a polypeptide encoded by a marker gene is detected. A preferred agent for detecting a polypeptide of the invention is an antibody capable of binding to a polypeptide encoded by a marker gene of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from breast cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis, immunohistochemistry and enzyme linked immunoabsorbant assay (ELISA). A skilled artisan can readily adapt known protein/antibody detection methods for use in determining whether breast cells express a marker gene of the present invention.

In one format, antibodies, or antibody fragments, can be used in methods such as Western blots, immunohistochemistry or immunofluorescence techniques to detect the expressed proteins. In such uses, it is generally preferable to immobilize either the antibody, proteins, or cells containing proteins, on a solid support. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from breast cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid encoded by a marker gene of the invention in a biological sample (e.g. a breast-associated body fluid). Such kits can be used to determine if a subject is suffering from or is at increased risk of developing breast cancer. For example, the kit can comprise a labeled compound or agent capable of detecting a polypeptide or an mRNA encoding a polypeptide encoded by a marker gene of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker gene of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide encoded by a marker gene of the invention or (2) a pair of primers useful for amplifying a nucleic acid molecule encoded by a marker gene of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

B. Pharmacogenomics

Agents or modulators which have a stimulatory or inhibitory effect on expression of a marker gene of the invention can be administered to individuals to treat (prophylactically or therapeutically) breast cancer in the patient. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the level of expression of a marker gene of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of expression of a marker gene of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker gene of the invention.

This invention also provides a process for preparing a database comprising at least one of the marker genes set forth in Table 1. For example, the polynucleotide sequences are stored in a digital storage medium such that a data processing system for standardized representation of the genes that identify a breast cancer cell is compiled. The data processing system is useful to analyze gene expression between two cells by first selecting a cell suspected of being of a neoplastic phenotype or genotype and then isolating polynucleotides from the cell. The isolated polynucleotides are sequenced. The sequences from the sample are compared with, the sequence(s) present in the database using homology search techniques. Greater than 90%, more preferably greater than 95% and more preferably, greater than or equal to 97% sequence identity between the test sequence and the polynucleotides of the present invention is a positive indication that the polynucleotide has been isolated from a breast cancer cell as defined above.

In an alternative embodiment, the polynucleotides of this invention are sequenced and the information regarding sequence and in some embodiments, relative expression, is stored in any functionally relevant program, e.g., in Compare Report using the SAGE software (available though Dr. Ken Kinzler at John Hopkins University). The Compare Report provides a tabulation of the polynucleotide sequences and their abundance for the samples normalized to a defined number of polynucleotides per library (say 25,000). This is then imported into MS-ACCESS either directly or via copying the data into an Excel spreadsheet first and then from there into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of a polynucleotide of specifically defined interest.

One skilled in the art may create groups containing one or more project(s) by combining the counts of specific polynucleotides within a group (e.g., GroupNormal=Normal1+Normal2, GroupTumor1+TumorCellLine). Additional characteristic values are also calculated for each tag in the group (e.g., average count, minimum count, maximum count). One skilled in the art may calculate individual tag count ratios between groups, for example the ratio of the average GroupNormal count to the average GroupTumor count for each polynucleotide. A statistical measure of the significance of observed differences in tag counts between groups may be calculated.

C. Monitoring Clinical Trials

Monitoring the influence of agents (e.g., drug compounds) on the level of expression of a marker gene of the invention can be applied not only in basic drug screening, but also in clinical trials. For example, the effectiveness of an agent to affect marker gene expression can be monitored in clinical trials of subjects receiving treatment for breast cancer. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected marker genes of the invention in the pre-administration sample; (iii) obtaining one or more post-administration samples from the subject; (iv) detecting the level of expression of the marker gene(s) in the post-administration samples; (v) comparing the level of expression of the marker gene(s) in the pre-administration sample with the level of expression of the marker gene(s) in the post-administration sample or samples; and (vi) altering the administration of the agent to the subject accordingly. For example, increased administration of the agent can be desirable to increase expression of the marker gene(s) to higher levels than detected, i.e., to increase the effectiveness of the agent. Alternatively, decreased administration of the agent can be desirable to decrease expression of the marker gene(s) to lower levels than detected, i.e., to decrease the effectiveness of the agent.

D. Surrogate Marker Genes

The marker genes of the invention may serve as surrogate marker genes for one or more disorders or disease states or for conditions leading up to disease states, and in particular, breast cancer. As used herein, a “surrogate marker gene” is an objective biochemical marker gene which correlates with the absence or presence of a disease or disorder, or with the progression of a disease or disorder (e.g., with the presence or absence of a tumor). The presence or quantity of such marker genes is independent of the disease. Therefore, these marker genes may serve to indicate whether a particular course of treatment is effective in lessening a disease state or disorder. Surrogate marker genes are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker gene, and an analysis of HIV infection may be made using HIV RNA levels as a surrogate marker gene, well in advance of the undesirable clinical outcomes of myocardial infarction or fully-developed AIDS). Examples of the use of surrogate marker genes in the art include: Koomen et al. (2000) J. Mass. Spectrom. 35: 258-264; and James (1994) AIDS Treatment News Archive 209.

The marker genes of the invention are also useful as pharmacodynamic marker genes. As used herein, a “pharmacodynamic marker gene” is an objective biochemical marker gene whose expression correlates specifically with drug effects. The presence or quantity of expression of a pharmacodynamic marker gene is not related to the disease state or disorder for which the drug is being administered; therefore, the presence or quantity of the marker gene expresson is indicative of the presence or activity of the drug in a subject. For example, expression of a pharmacodynamic marker gene may be indicative of the concentration of the drug in a biological tissue, in that the marker gene is either expressed or transcribed or not expressed or transcribed in that tissue in relationship to the level of the drug. In this fashion, the distribution or uptake of the drug may be monitored by assessing expression of the pharmacodynamic marker gene. Similarly, the presence or quantity of expression of the pharmacodynamic marker gene may be related to the presence or quantity of the metabolic product of a drug, such that the presence or quantity of the marker gene expression is indicative of the relative breakdown rate of the drug in vivo. Pharmacodynamic marker genes are of particular use in increasing the sensitivity of detection of drug effects, particularly when the drug is administered in low doses. Since even a small amount of a drug may be sufficient to activate multiple rounds of marker gene transcription or expression, the amplified marker gene may be in a quantity which is more readily detectable than the drug itself. Also, expression of the marker gene may be more easily detected due to the nature of the marker gene itself, for example, using the methods described herein, antibodies may be employed in an immune-based detection system for a protein encoded by a marker gene, or marker gene-specific radiolabeled probes may be used to detect a mRNA encoded by a marker gene. Furthermore, the use of a pharmacodynamic marker gene may offer mechanism-based prediction of risk due to drug treatment beyond the range of possible direct observations. Examples of the use of pharmacodynamic marker genes in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The marker genes of the invention are also useful as pharmacogenomic marker genes. As used herein, a “pharmacogenomic marker gene” is an objective biochemical marker gene whose expression correlates with a specific clinical drug response or susceptibility in a subject (see, e.g., McLeod et al. (1999) Eur. J. Cancer 35(12): 1650-1652). The presence or quantity of expression of the phannacogenomic marker gene is related to the predicted response of the subject to a specific drug or class of drugs prior to administration of the drug. By assessing the presence or quantity of expression of one or more pharmacogenomic marker genes in a subject, a drug therapy which is most appropriate for the subject, or which is predicted to have a greater degree of success, may be selected. For example, based on the presence or quantity of RNA or protein encoded by a specific tumor marker genes in a subject, a drug or course of treatment may be selected that is optimized for the treatment of the specific tumor likely to be present in the subject. Similarly, the presence or absence of a specific sequence mutation in marker gene DNA may correlate with drug response. The use of pharmacogenomic marker genes therefore permits the application of the most appropriate treatment for each subject without having to administer the therapy.

VII. Experimental Protocol

This section describes the isolation of cDNA clones of marker genes.

Subtracted libraries were generated using a PCR based method that produced cDNAs of mRNAs that are present at a higher level in one mRNA population (the tester) than in a second mRNA population (the driver). Both tester and driver mRNA populations were converted into cDNA by reverse transcription, and then PCR amplified using the SMART PCR kit from Clontech. Tester and driver cDNAs were then hybridized using the PCR-Select cDNA subtraction kit from Clontech. This technique effected both a subtraction and normalization of the cDNA. Normalization approximately equalizes the copy numbers of low-abundance and high-abundance cDNA species. After generation of the subtracted libraries from the subtracted and normalized cDNA, 96 or more cDNA clones from each library were tested to confirm differential expression by reverse Southern hybridization.

Various subtracted libraries were constructed to isolated cDNA clones of different breast cancer marker genes. For isolating cDNA clones of genes expressed at high levels in aggressive or metastatic breast tumors, the subtracted libraries were constructed using tester cDNA generated from breast tumor tissues of patients having poor clinical outcome or aggressive tumors, or from cell lines derived from aggressive breast tumors, and driver cDNA generated from breast tumor tissues of patients having good clinical outcome or indolent tumors, or from cell lines derived from indolent breast tumors. “Poor clinical outcome” is a situation where the patient suffered cancer relapse within five years following breast cancer surgery. “Good clinical outcome” is a situation where the patient remained cancer free for over five years following breast cancer surgery. For isolating cDNA clones of genes expressed at high levels in non-aggressive or indolent breast tumors, the subtracted libraries were constructed using tester cDNA generated from breast tumor tissues of patients having good clinical outcome or indolent tumors, or from cell lines derived from indolent breast tumors, and driver cDNA generated from breast tumor tissues of patients having poor clinical outcome or having aggressive breast tumors, or from cell lines derived from aggressive breast tumors.

In Situ Hybridization Methods

Tissue microarrays (TMAs) were constructed using 4 punches of formalin-fixed and paraffin-embedded tumor samples, arrayed on a total of 5 slides. The TMAs were cut and 4 micron thick sections were put onto glass slides. Probes were constructed for radioactive in situ hybridization (ISH) by designing 26mer oligos (flanked with T7 RNA polymerase sequence for transcription) to the 3′ and 5′ ends of the subtractive library clone insert and generating a template via polymerase chain reaction (PCR). Hybridizations were performed with single-stranded 35S-radiolabeled (5×10⁷ cpm/mL) cRNA probes using the PCR-generated insert as a template. ISH was performed according to the methods in Uncan LM , et.al., Melastatin expression and prognosis in cutaneous malignant melanoma. J. Clin Oncol. (2001) Jan 15;19(2):568-76, which is incorporated herein by reference.

In the poor outcome ISH results, 20 out of 40 poor outcome IDC T1-2N0 tumors tested expressed the marker gene 1041, while in the poor outcome TP results, 6 out of 16 poor outcome IDC T1-2N0 tumors tested expressed the marker gene 1041. In the good outcome ISH results, 9 out of 40 good outcome IDC T1-2N0 tumors tested expressed the marker gene 1041, while in the good outcome TP results, 1 out of 22 good outcome IDC T1-2N0 tumors tested expressed the marker gene 1041. This data suggests that expression of marker gene 1041 is associated with poor clinical outcome.

Summary of the Marker Genes

Table 1 lists 1417 marker genes of the invention. All these marker genes may be used to diagnose breast cancer. Specifically, breast cancers may be diagnosed by examining a patient for over-expression of one or more of these marker genes. The isolation of cDNA clones of these marker genes and certain particular use of these marker genes are further described below.

The cDNA clones of marker genes 1-48 were isolated from subtracted libraries using cDNA from aggressive breast tumor cell lines SKBR-3, HS578T, BT549, MDA321 and MDA435 as the tester, and cDNA from indolent breast tumor cell lines MCF-7, T47D, ZR75 as the driver. These marker genes may be particularly useful in diagnosing aggressive breast tumors. Specifically, aggressive breast tumors may be detected by examining a patient for over-expression of any of these marker genes, more preferably marker genes 31-41, and most preferably marker genes 1-30.

The cDNA clones of marker genes 49-112 were isolated from subtracted libraries using cDNA from indolent breast tumor cell lines MCF-7, T47D, ZR75 as the tester and cDNA from aggressive breast tumor cell lines SKBR-3, HS578T, BT549, MDA321, MDA435 as the driver. These marker genes may be particularly useful in diagnosing indolent breast tumors. Specifically, indolent breast tumors may be detected by examining a patient for over-expression of any of these marker genes, more preferably marker genes 62-101, and most preferably marker genes 49-60.

The cDNA clones of marker genes 113-394 were isolated from subtracted libraries using cDNA from breast tumor tissues of patients having poor clinical outcome as the tester and cDNA from tumor tissues of patients having good clinical outcome as the driver. Accordingly, these marker genes may be particularly useful in diagnosing metastatic or aggressive breast tumors or to predict cancer relapse following breast cancer surgery. Specifically, breast cancer metastasis or aggressive breast tumors can be detected, or increased chance of cancer relapse following breast cancer surgery can be predicted, by examining a patient for over-expression of any of these marker genes, preferably marker genes 132-365, more preferably marker genes 126-131 and most preferably marker genes 113-125.

The cDNA clones of marker genes 395-506 were isolated from subtracted libraries using cDNA from breast tumor tissues of patients having good clinical outcome as the tester and cDNA from breast tumor tissues of patients having poor clinical outcome as the driver source. Accordingly, these marker genes may be used to diagnose indolent tumors or to predict efficacy or success of breast cancer surgery. Specifically, indolent breast tumors can be detected or the success of breast cancer surgery can be predicted, by examining a patient for over-expression of any of these marker genes, more preferably marker genes 476-497 and most preferably marker genes 395-475.

The cDNA clones of marker genes 507-611 were isolated from subtracted libraries using cDNA from breast tumor lymph node metastatic tissues as the tester source and cDNA from indolent (colloid and tubular) breast tumor tissues as the driver source. Accordingly, these marker genes can be used to diagnose breast cancer metastasis or aggressive breast tumors. Specifically, breast cancer metastasis or aggressive breast tumors can be detected by examining a patient for over-expression of any of these marker genes, more preferably marker genes 550-603 and most preferably marker genes 507-603.

The cDNA clones of marker genes 612-767 were isolated from subtracted libraries using cDNA from indolent (colloid and tubular) breast tumor samples as the tester source and cDNA from breast tumor lymph node metastatic tissues as the driver source. Accordingly, these marker genes can be used to diagnose indolent breast tumors. Specifically, indolent breast tumors can be detected by examining a patient for over-expression of any of these marker genes, more preferably marker genes 710-762 and most preferably marker genes 612-709.

The cDNA clones of marker genes 768-1055 were isolated from subtracted libraries using cDNA from T1N1 breast tumor tissues (i.e., tissues of breast tumors 2.0 cm or less in greatest dimension with regional lymph node metastasis) as the tester source and cDNA from T1N0 breast tumor tissues (i.e., tissues of breast tumors 2.0 cm or less in greatest dimension with no regional lymph node metastasis) of patients having good clinical outcome as the driver source. Accordingly, these marker genes can be used to diagnose aggressive or metastatic breast tumors. Specifically, aggressive or metastatic breast tumors can be detected by examining a patient for over-expression of any of these marker genes, preferably marker genes 839-1029, more preferably marker genes 826-838, and most preferably marker genes 768-825.

The cDNA clones of marker genes 1056-1417 were isolated from subtracted libraries using cDNA from breast tumor tissues of patients having good clinical outcome as the tester source and cDNA from T1N1 breast tumor tissues as the drive source. Accordingly, these marker genes can be used to diagnose indolent breast tumors or predict efficacy of breast cancer surgery. Specifically, indolent breast tumors can be detected or the success of breast cancer surgery can be predicted by examining a patient for over-expression of any of these marker genes, preferably marker genes 1180-1387, more preferably marker genes 1174-1179 and most preferably marker genes 1056-1173.

Other Embodiments

Those skilled in the art will recognize, or be able to ascertain using no more than routine experimentation, many equivalents to the specific embodiments of the invention described herein. Such equivalents are intended to be encompassed by the following claims.

All publications including journal references, patents and databases are expressly incorporated by reference.

TABLE 1

| | |
|---|---|
| Sequence 1 | cMhvSF008a12 |

ACTATAGGGCGAATTGGAGNTNCCCGCGGTGGCGGCCGAGGTACCGGAGACAGGTGCAGTCCCTC
ACCTGTGAAGTGGATGCCCTTAAAGGAACCAATGAGTCCCTGGAACGCCAGATGCGTGAAATGGA
AGAGAACTTTGCCGTTGAAGCTGCTAACTACCAAGACACTATTGGCCGCCTGCAGGATGAGATTCA
GAATATGAAGGAGGAAATGGCTCGTCACCTTCGTGAATACCAAGACCTGCTCAATGTTAAGATGG
CCCTTGACATTGAGATTGCCACCTACAGGAAGCTGCTGGAAGGCGAGGAGAGCAGGATTTCTCTG
CCTCTTCCAAACTTTTCCTCCCTGAACCTGAGGGGAAACTAATCTGGATTCACTCCCTCTGGTTGAT
ACCCACTCAAAAAGGACACTTNTGATTAAGACGGTTGAAACTAGAGATGGACAGGTTATCAACNG
AAACTTNTCAGCATCACGATGACCTTGAATAAAAAATTGCACACACTCAGTGCAGCAATATATTAC
CAGCAAGGAATAAAAAGAAATCCATATCTTAAAGAAACAGCTTTCAAGTGCCTTTCTGCAGTTTTT
TCAGGAGCCGCAAGATAGATTTTGGAATAGGAAATAAGCTCTAGTTTNTTAACAACCCGACACTTC
TACAAGATTTANNAAAAAAGTTTACCAACAATAATCTAAGTTTACAGAAAAAATCTTGNGCTATA
AATACTTTTTAAAAAGGGATTTTGAATANCCATTAAAAACTGCCTTTTTTTTTTCCAGCAANGTNTT
CAACCAACTTTGGGTTCTGGTTTAATAAAATTTTTGGAAAAAA

| | |
|---|---|
| Sequence 2 | cMhvSF008c12 |

NGGCGAATTGGAGCTCCCCGCGGNGGCGGNCGAGGTACACAGTCAGTGTGGNTGNCTTGCACGAT
GATATGGAGAGCCAGCCCCTGATTGGAACCCAGTCCACAGCTATTCCTGCACCAACTGACCTGAA
GTTCACTCAGGTCACACCCACAAGCCTGAGCGCCCAGTGGCACCACCCAATGTTCAGCTCACTGGA
TATCGAGTGCGGGTGACCCCCAAGGAGAAGACCGGACCAATGAAAGAAATCAACCTTGCTCCTGA
CAGCTCATCCGTGGTTGTATCAGGACTTATGGTGGCCACCAAATATGAAGTGAGTGTCTATGCTCT
TAAGGACACTTTGACAAAGCAGACCAGCTNAAGGGAGTTGTCACCACTCTTGGAGAATGTCAGCC
CACCAAGAAAGGGCTCGTGTGACAAGATGCTTACTGGAGACCACCATCACCATTAGCTGGAGAAC
CAAGACTGAGACGATCACTGGCTTCCAAAGTTGATGCCGTTCCAANCCAATGGGCCNAGACTTCA
ATTCNANANAAACCATTAAGCCAGATGTCAGAAGCTTCCCCATTACANGTTTACAACCAGGCCCTT
GCTACAAAGAATCTACCCTGTCCCNNGGGCCGNTNTAGNAACTAGGGGGATNCCCCCNGGCCTGG
GAGGGAATTTNGATTTTNANCCTTNTTCGATTACCCGNCNANCCNTNTAGGGGGGGGGNCCCGGA
NCCCCACCTTTTNTTNCCTTTNTTGNNGGGNTNAATTTGGGGGGNTTNGGGNAAATAATGGGAATA
AANTNNTTCCNTGGNGNAAATTGNNTTCCCCTCCNATTNCNAAAAAANAAAAACCGGGGNAANAA
AAAGTANNNNGGGGGGGGCCNNANNGGCCCCCCCCCCCCCCCCCCC

| | |
|---|---|
| Sequence 3 | cMhvSF008g12 |

CCCCGCGGTGGCGGCCCGAGGTACAACAAAGCAATGTTACCTTACCATAGGCCTTAATTCAAACTT
TGATCCATTTCACTCCAATGACGGGAGTCAATGCTACCTGGGACACTTGTATTTGTAAATTCTGATT
TAGCTTATTGTAGACTTGTGCCTACTTTGTCATGAGGGTTTGACTTCTGCATTCTTCGTGGCTTTCCT
TCCTTTGGCTTAGGTTTGCTAAAGCTAGAAGATTCAATTGCTCTTTACAGACTTATGAGGAAGATA
GACTTTGTAACGCAGATGTCACTTCTCATGCCACCCTGCCCTGGTTAGCTCTTCTGGAGGAATACTG
CAGATAAGAAAAATAGTTATTTGGGAGGCTCCCTCAAGTGTGGTAGGAATTGAGACTAACACAAT
TTTGGTTAAAGTCCACTGAGGTATGAGTTTATAGAACTCCACTGTATGTATCCAGCTATACTAAAA
CATTTTGCCAAGACACTGGAGGACTCTTTCATTATCTACTGGGAAAGAATAAGACTTAGAGGCTTT
TTAATAAGTTNCTGGGATTGGGTGGGGTAAAAATCATGGAGTTAAAAAAAGACTTGGGGGGAGAA
AGGAAAACCTGTTAAANGTTACATTTAATTTTGGAATTTCNCCNCNNTTGTCAACCTTACTTACAG
GNTNCAATGGCCAAATAAAAAGTTANAAAAAGTTTGGNAGAAATGCTTTCNANGTTTTTNAAAAG
AACCAANGGACCNTNNGCCCCCTTTNNAAAAAAANANNGAACCCCCNCNCGGCCGGGNANNTNT
NTTTANNCTTTTTTTTCCCCCCCCCCCCCTGGGGGGGGGGCNCGGCNCCCCTTTTNTTCCCTTTTTG
GGGGGG

| | |
|---|---|
| Sequence 4 | cMhvSF010e04 |

CCGCGGTGGCGGCCGAGGTACTCCAGGCCGGGACTCAGGTTATCAAAAGTGCAGGAGCTCTGATC
AGCATGGACCACTTCTTCCAAAGAATTTCCCTGCTGGCCGTTTGTAGGGGTTGTGGTAATTCTATA
ACCAGTAATGTCTGGGGTGGTGCTCCTCTCCCAGGAGACTGTGAGCACTCCAGTGTCAGGGTTTGC
CTCCAGATGCAAGTTTGTTGGTGGAGACAATGGTGTCACCACTTTGTTTACAATTGGCGCATCTCTT
TCCTGTCCATCTCTCAGGACTTGGATGGTGTAGACGTATTCTACTCCTGGAGTCAAGCCGGACACA
ACGATGCTTTCTGAGTCTGAAAGTCACNTTTTCGNGGNGCCTTTCCTTCCCTGGCNTTGGNCCGAA
CCCTCGGNCCGNTTTTANAACTTAGTGGAATCCCCCGGGCTTGCAAGGAAATTCAATATCAAACCT
TATCCGATACCCGTCAACCTCNAGGGGGGGGGCCCGGTACCCAACCTTTTGTTCCCTTTAAN

| | |
|---|---|
| Sequence 5 | cMhvSF010f04 |

TTAGGGCGAATTGGAGCTCCCCGTTGTGGCGGCCGAGGTACTGTGGATATTTAAAATATCACAGTA
ACAAGATCATGCTTGTTCCTACAGTATTGCGGGCCAGACACTTAAGTGAAAGCAGAAGTGTTTGGG
TGACTTTCCTACTTAAAATTTTGGTCATATCATTTCAAAACATTTGCATCTTGGTTGGCTGCATATG
CTTTCCTATTGATCCCAAACCAAATCTTAGAATCACTTCATTTAAAATACTGAGCGGTATTGAATAC
TTCGAAGCAGAACAGGCAATGTGCAGCCCTCATTTATGAGAAAACCCTCAGGAAACTCCCAGGGT
GATGCTTGGAGAAGCTGTGAGTTGAGCTGAAGCTGGAGAACTTTCCTCCAGANCCAAANGGCTTT
AAGAAAGGAAAGGAAGAACTCTTAACCTGGGTTCTGCTTAACATCACTCCAAGTTTAANAATGGG
ATCTTGGCCAGAAAAGACCATGCCTTTGTTCCTCTGGAATTGGNAAAAGAATGATTTACTCTCCGG
GAATCTTCTCTGTCAACCTGTACCTNNCCCGCTCTAAAACTAGTTGGATCCCCCGGNCTTCNAGGA
ATTCCATATCAAACCTTATCNATACCCNNCNACCTCNANGNGGGNCCNGNTACCCANCTTTTNTT

| | |
|---|---|
| Sequence 6 | cMhvSF011c10 |

AATACGACTCACTATAGGGGCGAATTGGAGCTCCCCGCGGTGGCGGCCGAGGTACCCAGAAGTGT
CCTGGAATGGGGCCCATGAGATGGTTGTCTGAGAGAGAGCTTCTTGTCCTACATTCGGCGGGTATG
GTCTTGGCCTATGCCTTATGGGGGTGGCCCGTTGTGGGCGGTGTGGTCCGCCTAAAACCATGTTCC
TCAAAGATCATTTGTTGCCAACACTGGGTTGCTGACCAGAAGTGCCAGGAAGCTTAATACCATTTC
CAGTGTCATACCCAGGGTGGGTGACGAAAGGGGTCTTTTGAACTGTGGAAGGAACATCAAGATCT
CTGGTCCATGAAAATTGGGGTGTGGAAGGGTTACCAATTGGGGAAAGCTCGTCTGTCTTTTTCCTT
CCAATCAAGGGCTCCTCTTCTGATTATTCTTCAGGGCAATGACATAAATTGTATATTCGGTTCCCGG
TTCCAGGCCAGTAATAATAGCCTNTGTGACACCAANGGCGGGGCCCA

| | |
|---|---|
| Sequence 7 | cMhvSF013d01 |

CCTGCCGACGTACTTNTGAACAATTATCTCCTCCTGATCACTATTTCNTACTTNGCTTTAAAAANCC
AAAGTTCACAAAGAGAGGGGGAGNANNNGGGGGACTTTTATTCCAATANAAAANATGGANTAAG
TTNTANGGNAGAANNTTGTTCAGTNCGGATNNAAATCTCTATGAAAAGTAAATTCCTTGATNACTG
GTATGACTATAANTCTCTGTTATCNGATACGAGGNANAAACTGCAAGCTGACTAGCATGTTCTGAG
AATCAGCCATTCCTAAAAATTTTATAAACACNNGATACTNTANACNGGANAATGGGACCGCNCCC
AATAAACANATATTTGNGAAAAATGCATCCACA

| | |
|---|---|
| Sequence 8 | cMhvSF017c09 |

ACTCCTATAGGGCGAATTGGAGCTCCCCGCGGTGGCGGCCCGAGGTACTCATCCCTACTGTTATAG
CTGGAGAGGATTTGGGTATTGAAGCAGGGAGGGGCAGATCCCACGAATNGACTGCAGATCTGGAA
TAATAAGTAAGGGGGTAGATCTGCCCATANAGCTCACTTTAACCGGCCTATACTCCTACAAGGAAT
TGGGGTAGGGATCTTCTACTCAGCCTTGCCACAATAGAATGGCCAATGCCCTTCTAGTATGTTTGG
TGAAGGTCTTGAAGGCCCATTTCCCCCATCCACCCTGGGGGAGAAATTGAGTCCCTAAAGTCAACG
ACAAGGCTTATTGAGGCTGAGTTTGCAACAGATCCCGATCTGGGAGGTAGAAACAAAAATGACTG
AACATCTTTTTATCCCCCAATCGTTACAAAGCCTAAATAACTCTAAACGGGATGGGAGGGCAAATT
TTANGTCAAGTTGACATCCTGGAGAAAATATCCTAGGTCCTGTCTCATTCCCTAGACCGCATAACA
CTCCAACCCGTGTAAATCTCAAGGACCCTTGAAAAAGACAGTGGGTAGGGGAAGAAGGAAGGGG
AGCTAGCTTTCCAACCTACTCCACACTTGACTTCCCATANGACAACCAGTAAGTGTAANGGGCATT
TGCAAAATCAAGTGGAAAGTCCTTGGNCGCT

| | |
|---|---|
| Sequence 9 | cMhvSF021f05 |

CGAGGTACCGGAGACAGGTGCAGTCCCTCACCTGTGAAGTGGATGCCCTTAAAGGAACCAATGAG
TCCCTGGAACGCCAGATGCGTGAAATGGAAGAGAACTTTGCCGTTGAAGCTGCTAACTACCAAGA
CACTTTGGCCGCCTGCAGGATGAGATTCAGAATATGAAGGAGGAAATGGCTCGTCACCTTCGTGA
ATACCAAGACCTGCTCAATGTTAAGATGGCCCTTGACATTGAGATTGCCACCTACAGGAAGCTGCT
GGAAGGCGAGGAGAGCAGGTAGGGAACTCAGACTTGGATGCGTGAACTAATGGTGACCATTTGTT
AGGCCCTGTGCCACTGGGCTCTAAGCAGTGTCACATTTAATCTTTAGAAAGTTTCTTTGAGGTAAC
TGCTTTCCACTTTTTGTAGAGGAGGAATTTGAATTGAGAGAGAGTAAGTGACTTGCTGAAAAAGGG
TTAATCAACAGCAGAGCTGGGATTTGAACCCATAACTCTGTCAAAGCCTNCACTCCTAACTCCTGT
TCATGCTCTGTGGAGAAAATGCTTGTAGTACATATTTTAAATGTACCTT

| | |
|---|---|
| Sequence 10 | cMhvSF027h12 |

GNTCNCNNNTGNCGNAANTNTATATAGCNCTNATCTNTNCGGNANCACNTNCANGGGGGNCCCCN
GCACCNACTNTTCNTACCCTTNATNNAGGGTTANTNGCACGCTTGNCCNNNNNATGGACANACTN
TANTTNNTGAGCTCACTGGATATCGAGTGCGGGTGACCCCCAAGGANAANACCGGACCAATGAAA
GAAATCAACCTTGCTCCTGACAGCTCATCCGTGGTTGTATCAGGACTTATGGTGGCCACCAAATAT
AAANTGAGTGTCTATGCTCTTAAGGACAC

| | |
|---|---|
| Sequence 11 | cMhvSF031g09 |

GGAGCTCCCCCGCGGTGGCGGCCGAGGTACTCAGAAGTGTCCTGGAATGGGGCCCATGAGATGGT
TGTCTGAGAGAGAGCTTCTTGTCCTACATTCGGCGGGTATGGTCTTGGCCTATGCCTTATGGGGGT
GGCCGNTGTGGGCGGGTGGTCCGCCTAAAACCATGTTCCTCAAAGATCATTTGTTGCCCAACACTG
GGTTGCTTGACCAGAAGTGCCAGGAAGCTGAATACCATTTNCAGNGTCATACCCAGNGTGGGTGA
CGAAAGGGGTCNTTTGAACTGTGGAAAGGAACATCCAAGATCTCTGGTCCATGAAGATTGGGGTG
TGGAANGGTTACCAGNTGGGGAAGCTCGTCTGTCTTTTTCCTTCCA

| | |
|---|---|
| Sequence 12 | cMhvSF031g12 |

CGACTCACTATAGGGGCGAATTGGGAGCTCCCCCGCGGTGGCGGCCCGAGGTACCTGTTCGCATTG
CAGAATATAAAACTTGGTTTACACTCTATAAAAAATAACCAATATCCAAATTCAAGAGAGCTAGC
ATTCACAGAACACACAATATGGGTGTGTANCTACTGTTCACCAGCCTCAGGCTNGATTTAAACAAA
CAAACAAAAAAAAAATTTNAAAGGGATCATTCAAGATGACCGTATAATGCTTGCTGCTGTCTTTGC
AAATTAAGGTTTGCTTTTCAAGTGCATGATTTTAACATAAGGCCTGGGCTCTCTGCACCTAGTGAG
GTGTGAGGCTCTNTTGCCCACAGTNCACACTNTNACTTAACTAAGCCAGAGTTGGGNGGCATTATT
AAATTATCACTGGTNTTCTTAATAGTNAAAATGGGGGAACCCAGANGGCAGGAAATTTNCATTCC
CTATATTTGGGGCTAAACCTAAAAGAGTATATCCCTTTCAAAGAGCTTAAGTGCCT

| | |
|---|---|
| Sequence 13 | cMhvSF031g12 |

TGANGGAATTCGATATCAAAGCTTATCGGTTNCCGGCCACCTCNAGGGGGGG

| | |
|---|---|
| Sequence 14 | cMhvSF033g12 |

CGCGGTGGCGGCCGAGGTACCGGAGACAGGTGCAGTCCCTCACCTGNGAAGTGGNTGCCCTTNNA
GGNACNACTGAGTGCCTGNATNNCCNGNNTCCACCAAGAGGTGCNACCTNCAACATCATANTGCT
GGTAACTACCAAGACACTATTGGCCNGCCTGCAGGANGAGATTCANAATATGAAGGAGGAAATGG
CNCGTAANNTTTGAGNATACCNANACCTGNTTAANGGTTANANNNCCCTTGACATTGNCAATGCC
ACCTACGGGAANCTGTNGGAANGNNAGGANAGCNAGANTTTTNTGCCTNTTNCAAACTTTTCTCC
CTTGAACCTGAGGGGAAACTAATCTGGATTCACTTCCCTCNGGTTGATACCCACTCAAAAAGGACA
CTTTTGATTAAGACGGNTGAAACTAGAAGATGGACAGGGTTATCAACGAAACTTCTCAACATCAC
CGATGACCTTGAATAAAAATTGCGCACCCTCAGTGCANGCAATATATTTCCAGCAAGAATAAAAA
AGAAATTCCATATCNTAAAGAAACAGCTTTCAATGCCTTTCTGCAGTTTTTTCANGGAGCCGCAAG
ATTNATTTTGGGAATAGGGAATTNAAGCTTTTAGTTTCTTAACAAACCGACACTTCTNACCAAGAT
TTAATAAAAAAAGTTTCAACCTTAATCTTAGTTTAACAGAAAAAATCTTGGNGCTTANAATACTTT
TTAAAAAGGNATTTTTGGAATCTTATTAAAAACTGGTTTTTTTTTT

| | |
|---|---|
| Sequence 15 | cMhvSF053c06 |

CCGCGGTGGCGGCCGAGGTACGATATACGAAGACTCTGAGCTGTTTGCCTCCGATGGGTTTCCAAG
TATTTTGCCCGTTGTAAGCTCATTAAGGGCCAACTTTTACTTTCAATATGTGATTCTGCAGAATTAA
TTTAAGGAGGCGCTGATCATGCTGAGAGTATCAATCAGAAAAATGCATTTATTCACAGGTGCCAGC
AAAGTGTATTCTCCATCTGGCCTCAAAACAGATGCCCAGCCTAATTGGGCCACAAAGATCCCGTGA
AGGTGGTTTTGCTGGTTTNCAAGCCAGCTCAATAACTTGGTTTGGCAGAATCAAGGAATTAAGGAC
CTGATCAATCAAATGGGATCACACCATTATTTGTCACAATATCCCTTTTTGGTCACCATTTTGAATT
CCATTAACTGGTATACTGTCACCGTCACATNCTATCTCAATTGNAT

| | |
|---|---|
| Sequence 16 | cMhvSF053d08 |

ATTGGAGCTCNCCGCGGTGGCGGCCNAGGTCCTGTTCGATTGCAGAATATAAACTTGGTTTACCTC
TATAAAAATACCATATCAAATTCAAGAGAGCTAGCATCCAGAACACCAATATGGGTGTGTAGCTC
TGTCACCACCTAGNTTGATTTAAACAAACAAACAAAAAAAAAATTTCAAAGGGATCATTCAAAGA
TGACCCGTATAATGCTTGCTGCTGCTTTGCAGATTAAGGGTTGCTTTTCAAAGTGCATGATTTTAAC
ATAAGGCCTGGGCTCTCTGCCCTAGTGAGGTGTGAGGCTCTCTTGCCACACAGTTCACACTCTACT
TAACTAAGCCAGAGTTGGTGGCATTATTAAATTATCACTGGTCTTCTTAATAGTAAAAAATGGGGA
ACCCAGANGGCAGGAAATTTCCATTACCCTATATTGGGGCTAAACTTAAAAAGAGTATATCCACTA
TCAAGAGCTTAGTCCTCGGCCGCTCTAGAACTAAGTGGATCCCCCG

| | |
|---|---|
| Sequence 17 | cMhvSF062b03 |

TNCTATAGGGCGAATTGGAGCTCCCCGCGGTGGCGGCCGAGGTACTGTGGATATTTAAAATATCAC
AGTAACAAGATCATGCTTGTTCCTACAGTATTGCGGGCCAGACACTTAAGTGAAAGCAGAAGTGTT
TGGGTGACTTTCCTACTTAAAATTTTGGTCATATCATTTCAAAACATTTGCATCTTGGTTGGCTGCA
TATGCTTTTCCTATTGATCCCAAACCAAATCTTAGAATCACTTCATTTAAAATACTGAGCGGTATTG
AATACTTCGAAGCAGAACAGGCAATTTGCATCTTGGTTGGCTGCATATGCTTTCCTATTGATCCCA
AACCAAATCTTAGAATCACTTCATTTAAAATACTGAGCGGTATTGAATACTTCGAAAGCAGAACAG
GCAAATGTGCAGCCCTCATTTATGAAGAAAACCCTTAGGGAAACTTCCAGGGGTGATG

| | |
|---|---|
| Sequence 18 | cMhvSF063h08 |

TCCCCGCGGTGGCGGCCGAGGTACAGTCCTGATTGCATCATAATTGTGGTTTCCAACCCAGTGGAC
ATTCTTACGTATGTTACCTGNAAACTAANTGGATTACCCAAACACCGCGTGATTGGAAGNGGATGT
AATNTGGATTNTGCTCTATANCACNACCTTATGCGCTGAGAAACTTGANCATNNATCCCNCCNTGG
TNACATGGATGNANTATGGCTNAACCCAACCTANNGATNACTCNTGCTTTGACCCCTACACGAATG
TCTGAATCAGGCTTTAAACTGTTGTGCCAGTGCTTAGGCTTTG

| | |
|---|---|
| Sequence 19 | cMhvSF073c02 |

GGGCGAATTGGAGCTCCCCGCGGTGGCGGCCGAGGTACACAGTCAATGTGGTTGCCTTGCACGAT
GATATGGAGAGCCAGCCCCTGATTGGAACCCAGTCCACAGCTATTCCTGCNCCAACTGACCTGAA
GTT

| | |
|---|---|
| Sequence 20 | cMhvSF087d03 |

TTAGGGCGAATTGGAGCTCACCGCGGTGGCGGCCGAGGTACGTCACGCAGGGCAGCACGTGAGGT
CAAGGCTTGGAAACATCCACATAGATTTGGACATGCTGTTCCTGAATNTGAGCCTGCANCTCCTGG
ATTTCCTCTNCGTGGAGTTTCTTCAAAAAGGCAATCTNTTCTTGCAAAGATTCCACTTTGNGTTNAA
AGGCCAAGAACNTGCCAAAAGACCNAATTTGTNAACAATCCTGNNCTTGAAAAGAATTGNANGGT
GGTTTTCGGNTTNCTCTNTNTGAAGCATNTGNCTNCTGCAATTNCTCCCGGAGGCGCATGATGACC
TNNGNCAGGNNGNNNNGCTCNANCTCNNCNCGGGCTNTGNCGANTGGTTAGNTGGTCCACCTGCC
CGGGCGGNCGCTNGACTCTAGAACTAG

| | |
|---|---|
| Sequence 21 | cMhvSF092d08 |

CCGCGGTGGCGGCCGAGGTACANNAACTGNTTGNATANCTAGNNTNTCATNNTGNGAGGTAATAN
CANCAAANCTAANTCNNNNAAANANCTNATGTGCATTANNANTNGGTNGAATGTCANNNNAATN
NNNNNNAGTNTNGNANNNANNTNACNATCAANNTACAAAGTGNCTTGANGCCNGNNNGGCCNNN
TGCACANTGNANTGACAATNCNNGCNNCTGNNCTGANNTTNTTNANGANTCNNCTGGNATNGATN
CNCNATNNNANNTNNNTTNCCTGGCCACCACACNCAATACCTTGCTGGNATNATGGNAGNCNNCA
CGTGCCAGGATTACCGGCTACATCATNAAGTATGAGAAGCCTGGGTNTCCTCCCANAGAAGTGGT
CCCTNGGCCCCGCCCTGGTGTNACAGAGGCTACTATTACTGGCCTGGAACCGGGNAACCGAATAT
ACAATTTATGTNATTGTCCTGAANAATAATCAGAAAGAGCNAGCCCCTNATTGGAAGGAA

| | |
|---|---|
| Sequence 22 | cMhvSF100f07 |

GCGGNGGCGGCCGAGGNCCATTTNTACGGGGAGACAAAACCCNAANCCCGNGANACCCANGCAA
NNACGACGAANCGCTGNTTACNGNNAACGGGAAGNAACCGCCCNCNANAAAAAAGACAAAGAAC
CAGGCGCATANACNANANANGGGGNGGGNCCAANGCCCATNTGTNCAGGGCCCTTTTTCNGAAA
ACNGGGCACCACAANGAAAAACCCCAGCACNNGGNAGAACNGGNACAAAAAGACCAGCNGNGG
ACAGAAAACGACGGCGNCAAAAGNAAGNNGCCCAGGGNANANGANAANGGAAGGAAGGAANGG
CCGCCCAGNANNAGGGCCCAAGGNCCAAGAGGACGGGACANCGGGCAGCGAGG

| | |
|---|---|
| Sequence 23 | cMhvSF110a12 |

CGAGGTACCGGAGACAGGTGCAGTCCCTCACCTGTGAAGTGGATGCCCTTAAAGGAACCAATGAG
TCCCTGGAACGCCAGATGCGTGAAATGGAAGAGAACTTTGCCGTTGAAGCTGCTAACTACCAAGA
CACTATTGGCCGCCTGCAGGATGAGATTCAGAATATGAAGGAGGAAATGGCTCGTCACCTTCGTG
AATACCAAGACCTGCTCAATGTTAAGATGGCCCTTGACATTGAGATTGCCACCTACAGGAAGCTGC
TGGAAGGCGAGGAGAGCAGGATTTCTCTGCCTCTTCCAAACTTTTCCTCCCTGAACCTGAGGGAAA
CTAATCTGGATTCACTCCCTCTGGTTGATACCCACTCAAAAAGGACACTTCTGATTAAGACGGTTG
AAACTAGAGATGGACAGGTTATCAACGAAACTTCTCAGCATCACGATGACCTTGAATAAAAATTG
CACACACTCAGTGCAGCAATATATTACCAGCAAGAATAAAAAAGAAATCCATATCTTAAAAGAAA
CAGCTNTCAAAGTGCCTTTCTGCAGTTTTTTCAGGAGCCGCAAGATAAGATTTGGGAATANGGAAT
AAAGCTCTAGTTTCTTAACAACCGACACTCCTNCAAAGATTTANTAAAAAAAAGTTNACCAACATT
AATCTNATTTTACAAAAAAAAATCTTTGGNGCCTANAAATACCTTTTTAAAAAAGGNNTTTTTGAA
ATANCTATTNAAAACTGGTTTTTTTTTTTTTCCAAGCAAGTNTTCCAACCCAACTTGGGTTCTGGCT
TAAAAAAAANTTTTGGGAAAAAAAAAAAAAAAAAA

| | |
|---|---|
| Sequence 24 | cMhvSF112h10 |

CGAGGTACCGGAGACAGGTGCAGTCCCTCACCTGTGAAGTGGATGCCCTTAAAGGAACCAATGAG
TCCCTGGAACGCCAGATGCGTGAAATGGAAGAGAACTTTGCCGTTGAAGCTGCTAACTACCAAGA
CACTATTGGCCGCCTGCAGGATGAGATTCATAATATGAAGGAGGAAATGGCTCGTCACCTTCGTGA
ATACCAAGACCTGCTCAATGTTAAGATGGCCCTTGACATTGAGATTGCCACCTACAGGAAGCTGCT
GGAAGGCGAGGAGAGCAGGATTTCTCTGCCTCTTCCAAACTTTTCCTCCCTGAACCTGAGGGAAAC
TAATCTGGATTCACTCCCTCTGGTTGATACCCACTCAAAAAGGACACTTCTGATTAAGACGGTTGA
AACTAGAGATGGACAGGTTATCAACGAAACTTCTCANCATCACGATGACCTTGAATAAAAATTGC
ACACACTCAGTGCAGCAATATATTACCANCAAGAATAAAAAAGAAATCCATATCTTAAAAGAAAC
AGCTTTCAAGTGCCTTTTCTGCAGTTTTTTCAAGGAGCCGCAAGATANGATTTTGGAATAGGAATA
AAGCTTTTAGTTTTTTTAACAAACCCGACACTTCCTACAAGGAATTTAGAAAAAAAGGTTTTACCA
ACCATTAATCTTANGTTTTACANGAAAAAATCTTNGNGCTNAGAATTCTTTTTTAAAAAGGGTATT
TTTGGAATNCTNTTTAAAAAACCTGNTTTTTTTTTTTTTCCNGCAAGGTNTTCCAACCCAACTTTGG
GTTTTTGCTTTCAAANAAAAAAAAAAAA

| | |
|---|---|
| Sequence 25 | cMhvSF113c04 |

AGGTACTGTGGATATTTAAAATATCACAGTAACAAGATCATGCTTGTTCCTACAGTATTGCGGGCC
AGACACTTAAGTGAAAGCAGAAGTGTTTGGGTGACTTTCCTACTTAAAATTTTGGTCATATCATTT
CAAAACATTTGCATCTTGGTTGGCTGCATATGCTTTCCTATTGATCCCAAACCAAATCTTAGAATCA
CTTCATTTAAAATACTGAGCGGTATTGAATACTTCGAAGCAAGAACAAGGCAATGTGCAGCCCTCA
TTTATGAGAAAACCCTCAGGAAACTCCCAGGGTGATGCTTGGAGAAGCTGTGAGTTGAGCTGAAG
CTGGAGAACTTCCTCCAGAGCAAAGGGCTTANGAAAGGAAAAGAAGAACTCTTAAGCTGGGGTCT
GCTAACATCACTCCAGTTTAANATGGATCTTGGCAGAGAAGACATTGCCTTTGTTCCTCCTGGGAT
TGGGAAAAGAATGAATTTACTCTTCCGGGAAATNTTTCTTTTGGTCAACCCTGGTACCTTCGGGCC
CGCTTNTTNNAAACCTAAGTGGGANTCCCCCCCCGGGCTGGCCAGGGGAATTTCCAATTATCCAAA
GCCTTTTATTCGATTACCCCGCCGAACCNTCCAANGGGGGGGGGCC

| | |
|---|---|
| Sequence 26 | cMhvSF115b02 |

AGGTACAGGCTGACAGAGAAGATTCCCGAGAGTAAATCATCTTTCCAATCCAGAGGAACAAGCAT
GTCTCTCTGCCAAGATCCATCTAAACTGGAGTGATGTTAGCAGACCCAGCTTAGAGTTCTTCTTTCT
TTCTTAAGCCCTTTGCTCTGGAGGAAGTTCTCCAGCTTCAGCTCAACTCACAGCTTCTCCAAGCATC
ACCCTGGGAGTTTCCTGAGGGTTTTCTCATAAATGAGGGCTGCACATTGCCTGTTCTGCTTCGAAGT
ATTCAATACCGCTCAGTATTTTAAATGAAGTGATTCTAAGATTTGGTTTGGGATCAATAGGGAAAG
CATATGCAGCCAACCAAGATGCAAATGTTTTGAAATGATNTGACCAAAATTTTAAGTGGGGAAAA
GTCCCCCCAAACCTTNGTGTTTNAAAATAAANAGGGGGGGNGGCCCCNANTTTTTGNAAANNAAC
CAANCANNGATTNTTTGGGGGGGGGGTNANNTATAAAAAAAAAAAANCCCCNNGNNCNCNGGGG
TTAAAAAAAAAAAAAAAANNTANCCCCCCCCCCCCNCGGGGGGNNNGNNAANNNAANTTNNNAN
TTTTNNNNNCCCCCCCCCCCCCCCGGGGGGGGGGGGGGGGGGGGCCCCCCCNCTTTTTTTTT

| | |
|---|---|
| Sequence 27 | cMhvSF115c02 |

CCGGGCAGGTACACCTGTTGTCATTCAACAAGAAACCACTGGCACCCCACGCTCAGATACAGTGC
CCTCTCCCAGGGACCTGCAGTTTGTGGAAGTGACAGACGTGAAGGTCACCATCATGTGGACACCG
CCTGAGAGTGCAGTGACCGGCTACCGTGTGGATGTGATCCCCGTCAACCTGCCTGGCGAGCACGG
GCAGAGGCTGCCCATCAGCAGGAACACCTTTGCAGAAGTCACCGGGCTGTCCCCTGGGGTCACCT
ATTACTTCAAAGTCTTTGCAGTGAGCCATGGGAGGGAGAGCAAGCCTCTTGACTGCTCAACAGAC
AACCAAACTGGATGCTCCCACTAACCTCCAGTTTGGTCANTGAAACTGATTCTACTGCCCTGGGGG
GAGAAGGGACTTCCCNTGGGGCCAANAATAANANNATNCCGATTGGANNGGNGNTCTTTACNAN
AAGAGNCCCANCCCCAANCNNTCCCTGGNNCAAANNAAAAAAAAATAANNCCCCCCCCCNGNNG
GCNTGNAAANGAAATTTNNANTNTTNAANCNNNAACCNNNNCCCGGGGGGGGGGGGGGGGGGGG
GGNCCCCTTTTTTTTTTTTTTT

| | |
|---|---|
| Sequence 28 | cMhvSF115g01 |

AGGTACAGGCTGACAGAGAAGATTCCCGAGAGTAAATCATCTTTCCAATCCAGAGGAACAAGCAT
GTCTCTCTGCCAAGATCCATCTAAACTGGAGTGATGTTAGCAGACCCAGCTTAGAGTTCTTCTTTCT
TTCTTAAGCCCTTTGCTCTGGAGGAAGTTCTCCAGCTTCAGCTCAACTCACAGCTTCTCCAAGCATC
ACCCTGGGAGTTTCCTGAGGGTTTTCTCATAAATGAGGGCTGCACATTGCCTGTTCTGCTTCGAAGT
ATTCAATACCGCTCAGTATTTTAAATGAAGTGATTCTAAGATTTGGTTTGGGATCAATANGAAAGC
ATATGCAGCCCAACCAAGATGCAAATGTTTTGAAATGATNTGACCAAATTTTTAAGTAGGGAAAG
NTNNCCCCAAACNNTTGNGGTTTTTCAATTNAAGTGGNNGGGCCCCGCCCTNNTGNNAANAAAAA
AAAAACAAAAANNNTNGGGGGGGGGGNANATNATTAAAAAAAANAAAACNANCNNNNCCCNGG
NCCCCTAANAAAAAAAAAAAAAAAACCCCCCCCCCCNGGGGGGGNGGNNNNNAATNNNATTTTN
NNTNTTNNNNNCNNNNNGNNGNNGGGGGGGGGGGG

| | |
|---|---|
| Sequence 29 | cMhvSF117f12 |

AGGTACTTGGAAATGTGAGATGGCTGTGGTGCATTCCACTGGATGGGGTGGGAGTTGGGCTGACT
CGGAGTCTCAGTGATAAATACTTCGACAGGACCACTTGAGCTTGGATAGGTCTGTAAAGGTTGGCA
ATGCCACTCCCCAATGCCACGGCCATAGCAGTAGCACCGGTATCTGACACCATGCACATACTTCTC
CCATGAATCTCCAATTTGATAAAACGTCCCAGTCTCTGAATCCTGGCATTGGTCGACGGGATCACA
CTTCCACCTGCCCCGACCCTGACCGAAGCATGTACCTCGGCCGCTCTA

| | |
|---|---|
| Sequence 30 | cMhvSF117f12 |

CCTGTGTGAAAATTGTTTATCCCGCTCACAATTTCCACAACAANATTACGAGCCCGGGGAAGCCAT
AAAAGTTGTAAAAGCCCTGGGGGTGCCNTAAATTGAAGTGGAGCTAACCTCACANTTAAATTTGC
GGTTTGCGGCTTCANCTTGGCCCGCTTTTTCCANGNCGGGGGNAAAACCTTGTCCGGTGCCCCANC
CTGCAANTTAATTGAAATNNGGCCCAAACGCCCCGGGGGNAGAGGCGGGTTTGGGGTATTGGGGG
GGGTTTTNTCGGTT

| | |
|---|---|
| Sequence 31 | cMhvSF024d10 |

ATTGGAGCTCCCCGCGGTGGCGGCCGAGGTCAAGCTTTTTTTTTTTTTTTTTTTTTTTTNNNGNNNN
NTTTNTGCANNCTTNNNNANCCNCCNCNNCNAAANNGGNNGGGGNNCNTTTTNAAAAATNGNNN
NNNCANGNANGNANAAAGGNNNTTTGCNNNGNTTNNANANNGCGATNAANATANGNCCCCNCAT
CATTAAGCCNTNTNAGAANGGGGNNCATNAAAAGNNANGGGGGATTTTNTNTGGNGGGCCNCCC
NAAANNAANTTNAAGNNGGNGANTTNAAAAAANTTNTGANACANCCNGGAGACTGGACNTTNTT
NNANCCNGNCNNTGNTGCTTTTAAGGGATTTACTANCNAAGAAAAANANNCCCTGNTTCGGGACA
AAAAAATGCTCTTTTTAACATTCA

| | |
|---|---|
| Sequence 32 | cMhvSF024e05 |

NATGGAATCCTGTTGGCNCATGATNAANTAACCCTTACNGTTCAGGGTTCCTGGAACTTNTACCNG
GGCCACTCTGACGGGCCTNACCACAGGTGCCCCCTACNACATCATANGTGGANGCNCTGAANAGA
CCANCTGAAGGCANTANTGGTTCGGGAACNAGGNGTGTTACCGNTGGGCAACTCTGGCTTGAACC
AACCTACGGATGACTCGGGCTTTG

| | |
|---|---|
| Sequence 33 | cMhvSF055a10 |

GAATNGGAGCTCCACGCGCGGTGGCGGGCCGAGGTACACAGTCAGTGTGGTTTGCCTTGCACGAT
GATATGGAGAGCCAGCCCCTGATTGGAACCCAGTCCACAGCTATTCCTGCACCAACTGACCTGAA
GTTCACTCAGGTCACACCCACAAGCCTGAGCGCCCAGTGGACACCACCCAATGTTCAGCTCACTGG
ATATCGAGTGCGGGTGACCCCCAAGGAGAAGACCGGACCAATGAAAGAAATCAACCTTGCTCCTG
ACAGCTCATCNCGTGGTTGTATCANGACTTATGGTGGGCCACCAAATATGAAGTGAGTGTCTATGC
TCTTAAANGGCACTTTGACAAGCAGACCAGCTNAAGGTGGTGNCAACACTCTGGAGAAATGTAAG
CCACCCAAGAAAGGCTTGNGTNACAGATGCTCTTGAGAACCACNATCNCCATTNNCTTGGAGAAN
CAAGGACTGGNACNATTNATTGGCTTTCCAAGGTGGTTCCCGTTCCAGGCCATGGGCCCGACTTCC
AATTCCGGNGAACCCNTTTAGGCCNGAATGNTGGGAAGCTTCACCATTACAGGGTTTACCANCCA
GGCCTNTGACTTACAAGATTTACCTGTACCTTGGGCCGGTNTTANAANTTGNGGGATCCCCCGGGC
CTGCAGGGAATTNTTNTCAAGNNTTTTNGTTACNGTNNACCTTTAAGGG

| | |
|---|---|
| Sequence 34 | cMhvSF055e04 |

TCGACTACTATAGGGGCGAATTGGAGCTCCCCGCGGTGGCGGCCGAGGTACATTTGTTTATTTAAA
GCACAGGAAATGAATAAAATGCCACCTAAAAAGTATCTGCAATGAATAAATTATTTCCAGTGAAG
CACTGCAGATCCACACACACCAGTCTGCTAACCTTTACCAAGGCCATGTCCGGTGGGCTTGTGCTT
GTCCCAGTTGACTCTTCCTTGAGACCTTTCCCTTCTGTGCAATGACCACAGCATTAGAGACCAGTCC
TGCATGCGCTGGCCTTCCTCGTAGGCATGGCAGACCACGTGGATGAGCAGTGGGCTGGCATGCAG
TAGGCTTCAACAAATGGCACTTCACTGTTTCCAGTGACCCTGAAATGTTTTATGTAAGTGGGGCCT
GGGCTTTAAAGAAAAGAGCCAGGGTTCCTCAGGCTGGGCCCCTTCACTGAGGCACAGCTCCAGGA
AATACTGGTCTCAGGAGCCAGCAACTTGTCCAGGAGTTTTGAGCCCTCAGTTGAAGGAAAATGGC
CACGTGGGTGTCCTTGCAGGCAACAGTGATGTCGGTGATGGTGACAAGTANCCAGCCTAAGGAAG
GCCAATCCCACCTTGGGTGGGAATGCAAGGGCACCTAGTCCTGCTTGGAANGGGCTNGGAAGGTT
GGGGA

| | |
|---|---|
| Sequence 35 | cMhvSF094a10 |

CGGCCGAGGTACGGAGCAATCGANGAGGCATAACCACACNNGGGGTGGCTATAGGGCTGGAAAA
CGCTGAAGATGACTGCTGACACNGAGGCCAAGGATNGNAATACAGCCAGCTTGGNAAAGACATN
AAAGCAGGAGNCNCTACAAGCGAGCNGCNGCACTAAGAAACACCCAACACCNCCANGNGCCTGG
ACAGGAGGCCCCCAGCAGAAACATGCACGCATAAGCTTCAAGCNCACTCCCTAGGATGGATGANA
GANGGGCNCCCAANNAANGGANGCCCACCAGGACCCACCAGNCAGGGCCCCANG

| | |
|---|---|
| Sequence 36 | cMhvSF100c07 |

TCCCCGCGGTGGCGGCCGAGGACCCTGTTTTANCGGANACANCAAACCCACACGAGCATGCGCGC
TCCNACANGANAGNGGGCCNAACACTAANCTGAAAGCANAAGTGCGCGGGCCGACTGACCNACN
CAANAAGAAGNTCANANANNACNACANCNTTGGCATCATGGTGGGCGGCAAAGGCTTTNCTANCC
GANNCCAAACCNGNTGTGAAAAACNCTTCATGACAAAAGACGTGAGCCGGGGTCGANANCCTGN
AAGCACAACAGGCNANAGAGCGANCNCNCATGTATGANAGAACCCTCGAGGACACTCCCAGGGG
AGATGCGCCGNNNAANCTGGGAGCAGAGCAGNAGCNGGCAAACGCCCNNCAGAGCAAAGGGCTT
AAGAAAGAAA

| | |
|---|---|
| Sequence 37 | cMhvSF100f12 |

GGCCTCTAAANTGCTGNTGGTCATTNGGCTGAGTCANAAAGCCACAAATGTCTGCTGCTGTGATAT
ATAGCTTGTCAGCTTTACAAAGCGGGCCTACGCCATTCTNATCAAGAAGAATGGTTGNCACAGTAT
TNGNGAACTGCACCNCAGGTGGAGTGCTAACA

| | |
|---|---|
| Sequence 38 | cMhvSF100f12 |

CACACCATCTTTGTCTAGAATACCCTTGGGGGTGGGATCTAGCACCTGGGATTTGCTGCTGAGNTT
ATCTTTGGGAGG

| | |
|---|---|
| Sequence 39 | cMhvSF113e08 |

CGCGGTGGCGGCCGAGGTACTATGANCCNAACACCAANNGCTNCNCTGNATTGTGNGNTGGAGGT
TGAGNTGGNAACNANANCNAANTCGGATCACATAAAGAATGTANAAAAGGTTTGCCGCTCCTGTG
CTNGCCAAACCCGGNGNTATTANTGNGATGGGAACCTAAANNNNNNTGGTCAACATCATNTACCT
TTTGAACAATAANGANTCCCACATCGTCANCTTNTCTATGGTGAANCTCCGGGTGTANATTCCCTN
GCNCTGTATGATTTCATGCTTGGGATTTACACTCAGAACTTCGGGAGGGAACATCCTGNTGTATGA
CCTATNCCTNTGGGGCNAATGTGTGTGTGGACNCTCTCTCTCTGACTCCANNCNTNTTNTGGACAA
TTCTNNNAATGANGGGGTAANACTTAACCACTNCNGGTNNTNATCTAAACATTTCTATNTAACCAA
ANTCNCTNNTGGAGNTTTGTGCNATGCCTGTTGCNNGCTATATGTAANAGNCTAGAATAATAANTG
CAAAATGGATATGGCTAACTAAANATNCTTTCAAGGTTGNGTTTCNTTTTTTT

| | |
|---|---|
| Sequence 40 | cMhvSF115f01 |

AGGTACAAGCTGNCANNTAATATTNCNNANAGTNNNTNNTGNTNNNAAATCAGCANGAACNNNC
NNGNTNCNATNNNAATANNNANCNANACTGAAGNGAAGTAAAGCATCACCCANCNCACTAGTCC
ATCTNTATTTCTTACCNCCTTAACTCTAAGAGGAACTTTTTCAGCGGGTATCTCACCATCACGGAGT
TGAATCCACATTACCNTNCNNAGAGGTCCTGAGGNGGAAATCATAGGAAAAGGCTGAACATTGCC
TGTTCTGCTTCTAACAATCACAATACNGTTNNGNGGNNNTAAAAGANNGCGAGGNNNATATTTAG
CNTTGNGCNCNATNTGAAATCNANTANNGNGCAACAACCATNCCCCNCGTTTTTTAATNGAAATG
ACNACCTGCTNNGCGGGCCCNAAAAGTGNCNCGNAACATTTNGCGGTTTTCCANCGAAAANANTT
NGNNCCCCNCTTTTCCCCNNNGGAAGCGCCNTAANGAGGGGCCNNGGGGNGGTTTTTTNAANNAN
AGGGCCCCCCCNCTNCCGGGGGGGGGGTGANAAAAAANAAANANTAAACCCCCCCCCCCCCCCG
GGGGGGGGNTTTTAATNNAAAAAAAAACNCCCCCC

| | |
|---|---|
| Sequence 41 | cMhvSF023f04 |

CTCCACCGCGGTGGCCGGCCGANGTACACTCCNTGGCCATACCCTGGAATTCTTCCCTTAACA

| | |
|---|---|
| Sequence 42 | cMhvSF024a08 |

GCTCCCCGCGGTGGCGGCCGAGGTACAAGCTGTTTTTTTTTTTTTT

| | |
|---|---|
| Sequence 43 | cMhvSF087a01 |

NCTTAGGGCGAATTGGAGCTCCCCGCGGTGGCGGCCGAGGTACCACATCTNAAATGCTCTCCAGN
GTTCTGAGNCTATTATGGGAGGANCNNCCTTTGAG

| | |
|---|---|
| Sequence 44 | cMhvSF090b01 |

AGCTCCACCGCGGTGGCGGCCGAGGTACAAGCTTTTTTTTTTTTTTTTTTTTTTTTTTGTTTTTTTTTT
TTTTTTTTTTT

| | |
|---|---|
| Sequence 45 | cMhvSF093b01 |

GGCGGCCGCCCGGGCAGGTCA

| | |
|---|---|
| Sequence 46 | cMhvSF093e03 |

CTCCACCGCGGTGGCGGCCGAGGTACAAG

| | |
|---|---|
| Sequence 47 | cMhvSF100d07 |

GGCGAATTGGAGCTCCCCGCGGTGGC

| | |
|---|---|
| Sequence 48 | cMhvSF108g05 |

CCGGGCAGGTACAAGCTTTTTTTTTTTTTTTTTTTTTCTTTTTTTTTCTTTTTTTTTTTTTTTTTTTTTTT
TTTT

| | |
|---|---|
| Sequence 49 | cMhvSE006c08a2 |

GATTGGAGCTCCCCGCGGTGGCGGCCGAGGTACTTAAGTGACTACCAGGATTGGTCTTAGGCACTT
AGGAAAATGTAGAGTCTGTTATATAGCTAATAAATGTAGGATCTGTTAAATATCTGACACAGCTGA
TATAACTTGTGCTTATACACATCTGTTAGAATGAATTGGAACATCTTGCTGTTCAGGTTGTAAGCTA
CACAAATCACCCGTTGCCTAGATTCAGTTTCCATGCGCCTTAAAACTTGAATATTTAGGTATTTGTT
TATAAAAATACAACTTATTATAACTCAGAGTGTAAGGATACATGAGCCAACTGTGCAATGGTTGTT
AACAATCTAGGATGGTGCAAGGAAAAAAATTAACAGCCAAATATAAGAAAAGAGATTTGGGGCT
GTTGGATTCAGCAAGGAATGAGCATGGCTTGATTCAGTAAAAGATCATTTTTCTAAAGATTAGTGC
CTCATTCAATATGTCTCTTCTCAATCTCCTGCCTCT

| | |
|---|---|
| Sequence 50 | cMhvSE043b11a3 |

GCGACACGGGACAACACNGAGTTTTTACGCCCGGGGGAGACGCTCNACACNCACACCNAAGACGC
NCNGTGTTGTATNNAGGGTGTGCAGCGGGCCACAGGGCACCTTGNTGTAGAACAGGCCCAACAGA
CNCGCCTNGGGGAGAGTTGTGCCTACNGGAAGAGNNGGCATAGAGGCACATTGTGGGGNCGTTTG
CCCGTCTGGCACA

| | |
|---|---|
| Sequence 51 | cMhvSE043f10a3 |

NGGGGTGGGGCCGGGCCCGGAAAGGGTACCTTGGNAGCCANGGGATTANCGNTGGGGCCANCGA
ACCCCATTCATTCCAGGTTNGGGGGTTAAAAAACNTAAACCTTGGTCTTCAACGNACNGGTCNTAA
AACCCCAAGCTTCAACGGTTTCCCCTTAATTAAGTTGGGGGTGGGAAAACAAATTNCCAACCGCCT
TTGGGTGGAAAATTCTTGCTTTCACCAAAATGGGANTAGGGGAAAAGAAGCCCCGACCATTCGGA
AAGGGAATCAAAAAAAAAGCCGGAACGTTCGGCNTTATTGGAAACCGCCTTTGGGGCCCCGGCCC
ACCAAAGGCCCAAGTTTTATTCCCTTGTTGGGGNTAAAACTTTTTTTCTTGGAACAACCCTTTTCTT
GGCTTTTAAAAAAACCCCCCAAAAAAANGGGGTCCAAGAAAAAGGGGAATCCGNTTGGAANGGG
CCCCCCGCNTTTTTTCAACCNGGGTCCTTGGGTTANTTTTCGGTTACCCCNTTCGGGGGCCCGGGTT
TTCTTTAAAAAAACCTAAAGTTGGGGGAATTCCCCCCCCCCNGGGGGCCTTTGGCCAAGGGGAAA
ANTTTTCCCAAATTNTTTCCAAAAAGNCCTTTTANTTCCGGAATTANCCCCGGTTCGGAAAANCCN
TTCTNAAANGGGGGGGGGGGGGGN

| | |
|---|---|
| Sequence 52 | cMhvSE043c09a3 |

GGAACCTCCCNCCCGCGGGGGGGGCGGGCCGAAGGGTTNCCCAGCCCCCCACCCAGCCAGCCCCC
TTTGGACCANGCCTTTAAAATTNGGGGGATTGAAGTGGTTAAGGGGCCTTNTCCCTTTAAGCCATT
AAAGGGGGAAAAGGAACNGGNTTATTTAAAGCCTTGGAAGAAAAGAAATTTGGAAAAGGAAAAG
NAAATTGGGGAGGCCCCCCCAAAAGGAAGNAAATTAGGCCATTAAAATTTAACCAANGGAAAGG
GGGAAAAACCATTGGAAAAGGAAAACCAAAGGCCNCCTTTTAAAAGNAATATTTTAAACCTTTTT
CAAGNCCCTTTTCTTCCCATTTTTCNTTTGGAATGGTCTTNAAATGGAAGGGCCAAAAAAANTAAA
CCTTGGGGGCCAANGGGGACCCCNCCCCAAAGNAATTGGAAAANAAAAAGTTTTAAAATTTNAAA
AATGGGTCCNCCAAATTGGGAAAAATTTTGGAAGGTTGGCCCAATTTAAATTACCAAACCTTGGTT
TGGACCTTGGGACCTTTTTTCCCCAAAAAAACCCCCCCGGGTTGGGAATCCGGGTTTAAGNAAGNT
ATTTCAATTCCAAATGGGTTTANCCCCCGGGANGGGGGAATTTTTTNGGTTTTTCCTGGGCCCTTCA
ATTTTTATTTAAACCTTCCACCTTTTCCCATTGGTTANTTTTTTTNGGGNCCCAGGTTTNTTAGGTTA
NCCCCNTTTGGGGGNCCCGGCTTTTTTTTAANAAAACCTTTAGGGTNGGGGGAATTCCCCCCCCCN
GGGGGCCNTTTGCCAAGGGGGNAAAATTTTCCCNNANTNTTTTCAAAGGNCCNTTTTNTTTCGGGA
TTTTACCCCCCGGTTCCGGAAACCCNTTCNAAANGGGGGGGGGGGGGG

| | |
|---|---|
| Sequence 53 | cMhvSE043b09a3 |

AAAAAANTTTTTAANCCAAAGCCTTTANTTNTAGGCCAGGGGGACTTTAACCCCNTTTTTCCCTTCT
TGGCATTAAATGGAAANTTAAACTTAGGAAAAATTAACTTTTGGCAAAGGGGAGGAGGCCCAAAA
AGCTTTAAGGACCCCCCGGAAAACCAGGGACCGAAGCTTACCCTTAAAGGAAACCAGGCTTAAAA
AAGGGAAGCCACCACCCCCCGTCTTANTTGTTAGGCCAAAAAAATAAGTGGGGGGAAAGGAATTT
TATTAAGGGGTTAGGGAAGGGGCGGAACCAAAAACCTTACCCCGGAAGCCCCTTGGGTGGAANTA
AGGCCTTGGGTTTGGTCCCCAAAAGNAATTAGGGAAATTCNTTTAAGTTTCAAACCTTTTTTAAAA
ANTTTTTGGCCCCCACCCAAGNAAACCCCCTTCTTTTANTCCCCCCCANTAACCTTAAGGTTTTAAT
TTTANTCCGGAAAAACCCCATTCNGGGGCNCCTTAACNTTCAATTTTCCAAACCCCAAAATTAAGG
CCCCCNTTGGGCCCCGGTTACCCCTTNNGGGCCCCGGCTTTTNTTAAGNAAACCTTAAAGTTGGGG
GAATTCCCCCCCCCNGGGGGCCTTTGGCAAAGGGGAAAATTTTCCCGNAATTATTTCAAAAGGCCT
TTTANTTCCGGAATTACCCCCGGTTCCGGAACCCCTTCCNAAANGGGGGGGGGGGGGGCCCCCCG
GGGTTACCCCCCCAAGCCTTTTTTTGGTNTTCCCCC

| | |
|---|---|
| Sequence 54 | cMhvSE043c08a3 |

GGTTTNAACCCGTTTTNAAATGNGGACTTACTTNGTTTNGCCTGNAATNGGGAANCCTCCCCCCGC
CGGTGGGGCCNGGCCCGAAAGGTTACTTTTTTTCAAGNTTAAAATTTAAAATAAAAATGGGCCAA
NTTTGGGAAGGGANGGGGGANCAAGAANAAAGGGAACATTGGGGGGGAAGTTGAAGAACCCAA
AACAAAGGGAATCAATGGAATGGAAGAACCCAAGAAACTTTCCCTTAAGAAAGGAAGTTGGGCC
CCCGTTTGTGGAAGCCCTTGGAAAAAAGAATCCCCCTTGTAAGCCGACACCTTGGAAGCCAAGNA
AGCCTTCCCTGGTGGCCCCTCTTTTCCGGTCCCTTGGGCCCTCAACCGCCTGGTCCTGGGTTTGGGG
CTTTTTCCCCCGGAATCCCCGGTCCGTTCCCAATCCTTCTGGTTTGGGTCCCCTTGGTTTGGGTTTGG
TTTGGTTTTGGTGGGGTTTTTTTTGGAAGAAATGGGGGGGGGGTTTTTCCGGCTTCTTTTGGTTTTG
GCCCCCAANGGGTTTCCTTGGCCAAAAAAAAACCGTTNGCCTTGGAAGAAAATTTTCCTTAAGTNG
GGGAAGGGCCACCCCTTAAAAAGTTCCAAGTTGGAAAGGTNGGGGAATTAAACCTTGGGGTTNAC
CCCTTTNGGGGCCCCGCNTTCNTTAAANAAAACCTTAAAGGTGGGGGAATTNCCCCCCCCCGGGG
GGCCTTTGGCCAANGGNAAATTTTTCCGAAATTANTTCCAAAAGCCNTTTAATTCCGNAAATAANC
CCCGGTNCCGAAACCCCTTCNNAAAGGGGGGGGGGGGGGGGC

| | |
|---|---|
| Sequence 55 | cMhvSE043c07a3 |

CATTTTTCCAAAAACCCATNTTCACCTTTCAAGTTTTTCCCATTNGGGTTAAACCAATTTGGCGGGG
GGCCTTTCCNTTGGGCTTACCCAATNAAAGTTCGGCCAATTAAGTTTGGAACTGGTNGGGAAATTT
TCTTCNAAATCCTTCTTTTAACATTCTTTGGAAGCCTTGGGGTCCTGGTTTTTTATTACCACCCAAA
AACCAAAAACTAAAAATCATTCCTTGTTTACTTTTAAAACCAACCACCAAAAGTTTCCCCATTCCA
AGAAAATGGCCCTTATTATTTTGGAAGAAAACCCACCAACCGGTTGGCCCTTTCATTAAGGGGGGT
TCCAAGCCGGNAAGGGGTTAAAAAAGCCNTTCTTTCCGGGCCAAGCCGCCCGGGCTTTGGAAAAC
TTTCCCTTCCCCAAGGGGTCNTTNGGGGTAACCCTTCGGGGGCCCGGCNTTCNTTAANAAAACCTA
AGGTNGGGGAATTCCCCCCCCCGGGGGCCTTGGGCAAAGGGNAAAATTTTTCCGGAATTANTTCN
AAAAGGCCTTTTAATTCNGAAATTANCCCGGGTCCGNAAACCCTTCCGGAAAGGGGGGGGGGGGG
GGGC

| | |
|---|---|
| Sequence 56 | cMhvSE043h02a3 |

GGTTTTTNGGGGTAAAAAANAAGGGCCNGGGGGGGTAAAAGAATTTGCCCGANGTTTCCCTTTTT
ACCTTTTTTTTTTAAACCCTTTTCCCTTAATTGAAGCCAATGCCCTGGTGGTTGGGGGGTTTTGGAC
CAAGTGGAAGGGGTAAATAAATTGGACCTTGGGTTTGGGTTTGAATTGGGTAAGNAATATTTTGG
GGGCCTGGTTTAAAATTTGGTCAAGTTTCCAAGTTGGTTTTTTAAAATCCTTGGACCGCCAAGGGC
CTTTAATTTGCCGGGAAGGGAAGAAAATGGGTTTTTTCCAATTGGTTTAACCTTTAATTACCTTAAA
CCATTTTAAGGTTTCTTTTCTTAATTAAGGGGGGTNGGAATAAGNAATTTNGGGGTTCCCCAAATT
TTGGGGGGTTGGTTGGAAAGGGGAAGTTTCCAAGTTTTAATTAATTGGTTTTTNGGGGGGGAATTT
TTTTTTTTAAGGGGTTAAGGTTGGGGGGTTGGTTTTGGAAGNCCTTTTGGAAAACCGNCCTTTTTCC
TTTTAAAATAACCCCTTTCGGGGCCCCGGCCTTCCTTAANAAAAACCTTAAGGTNGGGGAATTCCC
CCCCCCNGGGGGCCTTGGCCAAGGGNAAAATTTTCCGAATTATTCCAAAAGNCCTTTTAATTCGGG
AATTANCCCCGGTNCCGAACCCCTTCCNAAANGGGGGGGGGGGGGCCCCCCCGGGTAACCCCCCA
AANCCTTTTTTTGGGTTTCCC

| | |
|---|---|
| Sequence 57 | cMhvSE043h01a3 |

AGGAAAATGTAAAGTCTGTTATATAGCTAATAAATGTAGGATCTGTTAAATATCTGACACAGCTGA
TATAACTTGTGCTTATACACATCTGTTAGAATGAATTGGAACATCTTGCTGTTCAGGTTGTAAGCTA
CACAAATCACCCGTTGCCTAGATTCAGTTTCCATGCGCCTTAAAACTTGAATATTTAGGTATTTGTT
TATAAAAATACAACTTATTATAACTCAGAGTGTAAGGATACATGAGCCAACTGTGCAATGGTTGTT
AACAATCTAGGATGGTGCAAGGAAAAAAATTAACAGCCAAATATAAGAAAAGAGATTTGGGGCT
GTTGGATTCAGCAAGGAATGAGCATGGCTTGATTCAGTAAAAGATCATTTTTCTAAAGATTAGTGC
CTCATTCAATATGTCTCTTCTCAATCTCCTGCCTCTTTTTTTAAATGCCTCTTTCTACACATATATTT
GCACATAATCTTAGAATATGATTCTGT

| | |
|---|---|
| Sequence 58 | cMhvSE043h09a3 |

CCNTTTCTTTGGGGATTCCCNAAAAAAAAAAAAAAAAATCCAGCAAGCCACAAAATGGCGAANGG
GGGTTTCTTTGGAATATTAAAGCCGCCCCGCNATTACCGTGGGAATNGGGGGTTCAACAATCCCTT
GGTTNAAATCAATGGAACTTCCCACGGCCAAAGGAACAACAAGGGAAGTTCNTTCCAAATTGGGA
ATGGCCCCTTCCCAAGGGGTATTCNTTTTTTCCAACTTCCTTTGGCCAAGGAATTTTTTTTTTTTAAT
GGTCCAAAATNCTTCTTTTTCCCGGAACCCAATTTCCTTCCCTTCNAAAAACCTTGGGTAACCCCTT
CNGGGGCCCCGGCTTTCCTTAAGAAAACCCTTAAGGTTGGGGAATTCCCCCCCCCCGGGGGGCCTT
GGCCAAGGGGNAAAATTTTTCCGGAATTAATTTCCAAAAAGCCCTTTTAANTTCCGGGNAATTTAA
CCCCCGGTTCCCGGNAACCCCCTTCCCGGAAAGGGGGGGGGGGGGGGGGGGCCCCCCCCGGGGGT
TANCCCCCCCAAAGNCCTTTTTTTTTTTGGGGTTTTTCCCCCCCTTTTTTTTAAAGGTTGGGGAAGG
GGGGGGGGTTTTTAAAAAAATTTTTGGGCCCGNCCCGNCCCTTTTTGGGGGCCGGGTTTAAAAAAT
TTCAAATTTGGGGGGTTNCCAATTTAAAGGGCCTTTGGG

| | |
|---|---|
| Sequence 59 | cMhvSE010e07a3 |

GCCGAAATTGGANCCTCCACCCGCGGTGGGCGGCCCGAAGGTACCAGCCGGCTTCATGGGAACAT
CAAAGTTCCCCGGCTTGGGAAGCCAAGGAAGAATTGGCCCACCTTACCCGCAGCCTGGCTTCCGA
GGGGACAAGGGAAAGAATCACCTTACCAACCAAATTTGTTCTGGCCTCCCAAGGGTCCTTCTTGAN
GGCAAGCAAGGCTTCTGGGGGCCTTTCTGCTTGTCCTTTGGGAGGGGTGGTTCTTTCTTGGGGTAA
GAAGGGATGGGGAAAGGGAAAGGGGACCCTTTACCCCCCCGGGCTCTTCTCCTTGACCCTACCCA
ATTAAAAAAA

| | |
|---|---|
| Sequence 60 | cMhvSE052c02a3 |

AGGTACAGAATCATATTCTAAGATTATGTGCAAATATATGTGTAGAAAGAGGCATTTAAAAAAAG
AGGCAGGAGATTGAGAAGAGACATATTGAATGAGGCACTAATCTTTAGAAAAATGATCTTTTACT
GAATCAAGCCATGCTCATTCCTTGCTGAATCCAACAGCCCCAAATCTCTTTTCTTATATTTGGCTGT
TAATTTTTTTCCTTGCACCATCCTAGATTGTTAACAACCATTGCACAGTTGGCTCATGTATCCTTAC
ACTCTGAGTTATAATAAGTTGTATTTTTATAAACAAATACCTAAATATTCAAGTTTTAAGGCGCAT
GGAAACTGAATCTAGGCAACGGGTGATTTGTGTAGCTTACAACCTGAACAGCAAGATGTTCCAA

| | |
|---|---|
| Sequence 61 | cMhvSE035d05a3 |

AGGGCGAATTGGAGCTCCCCGCGGTGGCGGCCGAGGTACTCCACGAGGAAACTACAATTCCAGGA
ACAGATTTGAAACTCTCCTACTTGAGTTCCAGAGCTGCAGGGTATAAGTCAGTTCTCAAGATCACC
ATGACCCAGTCTATTATTCCATTTAATTTAATGAAGGTTCATCTTATGGTAGCTGTAGTAGGAAGA
CTCTTCCAAAAGTGGTTTCCTGCCTCACCAAACTTGGCCTATACTTTCATATGGGATAAAACAGAT
GCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCCGTC
CAACATGGGTGGCTGGACATTAGATAAACATCGCGTGCTGGATGTACCTCGGCCGCTCTAGAACTA
GTGGA

| | |
|---|---|
| Sequence 62 | cMhvSE00605a2 |

GGCTAATTGGAGCTCCCCGCGGTGGCGGCCGAGGTATCTCCGGGGTGGCGCTGGGGTTGGCTCCAT
GACCAAGATCTATGGGGGACGTCAGAGAAACGGCGTCATGCCCAGCCACTTCAGCCGAGGCTCCA
AGAGTGTGGCCCGCCGGGTCCTCCAAGCCCTGGAGGGGCTGAAAATGGTGGAAAAGGACCAAGAT
GGCGGCCGAGGTCGGTAATTGATAATCTGGCACCCTGCAAGGCTAGAATGGCGATCAAACATTTT
CACTGGCTGAGACTCTCCTTCCATACTCCAGTGATAAACTGCATTATCCGTAACAAGAAGCAACCC
GTATTCAAAGAGATCCATTTCCAAAAGGTGACATCATCAGTCATGGTATGAGCCTTCATTTTACTTT
TCATTTCAATGGTTAAAAATCTGAAGAGTTTTNCCANCTTTCAAGTGCAATTTACTTTGCTAAGCCT
GGATTCATGATGGCGCCTGTCTTGGCTTGAAAATTGGGTCTT

| | |
|---|---|
| Sequence 63 | cMhvSE001e03a3 |

AGGTACACTACCTNANANTGNTTCCACNGNCNNGNCNCNNTGCTNNANNGNANGANGGNCNNTA
TNCTGTGTTTATNGCNTNGANGNTAAANGNGANAGCCNGNANTAAANGNATNCNTGNCTTTNGAN
CTATGAANCTCATNNCAAANNGATCTANNGNAANANCNNTGANGGGGNGNCCTGTNNNCNTGTN
CACCTACCTNTATGGAAAGGTNTGNTGGTNTCTTNAATTANACATGNNANTAGATGCCTGCTGGAT
AATATATAAACAATAAAAACAACTTTCACTTCTTCCTATTGTAATCGTGTGCCATGGATCTGATCTG
TACCT

| | |
|---|---|
| Sequence 64 | cMhvSE035c06a3 |

CGAGGTCGCAGCAGCTGGGGAGGAGCCAAAGCCTCGGCGCTCACCTAAGCCGCAGGGAGATACA
CCCAACTGGGAGATGAGGAAACAGCAACCCAGAGAGGAGAACTAACCCACACAGGATCATTTCGT
GAAGGAGCAAGGCTGAAGAACCAGACCTGGACTTTCTTAGGACAAACTTACTGCAGCTTGAAGGA
GCCAACCATGGATTTGAGGCGTGTGAAGGAATATTTCTCCTGGCTCTACTATCAATACCAAATCAT
TAGCTGCTGTGCTGTTTTAGAGCCCTGGGAGCGATCTATGTTTAACACCATCTTACTAACCATTATT
GCTATGGTGGGTATACACTGCCTATGTCTTTATTCCAATCCACATTCCCTGGCTTGGGAATTTTTCT
CAAAAATA

| | |
|---|---|
| Sequence 65 | cMhvSE044f03a3 |

CCGGGAGGCTCCCAGGCGCCCGGCGCAGTGGGAAGCTCGCAGCAGCTGGGGAGGAGCCAAAGCC
TCGGCGCTCACCTAAGCCGCAGGGAGATACACCCAACTGGGAGATGAGGAAACAGCAACCCAGA
GAGGAGAACTAACCCACACAGGATCATTTCGTGAAGGAGCAAGGCTGAAGAACCAGACCTGGACT
TTCTTAGGACAAACTTACTGCAGCTTGAAGGAGCCAACCATGGATTTGAGGCGTGTGAAGGAATA
TTTCTCCTGGCTCTACTATCAATACCAAATCATTAGCTGCTGTGCTGTTTTAGAGCCCTGGGAGCGA
TCTATGTTTAACACCATCTTACTAACCATTATTGCTATGGTGGTATACACTGCCTATGTCTTTATTCC
AATCCACATTCGCCTGGCTTGGGAATTTTTCTCAAAAA

| | |
|---|---|
| Sequence 66 | cMhvSE001c02a3 |

TAGGGCGAATTGGAGCTCCCCGCGGTGGCGGCCGAGGTACTCGAACANCATNCNGCAGCTGNTNN
ACAANTTCCCTCCTGACCANCTNACAAGCTNACGAGCGCCGTNNTGGTCTGGGCCCAAANGCTNT
NCACACCCNCTNACCTTTGATGTAAACAATCCCNTGNNTNTGGACTATG

| | |
|---|---|
| Sequence 67 | cMhvSE001f04a3 |

CCGGGCAGGTACCACGTGNACCACCACCGNTACCTGGGCGGNGACNGGCTGGACGTGGACGTNCC
CACACNTNTGGAGGGCTGGTTNTTCTGNACNCCCNCCCGCAAGCTGATATGGCTGGTGCTGCAGCC
CTTCTTNTACTNACTA

| | |
|---|---|
| Sequence 68 | cMhvSE001f04a3 |

ACGTACCNANCTTTTGTTNCCTTAAGNGAGGGTTAATNGCGCNCTTGGNGTAATCATGGNNANAN
CTGTNTACTGGAANTCATGACNNTGTCTGGGCTGCAAANAAGCANTGCCCNTGTGATCATTTN

| | |
|---|---|
| Sequence 69 | cMhvSE041c01a1 |

GCGAATTGGAGCTCCACCCGCGGTGGCGGCCGCCCGTGGAGGGTCAATCATGGAGATGAGCCCAA
CAAAGCACAGATTATCGATAGGGAAATTCACATCGTCAGTGTCAAACTGGAACCCTTCAGGAAAC
TGTTCATCTGGCAGAAAGAGGTGGCAGAAACCTAGGACTCGTTCTCCGAGGCCCCCCAGCTCCAA
ATAGGCGTTCTGAAAGGCGTCTTTCAGCTCCTCATCCAGGGGCTGCTCCTTGCCGTGGAGGAGGAT
AGAGCTGCAACGGTCTAGGATCCTTTCTGGGGCGCCCTTCATCACCAACAGGTGTTGGGGCTCCGA
TGTGTTGGGGTTCTTATGAATAGACAACTGGTACCTCGGCCGCCCGGGCAGGTACTTTTATCTTAA
AAGGGTGGTAGTTTTCCCTAAAATACTTATTATGTAAGGGTCATTAGACAAATGTCTTGAAGTAGA
CATGGAATTTATGAATGGTTCTTTATCATTTCTCTTCCCCCTTTTTGGCATCCTGGCTTGCCTCCAGT
TTTAGGTCCTTTAGTTTGCTTCTGTAAGCAACGGGAACACCTGCTGAGGGGGCTCTTTCCCTCATGT
ATACTTCAAGTAAGATCAAGAATCTTTTGTGAAATTATAGAAATTTACTATGTAAATGCTTGATGG
AATTTTTTCCT

| | |
|---|---|
| Sequence 70 | cMhvSE035e02a3 |

GGACCTTGTAGGGCACATACTTCCTGTAGATATGGCCCACCCTGGAGCAGGGGATGTCCTCCATGC
GGCCCCCACACATCCACACCTTGAAGGAGATTTCATACTGCTCCCCTCCCCAGATCTCCAAGCCTG
GGTCATACCCGCCGAGTTCCCAGAACCACTTCCGATCCACGGCGAACAGTCCACCGGCCATCACG
GGAGACTCAAATGGGTCGCTGGGGTCAGCTTTCTGCAGTTCTGGAGGGATCGGGATCCGCTTGTAG
TACCT

| | |
|---|---|
| Sequence 71 | cMhvSE043b06a3 |

TGGGGCNGGGCCCGAAAGGTACCTTATTGTGGAACTTTTCATTTGGATTGCCCCCCAGGGAACACC
AAGAAGAACTTTTTTCCAAAAAAACATTGGAATTACCAGGGGGGAACATTCTTCAANGGCTTGGA
ACTGGTGGCTGGTCCTGGAATTGGTTGGCTTGCCTTGGGTGGTTTGGGGTGGAAAATTTGGAAAAA
GCCTTGGGTTATTCTTCCAAAGAAAATTGGGGGCCAAGAACCCCCGNAAGAAAGCCATGCCCCTTT
CTTGGGCCTTTAACACAACTGGGGGTGGTGGAAAAACCAAACCTAAATTTGGTCCGGGTGGTTTTA
AACCAAAAAAATTGGGGAATTTTCCCACCTTGGAAGGCCCGCCCCCTTACCAAGCCCAGGGAAAA
GAAAGAAATATTTGNAAGGGGAAAAATTTGGGTTTAAAAGGGGAAAAGTTCCAAGGCCACCTTTT
TACCAATTTTTAAAAGAAAAAAAAATTTNGGGCNTTTACCCAAAACCCCCCCCGGAACCCACCNA
AGTTTAAGCCCAATTTTTTTGGTTGGNCCCCAAAAATTTTTTCCTTNGGGGTTTTGGGGGAAAATTG
GGGGGTTGGNAACCCAAAACCAATTGGNCCTTGGGGGAAAANCCCCAAAANGGGGCCAATTTGG
GTTTTAAANAAAACCCCTTTGGCCCCCCCGGGGGGGGCCCGGGGGNCCCCGGCNTTTTCTTTTAAA
NAAAAACCCTTAAAGGGTTGGGGGGAAATTCCCCCCCCCCCCGGGGGGNCCTTTTGGCCNAAGGG
GGAAAAAATTTTTNCCCNAAATTTANTTTCCNAAAAAGNCCNTTTTAATTTCCGGNAATTTANCCC
CCCGGTTTCCGGNAAACCCCCTTTCCNAAAANGGGGGGGGGGGGGGGGGCCCCC

| | |
|---|---|
| Sequence 72 | cMhvSE043h03a3 |

AGGGTGGCAAAAAAAAAAAGGGCCGTTTTGCCNTCAACAAATTGGTANCCCGAGAANTACNCCNT
CAACATTCACAAGCGCTTCCATGGAGTGGGCTTCAAGAAACCGTGCACCTCGGGCACCTCAAAGA
GATTCGGAAATTTGCCATGAAGGAGATGGGAACTCCAGATGTGCGCATTGACACCAGGCTTCAAC
AAAGCTTGTCTGGGGCCAAAGGAAATAAGGGAATGTGCCATTACCGAATCCCGTGTGCCGGCTGT
CCAGAAAACGTAATGAGGGATGAAAGATTCACCCAAATAAGCTATATTACTTTTGGTTACCTTATG
NTACCTTCGGCCCGCTCTAGAAACTTAGGTGGGATCCCCCGGGCCTGCAGGGAAATTCCGATATTC
AAGGCTTATCGATACCGTCGACCTTCNAGGGGGGGGCCCCGGTAC

| | |
|---|---|
| Sequence 73 | cMhvSE045g08a3 |

GGCNNATTGGAGCTCCCCGCGGTGGCGGCCGAGGTACCATTTGTGGTGCCCAAGTTTAAGTTATCT
TACATTCAACCCAGGACACAAGAAACTCCTTCACATCTGGAAGAACTTGAAGGATCTGCCAGAGC
ATCTTTTGGAGATCGAAAGGTAGAACTTTCCAGTTCATCCCAGCACGAACCTAGCTATGATGTGTA
TAACCCATTCTATATGTATCAGCACATTTCACCTGATTTGAGTCGACGCTTTCCTCCCCGTTCAGAA
GTGACGAGACTGTATGGATCGGTTTGTGATTTAAGGACGAACAAACTTCCCGGTTCCCCTGGGCTA
AGCAAATCTATGTTTGATCTTACAAACTCATCTCAGCGATTCATCCAGAGACATGATTCATTGTCC
AGTGTACCT

| | |
|---|---|
| Sequence 74 | cMhvSE030f02a3 |

AGGGCNAATTGGAGCTCCCCGCGGTGGCGGCCGAGGTACCACCAGAGGACACGGATAATCTTCAT
ATCTGATTCTCCTGCGGTGCGTGTGCCCTGACAGAAGAAGTTGTATTTGCCTTCCCATACTCCTGTT
ACTAACTCACAGAACATATACAGAGACAGCAGTGTGAGTCCAAGGTTATACACCACTAAAATCCC
CCGGCAAGAGAATGGCTGTTTATTCCTCATGTATTTTGGTCCCAGCCATACAATTAGTAAATATAT
GACAGAGCAGATAAATGTGGGTATATAATTGTCCAGAAGAAACCATCCTTTTACTCTAGTATCTCG
AGGGCCTAGCAATGCCTTGAAATAGGTACCT

| | |
|---|---|
| Sequence 75 | cMhvSE033d07a1 |

AGGTGTCGCCGCCGCGAAGGGAGCCGCCGCCATGTCTGCGCATCTGCAATGGATGGTCGTGCGGA
ACTGCTCCAGCTTCCTGATCAAGAGGAATAAGCAGACCTACAGCACTGAGCCCAATAACTTGAAG
GCCCGCAATTCCTTCCGCTACAACGGACTGATTCACCGCAAGACTGTGGGCGTGGAGCCGGCAGC
CGACGGCAAAGGTGTCGTGGTGGTCATTAAGCGGAGATCCGGTGAGTTTTGTCTGGTTTGGGCCAG
AGAGCGGCCCCTTTCCCGGGTCTGGGAAGCTGTGATTTTTTACTGTCAGGCAGGGAAGAGACGGTA
ACTGCCATCGCGGCGGGCCATCCCTGGGCGCCAGGGGTGTTTGGTCTGGGGTTACCTGCCCGGG

| | |
|---|---|
| Sequence 76 | cMhvSE043g02a3 |

GCGGCCGAGGTACTGNNAGGGNNAAAANNAGCTGNNNGGNNGNCANAAGTGCNTCTNCTTAAGG
ACCNNNNCCTGCTGGNATANAGNACNNAAACCTANNACCNTGGANTGNNGANTANCNTNANNGG
ANTACGGNCAAANGNNGGCCTGCGGCTGCTGAACTACCATTACTTCACTGGTGTCAGATGGGGAG
ACGNNGGCACGTAATGGGCATANNCNTCCTTNNNGGCNAATCTGCAAGCGTGGAAGGCANCNTGT
NACTGANGCCTTCNACTTNCACTTNTAACCTTGGAGCTNACTGNTTNCTGCCTNTGGGGNTTTTNT
NAAGAAACCNACCCACTGTGATCAATATTGGAGANAAANTGNACATTCTTGGGCTGAANACNGGC
CTCNNACACTGNTNACACTNGNCTNTGANNCNNCAGTACCT

| | |
|---|---|
| Sequence 77 | cMhvSE030b01a3 |

NAATTGGAGCTCCCCGCGGTGGCGGCCGATGTACATNTNTCNGNNANGGNCNGNTGNAGNAANAC
CNTANCAATCCTATCCATNCCGNTGACNNTGNGNGGGGGNNCAAAACCCAANTGCTGNTGCCTCT
NCCNNGCCNTNANTGNAACACTCAGCGAAANTCATGGTTCATAANTGAAACNTGAATTCCTCTAG
ACTCTGCAATACTGCACTCTTAACAAAAATCAAATGAAAACAAGACGTGTCTGCCACAGGTCTCA
GGGTAACAGATGCCCTGTCCACTGAGAGCGGCAGTTCTGCAGTCAGAGTTCTTTGATCAGCCCTGG
ACCCATTTATCACATGGGGGAGGAA

| | |
|---|---|
| Sequence 78 | cMhvSE040a01a3 |

ACTCCCCGCGGTGGCGGCCGCCCGNGCAGGTACAAAGCTTTTTTTTTTTTTTTTTTTTTTTTNNNAA
TTTTTTTTTTTTTTTTTTTTTTTTTTTTTTTTTTTTTTTTTTTTTTTTTTTTTTTTTTTTTTTTTTTTTTTTT
TNAAAAAAAAAAAAAAANNNTTTTTTTTTTTTTTTTTAAAAAAAAAAAAAAAAAAAACCCCCCNC
AAAAAAAAAAAAAAAAANNCCCCCCCCNAAAAAAAAAAAAAAAAAAAAANNNNCNACNCCCCC
CNNNGGGGGGGGGGGGGNNCCNNNNCNNNNNTTTNAAAAAAAAAAAAAAAAAAAANACCCCCN
ANAAAAAAAAAAAAAAANANNNNNNNNNNNNNNNNNANNNNNNNNNAANAAANANAANNNAN
AAAAAAAAAAAANANNAAAAAAANANANAANAAAANAAAANAAANNAAAAAAAAAAANANAA
NAAAAAAAAAAAAAAAAANNAAAAAAAANANAANAANNAAAAAAANAAANAAAAAAAAAANA
AAAAANAAAAAAAAAAAANNNAAAANAANNAAAAAANNAANAAAANANAANAANNANNANAA
ANNAAANNAAAANANAAAAAAAAAAAAAAANNAAAAAAAAAAAAAAAAAN

| | |
|---|---|
| Sequence 79 | cMhvSE006a10a2 |

CCGGGCAGGTACTACCCAAGTGTTACAGGCTCTGCATAGGTCCTCAAACACTTTAAAGGACACGA
ACCATCAAATTCAAAAGAGTAGTGTTTGTTCTATCAGTTCTGAATGTCCACAGGGAGAGGCAACTA
GATTTATGTGGAAAAAGTGCTGTTTGAAGGAGCTGTGTTTTATTTCGAAGTGAAATGACTTTGGGA
ACCAGAACATTTCTGCAGATGTCTGAATATCAAGAACCTATCTCTAAAAGGCATTTATCAGGAAAT
GTTCGCTCACTCCAAGTGCTTTTTAAAAATTCAACATATGGCAATGTTTTAATTTTTGTGCTTTCAA
GAGGTAACTAAATCGATAGGAAGCTGAGGGAAGATCATTCCATTATGGACTTTCTTGTTTGGGTGC
AAGACACTATCCACAGCATTGAAATCTATAATCTCATAAAAGATTCTTATAAACATATACCATATT
TCTC

| | |
|---|---|
| Sequence 80 | cMhvSE045d10a3 |

GATTGGAGCTCCCCGCGGTGGCGGCCGAGGTACTTGCTGGTCTCAAATTTCCACAAGGAGATATCA
ATGGTGATACCACGTTCACGCTCAGCTTTCAGTTTATCCAAGACCCAGGCATACTTGAAGGAGCCC
TTTCCCATCTCAGCAGCCTCCTTCTCAAATTTTTCAATGGTTCTTTTGTCGATGCCACCGCATTTATA
GATCAGATGGCCAGTAGTGGTGGACTTGCCCGAATCTACGTGTCCAATGACGACAATGTTGATATG
AGTCTTTTCCTTTCCCATTTTGGCTTTTAGGGGTAGTTTTCACGACACCTGTGTTCTGGCGGCACCT
GCCCGGGCGGCCGAGGTACTACCTGAAGGAGCTTCAGCTGCCCCTGAAGAAGGAATGAGTAGCGA
CAGTGACATTGAATGTGACACTGAGAATGAGGAGCAGGAAGAGCATACCAGTGTGGGCGGGTTTC
ACGAC

| | |
|---|---|
| Sequence 81 | cMhvSE011g01a3 |

ATTGGAGCTCCCCGCGGTGGCGGCCGAGGTATTANACCGNCGNGAGACAGGTTAATTNTACCCTA
CTGATGATGTGTTGTTGCCATGGTAATCCTGCTCACTACCTCTN

| | |
|---|---|
| Sequence 82 | cMhvSE011g01a3 |

TGCTGTTTCCTGAACTATACCAGTGGNGGAACACTTGAACAAANTGNNTACCT

| | |
|---|---|
| Sequence 83 | cMhvSE045h07a3 |

GCNAATTGGAGCTCCCCGCGGTGGCGGCCGATGTANAACTAGNGNATANNCCGGNCTGTATGAAT
ATTATATNANNCTNATNCATACCATTTANCNCAANGNGGGGCCCNNNNCCANCNTTTTNTTTNNTN
NNCNNAAGGAANANTGAACNCTAAGGAATACATCATGGTAAGATTCTNTCCTACTGTGTCAGCGA
GCGCTGCTGCCGGTCTANATTGCCATGTCCCAACAACAGCAAAGCCACCCTCCCTCCTGCTTCTTC
CAGGATTGCTCTTTAAAGGGACCAGAGTGACATACTGATGCCTACTGAGGCATCTGAGATGCACTG
TGTTGGAGGTTAGCCTCAATGCCAGCCTCTGGTTGTCTAGGTGAGTGACATCACCATAAAATCACA
TTGTGTACCT

| | |
|---|---|
| Sequence 84 | cMhvSE023d03a1 |

CTATAGGGCGAATTGGAGCTCCCCGCGGTGGCGGCCGAGGTACAAGCACGGTTGGCATGGCCTTT
CCAAAGGTCTTCCACTAGAGTCTAGAGAAANCTAAATATAGTCATCCACAAACTGGA

| | |
|---|---|
| Sequence 85 | cMhvSE011b04a3 |

TGGAGCTCCCCGCGGTGGCGGCCGAGGTACTCCATTTATATAAAATTCTAGAGCAGGCAAAACTAT
AGTCACAGAAAGTTGACCACTGATTGTTTGGGGCTGGCAGTTGGGGTATGATTGACCACAAAAGG
GCCTGTAGGAACTTTTAGGGTGACAGAAATGTTCTATATATTGAAGTTGTTTTTAGTTACATGGAT
GTAGCATTTGTCAATAATCGGCTAACTGGACATTTAAAATGGTTCCATTTTCTCACATGTAAATTAT
ACCTCAAAGTTGATCCAAANAAAAAAAAAAAAAAAAAAAAANGTTTNGNCCNNCCCGGGGGGNC
CNTTNAAAAAANGGGGACCCCCCCNNCCNGGGNAATTTNANTNNANCNTTTTNAAACCCCGNNCC
CCCGGGGGGG

| | |
|---|---|
| Sequence 86 | cMhvSE032e09 |

AGGTACCAGTTATCCACTCACTGACTTAGGTGCCTCCACTAGAATTCTCAGCACGTTTTTGCAGAA
CCTGGGCAACAAGAGCGAAACCCCATCTCAAAACCACAACAACAACAACAGGACAACAGAGATT
GGACGACCNGATNGGGNAAAAGCCAANNCANACANGCGTGAANGGCCAGGTACCGGNAAAGTAG
GCACAAGGGNAGCNTCTGCTCAGTGTCGCTACANGGGGGATCTCTCAAGGACTTNACAAACGNGG
NCCACATCCTTCNTAGNGGGAAAGATTACTTGGTTCTCATTNAATGGATCCCTTTGTTTTNGGGNN
CCTACACCTTCNCCCCAATGNTTCNCTTTTCTTNCTTGGTANTCCCNTNNCTNTNCCNAAACTTGGG
CCCAATTTTTAATTTTTAATTTTTTAAACCT

| | |
|---|---|
| Sequence 87 | cMhvSE043f05 |

CGAGGTACAGTCCAGTCCTTGGAGATCGACCTGGACTCCATGAGAAATCTTGAAGGCCAGCTTGG
AGAACAGCCTGAGGGAGGTGGAGGCCCGCTCGCCCTACAGATGGAGCAGCTCAACGGGATCCTGC
TGCACCTTGAAGTTCAAAAGCTGGCACAGACCCGGGCAGAGGGACAAGCCGCCAGGCCCAGGAGT
ATGAGGCCCTGCTGAACATCAAGGTCAAGCTGGAGGCTGAGATCGCCACCTTCCGCCCGCCCTGCT
GGAAAGATGGCGAGGACTTTAATCTTGGTGATGCCCTTGGACAAGCAAGCAACTCCATTGCCAAA
CCATTCCAAAAAGACCACCCACCCCGCCCGGATAGGTGGGATGGGCAAAAGTGGTGTCTTGAAGA
ACCAANTGACCACCCAAAGTTCTTGANGCATTAAACCCAGCANAAGCANGGGTACCTTNGGCCGC
TTCTAAAAACTAGTGGGATCCCCCCGGGCTTGCCANGGAATTTCGATATCAAAGCCTTATCGAATA
CCCGTCCGACCCTCNAAGGGGGGGGGCCCCGGTACCCCAACTTTTTTG

| | |
|---|---|
| Sequence 88 | cMhvSE001a09 |

ACGNACTAATNCTGACTGTNAANGNGACGCNTNACGANCNTTCNCNCCTTNTGGGTCNNAANCAG
GANGAGTTNGATNANNCATNACANAGNTAANNGNTTNGNGGCGNANNAGNATCCNTAACAAAGN
TACTTNTAGNACGTCTGATGGNACCTCTNCCTATCTTTAACAAGCNGATTCCNCCNACNGNTGGAT
TGNTAANNCACTNTTATCGGANACCTGAGCNNTTTTAGGACGGGCCCGAGACAAGCTTTTGTTACC
TTACTGANGANGTGNTGGNGCCCTGGGNATANTGNTNAGTACCTGCCCGGGC

| | |
|---|---|
| Sequence 89 | cMhvSE001d12 |

NCNNGGCNNGTACACGGGAAACNATTNATTCNNGNCTNANGGGGANTTNCCTTANCGGATACTAN
ACCCATACNTTTNANGGCTATGANCACAGACANGTNAGATNCCATGCNNCCTGGGCCANGATCTT
CCNCNANTAGTTNCCTGCTTAAGCAAATAGAATTTCTTANGGGGCAGATNCCAAAANCACCGATN
ATTGGAAAGCAAACACCNACACTGCCANCTTCCCTCCCAGGACTCCTGCCAAGGTTTCCANTACCT
AACGNCGCTCTAAAANTAGTGAATCCCCCNGGCTGCAATGAATTCGATATNAAGCTTATCAATACC
CNTCATACCTANGAT

| | |
|---|---|
| Sequence 90 | cMhvSE001e12 |

AGGTACATGGANNNATTGGCTTNTNACCNGNTGCTCNNCCNGACCATTGNTNGCNGGCNNNTGGN
CATNNACNAAGCCANAANNAAANNTCTGNCACAAAANCGAAATCTNCCNATNTACATTACNAATA
CGNTAAANCNCACCAAGGNGTGAAGGCGATANTGCAGGAACTGCAATGGACCCCTGGNTGGAAC
CCTATCATAGGGACAAGGATGGCTTCCTGGGAACTCCGAGNGGANGGANGACTGCTNNNTNANNC
NAGCACANNCANGATGAAGANNTNTTNATTCTTTAAGANCCTNGNNATTGAACTTNACACTGATC
TGTACCTCNCC

| | |
|---|---|
| Sequence 91 | cMhvSE001h10 |

GATTGGAGCTCCCCGCGGTGGCGGCCCGNCNNGCCANGTACATAAGCNAATATGCCCATTGGGGN
CCTGGGCACTANNNNGTCTNTTTTNGGCANAANNAATGANNCTGTGAACGTGGCCCNTGATGCCT
AATATCCCACAACNACTGTGCCTAT

| | |
|---|---|
| Sequence 92 | cMhvSE007f03 |

ACGTNCCAGGGGCTGTGNATNNACTACCTNNCATAGANCNCCGCCCTCATTCAGCNCAAANTNTA
NGACTTCTTGNTCAANCTGAGNNCNNCATNNATANNNACCNNNCNNTTNNNNGANNNANNNANT
CNCNANNTANTGANAANANTCTTTNTNTNCACCNTNANNNTTANGNTNNTCANNNNCTNTCAAGA
CAANTACGNGNNCAATATNAGGNNTNCTAATNTTNGGGGCNCGATNTTNNTANNTNCNANTCTGG
CTATATAACTNNCCACATGACTGNTANNNNACTTCAATCGTTCAAGAATTATATGANCCTATGACC
NCAATNAATNCCATGTACNTCTNANGCNTNNCAACTACNNGANCGNNNGGCCTGNAANAANTCTA
TATNAACCTTANCTNAANNTTAAACCTCCACNGGGGGCCNTCATCCCAATTTNTGTTCCTNTAATG
AAGGTTAATTGCNCCCTTGGCG

| | |
|---|---|
| Sequence 93 | cMhvSE010c02 |

AGGGCNAATTGGAGCTCCCCGCGGTGGCGGCCGAGGTACAAGCTTTTTTTTTTTTTTTTTTTTTTTTT
TTTTTNAAAAAANCCCCCNTTTTNAATTTTTNNNCCNNTTTTNNNNNNNAAAANNNAAANCCCNTN
NTTTTTTTTNNNNCCCCNGNCCCNNTTTAAAAANCNTTTTTNNGGGNNCCNGGGGGGGGGGNCCCC
CNCNNTTNGNAAAAAANCCCCNNNGGGGGNNNNCCCCCCNTTNNNANNCCNNNNANNNCCNNNA
AAANGNTNAAAAAANNCCCCCNNNTTTTNGGGGGGNCCCCNNGNNTTTTAAAAAAAAAACCCCN
GGGGCCCCCCNAAAGGGGNNTTTAAAAAAANCCCCCNTTTTTTNCCCCCNGGGGGGGGGNNCCCC
CCAAAAAANCCCNTTTTTTTT

| | |
|---|---|
| Sequence 94 | cMhvSE011f07 |

ACAAAGATGNTCCNNNNGTNCCNAATACNCTTNAAAGAANNNGANGGANTTTNCNTGANCTATNT
ATCANNCGCCTGNCANNTAANNAGGCCCNNAAGATGCTATTACCANGCNTAGANCGAACCATNTG
TATNAGAAANCCNNGNCCTATCNCANNGAATNTNGGCCNATNTTCCTGGGCNGTTCNNGNACNAG
AGGANCNCCCNGGANNNGGNAATCNTNNNTNCAGNTTATCNANACCNGCNNCNTCGCNGGNGGG
CCNNNANNCNAGCCTTCGTNCCNTTTAANGANGGNNCNTAGCNCNCTNNTNCNNNTNATGNNCAN
NGCNNNTNCCNGTCNANAANTTNTGGATCNNNCGGGNTGNNNGANTNCGCTCTTGGCCTNATCAN
TNCCATAGACCTTTCT

| | |
|---|---|
| Sequence 95 | cMhvSE015e06 |

AGGTCTAATCTACAAGCGTGGTTATGGCAAAATCAATAAGAAGCGAATTGCTTTGACAGATAACG
CTTTGATTGCTCGATCTCTTGGTAAATACGGCATCATCTGCATGGAGGATTTGATTCATGAGNATCT
ATACTGTTTGGAAAACGCCTTTNAAAAGGAGGGCCAAAATAACCTTTCCTGTTGGGGCCCCCTTTT
CAAAAAATTTGGTTCTTTNNTTCCACCGTAGNGGTNGGNAATTGGAAAAGAAAAAAAANAGNAAC
CCCAACCCCCCATTTTTTNTTGNTTNNGAAAAATNGTTGGGGAAGAAAANTGGCCTTNGGGCCAA
AACCATGNGGGGTAGGGGGGAACCCCAAGNAATTCCNAAAACCCAAGGGGGCCTTTTAANTTTTA
AGNAAAAAGGAAAATTGNGAAAACCTTTAAAANGGGGTTGNTTCCTTTANCCCAATTNGAAATTT
TATTTTTNNTTTTCNTTAAAAGNNCCTNGGGGGTNTTGGGGTTTTAAAANTNAAAAAACCCANGG

| | |
|---|---|
| Sequence 96 | cMhvSE016b08 |

GCTCATCAACACCTCTGACTTTGAGTTTTTTCGTGAAGGTGGGAATGTTTAGCTCGGGAGAGTTGA
TTTATAAGAAAAAGACACGCTTACTGAAGGCCTCCAATGGAAGAGTCAAGTGGGGAGAGACTATG
ATTTTTCCACTTATACAGAGTGAAAAAGAAATTGTTTTTCTCATTAAGCTTTACAGTCGAAGCTCTG
TAAGAAGAAAACACTTTGTGGGCCAGGTTAGTAGGAGTTTTTATCCTTCCTTATATTTTTTCTATGC
ATTTAAACAGTCAGTTAACAAAGGGAATACANGATAATATTAAAGTCAAATAGAAGNACCTCGGC
CGCCTCTAGAACTAGTGGAT

| | |
|---|---|
| Sequence 97 | cMhvSE017e06 |

AGGTACNTATCGATACCCACATNCNNNNTNNNNACNANNNANTANNNTAGAGTATCTATGNNNTT
CCCTGACTNNATGNNNNGTGAANGTGNNNACATCCTNCCGCNNTNATNAANGGATACTNTGACTN
CCTNCTCCTCACTGAGGTGCCTCATNCTACCCGGGNGTNCCTNTGCCANCCTNCCTGGNACATNTG
CTNGNACCTGCCCNATGCCAGGATCATGGNACCAGGCNAGAGGNCACCCGTTNCTTCCTCCCNCA
TGTAGATAAATGGGTCCAGGG

| | |
|---|---|
| Sequence 98 | cMhvSE026f02 |

CTTAGGGCGAATTGGAGCTCCCCGCGGTGGCGGCCGAGGTCCCNTACNGACACTGGCCCNAGTAN
ACGGTGAGTNATGGNGNCANTTGNNTGGGANGAGTTCATAAATATGNTTGGNAGCTAAANCGCAT
GGNNTGATGCTCNTGAANNCTAATNCTNNTGGNTNNNTNCAGTCATGCCTANANANCCTGGTGNA
NTGGTGANATNANTACNCAGGGGTTTGGT

| | |
|---|---|
| Sequence 99 | cMhvSE043b12 |

NAATTGGCAGCTCCACCGCGGTGGCGGCCGAGGTACAGATCANNGTGGNTTNCCTNCNTTGNAAN
AATAATTTNGCTAAACCACNAAGTGTNNCGTGCATTGCTACTACNTTGGNTCTGNNTCCACAAAAN
AGNTTTGAACTCTGCTAACTCANANTCTTAAAAGAAATCTCCTGGTCTAATNGTATNATGAAAAAT
AANAACTATNANCCGACAATTGAGTT

| | |
|---|---|
| Sequence 100 | cMhvSE048g10 |

AGGTACAGAGNTGCCNANNANNNGGGNNCTNTNCTTGNANCACNNGANTNGNTNNCTNTAACAT
GGGGCTACTTACGNCTTCTTACNNGANCACTTGGNNANATTTNCCTTTGNNCTAATACNNNGNNAC
GTCATAGATGGTNTGGGACATANTCTTCCTCCCTTAGAATCGTGGGGGAGCGTGATGATGATCCAC
TANGTGTTAGCAATATGCCT

| | |
|---|---|
| Sequence 101 | cMhvSE052g11 |

ATAGGGCGAATTGGAGCTCCCCGCGGTGGCGGCCGAGGTACNNTTANANCTCCANGAGAAGTGAN
TNATNANANATANNTNCTATTANANNNCTGNNNNNNANCATNCTCNGNNGGTCCCANNCTNNNTG
NCGATNAGANNACTGAGGGNNNNTNAGAAANNNNCTATGCNTTATGCAATTGNTNTGTCNTNANN
NCTNNTCNTATCNACTATAGCNNTTNCTNGNNACATNACANTNCNNGCNNCAATCTNGANNNANT
GGATCNTCNGGCNNGCAGNAANTGCANATGNTNNTTATACNTNCNGCNGANNNAANAGNGGNNN
CNNGCTNNNNCCTATGNNANCNTTATATGNCGGNATNTNGCACACNGGTNCTANTAANNNTNATA
TNNATTTGCNGAANATGTACCT

| | |
|---|---|
| Sequence 102 | cMhvSE003f06 |

AATTGGAGCTCCCCGCGGTGGCGGC

| | |
|---|---|
| Sequence 103 | cMhvSE003g02 |

CNAATTGGAGCTCCCCGCGGTGGCGGCCCG

| | |
|---|---|
| Sequence 104 | cMhvSE011c06 |

GCNAATTGGAGCTCCACCGCGGTG

| | |
|---|---|
| Sequence 105 | cMhvSE011e07 |

CTCCACCGNGGTGGCGGCCGAGGTCNNNCAACATGGTGTTNA

| | |
|---|---|
| Sequence 106 | cMhvSE011f02 |

GAGCTCCCCGCGGTGGCGGC

| | |
|---|---|
| Sequence 107 | cMhvSE030e05 |

CTGATTGGAGCTCCCCGCGGTGGCGGCCGAGG

| | |
|---|---|
| Sequence 108 | cMhvSE030g08 |

NGATTGGAGCTCCCCGCGGTGGCGGCCGAGGTACCCAAAACAAGTGCTTAAAAAAAAAAAA

| | |
|---|---|
| Sequence 109 | cMhvSE035b08 |

TTGGAGCTCCCCGCGGTGGCGGCCGAGGTACNAGACCCAGAGGCGGCTGCTCTCTCCCCCCAGCT
NNGTAAGGNGCCTCCAAAAANAAATTTTTTTTTTTTTTTTTTCTNCTGGGGATGCA

| | |
|---|---|
| Sequence 110 | cMhvSE040g07 |

CTAATTGGAGCTCCACCGCGG

| | |
|---|---|
| Sequence 111 | cMhvSE010d06 |

GCTCCCCGCGGTGGCGGCCGAGGTACCACCATTGTAAGGAAACACTTTCAGAAATTCAGCTGGTTN
CTCCNAAANAAAAAA

| | |
|---|---|
| Sequence 112 | cMhvSE044h08 |

AGGTACCTTTNGACCCCATGGAAAAAAAATATCTAACGTNCAGAACTACCAAT

| | |
|---|---|
| Sequence 113 | cMhvSD003c05a1 |

ATTGGAGCTCCCCGCGGTGGCGGCCGAGGTACGGTGGGGCACCCAGGTAGTAATATGCAGGAAGT
AGAATTGGCAACAAAGGACACAGAATGAAATGGTGAGATGGCTAGCGGAAACATAGGGAGAATG
GCATCACAAAGGCAAAGGGGGGAAAGAATTTCAGTTTAGTGGATAGTCAACCAAGGCATTTCACT
TAGCAGTCAGGAATGAAAAAACGATACTGAATTTGAACATTAGGAAAGCTTGGTAAATTTCAAGA
GTATAATTTCTGCAAAGTTGGAACACAGTGAATAAAAAAGTGCTAAGAAATTGAGGACAATTGAA
AAGTTTAGCAAATGATAAGACAAAGCAGAAGAAGATAGTAGATAGTGAGGACAGCANAATCAAT
AGGAGGGTTTCTTGGGAAGGCCATCTTTGTTTTAAAGTTTATGGGGAGAGAACCAGTGTGCGAATG
GAAGTAGCTAGGGGGAGAAACTGAAAATGCTAGGAAGACTGGGTGTGGTGGCTCATGCTTGTAAG
TCTCAGCTGCTCAGAAGCCTGACGTANGAGAATTGCTTGACCCCANTAGTTTCGTGACCAGCCTGG
AATATANCCAGACCCTGTTTCCATAAAAAAAAAAAGCTAGGAAGGTAA

| | |
|---|---|
| Sequence 114 | cMhvSD003d03a1 |

TACTTTTAAACCAGGTGAGAAAAATTAAATTATGTATTCTAACAAAGTAATATGTGAGATTTTGCA
AATGATTTTATAGAAATACACAAAATAACTCTTTAGCTTGCTCTGAGCATTTTTTTCTTTTCTGATA
GCAACTTTTTAACGTTGTGGATCCACAGAACTTACTGCTTTGCTTTCTCTTTTGGGGTCATAATTCC
TCTCCCCTTGGAGTGTCCACTCCATGCATGTGCACTTAGGATGTGTGGCTGTGTGTGTGTTTGGGAA
CCCTCACGGACACATAAGGTTCTATTGTCATCAAGTAGAAAACCTATCTCATTATCATTATAATGT
CTTCAGATGCTTTCTAAGGTTCACCTCTTTTTTAACATTAGAAGTCAGTGAATGCAGCTTTCATTAT
AATTTTTAATACTTTAAAATGTTTTTGTATTANCTGCCANAATGCTCAGCAGCAAAAGTTATGACTC
ACTTCTAGCAAGTGTGGTAGTTCTTTGCTTNAAGCATTTGGGTTTCATGTAGCTTTTCTTCTTATTTT
TTCTTTGG

| | |
|---|---|
| Sequence 115 | cMhvSD090b03a1 |

GGGCAGGTACTTTTTTTTATTTTTTATTTTTTTTTATTTTTAGTAGAGATGGGGTTTCGCCATGTTGG
CCAGGATGGTCTCGATCTCCTGACCTTGTGATCCACATGCCTCGGCCTCCCAAAGTGCTGAGATTA
CAGGTGTGAGCCACCGCGCCCGGAAGGGGAAGGATCTCTTTATTCAAATACGCACATGCACGTGC
ACAGATACCTTGCATCTGTGAAAGGAAGCTAAGAAATCTGCAGTCGGCAGCTATTTGGAACTATG
GCTTATAAACTTATGTTTTTCAGGAGACAGAGAAACCAAGACTTGGGCCAGTCTTTGCAGTGACC

| | |
|---|---|
| Sequence 116 | cMhvSD090c03a1 |

CCGGGCAGGTACTTAAACACCAGGCGGACATTTCTCCAGGAAGCATTCCATAGCTGTCTCCTCCCC
CACCTTCCAAAGGTCACAGAGAACCCTGGGCCCACCTCTGTGGCTGCAGTCACTGTGCTGATTGTC
ATGTCTGTTTACTTGTATATTTCTTGGCTACCCTGTTAGCTGCACAGGGGAGAGACAGATCTGATTT
GATTTGGTATTGCTAGTGTGAGACATAGACCTTGGTGCTCAATATATGTTTGTGAAAAATCACAGA
AGAGGCCATAAACTGGGGGCAGAAAATCAAAAGCATTAGGTCAAAAGATATCAGAGGATTCACA

| | |
|---|---|
| Sequence 117 | cMhvSD090c05a1 |

AGGTACCAAAATTCTAACTTAGGGCTTTAGAGTTCCTGGATTCCAAGGGAATGCACTCTTACATAT
ACTACATCATGTGCTGCTCACCATCCATGTGGTGATGAGGAGCATTAGATAAGGAGCATTAGGTCC
ATGTAGCAGAACAGTAAACTGAAGCTCCGAACAGCGAAGGAGCTCACCCAAGAGAGCACAGGGC
TAGGATCAGGAA

| | |
|---|---|
| Sequence 118 | cMhvSD095a02a2 |

CCGGGCAGGTACAATTTATTGCAGACCCAGACACGAGAAGGTCAGAGAAAATCAGAGAAAGCAA
GCAAGTGAATTTGCCTTACTCTAGGACCCACACTTTGGTGATCACAGCTGGATGAAGAATGTCAGG
GGATGAATCGGAAGAAATGAAACTGGAAAGAGGAAGGAACCAAGTCTTGAAGGGCCTTGGAAGC
CATGTTAAGAAGGATGAATGAGAGGTAAAGAAGACGACATTGAGCTTTCTCACTTGGGCAGTTGG
CGGATGGCAGTTGGTGGATGGCAGTGGGTGGATGACTTTACTGAGGTAGGAAGCCTGAGNAGGAA
AAGCAGGTTTTGAGGGAGAGTTTGACTAATTGCAGTTTAAGACATGTCATGTCGGAAACATCATGT
ATCACACTGTCCCAGTAAGTAGTTTGAAGACAAAGATCTGGATCTCAAGAGAAGGAGTATGGGGC
TGAAGATNGCAATTATGGGAACTATTGCTACATTGGTTGGGTTATTAAAGACAAAAGAAGTTNGCT
TGAAATTTGCCAAGGGGAGAGTTTNACCAGANNGAGAAAACCAGGCCCCAGGATTAGNAGCTTCC
CAAAGGAACTTTNAAAAAGTTAAA

| | |
|---|---|
| Sequence 119 | cMhvSD095c02a2 |

GGGGCCATTGAGACTGCCATGGAAGACTTGAAAGGTCACGTAGCTGAGACTTCTGGAGAGACCAT
TCAAGGCTTNTGGCTCTTGACAAAGATAGACCACTGGAACAATGAGAAGGAGAGAATTCTACTGG
TCA

| | |
|---|---|
| Sequence 120 | cMhvSD095c04a2 |

AGGTACTGTGCTCAGCCAGGAGAGGCCCAGCATTGCTCAGTGGCTATGCTCCTGACGGATTCTGAT
GATCGATGTANACCTTCGGAGATCACTGATACCTAGCCACTTAATCTCGTTCCTCACAGCCAGAGA
ATATACGTAAGTAAATTGCAGAAGTGTTGGACTCAGGAGAGGCCAGTTAGTTTTGGGGCACCTCTC
TTACAGAGCTCTTTGGGTGGAAAGAAGAAGTGGTGAAATGACCTATGCTTCTGTTTCATCATGACA
GGGAAATCTGGAAGGGGAATTCAGTCTAGTGAATTTACTTAAAATATTAGCTGCANNAAACTAAT
TTACAGGGGAAAGCGGCTTTGTGACATTTTTAAGTGTAGAANGATCCANATGAGAAATGTGAATTT
CNTACCAGAAACTTTGGGGTAGTCCT

| | |
|---|---|
| Sequence 121 | cMhvSD095d04a2 |

AGGTACTTTTTGGTTACTACCTTTACAGACGGCATCAACATGGACCCTCACACCTGCACCTGAGCA
ATGTGGGACATTTGATTCCTCATGGTGACAGTTTCTTTCCCACCCCAAGCTCCAGGGAGACAGTAA
GCTTTCTCATCATTTCTCTGGGCTTGTGGGCAAACATTTTTTAGTCTATGGGAACAGGGAGCACTTC
CAGACTCTATTCTTCATGCAGGAATCTTAATTAAAACCTCTCCACCTCANATATGCCTGCAGCCAC
GTCCGTTGTCCCCAAACAGATATTAAAATCCAGCATTAGGACCACTTAGCCCTATTCCTATTTGAA
AGCCTCTTTGGGCAGCCATGATATCATTATTATTCTCCTTATTCTGGGATTGCTTTTTTACTTCATTT
CTTCTTCTTTTTAAAGTATTANGCTCTATTGAGATATAATTCAGATATCACACCAANTCACCTATTT
AAAAGTATACCAATTCAATGGGTTTCTTAGTATATTCACAGAGCTGGGCAACCATCACCACAAGCC
AATTTTTAAGAACATTTTTTCTTACCCTAAAAAAAAGAAACCCCCNGTACCCTGCCCGGGGCNGGC
CGNTTNTAAAAACTAAGTGGAATCCCCCCGGGGCTTGCAAGGGAATTCCGANNTTNAAGGCCTTN
TTNGAATACCCGGCCNACCCTCNNAGGGGGGGGGG

| | |
|---|---|
| Sequence 122 | cMhvSD095f01a2 |

CCGGGCAGGTACTTTAATACCTGTGATCAAGGTGTCTTTAAATAATTGCTTTCATCTGTGAATGGC
GAAATTACTAGCATAATAAGATTGCTGTAATATTGGTCAGCTTCTGGAGTAGATAGATAAAGAATT
GTGTAATCAGTTTGTGTCCCCAGCTGAGGGGATATTCCTTCTCTTCTCGTTTTATATTAATTGAATT
ATTTTTTAACTCCAAAAAGAAATACATACTTATTGTTACTAATTAAATAGTGCANGGTTATTCAAA
AGAAATCTTAATTTTTCTTTCACTACCTCCCTAAGGAAGGNTAACGTTCACTATTCAGTATCTTTTC
ATACTTTTTTCTTTGGTTCTACAGTAAACATAAAATAGCTATATATAGNGGCCCCTTTTAAATAAAA
ATGTGGATTGTGCAATNACAACAATTATTTTTATTCCTTTTNAAACACNTTGTTTCAAGGGGTTCTT
GGGGCC

| | |
|---|---|
| Sequence 123 | cMhvSD095f11a2 |

CCGGGCAGGTACCTGAGGTGACCCCAAAATTCATCCAAATATTCTATCCAAGAGCAGGCAAATGC
TACATGGGAAATCACAAAGAGGAGGAAAAAAAGAGAGAGAAGAGACAAANTGAAGCTTTGACAA
GCAGCTCAGCTGGGCCAGCCCCTTGGAAGGGAGCCAGCATTGGGAAAGCAGCANCAGCTC

| | |
|---|---|
| Sequence 124 | cMhvSD095g09a2 |

CGAATTGGAGCTCCCCGCGGTGGCGGCCGCCCGGGCAGGTACCATGGCACATATGTGAGGTTTTCT
TCAAAACAGATTGTGTTGCAGGAACTGAAACACCACCAAAAACAATCCCATTAAATGTGGGCAAA
GGGGCCGGGCCTGGTGGCTCACACCTGTAAGCCCAGCACGCCTGGCCCCCATATTCTTAACTACCA
AGCTGTATGCTCTCTGGGATCCTTCACAAAACATGAATGTCACTGCTCTGCTGTATGCCTCCAGTCT
CCCCATCTCTCCTCTCCTCCATCATCATACCTTTTCCAGCCTGTCCCTTGTGCAGTTCTTGGCTCACC
ATCTGAGTATCTATGAGACTGCTTAAAGTCTCTCTGCCTGGAATTAAAACTTGCAAATGAAAGCCTT

| | |
|---|---|
| Sequence 125 | cMhvSD095h03a2 |

GGCGGCCGAGGTACCTTTCTTTCCAGGCCATGGCAAAAAAAATCCAATTATGTCCGTCTTGAGTCT
GTGGNCTTGCTTCTTATGTAGNATTNCCTTTGTGAGCTGAANATTAATGCATGGATTCACCTCCTTC
AGCACATTTCATTTCAATTGTGAAGAAAAGATTCCAGGCACTGAATGTAAAATTGAACATGACATT
TTGACATTCCTTCTTCTGAGAGCTGGGTTGGTCTTAGTTGCTGTGAGGCTCTANACACCGACCATAC
AGGGCGTGGGGCTGCTCCTGGACATGAACATACTTACGAAGTTCTCCCCAATCCACTTTACCCCGN
CCCCGCGTACCTGCCCCGGGCGG

| | |
|---|---|
| Sequence 126 | cMhvSD084g12a1 |

CCGGGCAGGTACGCGGGGGCGCGACTTCCCTGGCCCGCCCCCTGCGGACCAGTGAACCTCGCCCG
AGGGCTCAATAAAGAAGATTTTTGCCCTCTTTTTCTCACCTCTCAGCCTTATTGATCCATGGTGCCC
TTCCATTGCCTTTCATTGGTGCCGAAACCCGGGAGGGGACACCTCCTAAGCCCCCCCAGAGGCTCA
GGGGGACTCCCCTCCTGGTCGGATCAGTCCTCTCCCTCAGTCAGGTCAGGCTTCTCCTCCACGGCC
ATCTGTCCATTTCGTCCGGTTACTTGCTACCAGGTCGCAGTTGCTGCAGCTACTCCAGT

| | |
|---|---|
| Sequence 127 | cMhvSD090c04a1 |

AGGTACTTTCCCAGAGGAACCATTCATCAAGCGGACACTCCTGCGGGGCTGGCCCACTCGACTCAC
GTGACCATCAGCACCTACCAGAACAAGTAAACACTGCCTCCCAGCTGCACATGCTAGGACAGCTC
TGAGTCCTGGCCTGCAGCAGCCACATTCAGGAGGGATATGAGGGAGTTGGCCCCTACCTCCTACGC
AAACCCCAGGGTTTATGTCCTTTACTGACTTCCACATTCCTTTGATGTCCCATGTATGTGACTGGTC
CCTCTGGACTTGCTTCTGGGGACATCATGAACCTGACTCTGTAGGATGTGGGGCATTGCCCAAATA
GAGA

| | |
|---|---|
| Sequence 128 | cMhvSD090g02a1 |

CCGCGGTGGCGGCCGAGGTACAGCCTGTGGAACTCTTGAAACATGGATTTTTTCCTAATAATTGAA
GACGGTTCAAGAAAATATCTTCTACAAGAAAATATGCAACTAGGAGTCCTGCAATGAACCGTTGTT
TGCTTTCTTCAATATCAATTATAATAATATTTTATCTTTAAAATCAGAATTTTACCGAAACAGTTTT
GTCATTTTATTATTTAACTGATGAGAAAAACTATATGTGATTTAGAGTTGCCATGAGTCCTGATTCA
AATCAGATTACTTTTCTTTTGCTAAAAACTTAGCGCAGTAGCCCACCTACAATCCTGCTTGCTTAAG
GGGAAATGGTACCTGCC

| | |
|---|---|
| Sequence 129 | cMhvSD090g04a1 |

CCCCGTAATACCGACCTCACTATAGGGCGAATTGGCAGCTCCACCGCGGTGGCGGCCGAGGTACC
CCAAACAAGTTTTCCTATTTTATTTTTATGCTTACAGATACTCAAATATTAACAATTTAATTAATCA
CCAGCTATTAAAATCATGAAAACATCATGAACACACACTACCGGTGTGGATCTCCACAGTGCTGA
GTTTTTAGATGACATTCCCTACACCCCTTCCTCTATGAAGAGTTTCACAAAAGACGTCTTTAGAAG
GTAAATCTAGCCTATGAAATATTTTAAGCAAAAGACAGAAAGAAGTCTCAAATGTATGTGGTGTA
TGTGGGGTGTGTGTGTGTGAGAGAGAGAGAGAGAAAGAGAGAGGGAAAGAAAGACACAGAGAC
AGAG

| | |
|---|---|
| Sequence 130 | cMhvSD095d06a2 |

AGGTACATTTTGAACTCCCAATTCCCACCCACAGAGCTTGGTGCTAGCTCTGCACACGGTAGATAT
AAGCAAGAACTTAGGCCGAAGTGAATTGAATGACCCATTCTTACCAGATAATTCTGTTCTTGCAGG
GGTATTTCGGATCTGGGTTCTGCCTCAAGGCTGACGGAATCAATACATTCAGCAAGTGTATCCTCA
GTCACGTCTCCATTGAGAGGGGGCTCCAGGGCGTTGGCATCCTGAGGCTGCACAGGGGGCCCAAT
GGCGGCAGCCCCTGCACCCTGCACAGCTGCATTTTCATGCCCCTCCCCTCTGGGGTCAGCTGGTGT
TGGCTCATGTGAAACTGCAGCTGAATCACAATGCACTTCTGGCATCCTCAGGTAAAGAATCACTAT
TAGGCATCTCAGTAACTTCTGCTTTGTCTCCAGTGGCTAAGGTGTCACCCAGCATCATCAGAACAT
TTTTAGTATCGCTCAAGGCGGCCCGCTCTAGNAACTAGTGGGATCCCCCCGGGCTGCAAGGAATTC
CGATATCAAAGCTTATTCGATACCCGTCAACCCTCNAAGGGGGGGGCCCCGGTACCCCAACTTTTT
TGTT

| | |
|---|---|
| Sequence 131 | cMhvSD095f05a2 |

TGCTTCTGCTATGGCGAGGAGTCCTCGGCCTCCAGCCACTGTGCCCACGCCTACCGGTTTTCTGGG
GATGTTGCCACCACCTCTGAAGAGTGAAACCAAGCTTTCCATGCAGGAAGAGCCAGGTGCTGGGG
GCTCCCGCCCGAACTGTGAGGCCCACAGTGCTTAGGGAGAGCACCAGGCTCTACCTTTCTTTCTTG
ACAGTGGGTGAGCAGCGCAGGCAGAGATGTGCAAGGTACCT

| | |
|---|---|
| Sequence 132 | cMhvSD001a06a1 |

GGACCGAGGGTTTGGTGCACCTCGATTTGGAGGAAGTAGGGCAGGGCCCTTATCTGGAAAGAAGT
TTGGAAACCCTGGGGAGAAATTAGTTAAAAAGAAGTGGAATCTTGATGAGCTGCCTAAATTTGAG
AAGAATTTTTATCAAGAGCACCCTGATTTGGCTAGGCGCACAGCACAAGAGGTGGAAACATACAG
AAGAAGCAAGGAAATTACAGTTAGAGGTCACAACTGCCCGAAGCCAGTTCTAAACAATTATTTTT
ACTAAAATGCATAATTATGTGATAGTTATACATATACCAACCTGTTATGTGAGACAAGCTGACCTG
CAAGTAGTCCAAGGCCAGTGAATCA

| | |
|---|---|
| Sequence 133 | cMhvSD001b09a1 |

AGGTACACGTCTCTGTCTGGGCCTCGGCCAGGGTGCCGAGGGCCAGCATGGACACCAGGGCCAGG
GCGCAGATCACCTTGTTCTCCATGGTGGCCATTGCCTCCTCTCTGCTCCAAAGGCGACCCCGAGTC
AGGGATCCCCGCGTACCTGCCCGGGCGGCCGAGGTACCAGCCGCTCATGTTTTTATCGCACCCCTG
GGACCCTGCTGAGTTCTCTGTGCTTCGGAAGGGTTCATCCAGGAGGGTGTAATTCTGACAGGGGTC
AAAACAGACATGAGCCTCTGGGGTGCCAGGAGCTCCGCAGTCCAGGTCCAGCCCATACGAACTGG
CTTCAATGGGGTTTCCATAACCTCG

| | |
|---|---|
| Sequence 134 | cMhvSD002a08a1 |

AGGTACTTGAGCCTAGGCAACAGAGCCAGACTCAGTCCTTTAAAGAAAAAAAAAAATTCTCCCAA
CTTCATAAGTAAACTGCCTAAACAAATCAGGATTCATTTTACCATTCATTTAGCAGAAGAGGAAGG
TAACAGAAGTTCATATATTTCGCCAGATAACTTTATCACCCTCCAACCCAGACTAGAGGTTTTGAT
TTAATTATCTCAAATGAACTTTAATTATTTTGAACTTATGATTACCATAATACCTCTTGTTAGAAAA
GTGAGATTTCTAAAACCTAGTAAGTAATCGTAAAGGTATAATTTTACCACCAGTAATGCAAGTTCT
TAACAGCTGTCTTGGCCTCAGGGGTCATAAACTAATGGCCTCAGTAATAAAATATTTAATAGAAAT
TAATGAGATAGGCCCAATGATGTGGGCCAAGTAAAGAGAGGAGAAATAAGAATTGGTGGGAACT
GTGGCAAATCGGAGAGAGTATGCACATCTAAAGGGACTCAGAGCAGGTTAATTCCAGCCCCTGTA
TACCCCGCGTACCTGCCCN

| | |
|---|---|
| Sequence 135 | cMhvSD002c03a1 |

CCGGGCAGGTACAAGGGGCATTGTCAGTGAGTGGTAATACTTTGAAAGGAATCTTATTTCTTGAGC
AGTAGTTGTCGACAGTGGGCTTAAGATATTCAATAAACCATATTTGTAAACCGGAAAAAAAAAAA
AAAAAAAAAAAAAGTNCCT

| | |
|---|---|
| Sequence 136 | cMhvSD003c02a1 |

GGCAATTGGAGCTCACCGCGGTGGCGGCCGCCCGGGCAGGTACGCGGGGGGTCCCAGCGTCGCTC
CGGACGCTGCCAACCTGTTCTCCACCGTCGCTCGACTTCCACCTCTAAGACTCCCACCTTCAAGATC
CTTCTGTCTAGTGTTTTGGGTTCCCTACACCAGGATTGTGGAGGAAGCGCACGGCCAGAACCCGTT
GGGACCGAGCAGATCAACCATTTATGTTGCACTTAATGATCATCTGCACTTTTTGCATATCCTTAGT
GTTGTCTTTGTGAGGCCACCTCTATAATGGATAATCAAATAGAGGGAAGGGCGGGATTGAATATTG
TGACTTGATTTCAATGTCCCACAACAACTGTGCTAGACAGTTTTTATATGTTAGGTTATTTAACGCT
CCCAAGCACTTATTAAAGTGATGTTACTCTGTTTCATTCTCCAGGAAACTCAGGTTGAATAATTCAT
CAAATTACACAACTGAACTCAAAGACATGGCTGCCCAGTGTGTCACAAAGGTGGTGCTGAATGTTT
CCCGTGCCAATCTTT

| | |
|---|---|
| Sequence 137 | cMhvSD003c02a1 |

GGCAATTGGAGCTCACCGCGGTGGCGGCCGCCCGGGCAGGTACGCGGGGGGTCCCAGCGTCGCTC
CGGACGCTGCCAACCTGTTCTCCACCGTCGCTCGACTTCCACCTCTAAGACTCCCACCTTCAAGATC
CTTCTGTCTAGTGTTTTGGGTTCCCTACACCAGGATTGTGGAGGAAGCGCACGGCCAGAACCCGTT
GGGACCGAGCAGATCAACCATTTATGTTGCACTTAATGATCATCTGCACTTTTTGCATATCCTTAGT
GTTGTCTTTGTGAGGCCACCTCTATAATGGATAATCAAATAGAGGGAAGGGCGGGATTGAATATTG
TGACTTGATTTCAATGTCCCACAACAACTGTGCTAGACAGTTTTTATATGTTAGGTTATTTAACGCT
CCCAAGCACTTATTAAAGTGATGTTACTCTGTTTCATTCTCCAGGAAACTCAGGTTGAATAATTCAT
CAAATTACACAACTGAACTCAAAGACATGGCTGCCCAGTGTGTCACAAAGGTGGTGCTGAATGTTT
CCCGTGCCAATCTTT

| | |
|---|---|
| Sequence 138 | cMhvSD003f08a1 |

CCGCGGTGGCGGCCGAGGTACTGGGAATGGGAAGTTTTCTGAATAAGGGTAACATGGGGCAGAAT
TTGTCTATTGAGGTGCAACATTATGTGCATTTGCTTAAAGTTTTACTTAAACAAACTGGTGCTCAGG
TTAGTTCTCAAACATTAATTAAGATGCTGAAGAAGGTCACTATACATAACCCGTGGTTTCCACAGA
CAGGCAGTCTTGATGTAGAAATTTGGGACAGAGTAGGACCAGGATTAAAACGGGCTCACCAAAAA
GGTCTTAAATTTGATCTTTTTGTTTTTTCTGCTTGGAGTTTAGTCCGTGCTGTCCTCCTGCCATTATC
TTCTTCTTATTCTGCTAGACAGCAGGAATCATATTCCGAGTCTAAAAATCTGAAAAAATATTTTGTC
CCACCCACAGTACCTGCCCG

| | |
|---|---|
| Sequence 139 | cMhvSD004d09a1 |

CCGGGCAGGTACGCGGGGTAACTTTTTAACTTTATAAACTTAGTATTTTAACTTTTTAAACTTTTTT
GTTGAAAACTAAGACACAAAAACACATGTTAGCCTAGATCCACACAGGGTCAGGGTCATCAGTAT
CACTGTCTTCCACCTCCACATTTTGTCTCTGGAAGGTCTTCAGGGGCAATAACACACATGGAGCTG
TCATCGCCTGTGGTAACAACGCAGAGTACCT

| | |
|---|---|
| Sequence 140 | cMhvSD004f03a1 |

CTACTATAGGGGCGAATTGGAGCTCCACCCGCGGTGGCGGCCCGCCACAGTCGCTGCGGAGGGGT
CTGAGGACAGGCGGTCCTGACTCCCGCTGCCCGGTGGAACTAAGACCAGGGACGAGGCCACGCAG
GAGATCAAGGTACCTNTNN

| | |
|---|---|
| Sequence 141 | cMhvSD004h08a1 |

CCGCGGTGGCGGCCGCCCGGGCAGGTACAAGCAGTAATTGATTCACTGGCCTTGGACTACTTGCA
GGTCAGCTTGTCTCACATAACAGGTTGGTATATGTATAACTATCACATAATTATGCATTTTAGTAA
AAATAATTGTTTAGAACTGGCTTCGGGCAGTTGTGACCTCTAACTGTAATTTCCTTGCTTCTTCTGT
ATGTTTCCACCTCTTGTGCTGTGCGCCTAGCCAAATCAGGGTGCTCTTGATAAAAATTCTTCTCAAA
TTTAGGCAGCTCATCAAGATTCCACTTCTTTTTAACTAATTTCTCCCCAGGGTTTCCAAACTTCTTTC
CAGATAAGGGCCCTGCCCTACTTCCTCCAAATCGAGGTGCACCAAACCCTCGGTCC

| | |
|---|---|
| Sequence 142 | cMhvSD005b02a1 |

CGTAATACGACTACTATAGGGGCGAATTGGAGCTCACCGCGGTGGCGGCCCGAGGTACCTGTTGG
CTTCATTTCTCTTATTACCCTGTTGCCAGGCCACCGGGTCCGGCCCAGCCTTGATTCTTCGGGAATC
ACTTCTCCCTCGCCGCGCCTGTTACTGCCTCCACGGATCACTCATCCTCGCTTCGCGTTCTTCCACT
AAAGAACCTGGGGCGCCGCACTACAGCGCCGCGGCCTCCCCGCGTACCTGCCCG

| | |
|---|---|
| Sequence 143 | cMhvSD005c07a1 |

CGAGGTACTAGCAGTAATTGATTCACTGGCCTTGGACTACTTGCAGGTCAGCTTGTCTCACATAAC
AGGTTGGTATATGTATAACTATCACATAATTATGCATTTTAGTAAAAATAATTGTTTAGAACTGGC
TTCGGGCAGTTGTGACCTCTAACTGTAATTTCCTTGCTTCTTCTGTATGTTTCCACCTCTTGTGCTGT
GCGCCTAGCCAAATCAGGGTGCTCTTGATAAAAATTCTTCTCAAATTTAGGCAGCTCATCAAGATT
CCACTTCTTTTTAACTAATTTCTCCCCAGGGTTTCCAAACTTCTTTCCAGATAAGGGCCCTGCCCTA
CTTCCTCCAAATCGAGGTGCACCAAACCCTCGGTCC

| | |
|---|---|
| Sequence 144 | cMhvSD005h11a1 |

AGGTACTTGNNCCAAATGTGCAACATNAATNCGGAACCNANGANCANAAGACTNNTTACCNATAC
TGGAACNNGGNCAANTNNNANCCCACGNGAATNTTCTNNGTCANATNNCCACATCCNCNCNGTGC
TGCNGAGGNTGTGCNGACTGNACTNCTTGTNCNANANNNGNCNTTNNNNCTCTNCCNNACNGNNN
ATNCCNNTGCCC

| | |
|---|---|
| Sequence 145 | cMhvSD005h11a1 |

NGAACATCAACTTTTGANCTTTTAGTGANGGTATATANCGCNCTCGGNCTTNNNATNGANATNCCT
TGTNANTGTGNNAAATCTGTATCNCGCTTACAATAACTACCNACGTANGCAGCCGNGAGCATANG
AGC

| | |
|---|---|
| Sequence 146 | cMhvSD005h12a1 |

NCGCCCGGGCAGGTACAGGTATTTGTTGCATTATTCTAACAACTTTACTGCAGATTTCACTTTTTCA
AAACTAAAAGTTGAGGGAAGGGGAAACACCAAAAAACCCTCCCACGGCCACTCGCCCTGCTTGGG
CTGCTGCTTTTTGAGATCTCANAAAGTTGGACAAGGGCCATGACCAGCAGCCTGNTCCAAAACAA
CAACTAGGAACCTGCTGTGGGTCACAAGCTTGGGAAGCTGCTGGGGGCAGATTTCACTTTGTGCTT
CTGGGTGAGGGCAGGGGCGTGAGGGTGATAAAATACTTTTGTGAGCTGAACAGNGGGGAAACAA
AAGTTTCAAAA

| | |
|---|---|
| Sequence 147 | cMhvSD006e04a1 |

CCGCGGTGGCGGCCGAGGTACCTTCTCACACCTGCGTTCTTTTCTTGAGAGATACTGTGATAAAAT
AAACAGTGAGATTCCCCCACTCCCTTTCCCTTCATCAAGAGAACACCACAGTTTTCTCAAGCTGTG
CCTGAAGCTCTTTCAAATCACCTTGCTCTTGCACTTGCGGGAGGGGTAGCTACCAGCATTCTCGGG
AGGCAGGCAGGTCCACTTCGAAATTTGCTCTTCAGACTGATGGACTCAACTGTCCCAGATGAAATC
CAAGAGTAATGAAGATATTCTAAATTGGATAGTGGTGATGGTTGCACAACTCTGAATAGACTAAA
AACCATTGAATTTTATACTTTCAAGAGGTGAATTCTGTGGCATGTGGATTATATGTCAATTTGAAA
AAAAAAAATAAACTGACTTTTCAAGTAGAGGGACATATCCCCTCAAATGGGGTTGGAGGAATATC
CTGGTGGTGAGTAGGAACTGTGATGATTTAATATTTATCAGAAACGGGGTAGTGTAAGATTTTGAA
AAGGGTNAAAAGTACCTGCCCGGCCGGCCGCTCTAGAACTA

| | |
|---|---|
| Sequence 148 | cMhvSD007g03a1 |

CACTACTTAGGGCGAATTGGAGCTCCCCGCGGTGGCGGCCGAGGTACACTCTTCCTTAAGTCCAGT
GGTGCAGGAAAGCTTCAGTTTGTCAATATCACGCAAGACAGGGACACCAAACACTACCCCTGCCC
AAAGGAGCCCCTCACGGACGCCGCCATGTTGTTACCGGACCCCCCCGCGTACCTGCCCG

| | |
|---|---|
| Sequence 149 | cMhvSD007g04a1 |

ACTTAGGGCGAATTGGAGCTCACCGCGGTGGCGGCCGAGGTACGCGGGGGAGGAACTGCTCAGTT
AGGACCCAGACGGAACCATGGAAGCCCCAGCGCAGCTTCTCTTCCTCCTGCTACTCTGGCTCCCAG
ACACCACTGGAGAAATGGTGATGACG

| | |
|---|---|
| Sequence 150 | cMhvSD007g04a1 |

GTCACGATATTACTACCCACTTAGCCTGGTACCTGCCCGGGCGGCCGCTCTAGAACTAGT

| | |
|---|---|
| Sequence 151 | cMhvSD007g04a1 |

TAGTGAGGGTTAATTTGCGCGCTTGGCCGTAATCATGGTCATAAG

| | |
|---|---|
| Sequence 152 | cMhvSD008d08a1 |

ACTTAGGGCGAATTGGAGCTCCCCGCGGTGGCGGCCGCCCGGGCAGGTACGCGGGATTCCTGGCT
TTTTAACTTTNNCAAATGTAACCTCCCATGTGCTNNGAGAAAGGAAAATTTAAGACAGCTTATGAA
AGGGAGGAGAANCAACANATGGNNCAGGTCACCCAAATGCCAACCATGAAAGNGCTCATTTTCTA
GGCTAAAAATTGAACCTGAACTCAGGCCACCATNGTGAAAAGACAAAGCCTTAACTGCTAAGCTA
CACGCATTGGGCAGTTTCCACTGCTTTTCCCAGAAGGAGCCCANAGCAGGGAATTTTGAGCTTGCA
AAGGCTTTTAACTGCTCAAGATAATTNGNANAGCTAACTACTACCCCAAAATCCC

| | |
|---|---|
| Sequence 153 | cMhvSD008e08a1 |

CTACTTAGGGCGAATTGGAGCTCCCCGCGGTGGCGGCCGAGGTACGCGGGAAGATCTACACTATT
ATGTCACCCCAGAAAGTGAACTCTCAGTCTTCCCAGCCAGTCTCTTTCTTATCATAGGTTAGCTTGC
TTATTCTGGAATTTCGCGTATACAGATGCATGCCATGCCATAGGTACCTGCCCG

| | |
|---|---|
| Sequence 154 | cMhvSD008f08a1 |

GGGCTATTGGTTGAATGAGTANGGCTGATGGTTTCGATAATAACTAGTATGGGGATAAGGGGTGT
AGGTGTGCCTTNTGCTAAGAACTGNGCTAGGNCNTTTNCAANNTTACNNCNAAAGCCTATAATCA
CTGCGCCCCCCGCGTACCTCN

| | |
|---|---|
| Sequence 155 | cMhvSD008f08a1 |

CGGGCTGCAAGGAATTCGAATNTCAAGCTTTATCGATACCCGTCCNACCTTNTATNGTNGTGGGCC
CGGGAAACCCCAAATTTTTNGCTTCCCCTTTTANATGAAGGGGTTAAATATGCCGCCGCCTTGGGC
CGTTA

| | |
|---|---|
| Sequence 156 | cMhvSD008g09a1 |

CCGCGGTGGCGGCCGAGGTACAAGCAGTAATTGATTCACTGGCCTTGGACTACTTGCAGGTCANCT
TGTCTCACATAACAGGTTGGTATATGTATAACTATCACATAATTATGCATTTTAGTAAAAATAATT
GTTTAGAACTGGCTTCGGGCAGTTGTGACCTCTAACTGTAATTTCCTTGCTTCTTCTGTATGTTTCC
ACCTCTTGTGCTGTGCGCCTANNCAAATCAGNGGTGCTCTTGATAAAAATTCTTCTCAAATTTAGG
CAGCTCATCAAGATTCCACTTCTTTTTAACTAATTTCTCCCCAGGGTTTCCAAACTTCTTTCCAGAT
AAGGGCCCTGCCCTACTTCCTCCAAATCGAGGNGCACCAAANCCTCGGTCCCN

| | |
|---|---|
| Sequence 157 | cMhvSD009c12a1 |

TGGAACNCCACCGCGGTGGCGGCCGCCCGGGCAGGTACCTTTTTGCCCTGCAGGGACTGNACCTG
CTGTGGGATTTGAATACAAATGGTGGAACACGCTGCCCACAAACATGGAAACGACCGTTCTCAGT
GGGATCAACTTCGAGTACCT

| | |
|---|---|
| Sequence 158 | cMhvSD009f06a1 |

CCGGGCAGGTACCCAGGGAACAAATGCTACTGGGACTCCACACCTACCTAAGAAGCAGCTCTACC
CAGACTCCACATGGCTCTCTGTTTTGGTCTGGAGACCCCAGCTGGGGTATCTCCTGAGCCCAGGGA
TTCAAAGGTTCGTGGCAGAAATATGCATCCCACGGGACTCTCACTCACTCACCATTTTCTTGTAGG
GGGATTCCCCTGGGTCTGTGCCACTCCTGGGTGAATGGCTGATCTGTCTCACTCTTCTCCGTGATCC
GAAGGTCACACTATGTCACTGATGAATCCTTATGTGTCCACCTGGATGTTCCGGTTGAAGAGCTAG
TGTCTCACCACTCTT

| | |
|---|---|
| Sequence 159 | cMhvSD009g03a1 |

CGCCCGGGCAGGTACTCTCCCTCTTTTCCTAGGGATGTGGCTTCCTGAGAGCCAAGTTGTAGTGAC
TGTCATCTCTCTTGTGGATCTAGCCACCCAGCAGGTCTACCAGGCTCTGGGCTGGTGCTGGGGGTT
GTCTACACTGGGTCCTGTGATGTGAACCATCTGCAGATTTCTCAGCTATGGGTACCT

| | |
|---|---|
| Sequence 160 | cMhvSD010b09a1 |

CCGGGCAGGTACCTGCCACATGTCGGGCCGGTCAGCACAGGTTTTCTGCAGGGCTTCTGGCTGGGC
TGGCAAAAAGCAGCAGGGAGCAGGACAAAGCTTTTTTTCTGGCCTGACTCCCCCTTGCTGAGCCCA
GCGCTGCCACCTGGGTGGATGGTCCCCGGGGCCCTATTCCCAGTTGCTCCAGAGCCACTATTTAGG
ATCCAGGTTGTGCCACCAAGTTCAAGGCTGGTTGTGATGGTGAGAACAGCTGCTTTCATAGAAAAA
TCATCATGTCCTAGCACAGATGGCCCCAAGCAGGGGAAGTACCT

| | |
|---|---|
| Sequence 161 | cMhvSD010c04a1 |

CCGGGCAGGTACCAAGCAGAAACCTGGCCAGGCTCCCAGGCTCCTCATCTATGGTGCATCCACCA
GGGCCACTGGTA

| | |
|---|---|
| Sequence 162 | cMhvSD010c04a1 |

CGCAGTATAATAACTGGCCTCCGACCACCTTCGGCCAAGGGACACGACTGGAGATTAAACGAACT
GTGGCTGCACCATCTGTCTTCATCTTCCCGCCATCTGATGAGCAAGTTGAAATCTGGAACTGCTCTG
TTGGGTGCCTGCTGAATAACTTCTATCCCAGAAAAGGCCAAAGTACCTNGGGCCGCTCTAGAACTA
GTG

| | |
|---|---|
| Sequence 163 | cMhvSD010c09a1 |

AGGTACAAGTCATAATCTCTTTTCAAGCCGGCCTAGCCCCTTCCCGGAACCTCGGCTCCCCCCCAA
CGAAACTACTGCTAAGCCAACTGGACTACACTTCCCAGACTGCTTGGAGCCTCTCTCTCCGCAGAA
CCTCGTCTTCCGCGAGCTTTTCCTGGAGGTTCTAGGAGGGATGCCCCTCAATGCCACGACGCCATT
TCCTACTACCCCCGCGTACCTGCCCGGCGGCCGCCCGGGCAGGTACAGCAAAACCCACCTGTGTAA
ACACACACAGCAAAGTGATGTAAGAAGTTTCCATATAAAGGGCTGCAGTATGGAGAGGTAATGTG
CAGGCTGGTTTGCGGCTGTAGGGGCCACCTTGCTGCAGCTCTCCACTGATATGGTACCTCGGC

| | |
|---|---|
| Sequence 164 | cMhvSD010d08a1 |

CCGCGGTGGCGGCCGCCCGGGCAGGTACCGCAGCAGAGCACTCTCAGCTCTGGGTCTTGCAGGCG
CAGGGCTCCCCCATGCCAGCAGAAAGATTTCCTCTGGACAGGCGACACTAACAGGTGAAGATCTC
GGGAGACCATGACTAAGAAAAGAATTGCTGTGATTGGGGGAGGAGTGAGCGGGCTCTCTTCCATC
AAGTGCTGCGTAGAAGAAGGCTTGGAACCTGTCTGCTTTGAAAGGACTGATGACATCGGAGGGCT
CTGGAGGTTCCAGGAAAATCCTGAA

| | |
|---|---|
| Sequence 165 | cMhvSD010f12a1 |

CCGCGGTGGCGGCCCGAGGTACAAGCAGTAATTGATTCACTGGCCTTGGACTACTTGCAGGTCAGC
TTGTCTCACATAACAGGTTGGTATATGTATAACTATCACATAATTATGCATTTTAGTAAAAATAATT
GTTTAGAACTGGCTTCGGGCAGTTGTGACCTCTAACTGTAATTTCCTTGCTTCTTCTGTATGTTTCC
ACCTCTTGTGCTGTGCGCCTAGTCAAATCAGGGTGCTCTTGATAAAAATTCTTCTCAAATTTAGGCA
GCTCATCAAGATTCCACTTCTTTTTAACTAATTTCTCCCCAGGGTTTCCAAACTTCTTTCCAGATAA
GGGCCCTGCCCTACTTCCTCCAAATCGAGGTGCACCAAACCCTCGGTCC

| | |
|---|---|
| Sequence 166 | cMhvSD010g02a1 |

TCCTATAGGGCGAATTGGAGCTCCCCGCGGTGGCGGCCCGAGGTACTCAAAGGTGATATTTGCTTT
TTTCAATGCTTCAGGGGAAAAATCCTTTTCTTTACAAACTTCCATCAGTTTAGGAGTCAGTCTGTAT
GCCTTTAGTGAGAGAGATCCTTGGGCAGTTTTTATGGGATCATAAATGAGAACGACAGATTCTTCA
ATGGCATGCTGGTAACTAAACTGAGAGTCCGGGAGTGCCCGGGTAACGAATGAGCCATAGTATGT
GGACTGATACCAGCCCACGTGAAGATGATCAATGTTTACATGGCGAAGCTCCGCATCATTTCCATC
TTGATATTGGACAGAACCTCTAGCTGAGCTTGTCCTCTTCACACTGAGTAATGGGTTATGTTTCTTC
CCTGAGGGCCTAAACTTTTNATTTGNTCTTATTAAATATTATTCTCTTTTAAAAGCTTCTAAATTTC
AACTGGCCCTGATTAC

| | |
|---|---|
| Sequence 167 | cMhvSD010h04a1 |

CGGCCGAGGTACAGTGCAGAGGACTGGAATGGATATAATGTCTGCAAAACAAAAACATGTCTAGT
GAGCCATCTACTAATCTCAACCACTGGTCTAACTCATGACAGTCTCAAAATGAATATTTAAGAAAA
AAGTAGTGGCATCTAAAAATATAGACGTTTTGCAACTGACTCAGGGAGAGCTCTTTCTTCAACTAC
TGAATATACTGGTTTTAAATGATGGAGTGAGACAAAGAGGCTCTTGCTGACGTGCTCTACTTTGAT
TTCTATCCTAAAATCTAACAGGTAATCAATGTGTTTGGCTACCTATAGGAGCATCCACCAACTGAT
ATCATTTTTTTTTTTTTTTTTGAGATAGAGTCTCATTCTGTCACCTAGGCTGGAGGGCAG

| | |
|---|---|
| Sequence 168 | cMhvSD011c10a1 |

CCGCGGTGGGCGGCCCGCCCGGGCAGGTACAAGCAGTAATTGATTCACTGGCCTTGGACTACTTGC
AGGTCAGCTTGTCTCACATAACAGGTTGGTATATGTATAACTATCACATAATTATGCATTTTAGTA
AAAATAATTGTTTAGAACTGGCTTCGGACAGTTGTGACCTCTAACTGTAATTTCCTTGCTTCTTCTG
TATGTTTCCACCTCTTGTGCTGTGCGCCTAGCCAAATCAGGGTGCTCTTGATAAAAATTCTTCTCAA
ATTTAGGCAGCTCATCAAGATTCCACTTCTTTTTAACTAATTTCTCCCCAGGGTTTNCAAAACTTCT
TTNCAGATAAGGGGCCCTGCCCTACTTCCTTCAAATCGAGGTGCACCAAACCCTCNGTCCCGGC

| | |
|---|---|
| Sequence 169 | cMhvSD011e09a1 |

CCGCGGTGGCGGCCCGAGGTACGCGGGTCCCCATGTGTGACGCCGGTGAGCAGTGTGCAGTGAGG
AAAGGGGCAAGGATCGGGAAGCTGTGTGACTGTCCCCGAGGAACCTCCTGCAATTCCTTCCTCCTG
AAGTGCTTATGAAGGGGCGTCCATTCTCCTCCATACATCCCCATCCCTCTACTTTCCCCAGAGGACC
ACACCTTCCTCCCTGGAGTTTGGCTTAAGCAACAGATAAAGTTTTTATTTTCCTCTGAAGGGAAAG
GGCTCTTTTCCTTGCTGTTTCAAAAATAAAAGAACACATTAGATGTTACTGTGTGAAGAATAATGC
CTTGTATGGTGTTGATACGTGTGTGAAGTATTCTTATTTATTTGTCTGACAAACTCTTGTGTACCTG
CCCCGGGCCGGCCCGTTCTAGAAACTAGTGGGATCCCCCCGGGCCTGCNANGAAATTCGATATCA
AGCTTATCCGATACCGTCGAACCTCGAGGGGGG

| | |
|---|---|
| Sequence 170 | cMhvSD011f10a1 |

CGCCCGGGCAGGGTACTTGGATTACAGGCGTGGACCAGCATGCCATGCCTATAGTGATATCTTTAA
GTAACCCTCTCTTTTCTTCTTTTGAGCAATTTTTCAAAGCAACAGGCATTTTATTAAATAAGAAAGT
CGATGTGCTTTCCTAATGCCTGTTAATAAAGTAAGGAGCCAAGGAACCTCTGTGATTTCAATGAAA
TCCCTCCAGATATTATAGGCTACTTGTTACTGACAAGTATGGCAGGAACTGCAGGTCAAGCTGTGA
TAGGCAAATAGATCTTGCTGAAGAGGAAGAATGATTGGCTAAGATAATGCCCCAAGACAGCTGGC
ATACCTTTAGACACAGCTAAATTGAATGCTTTCTGANGAGGAGTGTATTAAGTCTGTCTCACACTG
ATATAAAGACATACCTGAGAATGGGTNATTGAAAAAA

| | |
|---|---|
| Sequence 171 | cMhvSD012a08a1 |

GGTCTCGGTCACTCGAATAACCCGACATGGCGTCAATGGTTGCGGTTGGCGGGGAACGAAGTATA
TAGAAAAGCGTGCGACAAGTCGCTGGAAATGGCCTCGATGACGGCGAAGCCTTGCGGGGGCNGGC
AGCGGAGGAAGGACACCGATGACACCAGCCGAAGCTGCACTACTAGAGACCGGTAGAAATGAAT
GAGGTCCCCGCGTACCTCGGCCGCCCGGGCAGGTACAATGCAAAGTATAGGCTTTTGAACTAAATT
GGCCTGGGTTCAAATATGAGCCCTCTCACATTCTATTAGGTTGAACCATATAAAAATGGAGATATT
CAATCATTTTTTTACAGTTTCACGTAGTTCA

| | |
|---|---|
| Sequence 172 | cMhvSD012c04a1 |

CCGGGCAGGTACCTTTGGTTAAGAGTAGACAAGGCAGACATCTGAGCCTGCATGACTCAGCAAGT
TTAGGGTGCAGGCACATACTCCACTTGTTGTATAACCTGTTTGTGTAAGCTGATACTTGCCTTGGAG
CCACTATTGTCTGTAAAAGGTATAACTGCCCTGCTGACACTGTGCATGGGGGACATGGCTTGGCTT
GGCTCTTGGGCATGGCTTGACATGGCTCTTGCGCTCATGCCCAGAGAGAGAAGGAGATAAACTGC
TGACCCTGA

| | |
|---|---|
| Sequence 173 | cMhvSD012e09a1 |

CCGGGCAGGTACTTGGATNACAGGCGTGGACCAGCATGCCATGCCTATANTGATATNTTTAAGTA
ACCCTCTCTTTTCTTCTTTNGANCAATTTTTCAAAGCAACAGGCATTTTATTAAATAAGAAAGTCNA
TGTGCTTTCCTAATGCCTGTTAATAAAGTAAGGAGCCAAGGAACCTNTGTGATTTCAATGAAATCC
CTCCAGATATTATAGGCTACTTGTTACTNGACAAGTATGGCANGAACTGCANGTCAAGCTGTGATA
GGCAAATAGATCTTGCTGAAGAGGAAGAAT

| | |
|---|---|
| Sequence 174 | cMhvSD013d01a1 |

CCGCGGTGGCGGCCGCCCGGGCAGGTACAAGCAGTAATTGATTCACTGGCCTTGGACTACTTGCA
GGTCAGCTTGTCTCACATAACAGGTTGGTATATGTATAACTATCACATAATTATGCATTTTAGTAA
AAATAATTGTTTAGAACTGGCTTCGGGCAGTTGTGACCTCTAACTGTAATTTCCTTGCTTCTTCTGT
ATGTTTCCACCTCTTGTGCTGTGCGCCTAGCCAAATCAGGGTGCTCTTGATAAAAATTCTTCTCAAA
TTTAGGCAGCTCATCAAGATTCCACTTCTTTTTAACTAATTTCTCCCCAGGGTTTCCAAACTTCTTTC
CAGATAAGGGCCCTGCCCTACTTCCTCCAAATCGAGGTGCACCAAACCCTCGGTCC

| | |
|---|---|
| Sequence 175 | cMhvSD014d03a1 |

CGCCCGGCAGGTACGCGGGGAGAGGGAGCTGGGCAGGGCACAGCAGGGCAGGAGTGTGTTTGAT
GTGTCCTGGGAACCGCCCTGAGGCCGTCGTGTGGCTGGAGTGCTGCAGGTGTCAAGGAAATTGTA
GGAGATGTCTCCTGAGTGTGATGGAATATAACCAGATTTCCAGAAGGAACTGACATGATCTGACTT
AAAAAGGCCACCTACATTTACATGAAGGCCGCCTACCTCAGCATGTTTGGGAAGGAGGACCACAA
GCCGTTCGGGGACGACGAAGTGGAATTATTTCGAGCTGTGCCAGGCCTGAAGCTCAAGATTGCTG
GGAAATCTCTACCCACAGAGAAGTTTGCCATCCGGAAGTCCCGGCGCTACTTCTCCTCCAACCCTA
TCTCGCTGCCAGTGCCTGCTCTGGAAATGATGTACCT

| | |
|---|---|
| Sequence 176 | cMhvSD014f04a1 |

GGGGCCATTGAGACTGCCATGGAAGACTTGAAAGGTCACGTAGCTGAGACTTCTGGAGAGACCAT
TCAAGGCTTCTGGCTCTTGACAAAGATAGACCACTGGAACAATGAGAAGGAGAGAATTCTACTGG
TCACAGACAAGACTCTCTTGATCTGCAAATACGACTTCATCATGCTGAGTTGTGTGCAGCTGCAGC
GGATTCCTCTGAGCGCTGTCTATCGCATCTGCCTGGGCAAGTTCACCTTCCCTGGGATGTCCCTGGA
CAAGAGACAAGGAGAAGGCCTTAGGATCTACTGGGGGAGTCCGGAGGAGCAGTCTCTTCTGTCCC
GCTGGAACCCATGGTCCACTGAAGTTCCTTATGCTACTTTCACTGAGCATCCTATGAAATACACCA
GTGAGAAATTCCTTGAAATTTGCAAGTT

| | |
|---|---|
| Sequence 177 | cMhvSD015c06a1 |

GCGGCCCGAGGTACTGTCCAACTGGATGCTGCCCTGGTGGCTGAAGGCACACTTCATGATGCTGTC
CAGGGTCATCAGGGAGACATGTTGAAAGAGCTCCAGACGTGAGTTTTGGGCAATGTGTTCCTCCCA
TTTGTTCAGCATCATCCGAACACTCTCAGACATCATGGTGATGAATATTTTCAGAATGCTGATGTTG
AAGCCAGGTTTCACAATCTGGCGGTGCTTTTTCCATTTAGAACCATCCAGGGTCACAAGTCCTCGA
CCAACCCAGGATTCAAGGATTTTGTGGCTAACAGCACTTTTGGGATCTTGTCTTTTCGGGAGAATC
TTGGCATAGTCTGGGTCATGGACACTGAAGAACATCGTAAAGGGTCCAACCCACAAGGGAACAGC
ACATGGGTATTTTTCCATCAGCTTATGATACACCTCAAACTCCTTTACTGGGTAAAACTCCTTGTGG
CCATAAACCAAGTGGGCAGGGGGTGCANGAAAACAGGTGCAGGGCTCTGAACATCCATCTCCTCC
TNTGGTACCTGC

| | |
|---|---|
| Sequence 178 | cMhvSD016d08a1 |

AATNGGAGCTCCCCCGCGGTGGCGGCCCGGCCATGGAGGCTGATGGGGCCGGCNAGCACATGAGA
CCNCTACTCACCCGGGGTCCTGATGAAGAAGCTGTTGTGGATCTTGGCAAAACTAGCTACNNTGTG
NAACCNAAGTTNANACANANGAACTTGAAGAGTCATANAGCTGTNTATNNTGGAGTTCACGTCCC
GTTTAGTAAAGAGAGTCGTCGGCGTCATAGGCATCNGTGACACAAACATCACCACCAAAACGNAN
GNNANATANTTNAAANAAAAGTCCTCNGCCGCTCTAGAACTAN

| | |
|---|---|
| Sequence 179 | cMhvSD016f01a1 |

GGAGCTCCCCGCGGTGGCGGCCGACGTNCAAGNATCTGTTGCNTGCACATCTNCGATAGCCAACG
CCTGNCCATNATTGGNCNNATANAAACCCTCNTGCTNCATGATACCTACAGGANAAACACAANCT
CGGTNNGCTNTTCGAGTNCTGAAAGGTGTGAATAAGTTACCACCACCAAGTGTCATGATAGAGGA
AATTAATGCAAGGAAAGAAAACAAGCCCAGTTGTTCCGCTTGACTGGCCCAGGAAAATGGGAAGG
AGCCAGAAATGCCATCATGACCCAGTGGGACCGAACATTCAAGGTCATCAAAGCTCGAGTTGTAC
CTGCCCG

| | |
|---|---|
| Sequence 180 | cMhvSD016f07a1 |

CCGCGGTGGCGGCCCGAGGTACCCAGGGAACAAATGCTACTGGGACTCCACACCTACCTAAGAAG
CAGCTCTACCCAGACTCCACATGGCTCTCTGTTTTGGTCTGGAGACCCCAGCTGGGGTATCTCCTG
AGCCCAGGGATTCAAAGGTTCGTGGCAGAAATATGCATCCCACGGGACTCTCACTCACTCACCATT
TTCTTGTAGGGGGATTCCCCTGGGTCTGTGCCACTCCTGGGTGAATGGTTGATCTGTCTCACTCTTC
TCCGTGATCCGAAGGTCACACTATGTCACTGATGAATCCTTATGTGTCCACCTGGATGTTCCGGTTG
AAGAGCTAGTGTCTCACCACTCTTTCTGCTATTTGTGAGAAGTGGCACACACTAGCTGCTTCTAGTC
AACCATCTTGGCCCCACCTCACTCACTTTTCTCAAGTAATCAAAGACCAGAAAGGATGTCCTTTAC
AAGAAGCAGATCCCCCAAAATGTAAGAANTCACTTGAAAANGNGGGGAGCTCAAACCCAAGANA
AGGACTTATCTNGCAGCATAAAAAACAACTTGTACCTGCCCGGGCCGGGCCGNTTTAGAACTANA
GGGATCCCCCGGGCTGANGGAATTNATTTNANCTTATTGATNCCNNNGACCTNAGGGGGGGGCCC
GGTN

| | |
|---|---|
| Sequence 181 | cMhvSD018b02a1 |

AGGTACTTTTTTTTTTTTTTTTTTTTTTCCTTTTTTTTTTTTTTTTTTTTTTTTTTTTTTTTTTTTTTTTTT
TTTTTGGGNCCCNCCANNCTTTGATTGGCCCNCAACANTNTTACAAACAAAAGGCATTAGGCAAA
GCATGCNNAATTGATNGGAGNCCCTTGGNCAAAGGTNTTATTGATTGACGGCAATCAAANCCNCN
CCCTNAAAAAGGATTTGANNAGGCCNNTTNTGNCCATNTGCAAAAGGNTCCCCAAAAGGGGCAAA
NGGCGGGGCCCNGGNGGNAGGGNNCCATGGGANTTAGGGNGACCCNNAACCANNANTACCAANA
GGCCTNTNAGGANTGCAANGAAAAANAGGACCCTNANCNCCATGGTTCCAGNNTNACTGCCCTGC
CCCCGNGTACCTGCCCG

| | |
|---|---|
| Sequence 182 | cMhvSD018b02a1 |

CCCCCTGGNGAAANANGGGCANAACNGNTNCCNGGGGAAAANNNTNTCCNNTAAAATNCNCAAA
ATANAAACCNGGAACAAAANNGAAAACCC

| | |
|---|---|
| Sequence 183 | cMhvSD018h06a1 |

AGGTACAAACTTAGAAGAAAATTGGAAGATAGAAACAAGATAGAAAATGAAAATATTGTCAAGA
GTTTCAGATAGAAAATGAAAAACAAGCTAAGACAAGTATTGGAGAAGTATAGAAGATAGAAAAA
TATAAAGCCAAAAATTGGATAAAATAGCACTGAAAAAATGAGGAAATTATTGGTTACCAATAGAA
GGGCAATGCTTTTAGATTAAAATGAAGGTGACTTAAACAGCTTAAAGTTTAGTTTAAAAGTTGTAG
GTGATTAAAATAATTTGAAGGCGATCTTTTAAAAAGAGATTAAACCGAAGGTGATTAAAAGACCT
TGAAATCCATGACGCAGGGAGAATTGCGTCATTTAAAGCCTAGTTAACGCATTTACTAAACGCAG
ACGAAAATGGAAAGATTAATTGGGAGTGGTAGGATGAAACAATTTGGAGAAGATAGAAGTTTGA
AGTGGAAAACTGGAAGACAGAAGTACCTCGGC

| | |
|---|---|
| Sequence 184 | cMhvSD019b10a1 |

AGGTACAAGTTGTCTTTATGCTGCGAGATAAGTCCTCTCTTGGTTTGAGCTCCCACCTTTTCAGTGA
ACTCTTACATTTTGGGGGATCTGCTCTTGTAAAGGACATCCTTTCTGGTCTTTGATTACTTGAGAAA
AGTGAGTGAGGTGGGGCCAAGATGGTTGACTAGAAGCAGCTAGTGTGTGCCACTCTCACAAATAG
CAGAAAGAGTGGTGAGACACTAGCTCTTCAACCGGAACATCCAGGTGGACACATAAGGATTCATC
AGTGACATAGTGTGACCTTCGGATCACGGAGAAGAGTGAGACAGATCAGCCATTCACCCAGGAGT
GGCACAGACCCGGGGGAATCCCCCTACAAGAAAATGGTGAGTGAGTGAGAGTCCCGTGGGATGCA
TATTTCTGCCACGAACCTTTGAATCCCTGGGCTCANGAGATACCCCAGCTGGGGTCTCCAGACCAA
AACAGAGAGCCATGTGGAGTCTGGGTAGAGCTGCTTCTTANGTAGGTGTGGAGTCCCAGTAGCAT
TTGTTCCCTGGGNACCTGCCCG

| | |
|---|---|
| Sequence 185 | cMhvSD019b10a1 |

NNANATCAAGCTTATCNATCCCGCNACCTCNAGGGGGGGCCC

| | |
|---|---|
| Sequence 186 | cMhvSD019c04a1 |

AGGTACGCGGGAGATTATGAAAATCGCGAGTCAACACCCAAACTGGCAAAATTACTGAAACTACT
ACTTTGGGCTCAGAACGAGCTGGACCAGAAGAAAGTAAAATATCCCAAAATGACAGACCTCAGCA
AGGGTGTGATTGAGGAGCCCAAGTAGCGCCTGCGCTTGCGTGGTGGATCCAACACCAACCCTGCG
TCGTGGGACTTGCCTCAGATCAGCCTGCGACTGCAAGATTCTTACTGCAGTAGAGAACTCTTTTTCT
CCCTTGTACGCGGGACCTGGACGAAGGCTTGTCCTACACGAGCATCTTCTATCCGGTTGAAGTTTT
TGAGAGTTCGCTTTCAGATCCTGGGCCCGGAAAGCAAGA

| | |
|---|---|
| Sequence 187 | cMhvSD019f07a1 |

GGTCTCGGTCACTCGAATAACCCGACATGGCGTCAATGGTTGCGGTTGGCGGGGAACGAAGTATA
TAGAAAAGCGTGCGACAAGTCGCTGGAAATGGCCTCGATGACGGCGAAGCCTTGCGGGGGCGGCA
GCGGAGGAAGGACACCGATGACACCAGCCGAAGCTGCACTACTAGAGACCGGTAGAAATGAATG
AGGTCCCCCGCGTACCTCGGCCGCCCGGGCAGGTACGCGGGGGCCAGCGTCACCAGACCAGCTGC
GGGACAAACCACTCAGACTGCTTGTAGGACAAATACTTCTGACATTTTCGTTTAAGCA

| | |
|---|---|
| Sequence 188 | cMhvSD019f08a1 |

AGGTACTAGCAGTAATTGATTCACTGGCCTTGGACTACTTGCAGGTCAGCTTGTCTCACATAACAG
GTTGGTATATGTATAACTATCACATAATTATGCATTTTAGTAAAAATAATTGTTTAGAACTGGCTTC
GGGCAGTTGTGACCTCTAACTGTAATTTCCTTGCTTCTTCTGTATGTTTCCACCTCTTGTGCTGTGCG
CCTAGCCAAATCAGGGTGCTCTTGATAAAAATTCTTCTCAAATTTAGGCAGCTCATCAAGATTCCA
CTTCTTTTTAACTAAATTTCTCCCCAGGGTTT

| | |
|---|---|
| Sequence 189 | cMhvSD021a11a1 |

CCGGGCAGGTACATTTCCTGAGCAGGTGATCCTGGCTGTCTGTCCTGGAGACACTGACACTGAAGA
TGGCTGTGTCAGCTCATAGGAGGCCACAGAGACTGTGCAGAGAATGAGGAGGGGGAGCAGGAGA
GGGATCCAGGCCATGGTGAGACATTCAGAGCTCTGCCTCCTGAGCCTACAGCCCCCGCGTACCTCG
GCCGCCCGGGCAGGTACTTTAATAGCTCAAACTCAGAGTCATCGTGCTCCCAATTCCAAAGAGATT
CCTAAAAGAGGCAACTT

| | |
|---|---|
| Sequence 190 | cMhvSD022b06a1 |

CCGGGCAGGTACCTTCTGGGGCATACAACATGGCAGCAGGGCCTCGGGAAGAGGGGTAGGAGGA
CCGAGCAGCATTCTCTGTAGAGGAAGACAGGAAAGGAGACCCTCTTGGCACACATTTATGGAGGG
TTGTCCCTGAAGAGAAGGGCAGGTGGGAGAGGTTCCCTGTTACTTAAGAGAAGGCACCAGTGGCA
AAGAGCACAATGAAGAGGATGATGATAAAAACAATCACGCAGATAAGGACAATCATCTTCACGTT
CTTCCACCAGAATTTTCGAGCCACCTTCTGCGATGTCGTCTTGAAGTGCTCAGATGTGGCTTCCAGA
TCCTCTGTCTTGTTGCGGAGATGTTCCAAGTTTTCCCCCCGGGCCAGGATCCGCTCCACATTCTGGG
TCATAATATTCTTAACTCCCTCCACCTCACTTTGCAGGTTCCGCACACGATCATTTCCTCCACCTTC
ACTGGCTTCCTCCATGTCTCAAAACAAGTCCAAGCCGGTCAGTAAAGTGAATTCGCCTAGTCGGCT
TTCCTCCAAGGTGGCCCTCANTTCACTTCCTGCTTGCTTCAACTTTTANCCTGCCCCCGCCCCCGNG
TACCTTTGGGCCGNTTTANNAACTAGTGGATCCCCCG

| | |
|---|---|
| Sequence 191 | cMhvSD022f04a1 |

CGCACAGTAACAGTAATAGTCAGCCTCATCCTCAACGTGGGCCCCACTGATGGTCAAGGTGACTGT
GGTCCGTGAACTGGAGCCGGAGAATCGCTCAGAGATCCCTGAGGGCCGCTCGCTGTCTTTATACAT
CACTAACACAGGGGCCTGGCCTGGCTTCTGCTGGAACCACCGAGCATCTTTTTTTGCCAGTACCTC
GGCCGGGACCGAGGGTTTGGTGCACCTCGATTTGGAGGAAGTAGGGCAGGGCCCTTATCTGGAAA
GAAGTTTGGAAACCCTGGGGAGAAATTAGTTAAAAAGAAGTGGAATCTTGATGAGCTGCCTAAAT
TTGAGAAGAATTTTTATCAAGAGCACCCTGATTTGGCTAGGCGCACAGCACAAGAGGTGGAAACA
TACAGAAGAAGCAAGGAAATTACAAGTTAGAGGTCACAACTGCCCGAACCAGTTCTAAACAATTA
TTTTTACTAAAATGCATAATTATGTGATAGTTATACATATACCAACCTGTTATGTGAGAACAAAGC
TGANCTGCAAGTANTTCCAAGGCNAGTGAATTAATTACTGGTTGTACCCTCGGGCCGNTCTAGAAC
TAATTGGATNCCCCCGGCTTGCAAGGAATTCGATATTAAAGCTTATTCGAATACCGGCCAACCTNN
AAGGGGGGGNC

| | |
|---|---|
| Sequence 192 | cMhvSD025a09a1 |

CNCGGTGGCGGCCCGAGGTACTGTNTAACTGGATGCTGCCCTGGTTNCTGAAGGCACTTTTCATGA
TGCTGTCCAGGGTCATCAGGGAGACATGTTGAAAGAGCTCCAGACGTGAGTTTTGGGCAATGTGTT
CCTCCCATTTGTTCAGCATCATCCGAACACTCTTAGACATCATGGTGATGAATATTTTCAGAATGCT
GATGTTGAAGCCAGGTTTCACAATCTGGCGGTGCTTTTTCCATTTAGAACCATCCAGGGTCACAAG
TCCTCGACC

| | |
|---|---|
| Sequence 193 | cMhvSD025d09a1 |

GGCGAATTGGAGTTCCCCGCGGTGGCGGCCGAGGTACTCTGCGTTGTTACCACAGGCGATGACAG
CTCCATGTGTGTTATTNNCCCTGAAGACCTTCCAGAGACAAAATGTGGAGGTGGAAGACAGTGAT
ACTGATGACCCTGACCCTGTGTGGATCTAGGCTAACATGTGTTTTTGTGTCTTAGTTTTCAACAAAA
AAGTTTAAAAAGTTAAAATACTAAGTTTATAAAGTTAAAAAGTTACCCCGCGTACCTGCCCG

| | |
|---|---|
| Sequence 194 | cMhvSD025f12a1 |

AGGGCGAATTGGAGCTCCCCGCGGTGGCGGCCGAGGTACTCTGCGTTGTTACCACAGGCGATGAC
AGCTCCATGTGTGTTATTGCCCCTGAAGACCTTCCAGAGACAAAATGTGGAGGTGGAAGACAGTG
ATACTGATGACCCTGACCCTGTGTGGATCTAGGCTAACATGTGTTTTTGTGTCTTAGTTTTCAACAA
AAAAGTTTAAAAAGTTAAAATACTAAGTTTATAAAGTTAAAAAGTTACCCCGCGTACCTGCCCG

| | |
|---|---|
| Sequence 195 | cMhvSD025g04a1 |

CGGCGAATTGGNTTTNCACACGCGGTGGCGGCCCGAGGTACCAAGGAGAAGACTTGAACCAAAAA
CAAACTCTTCAAGTATATTCATTCATTCAACAAAATTTTTGCATGCCTTCTATGTCGTAGGCATTTT
TAGTTCCTGGGGATTTGGACATGGCTAAGTCAGAGAAGGCCATTGCTCACCATGAACACTGTATAC
CAGAAGGAGAGTGGGGAGGAGACAAAAAACAAATAAGACCACTTCAGACAATCAAAGTATCAGT
TAAGAGAATGAAAACAGGCCTGACTCAGTGGCTCACGCCTGTAATCCCAGTACCTGCCCG

| | |
|---|---|
| Sequence 196 | cMhvSD025h04a1 |

CGCGGTGGCGGCCCGCCCGGGCAGGTACAAGGCAAATACTGCTTTATTTTTCCTTCAGCTTTTCTC
AAGCAGAAGAAGTCTCTCACTATAGCCACCACAGCTGGCAATATGCTGGGTCTCACCTGGAGCCG
GAAAGTCTCAGAGTCTCACCCAAGGCCCATGGTATACTACTTGGATATTGCTGCTGGTTATTCAAG
GCCCAAGGGATCTTTAGTCAGCAGGTGACGTATTCCGCAAGGACTGGGTCCTTTCCTTCATGGCAG
CAGGTTCCCTTCTGGCCCAGGGTGTTTCTAAAAATGGTTTCTGGGAGCTAGGAATCCCCACTCATC
AAAGAGGACTTCAATGCAAGACAAAGTCCTCTTTACTCTTCTCCCTCCTCTCCCAAGAGGAAGGAA
GGGTCTCTTTTGGAAGTCAGGAGCTGCATTCCCTGGGGTTGGGGAANGGGTAGTACCTTGGCCGCT
CTA

| | |
|---|---|
| Sequence 197 | cMhvSD025h05a1 |

CGCCCGGGCAGGTACAAGCAGTAATTGATTCACTGGCCTTGGACTACTTGCAGGTCAGCTTGTCTC
ACATAACAGGTTGGTATATGTATAACTATCACATAATTATGCATTTTAGTAAAAATAATTGTTTAG
AACTGGCTTCGGGCAGTTGTGACCTCTAACTGTAATTTCCTTGCTTCTTCTGTATGTTTCCACCTCTT
GTGCTGTGCGCCTAGCCAAATCAGGGTGCTCTTGATAAAAATTCTTCTCAAATTTAGGCAGCTCAT
CAAGATTCCACTTCTTTTTAACTAATTTCTCCCCAGGGTTTCCAAACTTCTTTCCAGATAAGGGCCC
TGCCCTACTTCCTCCAAATCGAGGTGCACCAAACCCTCGGTCC

| | |
|---|---|
| Sequence 198 | cMhvSD026c04a1 |

TTAATACGACTACTATAGGGTTAATTGGAGCTCCCCGCGGTGGCGGCCGAGGTACTTGTTGTTGCT
TTGTTTGGAGGGTGTGGTGGTCTCCACTCCCGCCTTGACGGGGCTGCTATCTGCCTTCCAGGCCACT
GTCACGGCTCCCGGGTAGAAGTCACTTATGAGACACACCAGTGTGGCCTTGTTGGCTTGAAAGCTC
CTTCAGAAGGAGGGGTGGGAACAGAGTTGACCCGAGGGGGCAGCCTTGGGCTGACCTANGACGGT
CAGCTTGGTCCCTCCGCCGAACACCCAAGTGCTACCATCTCCATATGAGCAGCAGTAATAATCAGC
CTCGTCTTCAGCCTGGAGCCCATAGATTGTCAGGGTAGGCNCGTNGTTGCCAGGACTTTGGAGCCA
AGAGAAGNCGAATTAAGAAAACCCCTTGAAGGGGCNCGCTTACTT

| | |
|---|---|
| Sequence 199 | cMhvSD026c09a1 |

CCGCGGTGGCGGCCGAGGTACCTACGCTATCAGGAGGCCCTGAGTGAGCTGGCCACTGCGGTTAA
AGCACGAATTGGGAGCTCTCAGCGACATCACCAGTCAGCAGCCAAAGACCTAACTCAGTCCCCTG
AGGTCTCCCCAACAACCATCCAGGTGACATACCTCCCCTCCAGTCAGAAGAGTAAACGTGCCAAG
CACTTCCTTGAATTGAAGAGCTTTAAGGATAACTATAACACATTGGAGAGTACCTGCCCG

| | |
|---|---|
| Sequence 200 | cMhvSD026c09a1 |

GCTTTTGTTTCCCTTTAAGTGAGNGGTTAAATTGCCGCCGCTTGGGCGTTAATCATGGGT

| | |
|---|---|
| Sequence 201 | cMhvSD026d02a1 |

GCTGTTATGCTCATCATGGCACTTAAGAGATGCTTAACAAACCTTTCCTACAATGTTCCTCAGATTT
TCAGAGCTTATTTGATCTAGCATCTGGTTCCTAAATTCTGAGTCACATCAGAAGCCAAACTTGAAT
GCTTTTGGAAAGAGCTAGCCTCATACCACTTCAAGTTGGGGAAGGGGGAGTACCTCGGCCCGCTCT
AGAAACTAGTG

| | |
|---|---|
| Sequence 202 | cMhvSD026d02a1 |

CGCTTGGCCGTAATCATGGTCATAGCCTGTTTCCTGTGTGGAAATTGTTATCCGCTTCACAATTTCC
ACCACCAACCATAACGAAGCCCGGGG

| | |
|---|---|
| Sequence 203 | cMhvSD026d07a1 |

CCGCGGTGGCGGCCGCCCGGGCAGGTACTTTTTTTGTGATTTTTGAATGCACGTGCGCAGGAAGGG
CTCCTCTTAGAGAAGCAGTCAAACTGTGAAGCACTAAGCTGACCCTGCTTCAAGCAATTTTGTTTT
TACAACTGTTCCTTTCACAAGCAAGCCTTAAAAAAAAAAANNAANTAAAAAANAAAGTACCTCGG
CCCGCTCTAGAACTAGTG

| | |
|---|---|
| Sequence 204 | cMhvSD026d07a1 |

AGCTGTTTCCTGTGTTGAAATTGTTATTCCCGCTCNCCAATTTCCACACAAACANTACCGAAGCCC
GGGGAG

| | |
|---|---|
| Sequence 205 | cMhvSD026d09a1 |

ACACTACTTAGGGCGAATTGGAGCTCCCCGCGGTGGCGGCCGCGGTCTCGGTCACTCGAATAACCC
GACATGGCGTCAATGGTTGCGGTTGGCGGGGAACGAAGTATATAGAAAAGCGTGCGACAAGTCGC
TGGAAATGGCCTCGATGACGGCGAAGCCTTGCGGGGGCGGCAGCGGAGGAAGGACACCGATGAC
ACCAGCCCGAAGCTGCACTACTAGAGACCGGTAGAAATGAATGAGGCCCCCGCGTACCT

| | |
|---|---|
| Sequence 206 | cMhvSD026d09a1 |

CTTGGCCGTTAATCATGGGTCATTAGGCTGTTTTCCTGTGGTGAAAATTGTTATC

| | |
|---|---|
| Sequence 207 | cMhvSD026f02a1 |

AGGTGCAGAAAACTCTCCTCATCTGGACCCGTGACGTCCTTGCAGCCCGAGTTGGCCATATCCCAC
TACGCCCCTGCACTGGAGCCTGAAGCAAAGTGTAAGGAACGGCCAGAGAGCGCAACACTGGGGCC
CACTACCCCGGCGCAAGTGACCCGCCGCCCCCGCGTACCTGCCCGGGCGGC

| | |
|---|---|
| Sequence 208 | cMhvSD026f02a1 |

GCTGTTTCCTGTGTGAAAATTGGTTATCCGCTCACAATTTCCACACAACATTACGAAGCCGGGGGAG

| | |
|---|---|
| Sequence 209 | cMhvSD027a02a1 |

GCTNATTGGAGCTCCCCGCGGTGGCGGCCGAGGTACGCGGGGGAGTCCTTGGAGCGCTGTGTTNTT
TACCGTGGTGGTGACTGGATCCAGGAGGTCGAGAGTCGTTCTTCTCTTTGCACAGACGTGACTCTG
CAGCTCTTTAACGGCGCCCGCTGCTCTCAACCCAGCTTACCCCACGTGGTCCCATGGCGGCGGCCG
CTCTAGAACTAAGTGGATCCCCCGGGCTGCAAGGAAATNCTATATCAAGCTTATCGATACCGTA

| | |
|---|---|
| Sequence 210 | cMhvSD027a10a1 |

CCGCGGTGGCGGCCGAGGTACCCTTATTCGCCTCTTTGACACACAATCCAAGGAGAAACTGGTGG
AGCTGCGCCGAGGCACTGACCCTGCCACCCTCTACTGCATTAACTTCAGCCACGACTCCTCCTTCCT
CTGCGCTTCCAGTGATAAGGGTACCTGCCCGGGCGGCCGCGGTCTCGGTCACTCGAATAACCCGAC
ATGGTGTCAATGGTTGCGGTTGGCGGGGAACGAAGTATATAGAAAAGCGTGCGACAAGTCGCTGG
AAATGGCCTCGATGACGGCGAAGCCTTGCGGGGGCGGCAGCGGAGGAAGGACACCGATGACACC
AGCCGAAGCTGCACTACTAGAGACCGGTTAGAAATGAATGAGGTCCCCGCGTACCTCGGCCGCTC
TAGGAACTAGTGGATCCCCCGGGCNTGCAGG

| | |
|---|---|
| Sequence 211 | cMhvSD027f02a1 |

GGGCGAATTGGAGCTCCCCGCGGTGGCGGCCGCCCGGGCAGGTACAAAGCAGACTGCCCGCAAAT
CGACCGGTGGTAAAGCACCCAGGAAGCAACTGGCTACAAAAGCCGCTCGCAAGAGTGCGCCCTCT
ACTGGAGGGGTGAAGAAACCTCATCGTTACAGGCCTGGTACTGGGAAAAGATCTAATCTGCCGTG
GGCCTGTCGTGCCAGTCCTGGGGGCGAGATCGGGGTAGAAATGCATTTTATTCTTTAAGTTCACGT
AAGATACAAGTTTCAGGCAGGGTCTGAAGGACTGGATTGGCCAAACATCAGACCTGTCTTCCAAG
GAGGCCAAGTCCTGGCTACATCCCAGCCTGTGGTTACAGTGCAGACAGGCCATGTGAGCCACCGC
TGCCAGCACAGAGCGTCCTTCCCCCTGTAGACTAGTGCCGTAGGGAGTACCTCGGCCGC

| | |
|---|---|
| Sequence 212 | cMhvSD027f09a1 |

ACTTAGGGCGAATTGGAGCTCCCCGCGGTGGCGGCCGAGGTACCCACAGCTGGGAGAGAGCTAGT
GAGCTCCAGGGAGGGTCANCTGGGGGAGTTTCACCATTGGCTGTGTCAGCCAATGGCAAGGTGTG
TGAACAGGGAACTCCTGTGTTGAGCATAGAGAGGAANAANATGCNTCCGAGATGGANTTGGGGA
ANGCAAGCACTTGCCGTGTTTGTGTGTCCNGAGACTCGGGCTGNTNATGANGAGCANGAGGGAGC
GTATGAAGATATCANATNTGCAAAGGACAAAACCCCCACCCAATTACAGGACCACTGANCCTNTA
GCTATGGAAGTCTTAANTACAGATTGCCTGGGCCGGGTGGATTTTC

| | |
|---|---|
| Sequence 213 | cMhvSD027g04a1 |

TCCTTAGGGCGAATTGGAGCTCCCCGCGGTGGCGGCCGAGGTACACCAGCGAATTCATACAGGTG
AGAGACCTTATATATGCAATGAATGTGGAAAAGGCTTCATTCAGAAGACGTGTCTCATAGCACATC
AGAGATTTCACACAGGAAAGACGCCCTTTGTGTGCAGTGAATGTGGAAAATCCTGTTCTCAGAAA
ATCAGGTCTCATTAAACATCAAAGAATTCACACAGGAGAGAAACCCTTTGAATGTAGTGAATGTG
GGAAAGCCTTTAGCACAAAGCAAAAGCCCATTGTCCATCAAAGGACTCATACAGGAGAGAGACCC
TATGGCTGTAACGAGTGTGGGAAAGCGTTTGCNGTATATGTCGTGTCTGGTTAAGCATAAGAGAAT
ACACACAAGGGAGAAACAAGAGGCAGCCAAGGTGGAAAAT

| | |
|---|---|
| Sequence 214 | cMhvSD029b07a1 |

CCAGCAGAAGCCAGGCCAGGCCCCTGTGTTAGTGATGTATAAAGACAGCGAGCGGCCCTCAGGGA
TCTCTGAGCGATTCTCCGGCTCCAGTTCACGGACCACAGTCACCTTGACCATCAGTGGGGCCCACG
TTGANNATGAGGCTGACTATTACTGTTACTGTGCGGCCGCCCGGGCAGGTACGCGGGGAGTCGGG
CCGCGCCGCGCCTCAGCTCTGGTTGATGATAATTAGAAGCATGCTTTCCACTGAACTTCCCGACAA
CATTTGTTATGCAGAATGTCTCTGAGTGAGAACTCGGTTTTTGCCTATGAATCTTCTGTGCATAGCAC

| | |
|---|---|
| Sequence 215 | cMhvSD030c12a1 |

ANCAACTAACCGCTCCGTGAACTCCACATCGTTCTCAAATTCTGGGAAGTGTTCCATCTCAATTCC
AACCATGAGGTACCTGCCCGGACCTGCCCGGGCGGCCGCTCTNGAAACTAGTAGGATCCCCCCGG
GGCTTGCATGGAATTNGATATCAAAGCTTTATCCGATACCN

| | |
|---|---|
| Sequence 216 | cMhvSD030f04a1 |

AGGTACTTGTTGTTGCTTTGTTTGGAGGGTGTGGTGGTCTCCACTCCCGCCTTGACGGGGCTGCTAT
CTGCCTTCCAGGCCACTGTCACGGCTCCCGGGTAGAAGTCACTTATGAGACACACCAGTGTGGCCT
TGTTGGCTTGAAGCTCCTCAGAGGAGGGCGGGAACAGAGTGACCGAGGGGGCAGCCTTGGGCTGA
CCTAGGACGGTCAGTTTTGGTCCCTCCGCCGAACACCCAAATGCCATTACTCGAGCCGGCCGCCCG
GGCAGGTACCGCGGGCTGGTGACCTCAGCCAAGAATGAATTCAGGCCATCCGGCTACAAGGCCAA
AAGCTTTNCCCAGCTTANCTACTTTGAACCACCCTGCTTTCTGGNTTTTTCTGGTTTCCACTTGCAA
AAATTGGGANGGGTGTTTTGNTCCTTTTTCCCTTGGGCNTTCCAAACAATTCAAATTTTAAAAA

| | |
|---|---|
| Sequence 217 | cMhvSD030g01a1 |

GGCGAATTGGAGCTCCACTCGCGGTGGCGGCCGAGGTACTGTCCAACTGGATGCTGCCCTGGTGG
CTGAAGGCACACTTCATGATGCTGTCCAGGGTCATCAGGGAGACATGTTGAAAGAGCTCCAGACG
TGAGTTTTGGGCAATGTGTTCCTCCCATTTGTTCAGCATCATCCGAACACTCTCAGACATCATGGTG
ATGAATATTTTCAGAATGCTGATGTTGAAAGCCAAGGGTTTNACAATCTGGCGGGTGNTTTTTT

| | |
|---|---|
| Sequence 218 | cMhvSD030h02a1 |

GGCGAATTGGAGCTCCCCGCGGTGGCGGCCGCCCGGGCAAGGTACATTCTTCTCAGCACCTTAGA
GCCCACTGATGCAGGCATACTGGGAACGACTAAGGACTCACCCAAGCTGGGTCTGCTCATGGTGC
TTCTTAGTATCATCTTCATGAATGGAAATCGGCCAGTGAGGCTGTCATCTGGGAGGTGCTGCGCAA
GTTGGGGCTGCGCCCTGGGATACATCATTCACTCTTTNGGGGACGTGAAGAAGCTCNTCACTGATG
AGTTTTGTGAAGCAAGAANTTACCCTCGGGCCGCTCTAGAAACTAAGTNGGATCCCCCGGGGCTG
CAGGAATTCGATATTCAAGGCCTTATCGGATTACCGTCTNACCCTCGAAGGGGGGGGGGCCCCGG
GTACCC

| | |
|---|---|
| Sequence 219 | cMhvSD031c07a1 |

AGGTACAGGACACAATGCCCCCAGAAAAGTAACAGCCGTCATTTATGCTAGAAAAGGAAGTGTCC
TCCAGAGCATAGAGAAAATAAGTTCCTCTGTTGATGCAACAACTGTTACTTCACAACAGTGTGTTT
TTAGAGACCAAGAACCAAAGATCCATAATGAGATGGCATCAACATCAGATAAAGGTGCCCAAGGA
AGAAATGACAAGAAAGATTCTCAAGGAAGAAGTAATAAGGCATTACATCTGAAGAGTGATGCTGA
ATTTAAAAAGATATTTGGCCTTACTAAGGATTTGAGAGTGTGCCTTACTCGAATTCCTGACCATTTG
ACCTCTGGAGAAGGTTTCGATTCCTTTAGCAGTTTGGTAAAGAGCGGTACCT

| | |
|---|---|
| Sequence 220 | cMhvSD032b02a1 |

CGAGGTACACAAGCTCCTGCATCAGTGCAGGACTCAGTCCCTGAGTGCTGGGCCTGTCACAGACAT
CGCCTTCTTTACTCCCACGCAGCCAGGTTGACAATCACAGACCCTTTCTACAGGGAACCTAAGACA
CCAATTTAACCTGGCCAGGCTGAGCTAGTGGGTCACAAGCTTGAAATCTGAGGTACCTGCCCG

| | |
|---|---|
| Sequence 221 | cMhvSD034b02a1 |

AGGTACCAATGTCTTGGGGGGAGGGAGCCAGCTGATTGTGAGATGTAAGTTTGTGATTCTGAGAT
ANCANCTTTGCAAAAAACTGCAATTTGTCAATTCACCAATATTGATAATGTGCAAGCTTGGTGAGC
TGAGAATATTCCTGAAAACCTTTGTTCCCACTGCGAATTCCTGGGGACAGTTATGAGTTCCTAATG
ACGTCACCACAAAGACATTTTGGAGTGTTTGGTAAAGGCTGTTTCTTTTCAGTGATTGCTGGAAGC
ANATGGGATCAAATAAAAATAGA

| | |
|---|---|
| Sequence 222 | cMhvSD034d09a1 |

AGGTACAGAGTGGACCATCTTATGAGGCCAAAAACCCATGAGTTACCAGATGACCATTCAGATAT
TTGGGTTAAACGATGACAGTTTTCTGGTTTAATCAAGGCACTTGCAAAGAGCTATCTTTGACATGA
CATGAAGTCCCTACGTGTTGTTAGCCATTAATGATGGCATGGTTTTTCTATACCAAGCATTCTATAA
CAAGAACCCAAGCCTGACAGTTTGATCACAAAGTCACTTATAACCCGCGTACCTGCCCGGGCGGC
CGCCCGGGCAGGTACGCGGGGGCCAGCCAAGATGGTTGCCCCCGCAGTGAAGGTTGCCCGAGGAT
GGTCGGGCCTGGCGTTGGGCGTGCGGCGGGCTGTCTTGCAGCTT

| | |
|---|---|
| Sequence 223 | cMhvSD041c11a1 |

ACTATAGGGCGAATTGGAGCTCACCGCGGTGGCGGCCGGGCCCGTGGAGGCCTAGGCTGGCCCTA
GGACCTTCTTGGTTTGCTCCTTGGATTCCCCTTCCCACTCCAGCACCCCAGCCAGCCTGGTACCTCG
GC

| | |
|---|---|
| Sequence 224 | cMhvSD042e09a1 |

TAGGGCNAATTGGAGCTCCCCGCGGTGGCGGCCGCCCGGNCAGGTACCAAAAAACATATTGGTTT
GGCAATGCATCTCCANANCAGGTGATCCTGGCCGTNTGTCCTGGGGACACTGACACCGAGGGNGG
CTGTATCANNTCATAAGAGGCCTCANAGCCTGNGCANANAGTGAGGATGGGGAGAAGTACAGGG
ATCCANGCCATGGNNANACACCCNGAGTTCTGCCTCCTGGACCCACCCCCGCGTACCT

| | |
|---|---|
| Sequence 225 | cMhvSD042e09a1 |

ACCTCGAGGGGGGGCCNGGTCCCAGCTTTTGTTCCCTTTAATGA

| | |
|---|---|
| Sequence 226 | cMhvSD043b06a1 |

NAATTGGAGCTCCCCGCGGTGGCGGCCCGCCCGGGCAGGTACGCGGGGACACCAAACAACTCATT
ACACAAAGAGGTAAGGTCCCAGACCACGCCAAAGCTTCCTGAGACCTCTCCTCATCTGTGCATGG
ACGGATGACCAACTCTGGGGCCCAGGCTGTTGCTTCCCAGTATAATGATGAATCCGCCATAGTCTG
GTGAGTGTAGAGGCTGACTCTGGAGCCCAGGCTGTACCT

| | |
|---|---|
| Sequence 227 | cMhvSD043h11a1 |

GGAGCTCCCCGCGGTGGCGGCCCGCCCGGGCAGGTACTTGGATTACAGGCGTGGACCAGCATGCC
ATGCCTATAGTGATATCTTTAAGTAACCCTCTCTTTTCTTCTTTTGAGCAATTTTTCAAAGCAACAG
GCATTTTATTAAATAAGAAAGTCGATGTGCTTTCCTAATGCCTGTTAATAAAGTAAGGAGCCAAGG
AACCTCTGTGATTTCAATGAAATCCCTCCAGATATTATAGGCTACTTGTTACTGACAAGTATGGCN
GGAACTGCANGTCAAGCTGTGATAGGCAAATAGATCTTGCTGAAGAGGAAGAATG

| | |
|---|---|
| Sequence 228 | cMhvSD044e12a1 |

GGGCGAATTGGAGCTCCCCGCGGTGGCGGCCGCCCGGGCAGGTACGCGGGGCCAGGCGGAAGCC
CGGCTCCGGGCCAGCATCCGAGAGCCCGGACTGGAGAGTCAACTTTTATAACACTGTTACTGGGA
ATACTTGACTTACTAAGCTTTTACTGAACACTTTAATTTTGGGAGTACCT

| | |
|---|---|
| Sequence 229 | cMhvSD044f12a1 |

AGGTACCGCTTTGTANGGGAAGGAGGAGTAAGGATGTCGGAGACCTGTGTCCAGGTGCACCGATG
CCAGACAGACGCTCCCATGTGGCTGAATGGGACCCACCCTGCCCTTGGGGATGGCATCACCAACC
ACACTGCCTGTGCCCATTGGAGTGGCAACTGCTGTTTCTGGAAAACAGAGGTGCTGGTGAAGGCCT
GCCCAGGCGGGTACCTGCCCGGGCGGCCGCCCGGCAGGTACTGTTTCTNAACCTGANCTGCATATT
GGAATCACCTGGGGAGCTTTNACAACTACATGATTCCTAGGACCCATCTCCANAAAGTCCAAAAT
AATTGCTCTGGGTGCAANCTGGACTGTGGGATTTTTAATCCCTTCCCTCCCTGANATTCTAATGTGC
AACCAGTGNNAAGNAACATCATCCTGTNNACCGTTTNCCAAACANGTGTGGATNTGGGCANACAG
GCTTGTCAAAATGCCTTTTCCCANATCCATCCCAAGACAACAAATTCATTANTTTTGGGGCAACTT
CCAAAATNTTACTTTTTTNTCAANTCCAANCCCCATTTTNATTTTTATNGAAGANGGCGTTNTAACA
AATTTAAAAA

| | |
|---|---|
| Sequence 230 | cMhvSD044h04a1 |

CCGGGCAGGTACTTTGAGCAAGGTCCGCAAGCAGGATGCCTGCACTTCTCCAGTCATGCTCCAGCA
CCAGGTCGGAAGCTGTCTACATGCGGGGATGGACCCTGGCATCCTGGGCTCACAAGGATAGGGCC
CTGAATATGGGCNNAGCCGANCNNNCTTGAGANGGNAGCTGCACCCACCCTGAGTGCCTCCCGTG
GTACCT

| | |
|---|---|
| Sequence 231 | cMhvSD045c04a1 |

CCGGGCAGGTACNCGGGGGCTGTANGCTCAAGAGGNACANNTCTGAATGTCTCACCATGGCCTGG
ATNCNTCTCCTGCTCCCCCTCCTAATTCTATGNACAGNNTNTGTGGCCTNCTATGAGCTGACACAG
CCATNCTCAGTGTCAGTGTCTCCGGTAGAGACAGCCAGGATCACCTGCTCAGGAAATGTACCT

| | |
|---|---|
| Sequence 232 | cMhvSD045c04a1 |

GATTNTGAAAATATTCATCACCATGATGTCTGANAGTGTTCGGATGATGCTGAACAAATGGGAGG
AACACATTGCCCAAAACTCACGTNTGGAGCTCTTTCAACATGTCTCCCTGATGACCCTGGACAGCA
TCATGAATGTGCCTTNNCCACCAGGGCANCATNCANTTGGACAGTACCTTGGCCGNTCTANAACTA
TGGATCCCCCGGCTGANGAATTNNANNTCAACTTATNNATCCNNNACTNNAGGGGGGCCCGGNCC
CNACTTTTG

| | |
|---|---|
| Sequence 233 | cMhvSD045c12a1 |

TTGGAGCTCCCCGCGGTGGCGGCCGGCCATGGAGGCTGATGGGGCCGGCGAGCAGATGAGACCGC
TACTCACCCGGGGTCCTGATGAAGAAGCTGTTGTGGATCTTGGCAAAACTAGCTCAACTGTGAACA
CCAAGTTTGAAAAAGAAGAACTAGAAAGTCATAGAGCTGTATATATTGGTGTTCACGTCCCGTTTA
GTAAAGAGAGTCGCCGGCGTCATAGGCATCGCGGACACAAAC

| | |
|---|---|
| Sequence 234 | cMhvSD046e04a1 |

GATTGGAGCTCCCCGCGGTGGCGGCCGAGGTACGCGGGGATGGCTGGCCAGAGGAGGAACGCTTT
GTGTTCTCATCGGAGCTGCATGGGAAGTCTGCATACAGCAAAGTGACCTGCATGCCTCACCTTATG
GAAAGGATGGTGGGCTCTGGCCTCCTGTGGCTGGCCTTGGTCTCCTGCATTCTGACCCAGGCATCT
GCAGTGCANCGAGGTTATGGAAACCCCATTGAAGCCAGTTCGTATGGGCTGGACCTGAACTGCGG
AGCTCCTGGCACCCCAGAGGCTCATGTCTGTTTTGACCCCTGTCAGAATTACACCCTCCTGGATGA
ACCCTTCCGAANCACAGAGAACTCAGCAGGGTCCCAGGGGTACGATAAAAACATGAGCGGCTGGT
ACCTGCCCGGGCGGNCGCCCGGGCANGTACTNANGTGTAAAGGGATTTATATGGGGACNTTGGCC
NATTTNCNGGTGTTGNCNGTTNCTCTTTTTAAGCTTATACTCATGAATCTTGTNTTAANCTTTTGAA
GGCANACTGCCNAAATNCTGGANAAATANNAGNTNGNNAANNNNGGGGGTTTTTTTT

| | |
|---|---|
| Sequence 235 | cMhvSD046g04a1 |

GGTGGCGGCCGCCCGGGCAGGTACCTCAGAAGCAAACCCAGTTCCTGCACACAGAAACCCCATTC
AGGCTCCTACTGCACTGAGAAGCACGTGTTCTCCATTTCCCTGGGGGAGACCATTGTATTGGGCAG
TTNGGAACAAAACACCATGGACTGGGA

| | |
|---|---|
| Sequence 236 | cMhvSD047e10a1 |

TTAGGGCGAATTGGAGCTCCCCGCGGTGGCGGCCGCACAAAAACCAATCTACCTGATGAAAACTC
CGTTCCCTTCTCGCCAGAAACATAAAATGCGATGGAGCTACGGCCACCGCTGCCGAGACAAAATG
GCGCCCCCCGCGTACCT

| | |
|---|---|
| Sequence 237 | cMhvSD048e11a1 |

TGGAGCTCCCCGCGGTGGCGGCCGNCNNNCCGGGTNCTCACCAAAATTGGGCCTGAGAAATTTGT
TATATCCTGCTGNGAGGTTCTCAAAGCCAGGCANGGAAAGCTTGTCACTTCTGCCGACCTCGACGT
TGAACTGACTCCTNTGGATGCACATCCTCTCAGTGAAGAGACTCANACACACGAAGGCCAAGTGG
AGGCTGCNNTTCATGTTGAGAAGCTGCTCACACCNANGNNCTGAAGTAAGAATCACNATGTANTT
NTTGAGGCTCTGTTAGGGCAAGTCCTTNAGGCCTACANGCAAGACTTCCAGGCAAGGCACGGTCTT
CTGGGTCCCCAGGGTTCTNCTCATNCTCAGCCCTGTCCCCTNNNATGTGGACACGCANCCACCCTC
AGATGGAGTGGCTCTCTGGGAAAGAATGGAGCTGCTAAACCTGTCTTGGCTCCANCCATGCAGGT
AAGGGGAGGGATTGCTTGGACGCTTGGCCTTGCACCCTGAGGGAGCTGGGAGCCANGAGGGACTC
ATATGGAAGGGCAGANAAAANANCTTANTGNNNGNTACCTGCCCCGGGCCGGCCNTGAACCATTT
ACTGTCGGTGTATTTAAACTGCACTTGGTAGACAACAAGCCTCGTGCTATTGCTCAAGGCCACTGC
TTCCAACTCAGGACCTGCTCTGCTTTGACCTCGGCCCTCTANAACTATGGATCCCCCGGCTGCANG
AATTCATTCAACTTATCGATTCCGTCGACNTCNAGGGGGGGCC

| | |
|---|---|
| Sequence 238 | cMhvSD053f10a1 |

TCACTATAGGGGCGAATTGGGAGCTCCACCGCGGTGGGCGGCCCGAGGTACAGCACCCGCTTGGC
TGTGCTGAGCAGCAGCCTGACCCATTGGAAGAAGCTGCCACCGCTGCCGTCTCTTACCAGCCAGCC
CCACCAAGTGCTGGCCAGTGAGCCCATCCCGTTCTCTGATTTGCAGCAGGTCTCCAGGATAGCTGC
TTATGCCTACAGTGCACTTTCTCAGATCCGTGTGGACGCAAAAGAGGAGCTGGTTGTCGACCAGAC
ACTATTTCAGCTAAAACCCCAGCTCGAAGACCAAAGAAGTGGGTTGGCTTGTCTCTGACAAGTCAC
GCTTTTGATTCTTTTACNGNCTTTGTGGGACACAAAGATGGGTGGAGATGGCTCANAAGTTGGGAG
CTGCTCTCCAGGTTGGGGAGGCACTGGTCTGGACCAAACCAGTTAAAGATCCCAAATCAAAACAC
CAGACCACTTTAACCAAGCAAACCTGCCAGTTTCCAGCAACCTNTGGGCTCTAATCAAAGCTTCTA
GGACAGGCAATGTCTTTAGCAGCTGNATACAAGGACGCTTCCNTTAAGTAGNAACCATNCAAGAG
CTTCCATGAAAGACCTTGGCAAGGTACCCTGCCCGGGCCGGGCGGTTCTAAAAACTNGTGGATTCC
CCCGGGGCCGGAAGGAATTCNATTTAAAAGCTTATTNGANACCCGCCNANCCTTGAAGGGGGGGGG

| | |
|---|---|
| Sequence 239 | cMhvSD054a11a1 |

CGCGGTGGCGGCCGAGGTACAGGAGGCCCGACAATTTGGTGACCAAGTGATGGCAGGCCACTCAG
CTTTGAGTAGCCATGTCCGCCACAGGCCCTGCGGCACATCTCANCTCCCTGGGTGCAGAATTCTGA
CATCATGGCCTTCATGCCCGTGCTCAGTGCGTGGAGCTGTGAGAACATGGAGGGGGGTTGGGCGG
TGTTAGGGGGCCTCCACCATAGGGGACCAACCCTGTGCACCACTTACTGAGCATCTACTCATGCCC
AGCTCAACTCTGAGGTCCCGCGTCCTGCCGGGCGGCCGCTCTA

| | |
|---|---|
| Sequence 240 | cMhvSD054e05a1 |

CCGGGCAGGTACCCAGGGAACAAATGCTACTGGGACTCCACACCTACCTAAGAAGCAGCTCTACC
CAGACTCCACATGGCTCTCTGTTTTGGTCTGGAGACCCCAGCTGGGGTATCTCCTGAGCCCAGGGA
TTCAAAGGTTCGTGGCAGAAATATGCATCCCACGGGACTCTCACTCACTCACCATTTTCTTGTAGG
GGGATTCCCCTGGGTCTGTGCCACTCCTGGGTGAATGGTTGATCTGTCTCACTCTTCTCCGTGATCC
GAAGGTCACACTATGTCACTGATGAATCCTTATGTGTCCACCTGGATGTTCCGGTTGAAGAGCTAG
TGTCTCACCACTCTTTCTGCTATTTGTGAGAGTGGCACACACTAGCTGCTTCTAGTCAACCATCTTG
GCCCCACCTCACTCACTTTTCTCAAGTAATCAAAGACCAGAAAGGATGTCCTTTACAAGAGCAGAT
CCCCCAAAATGTAAGAGTTCACTGAAAAGGTGGGAGCTCAAACCAAGAGAGGACCTATCTCGCAG
CATAAAGACAACTTGTACCTCGGCCGCTCTAGAACTA

| | |
|---|---|
| Sequence 241 | cMhvSD054g09a1 |

AGGTACAGTGTCTCCGTCCCGCGGAAAAAGAAGCCTCTGAACCCGCGCCGGCCCGCAGCCCCCGT
GCCTTCCGGCCGCCCGGGCAGGTACGCGGGGGCCGCGGAGACAAAGATGGCTGCGAGAGTCGGC
GCCTTCTCAAGAATGCCTGGGACAAGGAGCCAGTGCTGGTCNGTGTCCTTCGTCGTCGGGGGCCTC
GCTGTAATTCTACCCCCATTGAGCCCCTACTTCAAGTACCT

| | |
|---|---|
| Sequence 242 | cMhvSD054g09a1 |

AGTGAACTAACTCACATTAANTTGCNTTGGCGCCTCACTGGCCGCTTTTCAAGTNCNGGNAAACCT
GNTCNTGCCAGGCTGGCANTTAATTGAAATCGGGCCAAACGCCCCCGGGGAGAAGGCGGTTTTGC
GTATTTGGGCGGCTNTTTCCGC

| | |
|---|---|
| Sequence 243 | cMhvSD054h08a1 |

ACCGCGGTGGCGGCCCGAGGTACATGACGGGATTTCACTATGTTGGCCAGGCTGGTCTCAAATTCC
TGACCTCGTGACCCACGTGCCTTGGCCTGCCAACATGCTGGGATTGCAGGTGTGAGCCACCGCGCC
CGGCCCCAACTTCTCCTAATGTTGCTATTTTGATCTTATTTTTTAAATCATGAATGTTCTCAATGAC
ATCTAGAATGGTGAATCCTTTCCAGTAGGTTTTCAATTATTTTGCCCAGATCCATCAAAGGAATCA
CTTTCTAGAGAAGTTATAGCTTTATGAAATATATTTTTAAGTGATAAAGACTTGAAAGTTGCAATT
ATTCTTTGATCCAAGGGCACCAAGAATGAATGTTGGGTTAGTAGGCATGAAAACAATATTCAGCTC
TTTGTACCTGCCCG

| | |
|---|---|
| Sequence 244 | cMhvSD054h09a1 |

GGAGCTCCACCGCGGTGGGCGGCCGAGGTACAAGCAGTAATTGATTCACTGGCCTTGGGCTACTT
GCAGGTCAGCTTGTCTCACATAACAGGTTGGTATATGTATAACTATCACATAATTATGCATTTTAGT
AAAAATAATTGTTTAGAACTGGCTTCGGGCAGTTGTGACCTCTAACTGTAATTTCCTTGCTTCTTCT
GTATGTTTCCACCTCTTGTGCTGTGCGCCTAGCCAAATCAGGGTGCTCTTGATAAAAATTCTTCTCA
AATTTAGGCAGCTCATCAAGATTCCACTTCTTTTTAACTAATTTCTCCCCAGGGTTTCCAAACTTCT
TTCAGATAAGGGCCCTGCCCTACTTCCTCCAAATCGAGGTGCACCAAACCCTCGGTCCCGGCCGTG
CTTCTGCTATGGCGAAGGAGCCCTCGGCCTTCAACCACTGTGCCCACGCCTACCGGTTTTCTGGGG
ATGTTGCCACCACCTCTGAAGAGTGAAACCAAGCTTTTCATGCANGAAGAGCCAGGTGCTGGGGG
GCTTC

| | |
|---|---|
| Sequence 245 | cMhvSD055d06a1 |

TCTGAATGATCGCGTTGCTCGAGCTGCCGTTGGAAGCTTAGAAGCAGGTGCTACCGTGCTAGATAC
AAAGCGATCTATTTAAAAGCCCTCTGTCACGCACGCACACTTACTGACGAATCTTCTGGCTCTCTC
CTACCCCGCCCGGTGGCGGATTCCGGAATTGGTTCAAAAGGCCTTGATCCCGAACACCCAGGACA
GAGACAGAGTACCT

| | |
|---|---|
| Sequence 246 | cMhvSD055d10a1 |

CGAGGTACAAGCAGTAATTGATTCACTGGCCTTGGACTACTTGCAGGTCAAGCTTGTCTCACATAA
CAGGTTGGTATATGTATAACTATCACATAATTATGCATTTTAGTAAAAATAATTGTTTAGAACTGG
CTTCGGGCAGTTGTGACCTCTAACTGTAATTTCCTTGCTTCTTCTGTATGTTTCCACCTCTTGTGCTG
TGCGCCTAGCCAAATCAGGGTGCTCTTGATAAAAATTCTTCTCAAATTTAGGCAGCTCATCNAGAT
TCCACTTCTTTTTAACTAATTTCTCCCCAGGGTTTCCAAACTTCTTTCCAGATAAGGGCCCTGCCCT
ACTTCCTCCAAATCGAGGTGCACCAAACCCTCGGTCCCCGGCCGNTCTAAGAACTAATTGGATCCC
CCGGGCTGGCAGGAATTCGATATCCAAGCTTAATCGATCCCGTCGNACCTCGAGGGGGGGGCCC

| | |
|---|---|
| Sequence 247 | cMhvSD055g05a1 |

CGAGGTACTCTGCGTTGTTACCACAGGCGATGACAGCTCCATGTGTGTTATTGCCCCTGAAGACCT
TCCAGAGACAAAATGTGGAGGTGGAAGACAGTGATACTGATGACCCTGACCCTGTGTGGATCTAG
GCTAACATGTGTTNTTGTGTCTTAGTTTTCAACAAAAAAGTTTAAAAAGTTAAAATACTAAGTTTA
TAAAGTTAAAAAGTTACCCCGCGTCCTGCCCG

| | |
|---|---|
| Sequence 248 | cMhvSD055g05a1 |

ATCATGGNCATAGCTTGTTTCTGNTGTNAAATTGTTATCCGCTTCACAAATTCCCACACAAACATA
CNNAGCCCGGGAAGCATAAAAGTGTAAAGGCC

| | |
|---|---|
| Sequence 249 | cMhvSD059a06a1 |

GGGCGAATTGGAGCTCACCGCGGTGGCGGCCCGAGGTACGCGGGGATGCTGCGCCTCTCCGAACG
CAACATGAAGGTGCTCCTTGCCGCCGCCCTCATCGCGGGGTCCGTCTTCTTCCTGCTGCTGCCGGG
ACCTTCT

| | |
|---|---|
| Sequence 250 | cMhvSD059b04a1 |

NATTGGAGCTCCCCGCGGTGGCGGCCGAGGTACAAGTTGTCTTTATGCTGCGAGATAAGTCCTCTC
TTGGTTTGAGCTCCCACCTTTTCAGTGAACTCTTACATTTTGGGGGATCTGCTCTTGTAAAGGACAT
CCTTTCTGGTCTTTGATTACTTGAGAAAAGTGAGTGAGGTGGGGCCAAGATGGTTGACTAGAAGCA
GCTAGTGTGTGCCACTCTCACAAATAGCAGAAAGAGTGGTGAGACACTAGCTCTTCAACCGGAAC
ATCCAGGTGGACACATAAGGATTCATCAGTGACATAGTGTGACCTTCGGATCACGGAGAAGAGTG
AGACAGATCAGCCATTCACCCAGGAGTGGCACAGACCCAGGGGAATCCCCCTACAAGAAAATGGT
GAGTGAGTGAGAGTCCCGTGGGGATGCATATTTCTGCCACGAACCTTTGAATCCCTGGGCTCANGA
GATACCCCAGCT

| | |
|---|---|
| Sequence 251 | cMhvSD059b07a1 |

ATTGGAGCTCCCCGCGGTGGCGGCCGCCGGGCAGGTACAAGCAGTAATTGATTCACTGGCCTTGG
ACTACTTGCAGGTCAGCTTGTCTCACATAACAGGTTGGTATATGTATAACTATCACATAATTATGC
ATTTTAGTAAAAATAATTGTTTANAACTGGCTTCGGGCAGTTGTGACCTCTAACTGTAATTTCCTTG
CTTCTTCTGTATGTTTCCACCTCTTGTGCTGTGCGCCTAGCCAAATCAGGGTGCTCTTGATAAAAAT
TCTTCTCAAATTTAGGCAGCTCATCAAGATTCCACTTCTTTTTAACTAATTTCTCCCCAGGGTTTCC
AAACTTCTTTCCAGATAAGGGCCCTGCCCTACTTCCTCCAAATCGAGGTGCACCAAACCCTCGGTCC

| | |
|---|---|
| Sequence 252 | cMhvSD059c11a1 |

AATTGGAGCTCCCCGCGGTGGCGGCCGCCCGGGCAGGTACCACATGCCTGTAATCCCAGCTACTTG
GAAGCTGAGGCAGGAGAATCTCTTGAACTTGGAAGGCGGAGGTTGCAGTGAACCAAAATCACGCC
ACAGCACTCCAGCCTGGGAGACAGAGCAAGGCTTAGTTTTAAAAAAAAAATCAAATATTGTGTGA
TTCTGTTTATAGGAAATATTCANAATTGGTAAGTCCATAAGGACAAAAACCAGATTGACAGGGGC
TGAGATGAAAAAGAGAATGGGGTATGGGGAGTGACAGCTTGATAGGTATGGGTTTTGTTGGGGGG
AGATAATGAAAACATTTGGAACTAGGAGAATCACCTGACATCAGGAGTTCAAGACCACTGAACTC
GAACCTGGGTGACAGANTGAGACTCCGTCTCAAAAAAAAAAAAAATGTTTGGAACTANATGGTGG
TGGTTGTACCT

| | |
|---|---|
| Sequence 253 | cMhvSD059c12a1 |

TTGGAGCTCCCCGCGGTGGCGGCCGCCCGGGCAGGTACTGTGGGTGGGACAAAATATTTTTTCAGA
TTTTTAGACTTGGAATATGATTCCTGCTGTCTAGCAGAATAAGAAGAAGATAATGGCAGGAGGAC
AGCACGGACTAAACTCCAAGCANAAAAAAACAAAAAGATCAAATTTAAGACCTTTTTGGTGAGCC
CGTTTTAATCCTGGTCCTACTCTGTCCCAAATTTCTACATCAAGACTGCCTGTCTGTGGAAACCACG
GGT

| | |
|---|---|
| Sequence 254 | cMhvSD059d04a1 |

GGCNGATTGGAGCTCCCCGCGGTGGCGGCCGAGGTACAAGTTGTCTTTATGCTGCGAGATAAGTCC
TCTCTTGGTTTGAGCTCCCACCTTTTCAGTGAACTCTTACATTTTGGGGGATCTGCTCTTGTAAAGG
ACATCCTTTCTGGTCTTTGATTACTTGAGAAAAGTGAGTGAGGTGGGGCCAAGATGGTTGACTAGA
AGCAGCTAGTGTGTGCCACTCTCACAAATAGCAGAAAGAGTGGTGAGACACTAGCTCTTCAACCG
GAACATCCAGGTGGACACATAAGGATTCATCAGTGACATAGTGTGACCTTCGGATCACGGAGAAG
AGTGAGACAGATCAGCCATTCACCCAGGAGTGGCACAGACCCAGGGGAATCCCCCTACAAGAAAA
TGGTGAGTGAGTGAGAGTCCCGTGGGATGCATATTTCTGCCACGAACCTTTGAATCCCTGGGCTCA

| | |
|---|---|
| Sequence 255 | cMhvSD059d06a1 |

AATTGGAGCTCCCCGCGGTGGCGGCCCGAGGTACATCATTTCCAGAGCAGGCACTGGCAGCGAGA
TAGGGTTGGAGGAGAAGTAGCGCCGGGACTTCCGGATGGCAAACTTCTCTGTGGGTAGAGATTTC
CCAGCAATCTTGAGCTTCAGGCCTGGACAGCTCGAAATAATTCCACTTCGTCGTCCCCGAACGGCT
TGTGGTCCTCCTTCCCAAACATGCTGAGGTAGGCGGCCTTCATGTAAATGTAGGTGGCCTTTTTAA
GTCAGATCATGTCAGTTCCTTCTGGAAATCTGGTTATATTCCATCACACTCAGGAGACATCTCCTAC
AATTTCCTTGACACCTGCAGCACTCCAGCCACACGACGGCCTCAGGGCGGTTCCCAGGACACATCA
AACACACTCCTGCCCTGCTGTGCCCTGCCCANCTCCCTCTCCCCGCGTACCTGCCCG

| | |
|---|---|
| Sequence 256 | cMhvSD059d10a1 |

TAGGGCGAATTGGAGCTCCCCGCGGTGGCGGCCGCCCGGGCAGGTACCCAGGGAACAAATGCTAC
TGGGACTCCACACCTACCTAAGAAGCAGCTCTACCCAGACTCCACATGGCTCTCTGTTTTGGTCTG
GAGACCCCAGCTGGGGTATCTCCTGAGCCCAGGGATTCAAAGGTTCGTGGCAGAAATATGCATCC
CACGGGACTCTCACTCACTCACCATTTTCTTGTAGGGGGATTCCCCTGGGTCTGTGCCACTCCTGGG
TGAATGGTTGATCTGTCTCACTCTTCTCCGTGATCCGAAGGTCACACTATGTCACTGATGAATCCTT
ATGTGTCCACCTGGATGTTCCGGTTGAAGAGCTAGTGTCTCACCACTCTTCCTGCTATTTGTGAGAG
TGGCACACACTAGCTGCTTCTAGTCAACCATC

| | |
|---|---|
| Sequence 257 | cMhvSD059g02a1 |

GGGCNAATTGGAGCTCCCCGCGGTGGCGGCCGAGGTACTGTCCAACTGGATGCTGCCCTGGTGGC
TGAAGGCACACTTCATGATGCTGTCCAGGGTCATCAGGGAGACATGTTGAAAGAGCTCCAGACGT
GAGTTTTGGGCAATGTGTTCCTCCCATTTGTTCAGCATCATCCGAACACTCTTAGACATCATGGTGA
TGAATATTTTCAGAATGCTGATGTTGAAGCCAGGTTTCACAATCTGGCGGNGCTTTTTCCATTTAGA
ACCATCCAGGGTCACAAGTCCTCGACCAACCCAGGATTCAAGGATTTTGTGGCTAACAGCACTTTT
GGGATCTTGTCTTTTCAGGAGAATCTTGACATAGTCTGGGTCATGGATATTGAAGAACATCGTAAA
GGGTCCAACCCACAAGGGAACGGCACATGGGTATTTTTCCATCAGCTCAGGATCACCTCAAACTCT
TTTACTGGGTAAGAC

| | |
|---|---|
| Sequence 258 | cMhvSD060a05a1 |

GCGAATTGGAGCTCCACCCGCGGTGGCGGCCCGAGGTACAAGCAGTAATTGATTCACTGGCCTTG
GACTACTTGCAGGTCAGCTTGTCTCACATAACAGGTTGGTATATGTATAACTATCACATAATTATG
CATTTTAGTAAAAATAATTGTTTAGAACTGGCTTCGGGCAGTTGTGACCTCTAACTGTAATTTCCTT
GCTTCTTCTGTATGTTTCCACCTCTTGTGCTGTGCGCCTAGCCAAATCAGGGTGCTCTTGATAAAAA
TTCTTCTCAAATTTAGGCAGCTCATCAAGATTCCACTTCTTTTTAACTAATTTCTCCCCAGGGTTTCC
AAACTTCTTTCCAGATAAGGGCCCTGCCCTACTTCCTCCAAATCGAGGTGCACCAAACCCTCGGTCC

| | |
|---|---|
| Sequence 259 | cMhvSD061a11a1 |

AGGTACAGGACATTCCTCTGCTCCTATTGCCCCTGTTTCCGTTCTTTTCACACTGTCTGTGGGTGCT
GTGCCCTGTTGGAACTCTCTTTAACGTCTTACGTTGGAGCCGCTAACCTTCCCCAGGTGTTTGCTTC
ATTGCTTTCACAGGGAAAGAATTACTCGTCCCACTGACGAGTTCTATGTATGTCCCTGGGAAGCTG
CATGATGTGGAACACGTGCTCATCGATGTGGGAACTGGGTACCTGCCCG

| | |
|---|---|
| Sequence 260 | cMhvSD061e08a1 |

GCGAGGTNCTNTNCGNNGTTNCCACACGCGATGACAGNTCCATGTGTGTTATTGCCCCTGAAGACC
TTCCAGAGACAAAATGTGGAGGTGGAAGACAGTGATACTGATGACCCTGACCCTGTGTGGATCTA
GGCTAACATGTGTTTTTGTGTCTTAGTTTTCAACAAAAAAGTTTAAAAAGTTAAAATACTAAGTTT
ATAAAGTTAAAAAGTTACCCCGCGTACCTGCCCG

| | |
|---|---|
| Sequence 261 | cMhvSD061g11a1 |

CCGGGCAGGTACTGTCCAACTGGATGCTGCCCTGGTGGCTGAAGGCACACTTCATGATGCTGTCCA
GGGTCATCAGGGAGACATGTTGAAAGAGCTCCAGACGTGAGTTTTGGGCAATGTGTTCCTCCCATT
TGTTCAGCATCATCCGAACACTCTCAGACATCATGGTGATGAATATTTTCAGAATGCTGATGTTGA
AGCCAGGTTTCACAATCTGGCGGTGCTTTTTCCATTTAGAACCATCCAGGGTCACAAGTCCTCGAC
CAACCCAGGATTCAAGGATTTTGTGGCTAACAGCACTTTTGGGATCTTGTCTTTTCAGGAGAATCTT
GGCATAGTCTGGGTCATGGACACTGAAGAACATCGTAAAGGGTCCAACCCACAAGGGAACAGCAC
ATGGGTATTTTTCCATCAGCTTATGATACCCCTCAAACTCCTTTACTGGGTAAAAC

| | |
|---|---|
| Sequence 262 | cMhvSD061h01a1 |

NAATTGGAGCTCCCCGCGGTGGCGGCNCGAGGTACCTGTGGCAGCCCTTCTTCAGACACGGCTACT
TCTGCTTCCACGAGGCTGCTGACCAGAAGAGGTTTAGTGCCCTCCTGAGTGACTGCGTCAGGCATC
TCAATCATGATTACATGAAGCAGATGACATTTGAAGCCCAGGCCTTTTTAGAAGCTGTGCAATTCT
TCCGACAGGAGAAGGGTCACTATGGTTCCTGGGAAATGATCACTGGGGATGAAATCCAGATCCTG
AGTAACCTGGTGATGGAGGAGCTCCTGTCCACTCTTCAGACAGACCTGCTGCCTAAGATGAAGGG
GAAGAAGAATGGCAGAAAGAGGACGTGGCTCGGTCTCCTCGAGGAGGCCTACACCCTGGTTCAGC
ATCAAGTTTCAGAAGGATTAAGTGCCTTGAAGGAGGAATGCANAGCTCTGACAAAGGGCCTGGAA
GGAACGATCCGTTCTGACATGGATCANATTGTGAACTCAAAGAACTATTTAAT

| | |
|---|---|
| Sequence 263 | cMhvSD062c05a1 |

GCNAATTGGAGCTCCCCGCGGTGGCGGCCCGAGGTACTCTGCGTTGTTACCACAGGCGATGACAG
CTCCATGTGTGTTATTGCCCCTGAAGACCTTCCAGAGACAAAATGTGGAGGTGGAAGACAGTGAT
ACTGATGACCCTGACCCTGTGTGGATCTAGGCTAACATGTGTTTTTGTGTCTTAGTTTTCAACAAAA
AAGTTTAAAAAGTTAAAATACTAAGTTTATAAAGTTAAAAAGTTACCCCGCGTACCTGCCCG

| | |
|---|---|
| Sequence 264 | cMhvSD062d01a1 |

ATTGGAGCTCCCCGCGGTGGCGGCCGAGGTACACTTCCCGGGGAACCACCCACTGGGCTGCAATC
TCCCAGGGAGACTGCAAGGTATGGTCCAGCTTGGGTGCCAGCTCCACCCGCAAGCCAGTCATCATT
CGGTGAAAGGCCCTCTGGTCCTCCCGGTTGGCAGCTGATGTATCTAAGTTGTCAATCAGGAAAACT
TTGGTGAAGATAAAAATGACAAGGAGAATTGCTAACAGCACGACTCGCTGCTTTAGCTTCATGTTG
ACCTCTTTTCCTTCTCCTCTGACCCACTCTTGCTCATGTATTAAGGAGAGCTGGTGGTGATGGTTAG
CAAGGAGATTCCATGATTATACACATTGGTCCATTTCTTCACTGATGCACCTTCCACAGTTCCTTCC
TCCATACGCAAACACAGACTGGCAATTCACAAGTAAATGCAAGGTTTTCAATATCCAACAGTTTGT
AGTCATGAAAAAAAAGTCAAAAGTAAAACACTCCGTACCTGCCCG

| | |
|---|---|
| Sequence 265 | cMhvSD062e01a1 |

CTCCCCGCGGTGGCGGCCCGCCCGGGCAGGTACGCGGGGTAACTTTTTAACTTTATAAACTTAGTA
TTTTAACTTTTTAAACTTTTTTGTTGAAAACTAAGACACAAAAACACATGTTAGCCTAGATCCACAC
AGGGTCAGGGTCATCAGTATCACTGTCTTCCACCTCCACATTTTGTCTCTGGAAGGTCTTCAGGGG
CAATAACACACATGGAGCTGTCATCGCCTGTGGTAACAACGCAGAGTACCT

| | |
|---|---|
| Sequence 266 | cMhvSD062g11a1 |

TAGGGCGAATTGGAGCTCCCCGCGGTGGCGGCCGGGACCCGAGGGTTTGGTGCACCTCGATTTGG
AGGAAGTAGGGCAGGGCCCTTATCTGGAAAGAAGTTTGGAAACCCTGGGGAGAAATTAGTTAAAA
AGAAGTGGAATCTTGATGAGCTGCCTAAATTTGAGAAGAATTTTTATCAAGAGCACCCTGATTTGG
CTAGGCGCACAGCACAAGAGGTGGAAACATACAGAAGAAGCAAGGAAATTACAGTTAGAGGTCA
CAACTGCCCGAAGCCAGTTCTAAACAATTATTTTTACTAAAATGCATAATTATGTGATAGTTATAC
ATATACCAACCTGTTATGTGAGACAAGCTGACCTGCAAGTAGTCCAAGGCCAGTGAATCAATTACT
GCTTGTACCTGCCCG

| | |
|---|---|
| Sequence 267 | cMhvSD065d05a1 |

CCGCGGTGGCGGCCCGAGGTATAATGCCAGGAAGATGAATGTGCGTTAATGTTGCTGGAACATGG
CACTGATCCAAACATTCCAGATGAGTATGGAAATACCACTCTACACTACGCTATCTACAATGAAGA
TAAATTAATGGCCAAAGCACTGCTCTTATACGGTGCTGATATCGAATCAAAAAACAAGCATGGCCT
CACACCACTGCTACTTGGTGTACCTGCCCGGGCGGCCGCCCGGGCAGGTACGCGGGACCCAAAAA
CCACACCCCTCCTTGGGAGAATCCCCTAGATCACAGCTCCTCACCATGGACTGGACCTGGAGCATC
CTTTTCTTGGTGGCAGCAGCAACAGGTGCCCACTCCCAGGTTCAGCTGGTGCAGTCTGGAGCTGAG
GTGAAGAAACCTGGGGCCTCAGTGAAGGTCTNCTGCAAGGCTTCTGGTTACACCTTTACCAGCAAT
GGGTATCAGCTGGGTGCGACAGGCCCCTGGACAAGGGCTTGAGTGGATGGGATGGGATCANCGCT
TACAATGGGTAACACAAACTACNCACAAGAANCTNCAGGGCAGAGTCACCATGACCACAGACAC
ATNCACNANCACANNCTACATGGGAGCTNNNGGAGCCTGNAATCTTACGACC

| | |
|---|---|
| Sequence 268 | cMhvSD067d10a1 |

CCGGGCAGGTACAATGCCTTGAACATCGTCCTGCTTCCCAGTGGGTTCAGACCTCACCTCTCAGGG
AGCGACCTGGGCAAAGACAGAGAAGCTCCCAGAAGGAGAGATTGATCCATGTCTGTTTGTAGGAC
GGAGAAACCGCTTGGGTAACTTGNTCAAGATATGATCGCATGTTGCTTTCTAAGAAAGCCCTGTAT
TTTGTGATTGTCTTTTTTTTTTTTAAGATGCTTTCATTTTGCCAAAATAAAACAGATAATGTTNAAA
AAAAAAANNAAAAANTCAAAAATNAANGTGCCNGGGNCNCTCTANAACTNGNGGNTCCCCCGGG

| | |
|---|---|
| Sequence 269 | cMhvSD067g06a1 |

AATTGGAGCTCCCCGCGGTGGCGGCCGCCCGGGCAGGTACGCGGGGTAACTTTTTAACTTTATAAA
CTTAGTATTTTAACTTTTTAAACTTTTTTGTTGAAAACTAAGACACAAAAACACATGTTAGCCTAGA
TCCACACAGGGTCAGGGTCATCAGTATCACTGTCTTCCACCTCCACATTTTGTCTCTGGAAGGTCCT
CAGGGGCAATAACACACATGGAGCTGTCATCGCCTGTGGTAACAACGCAGAGTACCT

| | |
|---|---|
| Sequence 270 | cMhvSD069f05a1 |

AGGTACAAGCAGTAATTGATTCACTGGCCTTGGACTACTTGCAGGTCAGCTTGTCTCACATAACAG
GTTGGTATATGTATAACTATCACATAATTATGCATTTTAGTAAAAATAATTGTTTAGAACTGGCTTC
GGGCAGTTGTGACCTCTAACTGTAATTTCCTTGCTTCTTCTGTATGTTTCCACCTCTTGTGCTGTGCG
CCTAGCCAAATCAGGGTGCTCTTGATAAAAATTCTTCTCAAATTTAGGCAGCTCATCAAGATTCCA
CTTCTTTTTAACTAATTTCTNCCCAGGGTTTCCAAACTTCTTTCCAGATAAGGGCCCTGCCCTACTT
CCTCCAAATCGAGGGTGCACCAAACCCTCGGTCC

| | |
|---|---|
| Sequence 271 | cMhvSD069g08a1 |

CCGGGCAGGTACGCGGGTCATGGATCGAAGACTCATGCAAGATGATAATCGTGGCCTTGAGCAAG
GTATCCAGGATAACAAGATTACAGCTAATCTATTTCGAATACTATTAGAAAAAAGAAGTGCTGTTA
ATACGGAAGAAGAAAAGAAGTCGGTCAGTTATCCTTCTCTCCTTAGCCACATAACTTCTTCTCTCA
TGAATCATCCAGTCATTCCAATGGCAAATAAGTTCTCCTCGCCTACCCTTGAGCTGCAAGGTGAAT
TCTCTCCATTACAGTCATCTTTTGCCTTGTGACATTCATCTGGTTAATTTGAGAACAANACAAGTCA
AAAGGTNGGCAATGGGGCACTTCCAAATGAAGGCAGCCTTGGATCCTCCACAAGAAAAGGGTTTT
GATTTGTCGGTTTCTTCTAAGCAAAGGGNACAAGGGGTTGGTTTTTGGTTCTACTACCTCAGGGGG
AAAAGGAATATTTGGTACCCTTTGGGCCCGCNTCTTAGAAACTTAGTNGGAATCCCCCCCCG

| | |
|---|---|
| Sequence 272 | cMhvSD070c02a1 |

CCGCGGTGGCGGCCCGAGGTACCAAAAAGACTCTCAAAAACCAATACTCCCACGGGCAAGGGAAT
AGCCAAGTTTGTTGCGGTTTCCAATGAATGACATCAGCCCTGTGTAGGTCTCAATCAAAATGGGTT
CAGTTAACACCATCAGTTTCTTTCCTCTTCCAGATCCAGTTGAATTCTTGTGGGCATTCTGGATAGC
TGGAACAAGCTTAGACATGAACCCAGACAACTTGCAAATTTCAAGGAATTTCTCACTGGTGTATTT
CATAGGATGCTCAGTGAAAGTAGCATAAGGAACTTCAGTGGACCATGGGTTCCGGCGGGACAGAA
GAGACTGCTCCTCCGGACTCCCCCAGTAGATCCTAAGGCCTTCTCCTTGTCTCTTGTCCAGGGACAT
CCCAGGGAAGGTGAACTTGCCCAGGCAGATGCGATAGACAGCGCTCAGAGGAATCCGCTGCAGCT
GCACACAACTCAGC

| | |
|---|---|
| Sequence 273 | cMhvSD070c11a1 |

AGGTACTTTCTCTTTGTCTCTGCCTTCCAGGCAACAGGGATTTTGGGGTAGTAGTTAGCTCTACAAA
TTATCTTGAGCAGTTAAAAGCCTTTGCAAGCTCAAAATTTACTGCTCTGGGCTCCTTCTGGGAAAA
GCAGTGGAAACTGCCCAATGCTGTAGCTTAGCAGTTAAGGCTTTGTCTTTTCACAATGGTGGCCTG
AGTTCAGGTTCAATTTTTAGCCTAGGAAAATGAGCACTTTCTGGTTGGCATTTGGGTGACCTGTGC
CATTTTGTTGGATTCTTCCTCCCCTTTCATAAACTGTCTTAAATTTTCCTTTTCTTCTGAGCACCTGG
GAGGNTACATTTTGGAAAAGTTAAAAAGCCAGGGAACCCGCGTACCTGCCCGGGCGGGCCGCTCT
AAGAACTAGTGGGATNCCCCCGGGCTGGCAGGAANTTCGATATCAAAGCTTATCGATACCCGGCG
ANCTCGAGGGGGGGG

| | |
|---|---|
| Sequence 274 | cMhvSD070g09a1 |

ACCGCGGTGGCGGCCGCCCGGGCAGGTACGCGTTTTACAAAGAGCAGCTTGTTAAGGCCAAAGAA
CAGTATTGAAAATTACAAGAAAACAGACCAGTAAATGGTCTGGGGAAGGATCATGAAATCCTGAG
GAGGAGGATTGAAAATGGAGCTAAAGAGCTCTGGTTTTTCCTACAGAGTGAATTGAAGAAATTAA
AGAACTTANAAGGAAATGAACTCCAAAGACATGCAAGATGAATTTCTTTTGGGATTTTAGGACAT
CATGANAAGGTCTATTAATGGACCGGATCTATACTTACCTCAGTTCATGACAGGATTGGNAAGCCA
GNGTTGAATTTGGGCCGGGGAAAAAAAGGAGGCCCAAAAAGTATCCTTGAACAAGGAAACTTGG
GTTCCAGGCCGNGAGGAAATTAACCATTAATTCNTTTCAAGAAAATCCCCAAAGGGGGACCTTGG
CAATCANAAAGGCCNAAAAAAAAGCC

| | |
|---|---|
| Sequence 275 | cMhvSD071c10a1 |

CCGGGCAGGTACCCAGGGAACAAATGCTACTGGGGCTCCACACCTACCTAAGAAGCAGCTCTACC
CAGACTCCACATGGCTCTCTGTTTTGGTCTGGAGACCCCAACTGGGGTATCTCCTGAGCCCAGGGA
TTCAAAGGTTCGTGGCAGAAATATGCATCCCACGGGACTCTCACTCACTCACCATTTTCTTGTAGG
GGGATTCCCCTGGGTCTGTGCCACTCCTGGGTGAATGGTTGATCTGTCTCACTCTTCTCCGTGATCC
GAAGGTCACACTATGTCACTGATGAATCCTTATGTGTCCACCTGGATGTTCCGGTTGAAGAGCTAG
TGTCTCACCACTCTTTCTGCTATTTGTGAGAGTGGCACACACTAGCTGCTTCTAGTCAACCATCTTG
GCCCCACCTCACTCACTTTTCTCAAGTAATCAAAGACCAGAAAGGATGTCCTTTACAAGAACAGAT
CCCCCAAAATGTAAGAGTTCACTGAAAAGGNGGGAGCTCAAACCAAGAGAGGACTTATCTCGCAA
CATAAAGACAACTTGTACCTTGGGCCGGTCTAGAACTAAGGGGATCCCCGGGCTGNAAGGAATTC
NATATNAAAGCNTATTGGATCCCNCNGACCTCGANGGGGGGGCCCGGGA

| | |
|---|---|
| Sequence 276 | cMhvSD072d05a1 |

CGGGGGCCATTGAGACTGCCATGGAAGACTTGAAAGGTCACGTAGCTGAGACTTCTGGAGAGACC
ATTCAAGGCTTCTGGCTCTTGACAAAGATAGACCACTGGAACAATGAGAAGGAGAGAATTCTACT
GGTCACAGACAAGACTCTCTTGATCTGCAAATACGACTTCATCATGCTGAGTTGTGTGCAGCTGCA
GCGGATTCCTCTGAGCGCTGTCTATCGCATCTGCCTGGGCAAGTTCACCTTCCCTGGGATGTCCCTG
GACAAGAGACAAGGAGAAGGCCTTAGGATCTACTGGGGGAGTCCGGAGGAGCAGTCTCTTCTGTC
CCGCTGGAACCCATGGTCCACTGAAGTTCCTTATGCTACTTTCACTGAGCATCCTATGAAATACAC
CAGTGAGAAATTCCTTGAAATTTGCAAGTTGTCTGGGTTCATGTCTAAGCTTGGTCCAACTATTCCA
GAATGCCCACAAGAATT

| | |
|---|---|
| Sequence 277 | cMhvSD074f04a1 |

CCGCGGTGGCGGCCCGAGGTACTGAGCGCGCGAGGCTCTACAGAGTGAAGGTTTAAATCCAAGGT
CATGGCAAAACATCTGAAGTTCATCGCCAGGACTGTGATGGTACGCGGGGGACTCGGGGTCGCCT
TTGGAGCAGAGAGGAGGCAATGGCCACCATGGAGAACAAGGTGATCTGCGCCCTGGTCCTGGTGT
CCATGCTGGCCCTCGGCACCCTGGCCGAGGCCCAGACAGAGACGTGTACCTGCCCG

| | |
|---|---|
| Sequence 278 | cMhvSD075c08a1 |

CCGCGGTGGCGGCCCGAGGTACGCGGGCCTGCTGCTGCTGCAGCCCCAGCTAAGGTTGAAGCCAA
GGAAGAGTCGGAGGAGTCGGACGAGGATATGGGATTTGGTCTCTTTGACTAATCACCAAAAAGCA
ACCAACTTAGCCAGTTTTATTTGCAAAACAAGGAAATAAAGGCTTACTTCTTTAAAAAAAAAAAA
AAAAAAAAAAAAAAAAAAAAAAAGGTNCATGGTCATTTGAAAGGCAAAATCTTTATTTACTTACT
TATTATTTTATTTTTTGTAGAGATGAGGCCTCACTATATTGTTCAGGCTGATCTTGAACTCTTGGGC
TCAAGTGATCCTCCTGCCTCAACCTCCCAAGTGCTGGGGTCATAGGCATGAGCCACTGTGCCTGGC
CCAGAATCCTTTTTAAAATGATGATGAAATGCCAGAGTCTTAGATACTCAGCACTCACTATCCAGG
CCATTTTGCCGGGTAGAT

| | |
|---|---|
| Sequence 279 | cMhvSD075c10a1 |

CGAGGTACTTTNTTTTTTTTTTTTTTCTTTTTTTTGAGACGGGATCTAGCCCTGCAGCCTCTGCCTCC
CAGGCTCAAGCTATTCTCGTGTCTTGGCCTCCCGAGTAGCTGGGATTACTGGTGCATGCCACATGC
CTGGCTAATTTCTGTATTTTTAGTAGAGACAGAGTTTCACCATGTTGGCCAGGTTGGTCTCGAATTC
CTGGCCTCAGGTGATCCTCCCACCTCAGCCTCCCAAAATGCTGGGTTACAGGCCCGAGTCACAGGG
CCTGGCCTAGCCCTATCTTTACCATTAGCTCCATTTTACAAGTTGTCATGGGGGGTAGTACACAGA
AGGATCGCGCAGCTAAAAAGCAACAGGGTTGGGAGTGGAAACCAGGTTTGTGTCCTCCTCTCTTCT
TCGGCTCCCTAGTCGCCTTGGGGAGTTCCCACCAATGGGGCCCAAACCTGATCATCAAAATCAACA
GGAAACATCTTCAAAAAGGGTCCAGGGCCCGCC

| | |
|---|---|
| Sequence 280 | cMhvSD075g12a1 |

CCGCGGTGGCGGCCGCCCGGGCAGGTACGCGGGGTAACTTTTTAACTTTATAAACTTAGTATTTTA
ACTTTTTAAACTTTTTTGTTGAAAACTAAGACACAAAAACACATGTTAGCCTAGATCCACACAGGG
TCAGGGTCATCAGTATCACTGTCTTCCACCTCCACATTTTGTCTCTGGAAGGTCTTCAGGGGCAATA
ACACACATGGAGCTGTCATCGCCTGTGGTAACAACGCAGAGTACCT

| | |
|---|---|
| Sequence 281 | cMhvSD075h03a1 |

CGAATTGGAGCTCCACCCGCGGTGGCGGCCGCCCGGGCAGGTACGCGGGCTCCTACTTGGATAAC
TGTGGTAATTCTAGAGCTAATACATGCCGACGGGCGCTGACCCCCTTCGCGGGGGGGATGCAGTG
CATTTATCAGATCAAAACCAACCCGGTCAGCCCCTCTCCGGCCCCGGCCGCTCTAGAACTAT

| | |
|---|---|
| Sequence 282 | cMhvSD076e12a1 |

GGACCGAGGGTTTGGTGCACCTCGATTTGGAGGAAGTAGGGCAGGGCCCTTATCTGGAAAGAAGT
TTGGAAACCCTGGGGAGAAATTAGTTAAAAAGAAGTGGAATCTTGATGAGCTGCCTAAATTTGAG
AAGAATTTTTATCAAGAGCACCCTGATTTGGCTAGGCGCACAGCACAAGAGGTGGAAACATACAG
AAGAAGCAAGGAAATTACAGTTAGAGGTCACAACTGCCCGAAGCCAGTTCTAAACAATTATTTTT
ACTAAAATGCATAATTATGTGATAGTTATACATATACCAACCTGTTATGTGAGACAAGCTGACCTG
CAAGTAGTCCAAGGCCAGTGAATCAATTACTGCTTGTACCT

| | |
|---|---|
| Sequence 283 | cMhvSD076f12a1 |

CGCCCGGGCAGGTACAAGCAGTAATTGATTCACTGGCCTTGGACTACTTGCAGGTCAGCTTGTCTC
ACATAACAGGTTGGTATATGTATAACTATCACATAATTATGCATTTTAGTAAAAATAATTGTTTAG
AACTGGCTTCGGGCAGTTGTGACCTCTAACTGTAATTTCCTTGCTTCTTCTGTATGTTTCCACCTCTT
GTGCTGTGCGCCTAGCCAAATCAGGGTGCTCTTGATAAAAATTCTTCTCAAATTTAGGCAGCTCAT
CAAGATTCCACTTCTTTTTAACTAATTTCTCCCCAGGGTTTCCAAACTTCTTTCCAGATAAGGGCCC
TGCCCTACTTCCTCCAAATCGAGGTGCACCAAACCCTCGGTCC

| | |
|---|---|
| Sequence 284 | cMhvSD076g02a1 |

CCGCGGTGGCGGCCGAGGACGCGGGCAAGCCCAAGGTTAAAAAGGCGGGCGGAACCAAACCTAA
GAAGCCAGTTGGGGCAGCCAAGAAGCCCAAGAAGGCGGCTGGCGGCGCAACTCCGAAGAAGAGC
GCTAAGAAAACACCGAAGAAAGCGAAGAAGCCGGCCGAGGTACCAATAGCAGGAGCAGAAAGGC
CAAAATCATGAGCGCAATTGCTGCGGGTCCCAGGCCCACATAGGAGTCATGCTGTGCTTCCCTGCA
GCCGCTGCCATGCAGACACTCACAAACTGTGAGTGTAAGGACCTGCTTTTCAGGACAACTAAAAC
CCTGA

| | |
|---|---|
| Sequence 285 | cMhvSD077a05a1 |

CCGGGCAGGTACGCGGGGTAACTTTTTAACTTTATAAACTTAGTATTTTAACTTTTTAAACTTTTTT
GTTGAAAACTAAGACACAAAAACACATGTTAGCCTANATCCACACAGGGTCAGGGTCATCAGTAT
CACTGTCTTCCACCTCCACATTTTGTCTNTGGAAGGTCTTCAGGGGCAATAACACACATGGAGCTG
TCATCGCCTGTGGTAACAACGCAGAGTACCT

| | |
|---|---|
| Sequence 286 | cMhvSD077g04a1 |

CCGCGGTGGCGGCCGCCCGGGCAGGTACGCGGGGAGAGGGAGCTGGGCAGGGCACAGCAGGGCA
GGAGTGTGTTTGATGTGTCCTGGGAACCGCCCTGAGGCCGTCGTGTGGCTGGAGTGCTGCANGTGT
CAAGGAAATTGTAGGAGATGTCTCCTGAGTGTGATGGAATATAACCAGATTTCCAGAAGGAACTG
ACATGATCTGACTTAAAAAGGCCACCTACATTTACATGAAGGCCGCCTACCTCAGCATGTTTGGGA
AGGAGGACCACAAGCCGTTCGGGGACGACGAAGTGGAATTATTTCGAGCTGTGCCAGGCCTGAAG
CTCAAGATTGCTGGGAAATCTCTACCCACAGAGAAGTTTGCCATCCGGAAAGTCCCGGCGCTACTT
CTCTTNCAACCCTATCTCGCTGCAGTGCCTGCTCTGGAAATGATGTACCTCGGGCGCT

| | |
|---|---|
| Sequence 287 | cMhvSD077g04a1 |

TCAACTTTATTGATANCCGTCNAACTTNGANGGGGGGGNCCCGGTCCCAACTTTTG

| | |
|---|---|
| Sequence 288 | cMhvSD078b12a1 |

CGAGGTACCCAGGGAACAAATGCTACTGGGACTCCACACCTACCTAAGAAGCAGCTCTACCCAGA
CTCCACATGGCTCTCTGTTTTGGTCTGGAGACCCCAGCTGGGGTATCTCCTGAGCCCAGGGATTCA
AAGGTTCGTGGCAGAAATATGCATCCCACGGGACTCTCACTCACTCACCATTTTCTTGTAGGGGGA
TTCCCCTGGGTCTGTGCCACTCCTGGGTGAATGGCTGATCTGTCTCACTCTTCTCCGTGATCCGAAG
GTCACACTATGTCACTGATGAATCCTTATGTGTCCACCTGGATGTTCCNGTTGAAGAGCTAGTGTCT
CACCACTCTTTCTGCTATTTGTGAGAAGTGGCACACACTAGCTGCTTCTAGTCAACCATCTTGGCCC
CACCTNACTCCCTTTTCTCAAGTAATCAAAGACCAGAAAGGATGTCCTTTACAAAGAGCAGATCC

| | |
|---|---|
| Sequence 289 | cMhvSD079b04a1 |

CCGCGGTGGCGGCCGAGGTACCGCGGGATAGTAACTTCTTATGGAATTGATTTGCATTGAACACAA
ACTGTAAATAAAAAGAAATGGCTGAAAGAGAAAAAAAAAAAAAAAAAAAAANGTCCT

| | |
|---|---|
| Sequence 290 | cMhvSD079h02a1 |

GTGGCNGCCCGGGACCGAGGGTTCGGTGCACCTCGATTTGGAGGAAGTAGGGCAGGGCCCTTATC
TGGAAAGAAGTNTGGAAACCCTGGGGAGAAATTAGTTAAAAAGAAGTGGAATCTTGATGAGCTGC
TAAATTTGAGAAGAATTTTTATCAAGAGCACCCTGATTTGGCTAGGCGCACAGCACAAGAGGTGG
AAACATACAGAAGAAGCAAGGAAATTACAGTTAGAGGTCACAACTGCCCGAAGCCAGTTCTAAAC
AATTATTTTTACTAAAATGCATAATTATGTGATAGTTATACATATACCAACCTGTTATGTGAGACA
AGCTGACCTGCAACGTAGTNCAAGGCCAAGNGAATCAATTACTGCTTGTACCTCGGCCGCTCTAGA
ACTA

| | |
|---|---|
| Sequence 291 | cMhvSD080g12a1 |

AGGTACCATATTAAGTGGAGAGCTGCAGCAAGGTGGCCCCTACAGCCCGCAAACCAGCCTGCACA
TTACCTCTCCATACTGCAGCCCTTTATATGGAAACTTNTTACATCACTTTGCTGTGTGTGTTACACA
AGGTGGGGTTTTGCTGTACCTGCCCCGNACCGGCCNTTTCTAGAACTAGTTGGATCCCCGGNCCTG
NAGGAAT

| | |
|---|---|
| Sequence 292 | cMhvSD080g12a1 |

AGCTGTTTCCTGGTGTGAAAATTGGTATTNNGCTTCACAATTCCACACAACAATACNAANCCCGGG
AGCCATAAAAGTGTA

| | |
|---|---|
| Sequence 293 | cMhvSD082b03a1 |

CCGGGCAGGTACTTTTTTTTTTTTTTTTTTTTTTTTTTCTGAGGNCAGCGTATGTGTATTTGGTGGGG
AAAACCTAATTTCGGGGATTTCTGTGGTAGGTAATAGGANAANAAAGGGCACTGGGGGCTGTTCT
CCTTCCTTCCCTGGGCTGTATCCATGGACTCCTGTGGCTGTCAGGCAGGGGGATTGTGATGGGAGC
AGCTTTCCTGGAGTCCTTCACAGNGGCGTTTACCTTCATAGTTGATACAACCATTGCTGTCCTCATG
CCCTGCCACCAGCATCTNTACTTCTTCCTCTGTCATCTTCTCACCCAGTGTGACAAGAACATGCCGG
ATTTCAGCACCCATGACGGNGCCATTTCC

| | |
|---|---|
| Sequence 294 | cMhvSD083f02a1 |

AGGTACTCATTTAACAGGCCGTGATTTTTCTCCCGCCCCCTTTGTTGTTCCAAAAGAGTGATTTATA
TGGAAGTTTACACTAGTGCCAAATACCACTGTAGTTAAAATGAGACCAGTATCATGGCCTAATTCT
AACGTCCCAGCAGCTTTGAACAATCATGATTTATTTTCTTAAATCAAATTTCAACTCAAGCTGCTTG
ACAGAAGCTTGTCAATACATGTGCTGTATTTTTTTTGCATTTGTTGAAAAATTGCACATATAGAATT
CCAAACATTTCTCCTGGTAGGTTCAGTTACACAAATACATGTTCTATAGAACACTGAGAGGTTACT
TTTGAGTTAAGTCCACAAATCTTCCATAAGTTCAACCTAATCAGTTACCAGTTCAAGAAGATCTTG
AAGGTGGTAAACTAGCAGGAACTTCAGATTTAGGAAACCCGCGTACCTGCCCG

| | |
|---|---|
| Sequence 295 | cMhvSD085d10a1 |

AGGTACAAGCAGTAATTGATTCACTGGCCTTGGACTACTTGCAGGTCAGCTTGCCTCACATAACAG
GTTGGTATATGTATAACTATCACATAATTATGCATTTTAGTAAAAATAATTGTTTAGAACTGGCTTC
GGGCAGTTGTGACCTCTAACTGTAATTTCCTTGCTTCTTCTGTATGTTTCCACCTCTTGTGCTGTGCG
CCTAGCCAAATCAGGGTGCTCTTGATAAAAATTCTTCTCAAATTTAGGCAGCTCATCAAGATTCCA
CTTCTTTTTAACTAATTTCTCCCCAGGGTTTCCAAACTTCTTTCCAGATAAGGGCCCTGCCCTACTTC
CTCCAAATCGAGGTGCACCAAACCCTCGGTCC

| | |
|---|---|
| Sequence 296 | cMhvSD085e11a1 |

AGGTACTGTCCAACTGGATGCTGCCCTGGTGGCTGAAGGCACACTTCATGATGCTGTCCAGGGTCA
TCAGGGAGACATGTTGAAAGAGCTCCAGACGTGAGTTTTGGGCAATGTGTTCCTCCCATTTGTTCA
GCATCATCCGAACACTCTCAGACATCATGGTGATGAATATTTTCAGAATGCTGATGTTGAAGCCAG
GTTTCACAATCTGGCGGTGCTTTTTCCATTTAGAACCATCCAGGGTCACAAGTCCTCGACCAACCC
AGGATTCAAGGATTTTGTGGCTAACAGCACTTTTGGGATCTTGTCTTTTCAGGAGAATCTTGGCAT
AGTCTGGGTCATGGACACTGAAGAACATCGTAAAGGGTCCAACCCACAAGGGAACAGCACATGGG
TATTTTTCCATCAGCTTATGATACACCTCAAACTCCTTTACTGGGTAAAACTCCTTGTGGCCATAGA
ACCAGTGGGCAG

| | |
|---|---|
| Sequence 297 | cMhvSD085f07a1 |

AGGTACAAGTTGTCTTTATGCTGCGAGATAAGTCCTCTCTTGGTTTGAGCTCCCACCTTTTCAGTGA
ACTCTTACATTTTGGGGGATCTGCTCTTGTAAAGGACATCCTTTCTGGTCTTTGATTACTTGAGAAA
AGTGAGTGAGGTGGGGCCAAGATGGTTGACTAGAAGCAGCTAGTGTGTGCCACTCTCACAAATAG
CAGAAAGAGTGGTGAGACACTAGCTCTTCAACCGGAACATCCAGGTGGACACATAAGGATTCATC
AGTGACATAGTGTGACCTTCGGATCACGGAGAAGAGTGAGACAGATCAACCATTCACCCAGGAGT
GGCACAGACCCAGGGGAATCCCCCTACAAGAAAATGGTGAGTGAGTGAGAGTCCCGTGGGATGCA
TATTTCTGCCACGAACCTTTGAATCCCTGGGCTCAGGAGATACCCCAGCTGGGGTCTCCAGACCAA
AACAGAGAGCCATGTGGAGTCTGGGTAGAGCTGCTTCTTAGGTAGGTGTGGAGTCCCAGTAGCAT
TTGTTCCCTGGG

| | |
|---|---|
| Sequence 298 | cMhvSD086c05a1 |

TAGGGCGAATTGGAGCTCCCCGCGGTGGCGGCCGCCCGGGCAGGTACGCGGGGTAACTTTTTAAC
TTTATAAACTTAGTATTTTAACTTTTTAAACTTTTTTGTTGAAAACTAAGACACAAAAACACATGTT
AGCCTAGATCCACACAGGGTCAGGGTCATCAGTATCACTGTCTTCCACCTCCACATTTTGTCTCTGG
AAGGTCTTCAGGGGCAATAACACACATGGAGCTGTCATCGCCTGTGGTAACAACGCAGAGTACCT

| | |
|---|---|
| Sequence 299 | cMhvSD086h11a1 |

CCGGGCAGGTACTGTCCAACTGGATGCTGCCCTGGTGGCTGAAGGCACACTTCATGATGCTGTCCA
GGGTCATCAGGGAGACATGTTGAAAGAGCTCCAGACGTGAGTTTTGGGCAATGTGTTCCTCCCATT
TGTTCAGCATCATCCGAACACTCTCAGACATCATGGTGATGAATATTTTCAGAATGCTGATGTTGA
AGCCAGGTTTCACAATCTGGCGGTGCTTTTTCCATTTAGAACCATCCAGGGTCACAAGTCCTCGAC
CAACCCAGGATTCAAGGATTTTGTGGCTAACAGCACTTTTTGGGATCTTGTCTTTTCAGGAGAATCT
CGGCATAGTCTGGGTCATGGACACTGAAGAACATNGTAAAGGGCCAACCCACAAGGGAACAGNA
CATGGGTATTTTTTCCATCAGCTTATGATACACCTCAAACTCCTTT

| | |
|---|---|
| Sequence 300 | cMhvSD087e02a1 |

ATAGGGCGAATTGGAGCTCCCCGCGGTGGCGGCCGAGGTACAATGTTGTCTTTATGCTGCGAGATA
AGTCCTCTCTTGGTTTGAGCTCCCACCTTTTCAGTGAACTCTTACATTTTGGGGGATCTGCTCTTGT
AAAGGACATCCTTTCTGGTCTTTGATTACTTGAGAAAAGTGAGTGAGGTGGGGCCAAGATGGTTGA
CTAGAAGCAGCTAGTGTGTGCCACTCTCACAAATAGCAGAAAGAGTGGTGAGACACTAGCTCTTC
AACCGGAACATCCAGGTGGACACATAAGGATTCATCAGTGACATAGTGTGACCTTCGGATCACGG
AGAAGAGTGAGACAGATCAGCCATTCACCCAGGAGTGGCACAGACCCAGGGGAATCCCCCTACAA
GAAAATGGTGAGTGAGTGAGAGTCCCNTGGGATGCATATTTCTGCCACGAACCTTTGAATCCCTGG
GCTCAGGAGATACCCCAGCTGGGGTCTCCAGACCAAAACAGAGACCATGTGGAGTNTGGGTAGAC
CTGCTTCTTAAGTTAGGTGTGGAATCCCAGTNNGCCATTTTGTTCCCCTTGGGTACCTGGCCCCGGG
GCGGCCGTTTNTTANAACTTAGTNGGAATCCCCCCCGGCNTGCAAGGAATTTTCNAATATANAAGC
CTTTATTNGATACCCGGTCGAANCTNGAAGGGGGGGGG

| | |
|---|---|
| Sequence 301 | cMhvSD088b12a1 |

AGGTACACGTCTCTGTCTGGGCCTCGGCCAGGGTGCCGAGGGCCAGCATGGACACCAGGACCAGG
GCGCAGATCACCTTGTTCTCCATGGGGGCCATTGCCTCCTCTCTGCTCCAAAGGCGACCCCGAGTC
AGGGATCCCCGCGTACCTGCCCG

| | |
|---|---|
| Sequence 302 | cMhvSD088b12a1 |

GTTAATTGCNCGCTTGGCCGTTAATCAATGGGTCATAAGCTTGTTTTCCTGTGGTGGAAATTGTTAT
CCCGCTCACAAATTTCTCACACCAACNATAACCGAAGGCCGGGGGAGCAATAAAAGTNGTAAAAG
CCCCTGGGGGGNGCCCTTAAATGGAGGTGGAAGCTTAAACCTCAACATTTAAAATTTGNCGGTTN
GCGGCCTTCAACTTGCCCCCGCTTTNTNNCAATTCCGNGGNAAAACCCTTGTTCCGATGGCCCCAG
CCTGGCCAANTTAAAATGNNAAATNNGGCCCAAACNGCCGCCGGNNGNAGGAAGGGCCGGGTTT
TTGCCGGTAATTTGGGGCCGCCTCCTTTNCCGGCTTTTCCCTTCGGTTTCACCTGGACTTCNTNTTG
CGGCTTCGGGTCCCGTTTCCGGCTTG

| | |
|---|---|
| Sequence 303 | cMhvSD088c07a1 |

AGGTACACTCTTCCTTAAGTCCAGTGGTGCAGGAAAGCTTCAGTTTGTCAATATCACGCAAGACAG
GGACACCAAACACTACCCCTGCCCAAAGGAGCCCCTCACGGACGCCGCCATGTTGTTACCGGACC
CGAGCACCGCTCCCCGCGTACCTGCCCG

| | |
|---|---|
| Sequence 304 | cMhvSD088c12a1 |

AGGTACGCGGGGACGGTTCGTTTTTCCTTTANTCANGAAGGACGTTGGTGTTGAGGTTAGCATACG
TATCAAGGACAGTAACTACCATGGCTNCCGAAGTTTTGCCAAAACCTCGGATGCGTGGCCTTCTGG
CCANGCCGTNTGCGAAATCATANTGGCTGTAGTATCCGNTGCTATCCCTGGGGGTTGCAGCTTTGT
ATAAGTTTCGTGTCGGCTGATCAAAGAAAGAAGGCAATACGCANATTTCTACATGAAACTACGAT
GNTCATGAAAGCATTTTGAGCGAGATGANNGAAGNGCTGGGTATCTNTTCAGGAGTGTAAAGGTA
ATCTTNGGGAAATATAAAA

| | |
|---|---|
| Sequence 305 | cMhvSD088f07a1 |

AGGTACAGTGGCCCCCCGTGAAAGACAGAATTGTGGTTTTCCTGGTGTCACGCCCTCCCAGTGTGC
AAATAAGGGCTGCTGTTTCGACGACACCGTTCGTGGGGTCCCCTGGTGCTTCTATCCTAATACCAT
CGACGTCCCTCCAGAAGAGGAGTGTGAATTTTAGACACTTCTGCAGGGATCTGCCTGCATCCTGAC
GCGGTGCCGTCCCCAGCACGGTGATTAGTCCCAGAGCTCGGCTGCCACCTCCACCGGACACCTCAG
ACACGCTTCTGCAGCTGTGCCTCGGCTCACAACACAGAATTGACTGCTCTGGACTTTGAACTACCT
CAAAATTGGCCTTAAAAATTAAAAAGAAGATCGATATTAAAAAAAATTANNAAAACNNNATGAA
AAAAGNGTCCCTTGCCCNGGGCCGGCCCGTTNTTANGAACTAGTGGGATCCCCCGGGNCTGCAGG
GAAATTCCGATNTTCAAACTTNATTNGAATACCCGNCTACCTANAAGGNGGGGGGCCCCGGNTNC
CCAAGCCTTTTTT

| | |
|---|---|
| Sequence 306 | cMhvSD088g11a1 |

GGANATGGGGTTTTGCTGTGTTGCCCAGGCTGGTCTNTAACTCCTGGGCTCAAGCAATCCTCCAGC
CTCGGCCTCCCAAAGTGCTGGGATTACAGGCGTGAGCCACCGCACCCGGCCACTTGTTTCTTAATG
AGTGTCTGCAACTGCTGGGGAGGTGCGGGTCTGCCGGCCAGAGCTGCAGGTAAGTGAGGGTCAAG
CTGGTTCACANAGTGCANCAACTCAGCTNANAGTCCTGAACACACAGCCCAGCCCTTTGAAACCA
TCCCCTCCAGCACAAGGAAGACAGCATTNTGCAAACNCATCCATGGGAGCCTCAGGAAAATAAGT
TTTANACAAGTCACGTGTTCCTACCTTCCAGGCANCAAAGTCAGTGNTACAGAAAGCAAAGTANG
GGGATCGCAGGCCTCTGGCTGGAGGGAGGCCNCCAAAACTCCCTGGGATTAGNATTTCGGNTGAC
TCTAANGCCATCAGGGGTTTANCTCNACACCTAAAAGNCTACTCTGNNGGATTCNAAANCANACA
GTTACCTTGNCCGGGGCGGGCCGGGTTTAAAAANTAAGTGGNATCCCCCCGGGGCCTTGGGAGGG
AAATTTCCAATATTNAAAGCNTTTTTCANATACCCGTCAACCCTCGAGGGGGGGGGGCCCCGGGN
ACCCCCAANCTTTT

| | |
|---|---|
| Sequence 307 | cMhvSD088g12a1 |

AGGTACAAAGTGGGAGCTGGCACTGGGCAGATCTGGCTGGATAATGTTCAGTGTCGGGGCACGGA
GAGTACCCGGAGCACGGAGATCTCGCCGGCTTTACGTTCACCTCGGTGTCTGCAGCACCCTCCGCT
TCCTCTCCTAGGCGACGAGACCCAGTGGCTAGAAGTTCACCATGTCTATTCTCAAGATCCATGCCA
GGGAGATCTTTGACTCTCGCGGGAATCCCACTGTTGAGGTTGATCTCTTCACCTCAAAAGGTCTCTT
CAGAGCTGCTGTGCCCAGTGGTGCTTCAACTGGTATCTATGGGGCCCTAGAGCTCCGGGACAATGA
TAAGACTCGCTATATGGGGAAGGGTGTCTCAAAGGCTGTTGAGCACATCAATAAAACTATTGCGC
CTGCCCTGGTTAGCAAGAAACTGAACGTCACAGAACAAGAGAAGATTGACAAACTGATGATCGAG
ATGGATGGAACAGAAAATAAATNTAAGTTTGGTGCGAACCGCCATTCTGGGGGTGTCCCTTGCCG
CCTGCAAAGCTGGTGCCGTTNGAGAAGGGGGTCCCCCTGTACCCTGCCCGGGGCGGCCGCTCTAA
GAACTAGGTGGGATCCCCCGGGCCTGGCAAGGGAATTTCGATATCAAAGCCTTTNTCGGATACCC
GGGCGNCCCTCGGAGGGGGGGGGCCCGGGNACCCCCANCTTTTTGG

| | |
|---|---|
| Sequence 308 | cMhvSD090e01a1 |

AGGTACTGAGCGCGCGAGGCTCTACAGAGTGAAGGTTTAAATCCAAGGTCATGGCAAAACATCTG
AAGTTCATCGCCAGGACTGTGATGGTACGCGGGGGACTCGGGGTCGCCTTTGGAGCAGAGAGGAG
GCAATGGCCACCATGGAGAACAAGGTGATCTGCGCCCTGGTCCTGGTGTCCATGCTGGCCCTCGGC
ACCCTGGCCGAGGCCCAGACAGAGACGTGTACCTGCCCG

| | |
|---|---|
| Sequence 309 | cMhvSD090e10a1 |

AGTGGAAAAGGCTATTGCCCACTATGAACAGCAGATGGGCCAGAAGGTGCAGCTGCCCACGGAAA
CCCTCCAGGAGCTGCTGGACCTGCACAGGGACAGTGAGAGAGAGGCCATTGAAGTCTTCATGAAG
AACTCTTTCAAGGATGTGGACCAAATGTTCCAGAGGAAATTAGGGGCCCAGTTGGAAGCAAGGCG
AGATGACTTTTGTAAGCAGAATTCCAAAGCATCATCAGATTGTTGCATGGCTTTACTTCAGGATAT
ATTTGGCCCTTTAGAAGAGGATGTCAAGCAGGGAACATTTTCTAAACCAGGAGGTTACCGTCTCTT
TACTCAGAAGCTGCAGGAGCTGAAGAATAAGTACCTGCCCGGGCGGCCGAGGTACCGAGCATGAA
CATCTGCAGCCTCTTGCAGAATCACCCCAGAAGGGGACTGAATCATGGTCCTCTTGATAGGTATGT
TCAGCAGAGTTTCCAGTCCTGAGGTGTATGAGGCCAGCTGGAGCTCATAATCCTTAATTGAATTGG
CGCAAAGTTCAGCAATTTTTTGTCCTGCCCG

| | |
|---|---|
| Sequence 310 | cMhvSD090f09a1 |

AGTGGAAAAGGCTATTGCCCACTATGAACAGCAGATGGGCCAGAAGGTGCAGCTGCCCACGGAAA
CCCTCCAGGAGCTGCTGGACCTGCACAGGGACAGTGAGAGAGAGGCCATTGAAGTCTTCATGAAG
AACTCTTTCAAGGATGTGGACCAAATGTTCCAGAGGAAATTAGGGGCCCAGTTGGAAGCAAGGCG
AGATGACTTTTGTAAGCAGAATTCCAAAGCATCATCAGATTGTTGCATGGCTTTACTTCAGGATAT
ATTTGGCCCTTTAGAAGAGGATGTCAAGCAGGGAACATTTTCTAAACCAGGAGGTTACCGTCTCTT
TACTCAGAAGCTGCAGGAGCTGAAGAATAAGTACCTGCCCGGGCGGCCGAGGTACCGAGCATGAA
CATCTGCAGCCTCTTGCAGAATCACCCCAGAAGGGGACTGAATCATGGTCCTCTTGATAGGTATGT
TCAAGCAGAGTTTCCAGTCCTGAGGTGTATGAGGCCAGCTGGAGCTCATAATCCTTAATTGAATTG
GCGCAAAGTTCANCAATTTTTTGTACCTGCCCGGGCGGCCGCTTCTANAACTAGTGGATCCCCCCG
GCTTGCAGGGAATTCGANATNAAGCTTATNGATACCGTNNACTTTAGGGGGG

| | |
|---|---|
| Sequence 311 | cMhvSD090f12a1 |

AGGTACCAGCAGACCCCAGGCCAGTCTCCACGCACACTCATTTTCAGCACAAACACTCGCTCTTCT
GGGGTCCCTGATCGCTTCTCTGGCTCCATCCTTGGGAACAAAGCTGCCCTCACCATCACGGGGGCC
CGGGCAGATGATGAATCTGAGTATTACTGTGCGCTGTATATGGGTAGTGGCATTTGGGTGTTCGGC
GGAGGGACCAAGCTGACCGTCCTAGGTCAGCCCAAGGCTGCCCCCTCGGTCACTCTGTTCCCGCCC
TCCTCTGAGGAGCTTCAAGCCAACAAGGCCACACTGGTGTGTCTCATAAGTGACTTCTACCCGGGA
GCCGTGACAAGTGGCCTGGAAGGCAGATAGCAGCCCCGTCAAGGCGGGAGTGGAGACCACCACA
CCCTCCAAACAAAAGCAACAACAAGTACCTGCCCGGGCGGCCGCTCGACCCGGGCAGGTACGCGG
GGGGGCAAAAAAATCAAGGTATTTGGTCCCGGAACAAAGCTTATCATTACAGATAAACAACTTGA
TGCAAGATGTTTCCCCCAACCCACTATTTTTCTTTCCTTTCAATTGCTGAAAACAAAAGCTCCANGA
AGGCTGGGAACATACCTTTTGTCTTTCTTTGGAGAAAATTTTTTCCCCTTGATGTTTATTTAAGNAT
ACATTTGGGCAAAGAAAAAGGAAAGAGCCAACCACGGATTCTTGGGGATCCCAAGG

| | |
|---|---|
| Sequence 312 | cMhvSD091a07a1 |

GCATTGAATCAACCTCAGCCACCATCTGCTTTTAACAGCCAGGAGAAACCAGTAGTAGCCAGCAG
ATCGCGCCTACCAACCAGTTTCACCAACTAGCAGGTAACTCCGGGTTTCCAATCTGTCCATCCAGG
GAGGAAGAAATGCAGGAAATGAAAGATGCATGCACGATGGTATACTCCTCAGCCATCAAACTTCT
GGACAGCAGGTCACTTCCAGCAAGGTGGAGAAAGCCAATCACACATCAAGAGATGAAGACACTG
CAGTACCT

| | |
|---|---|
| Sequence 313 | cMhvSD093b03a1 |

CCGGGCAGGTACGCGGGGTAACTTTTTAACTTTATAAACTTAGTATTTTAACTTTTTAAACTTTTTT
GTTGAAAACTAAGACACAAAAACACATGTTAGCCTAGATCCACACAGGGTCAGGGTCATCAGTAT
CACTGTCTTCCACCTCCACATTTTGTCTCTGGAAGGTCTTCAGGGGCAATAACACACATGGAGCTG
TCATCGCCTGTGGTAACAACGCAGAGTACCT

| | |
|---|---|
| Sequence 314 | cMhvSD093d07a1 |

AGGTACGCGGGGACACCAAACAACTCATTACACAAAGAGGTAAGGTCCCAGACCACGCCAAAGCT
TCCTGAGACCTCTCCTCATCTGTGCATGGACGGATGACCAACTCTGGGGCCCAGGCTGTTGCTTCC
CAGTATAATGATGAATCCGCCATAGTCTGGTGAGTGTAGAGGCTGACTCTGGAGCCCAAGCTGTAC
CTGCCCG

| | |
|---|---|
| Sequence 315 | cMhvSD094b01a1 |

CCGGGCAGGTACCACTCTTTACCAAACTGCTAAAGGAATCGAAACCTTCTCCAGAGGTCAAATGGT
CAGGAATTCGAGTAAGGCACACTCTCAAATCCTTAGTAAGGCCAAATATCTTTTTAAATTCAGCAT
CACTCTTCAGATGTAATGCCTTATTACTTCTTCCTTGAGAATCTTTCTTGTCATTTCTTCCTTGGGCA
CCTTTATCTGATGTTGATGCCATCTCATTATGGATCTTTGGTTCTTGGTCTCTAAAAACACACTGTT
GTGAAGTAACAGTTGTTGCATCAACAGAGGAACTTATTTTCTCTATGCTCTGGAGGACACTTNCTT
TTCTAGCATAAATGACGGCTGTTACTTTTCTGGGGGCATTGTGTNCTGTACCT

| | |
|---|---|
| Sequence 316 | cMhvSD094d05a1 |

CCGGGCAGGTACCCAGGGAACAAATGCTACTGGGACTCCACACCTACCTAAGAAGCAGCTCTACC
CAGACTCCACATGGCTCTCTGTTTTGGTCTGGAGACCCCAGCTGGGGTATCTCCTGAGCCCAGGGA
TTCAAAGGTTCGTGGCAGAAATATGCATCCCACGGGACTCTCACTCACTCACCATTTTCTTGTAGG
GGGATTCCCCTGGGTCTGTGCCACTCCTGGGTGAATGGCTGATCTGTCTCACTCTTCTCCGTAATCC
AAAGGTCACACTATGTCACTGATGAATCCTTATGTGTCCACCTGGATGTTCCGGTTGAAGAGCTAG
TGTCTCACCACTCTTTCTGCTATTTGTGAGAGTGGCACACACTAAGCTGCTTNTAGTCAACCATCTT
GGCCCCACCTCACTCCTTTTNTTCAAGTAATCAAAGACCAGAAAGGATGTCCTTTTACAAAGGAGC
AGATCCCCCCAAAATGTTAAGAANTTCACTTGAAAAAGGTGGGGAAGCTCAAACCAAAGAGAGG
GACTTTATCTTCGCAAGCCATTAAAGACAACCTTTGTACCTTCGGGCCGCTCTAAGAACTANGTGG
GATNCCCCCGGGGCCTGCAGGGAATTCGATTATCAAANCTTTATCGGAATACCCGGNCGAACCTTC
NAAGGGGGG

| | |
|---|---|
| Sequence 317 | cMhvSD094e07a1 |

CCGGGCAGGTACCCATGGGAGATGGACTGGCTTGTTCTTTGGGTCAACTGCAGCTTATTGGAGGTG
TTGATATGGCACTTAGGGTCTTTGCTCCCTTGATATATCTTCTGAGGGTAGCAAGGGCAATTCTACT
GCAGAGGCANTGGCAGAAAGGATTTCATTTGCTCCTGGAAGCTCTGTCCAAAAAACTGCTGAGTT
GCTACTGGCTTGATAGCTCCGGTGGTGGGCTGGCTAGAGACCCAGGCCAGGAGGACCTGCCCATC
AAGTAGAGTCCGGTCAATTTTCTGTAGGGCTGCTGTGGTATGCTGGGGGGTCCCTCCANTCCCCTA
ATTGCCTCATATTTTTTCCCAGGGGAAGAATGATAGCNCTGCCCCCTTTTCTNTTGGGAAGCTNTTG
TNCCTTCNGGNCCGNCCCGGGCCAGGGTTACTTTTTTTTTTANTTTGACNAGGAGGGAACAATGCC
CTTTTAAAAAAATATTTTTAATTGGGGTNGAAAACTTTTCTTAATTCTCAAGGAAAACCTTTTGGGN
TNCTTTTAATATAAATTTAATTNATGCTCTTTAAAAATTTCTGTTTGGATNNAAAAGCANTTGGTAT
TATTATTAAAATACCCTGTTAAAAGAAAAAATANTANTTTTTAAAAAT

| | |
|---|---|
| Sequence 318 | cMhvSD095c03a2 |

CCGGGCAGGTACGCGGGGTAACTTTTTAACTTTATAAACTTAGTATTTTAACTTTTTAAACTTTTTT
GTTGAAAACTAAGACACAAAAACACATGTTAGCCTAGATCCACACAGGGTCAGGGTCATCAGTAT
CACTGTCTTCCACCTCCACATTTTGTCTCTGGAAGGTCTTCAGGGGCAATAACACACATGGAGCTG
TCATCGCCTGTGGTAACAACGCAGAGTACCT

| | |
|---|---|
| Sequence 319 | cMhvSD095c05a2 |

CCGCGGTGGCGGCCGAGGTACAAGCCTTGAACATCGTCCTGCTTCCCAGTGGGTTCAGACCTCACC
TCTCAGGGAGCGACCTGGGCAAAGACAGAGAAGCTCCCAGAAGGAGAGATTGATCCATGTCTGTT
TGTAGGACGGAGAAACCGCTTGGGTAACTTGTTCAAGATATGATGCATGTTGCTTTCTAAGAAAGC
CCTGTATTTTGTGATTGCCTTTTTTTTTTTTAAGATGCTTTCATTTTGCCAAAATAAAACAGATAATG
TGGATGGTTTAAGGGTTATAGTATTATAGTTTAAATAA

| | |
|---|---|
| Sequence 320 | cMhvSD095c09a2 |

AGGTACGCGGGTAAACTTGGCATTTCCAAAGGAGTAATGCCCCCATCTTGTATGTAACTCCAACTC
AAAGGAACAAAAGAGAGGGCCAATTTTATATGAAGTTTTATTCTCAAAATATAAAAAAAAAAACA
AAAACCCCACACACCAAGGGACTAAGATGATGTTATTTCACAGCACTTGCTTGCCTCAGTCTTTAC
GAAGAACACAATTCCAAACTAATGGACAAGTTCCTCCCTGTGCTCTAGGTCATTCAAAGGAGGCA
AGCTCCTTTTGTCAAATCAGGAGCTCCATCAGCTGATCAGGAGCCCAGATNCCAGGGTGGATTTTT
CTCAGTGGGATCTAGTATTGCTAGAAGAGCCTTCCTTACATGGCAAGAAACAGGCACATGGGCCT
NTTTCCTTTAGAATGCATCTTGTCTNACATGCTTTGGGGACTGCTTGNGCCANGAACCACCTTGGTG
TTGGCCTGGCNAAGGCANCNTNTTACATGGGCCCCCCCAAAAACNTGGGNCNTGGCNATTTTTTTT
TCCCGGCTTTTTTNNCANGCCCCCCTTNANGGNANNAAGCNCCCATTGCCACTTGGTGGGGCTTGG
GGTANTTTTNCCGGGAATTCNNNTTNNTTTCTCCCCGCAAAANAAAAANANTCNNNGGAAANTNC
GGGTTTTTTTTTNAGGGGGAAAA

| | |
|---|---|
| Sequence 321 | cMhvSD095d05a2 |

CCGGGCAGGTACGCGGGGTAACTTTTTAACTTTATAAACTTAGTATTTTAACTTTTTAAACTTTTTT
GTTGAAAACTAAGACACAAAAACACATGTTAGCCTAGATCCACACAGGGTCAGGGTCATCAGTAT
CACTGTCTTCCACCTCCACATTTTGTCTCTGGAAGGTCTTCAGGGGCAATAACACACATGGAGCTG
TCATCGCCTGTGGTAACAACGCAGAGTACCT

| | |
|---|---|
| Sequence 322 | cMhvSD095f12a2 |

CCCATAATGGCTATTTATTGGATCAGCAATTTATAAGTCCCACATTCTCATGCCACATAGCTNTACA
CAGNTGCAAAAATATACCATAGNTTGCAGGGGATCATTGGTTTGATAAAAGATATTGAGTCGCTC
ATTTTGTGAAAGNGACCTTTGATATAAGAGGAGCATNACGCGGGGAAAGCTCACATGTCCCGTGG
NTCACACACCAGAAGGTATTTGCGNNTTGTCATTGCTGTCTGGNAGGCCATGGCAATGGCTTTTTT

| | |
|---|---|
| Sequence 323 | cMhvSD095g02a2 |

CCACACACAGGACACACACAAATGCATGCCCCATGATCGCACTCAGGAAAAAACCCACGGNCTNC
CATATGGCTGNNAACAAACTNTAGTTTNTACCANTCCTGATGGTGAGCACGANTATGTNGAAAGA
AGCAGGCACAGCANAAGAGTTCGTTGTGCTCGNGGTCATGTAAATGTTGTATCTGGTGAAGGTGG
GTCATTGTTACATGACTGAATTGNNTCCCTTCAAAATTCATAGGCTGAAGCCCTAGTNACCGTTTTT
GNANACAGGGTNTTTTAGGAGGTTATTNAGGCTAAATGAANTCTTAAGGGGGGGCCC

| | |
|---|---|
| Sequence 324 | cMhvSD095h02a2 |

CCGGGCAGGTACCCAGGGAACAAATGCTACTGGGACTCCACACCTACCTAAGAAGCAGCTCTACC
CAGACTCCACATGGCTCTCTGTTTTGGTCTGGAGACCCCAGCTGGGGTATCTCCTGAGCCCAGGGA
TTCAAAGGTTCGTGGCAGAAATATGCATCCCACGGGACTCTCACTCACTCACCATTTTCTTGTAGG
GGGATTCCCCTGGGTCTGTGCCACTCCTGGGTGAATGGCTGATCTGTCTCACTCTTCTCCGTGATCC
GAAGGTCACACTATGTCACTGATGAATCCTTATGTGTCCACCTGGATGTTCCGGTTGAAGAGCTAA
GTGTCTCACCACTTCTTTCTGCTATTTGTGAGAGNGGGCACACACTAGCTTGCTTCTTAGTCAACCA
TCTTGGGCCCCACCTCACCTTAANTTTTNTTCAAGTNATTCAAAAGACCCAAAAAANGGNTGTCCC
TTTTACAAANAAGCCAGAATCCCCCCAAAAAATGTAAAGAAGTTCACTGGAAAAAANGGTGGGGA
AGCCTTCAAACCCAAGGAGAANGGACCTTTNTTNTTTNCCAGCATTAAAANGACCNACTTTGNGN
CCTCCGGGGCCGNCTTCTTANAAACTTANGTGGGAATCCCCCCCGGGCCTTGGAAGGGNAATTTCN
GANNNTTCCAAGCCTTTANTCGAATNCCCGGCCGNACCCNTGAGGGGGGGGGGC

| | |
|---|---|
| Sequence 325 | cMhvSD003a01 |

AGGTACTGANANAAAAATNTGCTCTGTGGGNNNAGCNTATCCAGTCCACAGCCCCTNTCTTGGTN
ATTNATAAAGACAANGATCTGCNCTNAGGGATNCCTNAGCNATTCTCCAATCTCCATCTCACGGTA
CNACAATCACCTTGACCATCAGNGG

| | |
|---|---|
| Sequence 326 | cMhvSD004c08 |

CCGGGCAGGTACCACTTTTATCACATGCAGCTGCCTTAACCAACAGGTTTTCTAAGATACTATCCC
CCTTACCTGTTTCTGCCTCTTTCAATGGTGTTTTTCCATTTTTACAGACTTCTGAAAATTTTAGCTTT
CATTGAAATAAGCTTCCCCATTCCTTCATGTTAATATATCTAGCAATATTGAATAGAAATTATAAAT
GGAAATAAAAATGCTTGCTTTTATAAAATCTCCAGTCTCGCAGCACCCCCAATATAATACAAACAG
ACTTAAGTTGAAATTTGGTTTGTTAATGCCCACCTTGTGTGGTCAAAACACAGTTTTGAAGGAATG
ACCACCTTCAATGTTCTTTACAGCTTCTTTAGTGTTACTTAAAAAAAAAAAATCAATCTGATGGAT
GATTGATGGTANGTTTGTTCATGGAAGATCTTCATCTTATGGGAATTATCTAGTTTTTCTAATCATA
TACTACCAACAAAAATAAACACAAGCGTGTTCCCTTTAATCATATTATCCTCCACCATTACTTCCA
AAAG

| | |
|---|---|
| Sequence 327 | cMhvSD007c03 |

CCGCGGTGGCGGCCGNCNGGCCANGTNCNACTAANATCTTCANTNNACTANCANGATAAACAGGN
CNATNAATAACTGAGGNNAAGCCCNANTNGCAAGGNCACACANGAAAGAATCAGACCACGAAAT
GAGCTNCNNNTGNCACCTGCANNGGGNGCACNATGAGGNTTTNTGAACTCNATGAGCTACCGAGC
CACGGNTTCTCGATGTAGCACTCTTATTAGTGTCGCCCTGCGGCGCCGGTCTACAAGCGACGNGGT
CTGTTTTATCCATTATACCACAGGGGAAGGGACCGNTTNAGTGCTNCGAAGGTTATACNCAGTACT
GTAATCCACAGGCACAAGACCACCTACTCATTGNGCATNCNCCAAGCTCTCNTGGNCCAGAACAC
CTTCTNAGNATGCTATGNGGGCATTNCTNGCGCNCAAGCTCGGTANGGGAAATAAANATNTATTA
TTNGGCCTTTANTCCAATTACCCTGGCCTTAATCCCTCTGNGGGGGGGG

| | |
|---|---|
| Sequence 328 | cMhvSD009a02 |

CCGCGGTGGCGGCCCGAGGTACAAAGTGATCAAACCTGTCTATTAATTAAGCAAATGAGTGGTGA
ATCACTGAGACGGCTGGATGGCTGAGCTGAGGGATGTGATGTGTGCCCAACGTCCTGCAGGGTGC
TGGTGAATAACATGAGAAAGAACTTAAAATGGCTTGATGATCTCACCATTTAGTGACCTTGGTTGT
CACACTGCTTTCCAAGAGCCCTTTAAAGGTAGGAATGAGAGCTGTTTCCAGTATGCATTCCAATAG
GAATGCAGCTTTGCTAAAGTTAGAGACATAAACTAAAACCCTGTGAAGTCCTATAGAGCCCTTGG
ACTTATTTCCTAGCAAGCATTTATCATCCCCACCATCCTCTACTTCAGGACACCCGCGTACCTGCCC
GGGCGGCCGCTCTAGAACTA

| | |
|---|---|
| Sequence 329 | cMhvSD014h08 |

TCCTATAGGGCGAATTGGAGCTCCCCGCGGTGGCGGCCGCCCGGGCAGGTACTAATCATCCTGTCC
CAACAACCATCCAATCCACACCCCATCTACTCCCACAACTTTTGTAAGCAAATAACAGCCCAACGT
TTTATCCACAAATGTTTCCGTATGTATTTCTAAAAGATAAGGCCTTTTTCTTAAACTACCCACATCG
TCACACTCGAAAAAAAGTAGTGACTGCTTGATATTAGATATTCAATTACGTTAAAATTTCCAATTA
TCTCACAAATGCCGCACATTTAAAAATTTTTTTTATTCAATCACAAATCATGTCCATATTATAGAAC
ATTGGGATTTGAACTCAGGCCTGCTTCCAAAACTTGTATACTGCCAACTTTGTCATGCTATAAGAA
TGCATGCATGGAGAGAGACAAGACAGAAATAAAGCCTTTCTTGTCCTTTAAATGTCCTGCTCTGCA
GTAGGAATTGTAAGGTAGGTAAGTAAATAGATGTNCTGAANGCTACCTCTGACCTTTTAAAATCTT
TGACATAGATAGGTTGAGAAGGCAGCAATATACCTTTAACCAAACTAACTACCAAAGGAAATTTG
GAAANGGGCACCAGA

| | |
|---|---|
| Sequence 330 | cMhvSD016c06 |

AGGTACTGCCTGCCTCTAGTGTCGCGTCCCTCCAGTATCCGATGGGAGCGCCGTCCGCAGGGAATG
TGTCTCTCTGATCATGGTGTCTCGTGTCCAACTCTGGGGGAAGACCGAGACAAATCGAGTCACTGG
TGNTGGGAAAAGGCTTATTTCCGCTTNCGCTTGNCCANTTTCANGAATTTGATTCTGAGAGCNGGG
CTNCNGTTNCANGCNNGGNTTGTACCTNCCCG

| | |
|---|---|
| Sequence 331 | cMhvSD026a03 |

CGGCGGCCGCCCGGGCAGGTTNACATGGTNCGGCTTNAATACTCCCAGTTNNTGANNCNGCNCAC
AAGCCCTGNGANCNNGGCNANNTNCCNATATNCNGAGACTGACAGGGCTTANTAAGAACCNNCC
CATCNGACATNNGANGGAGANNAAGGNGCNGNACNAGNCCGCNGAAANAANCATACCCTGAGAA
TNCCNNNCNACCAANAGGNATTTGAGCNGCCTGTTTGATGTAAGAAAAGGA

| | |
|---|---|
| Sequence 332 | cMhvSD027e05 |

TATCGGCGAATTGTAGCTCCCCGCGGTGGCGGCCGNCCNGCCATGTANGCTNGATANCCTNCAAC
CCAGAAAGATNTANTTNCGCGAGCACNNCTNNNGCCANNTAGCNAGACATTTTNACCCGAATGCC
GTNANNTTNAGGAATNCCCTNNTNCNGANTNTTTTGCTTCNTNCCACCCCTANGGGGAAANACTGC
TTTGTGCTTTGG

| | |
|---|---|
| Sequence 333 | cMhvSD035a01 |

GCNCCACTGCACTCCAGCCTGNGTGACNGATCAAGACTCTGTCTTAAAAAAAGAAANAAAATAAN
GTGAATATCAGTATTGCTTGAAAATTCCTAGAATATTTGGATAAAACTTTAAATGAANACATGAAT
AACTGACTTTGGGAACTGTAATTGTACCAAATTTTGTTTTTCCAAAAACAANAAAGTAACCTTGGT
TCCCAATACAACCAGAATTTTGATATTCCTTGNACTGCATGCCT

| | |
|---|---|
| Sequence 334 | cMhvSD036g08 |

CTGTCTCACTGACTGNGGATGAGGATGGGAGGTCAGCTACTCACTGGTTTTCACTGACATTANGGG
TATANGGAACCANAGTGCTGACTAGCCCTGACTNGCTCTACTGTATTCAATCTCATTGNTGNCAGG
TNTATATGGGGNGTGAGTNTATCATAACACNNACTANCACTACCTNACACTACCA

| | |
|---|---|
| Sequence 335 | cMhvSD037f08 |

AGGTACCCGGGNACCTGATNCATTTCTACCNNNCTNTAGNAGAANCACATCTTANTGGTGNNATN
CGTCTGTTCTTNCTCACGNATGCCGCCCCNACNAGGCNTGACAGACCATACTAGGCCATANGCANC
GACTTGT

| | |
|---|---|
| Sequence 336 | cMhvSD045f05 |

TGGAGCTCCCCGCGGTGGCGGCCGCCCGGGCAGGTACTTNCTTTTTTTTTTTTTTTTTTTTTAAAAN
NCCNNNNAAANNGGGGATNCCCCGGGGNANANCCNNNGNCNNANNNGAGNAANAAGGNGGTAA
NNAAAAAAGGCTCCCTGAATNAANNNNNTTTNGCCCTATNANGGNGGTTTTTTATTGCCCCNNGG
CNNGAATATNCCNCNCNNAAANGGCCCCCCGCTTTTTTTTTTTTTTT

| | |
|---|---|
| Sequence 337 | cMhvSD045g01 |

ACGTACCAGGATNTACANTNNAACCATCTTTTCCGGNNAGNCCNCAAGNANNAGCTGNGCCCCTA
NGANNANAAAGACCNACGGANCCNGGGGCANNTTGATNACNATGGNNACCANCCCNNGNGTACN
TGNCNGNNCNGACGTTTTAAAACTANAGGNTTCNNCNNTNTGAAGGAATTGGATNTCANNNTTNT
TGANANCGTTNACTTCTAAGGGNGGNNCNNNNCCNACTTNTNNTTCCCTTTAGNAATGNTTAANN
GCANNCTTNNNNNAATAATNNTCATNCTTNTNAACTGGGTCANGANATTTTGCCGTATGAACATCA
CAGAGTGTACCT

| | |
|---|---|
| Sequence 338 | cMhvSD046e03 |

AATTGGAGCTCCCCGCGGTGGCGGCCGAGGTACAGTGGCCCCCCGTGAAAGACAGAATTGTGGTT
TTCCTGGTGTCACGCCCTCCCANTGTGCAAATAAGGGCTGCTGTTTCGACNACACCGTTCGTGGGG
TCCCCTGGTGCTTCTATCCTAATACCATCGACGTCCCTCCAGAANAGGAGTGTGAATTTTANACAC
TTCTGCAGGGATCTGCCTGCATCCTGACGCGGTGCCGTCCCCAGCACGATGATTAGTCCCAGAGCT
CGGCTGCCACCTCCACCGGACACCTCAGACACGCTTCTGCAACTGTGCCTNGGNTACAACACANAT
TGACTGNTCTGACTNTGACTACTNAAAATTGGCCTAAAAATTAAAAGAGATCNATCTAAAAAAAA
AAAAAAAAAAAAAAANTTCCTNCCCCGGNCGNCCNNGNAAAAANCCGGGTTTTTTTATTCCCCTN
AANNGGAAATGAAAAAATTTNGCCTTTNNCNTCCNAATTTGGNCNNTTTATTTNCCNNNNGAACTT
TNTTTAAANNGNNACTTTTTTCCNNTTTNAAAAAAANGGGTTGGGGGNNCCCCCCGGCCATTTTNN
CNGCCANTTCCCNTTTNGAGAAAANAAAAAATTTTTTTTTTTCCCCNNNGAAACAAANCCCTTAAA
AAAAAT

| | |
|---|---|
| Sequence 339 | cMhvSD048b05 |

TGGAGCTCCCCGCGGTGGCGGCCGCCCGGGCAGGTACTTTCTTTTTTTTTTTTTTTTTTTTTTTAANA
NCCGCAGNTCNNTNTTATNCCTNCNNAAAAAAAANNNNTNTNCCTNTNGCCATTNTTTAAAAAAA
CNNTNACNTNTTNTTNNAAAAANANNTTTNNTTTAAAAAANNTNGNNCNAAATNNNNTTTNNGGG
GNAAAAAAAAAAAANNNTTTTTGNNNNCTNNTTTTTTAAAAAAAANNTTTTTTTTTNTNACCCAAA
NGNNGGCGTNTTTANTNTNNCCCCCNTTCNNAATGNNATTTTNAAAAANAGNTATCCCCCGNNNC
NGNNGANNNTNANNNAAAATTTTTNNANCCCCCCCCCCN

| | |
|---|---|
| Sequence 340 | cMhvSD048d04 |

ATCTNCATTAGGGCTATCATTCCTATCCANATTCCCACAGGCTCACAGNTAAGCTACTNCAACAGC
TGTTGCTGACTAAATATNCTCATGTNTCTAAATAATTATNTAAATANGGAACAGNGGATTNATACC
TGATNCCTCTACATTAAAAAATATTTCTTTCATTATTACATCAANAGTAAAATATATAAAACATTCT
GCCTCAATTTCAAGGTCTTNATTAAGTTGGTACCT

| | |
|---|---|
| Sequence 341 | cMhvSD048e12 |

AGGTACTTTATTTTTTTTTTTTTTTTTTTTCNNTTTTAAAAAAAAAAGGGGGNNNTTTTTTTAAAAAA
AANNGGGNNCNNTTNCCAAAAAANNTTNNTGNNTNCCCCCCCCNTTTTCNAANGGGNATTTTNNN
NNNNGGGNNNCCCCCANGNTTTTTTTNTTNGNATTNNNAANNTNGTNTTCCCCCATTNNTTTTTTTT
TTTANNNCCCCCTTTTNAAAAAANNNNNNNGNGAANCCNTTTTNNGCCCNNNNNAAAAATTTNAA
NNTTTTTAANCCCCTTAAAAAANNCCCCNTTTTTNNGGNGNCCNCCTCCCNNTTNNATTTTNAANA
TTTTTTTTTTNAAGGGGGGNGGATTTTTTTNNANNNNNNTTNNNCCCCNANNGNCCTTAAANNNNN
NTNNNNTNCCCCCCCCNNTCCCNGGGGGNTTTTTTTCAAAAAANTNTTTTTTTTTNANCCNTTTTTG
GGNCCCCGCCCCCCCNNNTNANCCNTTTTTTTTTTTTTTTAAAANTGGNCAAAAANTNACACTNNN
TTTTTTTTNCCAANANANCNATTTGGGGNAACCNCCCCGGGGGCNTAAAGCCCCGGGGGGGNTTT
NNGGCCCCCCNCCCNGGGTTTTTTTTTNGGGGGGCCCNNNTCTNTTTNAAAAANCCAAAAAAANTT
TTTT

| | |
|---|---|
| Sequence 342 | cMhvSD049c01 |

ACGAGGTACCGCGGGTCAGGAAGGTGAGGGCGAGACCCCTACCCCCACAGAGAGCAGCAGCCAT
GGGGAAGGGCAAAACCCCAAAACNCTANTGGAAGAAAAGCCCTATCTGTGCCCCGAGTGTGGAG
CCNGCTTCACAGAAGTTCGCAAGNCCCTACTNTTTCNNATAGGGAAGCNTTGNCCACCCCCAGGGT
TGNTCTNCCCTNGNGNAAAAATGGGNGTTCTTGGTAGAAACTCAAGGAGGGCNCCTTCTGCTCTTT
NCTCTCCNGGAAGTAGNNGAAAACCAACTTGGGAATTTTTTTTNTGNCCCCNNCAAANAANAANA
AATNNTNTCNNCNGGGGGGNGNANAANGGGGGGANGGGANTTATANCCCCCCTTATTCNANANA
ATTGGGTTANGGCTNGGGNGANGNTTGNGGANGTGGAAAGAATANAAGTANACCCCNCTNGNGN
GAAAAAAAAAATANTTAGGTTNGTCNTTTTTTTACNNTACNANGNTTGTAATTGTAAGGTAAAAA
NCCCCCTTATTTAAAGAAAATTTGGTCTTGGGCTGGGNGGNANAGNCTACCTTTAATTAAANGGGC
CAGTTTNTTAGGAAAAAAAACCTGTGTTGGGTGTTTTAAGAAAAA

| | |
|---|---|
| Sequence 343 | cMhvSD056d03 |

AGGTACTTTTTTTTTTTTTTTTTTTTTTTTNGGAAGGTTCTCAGGTCTTTATTTGCTCTCTCAAATTCC
AGGAATTGACTTATTTAATTAATCCATCAACCTCTCATAGCAAATATTTGAGAAAACAAATTGATA
TTCAGATTCTTATTTTCAGCAGGGAAGTAAGAAGTTGCAGCTCAGTGCACATAAAGTTTGAGACAG
AGATGGAGACATCCAGCCCCACCTNTCTGGAACAAGAAAGATGACTGGGGAGGAAACACAGGTC
ANCATGGGAACAGGGGTCACAGTGGACACAAGGTTGGGCTGTCTCCCCACCTCCTCACATTAGGC
TTACAGGGACGCAGACACATTCAGGTGCCTTTGCANAAAGAGATGCCAGANGCTCTTGAAAGTCA
CAAAGGGGAGGCGTGAAGAAATCCTGCATCTCAGTNCCTTCACAAAGACAACTTGGTTTANGCTTT
TNAAGCTTGTGAGGAGACACACCCNGCGTTACCCTGCCCCGGGCCGGCCGCTTTTAAAAACTAGTG
GGNTCCCCCCGGGCTGCAAGGAATTTCGATNTNAAACTTTATTGATTCCGGNCNACCNTTGANGGG
GGGGGCCCGGGTACCCCAACTTTTTGT

| | |
|---|---|
| Sequence 344 | cMhvSD058h02 |

AATTGGAGCTCCCCGCGGTGGGCGGCCCGAGGTACTTTTTTTTTTTTTTTTTTTTTTNNCCCNNCNTT
TCCCGGNNAAAAANNNTTGANTTCNNNNTANNNAAANANNACGTTNTTCANNGGGGGAAAAAAA
GGCCNCANNNGGGNGGGNGNNNACNATGNNACCCNNGGGNNTTTNNGGAAGANGGGNGCTCAA
NNACAAANCCNTNNAANNNNGGGGNNNTTTTGNNNCCCNAANCNGGGGCNAAAATTGACNCCCN
CNCGGCNGNNGGACTTNCNTTNGGNNAAAAAAAGTTNNANTNTTNNNATACAANTTANAANTTNA
ANGGGTAATAANNGGNTNNNCNNGCCAAANTGAAGACATAAATACATATNCTGTNGGGCAAANC
NTTTTCACCCGNCCTAAGANAACATGCCCCCCCNCAAAANCAATCCCCNAACNTTTCCCNAANCA
AANGGGGGAGCCCNTTAATCCTGTTTTTAACATACNNGCTCANTGACGNGGGTACTAAGGATAGA
NTCCNCCNCCATTGGGTTTGAGCCATAACTGGANTCCCAAAAGGCTTTGGGGTACCNNACCATTTT
TTNAGGGAGGAGGGGANAAATTGNGTGAATTTACCCCATGCCAAAGCTTAANANGGGCCTCGNCT
AAANCCCACNGGCGCCAATNTNCAAAATCNTGGGTTTCCANCCTCACCTNGGAAATGCCCCCCCA
TTGGGAGGANGGGGGACNTTNGGAAGANGGACCANGGGGGGATTCTGGAANTANCCCCATGCTTT
NAACAAAGCTNAACTTTTNTCCTTT

| | |
|---|---|
| Sequence 345 | cMhvSD059c01 |

CCGGGCAGGTACAGTGGCCCCCCGTGAAAGACAGAATTGTGGTTTTCCTGGTGTCACGCCCTCCCA
GTGTGCAAATAAGGGCTGCTGTTTCGACGACACCGTTCGTGGGGTCCCCTGGTGCTTCTATCCTAA
TACCATTGACGTCCCTCCAGAAGAGGAGTGTGAATTTTAGACACTTCTGCAGTGGATCTGCCTGCA
TCCTGACGCGGNTGCCCGTCCCCCAAGCACCGGTTGAATTAAGTTNCCAGGANCTCGNGCTTGCGC
AACCTANCAACCCGGGAACTNCCTNNANGAACAACGCCTTTTCTGCCAAGCNTGTGGCCCTTCGG
GCTTTCAACAAAACCAACNAGTANTTTGGACNTTGGCTTTCNTGGAACNTATTTGGAACCTTAACC
TTCCATAATAAATTTTGGGGNCCCTTA

| | |
|---|---|
| Sequence 346 | cMhvSD060d09 |

AGGGCNAATTGGAGCTCCCCGCGGTGGCGGCCGAGGTACGCGGGNNACAGANNTNTTNCNANCA
GTTTCTACAAGGCNTGAATCATNGNNNTAAGAANATTGCGANGGGATTACTNACAANAAATTNNN
GTTGACCATCTCNGCAGACACTGGTGTGNGGCGGGAAATTNACCTTTGTTTTTTNCTAGCCNCGGC
TNGNNGNGCTNAATCNNCACCTTNGCCCNNGGNTGCTCNTNCNTNCNNNCCGNNACCNCTGGAGG
NAAANNGTNNCNTATTCTCAGCNANTTCTGCATGCTCTCCNNAGCCTNCTGCANATTCTAACAAGG
GGGGCGCNNGATNCACAATGCCTCTTCCAANCACGAGNGGGTNTTCTTGGGCTCAAAATATATTTG
TTGGATCCANNNNCNGNNATCCTTTTCCAACACATTCCCACCTATTGTGGGAACAGATGGCATTAT
AAGAACATTGTGTTTGATGAAAATC

| | |
|---|---|
| Sequence 347 | cMhvSD060d10 |

NATTGGAGCTCCCCGCGGTGGCGGCCGCCCGGGCAGGTACGGATNCNNCNTGNCCNANGNTGGNN
NAANGGTATCNTNCTGNTTGAACNNCAATTCAGATNATAATGAGGAGNATTNGGCCTNGGAGAAA
CTAAACTGATGGNCTTAATGGGCTAAATNCCNATGNTNAATCCTTATGGATTTTNGGNGCGNTGGG
ATTGTNTGTTGAACTTATTATAAGANAAANGGGCTTCCAAAGTGCGACCACNTACTGTGTTCCCGN
CCTGACAGNNCAATGGCCTAAGCTNNTTTGAAATNTATNAAANGNNCANTNTNTNNANTGNNGAG
CAATGGNTNCTTTCCAGACAGGAAGACTGCTGCTAAGTACCCTCGGC

| | |
|---|---|
| Sequence 348 | cMhvSD061b07 |

CGAGGTACATGTGNGCCCCCCGTGAAAGACAGAATTGTGGTTTTCCTGGTGTCACGCCCTCCCAGT
GTGCAAATAAGGGCTGCTGTTTCGACNACNCCGTTCGTGGGGTCCCCTGGTGCTTCTATCCTAATA
CCATCGACGTCCCTCCAGAAGAGGAGTGTGAATTTTAGACACTTCTGCAGGGATCTGCCTGCATCC
TGACGCGGTGCCGTCCCCAGCACGGTGATTAGTCCCAGAGCTCGGCTGCCACCTCCACCGGACACC
TCAGACACGCTTCTGCAGCTGTGCCTCGGCTCACAACACAGATTGACTGCTCTGACTTTGACTACT
CAAAATTGGCCTAAAAATTAAAAGAGATCGATATTAAAAAAAAAAAAAAANNANNAANNNCCTN
GCCGGGNNAAACCTTTTANATTNGGGNANCCCCNGGGNTNTNNGANNTNNAAAAAAANNNTTTTN
TTCCCNCCCCCNGGGGGGGGGGGCAAAAAAAAAAANTTTTGGNCCCTTTANNGNGGGNNTANTGG
NCCNTTTGCNNCCCCNGGGG

| | |
|---|---|
| Sequence 349 | cMhvSD061d01 |

AATTGGAGCTCCACCCGCGGTGGCGGCNCGAGGTACTTTNTTTTTTTTTTTTTTTTTTTTTTNNNNN
NCCCCCCCCNTTTTTTNAAAANNCCNTTAAANNGGGGGGGGGGNNAAAANCNNNTTTTTTTGGGN
NNNAANNNNGGGGGGGGGGNAAAANNCCCCNCTNNNNNGGNCCCCNTTTTACANTNGGTTNCCN
AANGNTTGAANNTTTNGGGNGNTTNANAAAACCCCNTTTTTNTNNTTTTTTTNNCNAAAAAAATNG
NNGAAAGGNCCANNGCNCNCANCNNCCANANNGNGAAANNCCCGNGGGNNAAANNNGCCCCNA
AAATGGGNCCCCANTTTTTCNCNCNNTNGGGGGGGNNNNAANANTANGGGCCCCCNTAATTTTGA
AANNTTTTTTNNNTCCCAAAANTTCGAGGTGAGNGGANTTTTTTNAAACCCCANCACCCCCNTTTT
AAAAAANNNGNNNTTNNAAAGGCNCNACAAANTTTTGGCNCCCNGAGGGGTCCNGTNNGNGNTN
TTCACNCNGGGGNCNCTTTAAAANATTTTTTTTGGGNNNNNCCNNNAAAAACGGGGTTACTANTN
NCCCCCCATAACCTCAACCTTTGGNANTNCAANTGTGCAATGGCTNGNCCTTGNACCCTNGGGGTT
TTTGCCCCTGNCCCNANNGGGCCCTGCCCTAAAAACCNCANNTTATNCCCCCCCCCTNTTTTAANG
GNNCNTCNATNAANGGNACNTTCTTTTNAAAAAATNNNANANNNNNNNNNNNNNNNNNNGNNCC
CCCCCCCCC

| | |
|---|---|
| Sequence 350 | cMhvSD061d05 |

CGCCCGGGCAGGTACAGTGGTGTGATCTCGGCTCACTGCAACCTCTGCCTCCCGGGTTCGAGTGAT
TCTCCTGCCTCAGCCTTCAGCTTGCACTACCACGCCCAGCTAATTTTTGTATTTTCAGTAGAGATGG
AGTTTCACCATGTTGGCAAAGATGGTCTCTATCTCTTGACCTTGTGATCCACCCGCCTTGGCCTCCC
AAAGTGCTAGGATTACAATATTGGATTTTATGTTAGCACCAGCCTGTCCTTTATTGATCATACCATT
TACCTGGACTCTTTTCTTCAAGAACACAATCTAAGNAATCCTAAACCAGTTTTGACACAAACCATT
GCCTTTAACAACCCATTCATAGTGAGGGGATTTANTGTAGTTTCAATGTCACCATCCAAGATCCCA
CCCCAGTACCTCGGCCGCCCCNGGCAGGTCCCNGGGACAAGGGCNACCCAGCTCTCAAANGAACT
GGNCCAGCTTCCGGATGCCTATTAAAAACAGAAGGAGCNGCTTGNGGNAACAACTAGAANCCCCT
TCCAAGCCAAAAGGAATGGGCNCTTTTTTCAGGAAAGCCGGGAACTTTTTGCCAAANTTNAAAATT
TTATTGGAAAAAACCCCCCGGAACCTGGAGGANGGGGTTTNAGCCTAATTTCTTGGCGGGTTCTTA
ANNAGGAAAAAAACTTGGGACCAAAGGNTTTTNGGNAAAACCCCGCTTGGNANTCCNGGNAAAT
AAAGGNGGTTTTNAAACCCCTGGAACNAAAGGCCCGGGANATTCCCCCTCCAAAAANGGAACCTG
GGGGACCAAATTTCTTTTGGAAGGAAAAAAAAA

| | |
|---|---|
| Sequence 351 | cMhvSD062d12 |

CCGGGCAGGTACTTTTTTTTTTTTTTTTTTTTTTTTTTTTAAAAAANGGGNTTTTTTTTTTCCCCCCNA
GGGGGGGGGGGGGGNNCANTTNNGNTNNNNNGGCNCNTNNNNCNNGGGGNNNAAANNANTTNC
CCNTNTTTNTCCTAAAANNNAAAAAANNCAGGNGTNCCCCCCNCCCCCNTTNNTTTNTAAAAAAN
NNNCNTTTTNAAAAAAGGGGNTTNTNTTNNTTNNCNNNANNNNTNAAAANCNNCNGCCCTAAAAN
NANTTTTNNGCNTNGCCCCCTAAAANNNNNTTTTTNTANGNNNAAANCNAGGGCCNNGGCNNAAA
AANAATTTTNNGCCANNAATNNGAAAAANCCTGNTNTTTTTNTTNAGAGGGGAAANTTTCAANCN
CNNCTTTTTTAAANAAAAAAAGNTTNGTGGGACANANNTGCCNTNAAAAAAAAACANGATATTTA
TGGGNAGATANTTNACCCCATNANNNCNCCNCTGGGGGGGGTTCATGAANACATCCCNCCCCCNN
TAAAAATAGAAAAAACCCCCCCCTGTCGNGAATTTNTTTTAAANTTTTTNNNNCCCCCCCCCCN

| | |
|---|---|
| Sequence 352 | cMhvSD063g04 |

TGGAGCTCCCCGCGGTGGCGGCCGCCCGGGCAGGTACTTTNTTTTTTTTTTTTTTTTTTTTTNTAATT
TTTTTTTTTTTTTTTTTTTTTTTTTTTTTTTTTTTAAAAAAANACNNNNNTTTTTTTNNNGACNCANTT
TTTNNNNAAAAAAAAAANACCCTCNNNTTTTTTTTTTAANGNCNNCNNNNNNTAAAAAAANTTTTT
TTTNNTNCCCNGGGGGGGGGNGCCAACNCNTTTTNAANAAATNCCCANGNGGNGGGGGANCCCN
ANACAATNATNNANGNANCNCCCNAAAAAATTTAAAAAAACCCCCCCNTTTTTGGGGANGANNCC
NNNNNTTTTTNTAAAAAANNCACCGGNCACCCCAAANANGNTTNTNTAAAAAAANCCCCNNTTTT
TTCANAAANGGGGGGGGGNGACNNAAAAAAAAAAAAATTTTTTTTTTTTTGNNGGGGGATCNTTT
TTCCNNGNTTNNAAAAAAAAAAAANCCCCCCCCCNNCGGGAAAAAATTNAAAAANNTTTTTTNNC
CCCCCCCCCCCCGGGGGGGGGCCCCCCCCCCNNTTTTTTTTTTNTTTTTAAANAANAAAAAAAACC
C

| | |
|---|---|
| Sequence 353 | cMhvSD074a08 |

CGANGTACGAGACCTGCTTCTATCTCCTGAAGAAAACTGTGGCNTTCTGGAATGGGAAGATAGGG
AACAAGGAATTTTTCGGGTGGNTAAATCGGAAGCCCTGGCAAAGATGTGGGGACAANGGAAGAA
AAATGACAGAATGACATATGAAAAGTTGAGCAGAGCCCTGAGGTANGTTAATAGCATANAATACT
ATGANCCTTCANGAAGAGTTATATACAATGGCTGGCTGTAGAAAATTACACTGTTTTTGCAGGTTT
TTTACTT

| | |
|---|---|
| Sequence 354 | cMhvSD083h08 |

TGTNTCCACACCTGTCCTNTTGGAGTTTGGATGGCAAAGACNTGCGAGGTGGTTTTGGGCACACCT
AANGTCTGTTTCAGGGGTCCTGAATGAGGTGATTGCNACNACTCAAAGACTAAGTTTNTAAGATCC
CAGGCATGGAGTAAAGCAATTCTATACACAGGATCTCAATCCTAGTCACAAAGACTTCTTAATGAT
ACATGGGCTCAAAGACATNGGTTCCCCTGAACACNTCAGCTTGGATTCATACTGNCCCCATATTTT
CCAGTGTGCCATGTAGTTATCCTTTATNACCCTCGTAACCATGCCCAT

| | |
|---|---|
| Sequence 355 | cMhvSD085d05 |

GNTNTCNGGNTTCCNTCTNNCTNAGNNNAAANNNCNCTTNATNCTGTTGANGCAAGAGNGACNGN
ACATNCANCCCTNNCNACCCAGNCTGNNTTTCACTGNANANCAAGGNTGAGGNAGCTTCAGGGCN
ACACTGCGAGTTTCTATGCATGAAATNNTCCTAGCATTTTGCGTTCTCATAACTANAATATGGCTTG
TGTTGCAAGACCAATGATACTGNGAACNNTANNTNCCCNGNCNGCCNNTCTAGAACNAGTGCGAT
NCCNNGGGCTGCNTGAATTGAGATNTCAATCTTATCCTTNCCGTACGACCTGGGAGGNGGGGCCC
GGCTACCCAGAATTTTGGTTCCCTTTTACNCGAAGGGTCTAATTGCGCTACTTAGGCCGTAAATCA
ATGNAACATGAGCATGNCTCTCCTGGTGGCGAAAAATTGGAGTATANCCGTATCATCAAATATNTC
ACCACGAACTNTACCGCATCACCTTGGAAGCCATTTATAGCAGTTNAAAGCACTANCGGGNTGCC
CTNAACTNGAAGTTGGAANCTTAAAACTTNACCAATTTNAATTTGGCCGTTTNGGGGCATTAAACC
CGCNCCCCCCCCCCCTACCCCCCGNGAANAAANNCTGNCCCCCCNTTNNCCCCCCTTTNATTTTAN
CTCCCCCCCCCCCCCCCCCC

| | |
|---|---|
| Sequence 356 | cMhvSD086h05 |

AGGTACTCATGGTCTGCCAACCCTGGCTTCACTTGGCACGGTTGATTTAGGTGCTCATGTCACCAA
ACAGCAGAGCCATCCTGAGCAGAATTCAGTAGACTATTGCCAACAACTGACTGTGTCTCAGGGGC
CAAGCCCTGAGCTCTGTGATCAAGCTATAGCCTTTTCTGATCCTTTGTCATACTTCACAGATTTATC
ATTTAGTGCTGCATTGAAAGAGGAACAAAGATTGGATGGCATGCTATTGGATGACACAATCTCTCC
ATTTGGAACAGATCCTCTGCTATCTGCCACTTCCCCTGCAGTTTCCAAAGAAAGCAGTAGGAGAAG
TAAGCTTTAGCTCAAATGATGGNGATGAATTATTAGAAATAAACAGACCCCAATTTATNAACTGG
GAAAGCAATTTTNTGCTTGGGNGCTATGCAAATTATGCNTCTGGGGTTTCAATATTGTTTGCTTTTG
GCTTTATTTTTTTTTTTTTTTAAAAGGGAATGTNGNTGGNTTCATTGGNAAAAAAACCTNGTTTTGG
AAAGCCCCACCCNAAAGNAATTTTCCCNNGGGAGGAAAAAAACCNTNANGTGGGTTAAANGGNA
AATTNTTTTGGGGGGGGCCAAAAAAAAAAAANGGGGGGTT

| | |
|---|---|
| Sequence 357 | cMhvSD087d02 |

CGCGTAATACGACTNACTATANGGTNTAANGGNGAANTGCAGCTCCACNGCGGCNGCGGCCCGCC
CGGGCAGGNACNCGNNTTCGTGGCGATANNGGANAGCCCGGTGAAAAGGGGCCNACAGGTCTTN
CTGGCTTAAAGGGACACA

| | |
|---|---|
| Sequence 358 | cMhvSD088e11 |

CTCCACCGCGGTGGCGGCCCGCCCGGGCAGGTACCGCGGGAAGGGCTGCTGTTTCGACGACACCG
NTCGTGGGGTCCCCTGGTGCTTCTATCCTAATACCATCNACGTCCCTCCANAAGAGGAGTGTGAAT
TTTACACACTTCTGCAGGGATCTGCCTGCATCCTGACGCGGTGCCNTCCCCAGCACGGTGATTAGT
NCCANAGCTCGGCTGCCACCTNCACCGGACACCTCATACACGCTTNTGCAGCTGTGCCTNGGCTCA
CAACACAGCATTGNCTGCTCTGACTTTGGACTACTCCAAAAATTGGCCTTAAAAANTTAAAAGGAG
ATCCGATACTTGNAAAGAAATACTAATAAACAAAACAGGNTTCCCTTTNGCGCGCTCTTATANACT
NGGNGGGAANCCCCCCGGGGCNTTGGCAGGGGAAATTTNCNAATTATTCAGANGCTTNNATTCTA
ATTNCCCGTCCNCACCTTCCNAAGGGGGGGGGG

| | |
|---|---|
| Sequence 359 | cMhvSD089h07 |

ATAGCTCCTAATTTAATTATTATAACAAAAATTTACTGAGCATCTACTATGGGCAAACATGGGAAA
TCTAAACATGCNTGAGTCCCAGTCCTAGCTCAGGATGACTTTANAACCTAANGGAAAACATAAAC
ATATACAGAAGGAACGTCAACCCAACATCAGAGTCTTTTTAANGGTTATATANAACATCCTTCAAG
ACNCCACANAANANCNCGCCTGANGGGGTGCCTGCCACAAAGGATGTGAGGGGTAAGCAGGGCG
GGCAGNATTTCCCAATCCCGCTGATCTCCACAACCATAGGAGGGGGCAGCTTCCNTTCCCCCATTC
CATATCAGTCTATTCATACNTTACAAGACAAAAGTNTGATTCCTTCCAANAAANAGTNTGCCANGG
ACCACNCACATACNGGATTTTACAGAATCTTTGAAATCATNTNTTTTCAACATTGTNATCGTTCAG
ATAAANAAAATGANATCAGGCCTNCACTGGCACTGAATCAAAGTNTTTGGGGAGATAGGCCCCAA
AAATTTNTTTAAAAAAATAAAAATG

| | |
|---|---|
| Sequence 360 | cMhvSD090c07 |

AGGTACGCGGGGGAGGAACTGCTCAGTTAGGACCCAGACGGAACCATGGAAGCCCCAGCGCAGC
TTCTCTTCCTCCTGCTACTCTGGCTCCCAGTTTCAGATGCCAGTGGAGAAATAGTGATGGCGCAGTC
TCCAGCCACCCTGTCTGTGTCTCCAGGAGAAAGAGCCACCCTCTCCTGCAGGGCCAGCCAGAGTGT
TAGCGGCAACTTAGCCTGGTATCAACATAAACCTGGCCAGGCTCCCAGGCTCCTCATCTATGGTGC
ATCCACCAGGGCCACTGGTA

| | |
|---|---|
| Sequence 361 | cMhvSD090c07 |

AGCAGTATAATCACTGGCCTTCTTTTGGCCAGGGGACCAAGCTGGAGATCAAACGAACTGTGGCT
GCACCATCTGTCTTCATCTTCCCGCCATCTGATGAGCAAGTTGAAATCTGGAACTGCCTCTGTTGTG
TGCCTGCTGAAATAACTTCTATTCCCAAGAGAGGGCCAAAGTTACCTGCCCGGGGCCGGCCGCTCT
TAGAACTAAGTGGGATCCCCCGGGCCTGCAGGAATTTCGATATTCAAAGCTTTATCGATACCCGNT
CGACCTCGNAGGGGGGGGCCCCGG

| | |
|---|---|
| Sequence 362 | cMhvSD092h01 |

GGCCGCCCGGGCAGGTACAATGCAAAAGATTCAAAGCCCCTTCCACTCTCTTCCAGTGTGCAAGAT
GAAAGAATGCATATGCTATTGCTTCACTGTCTCCTCTCTTCAGGATATGTTCTGGGGGTAGGATTA
AGCTTTTCATTTCTAGTAGGTATTTTGGCACATGAGGATTGAATTCCACAGCTCTATGAATGGGCCT
CTACTGGCATTCATCTCTTGCTGGTGCTCAAGCCCCCCGCCGAGAATGCCAGCCCTCAAGGAAGAA
GAAATTTTGTCAAGAAAAACAGCTCTTTGGCTTTTGGAGCCAAAAGCCAGCCTGGTGGTAAGCAAT
ATTTGGTTGGCTTGACCTTTTGGGTAAAGCCTTAATATCAATCAATACCTTTTGGCTTAAAGAACTT
GGNCCTGGAACCATTCAAGCCATTATTGCCTTTGNTAAGTTTCCCANNAAAGGGGCCTTTCTTAAA
AAANGGTTTTTCAATTGGGANTATTTGGAACCATACCTCAGAAANGGGGGGA

| | |
|---|---|
| Sequence 363 | cMhvSD093g05 |

AGGNACTTTTTTNTTTTTTTTTTTTTTTTTTNGGAATTATCTTGATTTCCTTTCACTACCAAGAAAAN
AAATACTTNAATNCNTTAGNNAATATTTTTGGGGTANNANAAAATTTTTAAGACNGTAGTTATGAG
TANNATGTGTATTCACAACAGNAATNTTCCCCCTGGNAGAGNGNGCTNANAATANACCTGCTNTG
GGNTAAAANANCTNNANGGCTTTGGACATTGCCTTTACATTCAAAAATGGAGTTCANTGTCATGGC
CNGAAAANANGNANTCCCCNAGGGAAAGCCAGGGAACCCNCCCGCTTNNAAAAGCNTTGGGCCT
TTAGGGAANAAAAGCNAGAAGAAGGCTTGGGGTTGCCNTTTCCCCCCACNCTGGATNTCCCCCAA
NCCTATTTTGGNTTTCTTGTTGAANGTTTCCAAAANCCNTNNCCNNAAAAACTTNTTGGGGGCCAA
AAGTTCACCTNTTANTACAANGCTTGNGGAANCCCCANTNTTNNTCCNCCCCGNTCCGTTTATGNA
GCCCAGNCAATTNAATNNGGGACCTTCCCTTGGGGCTTT

| | |
|---|---|
| Sequence 364 | cMhvSD093g12 |

TANCGTGGGNGCNGNCGAAGTNCTNNGTTAACTGCCTTTATATCATGCTNAAGTNNAANGCTAATT
TGAGTTTGAAATACNGTGGCTAATAGAGCTAANAAAACACATTCATCATCATTCTCTGGTATTNTC
TAATGTCTTCTGGTAGCTCCCACTCATCCCCAGAGTAGCCAAGGTTGAACTTGAACC

| | |
|---|---|
| Sequence 365 | cMhvSD094a09 |

AGGTACAAATTTGGAAAAAAATGCACACGGGTGGCAGGAAGACAAGCTATGATCTGCTCCAGGCA
TCAAGCTCATTTTATGGATTTCTGTCTTTTAAAACAATCAGATTGCAATAGACGTTCGAAAGGCTTC
ATTTTCTTCTCTTTTTTTTAACCTGCAAACATGCTGATAAAATTTCTTCACATCTCAGCTTACATTTG
GATTCAGAGTTGTTGTCTACGGAGGGNGAGAGCANAAACTCTTAAGAAATCCTTTCTTCTCCCTAA
GGGGATGAGGGGATGATCTTTTGTGGTGTCTTGATCAAACTTTATTTTCCTAGAGTTGTGGAATGA
CCAACAGCCCATGCCATTGATGCTGATCAGAGAAAAAACTATTCAATTTCTGCCATTTAGAGACAC
ANTNCNAATGNCTCCCATTCCCCAAAGGGTTCCAAAANGTTTTTCAAAATAAACCTGNNGGCAGCT
TCACCAAANGTTGGGGGGGAAAAGGCATTGAATTAGGTTTGGCANGGTTATGGTAAGGGANAAGG
GGTGAAGAATTTAAAAGAANNTTACNTACNTTTTNAANTTTTTAAAATTTANTTTTAAAGGTCNTA
AAAANTCCCATTNNGAAAAANNTTTTCCCCCNTTTTT

| | |
|---|---|
| Sequence 366 | cMhvSD015e12 |

GCCCGCCCGGGCAGGTACTTTCATNGNGTTNGNGATGTTNTNNTGNGACAGTGTCTCACTAGNGCA
GTGGCCGCTATCTTGGCTCACTGCAACCTCCTTCTNTTGGGTTCAAGTGATCCTCATGCTTCANAGA
TGGGG

| | |
|---|---|
| Sequence 367 | cMhvSD019e03 |

CNGGCCANGTACGCAGGGGGCCCCGNCGGNCATCGTTGAGCCCCGC

| | |
|---|---|
| Sequence 368 | cMhvSD026g08 |

ACGACTNCTATAGGGCGAATTGGAGCTCCCCGCGGTGGCGG

| | |
|---|---|
| Sequence 369 | cMhvSD026h12 |

CTNCTTAGGGCGAATTGGAGCTCCCCGCGGTGGCGGCCGCCCGGGCAGGTACAGAACTTAAGACA
CNACTATTNGNTGAGATGAANAAANGCATATATNGGANGCCTTCANAATGAAATGGTCAGAGGGN
GAGTTTACACAGATNGA

| | |
|---|---|
| Sequence 370 | cMhvSD029e08 |

GCTNTTATAAATGANTAAATANGCTAAGAATAG

| | |
|---|---|
| Sequence 371 | cMhvSD029f06 |

CCGGGCAGGTACTCTGCGTTGTTACCACTGCTTACTTTTTTTTTTTTTTTTTTTTTTTTT

| | |
|---|---|
| Sequence 372 | cMhvSD032c10 |

AGGGCGAATNGGAGCTCCCCGCGGTGGCGGCCGAGGTACCCGAATTTAATNCGAGTGGTCATCAC
AGTCCCCGAGGTGATGATGCTGGAGGCGT

| | |
|---|---|
| Sequence 373 | cMhvSD032f12 |

GGAGCTCCCCGCGGTGGCGGCCCGAGGTACTTTNTTTT

| | |
|---|---|
| Sequence 374 | cMhvSD040e06 |

CCGCCGTAATACCGACTCACTATTAGGGCCGAATTGGAGCTCCACCGCGGT

| | |
|---|---|
| Sequence 375 | cMhvSD040e10 |

CTCCCCGCGGTGGCGGCCGNCNGGCCAGGTACTTTTTTTTTTTTTTT

| | |
|---|---|
| Sequence 376 | cMhvSD041b10 |

AATTGGAGCTCCCCGCGGTGGCGGCCGCCCGGGCAGGTACTCCAGCCTGGGCGACAGACCAAGGC
TCTGTCTCAAAAAAA

| | |
|---|---|
| Sequence 377 | cMhvSD048e04 |

AATTGGAGCTCCCCGCGGTGGCGGCCGANGTGAGAGGATGGCTTGAGTCCAGGAGGTCAAAGCTA
CAGTGAACCATGTTTGTGTGGAGTGCCACTGCACTCCANCCCAGGTGACANAGCAAGACCGTGTC
ATAAAAAATAAACCACACNCAAANAGAGAANGATCTTTATGGATNAAAAAGATAATAATAATGT
GTATTTACTGAATGCCAATTATCTATCCAACCTGGTG

| | |
|---|---|
| Sequence 378 | cMhvSD053g06 |

AGGGCNAATTGGAGCTCCACCGCGG

| | |
|---|---|
| Sequence 379 | cMhvSD053g08 |

CCGCGGTGGCGGCCGAGGTACTTTTTTTTTTTTTTTTTTTTTTTTTGAGATAAGTCTCGCTCTGTCAC
CCAGGCTGGAGTGCAGTGGCATGATCTCGGCTCACTGCAAGCTCCGCCTCCTGGGTTCATGCCATT
CTCCTGCCTCACCTCGGAGTAGCTGGGACTACAGGCGTCCGCCACCGCGCCTGGCTCATTTTTTTTG
TATTTTTAGTAGAGACGGGGTTTCACGGTGTTGGCCAGGATGGTCTCGATCTCCTGACCTTGTGATC
CACCCGCCTCGACCTTCAAAGTGCTGGGATTACAGGCGTGAGCCACCGCGCCCAGCCGAGTTCAG
ACTATTTGGNGGGCAACAGCAAGACATGGTTTTTTAGG

| | |
|---|---|
| Sequence 380 | cMhvSD055f02 |

CCGCGGTGGCGGCCGCCCGGGCAGGTACTTTTTTTTTTTTTTTTTTTTTTGGAGATGGAGTCTTGCA
GTGTTGCCCAGGCTGGAGTGCAGTGGCACGATCTCAGCTCACTGCAAGCTCCACCTCCCGGGCTCA
AGCGATTCTCCTGCTCANCCTCCTGAGTAGCTGGGATTACAGGCGTGCGCCACCACGCCCAGCTCA
TTTTTGTATTTTTAGTAGAGACCGGGTTTCGCCATGTTGGTCAGGCTGGTCTCGAACTCCTGACCTC
GTGATCCGCCTGCCTCGGCCCCGCAAAGTGCTGGGATTACAGACGTGAGCCACCACGCCCAGCTG
GAAGTTAACTTT

| | |
|---|---|
| Sequence 381 | cMhvSD057e05 |

AATTGGAGCTCCCCGCGGTGGCGGCCGCCCGGGCAGGTACTTTTTTTTTTTTTT

| | |
|---|---|
| Sequence 382 | cMhvSD057g11 |

ATAGGGCGAATTGGAGCTCCCCGCGGTGGCGGCCGCCCGGG

| | |
|---|---|
| Sequence 383 | cMhvSD058f11 |

GGAGCTCCCCGCGGTGGCGGCCGNCCNNGCAGGTACTTTT

| | |
|---|---|
| Sequence 384 | cMhvSD063h09 |

GAGCTCCACCCGCGGTGGGCGGCCGCCCGGGCAGGTACGCGGGGCTTGAACCCGGAGTCAACAGA
GACTCCATCTCAAAAAAAAA

| | |
|---|---|
| Sequence 385 | cMhvSD067b08 |

CCGGGCNGGTNCTCAGACTACCACANATATTCCCTTACGGNCCAGGTCTCTCATGTTATGCTGTTTT
TTCCAACCTGAGCT

| | |
|---|---|
| Sequence 386 | cMhvSD070d03 |

CAGAATCCTGGCCAGGNNCCNAGGCTNNTC

| | |
|---|---|
| Sequence 387 | cMhvSD070h05 |

GGAGCTCCCCGCGGTGGCGGCCCGCCCGGGCAGGTACTTTTTTTTTTTTTTTTTTTTTTTTCCTTTTT
TTTTTTTTTTTTTTTTTTTTTTTTTTTTTTTTTTTTTTTTTTTTTCCTTTTTTTTTTTTTTTTTTTTTTTTTT

| | |
|---|---|
| Sequence 388 | cMhvSD074e01 |

TATAGGGCGAATTGGAGCTCCCCGCGGTGGCGGCCGAGGTACTTTNTTTTTTTTTTTTTTTTTTTCCT
T

| | |
|---|---|
| Sequence 389 | cMhvSD085b12 |

TGACTTTGATGTGTGACAACAGGCACCANCNATCGCCAACTAGANAAGCTCACCAGANCTCNGAT
GNNGGAAGCTTNTATNGGGGCCTCAGCAT

| | |
|---|---|
| Sequence 390 | cMhvSD086f10 |

CCGGGCAGGTACAGTGGTGTGATCTCAACTCACTGCAACCCTCTACCTCCTGGGTTCAAGTGATTC
TCCTGCCTCAGCCTCCTGAGCAGCTCANATTATAGGCACCCGCCAACATGCCCGGCTAATTTTNGT
ATTTTTAGTAGAGACGGGGTTTCACCATGTTGGCCAGGCTGGTCTCGAACTCTNGACCTCAGGTGA
TCCACCCGCCCCAGCCTNCCAAAGTGCTGGGATTACAGGCATGAGCCACCGCGCCTGGCCAAAAT
GAAGCATTTTTTTAAACCAAACTGTTTNTTTGCTAGNGTGATCTAGCCATGGNATTCATTCCACTGT
GCTCTATTTCTTT

| | |
|---|---|
| Sequence 391 | cMhvSD090c01 |

AAGCCTCAAGAGAGCAGACACGTGCTGAAAANNTNCTGNGCAGNCCNGATTNCCCTAAACTNTGG
TNAGTAACAGGTCTGCCTG

| | |
|---|---|
| Sequence 392 | cMhvSD014f05 |

CGCCCGGGCAGGGTACTTTTTTTTTTTTTTTTTTTTTTTTTTTTTTTTTTTTTTTTTTTTTTTTTTT

| | |
|---|---|
| Sequence 393 | cMhvSD074h03 |

CGCGGTGGCGGCCCGAGGTACTCGAGCCNNATGGAGTNGNNCNGCNCATCGANCAGACNCACGG
ACGTGTCCCAGGAGGAGACAAGC

| | |
|---|---|
| Sequence 394 | cMhvSD062h08 |

CGCCCGGGCAGGTACTTTTTTTTTTTTTTTTTTTTTTTTAAAAAANNATTTTTTTTTTNGCCCCNNGG
NNGNAAAAAAANANNNAATTNTAAANNNNNNNNNCCNNCCCCNNTNNGNNTAAAAANNATTTTN
TGCCNTANNCNNNNAAAGGGGGGGGNTTNTNNGNCCCCCCCNCCNCCCCCNNNTTNTTTTTTTTTT
TTT

| | |
|---|---|
| Sequence 395 | cMhvSC006f04a1 |

GCTGATTGGAGCTCCCCGCGGTGGCGGCCGAGGTACAAGCAGTAATTGATTCACTGGCCTTGGACT
ACTTGCAGGTCAGCTTGTCTCACATAACAGGTTGGTATATGTATAACTATCACATAATTATGCATTT
TAGTAAAAATAATTGTTTAGAACTGGCTTCGGGCAGTTGTGACCTCTAACTGTAATTTCCTTGCTTC
TTCTGTATGTTTCCACCTCTTGTGCTGTGCGCCTAGCCAAATCANAGTGCTCTTGATAAAAATTCTT
CTCAAATTTAGGCAGCTCATCAAGATTCCACTTCTTTTTAACTAATTTCTCCCCAGGGTTTCCAAAC
TTNTTTCCAGATAAGGGCCCTGCCCTACTTCCTCCAAATCGAGGTGCACCAAACCCTCGGTCC

| | |
|---|---|
| Sequence 396 | cMhvSC008h12a1 |

CGCCCGGGCAGGTACGCCGGGTGGCGTCACGCCCTCCCAGTGTGCAAATAAGGCTTGTTGTTTCNA
CAAACCCGTTCGTGGGTCCCTTGTGCTTNTATCTAATACAATCGACTTCCTTCCAGAAAAAGGAAG
TGTGAAATTTAAAACCTTNTTGANGGAATTTGCTTCANTCTTGACCCGGTGCCCGCCCCAACACGG
GTGAATAATTCCAAGANGCTCGGNTTGCAACTTCAACCGGAACACCTTAANAACACGCTTNTTCAG
CTTGTGCCTTNGGNTTAAAACAAAAAAATTGACTTGNTTCTGACTTTGACTACTTNAAAATTGGCC
TAAAAATTAAAAAGAAGAATCGATCCCAAAAAAAAAAAAAAA

| | |
|---|---|
| Sequence 397 | cMhvSC008c11a1 |

CCGGGCAGGTACTACTGCTGAGCTGACTGTCAAACCACAAGATGCAGTCCTTCCCACTCTTCCTCT
CCTTTCCAAAGGCAGAGGAGCCTCATCCCATAGCCGCCACCAGCCCTAGTATGAGGAGTACCTCG
GCG

| | |
|---|---|
| Sequence 398 | cMhvSC007d11a1 |

AGGTACCAGCTGTAACCAATACGATTCTGGGGCAGGTTGTGGGCGAGTAGAAGAACCTCCTTCCC
CTCTGCGACATTGAATGGCGTGGATTCAATAGTGAGCTTGGCAGTGGTGGGTGGGTTCCAGAAGGT
TAGAAGTGAGGCTGTGAGCAGGACCTCCTTCCAGGGGACATGCAATCTGCAGGGAGGGGCTGAGG
GGGGTCCCATGGTCTCTGCTGTCTTCTCTGTCCGCCTCTTTGTAGAGGAGCTTGAGCTCCAGGAATG
CTCTGGTCAGGGCTGCTGTGACTGTTGGCCCTGCTGTCCTTCCTCCTTCTGTCCCCGCGTACCTGCC
CGGGCGGCCGCTCGAGGGTCTTTGTCTTTCTTGGCCCGACTTTCCAGCGTCCTTCTTCTTCTTGTCGT
CCTTAGGCGGCATTGCGAAGCTCGGAGAATAGCTGCAGACACCGCAGCCTCGTCAAGATGTCGGA
CAAAAAAAAAAA

| | |
|---|---|
| Sequence 399 | cMhvSC008d09a1 |

TGGAGCTCCACCCGCGGTGGCGGCCGTTAAACATGTGTCACTGGGCAGGCGGTGCCTCTAATACTG
GTGATGCTAGAGGTGATGTTTTTGGTAAACANGCGGGGGTAAGATTTGCCGATTNCCTTTACTTTT
TTTAACCTTTNCTTTATGAACCATCCCTGTGTTGGGGTGAAAGTGAGGGTAAATAATGACTTGGTG
GGTGAATTGGAAAAATTGGGCTGGTAAATGNCAAGTCANTGGTTTAATCTGACCCCAGCTTATGCC
GGAGGAAAAAATGGTTTCAATGTTACTTATCCAACATTAATTCTTCTATTAGGGNGAANAGAATTG
GTCCCAATTGGGTGGTGAAGGAGGTCAATTATATGGTTNGGGAATTT

| | |
|---|---|
| Sequence 400 | cMhvSC008c05a1 |

AGGTACAACGCAGAGCAGGTCCTGAGTTGGGAGCCAGTGGCCCTGAGCAATAGCACGAGGCCTGT
TGTCTACCAAGTGCAGTTTAAATACACCGACAGTAAATGGTTCACGGCCGAGGTACTTGTTGTTGC
TTTGTTTGGAGGGTGTGGTGGGCTCCATTCCCGCCTTGACGGGGGCTTGCTATCTTGCCTTCCAGGC
CACTGTCACGGCTCCCGGGTAGAAGTCACTTATGAGACACACCAGTGTGGCCTTGTTGGCTTGAAC
TCCTCAGAGGAGGGCGGGAACAAGAGTGACCGAGGGGGCACCTTGGGCTGACCTAGGACGGTCA
AGCTTGGTCCCTTCCGCCGAACACCCAATTGGTGTCGGC

| | |
|---|---|
| Sequence 401 | cMhvSC008f05a1 |

AGGTACAGCAAAAACCCACCTGTGTAAACACACACAGCAAAGTGATGTAAGAAGTTTCCATATAA
AGGGCTGCAGTATGGGAGAGGTAATGTGCAGGCTGGTTGCGGTTGTAGGGGCCCACCTTACTGAA
CTTTTCCATGATATGGGACCTGCCCGGCCGGGCCGTCTA

| | |
|---|---|
| Sequence 402 | cMhvSC008h03a1 |

GAGCTCCCCGCGGTGGCGGCCGAGGTACACCAATTGAGGAGAGACACATGGGTGGGAAATTGCAA
TAAAAAGACGGCCCATAGCAGGCTGCATTCCCATGGCTGGCCAGAGGAGGAACGCTTTGTGTTCT
CATCGGAGCTGCATGGGAAGTCTGCATACAGCAAAGTGACCTGCATGCCTCACCTTATGGAAAGG
ATGGTGGCTCTGGCCTCCTGTGGCTGGCCTTGGTCTCCTGCATTCTGACCCAGGCATCTGCAGTGCA
GCGAGGTTATGGAAACCCCATTGAAGCCAGTTCGTATGGGCTGGACCTGGACTGCGGAGCTCCTG
GCACCCCANAGGCTCATGTCTGTTTTGACCCCTGTCAGAATTACACCCTCCTGGATGAACCCTTCC
GAAGCACANAGAACTCANCAGGGTCCCAGGGGTGCGATAAAAACATGAGCGGCTGGTACCTGCCC
G

| | |
|---|---|
| Sequence 403 | cMhvSC008f12a1 |

GGACCGAGGGTTTGGTGCACCTCGATTTGGAGGAAGTAGGGCAGGCCCTTATCTGGAAAGAAGTT
TGGAAACCCTGGGGAGAAATTAGTTAAAAAGAAGTGGAATCTTGATGAACCTGCCTAAATTTGAG
AAGAATTTTTTATCAANAGCACCCTGATTTGCTAGGCGCACAGCACAAGAGGTGGGAAACATACA
GAAGAAGCAGGGAATTCAGTTAGAAGGTCACAACTGCCCGAAACCCAGTTCTAACAATTATTTTA
CTAAAATGCATAATTATGTGATAGTTATACATATCCAACCTGTTATGTGAGACAAGCTGACCTGCA
AAGTAGTNCAAGGCCAGTGAATCAATTACTGCTTGTACCTGCCCCGGGCGGCCGCTCTAAACTAGT
GGATN

| | |
|---|---|
| Sequence 404 | cMhvSC006f03a1 |

AGGTACACACTGAAACCACTGTCAGATTAANAAACTACCACAACTTGTCTCAGNTNTTCAAACAAT
GAATCAAGTNCCNTGGNGNNGGCTGNNNATTAATCCTGTNTTGGCACTGCTGNTGGCTATNAAAC
TCACCNNCAAGGGTAAACGATNAAATTGAACCACCTGGTAGGNGTTATATTAACANATGATACTT
TTATTNTTGGAAANTCCAAGTTTGCTTNCTTGGTCTGNTGCAAGGGCAAANGNGGATNAGAAACC
ANGTNGCAAAGCNTGCTCTGGAGCATTGTCATTTNCCANTTTAATAACANGTACCTGCCCGGGCGG
NCGCCCGGGCAGGTACTTCACTGGAAATATGGGCGCCNAGGTGGCCTTCAACTGGATCATTGTTCA
CATGGAANANCCANATTTTGCTNAACCCACTNACCATGCCTGGTTATGGAAGGGCATCTTCTGCTN
GANCCTCTATTTNTGNTGCTTCTTGGACTGAATAACCAACCTCCAAAAAAAAATCTANCTATCATC
ACCTCCANTGGAATTTCANCNAAATCNAGCTATTTCAAAGCACTACCANCAACAAATAATAACCT
ACAAAAAAACACTTNCATNNGNATCTTTANCCACCCCTAAATT

| | |
|---|---|
| Sequence 405 | cMhvSC008d04a1 |

AGCTCCACCGCGGTGGCGGCCCGAGGTACGCGGGGGGCGCCATTTTGTCTCGGCAGCGGTGGCCC
GTAGCTCCATCGCATTTTATGTTTCTGGCGAGAAGGGAACGGAGTTTTCATCAGGTAGATTGGTTT
TGT

| | |
|---|---|
| Sequence 406 | cMhvSC009b06a1 |

GCTCNCCGCGGTGGCGGCNCGAGGTACAGCATTTCCTGGAGGATCTCTGGAGCGATATAGTCTGG
CGTGCCACAGAATGTGGCCGTGGTGACACCATTGCAAATCCCCTCCTTGCACATTCCGAAGTCTGC
CAGTTTACAGTGACCCTCGTGGTCCAACAGGACATTGTCCAGTTTCAGATCTCTCATACTCAGCCT
ATACCCCATCCTCCACTCTAGCACCCATCTCTACCCATCAGAGTCAGAATGAACACCCATAGGGGA
GGTGGCCACTGTGTGCCCCCCCGCGTACCTGCCCG

| | |
|---|---|
| Sequence 407 | cMhvSC009h03a1 |

AGGTACCAGGATGTCCAGTGCGACCATCTTTTCCAGCAGGGCCAGAAGGACCAGCAGGGCCCCTA
GGACCAGCAGGACCCACGGAGCCAGGAGCACCTT

| | |
|---|---|
| Sequence 408 | cMhvSC009h03a1 |

GGGCTCTCCCTTACCCGCGTACCTGCCCGGGCGGCCGAGGTACACGTCTCTGTCTGGGCCTCGGCC
AGGGTGCCGAGGGCCAGCATGGACACCAGGACCAGGGCGCAGATCACCTTGTTCTCCATGGTGGC
CATTGCCTCCTCTCTGCTCCAAAGGCGACCCCGAGTCAGGGATCCCCGCGTACCTGCCCG

| | |
|---|---|
| Sequence 409 | cMhvSC010e11a1 |

NATTGGAGCTCCCCGCGGTGGCGGCCGCCCGGGCAGGTACTGTCCAACTGGATGCTGCCCTGGTG
GCTGAAGGCACACTTCATGATGCTGTCCAGGGTCATCAGGGAGACATGTTGAAAGAGCTCCAGGC
GTGAGTTTTGGGCAATGTGTTCCTCCCATTTGTTCAGCATCATCCGAACACTCTCAGACATCATGGT
GATGAATATTTTCAGAATGCTGATGTTGAAGCCAGGTTTCACAATCTGGCGGTACCT

| | |
|---|---|
| Sequence 410 | cMhvSC016e09a1 |

AGGTACAAGCAGTAATTGATTCACTGGCCTTGGACTACTTGCAGGTCAGCTTGTCTCACATAACAG
GTTGGTATATGTATAACTATCACATAATTATGCATTTTAGTAAAAATAATTGTTTAGAACTGGCTTC
GGGCAGTTGTGACCTCTAACTGTAATTTCCTTGCTTCTTCTGTATGTTTCCACCCCTTGTGCTGTGCG
CCTAGCCAAATCAGGGTGCTCTTGATAAAAATTCTTCTCAAATTTAGGCAGCTCATCAAGATTCCA
CTTCTTTTTAACTAATTTCTCCCCAGGGTTTCCAAACTTCTTTCCAGATAAGGGCCCTGCCCTACTTC
CTCCAAATC

| | |
|---|---|
| Sequence 411 | cMhvSC016b09a1 |

AGGTACAAGCAGTAATTGATTCACTGGCCTTGGACTACTTGCAGGTCAGCTTGTCTCACATAACAG
GTTGGTATATGTATAACTATCACATAATTATGCATTTTAGTAAAAATAATTGTTTAGAACTGGCTTC
GGGCAGTTGTGACCTCTAACTGTAATTTCCTTGCTTCTTCTGTATGTTTCCACCTCTTGTGCTGTGCG
CCTAGCCAAATCAGGGTGCTCTTGATAAAAATTCTTCTCAAATTTAGGCAGCTCATCAAGATTCCA
CTTCTTTTTAACTAATTTCTCCCCAGGGTTTCCAAACTTCTTTCCAGATAAGGGCCCTGCCCTACTTC
CTC

| | |
|---|---|
| Sequence 412 | cMhvSC014g04a1 |

GGACCGAGGGTTTGGTGCACCTCGATTTGGAGGAAGTAGGGCAGGGCCCTTATCTGGAAAGAAGT
TTGGAAACCCTGGGGAGAAATTAGTTAAAAAGAAGTGGAATCTTGATGAGCTGCCTAAATTTGAG
AAGAATTTTTATCAAGAGCACCCTGATTTGGCTAGGCGCACAGCACAAGAGGTGGAAACATACAG
AAGAAGCAAGGAAATTACAGTTAGAGGTCACAACTGCCCGAAGCCAGTTCTAAACAATTATTTTT
ACTAAAATGCATAATTATGTGATAGTTATACATATACCAACCTGTTATGTGAGACAAGCTGACCTG
CAAGTAGTCCAAGGCCAGTGAATC

| | |
|---|---|
| Sequence 413 | cMhvSC027b01a1 |

AGGTACTGGCAAAAAAAGATGCTCGGTGGTTCCAGCAGAAGCCAGGCCAGGCCCCTGTGTTAGTG
ATGTATAAAGACAGCGAGCGGCCCTCAGGGATCTCTGAGCGATTCTCCGACTCCAGTTCACGGACC
ACAGTCACCTTGACCATCAGTGGGGCCCACGTTGAGGATGAGGCTGACTATTACTGTTACTGTGCG
GCCGCGGTCTCGGTCACTCGAATAACCCGACATGGCGTCAATGGTTGCGGTTGGCGGGGAACGAA
GTATATAGAAAAGCGTGCGACAAGTCGCTGGAAATGGCCTCGATGACGGCGAAGCCTTGCGGGGG
CGGCAGCGGAGGAA

| | |
|---|---|
| Sequence 414 | cMhvSC028f01a1 |

AGGTACAGCATTTCCTGGAGGATCTCTGGAGCGATATAGTCTGGCGTGCCACAGAATGTGGCCGTG
GTGACACCATTGCAAATCCCCTCCTTGCACATTCCGAAGTCTGCCAGTTTACAGTGACCCTCGTGG
TCCAACAGGACATTGTCCAGTTTCAGATCTCTCATACTCAGCCTATACCCCATCCTCCACTCTAGCA
CCCATCTCTACCCATCAGAGTCAGAATGAACACCCATAGGGGAGGTGGCCACTGTGTGC

| | |
|---|---|
| Sequence 415 | cMhvSC040c11a1 |

CCGCGGTGGCGGCCCGAGGTACTGGCAAAAAAATATGCTCGGTGGTTCCAGCAGAAGCCAGGCCA
GGCCCCTGTACTGGTGATTTATAAAGACAATGAGCGGCCCTCAGGGATCCCTGAGCGATTCTCCGG
CTCCAGCTCACGGACCACAGTCACCTTGACCATCAGCGGGGCCCACGTTGAAGATGAGGCTGACT
ATTACTGTTACTCTGAGGCTGACAACAATAGGGTGTTCGGCGGGGGGACCAAGCTGACCGTCCTA
GGTCAAGCCCAAGGCTGCCCCCTCGGTCACTCTGTTCCCGCCCTCCTCTGAGGAGCTTCAAGCCAA
CAAGGCCACACTGGTGTGTCTCATAAGTGACTTCTACCCGGGAGCCGTGACAGTGGCCTGGAAGG
CAGATAGCAACCCCGTCAAGGCGGGAGTGGAGACCACCACACCC

| | |
|---|---|
| Sequence 416 | cMhvSC033e12a1 |

ACTTAGGGCGAATTGGAGCTCCCCGCGGTGGCGGCCGAGGTACTACCCCTTCCCCAACCCCAGGG
AATGCAGCTCCTGACTCCAAAAGAGACCCTTCCTTCCTCTTGGGGAGAGGAGGGAGAAGAGTAAA
GAGGACTTTGTCTTGCATTGAAGTCCTCTTTGATGAGTGGGGATTCCTAGCTCCCAGAAACCATTTT
TAGAAACACCCTGGGCCAGAAGGGAACCTGCTGCCATGAAGGAAAGGACCCAGTCCTTGCGGAAT
ACGTCACCTGCTGACTAAAGATCCCTTGGGCCTTGAATAACCAGCAGCAATATCCAAGTAGTATAC
CATGGGCCTTGGGTGAAACTCTGAGACTTTCTGGCTCCAGGTGAAACCCAGCATATTGCCAGCTGT
GGTGGCTATAGTGAGAGACTTCTTCTGCTTGAGAAAAGCTGAAGGAAAAATAAAGCAGTATTTGC
CTTGTACCTGCCCG

| | |
|---|---|
| Sequence 417 | cMhvSC033a02a1 |

CTACTTAGGGCGAATTGGAGCTCNCCGCGGTGGCGGCCGCAGAAGGTCCCGGCAGCAGCAGGAAG
AAGACGGACCCCGCGATGAGGGCGGCGGCAAGGAGCACCTTCATGTTNGGTTCGGNAAGGCGCA
GCATCCCCGCGTACCT

| | |
|---|---|
| Sequence 418 | cMhvSC032f05a1 |

AGGTACACAAACCGTATGTTAAGTAGCGCAGCCAGCAGCTCACCACAGGGAAAAACAGCATCTGC
AAAAACGATGTCAAATCTTGACTCTTGTAGTTTTTTTCATAACTTTCTTATTTGAAACTACATCTTT
ACAGAAGTTTCTAAATATGTCATATAATTCCCACACGAGCGGCCGCCCGGGCAGGTACTTGTTGTT
GCTTTGTTTGGAGGGTGTGGTGGTCTCCACTCCCGCCTTGACGGGGCTACTATCTGCCTTCCAGGCC
ACTGTCACGGCTCCCGGGTAGAAGTCACTTATGAGACACACCANTGTGGCCTTGTTGGCTTGAAGC
TCCTCA

| | |
|---|---|
| Sequence 419 | cMhvSC031h07a1 |

CGAGATACTGTCCAACTGGATGCTGCCCTGGTGGCTGAAGGCACACTTCATGATGCTGTCCAGGGT
CATCAGGGAGACATGTTGAAAGAGCTCCAGACGTGAGTTTTGGGCAATGTGTTCCTCCCATTTGTT
CAGCATCATCCGAACACTCTCAGACATCATGGTGATGAATATTTTCAGAATGCTGATGTTGAAGCC
AGGTTTCACAATCTGGCGGTGCTTTTTCCATTTAGAACCATCCAGGGTCACAAGTCCTCGACCAAC
CCAGGATTCAAGGATTTTGTGGCTAACAGCACTTTTGGGATCTTGTCTTTTCAGGAGAATCTTGGC
ATAGTCTGGGTCATGGACACTGAAGAACATCGTAAAGGGTCCAACCCACAAGGGAACAGCACATG
GGTATTTTTCCATCAGCTTATGATACACCTCAAACTCCTTTACTGGGTAAAACTCCTTGTGGCCATA
GAACCAGTGGGCAGGGGGTGCAGGAAACAGGTGCAGGGCTCTGATCATCCATCTCCTCCTCTGGT
ACCTGCCCGGGCGGNCCGCTCTAGAACTAGTGGGATCCCCCGGG

| | |
|---|---|
| Sequence 420 | cMhvSC031g07a1 |

CCGCGGTGGCGGCCCGGGACCGAGGGTTTGGTGCACCTCGATTTGGAGGAAGTAGGGCAGGGCCC
TTATCTGGAAAGAAGTTTGGAAACCCTGGGGAGAAATTAGTTAAAAAGAAGTGGAATCTTGATGA
GCTGCCTAAATTTGAGAAGAATTTTTATCAAGAGCACCCTGATTTGGCTAGGCGCACAGCACAAGA
GGTGGAAACATACAGAAGAAGCAAGGAAATTACAGTTAGAGGTCACAACTGCCCGAAGCCAGTTC
TAAACAATTATTTTTACTAAAATGCATAATTATGTGATAGTTATACATATACCAACCTGTTATGTGA
GACAAGCTGACCTGCAAGTAGTCCAAGGCCAGTGAATCAATTACTGCTTGTACCT

| | |
|---|---|
| Sequence 421 | cMhvSC031c09a1 |

CGCGGTGGCGGCNCGGGACCGAGGGTTTGGTGCACCTCGATTTGGAGGAAGTAGGGCAGGGCCCT
TATCTGGAAAGAAGTTTGGAAACCCTGGGGAGAAATTAGTTAAAAAGAAGTGGAATCTTGATGAG
CTGCCTAAATTTGAGAAGAATTTTTATCAAGAGCACCCTGATTTGGCTAGGCGCACAGCACAAGAG
GTGGAAACATACANAAGAAGCAAGGAAATTCAGTTATGAGGTCACAACTGCCCGAAGCCAGTTCT
AAACAATTATTTTTACTAAAATGCATAATTATGTGATAGTTATACATATACCAACCTGTTATGTGA
GACAAGCTGACCTGCAAGTAGTCCAAGGCCAGTGAATCAATTACTGCTTGTACCTCGGC

| | |
|---|---|
| Sequence 422 | cMhvSC031b07a1 |

CCGCGGTGGCGGCCCGCCCGGGCAGGTACTGTCCAACTGGATGCTGCCCTGGTGGCTGAAGGCAC
ACTTCATGATGCTGTCCAGGGTCATCAGGGAGACATGTTGAAAGAGCTCCAGACGTGAGTTTTGGG
CAATGTGTTCCTCCCATTTGTTCAGCATCATCCGAACACTCTTAGACATCATGGTGATGAATATTTT
CAGAATGCTGATGTTGAAGCCAGGTTTCACAATCTGGCGGTGCTTTTTCCATTTAGAACCATCCAG
GGTCACAAGTCCTCGACCAACCCAGGATTCAAGGATTTTGTGGCTAACAGCACTTTTGGGATCTTG
TCTTTTCAGGAGAATCTTGACATAGTCTGGGTCATGGATATTGAAAGAACATCGTAAAGGGTCCAA
CCCACAAGGGAACGGCACATGGGTATTTTTCCATCAGCTCAGGATACACCTNAAACTCTTTTACTG
GGTAAGACTCCTTGGGGCCATAAACCAGTGCGCAGGGGGGTGCAGGGAAACCAGGTGCATGGCTT
CTGANCGGCCATCTCCTCCTCTGGTACCTTCGGGGCGCTTCTAGAACTAGTGGGATCCCCCGG

| | |
|---|---|
| Sequence 423 | cMhvSC031a08a1 |

GCAGGTACAGCCTGGGCTCCAGAGTCAGCCTCTACACTCACCAGACTATGGCGGATTCATCATTAT
ACTGGGAAGCNACAGCCTGGGCCCCANAGTTGGTCATCCGTNCATGCACAGATGAGGAGAGGTCT
CAGGANGCTTTGGCCGTGGTCTGGGACCTTACCTCTTTGTGTAATGAGTTGTTTGGTGTGAGGCCC
AGATNACAAGGGCCCCCNCNTACCTCGNN

| | |
|---|---|
| Sequence 424 | cMhvSC026c02a1 |

TAGGGCGAATTGGAGCTCCCCGCGGTGGCGGCCCGAGGTACTGCCTGGAGCACGACATCCAGCCC
AGTGGCACCATGCCCAGCCACAAGGCCCTGGGGAGCAGTGATAACTCCTTCAACACCTTCTTCAGG
GAGACCCAGCCTGGCAGGCATGTGTCCTGGGCTGTCTGTGGACCTGGAGCCTGCTGTCATAGGTTG
GCATCAACTACCAGTCCCCCACAGTGGTGCCCGGGGGTGCTGTAGCCAAGGTGCAGCGGGCAGTC
TGCGTGCTAAACAATACCACAGCCATCACTGAGGCCTGGGCCCGCCTCAACCAAAAGTTTGACCTG
ATGTATGCCAAGCGGGCATTTATGCACTGTTATGTGGACAGGGGCATGGAGGAAGGTGTCGAGCG
GCCGCCGGGCAGGTACTACAGCCTGGGTGACTGAGTGAGGCTCTTTCTCAAAAAAAAAAAAAAAA
AGAAAAAAG

| | |
|---|---|
| Sequence 425 | cMhvSC023f07a1 |

CCGCGGTGGCGGCCCGCCCGGGCAGGTACCAGAGGAGGAGATGGACGATCAGAGCCATGCACCT
GTTTCCTGCACCCCCTGCGCACTGGTTCTATGGCCACAAGGAGTCTTACCCAGTAAAAGAGTTTGA
GGTGTATCCTGAGCTGATGGAAAATACCTATGTGCCGTTCCCTTGTGGGTTGGACCCTTTACGATG
TTCTTCAATATCCATGACCCAGACTATGTCAAGATTCTCCTGAAAAGACAAGATCCCAAAAGTGCT
GTTAGCCACAAAATCCTTGAATCCTGGGTTGGTCGAGGACTTGTGACCCTGGATGGTTCTAAATGG
AAAAAGCACCGCCAGATTGTGAAACCTGGCTTCAACATCAGCATTCTGAAAATATTCATCACCATG
ATGTCTAAGAGTGTTCGGATGATGCTGAACAAATGGGAGGAACACATTGCCCAAAACTCACGTCT
GGAGCTCTTTCAACATGTCTCCCTGATGACCCTGGACAGCATCATGAAAGTGTGCCTTNAGCCACC
AGGGCAGCATNCAGTTGGACAGTACCTT

| | |
|---|---|
| Sequence 426 | cMhvSC023c06a1 |

GACTACTATAGGGCGAAATTGGAGCTCCCCGCGGTGGCGGCCCGAGGTACAGGACATTCCTCTGC
TCCTATTGCCCCTGTTTCCGTTCTTTTCACACTGTCTGTGGGTGCTGTGCCCTGTTGGAACTCTCTTT
AACGTCTTACGTTGGAGCCGCTAACCTTCCCCAGGTGTTTGTCTTCATTGCTTTCACAGGGAAAGA
ATTACTCGTCCCACTGACGAGTTCTATGTATGTCCCTGGGAAGCTGCATGATGTGGAACACGTGCT
CATCGATGTGGGAACTGGGTACCTGCCCGGGCGGCCGAGGTACGCGGGAATGAGGCCATTGCTGA
ACTTGATCACTGAATGAAGACTCATACAAAGACAGCACCCTCATCATGCAGTTGCTTAGAGACAA
CCTAACACTTTGGACATCAGACAGTGCANGAGAAAGAATGTGATGCGGCAGAAGGGGCTGAAAA
CTAAAATCCATACAGGGTGTCATCCTTCTTTCCTTTAAAGAAACCTTTTTACACAATCTTCCATTC

| | |
|---|---|
| Sequence 427 | cMhvSC025f05a1 |

GACACGGCTTCCTGGGCGGTCCCCTCCACCTGTTGCTTCAGGTCCTGCAAGCCCTTGCTTGCCATGG
CTTCGGGGTATCTGTGGAGTCGTCAAGAGCAGCTGGAGCGACGTTGGATCCTGCCCAGAGTGGCC
CCCGCGTACCTCGGCCGCCCGGGCAGGTACAAGCTTACAAAACTCAGACCACTCACCAGAAAAAA
ATCGGCATTTATATAGTTGTGTTACTTTTGGTTTCCTGCATCTTTTCACATCTGGCTCATTTACATCA
TTTTCTTCATCTTCCAAAGTGGAGTTAGCTACTACATTAGGTAAGGTTACTTCATCAATCACCATAC
TGTTATAATCTTGAAAGTGAATTTCTTTGGACCCTCCCTTGAATGCAGTTATACCTAGTAAACCTGA
TCCACAACCAAGATCCAAGACTTTTTTCCCAGCAAATTTCACTTTGGCCTTTGTGAAATAAAGCCA
GGAGGGNAAAAGGGTCCT

| | |
|---|---|
| Sequence 428 | cMhvSC025a04a1 |

CGGGCAGGTACAAGCAGTAATTGATTCACTGGCCTTGGACTACTTGCAGGTCAGCTTGTCTCACAT
AACAGGTTGGTATATGTATAACTATCACATAATTATGCATTTTAGTAAAAATAATTGTTTAGAACT
GGCTTCGGGCAGTTGTGACCTCTAACTGTAATTTCCTTGCTTCTTCTGTATGTTTCCACCTCTTGTGC
TGTGCGCCTAGCCAAATCAGGGTGCTCTTGATAAAAATTCTTCTCAAATTTAGGCAGCTCATCAAG
ATTCCACTTCTTTTTAACTAATTTCTCCCCAGGGTTTCCAAACTTCTTTCCAGATAAGGGCCCTGCC
CTACTTCCTCCAAATCGAGGTGCACCAAACCCTCGGTCC

| | |
|---|---|
| Sequence 429 | cMhvSC034e05a1 |

ATAGGGCGAATNGGAGCTCCCCGCGGTGGCGGCCGAGGTACGCGGGAAGATCTACACTATTATGT
CACCCCAGAAAGTGAACTCTCAGTCTTCCCAGCCAGTCTCTTTCTTATCATAGGTTAGCTTGCTTAT
TCTGGAATTTCGCGTATACAGATGCATGCCATGCCATAGGTACCTGCCCG

| | |
|---|---|
| Sequence 430 | cMhvSC030g10a1 |

CCGCGGTGGCGGCCGAGGTACACCGACTACGGCGGACTAATCTTCAACTCCTACATACTTCCCCCA
TTATTCCTAGAACCAGGCGACCTGCGACTCCTTGACGTTGACAATCGAGTAGTACCTGCCCGGGCG
GCCGCCCGGGCAGGTACTCTTGCTGCTTGGTTGATTAATAAAGCGGGACGTCCCTTTGAGCAGCCT
CAAGAATATGATGACCCTAATGCAACAATATCTAACATACTATCCGAGCTTCGGTCATTTGGAAGA
ACTGCAGATTTTCCTCCTTCAAAATTAAAGTCAGGTTATGGAGAACATGTATGCTATGTTCTTGATT
GCTTCGCTGAAGAAGCATTGAAATATATTGGTTTCACCTGGAAAAGGCCAATATACCCAGTAGAA
GAATTAGAAGAAGAAAGCGTTGCAGAAGATGATGCAGAATTAACATTAAATAAAGTGGATGAAG
AATTTGTGGAAGAAGAGACAGATNATGAAGAAAACTTTATTGATCTCAACGTTTTTA

| | |
|---|---|
| Sequence 431 | cMhvSC022e05a1 |

TCGAGCGGCCGCCCGGGCAGGTACCCTTGCTGATGTGGGTCTTCAGCTCCTCTTCTGAATACTCCA
CCTTGGGCCTTTTGTTCCAGAACCTTCATTATCGTGTTTTCTCTTGGTAACTTTCCCTTCAGGATTGT
AATCTGGTGGGTAAACAAGCTCCTTAAACTCATCCACCAAGGAGCCCAGTCTTTTATTCATTGCTT
CAACCTTGGGCAATGTCAGGTCCACTGCTTGTTCCGGCTCCATCAAATCCAAGGCCAAGGCCTCCA
GGTTCCTGAAGTGCTGCTGCAGCACGGGGTTCTCAAAGCTGTCACTTCTGTACCTCGGCCGAGGTA
CAAACTCGCATTCATGGCTTGGTTTCCCAGAAGATCTCCATTTAACTTTTTTAAAGAAAGTTTATTG
CTTTCTTTAACCTGCATTTTTTCTAAGTTTTTTTTCACATAAAGGTGCTGTCTTTGTGGCAAGGCCTA
NGCATGACAATCGGAGGACTCGAGGGGGAT

| | |
|---|---|
| Sequence 432 | cMhvSC022d03a1 |

CCGCGGTGGCGGCCGTTAAGGACAGTTGTGGCAAAGGAGAAATGGTCACAGGGAATGGGCGGCG
GCTCCACCTGGGGATTCCTGAGGCCGTGTTTGTGGAAGATGTAGATTCCTTCATGAAACAGCCTGG
GAATGAGACTGCAGATCAGTATTAAAGAAGCTGGATGAACAGTACCTCGGCCGGCTGTTATGTTC
ATCATGGCACTTAAGAGATGCTTAACAAACCTTTCCTACAATGTTCCTCAGATTTTCAGAGCTTATT
TGATCTAGCATCTGGTTCCTAAATTCTGAGTCACATCAGAAGCCAAACTTGAATGCTTTTGGAAAG
AGCTAGCCTCATACCACTTCAGTTGGGAAGGGGAGTACCT

| | |
|---|---|
| Sequence 433 | cMhvSC027c04a1 |

GGAGCTCCCCCGCGGTGGCGGCCGAGGTACTGTCCAACTGGATGCTGCCCTGGTGGCTGAAGGCA
CACTTCATGATGCTGTCCAGGGTCATCAGGGAGACATGTTGAAAGAGCTCCAGACGTGAGTTTTGG
GCAATGTGTTCCTCCCATTTGTTCAACATCATCCGAACACTCTCAGACATCATGGTGATGAATATTT
TCAGAATGCTGATGTTGAAGCCAGGTTTCACAATCTGGCGGTGCTTTTTCCATTTAGAACCATCCA
GGGTCACAAGTCCTCGACCAACCCGGGATTCAAGGATTTTGTGGCTAACAGCCTTTTGGGATCTTG
TCTTTTCANGAGAATCTTGGCATTANTTTGGGTCATGGGACACTGAANAACATCGTTNAGGNTTCA
NCCCACAGCGGGAAACAGCACATGGGTATTTTTNCATCAGCTTATGATACACCTTCAAACTNCTTT
ACTGGGTAAAACC

| | |
|---|---|
| Sequence 434 | cMhvSC027e11a1 |

CCGCGGTGGCGGCCGAGGTACCAAAAAGACTCTCAAAAACCAATACTCCCACGGGCAAGGGAATA
GCCAAGTTTGTTGCGGTTTCCAATGAATGACATCAGCCCTGTGTAGGTCTCAATCAAAATGGGTTC
AGTTAACACCATCAGTTTTTTCCTCTTCCAGATCCAGTTGAATTCTTGTGGGCATTCTGGATAGCTG
GAACAAGCTTAGACATGAACCCAGACAACTTGCAAATTTCAAGGAATTTCTCACTGGTGTATTTCA
TAGGATGCTCAGTGAAAGTAGCATAAGGAACTTCAGTGGACCATGGGTTCCAGCGGGACAGAAGA
GGCTGCTCCTCCGGACTCCCCCAGTAGATCCTAAGGCCTTCTCCTTGTCTCTTGTCCAGGGACATCC
CAGGGAAGGTGAACTTGCCCAGGCAGATGCGATAGACAGCGCTCAGAGGAATCCGCTTGCAGCTG
CACACAACTCAGCATGATGAAGTCGTATTTGCAGATCAAGGAGAAGTCTTGTTGTGACCAGTAAG
AATTCTCTCCTTCTCATTGNTCCAGTGGGTCTATCTTTGTCAAGAGCCAGAAGCCTTGAATGGTCTT
TTCAGAAGTCTTAACTTCCGTGACCTTTCAAGTCTTTCATGGCAGTCTTAATGGGCCCCCNGGCCGN
TCTAGAACTAGTGGGATCCCCCGGGCTGCAAGGAATTTNATTACAAAGCTTATCGATNCCGGCNA
ACCTCNAGGGGGGGC

| | |
|---|---|
| Sequence 435 | cMhvSC027e09a1 |

CGCGGTGGCGGCCCGCCCGGGCAGGTACAGGGCAGTAATTGATTCACTGGCCTTGGACTACTTGC
AGGTCAGCTTGTCTCACATAACAGGTTGGTATATGTATAACTATCACATAATTATGCATTTTAGTA
AAAATAATTGTTTAGAACTGGCTTCGGGCAGTTGTGACCTCTAACTGTAATTTCCTTGCTTCTTCTG
TATGTTTCCACCTCTTGTGCTGTGCGCCTAGCCAAATCAGGGTGCTCTTGATAAAAATTCTTCTCAA
ATTTAGGCAGCTCATCAAGATTCCACTTCTTTTTAACTAATTTCTCCCCAGGGTTTCCAAACTTCTTT
CCAGATAAGGGCCCTGCCCTACTTCCTCCAAATCGAGGTGCACCAAACCCTCGGTCC

| | |
|---|---|
| Sequence 436 | cMhvSC037e10a1 |

CCGCGGTGGCGGCCGAGGTACTGTCCAACTGGATGCTGCCCTGGTGGCTGAAGGCACACTTCATG
ATGCTGTCCAGGGTCATCAGGGAGACATGTTGAAAGAGCTCCAGACGTGAGTTTTGGGCAATGTG
TTCCTCCCATTTGTTCAGCATCATCCGAACACTCTTAGACATCATGGTGATGAATATTTTCAGAATG
CTGATGTTGAAGCCAGGTTTCACAATCTGGCGGTGCTTTTTCCATTTAGAACCATCCANGGTCACA
AGTCCTCGACCAACCCANGATTCAAGGATTTTGTGGCTAACAGCACTTTTGGGATCTTGTCTTTTCA
GGAGAATCTTGACATAGTCTGGGTCATGGATATTGAAGAACATCGTAAAGGGTCCAACCCACAAG
GGAACGGCACATAGGTATTTTTCCAT

| | |
|---|---|
| Sequence 437 | cMhvSC037f08a1 |

CGGCCCGAGGTTATCGTTAGGCATCTCCCANGCGACCGGCTCCGCAGCAAGATGGCGGACGAGAA
GGACAGGGAAGAGATAATAGTAGCAGAATTTCACAAAAAAATCAAAGAGGCATTTGAAGTCTTTG
ACCATGAGTCGAATAATACAGTGGATGTGAGGGAGATTGGAACAATTATCAGGTCATTAGGATGC
TGTCCTACGGAAGGAGAGCTGCATGATCTGATTGCAGAGGTAGAGGAAGAAAGAACCTACTGGAT
ACATTCCGATTCGAAAAATTTCTTCCCGTGATGACAGAAATACTACTAGAAAGAAAATACAGACC
AATTCCAGAAAGATGTCCTTCTTCNAGCTTTTGAGGTTTTAGATTCAACTAAACCTGGGTTTCTTAC
TAAGGGCCGAGCTGATCAAGTATATGACTGAAGAAGATGGAGTTTCNCTCCCTCGCCCAGCTGAA
ATGCCAGTGGCGTGATCTTGGCTCGTTGCAACCCTCACCCTCCCGGTTCAAGCCATTCTTCCTGCCT
NAANCCTTCTGAGCAACTGGGATTGGNAGGCCACACCCAACACNCCTGGCTAAATTTCTGTATTTT
TNGGGANAA

| | |
|---|---|
| Sequence 438 | cMhvSC037e07a1 |

CGTGGCCCGTGGCTCACGTGGCCCCTAAGTTTCCGGGTCTTCCTCAGTCTGGATGGCATGTTGGCA
GCCCAGACGAAAAAGCCCCGCGTACCTNGGCCGNNNAAANNTTNTNNATCCTCCGGGCTG

| | |
|---|---|
| Sequence 439 | cMhvSC038b12a1 |

CCGCGGTGGCGGCCGTTAAACATGTGTCACTGGGCAGGCGGTGCCTCTAATACAGGTGATGCTAG
AGGTGATGTTTTTGGTAAACAGGCGGGGTAAGATTTGCCGAGTTCCTTTTACTTTTTTTAACCCTTC
CTTCCCGCGTACCT

| | |
|---|---|
| Sequence 440 | cMhvSC038b12a1 |

AGGAATTTCGATATCCAAGCTTATCGAATACCCGTCGACCTCGAG

| | |
|---|---|
| Sequence 441 | cMhvSC038g09a1 |

ANCACTACTTAGGGCGAATTGGAGCTCCCCGCGGTGGCGGCCGAGGTACGCGGGGACCTCATTCA
TTTCTACCGGTCTCTAGTAGTGCAGCTTCGGCTGGTGTCATCGGTGTCCTTCCTCCGCTGCCGCCCC
CGCAAGGCTTCGCCGTCATCGAGGCCATTTCCAGCGACTTGTCGCACGCTTTTCTATATACTTCGTT
CCCCGGCAAACCGCAACCCATTTGACGCCAATGTCGGGGTTATTCCGAGTTGACCGAAGACCGCG
GC

| | |
|---|---|
| Sequence 442 | cMhvSC038a03a1 |

CCGCGGTGGCGGCCGCCATGGAGCAGCCGCCGGCGCCTAAGAGTAAACTAAAAAAGCTGAGTGA
AGACAGTTTGACTAAGCAGCCTGAAGAAGTTTTTGATGTATTAGAGAAGCTTGGAGAAGGGTCTT
ATGGAAGTGTATTTAAAGCAATACACAAGGAATCCGGTCAAGTTGTCCNCAATTTAANCAAAGTC
CCTTGGGCCGCTCTTAGAAACTAGTGGGATCCCCCGGGCTGCAG

| | |
|---|---|
| Sequence 443 | cMhvSC038d02a1 |

CCGCGGTGGCGGCCGAGGTACACGTCTCTGTCTGGGCCTCGGCCAGGGTGCCGAGGGCCAGCATG
GACACCAGGACCAGGGCGCAGATCACCTTGTTCTCCATGGTGGCCATTGCCTCCTCTCTGCTCCAA
AGGCGACCCCGAGTCAGGGATCCCCGCGTACCTGCCCGGGCGGCCNGTTNNAAAAACTANTGGAT
CCCCCGGGCNTGCAGGA

| | |
|---|---|
| Sequence 444 | cMhvSC039b01a1 |

CCGGGCAGGTACGCGGGGAGGCCGTAGGAGGAAGATGGCGGTGGAGTCGCGCGTTACCCAGGAG
GAAATTAAGAAGGAGCCAGAGAAACCGATCGACCGCGAGAAGACATGCCCACTGTTGCTACGGGT
CTTCACCACCAATAACGGCCGCTCTNGAACTNGTTGGATCCCCCGGGCCTGCANGGAATTC

| | |
|---|---|
| Sequence 445 | cMhvSC038h11a1 |

CTTAGGGCGAATTGGAGCTCCCCGCGGTGGCGGCCGTGCATCATCATGGAGTTAGTGAGGCGCTCC
ACAATGGGACACTGAGCTTTGCGGAAGCGTTTGGCGGCATACCGCCCTGCACTGTGAGGCAGGTA
CCTGCCCG

| | |
|---|---|
| Sequence 446 | cMhvSC038h11a1 |

TATTTNAATNNCCCGTCCACCCTTCGAGGGGGGGGGGNCCGGGTACCCAGC

| | |
|---|---|
| Sequence 447 | cMhvSC038h11a1 |

ATTGCNCGCTTGGGCGTAAATCATGGGTCAT

| | |
|---|---|
| Sequence 448 | cMhvSC038h11a1 |

CGCTCCACAAATTTCCACACCAACATACCGAANCCGGGGGAGCCANTAAAAAGTTNTTAAAAGCC
CTGGG

| | |
|---|---|
| Sequence 449 | cMhvSC038d08a1 |

ACTATAGGGCGAATTGGAGCTCNCCGCGGTGGCGGCCGCCCGGGCAGGTACCCAATAGTGGATGG
GAAGCTTTCCATCCAGTGCTACTTGCGGGCCTTGGATCGATGTTACACATCATACCGTAAAAAAAT
CCAGAATCAGTGGAAGCAAGCTGGCAGCGATCGACCCTTCACCCTTGACGATTTACAGTACCTCGG
CCGCTNTTAAAACTAGTTGNATNCCCCCGGGCCTGCANGGAATTCCGATATCAAAGCTTTATCGAT
ACCGTC

| | |
|---|---|
| Sequence 450 | cMhvSC038g06a1 |

CGACTNCTTAGGGCGAATTGGAGCTCCCCGCCGTGGCGGCCGCCCGGGCAGGTGCTGTGAGTGCT
CTGGCGAAGTTTGGAGCCCAGAATGAAGAGATGTTACCCAGTATCTTGGNGTTGCTGAAGAGGTG
TGTGATGGATGATNNNNATGAANTAAGGGACCGAGCCACCTTCCACCTAAATGTCCTGGAGCAGA
AGCAGAAAGCCCCNTTAATTCNAGGCTTNTATCCTAAAATGGTCTGACTGTTGTCCATCCCTGGTC
TGGAGAGGACTCTGCAGCAGTACCTNGGCCGCCCGGGCAGGTACAAAATGATTTCCCAAAGTTCT
TGAAGTGCCTTGAGAACATGTGGGTCCGAGTTGTTATAACAGACTCNTCCCCCGGGTCACCTTTTG
CCTGGTCATNCTGTTAGAGTACCTTTGGCCGNTCTANAACTAGTGGGATCCCCCGG

| | |
|---|---|
| Sequence 451 | cMhvSC038g05a1 |

CGACTACTTAGGGCGAATTGGAGCTCCCCGCGGTGGCGGCCGAGGTACGCGGGGAGGAGGTCGAG
AGTCGTTCTTCTCTTTGCACAGACGTGACTCTGCAGCTCTTTAACGGNGCCCGCTGCTCTCAACCCA
GCTTACCCCACTTTNTCCNATGGC

| | |
|---|---|
| Sequence 452 | cMhvSC038g05a1 |

TTNAAACTTTNTTCNATACCCGTCCGACCTCGA

| | |
|---|---|
| Sequence 453 | cMhvSC038g05a1 |

ATTTGTTTATCCCGCTCACAATTCCACACAAACAATACCGAAGCCCGGGGAAGCCATAAAAAGTGT
AAAGGCCTTGGGGGTGCCTAATGGAGTGAGCTTAACTCACATTAATTGCGTTGCCGCTCACTGCC

| | |
|---|---|
| Sequence 454 | cMhvSC038f05a1 |

AGGTACCTTCTGGGGCATACAACGTGGCAGCAGGGCCTCGGGAAGAGGGGTAGGAGGACCGAGC
AGCAANNGNGTGTCTTAGGAAGACAGGAAAAAAAAACCCTTTTGNCACACATGCNNGGAGGGTT
GTCCCTGAAAAGAAGGGCAGGTTGGGANAGGTNCCCCTNGTNNCNTTTAANAAAAAAAGGCCCCC
CAGGTGGGCCAAAANAAGCCACCNANTTNAAANGTAGGGGAATTGAATCNATATAAAAAAAAAC
AAAATCNACCGCCCANAAANTANANGGGAACCAAAATTCAATCCTTTTCCACCGGGTTTTCNTTTT
CCCAACCCAAGAAAAA

| | |
|---|---|
| Sequence 455 | cMhvSC021g12a1 |

AATTGGAGCTCCCCGCGGTGGCGGCCGGGACCGAGGGTTTGGTGCACCTCGATTTGGAGGAAGTA
GGGCAGGGCCCTTATCTGGAAAGAAGTTTGGAAACCCTGGGGAGAAATTAGTTAAAAAGAAGTGG
AATCTTGATGAGCTGCCTAAATTTGAGAAGAATTTTTATCAAGAGCACCCTGATTTGGCTAGGCGC
ACAGCACAAGAGGTGGAAACATACAGAAGAAGCAAGGAAATTACAGTTAGAGGTCACAACTGCC
CGAAGCCAGTTCTAAACAATTATTTTTACTAAAATGCATAATTATGTGATAGTTATACATATACCA
ACCTGTTATGTGAGACAAGCTGACCTGCAAGTAGTCCAANGCCAGTGAATCAATTACTGCTTGTCC
TCGGCCGCTCTAGAACTAAGTGGATC

| | |
|---|---|
| Sequence 456 | cMhvSC021f11a1 |

CGAATTGGAGCTCCACCCGCGGTGGCGGCCCGCCCGCCATGGGACCACGTGGGGTAAGTTGGGTT
GAGAGCAGCGGGCGCCGTTAAAGAGCTGCAGAGTCACGTCTGTGCAAAGAGAAGAACGACTCTCG
ACCTCCTCCCCGCGTACCTCGGCCGCTCTAGAACTAGTG

| | |
|---|---|
| Sequence 457 | cMhvSC021f08a1 |

CGCCCGGGCAGGTACAGCCTGGGCTCCAGAGTCAGCCTCTACACTCACCAGACTATGGCGGATTC
ATCATTATACTGGGAAGCAACAGCCTGGGCCCCAGAGTTGGTCATCCGTCCATGCACAGATGAGG
AGAGGTCTCAGGAAGCTTTGGCGTGGTCTGGGACCTTACCTCTTTGTGTAATGAGTTGTTTGGTGT
GAGGCCCGGTCACAAGGGCCCCCGCGTACCT

| | |
|---|---|
| Sequence 458 | cMhvSC021a08a1 |

CGCGGTGGCGGCCCGCCCGGGCAGGTACACTGCCAAACCCGCAGAAGTGCCCAGGGAAAGCCCCG
CGGGGGCTGCGGATAGTCACGGCTGATGGAAAGCTGACAGCGGAACAAGGACGCAACGTCACTCT
CATGGTGCAATTAGAAGAGGGTGATGTTCAGCCGGACACTCATCCAAGTGGACTTTGGCGATGGT
ATCGCGGTGTCTTACGTCAATCTCAGCTCCATGGAAGATGGGATCAAACACGTNTATCAGAACGTG
GGCATTTTCCGTGTGACCGTGCAGGTGGACAACAGTCTGGGTTCTGACAGCGCCGTNCTGTACCTT
CGGC

| | |
|---|---|
| Sequence 459 | cMhvSC021a08a1 |

TGATATCAAGCTTATCGATACCGGTCNACCTCTAGGGGGGGCCCNGGNCCCAACTTTTTGTTCCCT
TTAG

| | |
|---|---|
| Sequence 460 | cMhvSC021f07a1 |

TTAGGCGAATGGACTCCACGCGGTGGCGGCCGTCCGGGCAGGTACCAGGATGTCCAGTGCGACCA
TCTTTTCCAGCAGGGCCAGANGGACCAGCAGGGCCCCTAGGACCAGCAGGACCCACGGAGCCAGG
AGCACCTT

| | |
|---|---|
| Sequence 461 | cMhvSC021f07a1 |

GAATGCCTTGTGGGCCACTAGGACCTCTTGGGCCAACCCCGCGTACCT

| | |
|---|---|
| Sequence 462 | cMhvSC017a08a2 |

CGAATTGGAGCTCCCCGCGGTGGCGGCCGCCCGGGCAGGTACCTGCGGAGGCAGCGGCTGCTGCG
GGACCTGCGCCCCTTCCCAGCGCCCCCCACCCACTGGTTCCTTGGGCACCAGAAGTTTATTCAGGA
TGATAACATGGAGAAGCTTGAGGAAATTATTGAAAAATACCCTCGTGCCTTCCCTTTCTGGATTGG
GCCCTTTCAGGCATTTTTCTGTATCTATGACCCAGACTATGCAAAGACACTTCTGAGCAGAACAGA
TCCCAAGTCCCAGTACCT

| | |
|---|---|
| Sequence 463 | cMhvSC018f05a2 |

GGCGAATTGGAGCTCCCCGCGGTGGCGGCCGCCGGGCAGGTACTTTACTGCACCCAGCAGACTTTC
AACAACTCATTGATCCAAAGATACATGCACAGTCTGAGCACCAGCTATGGTGCTCATAACTTCTTT
AAGACTTGAACCCTTTCAATCTGTGTGATTCATTAAATTGGACCATTGATGATAAGAATACACATT
GTATGTTTCTGTGCACATGACAGTGTGTGTGTGTGCACGTACCT

| | |
|---|---|
| Sequence 464 | cMhvSC018d07a2 |

AGGTACTGGCAAAAAAAATATGCTCGGTGGTTCCAGCANAAGCCAGGCCAGGCCCCTGTTCTGGT
GATTTATAAAGACGGTGAGCGGCCCTCAGGGATCCCTGANCGATTCTCCGGCTCCAGTTCACGGAC
CACAGTCACCTTGACCATCAGCGGGGCCCACCTTGAGGATGAGGCTGACTATTACTGTTACTCTAC
GACTGACAACAATGGGGTGTTCGGCGGAGGGACCAAGCTGACCGTCCTACGTCAGCCCAAGGCTG
CCCCCTCGGTCACTCTGTTCCCGCCCTCCTCTGAGGAGCTTCAAGCCAACAAGGCCACACTGGTGT
GTNTCATAAGTGACTTCTACCCGGGAACCGTGACAGTGGCCTGGAAGGCAGATAGCANCCCCGTC
AAGGCGGGAGTGGAGACCACCACACCCTCCAAACAAAGCAACAACANGTACCTGCCCG

| | |
|---|---|
| Sequence 465 | cMhvSC018c08a2 |

ACATGGATGGCTCTCAAGACAGCCCTATCTTTATGTATGCCCCTGAGTTCAAGTTCATGCCACCAC
CGACTTATACTGAGGTGAGGATTGTCATCTTTACTGTTAAATTTGTCCTAAGCTTTCTATAAGAAGT
TGACTTAGACGGATTGCTAAACTGGTTTGTTCTTTTTGTTCTTACCTGAACTGAAATAGTCTGTTTC
TTTCTTTAGGTGGATCCCTGCATCCTCAACAACAATGTGCAGTGAGCATGTGGAANAAAAGAANC
AGCTTTACCTACTTGTTTCTTTTTGTCTCTCTTCCTGGACACTCACTTTTTCAGAGACTCAACAGTCT
CTGCAATGGAGTGTGGGTCCACCTTAGCCTCTGACTTCCTAATGTAGGAGGTGGTCANCANGCAAT
CTCNTGGGCCTTAAA

| | |
|---|---|
| Sequence 466 | cMhvSC036d11a2 |

GCGCGTAATACGACTACTATAGGGCGANTTGAANNTNNANNCGGCCGAGGNACCTTGATCTCCTG
GCGGNGGCTCGTCCCTGGTCTTAGTTCCACCGGGCNGCGGGAGTCAGGACCGCCTGTCCTCAGACC
CCTCCGCAGCGACT

| | |
|---|---|
| Sequence 467 | cMhvSC019a09a3 |

CCGCGGTGGCGGCCGAGGTACTTTTTTTTTTTTTTTTTTTTTTTTTATTTTTTTTTTTTTTTTTTTTTTT
TTTTTTGCTCTAAAGGGGGTAGAGGGGGNGCTNTAGGGTAAATACNGGCCCTATTTCAAANATTTT
TAGGGGAATTAATTTTAGGACNATGGGCATNAAACTGNGGTTTGCTCCACAAATTTCAAANCATTN
TCGAGCGGCCNCCCGGGCAGGTACTTNNTTTTTTTTTTTTTTTTNGGNGGNAATNTTGNTTTGTNNC
CCAAGCTGGAGTGCANTGGCATGGTNTTTGGTTAANTGCAACCTTCACCTTTCCTAGTTTAAAGCN
ATTTTNTNCTGCCTNANNCCTCCCCTAANNAGCTTGGNGATTACAGGNAANATGCCCCCCAATAGC
CNGGGNAAAATTTTTTGGAATTTTTAGCAAAAAAANAAGGGTTTTTCNCCATTGCTTGGCCCANGG
CTTANNNTTTAAAAANTTNCCTGNCCCTTTAAAGNGGAATCTTGGNCCNCCNTTTGGGNCCGTTTT
TTAAAAAANTNGNTNGGAATTCCCCCCCGGGGCTTTGGAGGGAAAATTTTNAATTTTNCAAANCCT
TTATTTTAATTCCCNGNCNNANCCTTTGAGGGGGGGGGGGC

| | |
|---|---|
| Sequence 468 | cMhvSC020b11a3 |

AGGTACTGTCCAACTGGATGCTGCCCTGGTGGCTGAAGGCACACTTCATGATGCTGTCCAGGGTCA
TCAGGGAGACATGTTGAAAGAGCTCCAGACGTGAGTTTTGGGCAATGTGTTCCTCCCATTTGTTCA
GCACCATCCGAACACTCTCAGACATCATGGTGATGAATATTTTCAGAATGCTGATGTTGAAGCCAG
GTTTCACAATCTGGCGGTGCTTTTTCCATTTAGAACCATCCAGGGTCACAAGTCCTCGACCAACCC
AGGATTCAAGGATTTTGTGGCTAACAGCACTTTTGGGATCTTGTCTTTTCAGGAGAATCTTGGCAT
AGTCTGGGTCATGGACACTGAAGAACATCGTAAAGGGTCCAACCCACAAGGGAACAGCACATGGG
TATTTTTCCATCGGCTTATGATACACCTCAAACTCCTTTACTGGGTAAAACTCCTTGTGGCCATAGA
ACCAGTGGGCAGGGGGTGCAGGAAACAGGTGCAGGGCTCTGATCATCCATCTCCTCCTCTGGTAC
CTGCCCGGGCCGGCCGCTCGAAGGTACGCGGGTGAAGAAAAGGCTCTAACATGAGTTTGATCTTG
AGCCCCAATGTTGAACAAGCTTCCAGACCTTTACAATTTTAA

| | |
|---|---|
| Sequence 469 | cMhvSC029b09a2 |

TTGAGGAGAGACACATGGGTGGGAAATTGCAATAAAAAGACGGCCCATAGCAANGCTGCATTCCC
ATGGCTGGCCAGAGGAGGAACGCTTTGTGTTCTCATCGGAGCTGCATGGGAAGTCTGCATACAGC
AAAGTGACCTGCATGCCTCACCTTATGGAAAGGATGGTGGGCTCTGGCCTCCTGTGGNTGGCCTTG
GTCTNCTGCATTCTGACCCAGGCATCTGCAGTGCAAGCGAGGTTATGGAAACCCCATTGAAGCCAG
TTCGTATGGGCTGGACCTGGACTGCGGAGCTCCTGGCACCCCANAGGCTCATGTCTGTTTTTGACC
CCTGTCAGAATTACACCCTCCTGGATGAACCCTTCCGAAGCACANTANAACTCAGCAGGGTCCCAT
GGGTGCGATAAAAACATGAGCGGCTGGTACCTGCNCG

| | |
|---|---|
| Sequence 470 | cMhvSC029b09a2 |

NGGAATTTAATATCAAGCTTATNGATACCCGTTCTAACCNTNGGANGGGGGGGGCCCCGGTACC

| | |
|---|---|
| Sequence 471 | cMhvSC020b10a3 |

TGTGGGTGAGTTGGCTGCCGGTGAGTTGGGTGCCGGTGGAGTCGTGTTGGTCCTCAGAATCCCCGC
GTAGCCGCTGCCTCCTCCTACCCTCGCCATGTTTCTTACCCGGCCTGAGTACCTCGGCCGCCCGGGC
AGGTACTGTTTTGAGGAGAAGGATCAGCTATCCAGCGACTGTGAGCATGAACAAGAGCCAAGCCT
AGAGACATAATCATCTTGACCCTCTGAGTTACAGGATTCGGCTTATTTTCTTCTTCTTCTAAAACTC
GGGCAAAATGGCTGAGCTGCCAAATTGGACGACCCTCGCGGCTTTCCCGAGAAAGCTCTAATACC
AAGGACACACAAGCTGGGAAGAAAGTCATGAACACGAAGTANTTGGCAAGAACTGACATGCAGC
CAAAGCAGCACATAATTTCAAGCTGACCGTACCT

| | |
|---|---|
| Sequence 472 | cMhvSC012h12a2 |

CCGGNCAGGTACGCGGGGGCTGTANGCTCAGGAGGCAGAGCTCTGAATGTCTCACCATGGCCTGG
ATCCCTCTCCTGCTCCCCCTCCTCATTCTCTGCACAGTCTCTGTGGCCTCCTATGAGCTGACACAGC
CATCCTCAGTGTCAGTGTCTCCGGGAGAGACAGCCAGGATCACCTGCTCAGGAAATGTACCTCGGC
CGAGGTACGCGGGGGCACTTGGCTTCAAAGCTGGCTCTTGGAAATTGAGCGGAGAGCGACGCGGT
TGTTGTAGCTGCCGCT

| | |
|---|---|
| Sequence 473 | cMhvSC001g01a2 |

AGGTACCTGCAGGCCTCCTACACCTACCTCTCTCTGGGCTTCTATTTCGACCGCGATGATGTGGCTC
TGGAAGGCGTGAGCCACTTCTTCCGCGAACTGGCCGAGGAGAAGCGCGAGGGCTACGAGCGTCTC
CTGAAGATGCAAAACCAGCGTGGCGGCCGCCCGGGCAGGTACTTGTTGTTGCTTTGTTTGGAGGGT
GTGGTGGTCTCCACTCCCGCCTTGACGGGGCTGCTATCTGCCTTCCAGGCCACTGTCACGGCTCCC
GGGTAGAAGTCACTTATGAGACACACCAGTGTGGCCTTGTTGGCTTGAAGCTCCTCAGAGGAGGG
CGGGAACAGAGTGACCGAGGGGGCAGCCTTGGGCTGACCCAGGACGGTCAGCTTGGTCCCTCCGC
CGAATACCACATAAATACCTT

| | |
|---|---|
| Sequence 474 | cMhvSC035h10a2 |

CGGGACCGAGGGTTTGGTGCACCTCGATTTGGAGGAAGTAGGGCAGGGCCCTTATCTGGAAAGAA
GTTTGGAAACCCTGNGGAGAAATTAGTTAAAAAGAAGTGGAATCTTGATGAGCTGCCTAAATTTG
AGAAGAATTTTTATCAAGAGCACCCTGATTTGGCTAGGCGCACAGCACAAGAGGTGGAAACATAC
AGAAGAAGCAAGGAAATTACAGTTAGAGGTCACAACTGCCCGAAGCCAGTTCTAAACAATTATTT
TTACTAAAATGCATAATTATGTGATAGTTATACATATACCAACCTGTTATGTGAGACAAGCTGACC
TGCAAGTAGTCCAAGGCCAGTGAATCAATTACTGCTTGTACCT

| | |
|---|---|
| Sequence 475 | cMhvSC035c03a2 |

CCGCGGTGGCGGCCGCCCGGGCAGGTACTTTACTGCACCCAGCAGACTTTCAACAACTCATTGATC
CAAAGATACATGCACAGTCTGAGCACCAGCTATGGTGCTCATAACTTCTTTAAGACTTGAACCCTT
TCAATCTGTGTGATTCATTAAATTGGACCATTGATGATAAGAATACACATTGTATGTTTCTGTGCAC
ATGACAGTGTGTGTGTGTGCACGTACCT

| | |
|---|---|
| Sequence 476 | cMhvSC001e01 |

CCGGGCAGGTACTANAAGCTGGGGGAAAAAGAGTNGGTNAAACANACATGGCCTTGGCCCTTCTG
GAATTTACATTCTCGTATGTGTCATGAAAGTTGTTTTGAAAAAACCCAAACCATNGTTTTTNCTNTG
CTTTCACACTACAACAATCAACACAGAAGACTTCTGTGACTCCAAAAAATATGTAAGGATTTCTCC
CCACCACCAGGCAAGCAATCAGTTCTGCAGCGGACACCAGTTGGGTGTTCTNCAATTCAATTNCAA
CACTATCTACCTAGAGACAGCATCAGATCCCACAGCATGAGGGCTCAATGCCCAAGCTGCCCCAC
AGCCCCCTGGGCACCAGTAGCAAGTCTGGGCCTCTGGAACTTCTTTTTTTGCAGAGATGGGGTCTC
ACTATATTGCCCAGACTGGGGGCTCA

| | |
|---|---|
| Sequence 477 | cMhvSC006b01 |

CACCGCCGGTGGCCGCCGGCTTGTTATTGCTCATCATGGCACTTAAANAGATGCTTAACAAACCTT
TCCTACAATGTTCCTCAAATTTTCAGAGCNTNNNNGNNGGGAGCATCTGGTNCCNAAAAAAAAAA
TTCTTTTNAAGCCAANCTNGAATGCTTTTGGAAAGAGCTAGCCTCATACCACTTCANTTGGGAAGG
GGGAGTACCTCGNCCCCTCTAAAAACTAATGGGATCCCCCCNGGCCTGCCAANA

| | |
|---|---|
| Sequence 478 | cMhvSC010d11 |

TGATGTATAAAGACAGCGAGCGGCCCTCAGGGATCTNTGAGCGATTCTCCGACTCCAGTTCACGG
ACCACAGTCACCTTGACCATCAGTGGGGCCCACNTTGAGGATGAGGCTGACTATTACTGTTACTGT

| | |
|---|---|
| Sequence 479 | cMhvSC010d11 |

AGCTGTTTCCTGANNCNCTNAAACTNNCNAANGAANGCATTTTTTAAANANCTTNGNTTTTNGGCC
TNNTTAAAACCAATTTAAACNTNTNTGAANTTTTNGGATTTTAA

| | |
|---|---|
| Sequence 480 | cMhvSC010h12 |

TGGAGCTCCCCGCGGTGGCGGCCGAGGTACTTTTTTTTTTTTTTTTTTTTTTAANNCNNTTTTTTTTT
TNTNTTTTTTTTTTTNTTTTTTTTTTTTTTTTTTTNTTTTTTTTTTGTNCCCNANCCNANNANGACNNT
NNNNTTNTTTNTTNANAAAAANNAAAAANAANGCCCNNNNTTATNNNAAAAAAAAAAAANATNT
TTTNTTTNCNCTCCNNCANNNCCNGANGANGNNNGGGNGNTNNCGNGANAAAANAATNNGAGGG
GGNTTNNNCAANAANAAANGTNCCNCCCCTNTTANCANTTTGAANNANGAAAGGGGCNGGATNTT
GGAAGCTGTGAGANNNTCCCCGAGGAACCTCCTGCNNTTTCTTCTCCTGAAGNGCTTATGAAGGG
GCGANCATNNTNCTCCATACATNCNNATTTCNTATAGNGNNCCCAAAGGGACCCACCTTNCTCCCT
NGAAATTTGGCTTAAAGCAACAAATAAAGTTTTTTTTTTGGNGGGGAAGGGAAANGGCTCTTTTTN
CTTGCTGTTTCNAAAATGNGGNGAACCCATTTNATGTTTCTGGGGGGAGGAANNCCCCCCNGGGG
NNAATTNNAANAAAANAAANCCCCCCCNCCGGNNAAAAAAANTANTTNNATNANATANNNNNCC
CNAAAAGGGGGGGGGGGCCCCCCCCCCCTTTTTT

| | |
|---|---|
| Sequence 481 | cMhvSC011h06 |

TGGAGCTCCACCGTGGTGGCGGCCGAGGTACAACGCAGAAGCAGGGTCCTGAGTTGGGAGCCAGT
GGGCCCTGANCANTANCACGAGGCCTGTTGTNTACCAAGTGCAGTTNAAATACACCGACAGTAAA
TGGTTCACGGCCGCCCGGCAGGTCAGTGCCCCCCGTGAAAGACAGAATTGTGGTTTTCCTGGTGTC
ACGCCCTCCCAGTGTGCAAATNAGGGCTGCTGTTTCGACGACACCGTTCGTGGGGTCCCCTGGTGC
TTCTATCCTAATACCATCGACGTCCCTCCAGAAGAGGAGTGTGAATTTTAGACACTTCTGCAGGGA
TCTGCCTGCATCCTGACGCGGTGCCGTCCCCAGCACGGTGATTAGTCCCAGAGCTCGGCTTGCCAC
CTCCACCGGCACCTCAGACACGCTTCTGCAGCTGTGCCTCGGTTACAACACAGATTGACTGCTCTG
ACTTTGACTACTCAAAATTGGCCTAAAAATTAAAAGAGATCGATNCCAAAAAAAAAAA

| | |
|---|---|
| Sequence 482 | cMhvSC012a10 |

ACGNNCCTNTNTNTNCAGGCCATGGNAAAAAAAATCCAATTATAGACCGTCTTGAGAGTGTGGTC
TTGCTTCTTATGTAGTATNAANTTNGAGAACTGATAATTAATGCATNGATTNACNTTNTTNAACNN
ATTNAATNNTAATTGTGAAAAAANAATTCNANGCACNNATNGTNAAATTGAANANNANANNAGG
ANATTTAAGACCTTGAGGAGCTNGAGCCGGNCATTATNTTAAATGTGAGGGGTTTATGACACNGT
ACCCTNCAATGGTGTTNACTANNCTTNNGNANATGNACATGCNNNCNATNNTNCNCATTGNNNCT
TAAGGCGTTTGGGGTCACACAGTNTTNAANGTNNTAGAAGACCNGTCCCCTAGGAGTNCCCNTGA
TTTCATCTNAACATCTTTGCTGATGCTCANAGGTACTTTTGCCAAGCANTAAAAGATCCAGGTATA
TAGCANNTAGTTGNGGTGTCATGTACTGCAAACATGCAAACAGTTTTTTNAANTTCANCCTTGGGC
AGAATCTNCTTTCAATAGAAAAGTNCTTTTGGCGTTTTTCNACTTTTTGNGNAACTCCAANANAGT
TNTTGTTCCCAGA

| | |
|---|---|
| Sequence 483 | cMhvSC012b02 |

CCGGGCAGGTACNCCATTGAGNGCTNTNNTNCCTTAGCNACNAGGNNGNNNCTGGNNANNNGAA
ANNTCACTAAANTGNANTTANNANTNNAGNNNAACNNGNNNNTNNTGTNNNTCATNCATGAANN
TTNCANCTNTTANNCTNTTNTGNNNGGNCTGCCCNTTNTTCTANACGTGGATGGTGGAATAACCAT
TGATCTGAGCNAACCTTTATTGTGANCAACTANTGAANAAGGNCAANCNTGTCTTANTANNNNGA
NGGAANAGCTNCATCTCNACANCNAAACAAACCATCAAGGTTTGCCACTTGTTGAAATTTGNNGC
CACAACTNCNGACTACACTGACTTGACAATTAAACCCACTCCCCTTTTNAAGGGTTTCCTTCCGNT
AAAAGATTGGGAAGANGGCCATATTATNCAACAAACTCATTANATCCCCGTNACAGTACGAGTAN
NCTATATGNAAACTACCANTTGGGCTTTGATTTTNATTCGTAACGCATTGCTTTTTTTTNTGNANCA
NTNNTACACTNCATTTTTTAAGAATTCAANTNTTTAAAAATTNGTTTGCTTTTCCTTAANGAAATTC
ATCCNGGCCAAGGAATAAGGGGGGGNGTTTTANTNGGAATTNTAAGGGCCAAAGGNTTNCNCCCC
NCAATAAAAATTGNTTGCTACAAACTTNACNNCAANAAAAAGAGTTTTGGGNNCNTTTNTNCCCC
AAAAAAATNNAAACNTCCAACCNANNNATTCNTAANCTCGNNTTTNNAANGTNCTAACAANTTTT
AGGGANTTTTTTTTTTT

| | |
|---|---|
| Sequence 484 | cMhvSC012b04 |

AGGNNCCCNTATTNGNNTTTTTGNNANACANTCCATGGANAAACNGGTGGAGCTGCNCCNAGGCN
CTGANCNTGNCNCCCTCTACTGNANTAACTNTANNCACGACTNNTACTTACTCTGNGCTNGNNGTG
ANAAGGGNACNTGNCCGGGCGGCCGACGTACNGGTGCTCTCCAGGCTGGCAGCCCGCTGCCTA

| | |
|---|---|
| Sequence 485 | cMhvSC012f09 |

ACGNGCCNGGNACAGTGGNANGANNANGGCCCNCNNTNNNATTTNCCTNNCNGGCCTAAGNNAN
TNTNTNACTTGCAGCCTCCCAATTATCTGGGACTACNGGCGCATGCAACCATACNTGGNTAATTCN
TGTATNTNTTGTGGAGACAGCATGTGGCTGTCTCTACATANCTCATGNTGTCCGCCCAGGCACAGT
GATTAAACTCCCNGGCTCANGTGATCCTNCTGCCTGGGCNTGNNAANNTGCTNGGATTACAGGCA
TATGCCAGCNTGNNCTGNCTTTCCTGTATTTNGTAATNTAGGAANTGGGAGTTCATGNTGGGAGGC
ACATTNCCTATAGGACTCCNGNNCAACCTACGNTGAAAATANGTATTCCTANAAAANGGNTTNTA
CNNACTNATATTACGGGGCACCANTATTGNTATCAACCTGAGAATGCTTTTTACATTATTTNGAGN
AGAACCTACGTGTNATTCANATAGTAAAAACTCAAACCCTAAANCNGAGTGAGAGCANCNTANGN
TTCANGTTTTCTAATATCCTTAAGATTTTCCTTTGCTTCC

| | |
|---|---|
| Sequence 486 | cMhvSC014d02 |

CCGGGCAGGTACTTGNGGAANTCATGCCTGGAAGGGGCTTGGGCACNTNANTAAGNCNGCCNTNN
TTTNGNTAAAAGGAGGGAAAAATCTACTTGAATTGACTTACCANANGCTTGATAACAGAGATGNC
TAGGATTAAAATCCNGATANTGACAAATCCACCCNNAAATCCCATCTTCTANTNTNATGNCCCCCC
GCCTNCCTGANTCGCTNTNNAACNNNATGGATNCCCCGGGNTCTAGGAANGGGNNNTNAAGCNNA
TCTATNCCNNCCNNCTCTGANGGGGGGCCCNGCACCCAGCTTTTAGTNNCCTTNNATAGGGGNTTA
ATGNGCGCGCTTGGCGTAATCATGGT

| | |
|---|---|
| Sequence 487 | cMhvSC016e01 |

GCTCCACCCCCGGTGGCGGCCACAGGAGCACATNTCCCTCTTCTNNAGGTGTGTCCCTCAGCATGA
CGCTGACTGATGTGNCATAAAGACTGACTNGTGACACTGGCTAGTGCTNNCNAGCCATCTAGACT
ACAACTTATTCTAGATACACCCTGGAGAGATCTTAAAGNGCATATCTNNTTCACCCANAGAAGGC
ATTTATGCCTT

| | |
|---|---|
| Sequence 488 | cMhvSC017a07 |

ATTGGAGCTCCCCGCGGTGGCGGCCGCCCGGGCAGGTACTTTTTTTTTTTTTTTTTTTTTNGNAAAA
NNGGGGGGNAAAAANTCCCCNANTNNNTTTANTTTTTTNAANCCNNNCTTNAAAANNCCCCCNNN
NNGGGGNNNNCNGGGGGGGAAAAAAANACNTGGGGGGAAANNAAAAANTTGGGCCTTAAANNN
CAANCNNANGNTTTNAANNNNNCCCCNGNTTTTNCNGNAAAAAAANTTTTNNTNNNTCNNNAAAN
AAAAAATTGNTCTNTNNGGGGGAAAAAAANGNCCCCNNNGGGGGGGNNNNNNCCNAATTNTTTN
NGGGGNNTTTTTAANGNGGGGGGGNNGAACCCAAANCCNNTTTNNAANGGGGGGGNTTTTTANN
NCCNNANNGGGGGNAAAAATTTTTNCCCNCNNNNNNNGGGTTTTTNCCAANGNNNAAAAAAGNN
TNNCNTTTTNGNNNAANNCNTAAATTTCCNGGGGNTTTTANNGGGTTTNGGGGCCAANTNAAANG
GAAANNAAAAATTTTTTTTGGANNANNCNTTTTNCCCCCGGCNGGGNGGGTTTCCCCCCCCCCNAA
AAATTTCCCACATTTTTTNNCNNAAAAAGGGGGGGCCTTTTAACCNTNCAAANNANCCCCCNTGGGT
TNNGGGGGTNAANANTNGGGCCCCCCNAAAAGTTNTTTNAANAAAANNNTTTTNNAAANGGGNN
GGGGGNCCCCCCCTGTTTATTAAATNGGGAAACCNNNAAAACNNGGNGGTTNAAAAAAAAGGGA
NCCCCCGGGNGGGAAATTNNTANNAANTTTTTNNANCCCCCCCC

| | |
|---|---|
| Sequence 489 | cMhvSC019c02 |

NNNGNGCNGGTANCTTGGNCGGTNTTNACGGGNTTCNTGNTCATGGNGNNNNGGATNACGTGATA
CTAGACAAAAANNCCATTCCNNCCNAGNATGTCTTGNGCNNGGCGGGCGATNNNCANGGCTTTNC
NACANGTATTNCTCTNCAGCAGANAAACCATNTTNGNGGCAGNCTTGNNCNGNNCCTTNAAGCAN
CCGCTNTAAAACTANNGGATNCNCNGGNCTGNANGAATA

| | |
|---|---|
| Sequence 490 | cMhvSC019c03 |

AGGTACANNGNNACNTANTTCNTTNTTNCCNAACNNNAANNTNGCNGNTGNTGNTGGTGTNATAT
GTGNACTTACTCCGCTGNCGACCNCTCANGGNTATATCCAAATCGAGGCCATTTATCAGCGACTGA
GTCAGGACGCTTATCTATATANTTTAACCCCCTNCNNCCNAAACCATTGACGCCATGNATGGGTTA
TNCGCAGTGACCGACAACCGAATTCGCTCTAT

| | |
|---|---|
| Sequence 491 | cMhvSC019d01 |

ACNTACTNGGTNCNNCTNTNTTANGAGGGTGNNNATGGACACCACTCCAGGTCTTGATGCTCTAG
GTATCTCACCTTCCATCCACACATGTTCACGTGGGTCNCGACTANAATTCACTCTATAGAGACACA
CACAGATGTAGGCCTTGNTGNTCTTGAATGCTTCTCAATTACTGANTGGCGGGATAACATGAGCNT
ACTCCGAGGANGGGCNTGGCNTTNTGNGCTCNACCCTAGGTACTGACAAGATTGGATNNCCTCCN
CCNAACACCCAATTGGTTGTAAATGCGCTNTAGAACTAGTGGATCCCCTNGGGCTGCATTTAATTC
GATATCAAGCTTATCTATTACCAACTAACCTAT

| | |
|---|---|
| Sequence 492 | cMhvSC021a02 |

ACGCCCGGGCAGGTACTTTTTTTTTTTTTTTTTTTTTTTTCCCTATCGATCTCTTTAAATTTTTAGGCC
AATTTTGAGTAGTCAAAGTCAGAGCAGTCAATCTGTGTTGTGAGCCGAGGCACANNTGCAAAAGC
GTGTNTGAGGTGTCCGGTGGAGGTGGCANCCNANCTNTGGGACTAATCACCGTGCTGGGGACGGC
ACCGTGTNAGGATGCANGCAGATCCCTGCAAAAGTGTNTAAAATTCACACTNCTNTTCTGGAGGG
ACGTCGATGGTATTAGGATAGAANCACCAGGGGACCCCACNAACGGGGTNGTNGAAACAGCACN
CCTTATTTTGCCCACTTGGGAGGGGCNNTGACACCAAGAAAACCANATTTTTTGTTTTTTCACGGG
GGGGCCANTNTACACGTTTNTGTNTTGGGCCTTGGGCCGCTNTANAACTAAGGGGATCCCCCGGGC
T

| | |
|---|---|
| Sequence 493 | cMhvSC029d07 |

GTTCTGAGCCTCAGCTGACCATANTGCTCATGCCAAGTCCTGAGCAGGGCATNTTGAATGGTGGTT
CCCTCATGACTACATACACCGTTAGGGAATGTTTCGTTAAGAGGAAATCAAGATGTTCTAACCTGT
GAAGGTAGAATAGATTCCAGGCTACACAAACACATGAAGTGTGCCTTATATTGATTACTAAAGAG
GTTGCTGCCAAGACTGCTTCCAAAGGGCAGAANATAGCCCTAAAAAATGTTTGCAGTGTGGAAAT
GCATTTTTAATAAGTCATATTCTAGTAACAAGTTGCATTTGGTAAGACACAAAGAAACAATGTTGG
TNTGCAGAGTAGAAATCTCTGGAAGATGATATTGTCATATCAGAGATATTGTCAGTATCAGGAGAT
ACCTTGAAATCTCTGGAAAGATGATTTTTTTGTCTCACATATGGCATTNCACAAANTAANAATGCC
CAAAAACTTGCAAAAATTCACCCCCGTACCTCCNGGCCCGCTTNTTAGAAACCTANTTNGGGATCC
CCCCGGGGCCTGCCAGGGAAATTTCNATTATTCAAAGNCTTTATNGGATACCCCGNTNCTACCCTT
CCNANGGGGGGGGGC

| | |
|---|---|
| Sequence 494 | cMhvSC031e09 |

CGCCCGGGCAGGTGCGAGAATGAAGACTATTCTCAGCAATCAGACTGTCGACATTCCAGAAAATG
TCGACATTACTCTGAAGGGACGCACAGTTATCGTGAAGGGCCCCAGAGGAACCCTGCGGAGGGAC
TTCAATCACATCAATGTAAGAACTCAGCCTTCTTGGAAAGAAAAAAAAAAGAGGCTCCGGGTTGA
CAAATGGTGGGGTAACAGAAAGGAACTGGCTACNNGTTCGGACTATTTGTAGTCATGTACCTCGG
CCCGAGGTNCTTTTGCTNTCTGCCTTTGCCAATATTTACTTTGGATCTTTTGTTTTTTGCCNTTTATT
TNGTTTTTTGCCTCTGNTTTAAAACANGCCTAATTTNNGAAAGGGCAATAAGNGAANGCTTGCNAG
TAATACATTGCTGAAAAATGCNANTTCACCAGAAAAATCAAGCAATTNGATTTTCTTTANGAATGA
AGTGCCTAGAAGTTGGTNCTGTNGGCNATTCAGAGGGTNAAAAATNGANNTAACNAATGGGGCCA
GGGACTTCCTGCCTTGGATGGACNTANATTCCAAACACCNNNTTTTGAAACACTNGGATTTTCAAN
ACCACNACCANATGGATGATAAAATGGANTNGNTTTACCACNCCTTANTANCACCACCAACAACC
TANATTGTGGGTTAGNCCAAATGGAAAAAGAGAAACNTGGTNANTACTTCCTTTGGGNTGCTAAA
TTGGGAAAANAAAA

| | |
|---|---|
| Sequence 495 | cMhvSC035b03 |

AGGTACAACAGGCTTCAGATGTTACTATAGATAATCACAAGGAACACTGCGCTTGGGGCATGACT
GCCCTCAGCAACCCTTCTGGCGGCAGACACAGTTGTTAGTTTTCCAACATCCTGCTTTCATGAGAA
CAGTTTTCTGTTTGCTCATATAGCCTTCAGTGGTATACTGAGTTGGTCACGACCTTCATTCTTTCGG
CCTGTAACATCTCCCCATTTTTGTTTTTGCATTAATTGAATAAAGGTAATTGCAGGTTGTGCAGCTC
TCAATTGCCGTTTGGTGGTCCAGCTGATTTTGCAGACTTATATCAGCTGTCAGCAGACTCGTCGCA
GGGTTTCTCATTCTCGTTCTTCTTGTCAGTGTCAGTTTCTCTGCTCCAGCAGACCTTCACTCACGTCC
CTGTCCTAGGTGCCAGTTGTCGCTGTTGGTTGTTATGGGAGTGAACGAAGGGGGATGAATGCAGA
ACGAAGACAAAGACAAAAAGTATTTTTGGAAGAAAGGGGTCAGGGGGCTCCTTCTAGTGAACAAG
GGGCCCCCCGCGTACCCTTGNCCGGCGGGCCNTTCTAGAACTAGGGGGATCCCCCCNGGGCCTGG
CAGGGAATTTCNAATATTAAAAGCTTTATTTGATACCCNNTCCGANCCNTTGAANGGGGGGGGG

| | |
|---|---|
| Sequence 496 | cMhvSC038a11 |

CCGCGGTGGCGGCCGCCCGGGCAGGTACGCGGGGAGGTGGTGGCGAANCGCTCCTNCGAAAGGTT
TCNGAAGCTGGTGGTAGCTAGNNAAGATAACGCTGCGTTAGGGNATANNGCTTTTTNATGATGGA
ACTCCGATTGAAAGCAAGTT

| | |
|---|---|
| Sequence 497 | cMhvSC038c06 |

GGGCGAATTGGAGCTCCCCGCGGTGGCGGTCGCAGAAGAGAATCCCGTTGGTCTTGCTGTGCTGG
ATGAAGAAAAGGAAGGGGTGGTCGGCGCAGAAGCGGGGGACGAANNANGGCACACCGCATCACC
ANAANANCAGTTTTTNNNNNTGCAGCCTCCGNGCCTTCCTCATTGACCTCCACAAAAGACTTGNGC
NANAAACCTTTGGAANANNAAANNAGNTCTTGCNTGGNACCANTTCCANTANAATTTCTTGCCCTT
TGCCCA

| | |
|---|---|
| Sequence 498 | cMhvSC004e10 |

NTTCCTCCCACCCTTAGGGGGAAAA

| | |
|---|---|
| Sequence 499 | cMhvSC004e11 |

ACCGCGGTGGCGGCCGAGGTACCTGTCTTGGCCTCCTACAGNCCTTTTTACTTATTTTGTTTTTTAN
AATAGAGACAGGGTCTTACTATGTTGCTCAGACNGGTTNCAAACTCCTAGGNTCAAGCANTCTTCC
AGCCTCAGCCTCTAAAGTGCTGGGATNACAGGCATGAGCCACCACACCCGGCCAAG

| | |
|---|---|
| Sequence 500 | cMhvSC004g06 |

ACCGCGGTGGCGGCCGAGGTNCTTTTTTTTTTTTT

| | |
|---|---|
| Sequence 501 | cMhvSC008b01 |

CCGGGCAGGTGTGCGTGTGTGGAGTAAAATGCATCGGACAGTGATTGACTCCACTTTTGANTGAG
ATGTGGAGGCGGTANTGG

| | |
|---|---|
| Sequence 502 | cMhvSC012d02 |

AGGTACACACAGTTNACCACAAAACAGGCCTNTNTGAAAAAGCCATTGCCATGGACTGCCATACA
GACAATGACAAGACACAAATA

| | |
|---|---|
| Sequence 503 | cMhvSC012d06 |

ACNNGCCAGGNNCNTNNNNGCCTATTACACCTACNTGNCTCTGGNCTTTTATTTGNACNNCGANG
ANGTGGATCTNGAAGGNGNGANCCANTNCTTGCGNNAANTGNCNCATGAGAATCTCGA

| | |
|---|---|
| Sequence 504 | cMhvSC037g12 |

CTCCCCGCGGTGGCGGCCCGCCCGGGCAGGTACCACCATGCCTGGCTAATTTTTATATTTTTAGTA
GAGACGGGGTTTTGCCATGTTGGCCGACTGATATCGACCTCCTGACCTCAGGTGATCTGCCCGCCT
CGGCCTCCCAAAGTGCTGGGATTACAGGCGTGAGCCACTGCGCCTGGCCAAGATTAGAGGTTTTAT
ACTTTGTATCATCCAACTTTGAAATTCTTGCTTGCTGGCACCTTGGCAAACCTACTGCCTGACACAT
GTGAGTGGGTTTCTAAAAATTTTTGT

| | |
|---|---|
| Sequence 505 | cMhvSC038a01 |

ATAGGGCGAATTGGNNCTCCCCGCGGTGGCGGCCGCCCGGGCAGGTACTAGCTACTCTGGAGGCT
GAGGCAGGAGAATGGCGTGAACCCGGGAGGCAGAGGTTGCAGTGAGCTGAGATCACACCACTGC
ACTCCAGCCTGGGCGACAGAGAGAGGCTCCCTCTCAAAAAACGAAACAATGTTCTTGGCTGGGCG
CCAACANNTTTANNACCTGTTAATTCCCAAGCNGGTACCCT

| | |
|---|---|
| Sequence 506 | cMhvSC040g02 |

CTCCCCGCGGTGGCGGCCGCCC

| | |
|---|---|
| Sequence 507 | cMhvSH037e08a1 |

AGGNACTNTNNTTTTTTTTTTTTTTTTTTTCCTGANATGCGNGTGNCCTATNAACTTTCGATGGTAGT
CTCCGTGCCTACCATGGTGACCACGGGTGACGGTGGAATCAGAGGTTNTANNNCNGAGAGGGAGC
CTGAGAAACGGCTACCACATNCAAGGAAGGCAGCAGGCGCGCAAATTACCCANTCCCGACCCGGG
GAGGTAGTGACCNAAAAAAAAAAAANGNANGGANAANACAAGGGTNCCTCGGCCCGCTCTAGAA
ACTAAGNTGGGATCCCCCGGGCTGCAAGGGAAATTTTCGAATATTCAAAGGCTTTNTTCGGATNAC
CCGGNTCGGACCCTTNNAGGGGGGGGGGGGCCCCGGGNTNCCCCCNAGGCCTTTTTTTGGGNTTC
CCCTTTTTTAGNTTGGAGGGGGGGNTT

| | |
|---|---|
| Sequence 508 | cMhvSH066a01a1 |

GCTCCAGCCCCGANCCCTGGACATCTACTCTGCCGTGGATGATGCCTCCCACNAGAAGGAGCTGAT
CGAAGCGCTGCAAGAAGTCTTGAAGAAGCTCAAGAGTAAACGTGCTCCCATCTATGANAAGAAGT
ATGGCCAAGTCCCCATGTGTGACGCCGGTGAGCAGTGTGCAGTGAGGAAAGGGGCAAGGATCGGG
AAGCTGTGTGACTGTCCCCGAGGAACCTCCTGCAATTCCTTCCTCCTGAAGTGCTTATGAAGGGGC
GTCCATTCTCCTCCATACATCCCNATCCCTCTACTTTCCCCAGAGGACCACACCTTCCTTNCCTGGG
AGTTTTGGGCTTAAGCCAANANATAAAAGTTTTTTATTTTTCCTCTTGAAGGGGAAAANGGGCTTC
TTTTTTCCTGGGTTGTTTTNCAAAAAANTTAAAAGNAANCCCCTTTTTNGGATTGTTTTNCTTGGGG
GGAAAAAAAAAAAAAAAGCCCTTTTGNTANNGGGGGGNTNAAAAAACCNGTNNNTNGAAAAANG
TTTTTTTTTTTNTTTTTTTTTTTNGGNTTGGNNNAANANCNTTTTTNTTTNAANCTTTNCCGNGGGGG
GGGGNNNNTTTTTTTAAAAAAAANANGGGGCNCCCCCCNCCNGGGNNGNNGGGGANGAAAATNN
NTANTTNTNGNGTTTTTNTTCCCCCCCCCCCCCNCCCN

| | |
|---|---|
| Sequence 509 | cMhvSH026b01a1 |

TAAAACTTTATTAANAGAATNTTATCAGTCAAATTTCCAGATTAAGAATAACGTTCTTGGTTTCAG
TCTTCATTTGTCTTGCTTGAAACCTATGGTTGCGCATCACCTGCTTCCAGCACTTTAGTGAGATCAA
AAGTGGGCATAATACCCTCCCTGACATCAGGACCATNTCCAGGCTCATCCTNTATNTTAAGCAGAG
CCAGTTCCTGTTGAAAAGCTTCCATGTCAGGCCCTTGAAAAGCAGGCNCTGCTTGATTTTCAATCT
CCCCACTAGGGGCAATACCCGGATTNTNAGTGGGGGGTNCCTTTTTTNGNCGTTTTTNNCTNAGGG
GGGCNCGGGGCANTTCCNNATCCCCCCCGGGGGNGGGNAAAAACNTTNGGGGAANTTTNNNTNTT
TTTTNAAGNNNGNNNGGGNAAATTTTTTTTTTAAAAAAAGNCNNNNNTTTTTTTTTTCCCCCCCCG
GGGNTTTTTTTTTTTNGGGGNNGGGGGAAAAAAANAAAAAAAAAGGNNNGGGGGGGNAAAAAAA
ANAAAAAAACTTTTTTTTTTTTTTTTNNAANACNTTTTGNGGGGGAGCCCCCCCCNTNTTATNTNCT
TNGGNNGGGGGGGGGGNTTTTAAAAAAANAAANNAANCCCCCCCC

| | |
|---|---|
| Sequence 510 | cMhvSH109g02a1 |

ATATAGGGCGAATTGGACTCCACCGCGGTGGCGGCCGAGGTACGCGGGGACGGAGGGCGGTGCCC
GCGTCAGTGACCGAAGGAAGAGACCAAGATGAATACAGAGCCCGAGAGGAAGTTTGGCGTGGTG
GTGGTTGGTGTTGGCCGAGCCGGCTCCGTGCGGATGAGGGACTTGCGGAATCCACACCCTTCCTCA
GCGTTCCTGAACCTGATTGGCTTCGTGTCGAGAAGGGAGCTCGGGAGCATTGATGGAGTCCAGCA
AGATTTCTTTGGAGGATGCTCTTTCCAGCCAAGAGGTGGGAGGTTCGCCTATATCTGCAGTGGAAG
AGCTTCCAGCCATGAGGGACTAACATCAGGCAAGTTCCTTTAATGCCTGGCAAAGCACGTTNCTTG
TTGGGAATACCCCATGACACTTGTCATTTGGGCCGGCCCGCTTCTAGNAACTAGTTGGGATCCCCC
CGGGGCTTGCAGGGAATTTCGAATNTCAAANCTTTATCNGAATTACCCCGNTCTGAACCTTCGAAG
GGGGGGGGCCCCCGNGTACCCCCAANCCTTTTTTGGTTTCCC

| | |
|---|---|
| Sequence 511 | cMhvSH124f02a1 |

AGGACATAGCCCCAGAAGGGCGGACTGGCCGGAGTCCAGGGATGGCAGCCAACGCCCCATAACA
GAGATCAGCATTGGACTACAAGAAGAGGCAAGGAGAAATCAAGGATCAAAATTTAAGTAAAAGA
AAAGTCAAGTCATTAAAAATAGCCCCCTCATTGAAGAGTGGGAACGTAGGTGTGATGTTCTGGCA
TAAGGAGTGAAAAAAGAAAAAGCTCTATTACTTGAAGCTTTTCACCAGGGGCAGAGAGAATGGCC
GGAAGTGAGAAACGTGTGTGTGGATGCTTACACCGATGCCGTCTCCTAATATTGGAACATGGCTTC
CAGAAAGGAGAACCAATTATTCCTAATTCCACGGGCGGCATCCTCTGACTCCCAAACTCCCAAAGT
GGAGGGCAAGAGCTGCCCTTACCTTGAGGAAGCTTCAGAGTGTTTNTGGTAAAACTNTTTCCGGGG
GTGCGACATANGGATNCTTTTCANAGCTCCCTTGGACAATGGTNCCCTTGCCCCGGGGCGGGCCCG
NTTCTAAGAAACTAGTGGGATTCCCCCCCNGGCTTGGANGGAATTTCCATNTTCCAAGCTTTTTTC
GAATACCCGTCCCNAANCCTCNGAAGGGGGGGGGGC

| | |
|---|---|
| Sequence 512 | cMhvSH110a07a1 |

GAATTGGAGCTCCCCGCGGTGGCGGCCGAGGTACTTTTTTTTTTTTTTTTTTTTTTTTTTGGTTTTTTT
TTTTTTTTTTTTTTTTTTTTTTTCCTTTGGGCAACACTTTATTGGGAAANATTTACNCNCGGGGACCT
NTCNTAGGCCAAGCGATNAAAANAGGGCCCCAGGAGCCCTGGGGTCCCNAGGNGGCTCAAATGG
AANCCATGGGACGGCCNNTNTAAAACTAGNGGATCCC

| | |
|---|---|
| Sequence 513 | cMhvSH038g10a1 |

AGGTACGCGGGAGGGTTCTGGTGTTTGGTTTCTTCATTCTTTACTGCACTCAGATTTAAGCCTTACA
AAGGGAAAGCCTCTGGCCGTCACGCGTAGGACGCATGAAGGTCACTCGTGGTGAGGCTGACATGC
TCACACATTACAACAGTAGAGAGGGAAAATCCTAAGACAGAGGAACTCCAGAGATGAGTGTCTGG
AGCGCTTCAGTTCAGCTTTAAAGGCCAGGNACGGGCCACACGTGGCTTGGCGGCCTCGTTCCAAGT
GGCGGCACGTCCTTGGGCCGTCTCTAAATGTCTGCAGCTNAAGGGCTTGGCACTTTTTTTAAATAT
AAAAAATGGGGTGTGTATTTTTTAATTTTTTTTTGTTAAAGTTGATATTTTGGGGTCTTCTGTTGGA
CAATTCGGGGGGTGGATCCTGTTCTGCGCTGTGTACCTGCCCGGGGGCGGGCCCGCCTTNTAGGAA
ACTTAGGTGGGATCCCCCCCGGGGGCTTGCCAGGGGAAATTTNNGATNNTCANAGGCTTTATTNCG
AATACCCGTTCCGAACCCTTCNGAANGGGGGGGGGGGCCCCGGGGTACCCCCAANCTTTTTTGGTT
TCCCCCTTTTAAAGTGGAGGGG

| | |
|---|---|
| Sequence 514 | cMhvSH109f02a1 |

CCGGGCAGGTACTTGGAAAACTTGTTGAAGATGATGGGGNNGGGAAGGGCCACCANAAAAANAA
NANNTTNTTNTTCTTNTGCTGGCGATGAGCTTTCCCGCCAAGGTGACCGGGTGGGTGTCTCCATAG
CCCACAGTTGTCATGCTGATGGTGGCCCACCACCAGCAGATGGGGATGCTGGTGAGGCTGGATGT
GTGGTCATCTTTCTCCACNGAGTNGATAAGCACAGAGAAAATGGAAATGCCCACAGAGAGGGAAG
AGAAGCCAGGAAGCCCAACTTTCATGGTTAGCNTGTGTCTCCAGTGTGGCCACCNTAGAAGACCC
GGAAAGTCCTACCCGAGTGCCCGGGCCAAGCCTTTTAGAATTCCGGGAAAATCCTCATTAAAGCCC
GTTAGGGATCTGNGACCCACCCTTGCCCCATGTTCTCNAATATCCCTCACTCTCTTTNCTCCTTGGG
TGGTCTTACAGCCCCAACNGTTGGCATAGGAAAGGGGAAATAATTAGAAGACAAAAGTTCAAATG
GATGGTTCNAACAGGGTTTTTTTTCCCANGAAATTTTCTTTTTGGACAAAGGGAAGCCGGGCCAAG
CCCAAGGCCCCGGGACCGGGCAAAGCCTCCC

| | |
|---|---|
| Sequence 515 | cMhvSH110d10a1 |

ACGCGGGGATACAAGAAAGAGGAAGAGAAGCAGGAAGATTCTACATACAGGCTGGCTGTGTTTCC
CCTGGGGCATGCTCCTGTTTACTGGTCCCATGCCAGGTTGACTCATTGCCTCGTTCATGGGTGGAAT
TAAAATGCCTACCTGGGGAATAAATAGAGCAAGGCTGGGTGCTCACCTCCACAGCGGCTTCCTTG
ATCCTTGCCACCCGCGACTGAACACCGACAGCAGCAGCCTCACCATGAAGTTGCTGATGGTCCTCA
TGCTGGCGGNCCTCTCCCAGCACTGCTACGCAGGCTCTGGCTGCCCCTTATTGGAGAATGTGATTT
CCAAGACAATCAATCCACAAGTGTCTAAGACTGAATACAAAGAACTTCTTCAAGAGTTCATAGAC
GACAATGCCACTACAAATGCCATAGATGAATTGAAGGAATGTTTTCTTAACCAAACGGGATGAAA
CTCTGAGCAATGTTTGAGGNGTTTATGCAATTAAATATATGACAAGCAGTCTTTGGGATTTTATTTT
AACTTTTCTGCAAGACCTTTTGGCTTCACAGAAACTGGCAGGGNNTTGGGNGGAGNAAACCAACT
ACCGGATTTGNTTGCAAAACCCACACCCTTTCTCTTTTCTTTANNGGCCTTTTTGACCTACNAAAAC
TTACAANGAANAAANTTGNTGGAAAACCTNGCTTTNCATGGTTTTATTTTAAATTAAAATTGGANG
GGCAAAAAAAAAAAAAAAAAAAAAAA

| | |
|---|---|
| Sequence 516 | cMhvSH046b03a1 |

CCGAGGGTACTTTTCTGAGACTTNATCCTCGAGGCCTGGTGGGCTACCGGCTCTTTTCATCTTCACG
GCCACCCACAGAAATGAAGCAGAGTGGCCTAGGCTCACAGTGCACAGGGCTGTTCAGCACCACAG
TGCTGGGTGGCTCCTCCAGTGCCCCGAATCTTCAGGACTACGCCCGCAGCCATGGCAAAAAGCTAC
CACCTGCCAGTCTGAAGCACCGAGATGGGTTTGAAGGGTGTTCCATGGTGCCTACCATCTACCCTC
TGGAAACACTGCATAATGCCCTTTCCCTACGTCAAGTGAGTGAATTCTTGAGTAGAGTCTGCCAGC
GCCACACTGATGCCCAGGCACAGGCATCTGCAGCCCTCTTTGATTCCATGCACAGCAGCCAGGCCT
CAGATAACCCATTTTCTCCACCACGT

| | |
|---|---|
| Sequence 517 | cMhvSH108g03a1 |

AGGTACTTTTTTTTTTTTTTTTTTTTTTTTTTTGGTTGNGTAATNCTTTATTTGAAAAAATGAAAAGT
GCACACACACACACACACATACACACACACACACACACACTTACATAGGCACAGGATAATCTGGA
AGTATGACCAGCAAATGATAACTGATTCCCTNAGGGGANAANAAACTGGGTGGCTGAAGGACAG
GAATGAGAAANAAGGACAGTTGCGCTTGTTTGTATCGTTTGAAATTGTCCAGTGTGTATGTGTTCT
TTTCAAATGTTTGAAGAACCATTGGCTCCCTTATCAAAATGTAAATACCAAGGAAAATN

| | |
|---|---|
| Sequence 518 | cMhvSH075f10a1 |

GNGGNGGCGGCCGCCCGGGCAGGTACGCGGGGGGCGGCGGCGGAGAGAGCTGGCTCAGGGCGTC
CGCTAGGCTCGGACGACCTGCTGAGCCTCCCAAACCGCTTCCATAAGGCTTTGCCTTTCCAACTTC
AGCTACAGTGTTAGCTAAGTTTGGAAAGAAGGAAAAAAGAAAATCCCTGGGCCCCTTTTCTTTTGT
TCTTTGCCAAAGTCGTCGTTGTAGTCTTTTTGCCCAAGGCTGTTGTGTTTTTAGAGGTCCTATCTCC
AGTTCCTTGCACTCCTGTTAACAAGCACCTCAGCGAGAGCAGCAGCAGCGATAGCAGCCCGCANA
AGAGCCAGCGGGGTCGCGTAGTGTCATGACCAGGGCGGGAGATCACAACCGCCAGAGAGGATGC
TGTGGATCCTTGGCCGACTACCTGACCTCTGCAAAATTCCTTCTCTACCTTGGTCATTCTCTCTCTA
CTTGGGGAGATCGGATGTGGCACTTTGCGGTGTCTGTGTTTCTGGTAGAGCTTCTATGGAAACAGC
CTTCTTTTGACAGCANTCTACCGGGCTGGTGGTGGCANGGGTNTGTTTTGGTCCTGGGAGCCATNA
TCGGGNGACTGGGNGGGACAAGAATGNTNTAATTNAGGGNGGCCCCACCCTNGGGTGGNGGGTA
CCNCGGGCCCCATATAAAAANAAANGGNNANCCCCCCGNGGGGGGGGGAAANTTTAAATCNANG
NCTTTCCCNCCCCCCCCCCCCNGGGGG

| | |
|---|---|
| Sequence 519 | cMhvSH128d09a1 |

CCGGGCAGGTACGCGGGAGGCATGCACCACCACGCTCGACTAATTTTTGTATTTTTAGTAGAGACG
GGGTATCACTATATTGGTCAGGCTGGTCTTGAACTCCTGACCTCAGGCGATCTACCCGCCTNGACC
TCCCAAAGTGCTGGGACTACAGGTGCCCACCACCACGCTTGGCTTATTTTTTTTGTATTTTTAGGAG
AGACGGGGTTTNACCGCATTAGCGAAGATGGTCTCGATCTCCTGACCTCGTGATCCACCCGCCTCG
GCCTCCCAAAGTTCTGGGATTACAGGCTTGAGCCACTGCGCCNGGCCTAGAACCCTGCTTCTCATA
TAAGATGGGCCTGCACCTACCTCTGGCATGTTTTTCTTTGTGTATTTCCCGTTTTTNATCCTGTAACT
AAATGCTCATTATTTAANAACACTCCAGTTACTTTTCCCTTTAGGCCTGGCAAAACTTTNCTNTTTC
TTTTTTTTTTTTTTTTATAAACTGNAACCTTTGGGGCGGGTTTTAGAAAACTAANTGGGATCCCCCC
NGGGNCTNGAAGGGGAAATTTGGNNTTTTCAAANCTTTAATTNNANTACCCGNCCCANCCCCCAA
GGGGGGGGG

| | |
|---|---|
| Sequence 520 | cMhvSH075h11a1 |

TGNACCTCCACCGCGGTGGCGGCCGTTTGAGAAGCCANCGCTCACCCACCCGGGGTCTCTGTGCAT
TGACCTTTGGGTGCTGACTTGGAGAAAAGCACAAACACGACCAGTCCCATCCTGGCTCCCGTGGG
GCTTCTTCTATCTACGCATTGTATCGACTGCATTAGTTGGACTAAGATGATGACTCAGTTAAAGGA
GGAGACAAATGCTGACTGTCTAAGCAAGAATGGCCCAAGCTGGCAAGAAAAAGCACACTGNGAT
ACATAGGGATACAGGAAGGGCAGGAGCCTTTTTGCCTGCCGGGATCTAACAANCATTTACATTTTG
TTTTGCCTGCCAAACCTATCAAGAAGGGATTTCTTGTTTGGGCCCAGGGGGAGTCTCCACTTGGAA
ACAAAACAAAAAATGGCANGTCAAAAAAGTTCTTTGAGGTGTCCCTATTCCAAGCCAGCCCAAGA
AGTCCTCAATCCCGTCATCCCACGGGGAAGAAGTTCCTTTTGAAGGGGAAAGCATGAAAAGTTCC
AGCCTCATGGCCTCTTGCCTTATTGGGTCAATTTTCTTCGGGGAATCACTTGTGAATCAATGAATAT
CTTTCATTTACCTCTGCCGGGACCCACCCCATGGTTTCAAGGGGNGGCNTT

| | |
|---|---|
| Sequence 521 | cMhvSH105f02a1 |

TGTACTAANCCATTGTGACAGAAACTTNCTTTACCATTGATGAGCTGGAAGAACTTTATGCTCTTTT
NNAAGGCANTAACATCTCACCAGCTGNTACTGGGGCGGGAGCAGCNACNCGCTGGACCGGCATGA
CCCCAGCCTGCCCTACCTGGAACAGTATCGCATTGACTTCGAGCAGTTCAAGGGAATGTTTGCTCT
TCNTCTTTCCTTGGCGCATGTAGGAACTCACTCNTGACCGTTTCANGGCCTTCCGCTTTGTTCCAGT
TTTATTTAGGATNAAAAATNGGAGGACCTCTTTTGGATT

| | |
|---|---|
| Sequence 522 | cMhvSH007h11 |

AGGTACCCGGGATTTNACCANTGTNACTGTGCTAAATGGTTCTGTCTTCCTCAGTGTGATGGAGAA
AGCCCAGAAAATGAATGATACTATATTTGGNTTCNCANTGGAGGANCGCTCATGGGGGCCCTATA
TCANCNGTATTCAGGNNNTATNNGCNAACANTNATNACCNANCCTACTGGNAACTTANGAGTGGA
TTGCCTNNCCCTGGTNCACGCACTGGTAGTCTACGNTGTCCGCAATGGNTNAAAACTTGGAAGTCT
CTTGAGCCCAGGAGGCNATAAAGTCCCAANACTTNCCTNATCTGCCNANTTATACCTTNATGCCTG
GGGCAACACAACNAGACNTGCCNNCTNAAAAAAA

| | |
|---|---|
| Sequence 523 | cMhvSH016e11 |

CCGGGCAGGTACAACACTCTGTCCCTACAAGGGCACAGGTGCCACCTTGAGCAGCTGTGACTATGT
CTAAGGCCATCCGGTTTTGCATCACCACCTTCCTGATCTGATCAAACTCATCAATTAACAAAAGGA
GGGCAGCTCAGGTGTAATTCATGGGCCCAATCTCTGTGTTCTGCAAGGGCTGTAACCTGCATTTCT
ACAGTGATGACACCTGTTCCAGGGACAGTTATTGCTAAGGGGTAGAACCACTAGGGGCTCAATGC
ACTNACAAAAACTGGGAACACAGC

| | |
|---|---|
| Sequence 524 | cMhvSH025e11 |

GGGGCAGGCTTGCCATGGGTTTTGNGACACCCCNATCCAAAGCTCACCATGTTGCATCCCGCCCAT
TGNCTGTGGGACCCCAAGTTTCTAGCCATGTCCAGTTCTTCACAAAAGCTGGATGCACATGCCAAG
GCAAGCCATCCACAGCTGCTGCTGGAAGGGTGGTGCAGATCTAACAGTTGGAGACATTGGCCACC
TCAGCATAGGTGTGAGCCCANTCCACAATGTTGTTGGAGCATGCCAACCTGTGGCTGAGCAAATA
ACTCCCAAGAATTTGGCAGACAATTTTCGGCCCTTGGACCTTGGATTTATTGATGGCCCAACTGCA
CACTGCCAAATGCTGTCACAAGAGGGGCACCACCACTTCTA

| | |
|---|---|
| Sequence 525 | cMhvSH027a12 |

AGGTACGCGGGGAAGCGCAAAAGAAGAAAGATGAGGCAGAGGTCCAAGTAAACCGCTAGCTTGT
TGCACCGTGGAGGCCACAGGAGCAGAAACATGGAATGCCAGACGCTGGGGATGCTGGTACCCGTG
CCCAGGAGGACGCCGAGCTCCAGCCCCGAGCCCTGGACATCTACTCTGCCGTGGATGATGCCTCCC
ACGAGAAGGAGCTGATCGAAGCGCTGCAAGAAGTCTTGAAGAAGCTCAAGAGTAAACGTGTTCCC
ATCTATGAGAAGAAGTATGGCCAAGTCCCCATGTGTGACGCCGGTGAGCAGTGTGCAGTGAGGAA
AGGGGCAAGGATCGGGAAGCTGTGTGACTGTCCCCGAGGAACCTCCTGCAATTCCTTCCTCCTGAA
GTGCTTATGAAGGGGCGTCCATTCTCCTNCATACATCCCCATCCCTTTACTTTCCCAAGANGACCAC
ACCCTTCCTCCCTGGAGTTTTGGCTTAAGCAACAAGANAAAAGTTTTTATTTTTTCTNTTGAANGGG
AAAGGGCTTCTTTTTTCCTTGCTTGTTTTCAAAAATTTAAAAAGG

| | |
|---|---|
| Sequence 526 | cMhvSH029e11 |

AGCTCCACCGCGGTGGCGGCCGAGCTGACGCAAACATGCAGATCTTTGTGAAGACCCTNGNNGGN
NGNNACCATCACCCNNANAAGAAAATCCTTTTGACNCCATTGAGAATGTCAAAGCCAAAATTCAA
GACAAGGAGGGTATCCCACCTGACCAGCAGCGTCTGATATTTGCCGGCAAACAGCTGGAGGATGG
CCGCACTCTCTCAGACTACAACATCCAGAAAGAGTCCACCCTGCACCTGGTGTTGCGCCTGCGAGG
TTGGCATTATTGNAGCCTTCTTCCCCCGCCAGCTTGCCCAGNAAATACAAACTGCGAACAAGTATG
ATTCTGCCGCAAAGTGGCTATTGCTNCGCCTTCACCCTNGTGCCTGTTCAACTGCCCGNAAGGAAA
GCAAAGTTGTTGGTTCACACCCAAACAAACCCTTGCGGTCCCAAGGNAAGTAAANGNTCANAATT
AAAGGGTTGGCNTCTTTTCCTTTTGAAAGGGGACANNNCNCTTCCTGGCCCNCAGNGCNCCCCGNT
GGGCCCCCTGGGNAACCCTTCCAAATTAAAAANNGGNTTCCCTTTTTTCAATTTTGGACCTTGGGA
AGCCAAGNCTTTCTANATAGAANANATNGTATCNNTCACATATTANNATACGNGNTTNCCCTTNG
GGCCCCGATNTTNTTAANAANACCTNAAGTGGGGATTCCCNCCCGGNGCCTTGCGAAGGGAATTT
CNGAAATATTTNAAAAGCCNTTAANTNNGANTTCCCNGGTCCGAACCCNTCCCGAGGGGGNGGGG

| | |
|---|---|
| Sequence 527 | cMhvSH035c08 |

AGATACGCGGGGGAGGAGTGAGCTCTTGGGGTGTCCAGTTGGTTGCCGCGGCAAGTCTCTCCGAG
CAGCGCATTTGTCTTCTAGGCTGCTTGGTTCGTGCCTCCGAGAAAGGGGTCTNCTGCTGCCAGCTA
AGTGTGGGAGAACTTGTGCACGTATCTCCCCTCCGAATCCCAACGATGGGTAACGCCAGCTTTGGC
TCCAAGGAACAGAAGCTGCTGAAGCGGATGCGGCTTCTGCCCGCCCTGCTTATCCTCCGCGCCTTC
AAGCCCCACAGGAAGATCAGAGATTACCGNGTNGTGGTAGTNGGCACCGCTGGTTGTNGGTGAAA
AANTANCTGCNCNTNGGCCGGNCGNTTCTANAACTANTGGANNCCCNGNGCTGCATGAATTCNAT
ATCNAAGCNTTATTTNATTCCCGTCGACCNTNTNTAGGGNGGGGGGACCCGGATNCCCCCAANAA
TTTTTGTTTCCCCTTTTNATTNNAGGGGNTTTAATATNCACCTCCTATNGGGCNNCTNANTCNTTNG
TNCAATTTNCTTGNTCCTCCTCGTTGNTNAAAAATNTTGGATATTATTGTTTCCCCCCCCTNTATGA
NANCNNACNAAAANNNANTNANTTTAANTANTTTTTTTTTTTTTTTTTCCCC

| | |
|---|---|
| Sequence 528 | cMhvSH041f10 |

AGGTACGTCCAAATGACGAAGTCACTGCAGNGCTTGCAGTTCAAACAGAATTGAAAGAATGCATG
GTGGTTAAAACTTACCTCATTAGCAGCATCCCTCTACAAGGTGCATTTAACTATAAGTATACTGCC
TGCCTATGTGACGACAATCCAAAAACCTTCTACTGGGACTTTTACACCAACAGAACTGTGCAAATT
GCAGCCGTCGTTGATGTTATTCGGGGAATTAGGCATCTGCCCTGATGATGCTGCTGTAATCCCCAT
CAAAAACAACCCGGTTTTATACTATTGGAAATCCTAAAGGTAGGAATAATGGGAAGCCCTGTCTTG
TTTTGCCACACCCAGGNTGATTTCCTCTAAAGAAACTTGGCTGGGAATTTCTGCTGNGGGTCTATA
AAAATAAAACCTTTCTTTAACCATGGCTTTCTTCCAAAAANNAAAAATTGTAATNNTANATAAAAA
TAATGGGGNCCCTTGGGCCGCTTCNTANNAAACTTAAGGTGGGGATCCCCCCCC

| | |
|---|---|
| Sequence 529 | cMhvSH044f03 |

AGGTACGTCCAAATGACGAAGTCACTGCAGTGCTTGCAGTTCAAACAGAATTGAAAGAATGCATG
GTGGTTAAAACTTACCTCATTAGCAGCATCCCTCTACAAGGTGCATTTAACTATAAGTATACTGCC
TGCCTATGTGACGACAATCCAAAAACCTTCTACTGGGACTTTTACACCAACAGAACTGTGCAAATT
GCAGCCGTCGTTGATGTTATTCGGGAATTAGGCATCTGCCCTGATGATGCTGCTGTAAATCCCCAT
CAAAAACAACCGGTTTTATACCTATTTGAAATCCTAAAGGGTAGNAATAATGGGAAGCCCTGGTCT
GTTTTGCCACACCCCAGGTGGATTTTCCTCTAAAGGAAACTTGGCTGGGAATTTCTGCTGTGGTCTA
TTAAAAAATAAAACTTCTTAACATGCTTTCTCCNAAANAAAAAAAGAGGNNAAAAAATATACAAA
GGGTTACCTTNGGGCCGGNTNTTAANAAACTAAGNGGGAATCCCCCGGGGCCTTGGCAAGGGAAA
TTTCCGATNNTTCCAAAGGCTTTATTCCGAATACCCCGGTTCGGAACCCCTTTCGNAGGGGGGG

| | |
|---|---|
| Sequence 530 | cMhvSH053f04 |

TCCACCGCGGTGGCGGCCGCCCGGGCAGGTACTCGGGGAGGCTCCTGGGGTGGNNTCCAAATCAC
TCATTTGTTTGTGAAAGCTGAGCTCACAGCAAAACAAGCCACCATGAAGCTGTCGGNGTGTCTCCT
GCTGGTCACGCTGGCCCTCTGCTGCTACCAGGCCAATGCCGAGTTCTGTCCAGCTCTTGTTTCTGAG
CTGTTAGACTTCTTCTTCATTAGTGAACCTCTGTTTCAAGTTAAAGTCTTGCCAAAATTTTGATTGC
CCCTTCCCGGGAAGCTGTTGCCNGCCAAGTTTAGGGAGTTGGAAAGAAGATTGCACGGGATCAAG
ATTGTCCCTTTCANGAAANNGAAGGCCTCATTTTGGCCGGGAAGTTCCTTGGGTNGAAAAAATNAT
TTTGAAANGAAAAATGGTTAAGNTNGTTGNTGGNACCAATTGGTTAAAAAAANACCTTTTTCCAAT
CCCCCTNGGGTTTTTCNCAACTTGGNTCCTTTTTCAAATTNGAACAACCCCCTTGGATTCCTTTCAA
CCTTGGCCANGAAAAATGGTTNAANAAGGGGTTTTTCCAAANCGGTTCTTTTGGCTTTTTAAAATT
AAAAANTCCACCTTTNGGCCTTCTTTCCCCCNAGATGAANTATGGAAACAANNAAGAAAATTTACT
NTNTTNTNTANNAANGNNGGTTTCCCCTTNTGGGTCCCGNTTTNTTANANGAANCNTTANTGTTGG
GANTCCCCCCCCCNGGGGCNTTGGNNAAGGGNAAATTTNTNGAATATTNCAAAGGCTTTTATTCCG
ANTANCCNGGGCNCTACCCCTTCAANGGG

| | |
|---|---|
| Sequence 531 | cMhvSH054g02 |

AGGTACAAACCCAGTTTGTTTTCAAAAAATCACAGNGNGCAATGCAACTCATNACTNTATAAAAG
CAAGCTTAGGCTACCTGAAAGATTTTCCCTTGGAAGTTTAGCGTATGTTTGACTAACAAGAATTCC
CTACATCAGAGACTCTAGGTGCTATATAATCCAAAAACTTTTCAGCCTGTTGCTCATTCTGTCCCAT
GCTGGCAATAATACCTTGTCAGCCCTTTACCCTTATTTTGGAATTGCTCCATCTCCTGGTGGGGACT
TGGTATCTTGTCTGCCATATCAGNAACACAATACCCCTGAAGGAGGTTCTGATTTGATTTTTTTTTT
TTTCTTCATGCCTACCCTTTTTTTGGGAAGTTTTCCAGCNCGCCAATTTTTGAAAATTGAAAAATTG
ACAAAGGGTGGTANTATTTTGGNTTCCAAATTTTGTCAATTTTCCCCAACCCATTTGGCAATTTTAC
CAAACCCTTTCTTTAAACCTTTAAAAATGGGGGGTTAAACCCCCCTTAAAGGGCCAATTNTTTCAA
AAAANNAAAGGCNANGAACNTTGGCCANTTGAAATTANAAAACCGGGGAAAANTNTGAAAAAAA
AAAAANGGAAACCCTNANCCATTTTTTATTTTTTTGNCNTTTTTAAAGCCANTTCCCTTTNACTTTT
TTNAACCCCTTTTTTATTGAAGAAATTTGGAAGAAGTNGGGAACNTTTACAATTTTTCCCNTTTTTT
TTTAACCATTTTTTTCCGNAATACCTTANNTTTTTTTT

| | |
|---|---|
| Sequence 532 | cMhvSH054h05 |

CCGGGCAGGTACCCGTGCCCAGGAGGNCGCNGAGNNCCAGCCCCGAGCCNTGTTNNTTTTACTCT
GCCGTGGATGATGCCTCCCACGAGAAGGAGCTGATCGAAGCGCTGCAAGAAGTCTTGAAGAAGCT
CAAGAGTAAACGTGTTCCCATCTATGAGAAGAAGTATGGCCAAGTCCCCATGTGTGACGCCGGTG
AGCAGTGTGCAGTGAGGAAAGGGGCAAGGGATCGGGAAGCCTGTGTGACTGTCCCNCGAGGAAC
CTCCTGCAATTCCTTCCTCCTGAAGTGCTTATGAAAGGGGCGTCCCATTTCTCCTCCATACCATCCC
CATCCCTCTTACTTTCCCCAGTAGGGACCCACACCCTTCCTCCCTGGGAGTTTTGGCTTTAAAGNCA
ACAAGATNAAGGTTTTTTATTTTTCCTCTGAAAGGGGAAAGGGCTTCTTTTTNCCTGCTGGTTTTCA
AAAAAAATTAAAAANG

| | |
|---|---|
| Sequence 533 | cMhvSH055b06 |

GGAGCTCCACCGAGGTGGCGGCCGAGGTACGCGGGAACATCAAACTGTTAATCGAATGCAGGCTC
CAGGGAGAAGCAACTTCCTGGGTATGCGTGTTAAGAGACAAAAAATGATGACGTTTGATGACCAC
TCCACCAGAAAAGGGAAGAAAGCCTGAGGGGACTACGTGGACCTCCCTAAACACACTGCGCATGC
TCCATTCCAAACGGTATGGCGAGCACTGCGCATGCGGGAAACCCACCCTGTAAGGGAAGAATCCT
GGGAAAGAGGCGAGCCTATGAAGTCCCAGGATCAAGGTTAGAGACCCTTTTTTTACTGTCTTCTTG
TGCTCTCTTTTCTCTCTTGGACCTTCAGGCGCCTGCTTGGGTCTCTTTCAAGCGAATTTTGCTTTCTT
TCCTGNTCTAAAGCCTTTTAACTAAAC

| | |
|---|---|
| Sequence 534 | cMhvSH055b06 |

GGAGCTCCACCGAGGTGGCGGCCGAGGTACGCGGGAACATCAAACTGTTAATCGAATGCAGGCTC
CAGGGAGAAGCAACTTCCTGGGTATGCGTGTTAAGAGACAAAAAATGATGACGTTTGATGACCAC
TCCACCAGAAAAGGGAAGAAAGCCTGAGGGGACTACGTGGACCTCCCTAAACACACTGCGCATGC
TCCATTCCAAACGGTATGGCGAGCACTGCGCATGCGGGAAACCCACCCTGTAAGGGAAGAATCCT
GGGAAAGAGGCGAGCCTATGAAGTCCCAGGATCAAGGTTAGAGACCCTTTTTTTACTGTCTTCTTG
TGCTCTCTTTTCTCTCTTGGACCTTCAGGCGCCTGCTTGGGTCTCTTTCAAGCGAATTTTGCTTTCTT
TCCTGNTCTAAAGCCTTTTAACTAAAC

| | |
|---|---|
| Sequence 535 | cMhvSH058f12 |

GGTGGCGGCCGAGGTACGCGGGGAGGCTCCTGGGGTGGNGTCCAAATCACTCATNGANAAGAGA
AANCTGAGCTCACAGCAAAACAAGCCACCATGAAGCTGNCGGTGTGTCTNCTGCTGGTCACGCTG
GCCCTCTGCTGCTACCAGGCCAATGCCGAGTTCTGCCCAGCTCTTGTTTCTGAGCTGNTAGACTTCT
TCTTCATTAGTGAACCTCTGTTCAAGTTAAGTCTTGCCAAATTTGATGCCCCTCCGGAAGCTGTTGC
ANNCAAGTTAGGAGTGAAGAGATGCACGGATCAGATGTCCNTTNAGAAACGAAGNCTCATTGCGG
ANGTTCCTGGTGAAAATAATTTGAAGAAANNTTTTGTNGAGACCATGTNANNAACTTTTNATCCTG
GTTTCCACTGNNTTTTCAATGACACCCTGATCTTCAACTGNAGNAATGTTAAGGTTTTCAACTGTTN
TTTGNTTTTAATAAAATTCACTTTGCTCTTCCAAAANNNAAATATTTNGTTTTTTTCCCNCCCCTTAC
TTNTAGNGTACCCTGCCCCGGGCCGGGCTCCGNTTTTTAANAACTTAGNGGGGNNTNCCCCCCCGG
GGCCTGCCAGAGGAAATTTTNTATTTTAAAGCCTTTANTCCNTNNCCAGGCNGACCNTNGNGGGG
GGGGGGCC

| | |
|---|---|
| Sequence 536 | cMhvSH058g12 |

CCGGGCAGGTACTCGNGGGGCAAGGTCATCCCTGAGCTGAACGGNAAGCTCACTGGCATGGCCTT
CCGNGTACCCACTGCCAACGNGTNAGNGGTGGACCTNACCTGCCGTNTAGAAAAACCTGCCAAAT
ATGATGACATCAAGAAGGTGGNGAAACANGCGTNNNAGGGCCCACTCAAGGGCATACTGGGCTA
CACTGAGCACCAGGTGGTCTCCTATGACTTNAACAGCGACACCCACTCCTNCACCTTCGACGCTGG
NGCTGGNATTNNCCTNAACNACCACTTTGNCAAGCTCATTTNNTGGTATGACAACGAATTTGNCTA
CATGCAACAGGGTGGTGGACCTGANGGCCCACATGGCCTNCAAGGGAGTAAGACCCCTGGACCAC
NGGCCCCAGCAAGAGCCCANGACGNAGAGAGAGACCCTCACTGCTNNTGAAGGGCGTGCCACAC
TNAGTTCCCCANCAAACTTGAATTNTTNCCNTTCTCACAGTTTGCATGTAAACCCCTTGAAAAGGN
GANGGGTNTAAANGAGCCNTACCTTTNTNATTTTNCCTTTNGGCCGGGTTTTAAAANTAGGTNNGA
TTCCCCCGGGCCTTNGAANGAANTNNTAATTTTCNAACCTTNAACCGAATTCCCGGNTTGNNCCCT
AAAAAGGGGGGGGGG

| | |
|---|---|
| Sequence 537 | cMhvSH060g10 |

AGGTACAAACCCAGTTTGTTTTCAAAAAATCACAGTAGCAATGCAACTCATCACTCTAGAAAAGC
AAGCTTAGGCTACCTGAAAGATTTTCCCTTGGAAGTTTAGCGTATGTTTGACTAACAAGAATTCCC
TACATCAGAGACTCTAGGTGCTATATAATCCAAAAACTTTTCAGCCTGTTGCTCATTCTGTCCCATG
CTGGCAATAATACCTTGNCAGCCCATTACCCTTATTTTGAATTGCTCCATCTCCTGGGTGGGGACTT
GTNATTCTTGGTCTGCCATATCAGGAACACCAAACCCCTGGAAGNAGGTTCTGCATTTGGATTCTT
TTAGGTGGGGNTCTTCCANGGCCTTACCCCTTTTTTTTTGGGAAAGTNTTCCAGGCCCGCCAATTTT
TGGAAAAATGNAAAATGGACCAAGGGNGGTATNTTTTTCGGAATNCAAATTTTTTCCATTTTCCCA
CCCAATTTGGCCATTTACCAAACCCTTNTTAAACTTTAAAAATGGGGGTTAAACCCCTTAAAAGGG
CNATTAATTCAAAAAAGAAAAGGCCAGGGACNTTGCCATTGTAATAAAAAACCGGGGAAANTTAN
GANAAAAAAAAAANGAAAACCCTTACCAATTTTAATTTTTNG

| | |
|---|---|
| Sequence 538 | cMhvSH062c09 |

AGGTACGCGGGATCAATGACATGGTCACGGAAGGCAAGTNGGNTGACTTCAACGGAANNANTATC
TCCTTNCTNAACTGGGACCGTGCACAGCCTAACGGTGGCAAGCCGAGAAAACTGTGTCCTGTTCTC
CCAATCAGCTCAGGGCAAGTGGAGTGATGAGGCCTGTCGCAGCAGCGAAGAGGTACATATGCTNA
GTTCACCATCCCTCAATAGNGTCTTTCTCCAATGTGTCCTCCAAGCAAGATTTCATCATTAACCTTA
TAGGGTTTCATGAANCTCTAAAGGATCAAAGGTTAAAAAATTCATAAAATTTTTTTACTTTTATTTA
AAAAAAAATTTGCCAAACCACCAAAAGGAATCAAATTGGTTCCCANTTAGGCCANAATTAATNGG
AATTAGGCAATTCAAGGCCCAAAATTTTTTTTGGCCTTAAAACCACCAATTTTTTCTTTTTTNGGGG
GAATTTTTTTGGCCCCTTTNCCCTTGGGGGGGTTAAATAAAGGGGGGGAATTNCAAGGAAAAAATT
ATTTTGGAATNCCCNATTGTTGCCCACCCGCCCAGAAATTNAAAAAAATGGGGNCTTTTNTTGNCT
TAAAAACCAAGGACCTAAAAAAAATCCCTTTTNCTTNCTCTTANGNCCCNTTTTCTTCAACCTTTTG
GTNNCCCCTGGCCCCCGGGGCCCGGGNCCCGCCTTCTTAAGAAACCTTNAGGTTNGGGAAATCCCC
CCCCCNGGGNCCTTNGTNANNGGGAAATTTCCCCANANTTCAAAAGGCCTTTAATTCGAAATACCC
CGGTTCNGAACCCCNTCNTAANGGGGGGG

| | |
|---|---|
| Sequence 539 | cMhvSH063c04 |

AGGTACAATCTANTTAAACAAGCAGAATAGCACTAGGCAGAATAAAAAAATTGCACAGACGTATG
CAATTTTCCAAGATAGCATTCTTTAAATTCAGTATNCAGCTTCCAAAGATTGGTATGCCCATAATA
GACTTAAACATATAATGATGGCTAAAAAAAATAAGTATACGAAAATGTAAAAAAGGAAATGTAA
GTCCACTCTCAATCTCATAAAAAGGTGGGGAGTAAGGGATGCTAAAGCAAAATAAATGTAGGTTC
CTTTTTTTCTATTTCCGNATTATCATGGCAGNCTGCTTCTTTTNGATAATGGCCTNAGGGGTTACCC
CCATTTTTAAGNTTTAGGAGGGNTTNGNAAATTGCCAAATGGTNGGGGAAATGAAAAAATTTGGA
ATTCAAAATATTTACCACCCTTTGNTCAATTTTTCCATTTTTCAAAAATTTTGGCCGGGCCTGGGGA
AAAACCTTTTCTNCAAAAAAAAAAAAAGGGGGTTAAGGGGCCAATTGNANCGNAANAATAAACA
CNTNNTATNTTNGTTNCGNAAATTNCATGAAAACNCTTTCTTNTCCAAGGGGGGGNT

| | |
|---|---|
| Sequence 540 | cMhvSH071c06 |

AGGTACGCGGGGCCGGGCGCGGTGGCGCGTGCCTGTAGTCCCACCTCAGCCTCCCATCCTTGTCTA
CCTAATTAGGCTTTGTGTAACTCAGTGTTGCAAAGCTTTTGACATCTGTTTGAGTTAATGTTTATAT
ATGTTGTTACTTAAGGGTTTCACATTAAATTTAAACATACTTATATTTTATAACCAAACAAGTCATA
TTGGGGCATACTCATTAGGATTGAGTGCTTTCTTACACCAAAATACATGTATACAAAAGATTTAAA
ACACTTTTCGGCCCGCTCTTAGAAACTAGTGGGATCCCCCGGGCTGCANGGAATTCNGATATCAAA
GCTTTATCCGAATACCCGTNCGACCCTCGGAGGGGGGGGGGCCCCGGTACCCCAGCCTTTTTGGTT
CCCCTTTTTAGTNGAAGGGGT

| | |
|---|---|
| Sequence 541 | cMhvSH073b05 |

ACCGCGGTGGCCGGCCGANGTACCATCTTNCGAGATACTNATTCACGTCAAAATNCTCCTGCACCG
GAGGATNGGGGCACTTCCCAAGATGAAATGCTTGTCCCTCTGCCGCACCGAAGAGGCCAGCCAGT
GCGGAAAGCAGCAGCAGCAGCATCACCATCTTGGGGCTGGGTGGCTGGAGAAGGAACCTGGAGCT
TTTCTTTCAAGATGAAGGCANGTTNTCCAGATGCANAATCAGCCCGATTTGAGATGCCTGTCTTGG
TGACCTGGCCTCTCCCAAGCTCCCCGCGATACCTGCCCGGGCCGGNCGCTCTTAGGAACTAGTTGG
GAATCCCCCCGGGGCCTGCAAGGGAAATTTCGGAATATACANAGGCCTTTATCNGATACCCGTTCG
ACCCTNNGAGGGGGGGGGGCCCCCGGGTTACNCCAAGCTTTTTTGNGTCCCCCTTTTTAAGTGGAG
GGGTTTAAATTNGCGGCCGCCTTTGGGCNGTAAAATCAATGGGTNCAATAAGCCTGGTTTTCCCTG
GTGGATGAAAANTTTGGTTAATCCCCGCTTCCACNAAATTTTCCCACCACCAAAACCATAACCGGA
AGCCC

| | |
|---|---|
| Sequence 542 | cMhvSH073f04 |

CCGGGCAGGTACACAAGAGTTTGTCAGACAAATAAAATAAGAATACTTCACACACGTATCAACAC
CATACAAGGCATTATTCTTCACACAGTAACATCTAATGTGTTCTTTTATTTTTGAAACAGCAGGAA
AAGAGCCCTTTCCCTTCAGAGGAAAATAAAAACTTTATCTGTTGCTTAAGCCAAACTCCAGGGAGG
AAGGTGTGGNCCTCTGGGGAAAGTANAGGGATGGGGATGTATGGAGGANAATGGACCGCCCCTTC
ATAAAGCACTTCAGGGAGGAAGGAATTGCAAGGAGGGTTTCCTNGGGGACAGNTCACACAGANTT
NCCCGATCCTTGCCCCTTTCCTTCACTGCCACCACTTGCTTCACCGGCCGTCACACCATGGGGGGAC
TTGGCCCATTACTTTTCTTTCTTCAATAAGAATGGGGGAAACACNGTTTTTACNTCTTGGAGCCTTT
TTTTTCAANGACTTTTCTTTGGCAANNCGNCTNTCGAAATNCAGCTTCCTTTCTTCGNTGGGGGAAG
GGCCAATTNNAATTCCCACCGGGNCANGGANNTTAGNAATTGTCCCAAGGGGGNCTTCGGGGGGG
CNTTGGGGAAGCCTNCGGCCGTTCCCTTCCTTGGGGNCAACGGGGGTTAACCCTTTCGGGCCCCGG
TTTCTTAAGAAAANTNAGTGGGGAAATCCCCCCCCCNGGGCCTTGGCCAANGGGAAATTTCNGAN
TNNTNAAAGNCCTTNATTCGGAATAANCCCGNTNCCNNACCCTTNNAANGGGGGGGGGGGCCC

| | |
|---|---|
| Sequence 543 | cMhvSH074c08 |

AATTGGACTCCACCGCGGTGGCGGCCGAGGTACAGAACCCGACCAAAGTAGGCTGGTGAGGAAGT
CCAGGCTCCAGGGGAACAGACGCTGCCCAGTGTTCATAGCTTCCTGCAACTTGACAGAGCCTGAGT
TTGCCTCTTAGTGGGAGAATGAGAGAGAGCTGTAGTGTCACCTGACATTCCCCAAACCTTGTGAAG
CACGTTGGCCTAAGTGTGCCGTGATCCCAGCCCACACTAGCCTGGGTGCATCTGCTAATGGGAGAC
CAAATCTTTGTCCCGGGAAGCAAGAAGTGGGTGGGGAGTAATCGAGNCGGCCCGCCCGGGCAGGT
ACNGCGGGGATGATTCTGAGGGAGCCGGTGAAGCCACCCACCAGGGAGGCATGAAAAATGNAAA
AGGGACAGNGGCCTGACCAGACAGTCCTTGACAAGAGGNACGAAGAAAAAAAAGAAACTCGAAA
AACTTGGCCTGCAATGGGATTTGGGAACTACAGGAAGGATAAGCTTGAGAAAATTCAGCCCAAAA
GGGGGCTTGACTGTCATTTGGNAGCCGGTGGGCACTTGTTAAANGAAGCCAGCCCATCACCATTG
ATCCTGTTTTTTCACCACTTCACTTGGAAAGGACACCATTTTTATATACCCCAAGGGGGCGGGAAA
AGTTAAAAACTTTACTATTTTCATTTAAAATGTTTTGACACCAATTTGGGAAATTGGTCTTTTTAA

| | |
|---|---|
| Sequence 544 | cMhvSH090b03 |

AGGTACCCNGGGACCAGTANNTTGGNANACANTGCCTTCTGTNTTCTCGNGNGNGCNCTTGCTCCA
NNTNCTGTTCANGGCCAGCCNTGGCACCCTGCTCCTGGTTCTNTGCCTGCANTTGGGGGCCAACAA
AATGCTCAGGACAACACTNGGAAGATCATAATAAAGAATTTTGACATTCCCAAGTCANTACCTGN
CAG

| | |
|---|---|
| Sequence 545 | cMhvSH090b03 |

CTCCCACACTTTTGTATCCCTTTAACATAGGGACTAAATGCTCCCNTTGGTCGTAAANCATGGGGT
CATATTCTTGTAATCATGTGGGCTTTTCTTTTACTTAAATTTTGATCCTTGATTTCTCCTTGCCTCTTC
TTGTAGTCCAATGCTGATCTCTGTTATGGGGCGTT

| | |
|---|---|
| Sequence 546 | cMhvSH101a06 |

CCGGGCAGGTACGCGGGAGGGTGGCCCAACTGGACCAGCTCCTNNACTACAGGAAGAAGTCAGCT
GANTTTCCAGACTTCTATGATTCTGAGGAGCCGGTGAGCACCCACCANGAGGCAAGAAAATGAAA
AGGACAGGGCTGACCATACAGTCCTGACAGAGGACGAGAAAAAAGAACTCGAAAACTTGGCTGC
AATGGATTTGGAACTACAGAAGATAGCTGAGAAATTCAGCCAAAGGGGCTGACTGTTCATTGGAG
CGGTGGGCCACTGTTTAANAAGCAGCCATCACATNATCTGTTTTTCCACCACTTCACTNNAAAAAG
ACACCCATTTATATACCCCAAGGGGCCAGGAAAGTAANAACTTACTATTTCATTAAAATGTTTGGA
CCACCAATTTGGGAATTGTCTTTTAATTTTCTTGTCCAAGAAATGGCTTATTTGGAAAAATGTGAAA
TTGCCATTGGACTTTNGTAGCCATNATTTTCTTTTTTCTGCCAAAAATTATGACCATTNATTTANAC
CNTTGGCCTTTATTGACCAAATTNAACCNTGGTGCCTTAACTTGGCCTTTTTNGGGGAAAAAAAAT
GTTTTTGGTTCCTTTAAAATTTNGGGAAAA

| | |
|---|---|
| Sequence 547 | cMhvSH110a11 |

AGGTACAAACCCAGTTTGTTTTCAAAAAATCACAGNAGCAATGCAACTCATCACTCTAGAAAAGC
AAGCTTAGGCTACCTGAAAGATTTTCCCTTGGAAGTTTAGCGTATGTTTGACTAACAAGAATTCCC
TACATCAGAGACTCTAGGTGCTATATAATCCAAAAACTTTTCAGCCTGTTGCTCATTCTGTCCCATG
CTGGCAATAATACCTTGTCAGCCCATTACCCTTATTTTTGAATTGCTCCATCTCCTGGTGGGACTTG
TATCTTGTCTGCCATATCAAGAACACAAACCCCCTGAAGAGGNTCTGGATTTGGATTTTTTTTNTCT
TCATGCCTACCCTTTTTTTGGAAGTTTTCCAAGCCGCAATTTGGAAATGGAAATGGACAAGGGTGT
ATTATTTTGGATCCAAATTTTTCATTCCCCACCATTGCATTACCAACCTTCTAACTTTAAAATGGGG
TAACCCCTTAAANGGCCATTATTCAAAANGAAAGCCAGNACTGCATTGAATAAAACCGGNAANAT
TAAGAAAAAAAAAAGGAACCCTACCATTTTTATTTTTTGGGCTTNTAGCCAATTNCCTTTAACTCCT
TAAACCTTTTTTNNTNGGAAGAATTNGGAGAAGGNGGGGACCTTTNACCAANTTTNCCNCTTTTNT
TTAANCATTTTTNCNTNTATNNNCCTTANTNTTTTTT

| | |
|---|---|
| Sequence 548 | cMhvSH110a11 |

AGGTACAAACCCAGTTTGTTTTCAAAAAATCACAGNAGCAATGCAACTCATCACTCTAGAAAAGC
AAGCTTAGGCTACCTGAAAGATTTTCCCTTGGAAGTTTAGCGTATGTTTGACTAACAAGAATTCCC
TACATCAGAGACTCTAGGTGCTATATAATCCAAAAACTTTTCAGCCTGTTGCTCATTCTGTCCCATG
CTGGCAATAATACCTTGTCAGCCCATTACCCTTATTTTTGAATTGCTCCATCTCCTGGTGGGACTTG
TATCTTGTCTGCCATATCAAGAACACAAACCCCCTGAAGAGGNTCTGGATTTGGATTTTTTTTNTCT
TCATGCCTACCCTTTTTTTGGAAGTTTTCCAAGCCGCAATTTGGAAATGGAAATGGACAAGGGTGT
ATTATTTTGGATCCAAATTTTTCATTCCCCACCATTGCATTACCAACCTTCTAACTTTAAAATGGGG
TAACCCCTTAAANGGCCATTATTCAAAANGAAAGCCAGNACTGCATTGAATAAAACCGGNAANAT
TAAGAAAAAAAAAAGGAACCCTACCATTTTTATTTTTTGGGCTTNTAGCCAATTNCCTTTAACTCCT
TAAACCTTTTTTNNTNGGAAGAATTNGGAGAAGGNGGGGACCTTTNACCAANTTTNCCNCTTTTNT
TTAANCATTTTTNCNTNTATNNNCCTTANTNTTTTTT

| | |
|---|---|
| Sequence 549 | cMhvSH119h04 |

AGTTTCAGAACGACGGANAGCTCCCGCGTGAGGCTGCTGCCCCTCCTGGGCGCCGNCCTGCTGCTG
ATGCTACCTCTGTCGGGTACTTGNTTTTTTTTTTTTTTTTTTTTTTTTAAATTTGTTCACTGACCAACT
GGTTGTTCAGGAGCNCGTTGTTTAATTTCTGGATATTTATGAATTTTCTGAAATTCCNCCTGATTGA
TTTCTAGCTTCAAACTGAAAATATATTTGATATAATTTCTATCTTTCTTAATTTTACTGAGGCTTGTT
TTGTTTTCTAACATATGATCTATCCTGGAGAATATTCCATATGCAATTGAGAAAAATGNGCNTTCT
GTTGTTGGATTGAATATTCTGGATATATCTACNAGTCTTTTTAGAGTTANATTACTACCTTTCTCTG
TTCTCATCTTAACATCATCATGATGGACATTTTTATTTCATGATCAATGGATTTTCTCTCATCAAAT
AAA

| | |
|---|---|
| Sequence 550 | cMhvSH001c09 |

NCCGGNCAGGTACTCACTATGTGAAGTCTACCAAGCTCGTGCTCANGGGAACCAAGACGAATAGT
TAGAANAAAAAGAGCATNAAAAATAAAAAAAANANAAAAAGTACTCTGCGTTGNTACCACTGNT
TCCCGGGACTCTGCNNTGTTACCACTGNTTNCCGGNACTNTGCNTNGNNACCACTGGTTCCCGGGA
CTCTGAGTTGATACCA

| | |
|---|---|
| Sequence 551 | cMhvSH001g03 |

CCGGNCAGGTACTNNTTTTTTTTTTTTTTNTTTTNGACTATTTATTCACTATGGCAATTCCAGTGCCT
TGAGTGATGCCTGGCTTATCATGGGAGCTCANCACATAACAAATGCATACATGAATACGGATTCTC
CCTCTCACCCCAATCCCTTGGGATATGCTCTANTATCCACTGACTCCTACTCTCCTGGCTGCCTGNA
AAGGTAGGCATGCCCACCGATGTCGCTGANCAGCATGACCTTGGTGTTGGCAGGGANGTNCTGCT
TGAAGACTGGACGCTGCTCCTCTCCNATTAGTGTNTNGGGGTGCCCAAAAACATCCAACACNTTGG
CAGGTGNCGGNTCAAACAAATGAAACCAACCTTTAGCANTAACTGNCACAAACAGGTTCTTTCCTT
TATTACACACGTNCCCAACNCCAACGCAAGTCAGCATTCCCTGGCAGGAACAGGGTGAACCAAGG
GCCCGACTGTCATCATTTTTTATACACAGACACCTTTCCCGCTGGTGTNTNCCACCACCAGGTTCNT
TTAACGTATCGNTATTTAACNGTTTCCTAGGCAAAATTGCTTNCCGGGAAAGAAAGCTTTNCTGNT
TGAAATTTCANNNGGCCACGCCGCTTGAACGTAAGCTNAAATTGAACNTTATGGGGCACCTTCCA
ANNAAACCAAANGGNNGCCGGNAAGGCCCCCAAAAAAAAANTTTNCCTTGAAACCTTTCNGGNG
GGGAANCCCCCCGNANANCTTGGGCCCGTTTTNAAAAAAANTGGGGAATCCCCCNGGNGNNNGG
GGGAAATTCCNANANAAAANGGTTTTNTAAANACCCNGGNAACCCTTTTANGGGGGGGGCCC

| | |
|---|---|
| Sequence 552 | cMhvSH002e04 |

AGGTACTGGAGGCATGTGCCAACACACCTGTCTAATTTTTGNGNTTTTTGTAGAGACAGGGAAATC
ACTAACAGTTACTCTNNATAACTACTTGTTAAGTTAACCTACNAATNAAAAATGGCATGAAGCTTT
TACTGNCGGGGGGAAGTTTTCANATGTTACTACAACNTTAAGCCCAATACCTTGNGAGAGAAACC
AACATANATTGCACACANANCTTATTTGCAAAGTGCATATGGTCTAAGAGGCGATAGGATATGCA
AAATAACCATAATGTAGGATAGAAAATAAGGATGTATTAAGGAGCACACATGAAATCCTATTANA
GTTAAGAGAAGGTAGATAGAGCTCACTTGTTTTCAGATGTGGTGGTTCCTAAATCTTGAGACAGGA
GAAAAATAGATNGGCTTAGGGAT

| | |
|---|---|
| Sequence 553 | cMhvSH004g06 |

AGGTACTTTTTTTTTTTTTTTTTTTTTTTTTTTTTTTTTTTTTTTTTTTTTTTTTTTTTTTTTTTTNGNNTT
TTTTTTTTTTTTTTTTTTTTTTTTTTTTTTTTTTTTNTTTTTTTTTTTTTNNNTGGAAANCANATTTTTTT
TTAAAANNAAAACCNTNAANCNNTCCCNTTNTACCANAAAAANNNGGGNGGCTTTAAAAAAAAN
GGNAANCCNCAAAAAANNTTTTNNATATNCCNNANNAAAAATTTTNNAAANTTTCCNACAAAAAT
TTCCNAATAANNNGNNTTTTTTTAAAAANNAAANTTTTAGNGGGGNNNTTTTCCNCNCAAAANGT
NGTGTTAAAAAATTTTTNANNGGGNCNAAAAATTNGGNAAAANTNAATATTNTAAANNGGTGTTT
ANAAAAAAAAAAAAAANTTANAAAAANCNAAAAAAAAAAAAAGAAGGGNGAAAAANATAAAAA
TTTTNACC

| | |
|---|---|
| Sequence 554 | cMhvSH008f02 |

AGGTACTNGGGGTTGNTTAGCAGAGGCCGGAAGCGGTGGTTTTTAGCGGCTCTCTGGGTAGCAGG
GTGGTGTGATAGCGGCAGCGAGGGGCTCGGAGAGGTGCTCGGATTCTCGTAACTGTGCCGGGACT
TAACCACCACCATGTCGAGCAAAAGAACAAAGACCAAGACCAAGAAGCGCCCTCAGCGTGCAAC
ATCCAATGTGTTTGCTATGTTTGACCAGTCACAGATTCAGGAGTTCAAAGAGGCCTTCAACATGAT
TGATCAGAACAGAGATGGTTTCATCGACAAGGAAGATTTGCATGATATGCTTGCTTCATTGGGGAA
GAATCCAACTGATGAGTATCTAGATGCCATGATGAATGAGGCTCCAGGCCCCATCAATTTCACCAT
GTTCCTCACCATGTTTGGTGAGAAGTTAAATGGCACAGATCCTGAAGATGTCATCAGAAAATGCCT
TTGCTTGCTTTGATGAAAAAACAACTGGCCCCATACANGAAGATTACTTGAGAAAAGCTGCTGAC
ACCATGGGGGGATCCGGTTACANAATNANGAAGTGGGATGAACTGTACCCTTGCCCCGGGCCGGN
CGTTTTANAAACCTAGNGGGATCCCCCGGGCCTGCCAGGGAAATCNAANATTAAAACCTTATTTG
GATNACCGNTNNACCTTTAAAGGGGG

| | |
|---|---|
| Sequence 555 | cMhvSH016d01 |

ANCTCCGCNGGCGGNGCNCCCGCGGCAGGGACACACGAGCATCAAGGNAACAGGNCTGAGGANN
NNAAACGACTNTGTNATNAGANNNNAGAANNAATATTGCTCACACCTGCTACACCTTCTTGGGAG
CCAAGGGAAGCCTTTTCTGCAATCNCCCCATTTTGATNNAANCTNATCANCNATGGCTTGGGCNAN
CAAAATATTTAAAGGTCTNTTTCCCANCTCTTNCACTTATCTACTACATAAGGCTATAGCAATTAA
AAAGTCTTTCCTTTCCTGCCGCCGTACCATGGGTCCNNCTTGGGTAGCAACTTAGTGG

| | |
|---|---|
| Sequence 556 | cMhvSH021c01 |

AGGTACGCGGGGTGGCGAAACGCTGTCTCTACTAAAACTACAAAAATTAGCTGGGCGTGGTGGCG
CGTGCCTGTAATCCCAGCTACTCGGCAGGCTGAGGCAGGAGAATCGCTTGAACTGGGGAGGTGGA
GGTTGCAGTGAGCCGAGATCACACAACTGCATTCCAGCCTGGGTGACAGAGGGAGACTCCGTCTC
TAAAAAACAACCCCCCCCCCCCAAAAAAAAAAATGCATANCAAGCTGTAATGCTCTTTGTGTTTTA
GAATANTAGAGGTCTGGAAAGTTGTTTGCTTTTCCCCAGTTTTTTTTTGCTGTGTTACCTCTGAAGG
GAATTGAGGTAGAGGGGAGAGTTAGAAGGAATATTCGGCTTTTCTATTTTATATCCTCCTAGGTGA
AATTTTTACAACAAACATGTACCTGCCCGGGCGGCCGAGGTACTTTNTTTTTTTTTCTTATTTGCNN
NCCACTTTTTGNATTTGGNAAT

| | |
|---|---|
| Sequence 557 | cMhvSH027e11 |

CGCGGTGGCGGCCGCCCGGGCAGGTACGCGGGGATAGCCGTTTGAGGGAAGAAGGAGGAAAATT
ACCCGGTATCGTTAGAGCTACACCAAAATTGCATTGAGCCAAACTTGCCACCAAGAGCCCAACAA
TCACCATGATGCTGAGCACGGAAGGCAGGGAGGGGTTCGTGGTGAAGGTCAGGGGCCTACCCTGG
TCCTGCTCAGCCGATGAAGTGATGCGCTTCTTCTCTGATTGCAAGATCCAAAATGGCACATCAGGT
ATTCGTTTCATCTACACCAGAGAAGGCAGACCAAGTGGTGAAGCATTTGTTGAACTTGAATCTGAA
GAGGAAGTGAAATTGGCTTTGAAGAAGGACAGAGAAACCATGGGACACAGATACCGTTGAAGTA
TTCAAGTCTAACAGTGTTGAAATGGATTGGGTGTTGAAGCATACAGGTCCGAATAGCCCTGATACT
GCCAACGATGGCTTCGTCCGGCTTAGAGGACTCCCATTTGGCTGTAGCAAAGGAAGAGATTTGTTC
AGTTCTTTTCAGGGTTGGAAATTGTGCCAAATNGGGATGACACTTGCCAGTGGNACTTTTAAGGGG
CCNAAGCACCANGGGAAAGCCTTTTGTTGCAGTTTTTGCTTCACAAGGGAGAATANCCTTANNAA
NGCCTTTAAAGNAAACCCCAANGGGAAAAGAAANTATGGGGCCCCAAGGTTACCCTTTGTCCGCT
TCTTANAAACCTAGGNGGGATTCCCCCC

| | |
|---|---|
| Sequence 558 | cMhvSH038a05 |

AGGNACCTCTCGGAGGGGCCCTCCTCCTGCTCCATGGGGATCCGCAGCGCCAGCCGGCCAGGGTTT
GAATTAGTCATTGTTNGGAGGATACAAATAGATGAAGATGGGAAGGTTTTTCCAAAGCTGGATCTT
CTCACCAAAGTCCCACAGCGAGCCCTGGAGCTGGACAAGAACAGAGCCATAGAAACTGCTCCTCT
CAGCTTCCGAACCCTGGTAGGACTGCTTGGAAATCTGAANCTGCTCTGGAAAGCCCTNNATAAAAT
CCGCTTTGTTGCAAGAGGGAGGAACAACTAGTTCCAAAAACAGTTGGAACGTTGGTAGGCATGAA
AGCATGCTTGCCGNTGGGAGGGAACATGTCAAATNTTTATTCAATTATTAAAACATTTTGCTATTTT
TCTGCTTAGNAAACCACACNCCTTGGAAGACCGTGCCTGTCTATGGCAGATTTATGGGCACCATTA
TTATGGGAAACTCTTCATGACATGGAAAAAATTAAATACCAACTAGTTTAAGTTATAAAAATGCCA
NNNTGNCTTTACTNATACCACCTGGNGCTNAAATTATGGATCCCTTTTACCAACNTCCCCCGCCCC
TTTAAANNTTTTTTTAAAAANAACAAANGGTTCCCCNNTGNCCGGGGNCNTGGGGCCNTTTTTTNA
AAAAA

| | |
|---|---|
| Sequence 559 | cMhvSH039f09 |

CCGCGGTGGCGGCCGCCCGGGCAGGTACCTGTTTTGTTTCCTGATTATTCCAGGATTCTCTCACTAG
ACCCTAAGCCTCTCATTCTGCTGTAGGTCAGATTCTCTATTCCTTCTCCCTAGCCCAGAGCCTTGCC
AGCACTTGCGAAAGTTACGGTTAGAATGTTCCCTTGCCTAGTCACCTCTTTGAAAAAAACACTGTG
ATGTTACATGACTGCGATTCAAATCAGACACTGTCTGCTTCCCACATGTATCTCAGACAGGTTTTAT
TTAATGTTTCTTGTCAGAATATTGTAAATTCAAAAGGATGACTTTAAATAAATGTAAACAAAGACA
AACTTGTGGTCTTTTTGTCTGGAATTACTTTCACAAGAGATGGAGCTTGCAGGGGAATTTACTGNC
TGACCAGTTACTAATGGTGAGCCCTTGC

| | |
|---|---|
| Sequence 560 | cMhvSH043g09 |

ACGCGGATCTTTCCCAACTTTAAATACTCTTTTAGTTTCTATAGGGAAGGAAGAGTTATTACAGGTT
TTTTTTTTAATTATTCTTTAACTTTAGATACTGCCAATCTGATTTAAAATTCTCCAAGCTTAATTCTG
TGCAACAAACAGAACCACACAAGCAGCCAGGCACTGTGGCTCACTCCTATAATCCCAGCATTTTTG
AGGCTAGATGGGAAGATCACTTGATCTCAGGATTTTGAGAACCATCCGGACAACATAGGGAGACC
TCATCGCTATTTTAAATAATTTTAAAAAGAAAAGAAAAAAAAAGGCCAAAGTGCTGGGATTATAG
GCGTGAGCTACCGCGCTCGGCCATTATATCTAGATTTTGAAACCTCATGTTTGTTTACCAAGTAGTA
ACAGGTGTACCAGCAGCTTCCAGGAATA

| | |
|---|---|
| Sequence 561 | cMhvSH044e05 |

CCGGGCNNGTACGCGGGAAGTGCGGGGCAGGACAAAGGGCTCTTTGCACAGCAGGGAGGCAATG
TTGGTGGGGGAGGGGCAGGAGGTAGGAAAGGCAAGAGGAGGAGGTTCTTTTCCCTGGGAGATTAT
TCANNTTTGGCATACANTTAAAGAAATCATTTTTAGTTCCCACTCAAGCATTGAATTTTTGCCAACC
ACATACTATTAACCCCAAATTTGATACATTTCAGAATATCTTGTAGGGATCCATTCTCGCCNTAAA
AAAAATAATAANAAAAAAAGGTCCCTCGGCTCGNTCTAGAACTAGTGGATCCCCCCCGGGNTGTA
GGAAATTNNTATATCTAAGCTTNTTCGNATAACCCGCTCGNACCTTTNAGGGGGGGGCCCCGGGTT
CCCCAAANTTTTTTGGTT

| | |
|---|---|
| Sequence 562 | cMhvSH045b02 |

CCGGGCAGGTACTTTTTTTTTTTTTTTTTTTTTTTTTTTTTTTACGTTAAAAAAAGTTTTATTTAGGGA
GCTCCAGGGAATGCGGNGGGAAAGGANAGGTGCAGTGTCATTGCCGCCCTCTCCTCCCACCTAGN
GCATTAATAGNGGATGGGAGCATNTGACAGAAGTGAGATCAGGCAGNGGGTGTNTGCNCCCCACA
GCGCATGTTGGCTGGAACAGCAAAGNCTATCTGCTGAGGTTTAGGCAAGTTCAGGNTGCCCATGA
TTTTGACAAACTCCTCACANCTGAGGGTGAGCCNAGGGTTCAAAGTCCTTTTCTTCTCCACGGGGG
ACACTGTGAACCCATGGTAATCGNGAGCNGGGTANATCANACNGCCTCCTGGAAGNGTGAANATC
TTTTNATGGCCCCNAGTGGTGCAAGGTCTTNGCACAACCTTGCTTGGAAGAACTTCCGCCCACCCC
CACNGATCAAACAGGGGCATCTTCCAATNAAAGCCCATTCTTNTGGGNCATTTTCANGGANNAAA
AGGGGACACCAANCCTTGGGGNTGGTGGCCCAANGGGGGTNGGCNCCTTGGTTCNTNCCAACNNC
GGAAAAACGCCCCCNAAANCGGGATTGGGAGNTCTCCCNTCCCCCAAATGGGNTAAAAGTTCAAC
CCTGGGGGCCCCCCCTAAAAGGCCGGGAANAAACCCCCCCNTCCCCTTGGGCCGNTTTTTGAANA
AAANTNGGGTNNCCCCCCGGNCTTNTAANNAAANTTNNANTTTTCACNCTTTTNAANNNCCCCCN
NCCCCNNANNGGGGGGNNCCCCNNNCCCCCCCCCTCTTTTTTTNCCCTTTTGGGGGGGG

| | |
|---|---|
| Sequence 563 | cMhvSH047h11 |

CGANCGGGCAGGTACTTTTTTTTTTGTTGNTTTTTTTTTTTTGGCTTATCACACCTGATTTTCTACAG
TNAGCATAAGTTGCACATGGATAATAACACACNTTNTTAAAAGGCNNAAACAACAACTATGATCA
CAATTTAAAGGCAGAAAAGTGCTATTATCTTAACAGAACATGGAACATCCATGTTCTATGATAATA
ATAAAGTTAGGCAAAGTTAATATCAAATAACCTGATATTCAATAGCCTAGTTTTTAATTAGTTTTA
GTAACACATATGGAAGAATCTGTTATGAATAAAAAACCATGTNGGCCGGGCACGGTGGCTCACGC
CTGTAATCCCAGCACTTTGAAAGGCCAAGGCAGGCAGACCACGAGGTCAGGAGTTCGAGACCAGC
CTGGCCAACATAAGTGAAACCCCGTNTTNTACTAAAAATACAAAAATTAGCCCGGCATGGNGGCT
TGTGCCTGTGATGCCAGCTACTTGGGGGGCTGANGGAGGAAAATCACTTGAACTTTGGAGGCGGA
AGGTTGCAATGAGACNAGAATNGGGGCCCTGCCCTTCCAAACCNTGGGNGACAGGAACCAAGGA
CTTNCATTTTCCGGGGGAAAAAAAAAAA

| | |
|---|---|
| Sequence 564 | cMhvSH056g11 |

AGGTACTTTTTTTTTTTTTTTTTTTTTTTTTCCCTATTTCTCANGNTTTNATTTTCANACTTTGCTAAT
TACTTTCTTNTAAANGNCTTCATTTTCAATGAANNTTTTNTAGCCATTNTCANTNTTTNTGTTTTAN
CANACCCNTTTANATTNTTCNCATTTAGCATAGCAAATGTTATATTTAATTTTATTTCTTGACCCNC
NTAAGGTTCNTAATNAACCGNATGGGNTTTTGGTTACCCCNTTTTTANAANNGTATTANCCNATTT
GNNANANTTNTTACCCANCCCCCNNTTGNTAATNTGGAGACTTANGACNNTCCAAAAAAAGGTAT
ACCCTCATTNTGAGGGCNCNNCAAAAACCCANNTTTTTNCNTTTATTTGNAAANNAAAAAGGTAA
CCANTTTTCCCCAATTCAAGGAAAGACTTGGGGGGNNAANATTTTCCCGGCCC

| | |
|---|---|
| Sequence 565 | cMhvSH057d12 |

AGGTACTTTTTTTTTTTTTTTTTTTTTTTTTTTATGAGATGGAATCTTGCTCTGTCACCCAGGCTGGA
GCATAGTGGCATGATCTCAGCTCACTGCAACCTCCACCTTCCGGGTTCAAGCNACTNTTGCGCCTN
AGCCACCCAAGTAACTGGGACTACAGGCATGCACCTCCACGCCCTGCTAATTTTTATATTTTTAGT
AGGGATGGCTTTCACCATGCTGGCCTTAANTGATCCGTCCGCCTTGGCCTCCAAAGTGCTGGGATT
TCAGGCAAGCGTTACCACACCCGACCCCTCACTAGTATTTCAGCATTAATGTTCCCTCTTTAACCAG
NGCTTATTATGAGTATACACAAACAACATTGCCTGACATAANAACAAGTTGAACCCACAGTGGAA
TCCCTACAGNGGCAGACAGTGGCAGCTGANAGTGACAGACCAACGGGGGGAAAAGCCACAAGCC
ATCTCCTGTAAGCTTCACTGCCATNACCTGAGCTCATGGCACACACCTGCTTTACCTNTAAGCGAG
GNGCTGCTCTTTACATTACCACTCTGGGAANAANCAGGCCCAACCAAACCCCACCANGNCGNTTT
AGCTTTTCAAGGGACCCCAAGACACATGTGTATAAAAAGCCANTTGCATGTGGTGNGNGGGGGGN
ATGAAATATANTGCCAAATATTTACCATGGNGGGANAGGNGGGGGGGAAANTNAGGNANTNTAA
AAAAAGCTTTTGGNGGGAAAAAGAAAAA

| | |
|---|---|
| Sequence 566 | cMhvSH058f01 |

CCGGGCAGGTACTTTATATGACTTGAATATGTTAAAACATATCAAAACTTGTTTCATGGCCCAGAA
TATGGTCTGTATTGGTAATATGTTTCATGTGCACTTGAGAAGAATAAATTTTGCTGTTGTTGAGTAG
TCTTCTATAAATGTCAACCAAGTTAAGTTGGTTGATAGTGTTTTTCATGTCTACTATATCCAGGCTG
ACTTTATGCCTACTTGTTCTATCAGTTATTAAGAGAGGACTATCGAAGTCCCCAATGATAATTGTG
GATTTGTCTGTTATTTTTTGTAAGTTGTATCAGTTTTTATTTAATTGATTTTGGAACCTTTTNNNNCT
AGGGNCATAGAACNTTTAAGGATGGCCANNGTCCCCTAANTTACNTGAACCCCCTTTTCATTNTTG
AAATGAACTTCCNTGGGATCTTTGGTCTGNAAAGCCNTTTTGGGCCAANNTAAAANAAGACGCCC
GCAGCANCTTTTTGGGGGNNCTAGGNTNAAACTANGGTATATCNTTTTTNCNATCCCCTTTAACCT
TTTTAAGGAATTTTGG

| | |
|---|---|
| Sequence 567 | cMhvSH062a08 |

AGCTCCACCGTGGTGGCGGCCGCCACTCTGGTTTTGCATCNTCAGGANACNGCTCGGGGCCNGNG
NGCTTCTCCTANNNNAATNNTTTTNTATAAGTGGCTCACGCCTTCCATAGCCACATCATCTCGGTTC
GAAATAGAACCCCATANAGAGGTAGGTTGTAGGAGGCCTGCAGGTACCTA

| | |
|---|---|
| Sequence 568 | cMhvSH062a08 |

NANGGAATTTCNATATCAANGCTTATCGATTACNCGNCGTACCTTAGAGGNGGGNGGCCCNGG

| | |
|---|---|
| Sequence 569 | cMhvSH062c12 |

AGCTCCACCGCGGTGGCGGNCGAGGTACGCGGTNGCCTGCGCCCTCTCCTATAAAGCNGACGCCG
AGCCGCGCTGCGACGCTGTAGTGGCTTCGTCTNCGGTTTTTCNNTTCCTTCGCTAACGCCTCCNGGC
TNNCGNCAGNCTCCCGC

| | |
|---|---|
| Sequence 570 | cMhvSH062c12 |

ATGCACGAATTCTGATATCAAGCTTTATCGATNCCANTTTACCTTNCAGGGGGGG

| | |
|---|---|
| Sequence 571 | cMhvSH063h03 |

AGGTACTGTTTAATCTTCTCCATGGGGCTAACAGAGTGAGTGTTAAGAGCAGTGTGGCCATCCTCC
AGCTCACTTGGCCGAACACTCAGCTCCGGGATGGTTCGAACGAATCTGGGGTGACTTATTGGGAG
ATACTTGAATGTCTTCATGTCTCGCCCGCCAATCACTCGGGCAGTGACCGTCTTCCCAACCTTCAGC
TTGGTAGTAGGAGAGGTGCCCTCTGGAACATCATTNTANAATGTGGGAGGCATGGATACAGCCAA
ATAANTGCCCATCTTCCAGAGTTCACAACCACATGGGGGTAGGCCTTTAAATTGGACCTTGGACCA
GTTCNCCTTGTNGAACCAANTGTCCCCCGAATGGGANGAGGGGTGNTGCTTNTTTATGGGTCCCCT
TACNAGNTCAAGANGCTTGGGAATCCACTTTTNTTTCNATCCCTTCATTCAAACCTGGTTCTTCANN
AAGNTTCCTTTCNTGGGGTTCCGGGCCCTTCAATGGGGACCCTTCTTTGGGCAANTNCCGGGNGCC
CCCCTTTCCACCAAGNCCCCAAAAAAGG

| | |
|---|---|
| Sequence 572 | cMhvSH064b08 |

TCCACCGCGGTGGCGGCCGCCCGGGCAGGTACTTTTTTTTTTTTTTTTTTTTTTTTTTGTTTTTTTTTT
TTTTTTTTTTTTTTTTTTTTTTTTTTTTTTTTTTTTTTTTTTTTTTTNATGGNNNCCACTTTNTTGCCNAA
NCNTGGAACTTGGGGNAANCCTNNACCTTCAANAACNNGCAAAAAAAANGNTGGGGGGNTTTTG
GGANNNNNCCNNNCCCAANGGGGAAACTGNCCGGGGAAATTCCNAAACNGGGAACAGGGGGGG
GTCCCCNTGACCCCCNAAAANNTTTTTCCCCCCNCCCTTNGGGGNGNGGNAGGGNACNNAAAAAA
AAAATGGCNTNCCAGGGGTTTTTCCCATNNTNCCTAANCCCNCNATNGGGGCCCCATTTNNAAANT
NCCCNGGGGNNGGGAAANGTTTTNGGAAAACGGCTNCCCAANAAANTNTCCNNNCCACCCNGGG
GTTTTTTNTTAAANCTTNNTCCCNAACCNNTTTGCCTTTTTTTACCCNTTNANAAAAANNGGCCNCC
ACANGNGGGGGNCCAAAAAAAAAATAACANAATTNCNGGGNNAAAANTTNTTNTNGGGGGGGNA
NATNTTTTTTNNTTTNGNCANTTNGGNAGAAAANGGGAAAAAAGGGGNGCTTTNCCCCCANCTTTT
NGNAAACCNCCTTTTTAAAGGGGGAAACNGNNCCCCCCTTTTTTTTTTTTTTTTTTTTTCCCNTTTAA
AAANACCANNCCCCCTTTTTTTTTTTNCCNATTTTTGCNCCCCAAATTTTTNCCCGGTTCCTTTGGN
NNNTTTATNNAAAAAAAAANNGGNNCCCCCCCNGNNNNCGNGGGANTTTTGNNTTTATCANNTTT
TTTNTTCNCCCCCCCCCCCCCG

| | |
|---|---|
| Sequence 573 | cMhvSH070a02 |

CCGCGGTGGCGGCCGAGGTACTTTTTTTTTTTTTTTTTTTTTTTTTAAGGAAAAGGAGACTGGAAGA
AGAAAAATAAGTATTTNTGGCAGAACTTCCGAAAGAACCAGAAAGGAATAATGAGACAGACTTCA
AAAGGAGAAGACGTTGGTTATNTTGCCAGTGAAATAACGATGAGCGATGAGGAGCGGATTCAGCT
AATGATGATGGTCAAAGAAAAGATGATCACAATTGAGGAAGCACTTGCTAGGCTCAAGGAATACG
AGGCCCANCACCGGCAGTCGGCTGCCCTGGACCCTGCTGACTGGCCAGATGGTTCTTACCCAACNT
TTGATGGCTCATCAAACTGCAATGNGAGNTTATCATGTCTTTGACATCTTGATCACCTACNCCGAT
AAGGGACAGTCTTCACCATTTTAGTCTTTGNATTTCTTTTCGAAACTTNCGACTCGCACCTGGGTNT
GCAAAAGAGGGNGTCTTGTTCATATANAATNGNNTATTTTCTCTACCCTGACAGAGACTNAATTTT
ACAGTCAAAAATANGGGTNATCATNCNNGGGGGTTTTGGTTTTTTT

| | |
|---|---|
| Sequence 574 | cMhvSH071f03 |

ACCGCNGTGGCGGCCGAGGTACAATCTACTTANTCAAGCATAATAGCACTAGGCNGAATAAAAAA
TTGCACAGACCGTATGCAGATTTTNCAAGATAGCATTCTTTAAATTCAGTATTCACCTTCCAAAGA
TNGGTTGCCCATAATANACTTAAACATATAATGATGGCTAAAAAAAATAANTATNCTGANAATGT
AAAAAAGGAAATGTAAGTCCACTCTCAATCTCATAAAANGTGAGAGTAAGGATGCTTAAAANCAA
AATAAATGNGAGGTTCTTTTTTTTTTCTATTTTCCCGNNTTATTCAATGNCAANTCTTGCCTNCTTTT
GATAATGNCCTTTAANGGGGTTTACCCCCATTTTTAAANTTTAAGGAAGGGTTTGGTAAATGGCCT
AATTGGGGTTGGGGGAAATTTGGAAAAAAATTTNGAATCCNAAANTTATTAACCACCCCTTTGGTC
CATTTTTNCATTTTTTCAAAAAATTTNGCCNGGCTTGGGNAAAAACCTTTCCCAAAA

| | |
|---|---|
| Sequence 575 | cMhvSH071g11 |

CCGGNCAGGTACATTCCATTANTTTTCANTGTCACCTAAGGGTCAAGGTTTAGGGGCCTGACACAN
TAGTGTCACTCAGGCTGTNGCCCCAGNTGTAAATATCAACAAGGAACTNTTTTNTCCTACCCAGNG
GTTTTGTGTNTNCTGCAGTATTCATAATNTATAAAAGAATGNTTAACTGTGAAGTGAAATCATATC
TACAAGTCCCNTACAACANTTTACTTNACAAANACNATTATTNTNCCANCCCTNAACTCAAAAAAG
CCACNCAAATACTTANAGTNTNNTTNCCAAANTNNCNCACAAGCTGGTCCTTGANGNACAAAAAG
GTCTTTCCCAAAGANGCCTTGGGCTCAGGGAAAANGCCCC

| | |
|---|---|
| Sequence 576 | cMhvSH073g05 |

AGGTACAATCTAGTTAAACAAGCAGAATAGCACTAGGCAGAATAAAAAATTGCACAGACGTATGC
AATTTTCCAAGATAGCATTCTTTAAATTCAGTATTCAGCTTCCAAAGATTGGTTGCCCATAATAGAC
TTAAACATATAATGATGGCTAAAAAAAATAAGTATACGAAAATGTAAAAAAAGGAAATGTAAGTC
CACTCTCAATCTCATAAAAGGTGAGAGTAAGGATGCTANAAGNCAAAATAAAATGTAGGGNTCTT
TTTTTCTANTTTCAGTTATATCATGCCAGNCTGCTTCTNTNTGATATTGCACTTAGGGGTTACCCAT
TTTAAANTTTAGGAGTGTTGTAAATGNCAAATGGTTGGGGNAATGGAAAAGATTTNGATTCAAAA
TTAATACCACCCTTGGTCAATATTTCAATTTTCCAAAATTGGCNGNGNCTNGGGTAAAACCTTTTN
NCAAAAAAAAAAAGGGGGTTNNGGGCCNTTNGNAAGGAAAAAAAAAAAAAAAAAATTCNAAAN
ATTTCAGTAAANNCTTNTTTTTTAGGGGGGTNTTGGGTGTNTTCTNGAATTANTTGGGCCNGGAAC
TAAAGGAAATANCCAAAGTTCCCCNNCCCAANGGNAGGAATTGGGNAAGCCCAATTTNTNAAAA
AATTAAAGGGGGTTAAANTGGGGGCCTTGACCAAGGGNNAATTTAATTTGGCCCAAGCCATTGGG
GGGACCAAGAAAATTGGANCCAACCAAGGGGCTTTGAAAAAAGG

| | |
|---|---|
| Sequence 577 | cMhvSH075b05 |

TTTTTTTTTTTTTTTTTTTTTTTTTTTTTTTTTTTTTTTTTTTTTTTTTTTTTTTTTTTTTTTTTTTNAAAA
AANNNTTTTTTTTTNNNNTGGNNNNNAGGGNNAANNCCCCCNCANTNTTTTNANNAAANCAANNA
AANANCTTTCNGGGGGGANANNNNTTTTNNNNNNNNNANNNCCTNGGGGGGCAAAAAAAAAANN
NNGNNCCNTTTTTTTNGGGGGGNCCCTNGGAAANNCCNNCCANGGGGNNTTTNAAAAAAANNGCC
CNTTTTTTTTANCNNTNTCCCCCGCNAAANAAAAAAANTCCCCNANNGNNCCCNGGGGNCCNNAA
AAAAGGGGGGGG

| | |
|---|---|
| Sequence 578 | cMhvSH092d02 |

AGGTACACAAGTAACCTGCTTTGTCTGCCCTAAGCGGTGGGCCCTGTCCATGGCCTGCTGGTCCAC
AGTGGGGTTCCAGTCGCTATCATAGAAAATCACTGTGTCTGCAGCAGTGAGATTGATACCCAGTCC
TCCAGCTCGTGTGCTTAACAGGAACACAAAGATGTCATTCCTGTTCTGAAAATCAAGCAACCATGT
CTCGCCTCTCCGAGATCTTGGATGAGCCATCAAGCCTCATGTAAGTATGCTTCCTGTAAACCATGT
ATTCCTCCAGTAGGTCTATCATCCTGGTCATCTGGGAGTAGATAAAGGACCCTATGCCCTTGAGAC
TTGAGCCGAGTNAGCAGGACATCAAGGGGCATACAAGCTTTNCCTGTCAGTGATGANGCTTCTCCT
TGCCTGGAATCCTGATGAAAAGAACCAGCCCATTCTTGANGTCTNAATGCTNCACAGAACCTTCCA
AGCTGGGCTTTTGGGGAAANAAACTTGGGGAATCGGTCNTATTTTAAGCCCAGTTCTNGCAAGCCC
AAGTTTCAANGGGGGCCCCCATTTTNAACAAAAAAACTGGNTTTGGGCTTGGCCAANAACTCCCTT
CCTTTCC

| | |
|---|---|
| Sequence 579 | cMhvSH093c11 |

CCGGGCAGGTACCATAGTTTTTAAACAGGAAAAAATACTTTACTTTTGACTAAAAACTGGCCAGAA
TTTCTCATACTTCTCATTTTAGGGCTTTAGATCTCTGCATCCCGAAGCACAAATTTAAATATAAAAA
TTAGATTAACTGTTCGTATGTCTATCAGAATCAAAGTTTTTTTCCTTTTTAAAGATTTGTGGGTTAC
CCTAATATAAGCTAGAATTTTAGTTTTATAATTTTTTTCTTTTTTAAAATTGAGATGGGGTCTTGCTA
TGTTGTCCAGGCTGGTCTCAAACTCCTGGGCTCAAGTGATNTGCCTGCCTCGGCCTCCCAAAGTGC
TGGGATTATAGGCGTGAGCCACCGCGCCCGGCCAAACTAGAANNTTAATATTTTTCACCTCCTCCC
AATCAGGTAGAACATCAATAGACTGGAAGAAGATACTGNTNAAGATGTTTCTTTTAACAAAAAAT
TTCACACGCCAAAAATTTAAGATTTTTNCCATTATTGAAGACATTATTNTCAAAAATCTTTCCTATA
ACACTTTTTAGGGGAAGAAGGTGGAAAAAAATACCTTAAAAAGGTCGCATCTTAACCGGGGGGGC
TCACTTGACCGATATANNTTCTTTAGAATAGAAAGGTCATTCACCCCCAAANGGTCTTTATTAATTT
TAAATTNAAGGTTAAAAACCCACNGGAGGACCCTTTATTAAACACCATTTTCNCCAACCTCNNAAN
GGCTAATTTTTNTTNCTTTCCNATATTCCAAAACATTCAAACCAAATTTTGATGANTCATNCCCAAT
NGGGCTNGTAAAAANNATTGACCCCAAAAACTTTTTTT

| | |
|---|---|
| Sequence 580 | cMhvSH094f06 |

CCGGGCAGGTACCTNTTTTTTTTTTTTTTTTATTTCAAAATAAANNTTANAAAAANNGGCNACCTNA
NTGNGNTTTNTTTTTTTTTTNNAAAAAACCCTTTTTGATTTTTNACCCCNCNCTTNGNGCAATGNTG
NNAATANNNNTTNNNNGAANCTTTNCCNCCCANNTTAAAAAAANTNNNNTNCCNAAACCCCCNAA
ANNNNNGGNANTNNNNGGNTNNNANNCCCCCCCCNGNNAANTTTTTNAATTTNAAAAAAAANGG
GGNTTNNNCNNTTTNGCCNNGGNNNNTTNAAAANNCNNANCTTTTAAANNNCCCCCNTTNGCCCC
CNAAAAGGGNGGNANNAAANGGNNNANGNCCCCCCCCCCN

| | |
|---|---|
| Sequence 581 | cMhvSH095d01 |

AGGTACTTTTTTTTTTTTTTTTTTTTTTTTTTTNAANNNAAAAAAAAAAANTTTTTTTTTTNNGNAAA
AAANAAAATNNNNNGGNCCTTTNNNANNNCCCCCCNTTTNNNTTTNGGNNTTTNNNAAAAAAANA
ACNTTTTNNAAAAATTNGNNNNAAAAAAAACCNNNTTTTTTNNTTTTNNGGGNCNGGGGTTTTNCC
CCCCCCCCCCCCCTTTTTTTNNNANCCCCCCCCCCNNGGGGGGGAAANTTTTTTCCAAAANNNGGG
GNCCAAAAAAAAAAAAAAANTTTTCCCAAAAACCCAAAATTTTTAAAAANCCCCGNCCNTTTTTN
NAAANGNCCNNTNNTTTTTNNGGNAAAANGNCCCNTTGGGGNNTCCCGGGGANCCCNCCCNNTTT
TTNAGGGNCCCNCCCNTTTTTTTNCGNANACCCNCNCCCTNGGGGGGCCCCAAANACCCNTNGGG
GGGGGAAAAANCCCNAANNGGATAAAAAANCCTTNNGGTCNGGGGGNAAAAANNNAAAATCTNC
CANGGGGCTTNANNANAAANTTTNNCCNCTTTCCTTTTCCCCCAGGGGGAAAAAGGGGAAATTTTT
TTTAANNNAAANAGGGCCCCNCNGGGGTTTTTTTTANNGGTTTNAAAAAAAAAAAATTTTTTTAAA
AAAAAAANATTCCCCCTTTTTTTTTCCNNGGGGGGGCNCTTTAAAAAAAAANGNGAACCCCCCCC
GNCCNGGGGGAAANTNNNTTTTAAANNTTTTTNNTTANCCCCCCCCCCCCCNC

| | |
|---|---|
| Sequence 582 | cMhvSH095d07 |

ANGTACCCGGNGGCGGAAACCACCCNTTCAAACGTCTGCCCTATCAACTTTTAANGGTATTCCCCG
TCCTACCATGGTGACCGCGGGTNACAGNGNAATNNAGGTTNAATTTCNNAGANGGANCNNGATAA
NCTGNTACCACATNTANNGAAGGCNTNACGCNCGCNANNTAAAAATGTNANCTAAAANANGAAA
TANGTTTGTNGCNGANNTANCTNTTNAAAATAAGGTCNNCCCNGAGTAGGGGTAANACCTCCAAC
ATGACTGGTATCCNTATAAAANGGANNGGGGGGGACACAAAAACACTNTNACANGNNTAATGCC
NNATNCTGATNACCGCAGAAATTGGGGTATTGTTTCTATTACCCCAGGGAATCCCAATTTTGCCAG
TGACCCCCAAAANTTTAAGGAGAAGCCTGGAACAAATTCTTCTGCACAAGTCCTNAAAANGAACC
AGCTTTGCTTAACCCCTTNATTNTAAACTGCCNGNCTTNCAAAACTGANAATAAAATTCCTGTTAT
GTTAAGCTTGCCCTTTTGTGGGGGCTTTTNTTTGGGCCNNCCTTTNNCCAAATTTATTNNAAAACCC
CGGCCNTTGAAAAAAAGGNCCAAAATTTTTTTTCCTAAAAAAGCCTTGGGGCTGGNGGNNGCATT
TCTTGCANTNCCCNTTTCTTTTGGCCCTGGGCNCTTTAATTTAAGGCCTTTNNCCTTTTTGANTTTAT
TTCCCCTTGGCCCCCAAAATAAACTTCAACCCTTGCNCCCCTTAAAAATNAAATGNTGANTTNTTT
NAAANCCGTGGNTTTTTTTCCCCCATTTTTTTTTTT

| | |
|---|---|
| Sequence 583 | cMhvSH099d01 |

ATGGAGTCTTGCTCTGTTGCNCAGGCTGGAGTGCAGNGGCGCGATCTCAGCTCACTGCAAGCTCCG
CCTCCCAGGTTCACGCCTCCCAGGTTCACGCCTCCCGAGTNGCTGGGACTACAGGCGCCCGCCACC
ATACCTGGCTAATTTTTTGTATTTTCAGTAGGGACGGGTTTCCGCCACGTTGGCCAGGATAGTCTCA
ATCTCCTGAACTCGNGATCCGCCCTCCTNCGCCTCCCAAAGNGCTGGGATTACAGGCGTGAGCCAC
CGCACCGGGCCTCTTGTCACTATTTAACAAAGCATAANGGCTCCTCTCTGCCTACTCTACCAGATC
CATGCTCTTTAGCCTGCCAGGCCAGGCTGTCCCTACCTCACATCCCCTGATCAGCTACATTATAATC
TAAGGCCTATCTCCTNTTTAACCCTGAACGTACCTCGGCCCGTCTAGAACTAAGNGGGATCCC

| | |
|---|---|
| Sequence 584 | cMhvSH099e09 |

ATGAAGTTTGTTTTGNCGANAAATTAGGTTACTTGNGTATCAAAGCTTATTTTTAAATNGNGTTAG
GGNGTANCCAANCCCTTTATTCTANANATNCTTTAGCTGNATTACTAANACATAGCTAGTATCTCT
ACTTAANGCTCTGGGTNGTAAACAGGGNCTTTCCATNGTTCTACCTTTAGGATTTCAATAGTNTAA
AACCGGTTGGTTTTTGAT

| | |
|---|---|
| Sequence 585 | cMhvSH102g10 |

TCCCCGCGGTGGTNGCCGCCCGGGCTNGTACGCGTTCATCTGTAATCTCAGCCTCCCGAGTAGCTG
GGACTACAGGCGCCTGCCACCACACCCGGCTAATTTTTTGTATTTTTAGTAGAGATGGGTTTTACC
ATGGTCTCGATCTCCTGACCTCCTGATCTGCCCACCCTGGCCTCCCAAAGTGCTGGGATTACAGGC
GTGAGCCACTGCGACCGGCCCACTTTTTCTTTTTACTTTTAAAAATGTGGGNTAATAGAAATTTATG
AGATTATATTTATGGTTCATACTACGTTTCTTTTGGACAGTGCCAGAGTGAATCAGATAAGCTTGC
ATTTTAAAATCCTAAGGGTAAATGCAATAGAGATAGAACGCAAATAATTGGGGAGGGGGGTTGAC
TGAAATTAAAGATGTATTAATCCAAAAGAAGGCNCAAANTAAANANAANCNNNNNGGTACCTCG
GCCGCTCTANAACTA

| | |
|---|---|
| Sequence 586 | cMhvSH103c09 |

CCGGGCAGGTACTTCAATTGAATCCAGATTTTATTTGTATTTCATTTCTCAATATTTTCTCCTCTACA
AAAACAGAGTGAAGTTGTAAGAATACTAGACCCAAGTTTCAAAATCTCATGTTAAGTGAGATTTTG
CATGTCCTCCGTAAAATTTCTGGAGCACTTTATAAAAGTTTATTTTCGTGGAAATCAAAAAACCAG
GTCATGATATTCTTTTCTAAGTCCCTAAACCTGTCTAACAATGCAAAGGTTGTCTGTCCTTCTTACA
TGTAGACTCATTTGTCTAAGTGGGCCTTAACATGTATGATTTCCATCAAGGCTGCTTGGCAAAGGC
TTTCTGTTAGTGTGTAAGGGGAATATGATGACCAATATAACAACCTCAGTATTTCCTCTACCTCTCT
TCAACTCCTCAACGTGAACCCAATGTTTTTGTGGAACACAAAGCCTCTGAATGCCTGGGAAGTCAC
CAGTGTGATCCCAGCCACCACCCATTAATCTTCTTAACTAGCATGTNCCTCATCATTACCTCCCTTT
CCAAAGCCCTTTGCATGTGCCTGTTCCCTGGCCAGAAAAGCCCTCAACTAAATGGCCCAAGAAGCT
AATGGAGAATTCCCCCCCAAAAATGGGGAAAAATTGGAATATTAAATGGAGAAAAGTTTTAAAAA
GGNNGCCAAAGATCAANGCCCCGGTGCCAGTGGTGGCACCGCCTNGTAATCCCCANCCCCTTTTTA
NAAGGCCCCANGTTGGGGCCGGGNTTAACAANGGTCAGGGAGANTCCGAGAANCCATTNCTTGNG
CTTACAACGGTGAAAACCCTTGTCTNTTACTTAAAAATACCCAAAAAAAA

| | |
|---|---|
| Sequence 587 | cMhvSH106c06 |

AGGTACTTTTTTTTTTTTTTTTTTTGGCCTTATATCAGTTTTATTGGTGGGTTTGTAGCTCCCTGGGC
CGGGCCTGGCTGCTTAGGCCAGTCTCTTGCTCACGCGCTCATAGGTCACGCCTCCGATGGNGGAGA
CCTCCACCAGCTTGTCACCCACGATCTCTGAGGTCTGGTGATAGTTGGGGAAATTCACCACCAGCT
TCCCGCCCTCCATNTGCACAGTGGCCTTAGAACGTCTTGCCCCCTATTGGNCTGTATGTTTGCTTTC
CTTGCCAACAGTGNAACTTGTTTTGGTCATGGNGGTGGCCCCCGGGAGTAGTTGNTTGGGGACCAA
NNTGAAAGTCCTGCCCATCCTTGCTGCACCTTTCCGGTGNACCANTNCTTTGGAAAGTTTNGCCGG
GCCCTTTTTTCNNGAANTACCATCCGNNTTGGGAGGAATCCCCCAAAGGGGAAGNCTNTTCAATG
GAAACCTTCCAATTNCAATAAAATTTTCTTTTCNTCAACNTCTTTCCAATTNTNNNNAAAACTTTGG
CCCNGGGTGGGAAAAAAGCCCAATGGCCTGGNTTGGGGGANNGGCTTTTTCCCTTTTTAATGGTGG
NNCTTGGNTTTTCAATTTTNTTCTNTGCCAANGGTTCTTTCCTTTTTNTCCGGGCTTCNACNCCCATT
GGNGGCNNCCGNCNAAGACCAAAANAANAANNTTTTCCCCCNCGGCCGGTTANCCCTTGCCCCCN
GGGGCGGGGCNCNGCTTTTTAAAAAACTTAANGNTGGGAATTCCCCCCCCCGGGGGGCTTGCNAG
GGGAANTTNCCAATATTNNTAAGCCTTTAATTNCGGATACCCGGGCCAACCCTCTTNAANGGGGG

| | |
|---|---|
| Sequence 588 | cMhvSH106f04 |

GCTCCACCGCGGTGGCGGCCGAGGTACTTATTTTTTTTTTTTTTTTTTTTTTTTTTTNAATTGTTTTTT
TTTTTTTTTTTTTTNCCTGTTTGNCTGATTTTTNTTATTTAAAAAAATGGAAAAACAAANGTGCATTT
TTCATTCAATAAATGNNCCATCCTTATTTAGNTTTGTNNCCNAANGGGAAGTCCNTNNCTTTNGAA
NGGATNTGCAATTTATNAACCANCAGCAATNCNTTTTNACACCGNTTTCAANNAACCTGNNNCNA
NTTTTCCCTTGAACCTGGNGGGGGGGNAAAATTTCTGAAAACTGGGNGGNAGATCNCCCNTTTNA
AAAGCNCCTTTGGGGNCNTTNTACNTTGGGCCCTGAAATNGATTCNNCCCCNCTTTTTTANNNCCA
TTTCCCNTGGAAAACCGTTAAAGGGGNNNNNNCTTTANAAAAAANANNCNTGTCAAAAGNNTNNT
NTTTGNACTCTTNACCAAGGCCNATTANCCCCCAAGGTTTTCNCCNCTTGGGAAAAAATTCTTANN
AAAANCNTGNGGTTNTGGGNGGANCCATTTGGGGGANTTTTANCCATTCCCAGNCGGGNCGGGGN
TTCCCTTTGGNACCCCCNTCCCAATGGGGGCCNCCCGCTTNTTGGGNNAACTTTTGGCGGGCCCCC
GGGAACTTTTTANNAAGACCCCCCCCCNTTTACCCTTNCCCCGGGGCCGGGGCCCGTTTTTTTAAA
AAACTTAAANTNGGNANTCCCCCCCCGGNNCNTGGCGAGGAAAAATTTTTTAAAANTTAAAAGCT
TTTTTTTNNANANCCCCCCNCACNCNTATTAAGGGGGGGGG

| | |
|---|---|
| Sequence 589 | cMhvSH110d05 |

ACTGAAAACCTTGGGATACACCTAAAGCTGCAGTCACAAATTCACAATCCTGAATCTTTTCTTTAA
GAATAAGCAAAAACCAATGCATCTTCAACGTAAACAATGTTAAAGACGAACACAGGCCAGGCACG
GTGGCTCAGGCCTGTAGTCCCAGCACTTTGGGAGGCCAAGGCGGGTGGATCATGAGGTCAGGAGA
TCGAGACCATCCTGGCCAACACTGTGTAACCCCGTCTCTACTAAAAATACAAAAATTAGCCGGATG
TAGTTGGTGTTGCCCCTTGTANTCCCAGCTACTAGGGAAGCNTGAGGCAGGAAGAGTTCCCTTGAA
CCCCAGGAAGCCCGGGAGGGTT

| | |
|---|---|
| Sequence 590 | cMhvSH112g04 |

ACTTTTTTTTTTTTTTTTTTTTTTTTTTTTTCTTTTTTTTTTTTTTTTTTTTTTTTTTTTTTTTTTTTTTTTT
TTTTTTTTTTTTTTTTTTTTTTTTTTTTTTTAAAAAANGGNGNNTNAANAAAANCTNGGNAANANTC
CCAAGGGNAAANGGNAAAANGNNGGGNNANGNNGGNNNAAANGNAAAANNNCGCTTTNNTTTN
CCCCNNCCCNANNAAAAAAAACCNNGGGNAAAANNTNNTAGGTNAAAAAANCAGGNAANCNAN
CATTTNGGGGNCNCNNACGGNAANCCCCCNGGGNGCCCATTNAAAAAAAANGGNANCCCCNGGG
NGGGNGAAATNAANNACAAACTTTTAAANANCCCAANCNCNCGGGGGGGGGNCCCNAANCNAAN
TTTTANNCCCCTTNAANGNGGGTAAATNCCCCCNNGGANAAAAAAAANGGGCAAAAANTNTTCCC
NGGAAAAAAAANGTTNCCCCCAAAAATTCAAAAAAAAAAAAAAACCCNGANAAAAAAAANNTN
AAAAACCCNGGGGGNCCAAAGGGGGGGACCCCNCCNAAAAAAAANTTTGNNTCCAAANCACNCN
CCNNATTTTTCAAAAAAANCNNAAAANACCGTGGTNNNNGCCAAGNTNGAANAAAAAAAAAAAA
ANGGACCACNCCCCCCCGGGGGAAAAANGGGNNNTTNAANAAANNTGGGGGCCCTTATTCCACN
NTTTCTATNAAAAAAAANAAANATCGGGGNGAAAAAGGNAANAAGGGGNGNNGGGGACGGGNT
ATAAAAACNAAACAAAAANGGGGGNAAATNNNNTTTTCCNAANAAAACNAGGGGNAAAAACCCN
AAAAAAAAAAAAATTT

| | |
|---|---|
| Sequence 591 | cMhvSH116f04 |

AGGTACGCNNNANCTTCAGGCTCCGAANCGGTGTGTNGCNGATCNAAGCGCTGNNNGAANNNTN
GANAAACCTNANGAGTAAACNTGTTCCNATCTATGATAAGAACNTGGNCANATCCCCATGTGTGA
CACCGGTGACCAGTGATCATTGAGNAANGGGACANGGATNGGGAAGCTATNTNANTGCCCCNGA
AGAANCTGCTGCANTTCNTTCCTNCTGAANTGCTTATGAAGGGNNNTTACATTCNCCTGCATACAT
TCCCATCCCTCTACTNTCCNCATGAGGACCACACCTTCTCTCCCTGAGAGTTTGGCTTAAGCANCCA
GATNAAGTTTTTTATTTTCNTTTGAAGGGGNAAGGGCTCTTTTCCTGCTNTNTTCGNAAATTAAAAA
NAACCCATTTAGATGTTTANCCGGGNNTAANGAAANAAATGCCNTTGTNTGGGCGGGTTNATNCC
TTGTANTGAAAGGATTTCTNAATTNNTATTTTGGGNANAACAAAAACTTTTTTGNGGTTTNCCTTG
CCCCGGGCNNGGACCNTTTTTAANNNANCTTNTGGGGATNCCCCCNGGGGCTTGNNAGGAAAATT
TTNATTTATNGGAANCTTTTTTTTCGATNCCCGNCNAAANCTTTAANGGGGGGGG

| | |
|---|---|
| Sequence 592 | cMhvSH121g02 |

AGGTACTTTTTTTTTTTTTTTTTTTTTTTTTGAGACAGAGTCTNTNTNTGTTGCCCAGGCTGGAGGGT
AATGGTGCAGTCTCGGCCCACTGCAATCTCCGCCTCCTGGGTTCAAGCAATTCTCCTGCCTCAGCCT
CCCGAGTAGCTGGGATTACAGGAGCCGCTACCACGCCCAGCTAATTTTTGTATTTTTAGTANANAC
TGGGTTTTTCCATGTTGGTCAGGCTGGTCTTGAACTCCTGACCACAGGTGATCTACCCGCCTTGGCC
TCCCAAAGTGCTGGGATTACAGGCGTGAGCCACTGCACCGGGCCTTGGATTTTTGGCATTCTGGAA
TTTTGGCATGGNGGGGGTTCTGGCTGGAGGTGGAANCATCCGTNTTGGCCCCACTGGCCTTGGGGC
CAAAGCCCTGGTCCATCCCCAGGCCAAGTCCTACCAAATCAGCTGCTAAGCCTGAACAAGCACTTG
AAAGCAGGGGTTTGGTCTT

| | |
|---|---|
| Sequence 593 | cMhvSH121g03 |

AGGTACTTTTTTTTTTTTTTTTTTTTTTTTTTTTGGAGGGGGGAGCCTGAAGGTGACATNTTGTTGGT
TTGGAGATGATTTATTCNCTCGTATTGTAAAATCTAAAATGACACTCCTGGGAAGAGGAAGGAACT
ATAAGGACCCGTGTGACCCATTGCTGTCTGCCTGAAGCCCTGGCGCTCTGACCTGAGTGCACCGGG
GTTAGGTGTCTCANCCAAAATGCAGGACTGCACGACGTNTAACACATTGGGANAGATTGCTCTTG
AAACATGGGGGTGGGGTATTCACCTGCATTCCAAAAAGTTTGGGGGGATTCTGGGANACCCCAGT
TGGAGNTCCTTCNGNACTTTCACAAGGGCCTTGTCTTCCCCACACTTTCAAAATTTCCAAANTCGTT
CCTTTNACCCAAAAAGGTGGGGTNAGGGAGTCACCTGGACTATTCAATTTTCCCCAAAAAATTCTT
AAAAAAAAAGGGAGGGTTTACCCCCGGGG

| | |
|---|---|
| Sequence 594 | cMhvSH122e04 |

AGGTACGCGGGGACCGCAGCCCANCAACTCGCAAACGCAACCTGAAGCCTGGGCTGCGCAGTGTG
GGAGGGCTTCGCGATCTTGGGGGACCCATTCCGAACTTGCAGAGGACCGTAGCTCTCCTGGCCTGG
AGAGTGTGAACAGGATTGTGGACTCTTCCAAGATTCACAATGATATGGTGAATCCAAAGACTGGA
ACCAAAAAGATTTACTCAGTGCTTTAGTTTTAACAACAGTAAATTGTCTACCAACACCCATCATGG
CTAAAAGTGCGGAGGTCAAACTGGCAATATTTGGGAGAGCAGGCGTGGGCAAGTCAGCTCTTGTA
GTGAGATTTCTGACCAAACGGTTCATCTGGGAATATGATCCCACCCTCGAATCAACCTACCGACAC
CAAGCAACCATCGATTGATGAAGTTGTTTTCCATGGGAAGATACTANACACTTGCTGGTCAGGGAA
AGATACCATTCAGAAGGGANGGGGCACATGCGATGGGGGGGAANGCTTTTTGTGCCTGGTCTTAC
NACATTACTGACCGANGAAGTTTTTTGAGGAAANTGCTTCCCACTTAANAAAACATTCTTANANTG
ANGATCNAAAAAAGCCC

| | |
|---|---|
| Sequence 595 | cMhvSH124b09 |

ACTTTTTTTTTTTTTTTTTTTTTTTTTTTTTTTTTTTTTTTTTTTTTTTTTTTTTTTTTTTTTTTTTTTTTTT
TTTTTTTTTTTTGCCCGGGGNAANCANNNTTTTTTTTAAANCNANANTTNAAAACTTTTANTTTTNG
NANNAAAAANNNGGGNNTTTTTAAAAAANNGGGNAANCCNNNANAAAATTTTTTAANTNTNAAN
NNNTNNNTTTTTTAANTTTTTTCNNNANTNNTTCCCAAAATNNGNTTTTTTTTTTAAANNNAANTTT
AANCCNGNNNTTTTTCNNCNCNAAANTGGGGNAAAAAAGTTTNNGGGGGGGNAAAAANTTNGGN
NGNNNTAAATTNAAAAAGNGNTTNTTTTTTNAAAAAAAATTTTAANNCNTTAAAAAAAAAACNGG
GGGAAAAATGGGGTTTNGCTTNNTAAAAAAAAANGGCCNCNGTNNCCNACNNNGGAACCCCCCN
CCNCCTTTANNGGGGNNTTTTTTTTNNTNGNNCCCTTTCTTTTAAAAAAAAANAGNGNNGTTTTGG
ANNCCCCCCAANNGNNNNCCNCCCCNAACCTNGGGNCCTTTTTAAAANNTNGNGGGNTCCCCCCG
NGGNNNNNNAAATTTTTTTTTTNAAGNTTTTTTTTTTCCCCTTTACNTTTTTNGGGGGGGGCCCNGG
GCNCCCNAANTTTTTTTTTCCCTTTTTTGGG

| | |
|---|---|
| Sequence 596 | cMhvSH124f10 |

CCGGGCAGGTACCGGGATCGCCGAGACAAGGTGGCAGCAGGTGCTTCNGAAAGCACACGGTCAA
ATGAGAGGACCGTCATTCTGGGAAAGAAAACAGAAGTGAAAGCCACNAGGGAGCAAGAAAGAAA
CAGACCAGAAACCATNCGAACAAAGCCAGAANAGAAAATGTTCGATTCTAAAGAGAAGGCTTTCG
AGGTAGAGAAACCTAAGATGGGAAGAATTGACNAAGTTAGATNAAGGAAGCCGAGACNNNAANA
GAAAGCCCANCCAGATGAAGGGAGAAGGGCTAAGGGAAGAAAGGACTNCACCCNGAAAGGGAN
AAAGAACCGTTGCCNAAGAAGAANAAGAGGGTGCCCCGATTTAGTNTTAGAAAGGTANTCCCCCA
GGGACAAGAAAGAAGCCAAGGAAGGGTGTTCCCCCCNTAAAA

| | |
|---|---|
| Sequence 597 | cMhvSH126a03 |

TTTTTTTTTTTTTTTTTTTTTTTTTTTTTTTTTTTTTTTTTTTTTTTTTTTTTTTTTTTTTTTNGGGNCCNN
GGGGAAANTTTTNTTTTTNCCNNCNGNANCNANNNTTTTNCNAAANCCNGNACCCCNGNNTTNGN
NAAAANCCNGNAAANNTNNNTNTTTTGCAAAAAAAAAATNNCNNCANGNCNNNCCTTTNCNNTTT
GNAANTCCNTTNNGCCNNAANTTAANCNCCTTNCCCATNGGGGCANNCCTTTAANGAANNTGGNG
GTTCTNCTTNNNCCCCTGGGGNAAAAAAAAGGGGGNNNTTCNGGGGNAGGGGGGGAAAAANAC
AACNCNTGGGGGGGGGGNTTTNAAAAAGGCCCCCCCNNNCCANNNNANNNTNANNCCCCTNTNG
GGGGGAAANTNACANANNTNTTTCNTGGGGNGNCCCCAAAANNCCTGTGNCGNCNNNANGATTTT
GGAGGGGTNCTTTTTTTNTCNNGACCCCNTNAACATNNAGACNNGGNTTTGGGTGANCCCCCCGN
CCCTNTTTTANNTTNTTCTCNNCCCCCNGGGGGGGG

| | |
|---|---|
| Sequence 598 | cMhvSH127f12 |

AGGTACAAACCCAGTTTGTTTTCAAAAAATCACAGTAGCAATGCAACTCATCACTCTAGAAAAGC
AAGCTTAGGCTACCTGAAAGATTTTCCCTTGGAAGTTTAGCGTATGTTTGACTAACAAGAATTCCC
TACATCAGAGACTCTAGGTGCTATATAATCCAAAAACTTTTCAGCCTGTTGCTCATTCTGTCCCATG
CTGGCAATAATACCTTGTCAGCCCATTACCCTTATTTTGAATTGCTCCATCTCCTGGTGGGACTTGT
ATCTTGTCTGCCATATCAGAACACAAACCCCTGAAGAGGTTCTGATTTTGATTTTTTTTTTTTCTTCA
TGCCTACCCTTTTTTTGGAAGTTTCCAGCCGCAATTTNAAATGAAATGACAAGGTGTATATTTGATC
AATTTTCATTCCCACCATTGCATTCAAACCTCTAACTTAAATGGGTAACCCTAAGGCATATNAAAA
GAANCAGACTGCATGGATAAAAACGGGAAAATAGAAAAAAAAAGGAACCTTACCATTTAATTTTT
GGGTTTTAAGCAACCNTTTACTTNTCACNTTTTTATGGAANAATTNGAGAAGNTGGGACCTTTACC
ATTTTCCCTTTTTTTTAACATTTTNTCGGAATTNCTTTTATTTTTTTTTTTT

| | |
|---|---|
| Sequence 599 | cMhvSH130h08 |

CTCATATAGGCGAATGGACCTCCACGCGGTGGCGGCCGCCCGGGCAGGTACTTTTTTTTTTTTTTTT
TTTATTATANAAAACAAGTGAGGNCCNAATGATCACAAAAANAAGGAATAATTCTAAGTCTCAAA
ATTGGCAAGAAATAANGTCNGATGCTAAAGTCCAAANNTTACGATAATGCACTTGNGCCAGGACC
AATGCCNATANAGAACTTGAAAATTAAGATGAGACATTTTTNAAGAACAAGTGA

| | |
|---|---|
| Sequence 600 | cMhvSH005c02 |

AGGTACTTTTTTTTTTTTTTTTTTTTTTTTCGAGATGAAGTCGCTCTGTCACCCAGGCTGGATGGAGT
GCAGNGGTACAATCTCAGCTCGCTGCAACCTCCGCCTCCCAGGTTCAAGCGACTNTCCTGCCTNAG
CCTTNTGAGTAGCTGGGATTACAGACCCATGCCAACACGCCCTCCAATTTTTGCATTTTTTTTTGTA
NANACAGAGTTTCACCATGTTGGCCCAGCTGGTCTCGAACTCATGACCTTGTGATCCGCCTGCCTC
GGCCTCCCAAAATGCCGGGATTACAGGTGTCAGCCACCGNGCCTGGCCTTATTTTCATAGTAATAT
GTAAAATATCCATAATGNGATCAACTGNGTATTTATAATAAATTTTAATAATATCTCCGTAA

| | |
|---|---|
| Sequence 601 | cMhvSH014d04 |

GGCGAATTGGAGCTCCCCGCGGTGGCGGCCGAGGTACTTTTTTTTTTTTTTTTTTTTTTTTTGGGACG
GAATTTCATCCAGGCTGGAGTGCAATGGCGCAATTTTGGCTCACTGCAACGTCCGCCTCCCATGTT
CAAGCGATTCTCCTGCCTCAGCCTCTCGGGTAGCTGGGATTACAGGCATGAGCCACCATGCCCGGC
TAACCTTGTATTTTCAGTAAAGATGGGGTTTCTCCATGTTAAGAATTGAGAGAGCCACTGAAAGGN
GAGTCAGGAAGCNTCATGATCACAGCCGTGCCTTA

| | |
|---|---|
| Sequence 602 | cMhvSH051a12 |

TCTGTCTCCCAGGCTGTAGTGCAGTGGCATGATCACGACTCACTGCAATCTCTGCCTCCTGGATTCA
AGCAATTCTCCTGCCTCAGCCTCCTGAGTNGCTGGATTACAGGCACACACCACCACGCCTGGCTAA
TTTTTTGTATTTTTGGTAGANATGGGGTTTCAACATGTTGGCCAGGCTGGTCTCAAACTCCTGACTT
CAAGTGATCTGCCTGCCTCAGCCTCCCAAAATGCTAAGGTTGCAGGCGTGAGCCACCGNTCCCAGC
CTNAAAATAGTTTCTAATGATNGGATACATCCAGTTCTCCANATCCAGCATTCTGGTTACTTAACA
AAGAGATAATAGTTTCTTTTATTGCTTCT

| | |
|---|---|
| Sequence 603 | cMhvSH070e02 |

ACCTGTAATCCCAGCTACTGGGGAAGCTGAGGCAGGAGACTCGCTGGAACCCAGGAGGCGGAGGT
TGCAGTGAGCTGAGATCTCACCACTGCACTCCAGCCTGGGTGATGGAGCAAGACTCCATCTCCAAA
AGAAAAAAAAAAGAGAGGCCCCAGTTCAGGCTAGCTCTGTCTGTCTTGTGGGGCA

| | |
|---|---|
| Sequence 604 | cMhvSH091f06 |

TTGGAGCTCCACCCGCGGTGGCGGCCGAGGTACTTTTTTTCTTTTTTTT

| | |
|---|---|
| Sequence 605 | cMhvSH093c03 |

CCGGGCAGGTACTTTTTTTTTTTTTTTTTTTNNANTAAAGGGGNTTTTTTTTTTTTAAAANNANNNN
AAAAAANCCNTTTNCNTTNAAAANAAAAAAAAAA

| | |
|---|---|
| Sequence 606 | cMhvSH112e09 |

CCGCGGTGGCGGCCGCCCGGGCAGGTACTTTCTCTTTTTTTTTTTTTTTTTTTGAGAGATAGAGCC
TCACTNTGTCACCCAGGCTGGAGTGCAATGGCATGATCTTGGCTCACTGCAACCTNCGCCTCCCGG
GTTCAAGCCATTCTCCTGCCTCAGCCTCCCAAGTAGCTGGGATTACAGGCACACGCAACCACGCCC
AGCTAATTGTTTTTGTATTTTAGTAGANATGGGGTTTNACCATGTTGCCCAGGCTGGTCTTAAATTC
CTGAGCTCAGGCAATCCACCCGCCTCANCCTCCNAAAGTCCTAGGATTATAGGCGTGAGCCANCA
CACCCNGCAAGA

| | |
|---|---|
| Sequence 607 | cMhvSH091c09 |

ATTGGAGCTCCCCGCGGTGGCGGCCGAGGTACTTTTTTTTTTTTTTTTTTT

| | |
|---|---|
| Sequence 608 | cMhvSH104d01 |

CCGCGGTGGCGGCCGAGGTACTTTTTTTTTTTTTTTTTTTTTTTT

| | |
|---|---|
| Sequence 609 | cMhvSH041a04 |

CGGCCGGAGGCTGACGAGAGCCGGGAGGCGTTAGCAGAAGGAAGAGAAAAACCNAAGACTAAGC
CACTACAGCGNCNCACCGCGGCGCGGCAGTCTGNTTTATAGGAGAGGGCGCANGCCCNCGNGTAC
CTNGN

| | |
|---|---|
| Sequence 610 | cMhvSH041a04 |

CGCTTGGCGNTAATCATGGTCATNAGCTTGTTTCCTGTGTGGAAATTGNTATCCCGCTCACAATTTC
CACACAAACAATACCGANGCCCCGGGGAGCATAAAGTGTAAAANCCTGGGGGTG

| | |
|---|---|
| Sequence 611 | cMhvSH094h05 |

AATTGGAGCTCCCCGCGGTGGCGGCCGCCCGGGCAGGTACTTTTTTTTTTTTTTTTTTTTTTTTTNGG
GNNCCNTTNNTNNAAAAACCNNNGGNCNAAANGGNTTTTNANGGNTTTAAANNNAAAAANCCCN
TTTTTTTNCNTTTTTNNCCCCCNNNNTTNAAAAAAAAAAAANTTTTTAAAANNTTTTNGGNAAAAN
NNNNNNNNTTTTTAAAAAAANTTTTTTNNNNCNGGCCCCCCCNCGGGANTTTTTTTTTTTTTTTTTA
AANGGGNTTTTTTTTTAAAAAAAAANNTTTTNCCCNNTTTTNNTTNANGGGGGNTNANNCCCCCCC
CNNTNNNNGNAANCNTNNTTTCCCCCNAAAATTNGNCCCANAAAAANNCCGGGGCTTTNGGGGGT
TTNTNGGGGGGNAAAATTTTTTTTTNGNAANCCAAAANTTTTTTTTNANGGTNNAAAGGCCCANTT
TTTNGGGNAAAAAAAAACCCCCCCNTANAAAAAAAANAATTTTTTTAAAANAAAAAANGGCCCCT
TTTTAANTNTNAAAAAAAAAAAAANANGGGGAAAATTTNTTTNTTTTNGGGGGAAAANGGGGGG
GTTTTTCCCCCCAAATTTTNNAAAAAGGGNGNGGGAAAAACCCCCNGNTTAANNTNGGGNCNTTT
TTAAAAAAANAGGNGGANCNCCCCCGGGGCGGGGANAAATTTTNANTTAAANTTTTNTNNANCCC
CCCCCNNC

| | |
|---|---|
| Sequence 612 | cMhvSG038d04a1 |

CATCTTGGTCCTTTTCCACCATTTTCAGCCCCTCCAGGGCTGGGAGGACCCGGNANGANNANACTC
TTNGNNCCTCGGCTGAAGTGGCTGGGCATGACGCCGTTTCTCTGACGTCCCCCATAGATCTTGGTC
ATGGAGCCAACCCCAGCGCCACCCCGGAGGTACCT

| | |
|---|---|
| Sequence 613 | cMhvSG038d04a1 |

TAGTGAGNGGTTAAATTGCGCCGCTTNGGCGTNAATCATGGGTCCATAAGCCTGNTTTTCCTTGTT
GTGAAAAATTTGTTTATTCCCGCTCACAAATTCCACCACCAACAATACNGAAGCCCGGGGAGGCAT
AAAAAGTGTNAAAAGGCCTTGGGGGTGCCCTTAATGGAGTGGAGCTAAACTCACATTTAATTTGC
GTTGGCGGCTCACTTGCCCGGCTTTTCCCANGTTCGGGGAAACCTTGTCCGTGGCCAANCTTGCCA
NTTAAATGGAAATCGGCCCAACGCCGCGGGGGGAAGAAGGCNGGTTTTGCGTATTGGGGCGGCTC
TTCCCGC

| | |
|---|---|
| Sequence 614 | cMhvSG025b07a1 |

GGGCAGGTACTACNCAGGCCTTGGCATNCCTGGGGTTCACCTGGCTGACTGGGGTGTTTGAGGCG
GGCAGCAATGTCTTCCACGGTCTCATTGCCTTCTGAGATGATGCCCACACCTTTGGCAATAGCTTTA
GCTGTGATTGGATGGTCTCCTGTGACCATGATGACCTTAATTCCAGCACTTNGACATTTGCCCACG
GCATCAGGAACGGC

| | |
|---|---|
| Sequence 615 | cMhvSG025b07a1 |

CGTCGACCTCGAGGGGGGGGGCCCCGGTACCCAGCTTTTTGTTCCCCTTTAGTGGAGGGGTTTAAT
TGCGCCGCTTTGGGCCGTTAATCATTGNGTCATAGCATGTTTTCCTGTGGTGGAAAATTTGTNTATC
CCGGCCTTCACAAATTTTCCCACCACCAAACCATTACCGAAGNCCCGGGGGAAGGCCATTANAAA
GNTGGTANAAAGGCCCTTGGGGGG

| | |
|---|---|
| Sequence 616 | cMhvSG048d02a1 |

CCGGGCAGGTACCATTCGCACACAGAGATATCGCCTNCTTTAGCGGTCATTGCCTTCTGACAGCGG
TGGAAGTCCAGGTAGTTCTGCCAGCAGTTTCTAGTCTGGTTCTGGTTGGGGAAGCGGCTGTCAAAA
GGGGCGGTCTTGTAGTTCTTGATTTTGGTCTCCATGTCTTCCGCCATGGNGCTGAATCCTAAAGGCA
CCCCGGATTCAACCTGCAGCTCAATGTGGACCCTCAGCAAAGACACCACAGTCGGACAGGAAGCG
GAAACTACTACCAGCCCGGAAGCTGANAGAGGTGGGGACTACCGGGNAGTCTCCCCGCCGTACCT
CGGCCCGCTCTAGAAACTAGNGGGATCCCCCGGGCTTGCAGGAAATTCGATATCAAAGCTTATTCG
GATACCCGTCNGACCTCGAGG

| | |
|---|---|
| Sequence 617 | cMhvSG048d02a1 |

TAANTGAGGGGTTAAATTGCNCCNCTTGGGCCGTAANTCAATGGTCCATAGCTGTTTTCCTGGTGT
GGAAAAATTGTNTATTCCCGCTTAACAAATTTCCCACACCANCCATTACCGAAGCCCGGGGAGCCA
TTAAAANGTNGNTAAAAAGCCCCTGGGGGGTGGCCCTTAAATTGAAGGTNGGANGGCTTAAACTT
CACCATTTTAAATTTGCCGTTTGGCGCCTCNACTTGCCCCGNTTTTTTTCCNATTNNGGGGGGAAAA
CCCTTGTTGCGNTGNCCCAACCTTTGCCATTTTAAATTGAAAATTCGGGCCCCAANCCNCCC

| | |
|---|---|
| Sequence 618 | cMhvSG070a01a1 |

CCGGGCAGGTACCTCAGTCCACATCTCCTTCACGTTCTNCAGNGNNCATGTTGCAGCGCCTATCGA
AGGCCTTCACGCGGCCCAGGAGTTTCTTATTGTTGCGGCAGTTGATGAGCACTTGGGTATTGTTCTT
GACTGACTGTGTGAGCACAGAGAGTGGACCGGTGTTAAATTCCTCCTCCTCTCGCTTCTGCAAGCT
CCTCTGGGGTCATCTCACTNTTGGGCTTGTTGAGGAGGCTCATGATGGTCACTACGCTCTCCGTTCA
CTCCCGTTTCCTCCCCCGCGGTACCTCGGGCNCGCTCTAAGAACTTAGGTGGGATCCCCCGGGCCT
GCAAGGGAATTCCGATATTCAAGCTTATCGATACCCGTCGACCCTTCGAGGGGGGGGGGCCCCGG
GTACCCAAGCCTTTTGTTTCCCTTTTAAGTGGAGGGTTAAATTGCGCGCTTGGCNGNTAAATCATG
GGTCANTAGCCTGTTTCCCTGTGTTGAAATTTGGNTNATCCCGCTCACAATTTCCANCACAAACATT
ACGAAGCCCGGGGAGCATAAAAAGTGGTAAAAAGCCTGGGGGGGTGCCTTAATGGAGGTTGAAG
CTTAAACTTCACAATTAAAATTTN

| | |
|---|---|
| Sequence 619 | cMhvSG071h12a1 |

GGAGCTCCCCGCGGTGGCGGCCGCCCGGGCAGGTACGCGGGGACATTTTCTCGGCCCTGCCAGCC
CCCAGGAGGAAGGTGGGTCTGAATCTAGCACCATGACGGAACTAGAGACAGCCATGGGCATGATC
ATAGACGTCTTTTCCCGATATTCGGGCAGCGAGGGCAGCACGCAGACCCTGACCAAGGGGGAGCT
CAAGGTGCTGATGGAGAAGGAGCTACCAGGCTTCCTGCAGAGTGGAAAAGACAAGGATGCCGTG
GATAAATTGCTCAAGGACCTGGACGCCAATGGAGATGCCCAGGTGGACTTCAGTGAGTTCATCGT
GTTCGTGGCTGCAATCACGTCTGCCTGTCACAAGTACCT

| | |
|---|---|
| Sequence 620 | cMhvSG071h12a1 |

CCCTGGGNGGGGGGGGCCCNCNNCCAAGTTNNNGTTCCTTGGGGGGNAGGGTCNCCNCGCCCCTT
GGCCNNNAAAAAANGGNTTTTCCTTTTNNGTNAAAAAGNGAAAAANNNGNTAAAAAANTTCAAA
AAAAAAANAAAAAAANNNNGGGNNNNNAAAGANAAAAAAAAAACGGGGGGGCCCCNNANGGG
GNNNNNNNNAACCCCCCNNTTNTTTTTNTNNTNGTNTCTTCCNCCCTNTTTTTTNNGNNAAAAAAA
AAANANNGNCCCCCCCTNTTTTTTTTTTNNTNNTTNCCCCCCCCCCCNCGGGNNANNNNGGGGGNN
NNNNNTTTTTTTGGGGGGTTTTTTNNNTTTTTTTTNNAAAAAAAAAAAAAAANNNNNNNNNNNNN
NGGNGGGGGGGGGGG

| | |
|---|---|
| Sequence 621 | cMhvSG039e01a1 |

AGGTACGCGGGGGTGTCCGCACAGAGGTCTGCAAGGAGAGAGAGTGTCTTCATTCTTTCCGCCATC
TTGATTCTTTCTCACTGACCAAGACTCAGCCGTGGGAAATATGAGTGAGCTTGTAAGAGCAAGATC
CCAATCCTCAGAAAGAGGAAATGACCAAGAGTCTTCC

| | |
|---|---|
| Sequence 622 | cMhvSG078d09a1 |

AGGTACTCCCCAGCAAATATTCTTTGTTGGCTTGCTTGACTAGATGAGCTGCTATAGTAGTCAATCC
TGTTAGACTTGGACCATTGTTTGTCTGAAGAACTGGAATCTGTCGCTCGCCCTGAGCACTGTATTTA
TTCCCCTTACTCAGTCCCAGGGACTTCTCCAGTAGCGACAACTCT

| | |
|---|---|
| Sequence 623 | cMhvSG078d09a1 |

CGATACCGNCGGACCTNCGAGGGGGGGGCCCCGNGTACC

| | |
|---|---|
| Sequence 624 | cMhvSG078d09a1 |

AGGGGTTAATTGCCGCCGCTTGGCGTAAATCATGGGTCATTAGCCTNGTTTCCTGTGTGAAATTGG
TTATCCCGCTCACCAATTTCNCACACAACCATTACGAAGCCCGGGGAAGCCATAAANGTGTANAA
AGCCCTGGGGG

| | |
|---|---|
| Sequence 625 | cMhvSG027c01a1 |

GGGGCAGCTGGAGGTGCCTCAGAANGTGCATTCTGCTTCCTGCAGGGGCTTGAAACACCAAGGCA
CTCCAGGGATCCTGGAGTCAAAGCAGCAGCCCCGGTTGTTGCACTCCTTGGGGGTGACATGGGGG
TAGCCGCAGTCCACCCTGTCCTTGGCTGGCACGGCACACTGGTTTGCAGACAGGCCCACGT

| | |
|---|---|
| Sequence 626 | cMhvSG027c01a1 |

TACCCAAGCTTTTGTTCCCTTTTAGTTGAGNGGTTAAATTGGCGCCGCTTTGGGCGGTAATCATGGG
TCATAGNTTGTTTCCTGGTGTGAAATTGTTATCCCGCTCACAATTCCACACCAACATAACGAAGCC
CGGNGAGCATAAAAGTTGTAAAGCCTGGGGGTGCCTA

| | |
|---|---|
| Sequence 627 | cMhvSG055b12a1 |

ACTTGCCCCAAATGTGCAACATAAATACAGAAGCGATGAACAGAAGACTCATAACCAATACTGGA
ACAGGGCCAACTTTGAACCCAGGTGAATCTTCTGTGTAGAATCGCCACATCCCCCCGGTGCCTGCC
GAGGTTGTGCGGCCTGCACTCCTTGTCCCACAGCTGGCATTTTTCCTCTGCCGGACAGTGGATCCC
GCC

| | |
|---|---|
| Sequence 628 | cMhvSG055b12a1 |

TGTTCCCTTTAGTGAGGGGTTAATTGCCGCGCTTGGGCCGTTAATCATGGTCAATAAGCCTGTTTCC
TGTGGTGAAAATTGTTATCCGCTCACAAATTCCACAACAACATACGAAGCCCGGGGAGCATAAAA
AGTGTAAAAGCCTNGGGGTGCCCTAATGGAGTGGAGCCTAACTTCACATTAAATTGCGTTTGCGCT
TCACTGCCCGCTTTTCCAAGTTCGGGGAAACCTGTNCGTGCCAAGCTGCATTAATTGAAATCGGCC
CAACGCCNCGGGGGAGAAGGCGGTTTTGCGTATTTGGGGCGCCTCTTCCCGCTTCCTTCGCTTCAC
TTGGACTTCGCTGGCGCCTCGGTCCGTTCCGGCTTGCAGGCGAGCCGGTNATTAAAGCTTCACTTC
AAAAGGGCGGGGAAANTAACNGNTTTNTNCACAAGNAATCNAAGGGGGGATTAAACCGCCAGGG
AAAAAANAAANATTGTTNAANNCNAAAAAAGGCCCAGCNAAAAAGGGCCCATGGAAACCCGTNA

| | |
|---|---|
| Sequence 629 | cMhvSG045h05a1 |

AGGTACGTCCAAATGACGAAGTCACTGCAGTGCTTGCAGNNCAAACAGAATTGAAAGAATGCATG
GTGGTTAAAACTTACCTCATTAGCAGCATCCCTCTACAAGGTGCATTTAACTATAAGTATACTACC
TGCCTATGTGACGACAATCCAAAAACCTTCTACTGGGACTTTTACACCAACAGAACTGTGCAAATT
GCAGCCCGTCGTTGATGTTATTCGGGAATTAGGCATCTGCCCTGATGATGCTGCTGTAATCCCCAT
CAAAAACCAACCGGTTTTATACTTATTGGAAATCCTAAAGGTAGGAAATAATGGGAAGCCCCTGT
CTGTTTTGCCCACACCCCAGGGTGGATTTTCCTCTTAAAAGAAAACCTTGGGCTGGGAATTTCTGG
CTGTGGGTCTTATTAAAATAAAACCTTCTTTAACATGGCTTCCCCGGANGNAAANAAAANANCTTN
NNATANNCANAATTAAAAAGGTACCCTTNNGGGCCCGGCTTCTTANNAAACCTAGGNGGGGATCC
CCCCCGGGGCCTGGCAAGGGAAATTTCCGAATNNTTCAAAAGCCTTTATTCCGATNANCCGGTCGG
AACCCTCNNAAGGGGGGGGGGCCCCGGGTTANCCCCANCNTTTTTGG

| | |
|---|---|
| Sequence 630 | cMhvSG027b03a1 |

CCGGGCAGGTACCCTTTCCAAGGTGACCTTCAGGGGGATTAACCTTCCTAGCTCAAGCAATGAGCT
AAAAGGAGCCTTATGCATGATCTTCCCACATATCAAAATAACTAAAAGGCACTGAGTTTGGCATTT
TTCTGCCTGCTCTGCTAAGACCTTTTTTTTTTTTTTACTTTCATTATAACATATTATACATGACATTA
TACAAAAATGATTAAAATATATTAAAACAACATCAACAATCCAGGGATATTTTTTCTATTAAAAAC
TTTTTTAAAAAATAATTGNATCCTATTATAATTCAATTTTTACATCCTTTTTTCAAAGGCCTTTTGTT
TTTTCTAAAAGGGCTTTGGTTTNTCCTTTTTTATTATTTTTTTGTCCTTTTTTATTNTTTTTTGGAGGA
CAAGTCTTGGCCTTCTGTTCCGCCTTCAAGGGCTNGGGAGTGGCAAGTTGGGCCACCGAATCCTTC
AGGCTTCAACCTGGCGAAACCCTTCCCTTCCCTTTCCCAGGGTTTNCAAGGGNGGAATTTCNTTTN
GTTTCAATTCAAGACCCCTCNCCCGAANTTAGGCCTTGGGGGACCTTACCAAGGGCCATTGGTGGC
CCACCTTNTTGGCCCCCAGGGCCNNAAATTTTTTTTGGTGANCCCTCNGGGNCCCGCNTTCTTANG
AAAACCTTAANTGGGGAATCCCCCCCCCGGGGGCCTTGGCAGGGNAAANTTTCNGNTTTTTCCAG
ANGCCTTTTNTTTGGATTACCCCGGTNCGGANCCCTTCNGAANGGGGGGGGG

| | |
|---|---|
| Sequence 631 | cMhvSG025b08a1 |

AGGTACTGATGCAACAGTTGGGTAGCCAATCTGCAGACAGACACTGGCAACATTGCGGACACCCT
CCAGGAAGCGAGAATGCAGAGTTTCCTCTGTGATATCAAGCACTTCAGGGTTGTAGATGCTGCCAT
TGTCGAACACCTGCTGGATGACCAGCCCAAAGGAGAAGGGGGAGATGTTGAGCATGTTCAGCAAG
CGTGGCTTCGCTGGCTCCCACTTTGTCTCCAGTCTTGACCCGCGTACCTGCCCGGGCGGCCGCTCTA
GAACTAAGTGGATCCCCCCGGGCCTGCAAGGAAATTCGGATATCAAAGCTTATCGGATACCGTCC
GACCTCGAGGGGGGGGGCCCGGGTTACCCAAGCCTTTTTGTTCCCTTTTAGTGGAGGGGTTAATTG
CGNCGCTTTGGCGTTAATCAATGGGTCAATAGGCTGTTTTNCTGTGGTGGAAATTGGTTTATCCCG
CTTCACAAATTTCCCACCACCAAACATTACGAAGCCGGGGAGGCCATTAAAAAGTGNTAAAAAGC
CCTGGGGGTGCCCTTAATGGAAGTGGAGNCTAACTTCACCATTTAAATTTGGCGNTTGGCGCCTTN
AANTGGCCCCCGGCTTTTTTCCAAGTTCGGGGGAAAAACCTTGGTCCGGTGGCCCNAANCNTTGGC
ATTTAAATTGGAAATTCGGGCCCCAAAACGCCNCCCNGGGGGAAGAAGGGCCGGGT

| | |
|---|---|
| Sequence 632 | cMhvSG024g12a1 |

ATAGGGCGAATTGGACTNCACCGCGGTGGCGGCCGNCGGGCAGGTACGCGGGGGACTTAGTGCTC
ATGCTCGCTGCAGGGGTCGGAGGTCAGGGCGAGCGTCTNGCAGGCCGTAGGAGGAAGATGGCGGT
GGAGTCGCGCGTTACCCAGGAGGAAATTAAGAAGGAGCCAGAGAAACCGATCGACCGCGAGAAG
ACATGCCCACTGTTGCTACGGGTCTTCACCACCAATAA

| | |
|---|---|
| Sequence 633 | cMhvSG024g12a1 |

CGACCTNGAGGGGGGGGCCCCGGTACCCCAGNCTTTTGTTCCCTTTTAGTGGAGGGGTTAAATTNG
CGCGCCTTGGGCGGTAATCATGGGTCATAAGCTGTTTTCCCTGTTGTGGAAAAATTGTTATCCGCTC
ACCAATTTNCANCACAAACAATACGAAGCCGGGGGAGCCATTAAAAAGTTGTTAAAAGGCCCTTG
GGGGGT

| | |
|---|---|
| Sequence 634 | cMhvSG043g05a1 |

TCATCCCTCTACAAGGTGCATTTAACTATAANTATACTGCCTGCCTATGTGACAGACNATCCAAAA
ACCTTCTNCTGGGACTTTTACACCAACAGAACTGTGCAAATTGCAGCCGTCGTTGATGTTATTCGG
GAATTAGGCATCTGCCCTGATGATGCTTGCTGTAAATCCCCATCAAAANCAACCCGGTTTTTTATA
CTATTTGAAATCCCTAAAGGTTAGAAATAAATNGGAAAAGCCCTGNTCTGTTTGCCCACCACCCCA
GGTTGGATTTTTCCCTCCTNAAAAGNAAAACCTTGGGGCCTGGGGAAATTTTCCTNGCCTGGTAGG
GTCCTTATTAAAAAAAATAAAAAACCTTTTCTTTTAAACCATTGGCCAGANTATGNNCATAGTGAA
TTNNNCGANTNTNCNTAAATATTNNTNNTNTNGGNTTCCCNTTNGGGCCCCGGNTTCTTAANAAAC
NTATTTTGGGNAATCCCCCCCCGGGTCNTGGCNANGGNAANTTTCGGATATTCNAAAGCCTTTANT
CNAGATTACCCGGGNCCNNACCCCTCATAAGGGGGGGGGGGCCCCGNGG

| | |
|---|---|
| Sequence 635 | cMhvSG048f11a1 |

ATATAGGGCGAATTGGACTCCACCGCGGTGGCGGCCGCCCGGGCAGGTACGCGGGGGCAGTTCGG
CGGTCCCGCGGGTCTGTCTNTTGCTTCAACAGTGTTTGGACGGAACAGATCCGGGGACTCTCTTCC
AGCCTCCGACCGCCCTCCGATTTCCTCTCCGCTTGCAACCTCCGGGACCATCTTCTCGGCCATCTCC
TGCTTNTGGGACCTGCCAGCACCGTTTTTGTGGTTAGCTCCTTCTTGCCAACCAACCATGAGCTCCC
AGATTCGTCAGGAATTATTCCACCCGACGTGGAGGCAGCCCGTCAACAAGCCTGGTCAATTTGTAC
CTTCGGGCCGCTCTTAGNAACTAAGTGGATCCCCCGGGNCTGCAGGGAAATTCCGATNTCAAAGCT
TATCCGATACCCGTCCGACCTTNGAGGGGGGGGGCCCCGGTACCCAAGCTTTTTGGTTCCCTTTAG
TGAGGGTTAAATTGCGCCGCTTGGGCGGTAAATCATGGTCATAAGCTGTTTCCTGTGTGAAAAATT
GTTATCCCGCTTCACAATTTCCCACACAAACCATTACCGAGCCCGGGGGAAGCATTAAAAGTGTTA
AAAGCCCTGGGGGGTGGC

| | |
|---|---|
| Sequence 636 | cMhvSG045a12a1 |

AGGTACTTTTCCCCACACCAGCGGTGCCGACTACCACGACGCGGTAATCTCTGATCTTCCTGTGGG
GCTTGAAGGCGCGGAGGATAAGCAGGGCGGGCAGAAGCCGCAACCGCTTCAGCAGCTTCTGTTCC
TTGGAGCCAAAGCTGGCGTTACCCATCGTTGGGATTCGGAGGGGAGATACGTGCACAAGTTCTCCC
ACACTTAGCTGGCAGCAGGAGACCCCTTTCTCGGAGGCACGAACCAAGCAGCCTTAGAAGACAAA
TGCGCTGCTCGGAAGAGACTGCCGCGGCAACCAACTGGGACACCCCCCGCGTACCNTGCCCGGGG
CGGCCCGCTTCTAGAAACCTAGTGGGATCCCCCGGGGCTGCAAGGGAATTTCGATATCAAAGCTTT
ATCGATACCCGTCGACCTCCGAGGGGGGGGCCCGGTTACCCCAGCTTTTTGTTC

| | |
|---|---|
| Sequence 637 | cMhvSG011e09a1 |

AGGTACCTGCAGGCCTCCCACACCTACCTCTCTCTGGGCTTCTATTTCGACCGCGATGATGTGGCTC
TGGAAGGCGTGAGCCACTTCTTCCGCGAACTGGCCGAGGAGAAGCGCGAGGGCTACGAGCGTCTC
CTGAAGATGCAAAACCAG

| | |
|---|---|
| Sequence 638 | cMhvSG011e09a1 |

GTTAATTGCGCCGCNTGGCCGTAATCATGGGTCATAACTTGTTTCCTTGTGTGAAATTGGTATCCCG
CTCACCAATTTCCACACAAACATACCGAAGCCCGGGGGAGCCATTAAAAGTGTAAAAGCCTGGGG
GTGCCTAATGGAGTGAAGCCTAACTTCCACATTTAAATTTGCGTTTGCCGCTTCACTTGCCCNCNTT
TTCCAANTCCGGGNAAAAACCCTNGTNCGTGGCCCAAGCTTGNAATTTAAATNGAAATCCGGGCC
CAACCGCCC

| | |
|---|---|
| Sequence 639 | cMhvSG055f10a1 |

GGTGGCGGGAGGAACCGTTACGGGAACTGAAGTTGCGGATTAAGCCTGATCAAGATGACAACCTC
CCAAAAGCACCGAGACTTCGTGGCAGAGCCCATGGGGGAGAAGCCAGTGGGGAGCCTGGCTGGG
ATTGGTGAAGTCCTGGGCAAGAAGCTGGAGGAAAGGGGTTTTGACAAGGCCTATGTTGTCCTTGG
CCAGTTTCTGGTGCTAAAGAAAGATGAAGACCTNTTCCGGGNAATGGCTGAAAGACACTTGTGGC
CGCCAACGCCAAGCAGTTCCCGGGGACTGCTTCGGATGCCCTTTCGTAGAGTGGTGCCGACGCCTT
CTTGTGATGCTCTCTGGGGAAAGCTCTCAATCCCCCAAGCCCCTCATTCCAGGAGTTTGCAGCCCG
AGTAGGGGACTCCCTCCCCTTGTCCTCTTACCGNAAGGGAAAAAGGATTTGCTATTGNTCGTTACC
CTNNGGCCCGCTCNTAGAAACTAAGTNGGAATNCCCCCCGGGGCCTGCAAGGGAAATTTCNATTA
TTCAAAGCCTTTATTCGGATACCCGTCCGACCCTTCGAANGGGGGGGGGCCCGGGTACCCCCAANC
TTTTTTGGTTTCCCTTTTAAGTGGA

| | |
|---|---|
| Sequence 640 | cMhvSG078e11a1 |

AGGACGCGGGGAGGAAGTGTCGGCGCCGCCACTGTNCGGCCACAGCCTAACGCTCTTCGCTGTCG
TTTGTGGTCTCGCGCAGGGCGGCCCCGGTTCTGGTGTTTGGCGTCGGAATTAAACAACCACCATGT
CGAGCAAAAAGGCAAAGACCAAGACCACCAAGAAGCGCCCTCAGCGTGCAACATCCAATGTGTTT
GCCATGTTNGACCAGTCACAGATTCAGGAAGTTCAAAGAGGCCTTCAATATGATTGATCAGGAAC
AGAAGATGGCTTCATCGACAAGGGAAAGATTTGCATGGATATGCCTTGCTTTCTCTAGGGGGAAA
GAATCCCACTGGATGCATACCTTTGGATGCCATGATGAATGAAGGCCCCAGGGGCCCATCAATNTT
CACCATGGTTCCTGGACCATGTTTTGGGTNGAGGAAAGTTAAATGGGCCACCAAGATTCCTGGAA
GAATGGTCATTCANGAAAACCGCCCTTTTGCTTTGCTTTTNGATTGAAAAGAAAAGCCTAACCAGG
GGCACCCATTTCAAGGGAAGGATTTACCCTTANATTAAGAAGCCTNGCTTGGACCAACCCATTGGG
GGGGGGAATCCGGGNTTNTACCAAGAATTGNAGGGAAAANTGGGATTGGAGNCTGGTTACCCTTG
CCCCGGGGCCGGGNCCCGNNTCTTANNAACCTTAAGNGGGNATCCCCCCCGGGNCTTTGCAAGGG
AAATTCCGATTATTCAAAGGCCTNTATTCNGATTACCCGNCNGACCCTTCGAAGGGGGGGGG

| | |
|---|---|
| Sequence 641 | cMhvSG038c11a1 |

AGGTACTTTGGCCTCTCTGGGATAGAAGTTATTCAGCAGGCACACAACAGAGGCAGTTCCAGATTT
CAACTGCTCATCAGATGGCGGGAAGATGAAGACAGATGGTGCAGCCACAGTTCGTTTGATCTCCA
CCTTGGTCCCTCCGCCGAAAGTGAGCAGTGAGCTACCATACTGCT

| | |
|---|---|
| Sequence 642 | cMhvSG038c11a1 |

GTCTGGGNATGCCAGTGGCCCTGCTGGATGCACCATAAGATGAGGGAGCCCTGGGNAGCCTGGCC
CAGGGTTTCTGCTGGGTACCCTGCCCGGGCCGGCCCGCTCTAGA

| | |
|---|---|
| Sequence 643 | cMhvSG038c11a1 |

GAATTCGATATCAAAGCTTATCGATACCCGTTCGACCTCNAGGGGGGGGGGCCCCGGTACCCAAG
CTTTTTNGTTCCCTTTAAGTGAGGGGTTAATTGCGCCGCCTTGGCCGTAATCAATGGGTCATAGCTT
GTTTCCTGTGTNGAAATTGNTTATCCGCTCACAATTCCCACCACAACATACCGAGCCCGGGGAGCA
TAAAAGTGTAAAGCCCTGGGGGTGCCTAATGAAGTGGAGCTTAACTCACATTAATTTGCGNTGCN
GCTCACTTGCCCGCTTTTCCAGTCGGGGAAAACCTGTTCGTGCCCAGCCTGGCATTAATGAATCGG
GCCCAACCCCC

| | |
|---|---|
| Sequence 644 | cMhvSG028a02a1 |

NCCGGGCAGGTACTTTGGCCTCTCTGGGATAGAAGTTATTCAGCAGGCACACAACAGAGGCAGTT
CCAGATTTCAACTGCTCATCAGATGGCGGGAAGATGAAGACAGATGGTGCAGCCACAGTTCGTTT
GATTTCCACCTTGGTCCCTTGGCCGAACGTCCGTAGAGTTCTATAGTATTGTT

| | |
|---|---|
| Sequence 645 | cMhvSG028a02a1 |

TCGGTCAGGGACCCCGGGATGCCCGGGTAGAAGCCCAGTAAAATGAAGCAGTTTTAGGAGGCTGT
TCCTGGTTNTCTGCTGGGTACCTTCGGCCGCTCTAGAACTAAGTGGATCCCCCGGGGCTGGCAAGG
GAAATTCGATNTTCAAAGCCTTATCGGATACCCGTTNNANCCTTCGAGGGGGGG

| | |
|---|---|
| Sequence 646 | cMhvSG029c11a1 |

CCGGGCAGGTACCAGGCTAAGTAGTTGCTGCTATCACTCTGACTGGCCCTGCAGGAGAGGGTGGN
TCTTTCCCCTGGAGACAAAGACAGGGTGCCTGGAGACTGCGTCAACACAATTTCTCCGATGGTATC
TGGGAGCCAGAGTAGCAGGAGGAAGAGAAGCTGCGCTGGGGTTTCCATGGTTCCCTCTGGGTCCT
AACTGAGCAGCTCTTCTCTCCCGCGTACCTCGGCNCGCTCTANAACTAGT

| | |
|---|---|
| Sequence 647 | cMhvSG029c11a1 |

TAANTGCCGCGCTTTGGGCGTTAATCATGGNCATTAGCTGTTTTCCTGTGGTGAAAATTGGNTATTC
CGNTTCACAATTTCCACACAAACATTACCGAAGCCGGGGGAGCCATAAAAGGTTGTAAAAAGCCC
TGGGGGGTGGCCCTAAATGGAAGGTGGAAGCCTTAAACTTCNACCATTTAAATTGGCCGTTTGCGG
CCTCACNTGGCCCCCGCCTTTTTCCAAGNTTCCTGGGAAAAACCTTNTTCGGTGCCCCAGCCTTGCA
TTTTAAAATG

| | |
|---|---|
| Sequence 648 | cMhvSG038f08a1 |

AGGTACTTGTTGTTGCTTTGTTTGGAGGGTGTGGTGGTCTCCACTCCCGCCTTGACNGNAGCTGNTA
TCTGCCTTCCAGGCCACTGTCACGGCTCCCGGGTAGAAGTCACTTATGAGACACACCAGTGTGGCC
TTGTTGGCTTGAAGCTCCTCAGAGGAGGGCGGGAACAGAGTGACCGAGGGGGCAGCCTTGGGCTG
ACCTAGGACGGTCAGCTTGGTCCCTCCGCCGAACACTATGGCACTGAGGCTGTAAGTCCCATGTTG
AACAGTAATTAATCAGCCTCGTCCTCAGGGCTGGAGGCCCCGAAATAAGTCAGGGGAGGCTGTGG
GTCCCANACTTTTTGAGCCANGAGGAAGCGGGTCAGGGGATCCCTGAGGGGCAAGAGAATTTTCC
AAACATCACAGTTTTGGGGAGCCGCCCGTGAGGAAAATCNTGTTGGTACCNTGCCCCGGGCCGGC
CCGCTCTANGAACTAAGTGGGATCCCCCGGGCCTTGCAGGGAATTTCNGATATCAAGCTTTATCGG
ATTACCCGTTCGACCCTCNAAGGGGGGGGCCCCGGTTACCCCAAGCTTTTGGTTNCCCTTTTAAGT
GGAGGGGT

| | |
|---|---|
| Sequence 649 | cMhvSG025h08a1 |

AGGTACAACAAGCGGGAAACGATAGAGGCTTGGACTCAACAAGTCGCCACTGAGAATCCAGCCCT
CATCTCTCGCAGTGTTATCGGAACCACATTTGAGGGACGCGCTATTTACCTCCTGAAGGTTGGCAA
AGCTGGACAAAATAAGCCTGCCATTTTCATGGACTGTGGTTTCCATGCCAGAGAGTGGATTTCTCC
TGCATTCTGCCAGTGGTTTGTAAGAGAGGCTGTTCGTACCTGCCCG

| | |
|---|---|
| Sequence 650 | cMhvSG025h08a1 |

GATCCCCCGGGCTTGCAGGGAATTCGATTATCAAGCTTTATCGATACCGTCCGACCCTCGAGGGGG
GGCCCCGGTACCCAGCTTTTTGTTCCCTTTTAGTTGAGGGGTTAAATTGCCGCGCTTGGGCGTTAAT
CATGGGTCATAAGCTGTTTTCCCTGTGTGGAAAATTTGTTTATCCCGCTCACAAATTTCCCACCACA
ACAATAACGAGCCCGGGGAGCCATTAAAAAGTTGGTAAAAAGCCCTGGGGGGTGGCCCTTAAATG
AAGTGGAGGCCTAAACTTCCACAATTAAATTTGCCGTTTGGCCGCCTNCAACTTGGCCCCCGGCTT
TTTTCCCAAGTANCGGGGGAAAANCCCCTTGGTTCCGTTGGCCCAAGGCCTTGGCAATTTAAAATT
GGAAAA

| | |
|---|---|
| Sequence 651 | cMhvSG045d02a1 |

ACGCGGGAAATATATTATATATGGATGTGTGTGTGTGCGTGCGCGTGAGTGTGTGAGCGCTTCNGC
AGCCTCGGCCTAGGTCACGTTGGCCCTCAAAGCGAGCCGTTGAATTGGAAACTGCTTCTAGAAACT
CTGGCTCAGCCTGTCTCGGGCTGACCCTTTTCTGATCGTCTCGGCCCCTCTGATTGTTCCCGATGGT
CTCTCTCCCTCTGTCTTTTCTCCTCCGCCTGTGTCCATCTTGACCGTTTTTCACTTGTCTCCCTTTTCT
GGACCTGTCCCTGCCAATGGCTCCAGCTTGTCGTNCTGACTCTTGGGGTTNCGTTTGGGGGGACAT
GGAAGAATTTTTTATTTTTTTTGGTGGAAGTTGAAGACTGGAAGGGGATCGGTAGGAATTTTTTTT
ACAAAATTNTGTGAANTANTTTTGGAACAAAATTTCTTGGGGGTTGCCCGAAGNTGGTTGAAGAA
GGTTGGTTGNAAGCNAAGGGGGCCTTTTGGCNTTCCCTGGGCCCAAACCCAACCAAATTTTCCAAA
TTGGAAAATTNCCCCCGGAACCCCCCCCCCTTAACCCCCCAATTGGCCTTGGTAACCCCTTGGCCC
CCCGGGGGCCGGGGCCCCGCCTTCTTAGGNAAACCTAAGGTNGGGAATTCCCCCCCCCGGGGCCT
TGGCAAGNGGAAAATTTCCGAANTANTCCAAANGCNTTTAATTCGAATAACCCCGGTCCNGNAAC
CCTTCNGGAGGGGGGGGG

| | |
|---|---|
| Sequence 652 | cMhvSG002h01a1 |

CCGGGCAGGTACGCGGGGCCCTCTCTGTCTTCTCTGCAGTGGGAGCAGCTCTCCTGCCACGGCTCC
TCACCCCCTGAAAATGTTCGCCTGCTCCAAGTTTGTCTCCACTCCCTCCTTGGTCAAGAGCACCTCA
CAGCTGCTGAGCCGTCCGCTATCTGCAGTGGTGCTGAAACGACCGGAGATACTGACAGATGAGAG
CCTCAGCAGCTTGGCAGTCTCATGTCCCCTTACCTCACTTGTCTCTAGCCCGCAGCTTTCAAACCAG
CGCCATTTCAAGGGACATCGACACAGCAGCCAAGTTCATTGGAGCTGGGGCTTGNCACANTTGGG
GTGGCTGGNTCTTGGGCTGGGAATTGGNACTGGTGTTTTGGAANCCCAATCAATTGGGTATGCCCA
GGAACCTTTTNTTNANCAACAGCTTTTTTTCTAACGCCAATTTTGGGCTTTTGCCCTTTCGGAAGGG
CCATGGGGGCTNTTTTTGTNTTGAAGGGGANGCCCTTTTNTNATCCTTNTTTTGNCCATTGTNGNAA
AGGAAACCCGNTTTTCAACCCTTCCCAATAAAGTTNTTCCCCCGTTTTTGGGTTGGGNCCCCCCGG
GGGGGTNCCCTTTTTCCTTANAACCCTCCCCCAAAGCCAAACCCTTNGGGGGAAACCTGGGGTTGG
GCTTNAAGGGTTTTTGGCCCCNANAAAAAAAACCAAAANAAAANTACCTGTNTTTTAANTGGGGA
AAAAAAAAAAAAAAAAAA

| | |
|---|---|
| Sequence 653 | cMhvSG070b03a1 |

AGGTACAGAACTTTACAGAATAGAGGCAATACTTTAGCTTAAGNNNGTCTGCTGACCAGAGAATG
GANTTCTGCGTGGACTCAAGGAACAAAAGGAAACTAGGCAGGGAAGGGGAAGAAAAGTGCCCAT
CTGAATCAAACTTCAGCTGCCATCAGGGCACATCTTGTGGTGGTCACAGATTGTAGGCTGTTTTTT
GGAAGATTCGGGTTCAGCACAGGATTCCATTTGTCTACTTGGCTACACCCCTGGCTGAGGTGCCCA
TGAGGTCCAATGTCACTCAAAGTTCCTGGGCCCAGCTCAAAACTCCCCGCAAGCAAAAAGAGTCC
CCAAAATTTAGTATCAAAGTTCCTCNCGGGAAGGTCATTCCCTATCAGTTGGCAAAAGCGGGTAAG
ACCGCCCCGAAAAGCCCAATCTCCCCACCGTTGTCCCGGTATTTCGGGGNAGTTTCATTTAGCCCG
AAGCCCAGCCAGGCGCCTCACCGGGGACCAGTGCCTGGAAAGCCCATAAGTGGGAAAAGCCTTTT
CCGCCATTGGGGNCCTTCGGGTGGGGAGGGACCCCCCGGCGTTACCCTTGCCCCGGGNCCGGGGC
CGCTTCTTAAGAAACTTAAGTNGGAATCCCCCCCGGGGCCTTGCAAGGGAAATTCCGAATATTCAA
AGGCCTTTATTTCCGGATTACCCGGTNCGAACCCTTTTGAANGGGGGGGGGGCCCCCGGGTTANCC
CCAGCCTTTTTTGGTTT

| | |
|---|---|
| Sequence 654 | cMhvSG050g10a1 |

AGGTACGCGGGGATACTTTCTGAGAGTCCTGGACCTCCTGTGCAAGAACATGAAACATCTGAGGTT
CTTCCTNCTCCTGGTGGCAGCTCCCAGATGGGTCCTGTCCCAGGTGCAGCTGCAGGAGTCGGGCCC
AGGACTGGTGAAGCCTTCACAGACCCTGTCCCTGACCTGCACTGTCTCTGGTGGCTCCATCAGCAG
TGGTAGTTTCTTCTGGACCTTGGATCCGGCAGCCCGCCGGGAAAGGGACTGGNAGTGGATTGGGC
GAAATCCTTACCAGTGGGGAAGCACCGACTACAACCCCTTCCCTTCAAAGAAGTCCGAGTCTCCAT
TGTCAAGTTGGGAAGAAAGTCCCAAAGAACCAAGTTCTCCCTTGAANGTTTGAAGTTTCTCTTGAC
CCGCCCGTCANGACCGCCGGCCCGCTTCTTAGAAACTAAGTTGGGATCCCCCGGGCCTGGCAGGG
AATTCGATATTCAAGCTTANTCGAATACCCGTTCGTACCCTCGGAAGGGGGGGGGCCCGGTTACCC
CAGCTTTTTTGTTCCCTTTTAGTGGAGGGGTTAAATTGGCGCCGCCTTGGGCCGTAATCATGGGTCA
TTAAGCTGGTTTTCCTGTGGTGGAAAATTTGGTTTATCCCGCTCAACAAATTTCCCACAACAAACAT
TACCGAAGCCCGGGGAAGCCATTAAAANGTGTTAAAAAGCCCCTG

| | |
|---|---|
| Sequence 655 | cMhvSG052h11a1 |

AGGTACTGCATCTTTAATCTCTTGCTGGGCACGCCGCCCAGATTGGCCGAGGCCTCGCTCCGGACC
ATCGCAGACGCCGCCACTAGGAGAAGCAGCAGAAGCCTCATCTTAAATGAGCCAGCCACTT

| | |
|---|---|
| Sequence 656 | cMhvSG052h11a1 |

CGGTACCCAGCCTTTTGTTCCCTTTAAGTGAAGGGTTAATTGCCGCCGCTTGGCGTAAATCAATGG
TCATAAGCTGTTTCCTGGTGTGAAAAATTGTTATTCCCGCTTCACAAATTCCACACAAACCATTACC
GAGGCCCGGGGGAGCCAATAAAAGGTGGTTAAAAGCCCTTGGGGGGTGGCCNTAAATTGGAAGT
GGAAGGCCTAAACTTCACCAATTTAAAATTTGGCGGTTTTGCCGGCTTCAACTTGGNNCCCGCCTT
TTTCCCAAGNTCGGGGGAAAAAACCNTTGGTCCGGTGGCCCAAGCCTTGGCAATTTAAAATGGAA
AATTCG

| | |
|---|---|
| Sequence 657 | cMhvSG045d12a1 |

AGGTCGGCCGAGGTACGCGGGAACTCTGTCAACGAAGGCTTGAACCAACCTACGGACGACTCGTG
CTTTGACCCCTACACAGTTTCCCATTATGCCGTTGGAGATGAGTGGGAACGAATGTCTGAATCAGG
CTTTAAACTGTTGTGCCAGTGCTTAGGCTTTGGAAGTGGTCATTTCAGATGTGATTCATCTAGATGG
TGCCATGACAATGGTGTGAACTACAAGATTGGAGAGAAGTTGGGACCCGTCAGGGAGAAAATGGC
CAGATGATGAAGCTGCACATGTCTTGGGAACCGGGAAAAGGAGAAATTCAAGGTGTGACCCTCAT
GGAGGCAAACGTGTTACCGATGATNGGGGAAAGACCATTACCACGTANGAAGAACAGTTGGCAG
GAAGGGAATATCTCGGTGCCATTTGCTCCTGCACATGCTTTTGGGAGGCCAAGCCGGGGGCTTGGC
CGCTTNTGAACAAACTTGCCGCNAGAACCTGGGGGGTGAACCCCAGTCCCGAAAGGCACTACTGG
GCCAAGNCCTACCAGCCCAGTATTCTCAGNAGATTNCCATCCAGAAGAACCAAAACCCCTTAATG
GTTTNATTTGGCCCCCAAATTTGGAGGTGCCTTTTCATTGGCCCTTTTAAGAAATGTTACCCCTTGC
CCCCGGGGCCGGGGCCCNCTTCTTAAGAAACTTAGGTGGGGNATCCCCCCCCGGGGGCCTGGCAA
GGGNAATTTCCNGAATTTCCAANGCCTTTATTCCGAATTANCCGNTNCNAANCCTTCCNAANGGGG
GGGGGGGCCCCCGGGNTNNCCCCCAACTTTTTTTTGG

| | |
|---|---|
| Sequence 658 | cMhvSG040a08a1 |

AGGTACTTTAGGAGACCCAGGCGGGCAGATTGCCTGAGGTCAGGAGTTTGAGACCGGCCTGGCTA
ACATGGTGAAACCCTGTCTCTACTAAAAATACAAAAATTAGCCGGGCATGGTGGCTCACGCCTGTA
GTCCCAACTGCTTGGGAGGTTGAGGCAAGAGAATCGCTTGAACCCAGGAGGTGGGGGTTGCAGTG
AGCCGAGATCGCGCCACTGCACTCCAGCATGGGCGACAGAGCAAGACTCCATCTCAAAATAAAGA
AAGAAAGAAACAAAGAAAAGAAAAGCTTATATTGAACTTCTCTAAAAAAAGAAAAAAAAGAAAG
CCTGATGCACACAAATCTAAATTTGGCAAGTCGATCAATTAAAGGATATTTATTTGCATCACAAAA
TAATTCTTTACTCCCCCCAAAAATCAATAAAAAGTTCAAATAGCAACTTTTCCTAATGTGTTTAAA
ATGTAATCACCAAATACATGTGTCCCCAACTTTCTTTCCAGTTATAATTCTATTGGNGTAAAGGGA
NGTTACCTGGAAGTGAGGCAATAAAGAAGAGTTGAGCTTCANACCTGCCTGGAGAGAGCCGTGGT
TCTTTTTTTANAGTTTTGANGGAAATNGGTTNGGGGGCACCAAAATTNTTTTAAATCTTTTTT

| | |
|---|---|
| Sequence 659 | cMhvSG001e10 |

ACCCAGGATCTGGAAGGAAAGGGCCAAGCTGGGCTGTGGCATCCACTGGACCCTAGAGTCTTCAT
TGGGCAGGGGCCTCAGAAATCCACAAAAGACTCCCCAGTGGCTGTTCCTCTTTCCCAACGAGGCTT
GGACCCCCTTCCAGCCATTTGGGAACTCAAGCAGGAAGGAAGGTTCCTTAGGACAGGTTCCTGGC
ATGGCAGGTTCCCCTGGGAAGTGGTCGGAGGGCCCTCCCACCTTCTTGATGCCAGCAAGAAGTCA
AGGGCCTTTCCTGCTTCCCTGAGGACAACAATCAGGGCTTTCTTGCGGACTTGGGCCTTCTGGTTCA
CACTGGCAACGTTTCAGAACCCCAANGTACCCTCGGCCGGTTCTTAGAACCTAGTTGGGATCCCCC
CGGGCCTGCAAGGAATTTCCGATATTCAAGGCTTTATTCGGATACCCGTCCGAACCTCNAGGGGGG
GGGCCCCGGTTACCCCANNCTTTTTGGTTCCCTTTAAGTGGAGGGGTTTAATTTGCCGCCGCCTTGG
CCGTAAATNAATTGGGTCAATTAAGCTTGTTTTCCCTGTGGTGGAAAAATTGTTTAATTCCCGNTTC
ACAAATTTTCACAACAAACCATTACCNANNCCCGGGGAANCCATTAAAAGTGGTAAAAAGCCCTT
GGGGGTTGCCCCTAAATGGAAGTTGAAGCCTAAACTTCACAATTAAATTTGCCGTTTGNCGCCTCA
ACTTGCCCCGCTTTTTCA

| | |
|---|---|
| Sequence 660 | cMhvSG004f06 |

CCGCGGTGGCGGCCGCCCGGGCAGGTACAGAATGGCGGTCCTGCTGACTTGGCTGGGCTAGAGGA
TGAGGATGTCATCATTGAAGTGAATGGGGTGAATGTGCTANATGAACCCTATGAGAAGGTGGTGG
ATAGAATCCAGAGCAGTGGGAAGAATGTCACACTCCTAGTNTGCNNAAAGAACGCNTANNNTTAT
TNCCAANCTNNGAAAATCCCTATTGNTTCCTCCCTGGCTGATCCACTTGACACCCCTCCAGATTCTA
AAGCNATGTANTAGCGTTNTNAATCCCNNCCATNNCTNNGGNNNGGCCCAANGAACCGCGGCCCN
NCAGNTACCTTCTTGGCNCGNTCTANAACTANGTGGGGATCCCCCCGGGCCTGCAAGGGAATTTCG
ATATCAAGCTTAATCCGATACCCGTCGGACCCTCGAGGGGGGGGGGCCCGGGTACCCCAAGCTTTT
TTGTTCCCTTTAGTGGAGGGGTTTAATTTGCGCCGCTTGGCGTAAATCCATGGGNCAATAAGCTGT
TTTCCTTGTGGTGAAAAATTTGGTTNTTCCCGCTNCACCAAATTTCCCACCAACCAAACCATTACCG
ACCCCCGGNGGGANTCCANTTAAAAGGTGGTAAAAAACCCCNTNGGGGGGGTGGCCCCTNAANTG
GAAGNTGGANGCCTTAAACTTCANCATTTTAAAATTTGGCCGTTTGCCGCTTCACCTGGCCCCCGC
TTTTTCCAAGTTC

| | |
|---|---|
| Sequence 661 | cMhvSG008d05 |

TCCACCGCGGTGGCGGCCGCCCGGGCAGGTACGCGGGGAGACATACACTGGAGTGATGCAACTAC
AAACCAAGGAACACCAAGGACCACCAGCAATGACTAGAGCTAGGAGAGAGGCATGGAATAGATT
CTCCCACAGAGCTGCCAGAAGGAACCAGCATTGCCAACATCTTATTTCAGACTTCTAGCCTCCAGA
ATTGTGAGAGAATAAATTTTTGTTGTTTTCAGCCTTCCAATTTGTGATAATTTGCTATGGTAGCCCT
AGGAAAATAATACATCTGGATTCCAGCTTTCCACTCACATCATCGTTTTCTCCATCCTTCCCATGTC
TACATATTGTTGTTCCAGATTAAAGATATCTTGATGTCACAGGTGCTGGGAATTGTTTTTGTAACTC
TTTCTCTTGGTGGCTCTGTGGTGATTGACTCCCAAGGACAAAAGGANGCTTACCNNNAAAANNNN
NNNNNNNNNNNGTACCTCG

| | |
|---|---|
| Sequence 662 | cMhvSG009c03 |

AGGTACTCTCCAAGCTGCTCAAAAAGCTCACAATTTTGTTTGATTAAATTCTGAGGCTCTTCCACA
AGAGGTTTAAATTCATCGAACACTTTGGCATAGCATTCATGAGGATCTGCAGCGGCGCAGCACTTC
TCTAGAGTGGTTTCATATGTCTTGGCAAGTCTCAGCAGCAGCACGACAGAGTAATCAGGATGCCTT
CTTGCATATTCATACAAAAACATGCCCAGGAAGACATCCTTTGCCTCAGCATAGTTTTTGCAAACA
TCCTTACTTTCAACAAAATCAGCAGCCTAATGGAAGGCAAGTCAGCAGGGCATCTCATCATTTTCC
ACTTCGGCAATGCCCCTGGCGTACCTGCCCCGGGCGGCCCGCTCTAGAACTAAGTNGGGATCCCCC
CCGGGCTGCCAGGGAAATTTCGANTNTCAAAGCCTTATTCCGATTCCCGTNCGACCCTCGAGGGGG
GGGGGGCCCCCGGGTACCCCCAACTTTTTTT

| | |
|---|---|
| Sequence 663 | cMhvSG009c03 |

AGGTACTCTCCAAGCTGCTCAAAAAGCTCACAATTTTGTTTGATTAAATTCTGAGGCTCTTCCACA
AGAGGTTTAAATTCATCGAACACTTTGGCATAGCATTCATGAGGATCTGCAGCGGCGCAGCACTTC
TCTAGAGTGGTTTCATATGTCTTGGCAAGTCTCAGCAGCAGCACGACAGAGTAATCAGGATGCCTT
CTTGCATATTCATACAAAAACATGCCCAGGAAGACATCCTTTGCCTCAGCATAGTTTTTGCAAACA
TCCTTACTTTCAACAAAATCAGCAGCCTAATGGAAGGCAAGTCAGCAGGGCATCTCATCATTTTCC
ACTTCGGCAATGCCCCTGGCGTACCTGCCCCGGGCGGCCCGCTCTAGAACTAAGTNGGGATCCCCC
CCGGGCTGCCAGGGAAATTTCGANTNTCAAAGCCTTATTCCGATTCCCGTNCGACCCTCGAGGGGG
GGGGGGCCCCCGGGTACCCCCAACTTTTTTT

| | |
|---|---|
| Sequence 664 | cMhvSG015c09 |

CCGGGCAGGTACCTGGGAGTGGCCTTCTGTGCCTGCCACTGTGCTTCCCACATTGCTTAGTCACAC
ACATAACTGGGAGGTGCTGTGTTCCCAGTTTTTGTGAGTGCATTGAGCCCCTAGTGGTTCTACCCCT
TAGCAATAACTGTCCCTGGAACAGGTGTCATCACTGTAGAAATGCAGGTTACAGCCCTTGCAGAAC
ACANAGATTGGGCCCATGAATTACACCTGAGCTGCCCTNCTTTTGTTAATTGATGAGTTTGATCAA
GATCAGGAAGGTGGTGATGCAAAACCGGATGGCCTTAGACATAGTCACAGCTGCTCAAGGTGGCA
CCTGTGCCCTTGTANGGACAGAAGTGTTGTACCTTTNGCCGCTCTAAAAACTAGTNGATCCCCCGG
GGCTNGCAGGGAATTNGATAATTCAAANCTTTATTCGAATACCCGTTNNACCCNTCNGAGGGGGG
GGGGCC

| | |
|---|---|
| Sequence 665 | cMhvSG016d10 |

CGAACGCAGCCATAGCGCGGANAAGATGGCAACAGTTACCCCCGCGTACCTGCCCGGGCGGCCGT
GGCTGCCCAGACGTATTTGGCGTCGCAGTAGCCGACAATGGCGGCCTCCCGGCAGCAGCCATCGC
ACATCAGGTTATCCACGTAGCTCTGCCAACCGGCCATCTTCGAGCCCCCCCGCGTACCTCGGC

| | |
|---|---|
| Sequence 666 | cMhvSG017e10 |

ATTCATCATGGATGCTATGAGTNAGCCAGGGGGCAGGCTTGCCATGGGTTTTGTGACACCCCCATC
CAAAGCTCACCATGTTGCATCCCGCCCATTGTNTGNGGGACCCCAAGTTTCTAGCCATGTCCAGNT
CTTCACAAAAGCTGGATGCACATGCCAAGGCAAGCCATCCACAGCTGCTGCTGGAAGGGTGGTGC
AGATCTAACAGNNGGAGACATTGGCCACCTCAGCATAGGTGTGAGCCCAGNCCACAATGTTGTTG
GAGCATGCCAACCTGTGGCTG

| | |
|---|---|
| Sequence 667 | cMhvSG017f04 |

CCGGGCAGGTACGCGGGCGGGCTGAATAAAGCCGTGTCTCATCTACCTGCTGTNTCCCAAGTGTTC
TTCCAGCTCCCTGCCCCTNATCAACCNACTCTCCTCAGACCTCAGCTGGGGCTTGAACCTGATAATT
GGTGTAGTCATCAGGATGAGCTGTACCT

| | |
|---|---|
| Sequence 668 | cMhvSG025a06 |

GCGGCCGAGGTACTCTCCAAGCTGCTCAAAAAGCTAACAATTTTGTTTNGATTAAATTCTGAGGCT
CTTCCACAAGAGGTTTAAATTCATCGAACACTTTGGCATAGCATTCATGAGGATCTGCAGCGGCAC
AGCACTTCTCTAGAGTGGTTTCATATGTCTTGGCAAGTCTCAGCAGCAGCACGACAGAGTAATCAG
GATGCCTTCTTGCATATTCATACAAAAACATGCCCAGGAAGACATCCTTTGCCTCAGCATAGTTTTT
GCAAACATCCTTACTTTCAACAAAAATCAAGCAGCTAATGAAGGCAAGTCAAGCAAGGCCATTCT
CGGCATTTCCACTTCGGCCAATGCCCCGCGTACCTGCCCCGGGCCGGCCCGCTCTAGAACTAAGTG
GGATCCCCNCGGGGCTTGCAGGGAAATTCGATATTCAAGGCTTATTCGATACCCGTTCGACNCTCT
AGGGGGGGGGCCCCGGTTACCCCANCCTTTTGGGT

| | |
|---|---|
| Sequence 669 | cMhvSG025a10 |

TGGAGCTCCACCGNGGTGGCGGCCGAGGTCTCTCCAAGCTGCTCAAAAAGCTCACAATTTTGTTTG
ATTAAATTCTGAGGCTCTTCCACAAGAGGTTTAAATTCATCGAACACTTTGGCATAGCATTCATGA
GGATCTGCAGCGGCACAGCACTTCTCTAGAGTGGTTTCATATGTCTTGGCAAGTCTCAGCAGCAGC
ACGACAGAGTAATCAGGATGCCTTCTTGCATATTCATACAAAAACATGCCCAGGAAGACATCCTTT
GCCTNAGCATAGTTTTTGCAAACATCCTTACTTTCAACAAAATCANGCAGCTAATGAAGGCAAGTC
AGCCAGGCATCTCATCATTTTCCACTTCGGCAATGCAAGTGGGGATTTTTCCAACAAGAGGTTTTT
CACAGCATTCCTTTCAGTTTACTTGGAGATCGAAATCTTGGATTTTTCACAGATTATTACCTTGGGC
AAGGGTNCCGCCTATAAAGTAAGTTGGTGGGAAAATTGGTTCAACACCGANATTGGACATTTGGC
TAACCACTTTCTTCCCTTCAGGACCCTTTTATTTAAAGTTTGGGCCAGGAAACCATTATTTTCCATT
TGGNAATTTCCCCCCCCGGCGGTAACCNTTGGCCCNCGNGGGCCGGGGCCCGNCTTCTTAAGGAA
ACCTAAGGGTGGGGAATTCCCCC

| | |
|---|---|
| Sequence 670 | cMhvSG025f03 |

AGGTACAAATTGACCAGGCTGTTGACGGCTGCCTCCACGTCGGTGGAATAATTCTGACGAATCTGG
GAGCTCATGGTTGGTTGGCAAGAAGGAGCTAACCACAAAAACGGTGCCGGCAGGTCCCAGAAGCA
GGAGATGGCCGAGAAGATGGTCCCGGAGGTTGCAAGCGGAGAGGAAATCGGAGGGCGGTCGGAG
GCTGGAAGAGAGTCCCCGGATCTGTTCCGTCCAAACACTTGTTGAAGCAAGGAGACAGGACCCCG
CGGGACCGCCGAAACTTGCCCCCGCGTTACCCTGCCCGGGGCCGGCACGCTCTTAAGAAACCTAGT
GGGATCCCCCCGGGCCTGCAAGGGAATTCGATATTCAAGCTTTATTCCGATACCCGTCNGACCTTC
TGAGGGGGGGGGCCCCGGGTTACCCCAAGCCTTTTTGTTCCCTTTTTAGTGGAAGGGGTTTAAATT
TGGCGCCGCCTTTGGCGGTAAATCAATNGGGNCATTAAGC

| | |
|---|---|
| Sequence 671 | cMhvSG025f04 |

AGGTACGCGGGGGCAGTTCGGCGGTCCCGCGGGTCTGTCTCTTGCTTCAACAGTGTTTGGACGGAA
CAGATCCGGGGACTCTCTTCCAGCCTCCGACCGCCCTCCGATTTCCTCTCCGCTTGCAACCTCCGGG
ACCATCTTCTCGGCCATCTCCTGCTTCTGGGACCTGCCAGCACCGTATTTTGTGGTTAGCTCCTTCT
TGCCAACCAACCATGAGCTCCCAGATTCGTCAGGAATTATTCCACCGACGTGGAGGCAGCTCGTCA
AACAGCCTGGTCAATTTGTACCTTGCCCGGGGCGGCCGCTCTTAGAACCTAGTGGGATTCCCCCGG
GGCCTTGCAGGGAAATTCGATATTCANAGCTTAATCCGATTACCGTCGTACCCTANGNAGGGGGG
GGGGGCCCCGGTTACCCCAAGCTTTTTGGTTTCCCCTTTTANTTNGAGGGGGTTAAANTNTGGCGG
CCGCCT

| | |
|---|---|
| Sequence 672 | cMhvSG025g02 |

AGGGCGAATTGGACTCCACCGCGGTGGCGGCCGCCCGGGCAGGTACGCGGGTGCCCGACTCATCA
CAGAAACCAATTGCCAGCTGTGGGTGGTGGAGGAGCAGAGTGTTAGCCAAATCGATGGTGACTTT
GAAGACTACAAGCGGGAGGTGTTGGAGGCCCTGGGTGAAGTCATGGTCAGCCGGCCCCGAGAGTG
AAGCTTTCCTTCCCAGAAGTCTCCCGAGAGACATATTTGTGTGGCCTAGAAGTCCTCTGTGGTCTCC
CCTCCTCTGGAAGACTGCCTCTGGCCTGCAGCTTGACCTGGCAACCATTCAGGCACATGAAAGGTG
GAGTGTGGGCCTTGGATGTGGACCCGGGNATCCCACTCTTGATTGCATCCCATTTCTCTTGAAAAG
GACTTTGTTTTGTTTCTGCTTTCTTCTTNATATAAACTGGAGCCTGGGCCCTTATCCCTTTTGGGCAT
CCCCCCTTAAACAAAACAAGAGGGTGGACCACCCTTAATTGGTGAGGGTTCCNATCCCAGCCCAA
GTTTAATGTGGGCCCTATTGTTCTTCAAGGACTCTTCAATCNACTTCAAGNAANGCCCTGCCCTCTG
GATTTTAACCCCTTACAAGCTTTTCAAGGGCCCCANGCTTGGCCCCCCCCAAGATCTTTTTGGGGTN
GGGNGCCTNGTTCCTTTTTC

| | |
|---|---|
| Sequence 673 | cMhvSG025g03 |

TTGGAGCTCCACCCGCGGTGGNGGCCGCCCGGGCAGGTACTGTGAGGTTTGATTTGTGTGACAGA
ATCTGGCTTCCAGAAGTCAATCTGGGTCGTGCTGGTCAACTCGCGGATTATGTTAATGTGATTTTCA
TCTTCAACGTTAACACGGAACACCTTCTCGCCTTCAAAGTGCTCACCACCATGATGAGCAGATGCC
AGGGCCACAGTCACCAGAACCAAGAGTGCCAACATTGTGTCTGACCAGGTCTAGTGGGGTAAGGT
CTCATCTCCCGCGTACCTCGGCCGCTCTAAGAACCTAGTTGGATCCCCCGGGGNCTGCAGGGAATT
TCCGATATCAAGCCTTTATCGATTACCCGGTCGGACCTTCGGAGGGGGGGGGGCCCCGGTACCCAA
GCCTTTTTGTTTCCCTTTAAGTGGAAGGGTTTAAATTGGCGCCGCTTGGGCCGTTAATCATTGGGTC
CATTAGGCTGGTTTCCTGGTGGTGGAAAATTTGTTAATTCCGCTTCACCAAATTTTTCCACCAACCA
AACAATTACCGNAGGCCCCGGGGNAAGCCCANTTAAAAAGTGNTAAAAAGCCCCTTGGGGGGGT
GG

| | |
|---|---|
| Sequence 674 | cMhvSG032e06 |

CCGGGCAGGTACCACGATGTATAGAGCAACACTGGGGTAAGGTCACTGTGGGATGGTTGCCTGCT
GAGACCTGTGCAAACGTAACACATGCCACCATGCCAAGGATGTGGCCGGAACAAGCAGCCCTACC
AAGGCTGGGCCCCCATGGACTTTGTGCCTGCTGGGAGTTTATAGGTCTGTGGGGACATAGGATGGC
CATATCTTGCCAGCCAACTAGACTGGACATTGTACCT

| | |
|---|---|
| Sequence 675 | cMhvSG038d07 |

CCNGGCAGGTACACCTAACCAGNAACNGAAATCATTNTNTNAGNNNCCANANCACAGAATGNNCT
TGGTGAGATTGGCCNGCGGCNTTCGAGGAACTGATTGNTGCGGCAGNTNATNAGCACTTGNNTAT
TGNTCTTGACTGACTGNGTGAGCACAGAGAGTGGACCGGTGTTAAATTCCTCCTCCTCTCGCTTCT
GCAGCTTCCTCTGGGGCCATCTCACTCTTGGGCTTGNTGAGGAGGCTCATGGATGGTCACNTACGC
TCTCCGTTTCACTCCCGTTTTCCTCCGCCGTTNGCTTGCTGCCTTGAAGGGAGAAGCCCCNCNGTAC
CTCGGGCCCGCTTCTTAGAACTAGTGGAATCCCCCCGGGGCCTGCAGGGAAATTCCGATATCAAGC
CTTATTCGATACCCGTCGACCTTCGNAGGGGGGGGGNCCCGGNTACCCANGCTTTTGTTCCCCTTT
AGGTGAGGGGTTTAATTTGCCGCGCCTTGGCGTAATCATGGGTCATTAGGCCTGTTTTCCTGGTGT
GNAAATTGTTAATCCCGCTCACAAATTCCCACAACCAAACCATTAACGGANGCCCGGGGGAAGCC
ATAAAAAGTNGTTAAAAAGCCCCTNGGGGGGNTGGCCCTAAAATGAAGTNGAAGCCTTANACCTT
CAACAATTTTAAATTTGGCCGTTTNGNCGCCTTCAACTTTGGCCCGGNTTTTTCCAANTTTCCGGGG
AAAAACCCTGGTTCGTGGCCCCAGCCTTGGCATTTNAATTGGAAATTCGGNCCCAAACNCCCCCNN
GGNGGNAAAAAGGCCGGGTTTTGCCANAATTNGGGGCCGCCTTTTTTCCCC

| | |
|---|---|
| Sequence 676 | cMhvSG038g04 |

ACGCGGGGACATTTTCTCGGCCCTGCCAGCCCCCAGGAGGAAGGNGGGTCTGAATCTAACACCAT
GACNGAACTAGAGACAGCCATGGGCATGATCATAGACGTCTTTTCCCGATATTCGGGCAGCGAGG
GCAGCACGCAGACCCTGACCAAGGGGGAGCTCAAGGTGCTGATGGAGAAGGAGCTACCAGGCTTC
CTGCAGAGTGGAAAAGACAAGGATGCCGTGGATAAATTGCTCAAGGACCTGGACGCCAATGGAG
ATGCCCAGGTGGGACTTCAGTGAGTTCATCGTGTTCGTGGCTGCAATCACGTCTGCCTTGTCACAA
GTACCTTGCCCGGGCCGGCCGCTCTAGAACTAGTTGGGATCCCCCGGGCTGCAGGGAATTTNCGAT
ATCAAGCCTTATCGATACCCGTCGACCCTCGAGG

| | |
|---|---|
| Sequence 677 | cMhvSG038g04 |

CTTTAGTGAGGGTTAAATTGCGCGCTTGGCGTAAATCATGGTCATAGCTTGTTTTCCTGTTGNGAA
ATTGTTATCCCGCTTCACAAATTTCCACACAAACAATACGGAAGCCCGGGNGCCANTAAAAGTGTT
AAAAAGCCCTGGGGGGTGCCTTAAATGGAAGTNGAGCCTAACCTTCACATTTAATTTGCGGTTTGC
CGCCTNCAACTGGGCCCGCTTTTCCCANNTCCGGGGAAAACCCTTGTTCCGTNGCCCANCCTTGCC
ATTTTAANTGAATTCNGGCCNNACCCCC

| | |
|---|---|
| Sequence 678 | cMhvSG038g06 |

AGGTACGCGGGAGTGCCCCAGGAGCTATGACAAGCAAAGGAACATACTTGCCTGGAGATAGCCTT
TGCGATNTTTAAATGTCCGTGGATACAGAAATCTCTGCAGGCAAGTTGCTCCAGAGCATATTGCAG
GACAAGCCTGTAACGAATAGTTAAATTCACGGCATCTGGATTCCTAATCCTTTTCCGAAATGGCAG
GTGTGAGTGCCTGTATAAAATATTCTATGTTTACCTTCAACTTCTTGTTCTGGCTATGTGGTATCTT
GGATCCTAGCATTAAGCAATATGGGTACCTGCCCGGGCCGGCCCGCTCTAGAAACTAGTGGGATC
CCCCCGGGCCTGCAGGGAATTC

| | |
|---|---|
| Sequence 679 | cMhvSG038g06 |

CNACCCTCNAGGGGGGGGCCCGGGTACCCCAGCTTTTTTTGTTCCCTTTAAGTGAAGGGGTTTAAA
TTTGCCGCCGCTTTGGCCGTAATCATGGGNCAATTAGGCCTGGTTTTCCCTGGTGGTGGAAAATTN
GTTTATTCCCGCTCACCAAATTTCCCNCACAAACATACCGAAGCC

| | |
|---|---|
| Sequence 680 | cMhvSG039d04 |

GCTCCACCGCGGAGGCGGCCGAGGTACNCGGGGGCTGAATAAAGCCGTGTCTCATCTACCTGCTG
TCTCCCAAGTGTTCTTCCAGCTCCCTGCCCCTCATCAACCCACTCTCCTCAGACCTCAGCTGGGGCT
TGAACCTGATAATTGGTGTAGTCATCAGGATGAGATTTAGAAGTGGTGGTGCCCCTCTTGTGACAG
CATTTGGCAGTGTGCAGTTGGGCCATCAATAAATCCAAGGTCCAAGGGAACANATGAAAAAAAAA
AAAANAAAAAAGT

| | |
|---|---|
| Sequence 681 | cMhvSG039d04 |

GAATTCNATATCAAGCTTTTCTATACCGTNTACCTTCGAGGGGGGGGGC

| | |
|---|---|
| Sequence 682 | cMhvSG041h07 |

CAGGTACGCGGGATCTATGAGAAGAAGTNTGGCCAAGTCCCCATGTGTGACGCCGGTGAGCAGTG
TGCANTGAGGAAAGGGGCAAGGATCGGGAAGCTGTGTGACTGTCCCCGAGGAACCTCCTGCAATT
CCTTCCTCCTGAAGTGCTTATGAAGGGGCGTCCATTCTCCTCCATACATCCCCATCCCTCTACTTTC
CCCAGAGGACCACACCTTCCTCCCTGGAGTTTGGCTTAAGCAACAGATAAAGTTTTTATTTTCCTCT
GAAGGGAAAGGGCTCTTTTTCCTGCTGTTTCAAAAAATAAAAGAACACATTAGATGTTTACTGTGT
GAAAGAATAATGCCTTGTATGGGTGTTGATACCGTGTGTGAAGTATTCTTATTTTATTTNTCTGACA
AAACTCTTGTGTACCTNGGGCCGCTCTAGAAACTANTGGGATCCCCCCCGGGCCTTGCAAGGAAAT
TTCNAATATCAAAGCCTTATCCGATACCCCGGGNCGGACCCTTCGGAAGGGGGGGGGGCCCC

| | |
|---|---|
| Sequence 683 | cMhvSG048a02 |

ACCTGCATCAGCATTAGTAATCAACCTGTTAATCCAAGGTCTTTAGAAAAACTTGAAATTATTCCT
GCAAGCCAATTTTGTCCACGTGTTGAGATCATTGCTACAATGAAAAAGAAGGGTGAGAAGAGATG
TCTGAATCCAGAATCGAAGGCCATCAAGAATTTACTGAAAGCAGTTAGCAAGGAAAGGTCTAAAA
GATCTCCTTAAAACCAGAGGGGAGCAAAATCGATGCAAGTGCTTCCAAGGATGGGACCACACAGA
GGCTGCCTCTCCCATCACTTCCCTTACATGGAAGTATATTGTCAAGCCCATAATTGTTTCTTAAGTT
TGCAGTTACCACTAAAAGGTGACCCAATGATTGGTNACCAAATCAGCTGCTACTTACTCCTGTAGG
GAAGGGTTAAATGTTCATTCCATCCTAAGGCCTATTCAAGGTAATAACTCTTACCCTGGGCACTTA
TAATGGTTAAAGCCTTCTACTGAGGGTGCTATTGTTCCTTTAAGNGGGATGGTTCTGACCCTTGCTT
CAAATATTTNCCCTCACCTTTTCCCAATCTTTCCCAAGGGGTACCCNTGCCCCGGGGCCGGGCCCG
CTTCTTANGAAACCTAAGTGGGATTCCCCCCCGGGGCCTTGCAAAGGAATTTCNNATTATCCAAGC
CTTTATTCGGANTACCCCGTCCGACCCTTCGNGGGGGGGGGG

| | |
|---|---|
| Sequence 684 | cMhvSG048g01 |

TCCACCCGCGGTGGCGGCNCGANGTNCGCGGGGCCNGCTGGTAGTAATTCCGCTTCCTGTCCGACT
GTGGTGTCTTTGCTGAGGGTCACATTGANGCTGCAGGTCTGAATCCGGGGTGCCTTTAGGATTCAG
CACCATGGCGGAAGACATGGAGACCAAAATCAAGAACTACAAGACTGCCCCTTTTGACAGCTCGC
TTCCCCAACCAGAACCAGACTANGAAACTGCTGGCAGAACTACCTGGACTTCNCACCGCTGTCAG
GAAGGCAATTGACCCGCTAAAGGGAGGCCGAATATCTCTTGTGTGCCGAATGGGTACCCTTGCCC
GGGGCCGGCCCGCTTCCTAAGAAACCNAAGNTGGGATGCCCCCCNGGGCTTGGCANGGGAAATTT
CGGATATTCAAAGGCTTTATCGGATAACCCGTNCCGACCCTTCTGAGGGGGGGGGGGCCCCCGGT
NNCCCANCTTTTTTGGTTCCCCTTTTANTGGAANGGGGTTTAAATTNGCCGCCGCTTTGGGCCGTAA
ANTCAATTGGGTTCCATTAGCCTTGTTTTTCCCTGGTGGTGGAAAAANTTGNTTTAATTCCCGCTTT
CACC

| | |
|---|---|
| Sequence 685 | cMhvSG050a07 |

CCGGGCAGGTTCGCGGGGGACATTTTCTCGGCCCTGCCAGCCCCCAGGAGGAAGGTGGGTCTGAA
TCTAGCACCATGACGGAACTAGAGACAGCCATGGGCATGATCATAGACGTCTTTTCCCGATATTCG
GGCAGCGAGGGCAGCACGCAGACCCTGACCAAGGGGGAGCTCAAGGTGCTGATGGAGAAGGAGC
TACCAGGCTTCCTGCANGAGTGGAAAAGACAAGGGATGCCCGTGGATAAATTGCTCAAGGGACCT
GGACGCCCAATGGGAGATGCCCAGGTGGGACCTTCAGTGGAGTTCATCGTGGTTCCGTTGGCTTGC
AATCACCNTCTGGCCCTGTCACAAGTTACCTTCGGCCCGCTTCTAAGAAACTTANTGGGATCCCCC
GGGGCTTGCAANGGAAATTTCGGATATTCAAAGCCTTTATTCCGAATACCCCGTTNCGAACCTTNN
NNAGGGGGGGGGGCCCCCCGGGTTACCCCCAAGCCTTTTTT

| | |
|---|---|
| Sequence 686 | cMhvSG050a09 |

CGAGGTACGCGGGAGAGGCGACTGTCCCCACCTGAATGCTTAAATGCCTCGTTACTGGGAGGTGTT
CTCAGAAGAGCCAAATCGAAAAATGGAGGCCGGTCCTTGCGGGAGAAGTTGGACAAGATTGGGTT
GAATCTTC

| | |
|---|---|
| Sequence 687 | cMhvSG050a09 |

GTCATTAGCCTGTTTCCCTGTGTGGAAATTGTTATCCCGCTCACAATTTCCACACAAACATTACCGA
AGCCCGGGGAGCATTAAAAGTGGTAAAAGCCCTGGGGGGTGCCCTAATGAAGTGGAGCTAACTCA
CATTAAATTGGCGTTTGGCGCTCACTGCCCGCTTTTCCAAGTCCGGGGNAAACCCTTGTTCGTGCCC
AAGCNTGCATTAATGAAATCGGCCAACCGCNCCGGGGGAAGAA

| | |
|---|---|
| Sequence 688 | cMhvSG052a02 |

CCACTAATTCAAGGACTCTTACCGTGGGAGCAACTGCTGGTTCTATCACAATGAAACCGCTGGNTT
GTGTGCTCTTGGTGCGCTCCTCTGCAGTGGCACAGTTGCATAAAGGATCCTACCCTGNGATCACCA
CTGGCATCTNTGGAAGAAAACCTATGGCAAGACAAATACAAGGGAAAAAGAATGAAGAAGCAGT
ACCTGNGGCCCGCTCTTAGAACTAGNGGGGATCCCCCCGGGCCTGCAAGGGAATTCCGATATCAA
GNCTTATCGAATACCCGTNGACCTTNNGGAGGGGGGGGGCCCCG

| | |
|---|---|
| Sequence 689 | cMhvSG053a09 |

CGAGGTACTCTCCAAGCTGCTCAAAAAGCTCACAATTTTGTTTGATTAAATTCTGAGGCTCTTCCAC
AAGAGGTTTAAATTCATCGAACACTTTGGCATAGCATTCATGAGGATCTGCAGCGGCACAGCACTT
CTCTAGAGTGGTTTCATATGTCTTGGCAAGTCTCGGCAGTAGCACGACAGAGTAATCAGGATGCCT
TCTTGCATATTCATACAAAAACATGCCCAGGGAAGACATCCTTTGCCTCAGCATAGTTTTTGCAAA
CATCCTTACTTTCAACAAAATCAACAGCTTAATGGAAGGCAAGGTCAAGCAGGCCATCTCATCCAT
TTTCCACTTTCGGCAATCCCCGCCGTACCTGCCCCGGGCCGGCCCGCTCTAGGAACTAGTGGGATC
CCCCCGGGCTGCAGGGAATTCCGATATCAAAGCCTTATCGATACCCGTCGGACCTCGGAGGGGG

| | |
|---|---|
| Sequence 690 | cMhvSG053a09 |

AGTGGAGGGGTTAATTGCGCCGCCTTGGGCCGTAAATCCATGGGGCCATAAGCCTGNTTTCCCTGT
GTGGAAAATTGGGTATCCCGCTCACAAATTTCCCNCACCAACCATTACCGAAGCCCGGGAAGCCA
TTAAAAGNTGTGAAAAGCCCTGGGGGGTGGCCCTAAATGGAGTGGAAGCCTTAAACCTNACCATT
TAATTTTGGCCGTTTGGCGGCCTCACCTTGCCCCCGGCTTTTTCCCAAGTTCGGGGGAAAAANCCCT
GNTCCGNTGGCCCCAGCCTGGCATTTAAATGGAAATTCGGGCCCCAACCCCCCCCCGGGGGAANA
AGGCCCGGTTTTGCCCTATTT

| | |
|---|---|
| Sequence 691 | cMhvSG053d10 |

ATTGGACTCCACCGCGGTGGCGGCCGCCCGGGCAGGTTCNCGGGACATTTTCTCGGCCCTGCCAGC
CCCCAGGAGGAAGGCGGGTCTGAATCTAGCACCATGACGGAACTAGAGACAGCCATGGGCATGAT
CATAGACGTCTTTTCCCGATATTCGGGCAGCGAGGGCAGCACGCAGACCCTGACCAAGGGGGAGC
TCAAGGTGCTGATGGAGAAGGAGCTACCAGGCTTCCTGCAGAGTGGAAAAGACAAGGATGCCGTG
GATAAATTGCTCAAGGACCTGGACGCCAATGGGAGATGCCCAGGTGGACTTCAGTGAGTTTCATC
GTGTTCGTGGCCTGCAATTCACCGTCTNGCCTGTCACAAGGTACCTTCGGCCGCTCTAAGAACTAG
TGGGATCCCCCGGGGCTGCAGGGAATTCCGATATCAAGCTTATCCGATACCCGTCGACCTCGAGGG
GGGGGGCCCCGGTACCCCAAGCTTTTGTTCCCTTTAAGTGAGGGGTTAAATTTGCCGCGCTTGGCG
TAATCATGGGTCAATAAGCTGTTTTCCTGTGTGAAAATTGTTTATCCCGCTTCACAAATTCCACACC
AACCATTACCGAGCCCGGGAGCATAAAAGTGTAAAAGCCTGGGGTGCCCTAAATGAAGNGGAGCC
TAACCTCACATTTAATTGCCGTTTGCGCTCACTTGCCCCGCTTTTCCAAGTNCGGGGAAAAACCCTG
GTCCGNGCCCAGCTTGCATTTAAATGGAAATTCNGGCCCAACCCCCCCCGGGGGAAGAAGGCCCG
TTTTTGCCNTTNTTTTGGGGCCGCCTTT

| | |
|---|---|
| Sequence 692 | cMhvSG053h06 |

CCGGGCAGGTACTTGCAATGGGGCCACCATGTTTTCTCCCATTAGCCAGCCCCATTCATCATGGAT
GCTATGAGTCAGCCAGGGGGCAGGCTTGCCATGGGTTTTGTGACACCCCCATCCAAAGCTCACCAT
GTTGCATCCCGCCCATTGTCTGTGGGACCCCAAGTTTCTAGCCATGTCCAGTNCTTCACAAAAGCT
GGATGCACATGCCAAGGCAAGCCATCCACAGCTGCTGCTGGAAGGGTGGTGCAAGATCTAACAGT
TGGAGGACANTTGGGCCACCTCANGCATAGGNGTGGAGCCCAAGTCCACCAATGGTTTGTTTGGA
AGCATTGCCAAACCCTGTGGGCTTGAGCCAAAAATAACTCCCCAAGNAATTNTGGNCAANACAAT
TCCCGGCCCCTTGGACCTTTGGNATTTAATTTGATGGCCCCAACTTGCACACTGGCCCAAANGANN
TNCTCACTAAGAGCGNGGCCACCAACCCAACTTNTATAAAAANGCTCATTCCCTCGATGGAACTA
ACACCCAAANTTTATCNAGGGTTTTCAAAGCCCCCCAGCTTGGAAGGGTCCTGGAGGGAAAAAGT
TGGGGTTTTGAATGGAATGGGGGCCNANGGGNAAGCCTTGGGAAAGGAAANCAACTTGNGGGGA
NGACNANGCCANGGTTNGGANGAAGAACAACGGGCNTTTTATTTCAANCCCCCCCGCCNTNNCCC
TTANGGGGCCGGTNTNTTAANAAACCTNANNGGGGATCCCCCCCGGGGNCCTTGGNAAGGNAANN
TTANATANTCCANGNCTTAAANGGANTNCCCGGGNATAAACCTTNTAANGGGGGGG

| | |
|---|---|
| Sequence 693 | cMhvSG055f03 |

CACCGCGGTGGCGGCCGAGGTACGCGGGCTGGGCAAGGCAGACTTCTCTGGAATGTCCCAGACAG
ACCCGTCTCTGTCCAAGGTCGTGCACAAGTCTTTTGTGGAGGTCAATGAGGAAGGCACGGAGGCT
GCAGCCGCCACAGCTGCCATCATGATGATGCGGTGTGCCAGATTCGTCCCCCGCTTCTGCGCCGAC
CACCCCTTCCTTTTCTTCATCCAGCACAGCAAGAACCAACGGGATTCTCTTCTGCGGCA

| | |
|---|---|
| Sequence 694 | cMhvSG055f03 |

CTGCAGGGAATTCCGATTATTCAAGCCTTATCCGATACCCGTCGACCCTACGAGGGGGGGGGCCCC
GGTACCCCAGCTTTTGTTCCCCTTTTAGTTGAGGGGTTTAAATNTGCNCCGCCTTGGCCGTAATCAA
TGGGNNCATAGCTGGTTTTCCTGTGTTGAAAAATTTGTTTATCCCGCTCACCAATTCCACANCAAA
CATACGAAGCCCGGGGTAGCCATAAAAGTTGTTAAAGCCCTGGGGGTGCCTTAAATGAAGTTGAA
GCCTAAACTCNACATTTAAATTTGGCNGTTTTGGCGCTTCAANTTGNNCCCGCTTTTTCCCAGTTCC
GGG

| | |
|---|---|
| Sequence 695 | cMhvSG058f07 |

NCNNGCCAGGTACGCGGNGGAAANGGGAGTGANNNAAGAGCNTAGTGANCATCATGAGCCTTCT
NNACAAGCCNAACANTGATATGACCCCAGNGGAGCNGCNCANGCGAGAGGAGGGGGAATTTANC
ACCGGTCCACTCTTTNTGCTCACACAGTNANTCAAGAACAATACCCAAGTGCTTATCAACTGCCGC
AACAATAAGAAACTCCTGGGCCGCTGAAGGCCTTCTATAGGCACTGNAACATGGGNGCTGGAGAA
CGTCTAAGGAGATGTGGACTGANGTACCTTTGCCGGCCGGGCAGGTACCAGAATATAGGTTCCCA
AATAGATCCCTGGTTTGTCTTTAGAGACACTGAAGGGGACAACAATAGCCAATTCGGGATTTCAAA
CACCCCACAAACTATACCTTAGGCTCTGTGAGGGCAAAAGACACAGTTTATTTCAACAACGATCTT
GTTCAACAGAACCTGGTCACCAAGTGGATNGATGGATGGGGCCAGACCCANATTGGGACAAGAAC
TACTTCAAGTGGGGTGGGCTACATTGTGCTTTGCCTTGCCCCCGGGAACACCATTGNACTTCACNT
TTTTGCAATTGCTTACATTANAACTANAAGTTTTGGCTTNCATTCAATTGAAAAATANNATAAGTT
NTNGGCANTTGAAAAACCTTAACAAAAAACCCTTTTTACCCCCGGCGTTTNCCTTTGGGGCCGGTT
TTANAAACTANTTGGATTTCCCCCCGGGCTTTGCANGGAAATTTCGATTATNCAAGCCTTTNTTTGA
NTACCCGNCCAANCCTNCNAAGGGGGGGGG

| | |
|---|---|
| Sequence 696 | cMhvSG064b12 |

TATAGGGCGAATTGGAGCTCCCGCGGTGCGGCGCCGGGCAGGTCATAATCGTTTTGTGGAGTCGC
ACAGTTCAGGTTATGGAGGCCCGTAATTACCAAAGTGTAAAAAAGGGCAAAGGAAACACNCCTNC
ATTGTAGAATAAGGCATTCAAATGTGCTGTTACCGTTTAAAGGCAGCTAATGNCAAAACAGGCAA
GTCAAGAAAAGTGGTCTGGTTTTGGAGGTGATTTTGCATCTAGAAGCATTCTCTTCTCGTGCCTCA
AAGNCTGACCACTGTAGAGCATGTCTTCTTCCTCAAGGCCAATGATACTTCAGATCCCAGATGGTT
TCATTTTTCAATTGCGGTCCAAAGAGAGGGTTGAGTTGGGCCAGAATTGCAATCAGCCAAAAGAG
ATAGCAGCAACCTGACCAGGTCACCACCATGGTAATGTAACTCCCCGGTAGGACCCTTANGGATG
AACCAAGGCCCAAGAAGCC

| | |
|---|---|
| Sequence 697 | cMhvSG064f04 |

CTTTNGGCGATTGGNNCTCCCCGCGGTGGCGGCCGAGGNANAATAGACAGCGCAGCAAANAGAA
GGCGCGGGCTGGGTGGGAAGAGGATTCGGACTCGTCACACTGCAGAGCAGCAGAGCGAGAAAGG
ATGAGAAGAGGCAGAGAAGGCGACGGCAGAAAGAAAAAGGAAAACTGCGGCCGAGGACTTNNTT
TTTTTTTTTTTTTTTTTTTTTTT

| | |
|---|---|
| Sequence 698 | cMhvSG067g04 |

GCAACACTGGGGTAAGGTCACTGTGGGATGGTTGCCTGCTGAGACCTGTGCAAACGTAACACATG
CCACCATGCCAAGGATGTGGCGGAACAAGCAGCCCTACCAAGGCTGGGCCCCCATGGACTTTGTG
CCTGCTGGGAGTTTATAGGTCTGTGGGGACATAGGATGGCCATATCTGCCAGCCAACTAGACTGGA
CATTGT

| | |
|---|---|
| Sequence 699 | cMhvSG070b06 |

CCGGGCAGGTACGCGGGACCTGGTCAGACACAATGTTGGCACTCTAGGGGGATGGTGACTGNGGC
CCTGGCNTNTGCTCATCATGGTGGTGAGCACTTTGAAGGCGAGAAGGTGTTCCGTTGTTAACGTTG
AAGATGAAAATCACATTAACATAATCCGCGAGTTGGCCAGCACGACCCANATTGACTTCTGGAAG
CCAGATTCTGTCNCACAAATCAAACCTCACAGTACCTCGGCCGCTCTAGGAACTAGTGGATCCCCC
GGGNCTGCAGGAAATTCGATATCAAAGCTTTATCGGATACCCGTCNGACCTTCGAGGGGGGGGCC
CNGGTACCCCAGCTTTTTGTTCCCCTTTAAGTGGAGGGTTAAATTGGCGCGGCCTTGGGCGTTAAT
CCANTGGTTCAATAAGCTNNTTTTCCTGGGGTNGAAAATTTGNTTATTCCCCGCTTCAACAAATTTC
CCAACACCANACAATAACCGNAGTCCCGGGGGGAGGCCATTACAAGTTGGTTAAAAAGCCCCTTG
GGCGGTNGCCCTTNAATGGAAGGTGGAAGCCTTAANCTTTCACCATTTAAATTT

| | |
|---|---|
| Sequence 700 | cMhvSG070c06 |

TATAGGGCGAATTGGACTCCACCGCGGTGGCGGCCGAGGTACCCAGGATCTGGAAGGAAAGGGCC
AAGCTGGGCTGTGGCATNNACTGGACCCTAGAGTCTCATTGGGCANGGCCTCAGAATCCACAAAG
ACTCCCCAGTGCTGTTCCTCTTCCAACGAGGCTGGACCCCTTCCAGCCATCTGGGAACTCAAGCAG
GAAGGAAGGTTCCTTAGGACAGGTTCCTGGCATGGCAGGTTCCCCTGGAAGTGGTCGGAGGGCCC
TCCCACCTCTTGATGCCAGCAGAAGTCAGGCCTTCCCTGCTCCCTGAGGACACATCAGGGCNTTCT
TGCGGGACTTGGTCTTCTGGTTCACACTTGGCACGTTCCAAGACCCAGGTACCTTGCCCGGGCGGC
CCGCTTCTAGAAACTAAGTGGGGATCCCCCCCGGGCCTGCCAGGGAATTTCGATATCAAAAGCTTA
TCTGAATACCGTCCGACCTTCGAGGGGGGGGGCCCCGGGTACCCANCTTTTTGTTCCCTTTTAGTG
GAGGGTTAAATTGGCGCCGCCTTTGGCCGTAAATCATTGGGTCAATAAGCCTGNTTTTCCTTGTGTT
GAAAAATTGGTTTATTCCGCTTCAACAAATTCCCACACAAACAATTAACGAAGCCCCGGGGANGC
CATTNAAAAGATGGTAAAAGGCCCTGGGGGGGTNGCCCCTAAATGGAGGTGGAAGCNTTAACCTT
ACCATTTAAATTTGCGNTTTTGCGGNCTTNACTTGNCCCCGTTTTTTCCAAGGCCCGGGGAAAAAC
CCTTGTTCNTTGCCCCAGCCTNGCATTTAATTGAAATNGGGCCCAACTCCCCCCGGGGGAAAAAAG
GC

| | |
|---|---|
| Sequence 701 | cMhvSG070h03 |

ATTGGACTCCACCGCGGTGGCGGCCGAGGTACAGTTTTCTCAGAAGACTCAAGATTTCGCCCACAT
CCCTTNGAGNNCCCGCTAGATCTGCCGCCCGGNTNCATTTGTCCCACTCTTCAGGACAGAGTTAGC
TGCCCTCTTTCTTTACTTCATAGTCTTTGTAAGGGCTCGGCCAAGCGTGGGCCCGTGGGATGGAGA
ATTCCTTTTGGGGAGGCTGGTTCTGCAGCTGAAAATGTGTGGAATAGGGGGCATAGAGCGTGTCCC
CTGTCTCTTCAAAACCTTGAGGTGATTTCCTCTTGAGGGGTAGGCTCTGTTCTCCACACCATAAGCT
CTTTCTTCACCGAAGTTGAGGTTTACAGGAAAGCCATCCCTCCAACAGGGATAAATCCCATGGGGG
GTTTCGTTGCTTTGTGAGCAAGCCANAAAACTCCGGGGGACCTAACANTAAAACCAACCAAGGGA
ACACCNCAGCCAATTGGGCCAGCCAANGGCGGGAGCTTGAAGGGATGGTGGTCATTCCCACCCTG
CCGGTCAAAAGGTTCAAGGGAAACATTGANGCAGGGGTNGATCCCAGGGCCCACCCCAGAATGG
GCAATGGGAAGAAGGGAAGCATCCGTTGAAGGGTAAAAATGNTGGGGGCCC

| | |
|---|---|
| Sequence 702 | cMhvSG070h10 |

CCGGGCAGGTACGCGGGGAGTCCCCACCTCTCTCAGCTTCCGGCTGGTAGTAGTTCCGCTTCCTGT
CCGACTGTGGTGTCTTTGCTGAGGGTCACATTGAGCTGCAGGTTGAATCCGGGGTGCCTTTAGGAT
TCAGCACCATGGCGGAAGACATGGAGACCAAAATCAAGAACTACAAGACCGCCCCTTTTGACAGC
CGCTTCCCCAACCAGAACCAGACTAGAAACTGCTGGCAAGAACTACCTGGACTTCCACCGCTTGTC
AGAAGGCAATGACCCGCTAAAGGAGGCCGATATCTCTGTGTTGCGGAATGGTACCCTCGGCCGGN
TCTANAACTAGTGGATCCCCCGGGGCCTGCAGGAAATTCGATATCAAGCCTTATTCGATACCCGTT
CGACCTTCGNAGGGGGGGGGGCCCCGGTACCCCAGCCTTTTTGTTCCCTTTTAATGAGGGGTTAAA
ATTTGCCGCCGCCTTGGGGCGTAAATTCATGGGTCAATTAGCCTGTTTTTCCTTGNNGTGGAAAAA
TTTGTTTATTCCCGGCTTNAACAAATTTNCCACCACAAACCATTACCGNAGCCCGGGGNAGGCCAN
TAAAAAGGTGGTTAAAAAGGCCCTTNGGGGGGTGGCCCCTNAAATGGAAGNTGGAAGGCCTAAA
CCTTCAACAATTTAAAATTNGCCGGTTTGGNCGNCTTCACTTGGCCCCGCTTTTTCCAANTTCGGGG
GAAAACCCTTGNTCCGTTGGCCNNGCTTGCAATTTAAATTGAAAATCCGGCCCAACCCCCCCCGGG
GGAGGAAGGGCCCGGT

| | |
|---|---|
| Sequence 703 | cMhvSG072a01 |

AGGTACGGAGCAATCGAGGAGGCATAACCACACTTGGGGTGGCTATAGGGCTGGAAAACGCTGA
AGATGACTGCTTTCACTGAGGTTAAGGATTGTAATATTGCCAGCTTTGTAAAGTCATTAAAGCAGA
AGTTTCTTCAGTGATCTTCTCTCTAAGAAACACCATCACCTCCATGTGCCTTACAGAGGCCCCCCCG
CGTACCTGCCCGGGCGGCCGNTCTAGAACTAGTTGGATCCCCCGGGCTGCAGGTAATTCGGATATC
AAGCTTATCCGAATACCCGTCGACCTCTGAGGGGGGGGGCCCCGGTTCCCAAGCTTTTTNGTTTCC
CTTTTAGTNGAGGGGGTTAAATTTGCCGCGCTTTGGCGTTAANTCATTGGGGTNCAATANGCTTGG
TTT

| | |
|---|---|
| Sequence 704 | cMhvSG072a04 |

ACAAGGTGCTAAAACAGGTTCACCCCGATACTGGCATCTCATCCAAGGCCATGGGCATCATGAATT
CCTTCGTTAACGACATCTTCGAACGCATCGCAGGCGAGGCTTCCCGTCTGGCCCACTACAACAAGC
GCTCGACCATTACCTCCAGGGAGATCCAGACCGCCGTGCGTCTGCTGCTTCCCGGAGAGCTGGCCA
AGCACGCAGTGTCCGAAGGTACCTCGGCCGCTTCTAGAACTAGTGGGATCCCCCGGGCTGCAGGG
AATTCGATATCAAGCTTAATCGATACCCGTCGACCTTCGAGGGGGGGGGCCCCGGTACCCAAGCTT
TTGGTTCCCCTTTTAAGTNGAAGGGGTTAAATTGCGCCGCTTTGGGCGGNAAATTNATTGGGTCAA
TAAGCTNGTTTTTCCCTGGNGGGTGGAAAATNTGGNTTATTCCCGGCTTCAACCAAATTTTTCCCA
NCAACCAAAACAATTACCGGAANGCNCNGGGGAGGCCAATAAAAAAGGTTNGTTAAAAGGCCCT
TGGGGG

| | |
|---|---|
| Sequence 705 | cMhvSG072h03 |

TTGGAGCTCCACCGCGGTGGCCGGCCGAGGTACGCGGGCATGCTGGAGATGGACAACTCAATGAA
AATTTAAAGGGAAAACCCTCAGGCCTGAGGTGTGTGCCACTCAGAGACTTCACCTAACTAGAGAC
AGTCAAACTGCAAACCATGGTGAGAAATTGACGACTTCACACTATGGACAGCTTTTCCCAAGATGT
CAAAACAAGACTCCTCATCATGATAAGGCTCTTACCCCCTTTTAATTTGTCCTTGCTTATGCCTGCC
TCTTTCCGCTTGGCAGGGATGATGCTGTCATTAGTATTTCACCAAGNAAGTAGCCTTTCANGAGGG
GTAACCTTAACAGGAGTGTCANGATCTATCCTTGTCAATCCCAAACCGTTTTTACATTAAAAATAA
GAGGATCCTTTTAAGTGCACCCCAGTGGACCTGACATTAAGCAGGCATCTTTAAACACAGCCCGTG
TGTTTCAAAATGGTACCCTGCCCGGGGNCGGGCCGCTCTAAGAACTAGTGGGATCCCCCGGGGCCT
GGCAGGGAATTCCGATATTCAAAAGCTTATCGATACCCGNTCGACCCTNGAGGGGGGGGGGCCCG
GGTNCCCAGCTTTTTGGTTCCCCT

| | |
|---|---|
| Sequence 706 | cMhvSG073a09 |

GCTCCACCGCGGTGGCGGCCGCCCGGGCAGGTNCTCCTTGAATACCACTTAGAGTCAGAAAGATA
AGGCAGCAAATCAGAATGGCAGTTTGATTCATGGTGCTGAGACTGGAGGTTCCTCTGCTGTAGGCT
CAGAATATGTCTAAGCAATTGAGGAATGTCTCCCCCGCGTACCT

| | |
|---|---|
| Sequence 707 | cMhvSG073a09 |

TAAGTTGAAGGGGTTAAATTNGCGCCGCCTTGGGCGTAAATCATGGGTCATTAGNCTNGTTTCCCT
GTGTGGAAATTGTTTATCCCGCTCAACCAATTTCCACCNCAAACCATTANCNGAANCCCCGGGGAA
GCCAATAAAAAGTTGTTAAAAGGCCCTTGNGGGGNTTGCCCCTAAAATTGGAAGGTGGAGCCTTA
AACCTTNAACAATTTANAATTTTGGCGGTTTTGGCGGCCNTCCACNTTGGCCCCCGCTTTTTTCCAA
NGTCCGG

| | |
|---|---|
| Sequence 708 | cMhvSG074a12 |

AGGTACCACGATGTATAGAGCAACACTGGGGTAAGGTCACTGTGGGATGGTTGCCTGCTGAGACC
TGTGCAAACGTAACACATGCCACCATGCCAAGGATGTGGCGGAACAAGCAGCCCTACCAAGGCTG
GGCCCCCATGGACTTTGTGCCTGCTGGGAGTTTATAGGTCTGTGGGGACATAGGATGGCCATATCT
GCCAGCCAACTAGACTGGACATTGT

| | |
|---|---|
| Sequence 709 | cMhvSG074e03 |

TTAGCTCCACCGCGGTGGCGGTCGCCNNGGGCANGTACCTACNGNGTGGCGCTGGGGTNTGGCTC
CATGACCATANATCTATTGGGGGACGTCAGAGAAACGGCGTCATGCCCAGCCACTTCAGCCGAGG
CTCCAAGAGTGTGGCCCGCCGGGTCCTNCAAGCCCTGNAGGGGCTGAAAATGGTGGAAAAGGACC
AAGATGGCGGCCGCTCTANAACTAGGNGGATCCCCCCGGGCTGCCAGGAATTCGATATCAAAGCT
TATCGATACCCGTTCGACCTCTGAGGGGGGGGGCCCCGGTACCCCANNCTTTTTTGTTCCCTTNTA
AATTGAGAGGTTAAATTTGCNGCCGCTTTGGNCGTTAAATCAATGGGTCCATAAGCCTTGNTTTCC
CTTGGTGTTGGAAAAATTTGTTTTAATTCCCGCTTCACCAAAATTTTCCCAACNACCAAACCAATTT
ACCNGAAGGCNCCGGGGGAAG

| | |
|---|---|
| Sequence 710 | cMhvSG001f04 |

CCGGGCAGGTACGCGGGGAGAGAGGTTGAGAACAACCCAGAAACCTTCACCTCTCATGCTGAAGC
TCACACCCTTGCCCTCCAAGATGAAGGTTTCTGCAGCGCTTCTGTGCCTGCTGCTCATGGTAGCCAC
TTTCAGCCCTCAGGGACTTGCTCAGCCAGATTCAAGTTTCCATTCCAATCACCTGCTGCTTTAACGC
GATCAATAGGAAAATTCCTATCCAGAGGCTGGAGAGCTACACAAGAATCACCAACATCCAATGTC
CCAAGGAAGCTGTGATCTTCAAGACCCAACGGGGCAAGGGAGGNCTGTGCTGACCCCAAGGAGA
GATGGGTCANGGATTCCATGAAGCATCTGGACCAAAATNTTTCAAAATCTGAAGCCCATGAGCCTT
TATTACATGGGACCTGAGAGTCAAAAGCTTGGAAGAAAAGGCTTATTTTATTTTTCCCCAACCTCC
CCCCAAGGGGCCAGNGGGGACCATTTANTTTTANTTATTAACCATNCNCCAAAAGAGAATTATTTT
TTTAAAATTAATTTTAAAAAGCATTAAATTTTTTTTTTTTAAAAAAGGGGTTTTTAAATTATTATTTT
TAAAGNTGGNTGGANGGGTTTTNAACTNTTATTTTTNGCAAACNATTNCTAAAGGGGNAATGGTN
AAAAANGGCAAAAAATNCCNGGGGGGGAGGGGGNTTTTTGGGTTTTN

| | |
|---|---|
| Sequence 711 | cMhvSG002f11 |

CGAGGTACGCGGGGACATTTTCTCGGCCCTGCCAGCCCCCAGGAGGAAGGTGGGTCTGAATCTAG
CACCACGACGGAACTAGAGACAGCCATGGGCATGATCATAGACGTCTTTTCCCGATATTCGGGCA
GCGAGGGCAGCACGCAGACCCTGACCAAGGGGGAGCTCAAGGTGCTTATGGAGAAAGAGCTACC
AGGCTTNCTGCANAGTGGAAAANACAAGGGATGCCCGNGGGATAAAATTGCTCAAGGGACCTTGG
ACGCCAATTGGGAGAATGCCCCAAGTGGGACTTTTANTNGANGTTCATTCGTGGTTNGNGGGCTTG
CAAATTNACGTTTTGGCCTTGTTNNCNAAAGGTACCCTTGCCCCCGGGCCNGGGCCGGTTTTTAAN
AAACTAAGGTGGGAATNCCCCCCGGGGCTTTNGCAGGGAAATTTTCGANNNTTNNAAAGCCTTTA
TTNGAANTACCCCNGCCCNAACCTTTNAAGGGGGGGGGGG

| | |
|---|---|
| Sequence 712 | cMhvSG002h09 |

AGGTACGCGGGATTGAGAGCTCTGCTATGCCACTGTTGAATTTTTCCCAAGATTCCTGTCCCTAGC
CCTCACTTCAAACTCTGCTTCCTTGGACAGATTTGGCAATAGCTTTGTAAGTGATGTGGACATAATT
GCCTACAATAATGAAAACCTACAGGAATTTTTTTATTTTTCATTTTCCCCTTAGGCATATTTAGTAT
TTTTCCCCCAGGCAGATCATTCTGAGTGTGCGAGTGTGTGTGCACATGTTACAAAGGCAACTACCA
TGTTAATAAAATATTCAATTTTGNNCTANGNAAAANTATGANGAAAAGGGTANCTGCCCGGGGCG
GCCCGGTTNTAAGAAACTAGTGGATCCCCCCCGGGCTTGCAAGGGAAATTCCGAATATTTAAGNTT
AATCCGAATANCCGGGCGNACCCTTNNAGGGGGGGGGGGCCC

| | |
|---|---|
| Sequence 713 | cMhvSG003a01 |

AAATTGGAGCCTCCACCCGCGGTGGCAGGCCCGAGGTACCNTTTTTTTTTTTTTTTTTTTTTTGATTN
GCAACAGGCAANAAGTTTATCGACNCACTAATGATTAANCAAGGAAAACNCATTTTACAATTNAA
AGACAAAACCGAACCAATANGACAAAAGAATCTGATAAAGGATTACAGGAGTAGCTGCAGCTNT
NTGGCCNCANGTTTNTTAGCAGTAGCTTCANCACNCCTTTTGTTAAGGNTGTCATACATNTATACA
TNCTGGGGGACCAGNGACTCAAGCNTGCCTGCATTTTACNTCTTTGAAATTTTTACATTCNNANAA
CCAGCCGNTTNGNNNACNNAAAAGTTTGGGNNGGTACATTTANTNCCNAACACACANGGCCCTGG
GGTTCCNNCCTGCGTTTTTATTGGCGAAATTTTTTAAA

| | |
|---|---|
| Sequence 714 | cMhvSG003a08 |

GAAAGGGTATGTTAAATAGTTCAGCCAGTAGCTCACCACAGGGATTAAGGGCATCTGCCAGAATG
ACATCAAACTTTGACTCTTGTAGTTTCATCATAAGTTTCTTATTCAAAACTGCATCTTTACAGAGCT
TGTTACTGTAGTCATAATATTCCCAACACAATTCTTGTAATTGTGAAAAATATGACCAAAATGTAT
TTTTTGAAACACCATATATCCATCTATCGAGAATTTTCAGAAGAGAATCTTCCAAATCATTTTTAGT
TAAAGATGTAGGATAAACTTCTAATTTAATAGCAGATGATTTACTGGCATTGACAAGAGTAGAAG
CCGAAGATGTCAACACAGTCACCTCATGGACCCCTCTGGACAAGCTCTTCCCAGGGATTGGTCTTC
ATATTTATCCCAATGGCTGGTATTCTGGNGGGCCCCCACTTAGCACCTTTTCANCAAGCTTTCCCAG
AGCTTAAAGTTAACCAACCTGGAGCTCCCGCGGTACCTGCCCCGGGCNGGCCGCTTCTAAGAACCT
AGGNGGATCCCCCCCGGGCCTGCANGGAANTTCCGATTNTCAAAGNCTTATTCGATTNCCGTCCGA
CCCTCCGAANGGGGGGGGGNCCC

| | |
|---|---|
| Sequence 715 | cMhvSG004h03 |

GGCTCCCATCCTCCGGAATCTGCAAAATGGCTNCTTCTTNANAAATAATGGGGAGAGGGATGGCTT
TNAGGCCAGAGATCAAGGCCCTCGAGTATTAACTTGAGCATTTGGGCACAAAATAGACACTTTTG
GATTTTCCCGTCTTTTCCAACACCAAGGATGAGATTATCAAAAGATGTGTTAAATTAATTTGTACCT
CGGCCGCTCTAGAACTAGCTGGATCCCCCGGAN

| | |
|---|---|
| Sequence 716 | cMhvSG004h03 |

CGATAACCGTCGACCCTCGAGNGGNGGGGGCCCNNGNTACCCCAGCTTTTTGTTTCCCTTTTAAGT
GGAGGGGTTAAATNTGGCGCGCTTTGGGCCGTAAATCATGGGGCATAAGCCTGGTTTTCCTGTTGT
GGAAAATNTGTGTNTTCACGCTCACAANTTTCCACNCNACATACCGANCCCGGGAANCCATTAAA
NNTGTAAAAGCCTGGGGG

| | |
|---|---|
| Sequence 717 | cMhvSG005h10 |

GCGGNGGCGGCCGAGGTACTCTCCAAGCTGCTCAAAAAGCTCACAATTTTGTTTGATTAAATTCTG
AGGCTCTTCCACAAGAGGTTTAAATTCATCGAACACTTTGGCATAGCATTCATGAGGATCTGCAGC
GGCACAGCACTTCTCTAGAGTGGTTNCATATGTCTNGGCAAGTCTCAGCAGCAGCACGACAGAGT
AATCAGGATGCCTTCTTGCATATTCATACAAAAACATGCCCAGGAAGACATCCTTTGCCTCANCAT
AGTTTTTGCAAACATCCTTACTTTCAACAAAATCANCAGCTAATGAAGGCAAGTCANCAGGCATCT
CATCATTTTCCACTTCGGCAATGCAGTGGGATTTTTCCAACAGAGGTTTTTCACAGCATTCCTTCAG
TTTTACTGGAGATCGAATCTTGATTTTCACAGATATACTTGGNAAGGTCCGCCTATAAGTAAGTTG
GTGGAAATTGTTNAACACCTAATTGACATTTGCTACACTTTCTCCTTTAGACCTTTTATTTAAGTTG
GGCGGGAACATATTCCTTTTGTTTTCCCCCANATTACCTGGCCNCGGGGCCGGGGCGCTTCTAAAA
AACTAGNTGGGGATCCCCCCCGGGCCTGCAGNGGAATTTCNAATNNTCAAAAGCGTTTATTCGATT
CCCGGCCGACCNTCCCANGGGGGGG

| | |
|---|---|
| Sequence 718 | cMhvSG009d03 |

AGGTACGCGGGGGACATTTTCTCGGCCCTGCCAGCCCCCAGGAGGAAGGTGGGTCTGAATCTAGC
ACCATGACGGAACTAGAGACAGCCATGGGCATGATCATAGACGTCTTTTCCCGATATTCGGGCAG
CGAGGGCAGCACGCAGACCCTGACCAAGGGGGAGCTCAAGGTGCTGATGGAGAAGGAGCTACCA
GGCTTCCTGCAGAGTGGAAAAGACAAGGATGCCGTGGGATAAATTGCTCAAGGACCTGGACCGCC
AATGGAGATGCCCAGGTTGGACTTCAGTGAAGTTCATTCGTGTTCGTGGCTTGCAAATCACCGTNT
GCCCTGTCACAAAGTACCCTGGCCCGGGCGGGNCCGCTTCTTANAACCTAGTTGGGAATCCCCCCC
GGGGNCTGCAAGGGAAATTTCGAANTANTCAAAGCCTTTATTCGAATACCCGTTCGAACCCTTTTG
AAGGGGGGGGGGCCCC

| | |
|---|---|
| Sequence 719 | cMhvSG009h03 |

GGCGAATTGGAGCTCCCCGCGGTGGCGGCCGAGGTACACGGGGGTGGCTGCATGCCAGCCAGACA
CCCAGTCTTGCAAGACTGTCATTGAAAATCTCCGTTTTGCTGTTCTCCGGGTCTCTGCGTCCAGTCT
TTGTGTTTGGACGGACCTGCCGGGCCATCTTTCTGCAAGAAGATAAAGGAAGACCAGGAGTGCCT
GCCGAACTCCTATGGAGGAAGTCTAGGAGAGGAAGGGGACAGGGAGGAAGATGGTGTCTGCAAA
CCAGGAAGCAGCCTTGCCAGACACAGGATTGGCCACAACCTTGACCCCAGACTTCCAGCCTCCAG
AACTGTGAGAAATAAATGTCCATATTGACTAGGGGCACAGGGCATGGGGGAACTGGTTCCAGACC
TGCCTCCTGGGGAAGTTTGGGAGGGGGGCATTTCAACCTGTTAATTTCTCAAATTATGTAGTCATT
CCAAAAAGAAATAGAAACCACCTTCATTTNACTTTGTGATTNGCCAAAATTATTTGGATCAAATTT
CTTCATAAGAAAAGGTTATAACCATTTTTCCCCCTTTTTTGGGTACCCTGCCCCGGGGCGGGGCCG
CTTTTTAGNAAACTAAGTGGGATTCCCCCCCGGGGCTGNGANGGAATTTCCGATTATTCNAAGCCT
TAATCTGATTACCCGNTCCNACCCCTCGANGGGGGGGGGCCC

| | |
|---|---|
| Sequence 720 | cMhvSG010a08 |

CCGCGGTGGCGGCCGCCCGGGCAGGTACTTTTTTTTTTTTTTTTTTTTTTGGTTTAAAATTTCTGGCA
GGTAGAGCAGGTGCCCTCCCCCAGACACTTGCAAAAATGTAGAGAGAGGTGGAGGGCTGGGGTGC
CTGCGAGCAGGTCCCAGTTGCAAGAATTAAAGCCTTGCAACAGGTTGGGGGAAGCAGGGCAGCGC
CAGGTGCACGCAGTGAGCGGAGGCCGGAGAAACCCTCAAGCCTGAGCGGGTCAGAATTATAGGG
GAAAAAAAGCCACAAAATTGTTCACCCCCAAGCAACCACCGAAATAATGAGATCGGATGCAGTGG
AGATGGCGTTGGGGGTGGGAGAGAAAAATGGATTTATCTTTAAAATTTTTGCTTAAAATCTAAAAT
ACACCCCCGCTTTTTAACCCTCAACTTCCAGCGGTGCGCGGCGCCGCANAACAGGTAAGAGGCGTT
NGCTTGCAGCCCNAGAGGGTGGGAGAAAATGTTGAAATTCAAGAATTTNAAAAACNAAAAACCA
AAAACCCAAANNAACCCCCAAACCCNTTAAACACCTTTTTTTTTTCCACTTTTGGCCACCTTCTTTT
TNCGAAAATTNTCAGGTTNTNCGCCAAAANTTCCGGGAAAAAGGGGNGAAAAAACNGGAGGGNG
GGGTTNTTNAAAAAGGGNGCCAAAAAAAAGGGG

| | |
|---|---|
| Sequence 721 | cMhvSG010f10 |

CCGGGCAGGTACCAACAGCCCCTTCCCTCCCAAGTTAGGTGAGCCCTTGGGCCAGTGTATGGGCAG
AAAAGCAGATTTGTGTCCTTCAAAAGGGAAATGTAAAAAAGGTGAAAGCTCTAGTTGAAGGGCAG
TGAGAGGGGCTGGAGTGGGAGAGAAGGTCTCTCCTGGCCGGTGGTCTGGGTGCAGCAAGGGCACT
CTGAGAAGGCAGAATGGAAACGCAGGGCTGGAGGGGCCATGGGCACAGGTTTGGGGGCTCCTTCC
AGCCTCTACTATGTTGCCCCCTTCCCCAAAGCCCTTACAGGGGCCANAAGCCACATTCCCCCGTNG
ACCCTGAGTCTTGGCCTCATTTTGGNGAAAGTCCTTCTGGGGGTGTATTGGGATGCCTGTGTGTTGT
TGAGTGGAAGATGGGTTGGGGGGGGCCAACGGGCTTATCTTGGGCTTCTTAGCACACTTCNATGN
GGGAANAACCCAAGCCTCTTTGGGGAAACAAACAAGGGATTGGGGGGGTGCCTTGGGGGAATNG
GGGGGTT

| | |
|---|---|
| Sequence 722 | cMhvSG012b06 |

GGAATTCAAAATTAACATNCTTGTCCGNGNGCTTNTTNTANACNCCAAAAAAAGTTTCAACCTTGN
GTTCCNCATTGNTCNGCTGNGCTTTNNCCAAAAGAACCTTTNTNAGCCGGTTGCCACCATCAGGAG
GAAAGANCNNAAGGGGNTTTATTTTTTTGCNNAGGNGGTCCATTNNNTTTTAAAAAGNCCCCGNG
GGACCTTGGNCNGCTTTAAAANTANGGGATCCCCCNGGCTGGAGGAANTNTNANANTNAAANCTT
ANTTGGATNCCCGTCGAACCTTTNGGGGGGGG

| | |
|---|---|
| Sequence 723 | cMhvSG012d06 |

TCAGTCCTTCCTTTTATAAGGACAATAATTGGAGTAGTTTAATCTTATTCATGTGCAGATAAAAGA
GGTTTATGAAGTTTAGGGTGAAGTAGGCAAGGGAATCTGTTTACTCCCTCTTCCCTCTACTGAATA
ATTTTCCCTCTACTGAATAATTTTCCCTCTAAGAATTGCTGTGGGTAATACCAGGAGTGGGGACATT
GCCCACATGCATAAGAGCGTATCTCTCCATTCGATCAGTTTGTCACCATCTTTGCTCTGTTTTGAAA
GTCAGGCTTNTCTGTGACTGTGAAGCCCTGCTGTTCCCTGAAAATCTGATAAATGGAGCAGCNGGA
GGGTNTTTTTCTTTCTGGGCTCTNGTANAANCTCATNTGGTGTTGCAACTTTGGTAATTTTCCCAAN
AGTTTGAAAAAGGGAAAGAATTGGAANCTGGGAATAATTGGTGTNAAACCTATTCTTGGCCTTAA
CATTNAGTGGTAGCCATTTTTTGCAAATTT

| | |
|---|---|
| Sequence 724 | cMhvSG012f07 |

GCATGGAGGAATCCACACCATGATCCAATCACCTGCCACTGGGTCCNTCCCTGGACACATGGGGA
TTATGGGGATTATAATTCAAGATGAGAGGAGATTTGGGAAGACCCNCTACATTATTTTGAGACAAT
GGGGAAGCTNAAATGTGCTNANTCGAACCTATTGGGATTTTNAATTTCTCGCCCATTCTTACCAAA
TGTTGATTTTGNTGGGAGGACTTCACTTGTAAACCAGCCAAACCCCTTGCCTAAGGGAAATGGGAA
GAGTTTTGTGCCATAAGCTTCTGGAGAAAAANTGGNAATTGGTGGGTGTTTTTCTCTGGGGGTCCG
ATTGATTCCAGGTAACCATTGTNCAGAANAGAAAAGNTGCCCAAACATGGATTTTGCAATCAAGC
CCCTTTGCCCCAAAAAATNCCCCCCAAAAAAAGGGTTTCTANTTGGGAAGAATTTTGAATGGGCCA
ANGAAAAGNCCCANAATANCTTTTNANGGTTTNCCAATNACTTCGGACTTGTNACCCTTGCCCCGG
GGCGGGGNCGGCTTTTTAGAAACCTAAGTNGGGAATNCCCCCCGGGCCTTGGCANGGAAATTTNC
AATATTNAANGCCTTTTTTNGGATACCCGTCNGACCCTNNAAGGGGGGGG

| | |
|---|---|
| Sequence 725 | cMhvSG012h06 |

GAATTGGAGCTCCACCCGCGGTGGCGGCCGCCCGGGCAGGTACACAGGATTGGGTCTAGACCTTG
ATGCCTGGGTGGAGGGCCCTTGTAAGGGGCCATAGCCTCTTCAGGACCAACTGGAGGGAGAGTTA
GGAAACACCAGCTCCTGCCTGGGGCAGTGAGGGAATGGGAGCAGCTGTGGGCGCCTNATTTNAGG
CAAGTCCTNCCCAAACCTTCAGATGCAGTGAGACCTGGCCTTCCTGTTGTGCTTTTCAGACTTTGTT
TTCAGAATGCTTTTATCTCGAGTGTGCCCTTCGGCCCTCACAAGAGCCCCTGGGGAGTANGTGGTG
GCCTGTGCCGTCATCCCCATTTCAAAGCAGGGAGCTGAGGTCCTGGGAGGGGAAAGTGCTTGCCT
GAGGTCCCACTGTGTTAGTTGGGTGGGCAGGACTNGAACTNGGTTCTTCAACAAGCCCAGAAGCT
NAANTNTTTTAACACCCC

| | |
|---|---|
| Sequence 726 | cMhvSG014d08 |

ACCCCAAGTGTCANCTCCAACTCTTGTNGNGGTCTAANGAAACCTAGGAAAAGTGGNCATCTTNT
GTTGTAAACATCCTGAAGCAAAAAGAATGCCCTGTGCANGAAGACTATCTATCCGTGGTCCTGAA
CCAGTTATGTGTGTTGCATGAGAAAACGCCAGTAANGTTGACAGAGTCACCAAATGCTGCACAGA
ATCCTTGGTGAACAGGCGACCATGCTTTTCAAGCTCTGGAAGTTCGATGAAACATTACGTTCCCAA
AGAGTTTAATGCTGAAACATTTCACCCTTTCCATGCCAGATATATTGCNCCCTTTTTGNAGAAGGG
AGAGNACAAAATCAANGAAAACAAACCTGCACTTTGGTTTGGAGCCTNCGTGAAAACACCAAANG
CCCCNAGGGCAACCAAAAAGGAGCCAACCTTGGAAAGCCTTGTTAATGGGATTGGATTTCCGCCA
GCTTTTTTGTTANAAGAAAGTTGCTTGCTAAAGGCTTGGACCGATTAAGGGAGAACCCTGCTTTTG
GCCCCGAGGGAGGGGTTAAAAAAAAA

| | |
|---|---|
| Sequence 727 | cMhvSG014g05 |

TTGGAGCTCCCCGCGGTGGCGGCCGGCACCTTGGCCGCNTTCAGAGTGCCNATGAGCTCCNNCNG
ANANGGNTTCCGCCNNAACAANNNACNTTTTNCCCCAACGAAGAACTTCCTGGAGGGCGCCATGG
CGCTGGAGCCNAGGTGCTTAAGGTCAGTGTCTCCCGCGTACCTCGGCCGCTCTAGAACTAAGTGGA
TCCCCCGGGCTGCAANGAATTCCATATCAA

| | |
|---|---|
| Sequence 728 | cMhvSG014g05 |

TTAGTGAGGGTTTAATTGCGCCGCCTTGGGCGTTAATNNATGGTTCAATAAGGCTGTTTTTCCCCTG
GTTGTGAAAAATTTGTTTANTTNCCNCTTCCACAATTTTTCCACAACCAAACCANTACCGANGCCC
CCGGGGAAGCCATAAAAAANNTNGTTAAAAAANCCC

| | |
|---|---|
| Sequence 729 | cMhvSG015b06 |

ACCCNGCTCCACCGTGGTGGNTGCCNCCCGGGCAGGTACACTGGTGATTTCTCAAGACAAGAAGA
TAGGCACTTAATGGCAACNTGAAATTCCTAATATTAAGCCTGATATTCTTATCATTGAATCTACTTA
TGGGACCCATATCCATGAGAAACGTGAAGAGCGAGAAGCAAGATTCTGTAACACTGTCCACGATA
TTGTAAACAGAGGAGGCAGGGGTCTCATTCCTGTCTTTGCTCTTGGAANGGCTCAGGAGCTGCTCT
TGATTCTAGTATGAAGTTACCTCGGCCGCTTCTA

| | |
|---|---|
| Sequence 730 | cMhvSG015b06 |

GAATTCCGATATCAGAGCTTTATNGATACCCNNCAGNCCCTCGNAGGGGGGGGGCCCCGGGTTCC
CCAGCCTTTTTGTTCCCTTTAGGTTGAGGGGTTTAATTGCCGCGNCTTGGGCGTAATCATGGTTCAA
TAAGCCTGGTTCTNCCTGGTGGTGAAAATTTTGNTTAATTCCCGNCTTCACANATTTTCCCACCACC
ANACCANTTACCNANNCCCGGGGGAAGCCANTNNAAANGTGGTANAAAGCCCCTGGGGGGGT

| | |
|---|---|
| Sequence 731 | cMhvSG015b12 |

AGGACGCGGGGGCATTGCCGAAGTGGAAAATGATGAGATGCCTGCTGACTTGCCTTCATTAGCTG
CTGATTTTGTTGAAAGTAAGGATGTTTGCAAAAACTATGCTGAGGCAAAGGATGTCTTCCTGGGCA
TGTTTTTGTATGAATATGCAAGAAGGCATCCTGATTACTCTGTCGTGCTGCTGCTGAGACTTGCCAA
GACATATGAAACCACTCTAGAGAAGTGCTGTGCCGCTGCAGATCCTCATGAATGCTATGCCAAAGT
GTTCGATGAATTTAAACCTCTTGTGGAAGAGCCTCAGAATTTAATCAAACAAAATTGTGAGCTTTT
TGAGCAGCTTGGAGAGTACCTGCCCG

| | |
|---|---|
| Sequence 732 | cMhvSG015b12 |

AGCTGTTTCCTGTGTGAAAATTGGTTATCCGGCTCACAATTTCCACACAACATTNCCGAANCCGGG
GAGGCATTAAAGNGNTAAAAAGCCCTGGG

| | |
|---|---|
| Sequence 733 | cMhvSG015h02 |

CGAAAACTGATCAGACTGTCTCAGATCAAGGAAAAGATGGCCAGAGAGAAGCTGGAAGAAATAG
ATTGGGTGACATTTGGGGTTATATTGAAGAAGGTTACGCCACAGAGTGTGAATAGTGGAAAAACC
TTCAGCATATGGAAACTGAATGATCTTCGTGACCTGACACAATGTGTGTCCTTGTTCTTATTTGGAG
AAGTTCACAAAGCGCTCTGGAAGACGGAGCAGGGGACTGTCCGTAGGGATCCTCAATGCCAACCC
CATGAAGCCCAAGGATGGTTCAAAGGAGGTGTGTTTATCTATCCGATCATCCTCANAAGGTCTTAA
TTATGGGTGAAGCTCTTGACCTGGGAACCTGTANAGCCAAAGAAGAAGAATGGAGAGCCGNGCAC
CCAGACTGTGAATTTGCGTGACTGTGAGTACCTCGGC

| | |
|---|---|
| Sequence 734 | cMhvSG015h02 |

AGGAAATTCGATATCAAGCTTTATCGATACCCGTCGANCTNGAGGGGG

| | |
|---|---|
| Sequence 735 | cMhvSG027b09 |

CCACCGCGGTGGCGGCCGCCCGGGCNGGTACNCGGGGGGCACCANCACTTGGAGATTTTTCCGGA
GGGGAGAGGATTTTCTAAGGGCACAGAGAATCCATTTTCTACACATTAACTTGAGCTGCTGGAGG
GACACTGCTGGCAAACGGAGACCTATTTTTGTACCT

| | |
|---|---|
| Sequence 736 | cMhvSG027b09 |

ACCCAGCTTNTTGTTCCCTTTTAAGNGGANGGTTAAATTGCGCGCCTTGGCGTAATCATTGNGTCA
TTAGCTGNATTCCCTGNNGTTGAAAANTTGTTTATCCCGCTCACCAATTTCCACAACAAACAATAC
CNAGCCCGGGG

| | |
|---|---|
| Sequence 737 | cMhvSG027g03 |

GATTGAGCCCTGGCAGGCATATGCATGCAGCACTGCCTACACAGTCCTGAGTCANAAACTTCTCAT
GGGGTCTCTGAGTCTGGAATGTCTGAGTTCTCAGGAGGGGTAGCATTTGCTGCTAACCCTCTGCCT
CCTTAGCTTGAGCTGTCTNTCGNGGTTTTTTCCCCTGATGGATGTTAACATCTTCCCAACAGAGCTN
TCAACCCAGTGAGGGAGGAGTCTGTGTANATCNCCTCCCATCATTCTCCATANAGTCTNTNTGGCC
CAGGTTAGAANAAAAAGACTTCTTGGCTCANACTCCAAAGACTANAGTCAGGGACAGTTTCCTTA
GNGGTGTAAAATGGCAAGAGTAGCNCTAATCTCACAGAANACTCCTGCANAACACACTGGCACAT
TTCAACCATNAAGCTGNTCTCAACAGTGTGAAGCCTGGGCAAGCACTTCCCCCTTTTAATGGTTNG
ACCTTTNGAAAAAATCTNNATNTGNNNGAGCCCAACCAGGGGAAAGACCCTTNTTGCATTTCATT
NCCCTGGACTCCTTTCAANAAAGCNANGGGCNAAAACCCTTTTTTTT

| | |
|---|---|
| Sequence 738 | cMhvSG027g12 |

GGGCGAATTGGAGCTCCCCGCGGTGGCGGCCGCCCGGGCAGGTACATTGCAGATCCCAACATTGC
TAAGCTTGTTCACTTTCAGGGTTATCCATGTGAACTTTTGCCTCTGACGGTCGCAGGTATTCCATCT
ATGCACATCTGTCTAGATTTCATACCTGAGCTTATTGCACAGCCAGAACTTGAGAAACAGATATTT
GCTATCCAGTTGCTTTCTCACTTGTGTATACAATATGCATTACCAAAGTCACTTAGTGTGGCTCGTT
TAGCTGNCAATGTCATGGGAACTTTGTTAACAGTTTNAACACAGGCTAAGCGGTATGCTTTTTTTA
TGCCAACTCTGCCAAGTTTGGTCTNTTTTTGTCGAGCATTTCCTCCATTGNATGAGGATATTATGTC
TTTGCTGATCCAAAAAGGGCAAGTTTGTGCCTCTGATGTTGCCACTCAGACAAGAGACATTGNTCC
AATTATTACACGNTNTTCNACAAATANAAGGAGAAACCAAGTGGGATGGNCTCAAAATCTGGTAA
AGATTCANTCTTTATAAAAATGGANNCAAGGGACCCCTGGAAGCATGGGANTCCCTGAATGNACC
CTCGGGCCGGNTCTANNAACTAAGGGGGAGCCCCCCNGGCTTGCAAGGAAATTCCGNTANTCAAA
GCTTNNTCCANTANCCGTGGGNACCTTNGGAGGGGGG

| | |
|---|---|
| Sequence 739 | cMhvSG028b10 |

CCGGGCAGGTNCGCGGGGGCATTGCCGAAGTGGAAAATGATGAGATGCCTGCTGACTTGCCTTCA
TTAGCTGCTGATTTTGTTGAAAGTAAGGATGTTTGCAAAAACTATGCTGAGGCAAAGGATGTCTTC
CTGGGCATGTTTTTGTATGAATATGCAAGAAGGCATCCTGATTACTCTGTCGTGCTGCTGCTGAGA
CTTGCCAAGACATATGAAACCACTCTAGAGAAGTGCTGTGCCGCTGCAGATCCTCATGAATGCTAT
GCCAAAGTGTTCGATGAATTTAAACCTCTTGTGGAAGAGCCTCAGAATTTAATCAAACAAAAATTG
TGAGCTTTTTGAGCCAGCTTTGGAGAGTACCTNGGCGCTCTAGAACTA

| | |
|---|---|
| Sequence 740 | cMhvSG028b10 |

GCCCGGTACCCAAGCTTTTGTTCCCTTTAGTGGAGGGTTAATTTGCCGCCGCCTTNGGCGTAAATTC
ATGGGTCATTAGCTGGTTTTCCCTGTGGTGGAAAATTTGGTTTATTCCCGCTTCACCAATTTCCCAC
CACCAACCANTACCGGAAGCCCCGGGGAAGCCATTAAAAGTTNGTAAAAAGCCCTNGGGGGTGGC
CCTAAATGGAGGTGGAGCCTTAACCTCACAATTTTNAATTGGCGGTTTGCCGCCTCACCTTGGCCC
CGCCTTTTCCCAAGTCCGGGAAAACCCTGGTCCGNNGCCCAAGCCTGCAATTTAATTGGAAATTCG
GGNCCAACCCCCC

| | |
|---|---|
| Sequence 741 | cMhvSG029c10 |

GAAGTGGCGCCTCTGAGAAAAGAAGGTTGGAATTATCGTAATTTGTTTCTAGGCTGAGATACCAGC
ATGGAGAAAATGTTGGAGTGTGCATTCATAGTCTTGTGGCTTCAGCTTGGCTGGTTGAGTGGAGAA
GACCAGGTGACGCAGAGTCCCGAGGCCCTGAGACTCCAGGAGGGAGAGAGTAGCAGTCTCAACTG
CAGTTACACAGTCAGCGGTTTAAGAGGGCTGTTCTGGTATAGGCAAGATCCTGGGAAAGGCCCTG
AATTCCTCTTCACCCTGTATTCAGCTGGGGAAGAAAAGGAGAAAGAAAGGCTAAAAGCCACATTA
ACAAAGAAGGAAAGCTTTNTGCACATCACAGCCCCTAAACCTGAAGACTCAGCCACTTATCTCTGT
GCTGTGCTAGGAAACAATGCCAGACTCATGTTTGGAGATGGAACTCAGCTGGTGGGTGAAGCCCA
ATATCCAGAAGCCTGACCCTTGCCGTGTACCTTGCCCCGGGGCGGGNCGCTCTAGGAACTNGTGGG
ATCCCCCCNGGCTTGCAGGGAATTTCNAATATTCAAAGCCTTATTCCGATNACCCGTCGACCCTNC
GAGGG

| | |
|---|---|
| Sequence 742 | cMhvSG038b09 |

CCNGGCANGTACCTGCACGCCTGCNACACCNACCTCTNTCTGGGCNTNTATTACAACCNAANATN
ATNTGGNTNTGNAAGGCGCNAGCCACTTNTTCCNNNAATTGNCCGATGANAANCCCNNGGGCTAC
NAGCGNNTCCTGAANATGCAAAACCAGC

| | |
|---|---|
| Sequence 743 | cMhvSG038b09 |

GCCATTAGCTTGAATTCCTNGNGACGACAATTGGGTAATAGCGGCTCAACAGATTTTCCTACACGA
ACCATTACTNAGCCCTTGGGCNGCNATAAAAAGTTNGTCTANAGCCTNTTGGGGTGTTGGCCCTAN
ATCGGAGNTTGGAAGCCTAAAACTCCAGCAATTTAAAATTT

| | |
|---|---|
| Sequence 744 | cMhvSG038f03 |

CGCCGGTGGCCGGCCCCNGGTACNCTGGNTGCNNCCTACTANTNGCCATATTGGCCCGTGGGGNG
GNGGGGGGGGGACTCAAAAAANAAAANAANTNTTTTTTTTNNTTCCCTGNANGACCACTGGNAAG
GTCAAGCTCAGAATCTATTACTNANAGAATTTTTCCCTGCNCATNTATGGTNTCCCCANCNACTCN
ANNGATTNACTAATTAATGTAACTTTGTTNAAAAAAA

| | |
|---|---|
| Sequence 745 | cMhvSG039d11 |

TTGAAAGAGGAAAATCTGTGGCCAAATTCAAGGCACCCTAGGCTGTGATCCTNGNACTGAACATC
TNGATGAGTCAATACAGGGCACGGAGTAGGACTTTGAAGTCCTCCATTGGATCTTCTCGGANGATG
ANGGAAATGAGAGAGTGTGGAGA

| | |
|---|---|
| Sequence 746 | cMhvSG040c02 |

TGCATAGACTAGTCAGCTTCTGGGGTGACTAGAGCAGGGCTGTTGTCTCCTCAAGCTTCAGCCGTG
CGTGGACTGGTCAGCTTCCGGAGTGACCAGAGCAGGGCTGTTGTCATCTCACTGGCACCTTGGTTC
CATCGTAGGATCAGCTGGGTTGCATGGTCTAGGTCCTGTTGGCTGGTCCACTTGTCCTGGGCTGCT
GGTTTCAGCTGACTGGATGGATGGATCCAAGGCACAATTCCTGCAACATTTCTAGGCTTCCAAGTG
GGTCCCTGGCGTCTTANCTGTGGGATCTCCCAATACCTGCAGGTAAACGAAGGCCCACANGAAGC
CTGGGCCCTCTAGGGAGCCAGGAAAGACACAGTAGCCAGTTGAAAGACTACACCCAAGAAGCCTC
CCGGCTTGCCGCCAGAAGACAAAAGGCCCCGCCCCCCCGCGTTACCTTCGGCCGCTTCTTANAAAC
TAAGTGGGGAATCCCCCCCGGGGCTTCAAGGAAATTTCCGAATANTCAAAAGCCTTATTCCGAATA
CCCCGTCCGACCCTTCGGAAGGGGGGGGGGCCCCCGGTACCCAAACTTTTTT

| | |
|---|---|
| Sequence 747 | cMhvSG041a07 |

TTTTTTTTTTTTTTTTTTTTTTTTTTTTTTTTTTTTTTTTTTTTTTTTTTTTTTTTTTTTTTTTGGGNNNCN
NNCNTTTAAAAANCNNNGGGCNNNAAGGGNTTTNAAGGGTTTAAANCCAANNANCCCATTTTTTT
AANNTTTNNANNCCCNNGGNTTAAAAAAACNNAATTTTTNNAANAATTTTNGGGCAAANNNACNN
CCNTTTTCNAAAAANNGTTTTCCNCCNGGNNNNTTTCCGGGCATTNCTTTTCCTGNTTTTNAAAGG
GCTTNTTNTNNAAAAAAAAAACNTTNCCCCACNATGGATTCNANGGGGNTTAATTCCCCCCCGCTT
NNNGGNNCCTTGGGTNGTCCCCNAAAATNNGNGCCCCCAAAAATCCCNGGGGNNNNTNGGGGGT
GGNTNNNTGGGGGNAAAAATNGTNNTTNTNNNANCCTAAAAATCTTTTTTTTAAAACNTAAAGGG
NCCCCANNTTTCTTCTGGGGANNNAAAAAAAANCCCCCNCNNANNANANNAANNNGGGNTTTTTT
TAAAAANNAAAANANAAGGGCCCCNTNTNTNAAAANNTTGCAAAAAAAAANGNAANNNGGGGG
NGCAAAATTTCCNTTTNTNGGGGGGGGGG

| | |
|---|---|
| Sequence 748 | cMhvSG043d02 |

TCNCCTTTACTTGGAAGGCNNCCCTNGNGNNGGACCCATNCATGATTCAGATCACCAGNAAGGGN
GNCCTNCNCTCNNTTNTGGACATGCATGTCAACGTTGGTGGGAGAAGCTATGTGCCGGGAAAAAT
GAAAGGCAGAAAGGCCAGGAGGNCTGTANNGCCCTNANACATGGCCAAGAAAACTTTCAACCCC
ATCCAANCCATTGTGGACAACATGGAATGTGNAACCAAANTCCAAAACAAANCCATGATTTNNCC
TGCTCCATTGGGNGACCCNACTTGTGCNTTCGGNAACNCTGGNCATANCAGGAACCTCGTGGAAA
TTTNNNCCNCGTCTNTTNNANTNANTGCCCCTGANANNACNGTNGCNATNNNTATCTNCNGNGCN
TTTGCCNCCCNATTCCNTTCGGGNNTNTTCTTATTCCCAAATCCGGGNNAGGGAAGAANTTGGCTT
TCTTTNTNTACCCACTTGNTCCCTGGAGGGCANCCCCCCTTAAGAAAGCTTTANGGGAACGTTNAT
TTCTTTGACNCANGTNGGGNTNNNNANCCCAATGCNTNTTTGAACCCTTTTTNGNTTNNNCNTGTG
GTTNGGGCCC

| | |
|---|---|
| Sequence 749 | cMhvSG045f03 |

AGGTACGCGGGATNGANAGGTTGACCNTGTGATACCGCGGGACAGTTCACATAGACATCAGANAA
TTTATTCCAGAAAGGAGCCTCCTGAATGTGATGAATACGGCAAAGCCTTTAATCACATCTCAGCCC
TTAGCATCGGAAAGCTTATACTGTAAATAAACTTGATGAATATTATATGTGAGGAAAACTTTCATG
TATAGCACTCATTGCTTCANACANAAAATGAATTCCGTCGGTATGTTCCAATCTGTGATGAAATTT
TGAGNAAACATTGCCAAGGAGGGAGCTCAATCTTGNGCCGGGCGCAGTTGGGCTTCACGCCTTGT
AATCNGCAGCCACTTTGGNGAGNGCCCGAGGGCATGGCGGGATCACCGGAGGTCAAGTTTGTTTC
GGAAGGACCCAGCCCTNGGNCCAACAATGGGTGGAAACCCTTGTCTTCTTACTTGTGNANACAAN
GATTNATGTNANAACATTATTTATAANGGGTNCCCTGCCCCGGGGGCCGGGCCCGNTTCTTAAAAA
AACCTTAGGTNGGGAATTCCCCCCCCGGGGGCCTTGGCAAGGGAAATTTCCNAATTTTNTCCAAAG
GCCTTTNTTTCGGAATNCCCCGTTCCGAACCCCTCCNAAGGGGGGGGGGGGNCCCCCGGGTTCCCC
CAAANCTTTTTTTGGGT

| | |
|---|---|
| Sequence 750 | cMhvSG050b10 |

TTTTTTTTTTTTTTTTTTNTTTTTCCCCAACAAAANCNGNTTGNTTTTNTNGCNGGGAACCTGGGAN
GGAATNGGNCANCNGGGGGTNNCCGNAGNANCCCCNTCCCCCGGCNTGACTGCCAANNCCCAGN
TTTGTNTGNAACCCAGNGGNNGGATCANNNTCCNNCCCCNTTNGGNCCATCCNGGGGGNNGGGGG
GACCANNCCCNTNTTTNTNANGGCCANGGGNGNAAACAGTNTTTCCNGTTTTTTTAAGGGTTGCAA
NCAAAGNGCCCATNCTGGGCNAAAATTNAANGCAANCCTTTTTGNGGGGCNGGNNAANGTNATNC
TTAACNCCCCCAAGCTTNTTGGGGNCCCGANAAACAGTTTAAANNNANCNTCCANAGGTNNTNTC
CNNAAAAAACTCNNNCTTNGGCNNAACTGAGGCANCGGCGTTTTTGGCCNCTTTTTTNGCGGNNG
TTTAAAAAAAACNCNTTTTTTTCCCCGGGTACCTTGGGGCNGGTTTTAAAAANTTNGGGGGGATCC
CCCNGGGNNTGGNGGNAATTCNNTTTNAAAGGTTTTTTGGNNCCCCCGCNANCCTGNNGGGGGGG
GGC

| | |
|---|---|
| Sequence 751 | cMhvSG051f01 |

AGGTACAACATTGGTGTCCTAAGACACCTTCAGGTCATCTTTGGTCATTTAGCTGCTTCTCGACTGC
AATACTATGTGCCCAGAGGATTTTGGAAACAGTTCAGGCTTTGGGGTGAGCCTGTTAATCTGCGTG
AACAACACGATGCTTTAAGAATTTTTTAATTCATTGGTGGGATAGTTTAAGATGAAGCCTTTAAAA
GCTTTTAGGGACATCNCAGGCTATGCTAAGGTAAAAGNTCTTANGGANGGTTTCCTTTTGCCTGNA
TCAGNAAGGAATCTTGCNCATAGGGCTTGNCCCCACATTNGGTACCNTGCNCCNGGGGGCCGGGC
CCGCCTCTTAAGAAACCTTAGGTTGGGGATTCCCCCCCCCGGGGCCNGGCCNANGGAAANTTTCN
GGNATTATTCNAAAAGCCTTTAATTCCGGGATTACCCCGNTCCTGAACCCNTTNGGAAGGGGGGG
G

| | |
|---|---|
| Sequence 752 | cMhvSG052a08 |

CCGGGCAGGTACCACCTCAACATTTCCTTGTGCTGAAGCTATACTGAGGACTGTCCTACCTTCACT
ATCAATACTATCCACAGCTGCACCCCAAAACAAAAGTGTATTTACAACTGATGCATGACCCATAGA
CGCTGCTGCTAAGAGGGGTGTACCT

| | |
|---|---|
| Sequence 753 | cMhvSG052a08 |

GTTAAATTGCCGCCGCTTTGGCGTTAAATCATGGGGCATAAGCTGGTTTCCTGTGGTGGAAAAATT
NGTTAATCCCGCTTCAACAAANTTTTCCCACAACAAAACCATTANCGAAGCCCCGGGGAAGGCCA
NTAAAAAGTGGTTAAAAAGCCNCTTGGGGGGGTTGGCCCCTAAATTGGAAGTTGAAAGNCCTAAA
CCTTCAACANTTTAAATTTNGCCGTTTGGGGGCCTTCAACCTGGGCCCGGCTTTTTCCCAAAGTTCG
GGGGGAAA

| | |
|---|---|
| Sequence 754 | cMhvSG052f04 |

TCCACCGCGGTGGCGGCCGCCCGGGCAGGTACGCGGGGGCATTGCCGAAGTGGAAAATGATGAGA
TGCCTGCTGACTTGCCTTCATTAGCTGCTGATTTTGTTGAAAGTAAGGATGTTTGCAAAAACTATGC
TGAGGCAAAGGATGTCTTCCTGGGCATGTTTTTGTATGAATATGCAAGAAGGCATCCTGATTACTC
TGTCGTGCTGCTGCTGAGACTTGCCAAGACATATGAAACCACTCTAGAGAAGTGCTGTGCCGCTGC
AGATCCTCATGAATGCTATGCCAAAGTGTTCCGATGAATTTAAACCTCTTGTGGAAGAGCCTCAAA
ATTTAATCAAACAAAATTGTGAAGCTTTTTTGAGCAGCTTGGGAGNAGTACCTCGGCCCGCTCTAA
GAACCTAGTGGGAATCCNCCCGGGGCCTGCAAGGGAATTTNCGATATCAAAGCTTTATCGAATAC
CCGGTCGACCCTCNAAGGGGGGG

| | |
|---|---|
| Sequence 755 | cMhvSG053g11 |

TTCCAAGGCCCTGNGGGGAAANTTNTTATTAATTCAANTGACAAAATTTGTGTTAAAGTGGCCTTC
TTTTAAGGNACAGACAATAGTNAANACCTTGACTCANGAGGCTGTCTTCCTTGGGGAGACTNTTGG
CANAACATGAGCATTGACCAGAATTTCAAAGGGAAAGGGGCANGGACCGGGGGGCTCTTAAATA
AAAGAAGGGGGAGGGTTNANNTTNGTTTAATTGGNGCCATTNNTNCAGGGAAGGGGTTGAAAGA
ATAACCTTCNCCCCCCAGGGGGGTCCTCCAAGGGAAAGGGGCTTGGGGGGNGCCTTTTGGTTANA
AAAACCTTGANGAATGGTGGCCAANGGAAGAAGAAACCATTCTTTNTTAAAANAAATGGGCCATT
GCCTTTGGGGCTTGGNNCCGCCAANTTGGGGCCTTCAACCACCCCTTGGTAAAATTCCCCAAGNTG
TTGTTTCCCCGGG

| | |
|---|---|
| Sequence 756 | cMhvSG069e08 |

TTGACCTGCTAATCAAGNCACACATGGTGAGCGNGGACTTTCCGGAAATGATGGCAGAGATCATC
TCTGTGCAAGTGCCCAAGATCCTTTCTGGGAAAGTCAAGCCCATCTATTTCCACACCCAGTGAAGC
ATTGGAAACCCTATTTCCCCACCCCAGCTCATGCCCCCTTTCAGATGTCTTCTGCCTGTTNTAACTA
TGCACTACTCCTCTGCAGTGCCTTGGGGAATTTCCTCTATTGATGTCCTCGGCCGCCCGGGCAGGTA
CCCCGGGGGACAGATNCTATTATTATTTCCATTCTACCGAGAAGGAGACTAAGGCTCTGATCATTT
AAATNAGTTGCCTAAGGTGATGCANTGATATAAGTAGCAGAGCTAGGAATTGAGCCTTGGTAACT
TTAACTCTGGACCCNAAGTCCTTAGCTACTAAGCTTTTACTGCATGGGGTTTTNAGTCANAATTAA
AAAACTTTTTTGGAATATGGAGGGTAACNTTTTTGGNGAATTAGCCTTTTGGTGGNTAATTTTNTTT
GNGCCTNATTTGNCCCAACAAAAGNCTAATTTTTATTT

| | |
|---|---|
| Sequence 757 | cMhvSG070g11 |

ACCAGCCTTTGGGAAGTCGTGTGAATACCTCGGTCTCTTAGCCACAGGGATAGAATGGCGGCCTGA
CGGAGCCGCGGCGCCGGCGAAGTCGCTGAGGCGCGAGCTGGAACCCCCAGACCAGCTCAAACGG
GAGCCAAAACTCGAAGCTTGGAAGAATTAGCAGGAAATGGCGGATGAGGCGTTGTTTTTGCTTCT
CCATAACGAGATGGTGTCTGGAGTGTACCTCGGC

| | |
|---|---|
| Sequence 758 | cMhvSG070g11 |

CGCGCTTGGCCGTAATCATGGGTCATAAGCTGTTTCCTGTGGTGAAAAATTGGTTATCCC

| | |
|---|---|
| Sequence 759 | cMhvSG072g01 |

ACCATAGTTGAAGTCTTCAACAATCCCATTAAACTTCAAGCAGAATGGCCTCCACTTCTCTTTGGCT
GATTCTGACTTGAGTTCTTCTGGGTCCAACACATCTATCCTAAGGGTCTCAAAATTTTTCCGGAACT
CAGAGTAAATTTGGTCATCTACTTTGGTGAGTTTCAGGAACTGTGGGTCAACTGATGAAATCAGCT
TGTAATAGACTTCAGCATGCTGCATTGCTCTCATGGCCCAAGCCATCTCAATGTCAGGATCGTTGC
CATACGACTCTGCTGGGAGAGAAAGCGCATGTGCCACAGACACCAACTCCCCGGAAACCGGCTCA
TCAGTTCCACTGGTGGCCGCCATCTTGCAACCCCCGAAAGCGTGGCTCCTTCCGCAGCTGATTGCC
CGCGT

| | |
|---|---|
| Sequence 760 | cMhvSG072g01 |

CGGGCTGCAGGAATTTCGATATCAAGCCTTATTCGATACCGTCGACCCTNGANGGGGGGGCCCCG
GTACCCCANCTTTTTGTTNCCTTTTAGTTGAGGGGTTAATTGCGCGCTTTGGCGTNANTCAATGGGG
CATAGCTGGTTTCCTGTGTGAAAAATTGGTTATTCCGNTCNCAATTTCCACAACAANCATACGAGN
CCGGGAGCATAAAAGTNGTAAAAGCCCTNGGGGTGGCCTTAATGAGGGGNGCCTTACTCACAATT
AAATTTGGGGTTGGGGCTTNNTGCCCCNCTTTTTCAAGTCCGGGAAAACCNTNTNCGTGCCCNNCC
TNGCATTTAANTGAATTNGGGCA

| | |
|---|---|
| Sequence 761 | cMhvSG073g03 |

TCGAGGTACTTGTGACAGGCAGACGTGATTGCAGCCACGAACACGATGAACTCACTGAAGTCCAC
CTGGGCATCTCCATTGGCGTCCAGGTCCTTGAGTAATTTATCCACGGCATCCTTGTCTTTTCCACTC
TGCAGGAAGCCTGGTAGCTCCTTCTCCATCAGCACCTTGAGCTCCCCCTTGGTCAGGGTCTGCGTG
CTGCCCTCGCTGCCCGAATATCGGGAAAAGACGTCTATGATCATGCCCATGGCTGTCTCTAGTTCC
CGTCATGGTGCTAGATTCAAGACCCACCTTCCTCCTGGGGGGCTGGCAGGGCCCGAGAAAATGTCC
CCCGCGTACCCTGCCCGGGGCGGCCCGCTTCTTANAANTAGTTGGATCCCCCGGGCTGCAGGGAA
ATTCGGATATCAAAGCTTTATCCGATACCCGTCGACNCTNGAGGGGGGGGCCCGGTACCCAAGCTT
T

| | |
|---|---|
| Sequence 762 | cMhvSG078h09 |

AGGTACTTGTGACAGGCAGACGTGATTGCAGCCACGAACACGATGAACTCACTGAAGTCCACCTG
GGCATCTCCATTGGCGTCCAGGTCCTTGAGCAATTTATCCACGGCATCCTTGTCTTTTCCACTCTGC
AGGAAGCCTGGTAGCTCCTTCTCCATCAGCACCTTGAGCTCCCCCTTGGTCAGGGTCTGCGTGCTG
CCCTCGCTGCCCGAATATCGGGAAAAGACGTCTATGATCATGCCCATGGCTGTCTCTAGTTCCGTC
ATGGTGCTAGATTCAGACCCACCTTCCTCCTGGGGGGCTGGCAGGGCCCGAGAAAAATCCCCGCG
TACCTGCCCG

| | |
|---|---|
| Sequence 763 | cMhvSG078h09 |

ATTGGGTATCCCGGTCACAATTCCACACAACATACCGAGCCCGGGANGCATAAAAGTGGTAAAAG
CCTGGGGTGCCTAATGAAGTGAGCTAAACTCACATTAATTTGCGTTGGCGCTTAACTGCCCGCTTT
TCAAGGCNGGGAAACCTNGNCCGNGCCCACCTNGNATTNAATGAATCGGGGCCAACCCCCCGGGG

| | |
|---|---|
| Sequence 764 | cMhvSG023h11 |

ANCACCATTCTTAGNGGAGCANGATTCTTGAT

| | |
|---|---|
| Sequence 765 | cMhvSG040e03 |

TCCACCGCGGTGGCGTCCCAGCCACTCAGGAGGCTGAAGTGGGAGGATCGCTTGAGGCCGGGATT
CGAGGCTGCAGTGAGTTGTGATCATGCCACCACTGCTCTCTAGCCTGGGCAAGAGTGAGACTCCGA
CTCAAGAAGAGAAAAAGAAAAACCTTCCAGGGGCACATTTATTTGTAAACCATTCCAGAGGATAG
AAAAGAGATGTAAGGCTCCCTAATTCATTCCATACGGTTAGCGTAATCCTTATAGCAAACTGCACA
AATAAAACACAAGGAAAACTAAACCAAATTCAATTAATGTAGGTGCAAAAAATCCAAAATAAAA
CTAGCAGTTTGAATTCAGCATTGTAGCAAAAGATATATCATTTTCAAGGAAGATTTGTACCT

| | |
|---|---|
| Sequence 766 | cMhvSG052d02 |

ACCNCGGTGGCGGCCCGAGGTACAGTGTCCATGTGTNTACCTGATACTTTCACATGTCATNAAANT
NNANGCANCCAGACACAAGTAGCCATGNATCTTGGCACAT

| | |
|---|---|
| Sequence 767 | cMhvSG064e10 |

CTCNTATAGGCGAATGGANCTCCCCGCGGTGGCGGCCGNGTCCTTTTTTTTTTTTTTTTTTTTTTT

| | |
|---|---|
| Sequence 768 | cMhvSB024h11a2 |

CCCTTAGCGTGGTCGCGGCCGAGGTACTGATGGGACAGCAGCCAGTGCCACCGTGGCCATAGCAG
GTATCCATTTCCAATGGTATAACTTGTCTGCCTTGGAGCAGCACATTTCTGATGCCCTGGGTCAACA
TTTCAGATTGTAATGAATGTCAAACAACTGTTACTGAGATTCTTGTCTGATATTCCCTACACCTTTT
TTCTAGAGAGGAGCATACTCCAGTATTTTGATTATTCTCTTCATAAAGGATGGGATATGCTCATTTC
ATCTATTCAAATTTTTAGATTAACTTAAGATAGCTAAAAATTTAAATATCTAAAATGCTGCCAAAA
TAAAAGAGAAAACACATTTGGCTTTACTCTCTCAACTTTGTATGTGAGAGAGAACATTCCTGTGTT

| | |
|---|---|
| Sequence 769 | cMhvSB026e02a2 |

ACCACAATCACAAATGCAGCACTGTTTACTGACAGGACCATTACTCTGTCAAAATCAGCACATCAA
AAATATTATCCTGGAATCTAAAATAGTAGTCAACTGGGTTGTTAAAGCAAGGGATTGCTATAGATC
TACAGGACAAAGTTCCATAGTGAAACACAAACTCCTGGGTTAGTCCTAGGCCAGGCAGGTGACCA
TAAATGTTCACATTCTGGTAGAATCCCATTTTCTAAAAATTATACAAACACATCGAAATCACTAGA
TTTTATATATATATACACACACACACACTTATGTGTATATATACATATACGTATTTTGTGTGTGTGT
GTTGTGTTTCCAGCAGCTAATAGCAGCTAACATTTATTGAGCACTTACCACATGCCAGGA

| | |
|---|---|
| Sequence 770 | cMhvSB026e03a2 |

CCCTTTCGAGCGGCCGCCCGGGCAGGTACTCAGCTGGCTGCATCACTTATTTTCCTTTCAGACCTGT
CTCCTGTAGGTAGCCATGCTTGTGTCCCCAAAACTATACTGTCTTCCTAATCTTTTCTTCCAAATGA
AAATCGACCACCCAAACCCAAATTTCTTAAGCAGGTTACAAAAATGTTTAAACCAAGTTATATATA
AACTGCAGTCATATTCTCCAGAAATACAAATTAATATGGCATCTAGTTTACTCCCTCTCTTTGGACC
CCAGTTCCACCTTGCTTTCACTCTCACAGGCTTTCTCCTTGGCAAAGCAAATTTAAGAATGAAACTC
TATACACAACCTCTTTTTTCAATGGTGCTACTGTATTCCCCTCTTCAAGGGTTAGAGAGTTTTTCTA
C

| | |
|---|---|
| Sequence 771 | cMhvSB026f01a2 |

CCCTTTCGAGCGGCCGCCCGGGCAGGTACACGTGTGCACGCACATGCACATGAACACAGGAATGT
TCTCTCTCACATACAAAGTTGAGAGAGTAAAGCCAAATGTGTTTTCTCTTTTATTTTGGCAGCATTT
TAGATATTTAAATTTTTAGCTATCTTAAGTTAATCTAAAAATTTGAATAGATGAAATGAGCATATC
CCATCCTTTATGAAGAGAATAATCAAAATACTGGAGTATGCTCCTCTCTAGAAAAAAGGTGTAGG
GAATATCAGACAAGAATCTCAGTAACAGTTGTTTGACATTCATTACAATCTGAAATGTTGACCCAG
GGCATCAGAAATGTGCTGCTCCAAGGCAGACAAGTTATACCATTGGAAATGGATACCTGCTATGG
CCACGGTGGCACTGGCTGCTGTCCCATCAGTA

| | |
|---|---|
| Sequence 772 | cMhvSB027g06a2 |

CCCTTTCGAGCGGCCGCCCGGGCAGGTACCTATGACCATCTTACATTATTTTTATGGGTGGGGGGC
ATTGACTGTGGAATGTGGGCAGTAACTTGCACAGTCAGTAACCGTTTGAGTAACTTCTTGTTGGCA
TCCCCATTCTGGCACTCCTCCTCTAGGTCTCCACCTCACACGCTGGTTTGTGGGCGGAGGGGCAGG
TTGGTGCGTGGGGTGTCCGGGCACTGGCTGTGCATGCCTTCTTCCTCTTCTGTCTCTTGGCCACCTT
TTCCAAAAAGTCACCAGTGACCAATTCTCCCAGTGTTTCTTTGGGACTCAATGCCTTGGGCTTGGC
ATTGGGTAAAGCCAACTGGCCAGTTTCATTCTGACGAGCTCTATAGTAGTCCGGTGTGGACCTCTG
CCCTCCCTGCTCTGCGGAAGCTTCCTCAG

| | |
|---|---|
| Sequence 773 | cMhvSB027h07a2 |

ACGCGGGAGGCTGTAGGAGAACAATGAAAGGGAGGATGAAGAGATGGGTAAGTGAGCCATACTC
AAGGGCACATGGTGTTTCAAAAACACCTCCCACTATTTGGCTTTTATCCTTGAAAGAGAGCTCATA
AGAAAGTTTCACCAGGCCCACTGAAGTAGAAAAGCATAATAATATACTTGGTGAGTAATCTAACT
TCTTTTTCTCCAAAGGCTAGTAATCACCTATAAATTAAAATAAAGCACTTAAGTTTTATAGCAAAA
AACAAACAAACTGGCGATTTTCACTAAAACCAAAAAAAAAAAA

| | |
|---|---|
| Sequence 774 | cMhvSB029a10a2 |

CCCTTTGCCGCCCGGGCAGGTACCATCCCTCTCCTGAGCTAGACAATTATCCTTTGGGTAGTGTGA
AACTGAGTGTCTCTGGACTCAGGACAGTGTGCAAACAGTGGGGTTAAGACATAGGTTCATGTATTT
AATTGAAGACTCCCTGCTTTCTCTTTCGGACTTGTCTCCCACACAATAGCAGCCAGATGTTTATCTC
TAAGCAGCAACTGGAATTTTCTCTGTGGTATCTGACTAGTCTAAGAGGAATAAAAGACCAAAGAA
GCTGGCATTGTGGCTCCCCAAGGAAATGGCCTAATCCATTATTCTAACAGTGGATGAACCCCTTTC
GTGTACCTCGGCCGCGACCACGCTAAGGG

| | |
|---|---|
| Sequence 775 | cMhvSB029b03a2 |

GGTACCTGTTACCTGAGTCAACAGATCCAGATGAGAGGTGTAGGCAGGAGGGTCATCTCTGTGCA
TTTAGGAAAAGCAGCACTGATGCTAGTAGAGCATCCAGTTCCCCAACATGATCACCCCTGAAGCCT
TAATTCCCAAATCCTTCCAAGCCTTATCTGTAGGGGCTTAATGAGGACAGAAAGGAAGAAACAGT
CACTCTGGCACAACAGGACAATATATTCAGATTAAATCTGAAAATGGTGGAGGCCTGCTGCCCAT
GAATTCTGAGCCTCTCCAACCCTGGTCCCATAATGAAACTAGTAGTAGGGTCTTCCAAATGGCATT
AGACAAGGGTTCCATCTGTGTAAGGACCACTGGGAGTTAGACTGGACCCAGGATGGTATGCCATG
TGCAGCCATGTCAACCCCCAATTTGC

| | |
|---|---|
| Sequence 776 | cMhvSB029c11a2 |

ACGCGGGGATTTAAAAAAAAAACAACACCTATATAAGGGAGTGATCTACCATAATAAGATAACAG
AAACAACAAATGAAAATATTAGTACCCTCTCCCTGAAAATTTGAGTAATANATTATTCTGAAGTAC
TGTACTTCATTAAAAAAAAAAAAAANATNACNTTCCTTGTAAAATTACCGTTGTTNTNTGTCCCNC
CAAAAAAAAAAAAAA

| | |
|---|---|
| Sequence 777 | cMhvSB029f01a2 |

CCCTTTCGAGCGGCCGCCCGGGCAGGTACGCCTCACCCAACTCAACTCTTCACATAGCTCAAGTCT
TGGCATAAATGATATTTCTCAAGAGATACATTTTCTGACCACTTTATCCTTGTCTTTCCTTCATAATT
AATCCATAACATTATGCTTGTTAGCTTCCTTCATGGTATATATCATAGATTGTCATCATATATTAAT
ATGTTTGTCTATAGACTGTCTCTCATATTATATTCTACCAATATGAGTGCAGCATCCATATACCATA
GACCTAGCATGGTCTTAGATAACTAAGATCAAATAAATACAAAAGTTCAAGGGCAAATAATAACG
ATAATAATTAGGATTCTCAAAAGCATAAAGGTATGTTTTTAAAACTCTCAGGTATTAATAAAAATC
AATACCCAAAATTCTA

| | |
|---|---|
| Sequence 778 | cMhvSB030b11a2 |

ACGCGGGGTCACCTGCTGTGCTCTTGCTTGCACAGTGTCCTGGAGCTGGACCTGGCTCTGGGTTTC
CAGGAAGCAGTTTGACTAAAGGCAGCAAGCTGCTTCCTCTGCTGCCTGAGATACCAGATTCCCAAT
GGCGAAGATTGAGAAAAACGCTCCCACGATGGAAAAAAGCCAGAACTGTTTAACATCATGGAAGT
AGATGGAGTCCCTACGTTGATATTATCAAAAGAATGGTGGGAAAAAGTATGTAATTTCAAGCCAA
GCCTGATGATCTTATTCTGGCAACTTACCCAAAGTCAGGTACC

| | |
|---|---|
| Sequence 779 | cMhvSB030f03a2 |

NGTACTTATAGGCAATAAGGCGAGTCTAAGACCTAAACTAGATAATTTGAGAACAGGGAAAAAAN
ATTCCATTTCGATTCCTGAAGGTTACCCCCATACCTATTATAACAGAATAAAATAAAATAATTCNA
AACTGCACAACCTCTAACTTATCAAATCCTATATATGCCTCATTTTCTCAAATGACTCCTAATTTGT
GTAAAGAAAAAGGCAAAAAGAGAAAGGACAGAANTATGTCAAGGTGGGCTAAAGCTATGAATAC
CCTTTTATGTAAACTAAGAAAAAAATANATACACACGCATTTTTTAAAAGGGAACTTTTTGAAACC
TTGAGCCGCAAAGAGGAAAAATTCCTGGCTAAATTGCACCACTCAAAGACAACTAGACTTACGGT
CATAAATTTCTTCTCCAACCCATTTCTTTCAGGATTCTTACAGATCCATAGCATTTTGCAAGCTGAC
ATAGGACCCTTTCA

| | |
|---|---|
| Sequence 780 | cMhvSB038a01a2 |

CCCTTTCGAGCGGCCGCCCGGGCAGGTACGCGGGTAAATCGAATTAAACTAAATTAAACATTTTTC
TTTCATTAGTAATATTAAAACACTTAAAGCTACATTGAGTGATAGCAAATTAGTAAAGCCTATTAA
GTCTTCTATGTAAAGTATGATTCAGAAATATATATTTTATATATATATGCATGATCTCGGCTCACCG
CAACCTCCGCCTCCCAGGTTCAAGCAGTTCTCCTGACTCAGCCTCCCTAGTAGCTGGGATTACAGG
CATGTGCCACTACGCCCGGCTAATTTTGTATTTTTAGTAGAGACNGGGTTTCTCCATGTTGTTCAGG
CTGGTC

| | |
|---|---|
| Sequence 781 | cMhvSB038b08a2 |

NCGCGGGAGGCCATCTCGCTATAGGAAAGGAAAGTGGAACAGCATTCATCCTCAACATTTTTACN
AAGACAAAATGAANACTGGAGTANAAGACTGATCAGTGCAGGTGTAGCATAAAAGTGTAATCCTG
GAAGATGTGGTGTGAGAAGGTAGCACAAGTGAAGCAGAGATACAGGAGATANGGAAGGGAAGCT
GGAANCAGANGTCACTGGAGGGAGAGGGAGATNGACACATTCAGGGCTACNAAGCAAGTTCTAT
GTGATNNGCTCACCTCTCAATTGTGGNGACCCCTC

| | |
|---|---|
| Sequence 782 | cMhvSB038c01a2 |

CCCTTTCGAGCGGCCGCCCGGGCAGGTACCTCTTATTCCAGAGAAGTGGGGAGCAGAGAGGAAGA
TGGAGTGGAAAGGGGCGAGACAAGGCCCTCCTGAAATACCTCAACCCAAATCTTCAAGAAATCCC
CAAGTCCCCACAGTGCTTTTTGTGGATTTTTGTGGAAACCGGTAAAAGGGGCTGATTTGCTGGCCC
CAGTGGGTAGAAAACAGAGACTGTCAAGAGAACAGAAGAGAAGGCAGAAAGGGGATGGGGAAG
TGGGGTTCGCCATGTTCACGAGCTCCTGGAGCCACAGGGCCCCCCAGGAACAACAGAGCTGAGAC
TGGGTGGCCTTGTTTCTGGCCCAATTCCCTGGGACC

| | |
|---|---|
| Sequence 783 | cMhvSB038g08a2 |

CCCTTTCGAGCGGCCGCCCGGGCAGGTACGCGGGAATGATTTATTTGAGGGTTTGGTACATCTTAT
ACAACCGTGAATACAATTTGCATCTAATAATGTGACTTCAGTAGTATCATGATTTTTGTCCAAACCT
TCTCAGTCTGGGAAACATTTAAAGAGAATAATGACCTTAGAGAAGAGCTGGATTTCTTTTAAGACT
TNTATTCAGATCAGGACACAATCACGTTCAAAATTGACATNANCATGTAACATGGATTTCAGTGAA
GAAAAGTACTTNAGAATCAAATTTTAGAAGAGTGTTTTAAGGTTTAGTGGCCCTAATCAAAAGGA
NGTCAAAAANCTNTTTTTTTGGTTAATCCATTAGGGNGGNGNGGANCCACCNGGGGTTTTGGCCTC
TTNGGTTTTNNTTTTGAAATTTGGCCCAGGGGGCTACCTTTGGTCCANTTTTTTNGGGGGAAGGGA
AATNANATTGGGNCNCNAAAAACTTTTGGGGGNAAAAANTTANAANAAATTTTTTTNNTTTNNCTT
TTGGNAAAGNCCTTTNCCNGGCCNTTTTTTTAAAAAAAAAATTGGCCTTTCCGATTTTTTTTNAAAT
TTAAAAATTTNGGNTTTTTTTTTTGGAAATTTNGNTTTNAAAACTTGGGGGTTCTTTTNCCCCCCTTT
TTTTTTT

| | |
|---|---|
| Sequence 784 | cMhvSB049c05a2 |

AGGTACGCGGGGGACCTGCTGTGCTCTTGCTTGCACAGTGTCCTGGGAGCTGGACCTGGCTCTGGG
TTTCCAGGAAGCAGTTTGACTAAAGGCAGCAAGCTGCTTCCTCTGCTGCCTGAAATACCAGATTCC
CAATGGCGAAGATTGAGAAAAACGCTCCCACGATGGAAAAAAAGCCAGAACTGTTTAACATCATG
GAAGTAGATGGAGTCCCTACGTTGATATTATCAAAAGAATGGTGGGAAAAAGTCTGTAATTTCCA
AGCCAAGCCTGATGATCTTATTCTGGCAACTTACCCAAAGTCAGGTACCTGCCCG

| | |
|---|---|
| Sequence 785 | cMhvSB049c11a2 |

TAGCTGTTTCCTGTGATGGTAAAAGGACCGTCCACCGCGGTGGCGGNCGCCCGGGCAGGTACGCG
GGAATGATTTATTTGAGGGTTTGGTACATCTTATACAACCGTGAATACAATTTGCATCTAATAATG
TGACTTCAGTAGTATCATGATTTTTGTCCAAACCTTCTCAGTCTGGGAAACATTTAAAGAGAATAA
TGACCTTAGAGAAGAGCTGGATTTCTTTTAAGACTTCTATTCAGATCAGGACACAATCACGTTCAA
AATTGACATAGCATGTAACATGGATTTCAGTGAAGAAAAGTACTTCAGAATCAAATTTTAGAAGA
GTGTTTTAGGGTTTAGTGGCCTAATCAAAGGGAGTCCAGAAGCTATTTTTGGATAATACATAGGAG
GTAG

| | |
|---|---|
| Sequence 786 | cMhvSB063b04a2 |

GCGCGTCNTGGCGGCNTCCGCCAACTGATTGGGCGAACCGTCCAGGTCCAGCTTGCCGTGCANCA
GGCTGAGACTGGCCGCATTCGCGCCGCCGCCGCCCAGGCTGTCGAACANATTGCCCGACAGGCCG
GCCGAGAAGCCGCGGATCGTGTAATTGCTGCTGGTGGCGCCGTTTGCCTCGTTGTCGAAACGCTTG
TCGTCATAATTGAGTTGCAGATACAGATTGCGCAGGCGCGAGCGCAGCAGCGGGTAGCTGGCGTC
GACGCCCAGCGTGTTCGAACTGCCCTTGGCGTGCAAGGCGGCAAATTCNTCGGCC

| | |
|---|---|
| Sequence 787 | cMhvSB063b12a2 |

ACACGTGTGCACGCACATGCACATGAACACAGGAATGTTCTCTCTCACATACAAAGTTGAGAGAG
TAAAGCCAAATGTGTTTTCTCTTTTATTTTGGCAGCATTTTAGATATTTAAATTTTTAGCTATCTTAA
GTTAATCTAAAAATTTGAATAGATGAAATGAGCATATCCCATCCTTTATGAAGAGAATAATCAAAA
TACTGGAGTATGCTCCTCTCTAGAAAAAAGGTGTAGGGAATATCAGACAAGAATCTCAGTAACAG
TTGTTTGACATTCATTACAATCTGAAATGTTGACCCAGGGCATCAGAAATGTGCTGCTCCAAGGCA
GACAAGTTATACCATTGGAAATGGATACCTGCTATGGCCACGGTGGCACTGGCTGCTGTCCCATCA
GTACCTNGGC

| | |
|---|---|
| Sequence 788 | cMhvSB063d06a2 |

AATTGGAGCTCCCCGCGGTGGCGGCCGAGGTACGCGGGGGGCCGGAGCCGGGCCGGGCAGCTAG
CAGGGCGCTTCGGTCTTAGGTATGTCTTTATCAGCAGCATAAAAACGGACTAATACAAGTACACAA
GAATACAAAGAAAAGAACAGCAGACACTGGGGCCCGCTTGAGGGTAGAGGATGGAAGGAGGATG
TGGATCAAAAGCCTACTTATCAGGTATTACGCTTATTACCTGGGTATTGAAATAATCTGTATACTG
AACCCCTGCAACACGCAATTTACCCATATAACAAACCTGCAGACGTACCTGCCCGGGCGGC

| | |
|---|---|
| Sequence 789 | cMhvSB065d05a2 |

GATTGGAGCTCCCCGCGGTGGCCGGCCGNCNNGACNNGTACTNNATTCACGCCTGCACNNGTTTA
AAGCCTGTNTTATNTATANNTGTCCNGTCATGGGGGGNNCTTTGACTCTTATGATNCANTGNNGAA
ACNTGGATTNNNTNTCCNNTNNNCTNNTGNTGGGGANATGCTTTCTNNNAGTGACGGCAATGGAA
ATATCAAGCAACCAAGGGAAATCTGAAGATCCCAGAGAGCCCAGCAAGCAGCAACATCCTCGAGT
TAGGCAAGCAAGGGCCCGGAGCTGGCCAGACCATGGGCTGGAATGCAGTGGGGGCCGGTCAGAG
GGGCTTCTTCTGGGGTCCTGACTGTGGTTTCTGCCAGAGGTGGAGCAAGTTGGAACTGGATGTTGA
GTGAAGTTTCAAAGAACTTAAAAGTCAAATGGGGAACAATAATCAAAGGCTTCCATT

| | |
|---|---|
| Sequence 790 | cMhvSB065e04a2 |

CGAGGTACGCGGGACCTGCTGTGCTCTTGCTTGCACAGTGTCCTGGAGCTGGACCTGGCTCTGGGT
TTCCAGGAAGCAGTTTGACTAAAGGCAGCAAGCTGCTTCCTCTGCTGCCTGAGATACCAGATTCCC
AATGGCGAAGATTGAGAAAAACGCTCCCACGATGGAAAAAAAGCCAGAACTGTTTAACATCATGG
AAGTAGATGGAGTCCCTACGTTGATATTATCAAAAGAATGGTGGGGAAAAGTATGTAATTTCCAA
GCCAAGCCTGATGATCTTATTCTGGCAACTTACCCAAAGTCAGGTACCTGCCCG

| | |
|---|---|
| Sequence 791 | cMhvSB065g08a2 |

AGGTACATGGTCTTTGAACTCTCGTGTCGAAAGAGTTGAACACAACTAAACTTTAATGTGAAAAGG
TCTCAAGTAGTTAATCAGAAATGAGAGGCGCACATAGCATTTTATACTGTTTTCGATTTGCTGACA
CAACATCATTCTGTGCTCTCTAGTGAGCAAGAGTAATCCTCAATAGCATTAAGACGAAAGGCTGAA
CACAAAACCGCAGGCAAGTCAAGTAGTGATTTTATTCTTTTTGTCATTTTTCTTTCAAGTGGAAGAT
CCCTAACACTCTCTGCTCCTGACAATGTTTATAAACAGAACTCTGAGAAGCATCTGAATGTAAAAA
A

| | |
|---|---|
| Sequence 792 | cMhvSB071b04a2 |

AATTGGAGCTCCCCGCGGTGGCGGCCGCCCGGGCAGGTACGCGGGTATTAAATTTCCAATGTGAT
GTGGCTTCTGTTTGGATAGAGATGGAGCTGGTCTATGTTTCTTTACTCTGTGTTCATAGTATCAAAG
TAAGCTTTGTATCTGTTTTTCTGTAATGATGACATTTACACTTGGTTGCATTAATATGAAGTAACAT
GGATTGCGTGTGTTAGTAGGTTCTTTTTAATTACTGTGTAAAAATAATATGTAATTGAAACAAAAA
GCATTGTTTCCAATCCTAATTTTTTTTCCTCAAGTCCATCCTGTCAAGCTGCAAGCGTGAAAGTTAT
TTTCTGGTGGTGTGATTAGATTGGGGCTGAACCCTCCAGCTG

| | |
|---|---|
| Sequence 793 | cMhvSB071d02a2 |

ACCTGACTTTGGGTAAGTTGCCAGAATAAGATCATCAGGCTTGGCTTGGAAATTACATACTTTTTC
CCACCATTCTTTTGATAATATCAACGTAGGGACTCCATCTACTTCCATGATGTTAAACAGTTCTGGC
TTTTTTTCCATCGTGGGAGCCGTTTTTCTCAATCTTCGCCATTGGGAATCTGGTATCTCAGGCAGCA
GAGGAAGCAGCTTGCTGCCTTTAGTCAAACTGCTTCCTGGAAACCCAGAGCCAGGTCCAGCTCCAG
GACACTGTGCAAGCAAGAGCACAGCAGGTCCC

| | |
|---|---|
| Sequence 794 | cMhvSB071e04a2 |

AGGTACCTGACTTTGGGTAAGTTGCCAGAATAAGATCATCAGGCTTGGCTTGGAAATTACATACTT
TTTCCCACCATTCTTTTGATAATATCAACGTAGGGACTCCATCTACTTCCATGATGTTAAACAGTTC
TGGCTTTTTTTCCATCGTGGGAGCGTTTTTCTCAATCTTCGCCATTGGGAATCTGGTATCTCAGGCA
GCAGAGGAAGCAGCTTGCTGCCTTTAGTCAAACTGCTTCCTGGAAACCCAGAGCCAGGTCCAGCTC
CAGGACACTGTGCAAGCAAGAGCACAGCAGGTCCCCGCGTACCTGCCCGGGCGGCCGCTCGGCTC
TAGAACTAGTGGATCCCC

| | |
|---|---|
| Sequence 795 | cMhvSB073b05a2 |

GATTGGAGCTCCCCGCGGTGGCGGCCGCCCGGGCAGGTACACAAAACAGAGATGCACAACTACCC
TACCACCTGGGCAAGAAACGGGCTGCCACCTGGCATCTAGAAGCAGCCCTGTGACCCCAACCGCT
ATACTACACCCTTCTTCACCTCCACTGCTAAGTTCATAATCCTTTAATCTATCATCCCCACGTGTTG
AAGGCAGCTCCCTTCATAATTCTTACATTCAATTCCAAAATTCTGAAACT

| | |
|---|---|
| Sequence 796 | cMhvSB073g06a2 |

NGATTGGAGCTCCCCGCGGTGGCGGCCGAACGCGCGGCCCTGGAGTTGCGTCGCGATGAAGCCGT
ACGCGCGCTGCAGGACGAAGACAAGCGCTACCAGATCGTCAAGGACATCGCCGATGACCTCAAGG
TCGGCTACAACA

| | |
|---|---|
| Sequence 797 | cMhvSB075b08a2 |

CTGATTGGAGCTCCCCGCGGTGGCGGCCGAGGTACTTCTAGAATCCACAGCTCTGGGAGGGCTACC
TTAAATTAACACTGGCAGTTCTTTGCAATTAGGGTGCCATAAAAGCAGCACAGTTGACTCCAAAAT
GGACTGAGTTTTGGAAAGATGTCTGCCAGCAAAATCATATAGACTTTCTTGCTGAAGGGATGAAA
AATTAATAATGCCTTGAAGTATATTAATATAAAAATATGTGACCAAGCAGTGTAATTAATTCCCCT
TTTTCCTCAAAATGTAGCCTTTTTTTTTTGANATGGAGTTTCACTCTGTCACCCACGCTGGAGNGCA
GNGGNGCGATCTNAGCTCACTGCAACCTCAACCTCCTGGGTTCAAGCAATTCTCCTGCCTCAGCCT
CCCAAGTAGCTGGGACTACAGGTGTGTGCCCCATTCCCAGCTAATTNGTNGGATTTTTTTT

| | |
|---|---|
| Sequence 798 | cMhvSB075d02a2 |

AGCGCATGTAGTCGTAGCGGTCGGCCGCCAGGCTGCCGCTCTGCTCCTTGTTGCGCACGATGACCG
GGCGCAGGAAGATCATCAGGTTGGTTTTCTTGCGCTCGCGCGTCTGGTACTTGAACAGGTTGCCGA
TCAGGGGAATGTCGCCCAGGCCGCGCACTTTCTCCGCGTTGTCGCCCGTGGTGTCCTCGATCAGGC
CACCCAACACGATGATCTGACCATCGTCGGCCAGCACATTGTTTTCGATCACGCGGTTGTTGATGG
TGATGCCGCTGACGGCCGACGCGGTGGATTTGTCCACGCTCGACGTCTCGTGATAGATACCCAGCT
TGATCGTGCCGCCCTCGGAAATCTGCGGGCGCACCTTCAGGGTCAGGCCCACTTCCTTGCGGTCGA
TGGTCTGGAACGGGTTCGTATTGGT

| | |
|---|---|
| Sequence 799 | cMhvSB075e06a2 |

CGAGGTACGCGGGTCTTCTCTCCTCCTTATGCCTTTTCTTCTTCCTCCTCACCCTCATGGCTCCAGGT
CCATGCCCAGGGAGCATGTTAGCATGTTGTCAGGTCTCAAAGTATCTGAAAAGATTGTCTTCTCTG
TGGCCAGGCTGCTTAGAGGCAGCCTGATATAAACTGTAAAAAGGGGGAGAGTGTTTCTCTGTGTCC
TCTGCATCCACTCTTCATGCATTTGCTCCAAACCAAATCTGCTCTTAGGAAGGGATCAGACGAACC
TGTTTAGAGTGAGGTAGCAATGATAGGTTAGCAGTGGGTAAACCACATAAATGAAACTTTAAATG
AGGAATTCCACCTTGTTAAAGAAGTAAGGTGGGCCAGGCACAGTGGCTCACGCCTGTAATTCCAG
CACTTTGGGGGGCCAAGGCA

| | |
|---|---|
| Sequence 800 | cMhvSB075f02a2 |

TGATCCCTNANGCTCCAGCCTTCGGGAAGATATGTCTACAATGACCTTTGGCCACTGACAAAGAGG
AAGTTATCTGGAAGTTTGCAAACCTCTGTTCAACTCTCTATCCACCCCTTGGAAGGACCTTTTCAGA
GGAAGANAACAGAGTTTGTTTTTCAAATCATTTTCACCATATCTAAAACTANCCACTCNGCTTGGT
GATAGGACATCCCTATGAAACACACATG

| | |
|---|---|
| Sequence 801 | cMhvSB075h08a2 |

CGTGAGCCTCGCGGATGTGGCCAGGGAGCCGTACATTTTCCTCACCGTCGACGAGGCCGAACAAA
GCGCCATGCGCTACTGGGAACAGGCCGGGCAAACGCCCAAGGTGCGGCTGCGCACCAGTTCGGTG
GAGGCGGTGCGCAGCATGGTCGCCAATGGCAGCGGCGTGGCAATTCTGTCGGACCTGGTGCATCG
CCCGTGGTCGCTGGAAGGCAAGCGCATCGAAACCGTGAGCGTCACCGACAAGGTCACGCCCATGA
GTGTCGGCCTGGCCTGGCACCGCGAGCGCGACTTCACCCCGGCGATGCAGGCGTTTCGTGATTACT
TCCACGATGCATTCCTGGCGCCGCANCAGTTGTCGGCCCGGCGTTAAAGCCGGGATTGCAGGATCG
CCGCCAGCCAATCCATGAACACCCGCACCCGCTGCGGCAAATGCCGTTG

| | |
|---|---|
| Sequence 802 | cMhvSB079b02a2 |

TTGGAGCTCCACCGCGGTGGCCGAGCGGCCGCCCGGGCAGGTACTGGGATGAGAAGCTCAAGTCC
CTGTCCTCAAAAATTTACTTTCTAGCATTGATGAATAATCAGTCTTCACTATTTATGATTAAAAAAA
CTTTGTTCATCATATGCTTTATTTAAAGATTGATAATCTGTTCTCCCATTACCTGGCCACTTGCTCTT
TGCTCTCCTAATTACTTCTTAGGACCTTTAGTAGCTTTCTTGTTTTCTGAGTATGGACGTTTTCCCTC
AAGTAAGACACTACTAGTCGCTGGGTGCGGTGGCTCACGCCTGTAATCCCAGCACTTTGGGAGGCC
AAGGCGGGTGGATCACTTGAGGTCAGGAGTTTGAG

| | |
|---|---|
| Sequence 803 | cMhvSB079e11a2 |

ACTACCAGGATGGCCGCACGGGCAACGCCAAGCTGGGCGACATGGTGGCGCTGGGCGGCGGCAA
GTTCCTCGTCATCGAGCAGGGCGCCGCGCCGTCGGGCAAGGTCTTCAACAAGCTGATGCTGGTCGA
ACTGAAGGGCGCCACGGACATTGCGGCTGCCGCTTTCAATGCGACGACGTCCGACCTGGAAAAAA
GCAGCATGGGCGGC

| | |
|---|---|
| Sequence 804 | cMhvSB080e06a2 |

CCGGGCAGGTACTGGGATGAGAAGCTCAAGTCCCTGTCCTCAAAAATTTACTTTCTAGCATTGATG
AATAATCAGTCTTCACTATTTATGATTAAAAAAACTTTGTTCATCATATGCTTTATTTAAAGATTGA
TAATCTGTTCTCCCATTACCTGGCCACTTGCTCTTTGCTCTCCTAATTACTTCTTAGGACCTTTAGTA
GCTTTCTTGTTTTCTGAGTATGGACGTTTTCCCTCAAGTAAGACACTACTAGTCGCTGGGTGCGGTG
GCTCACGCCTGTAATCCCAGCACTTTGGGAGGCCAAGGCGGGTGGATCACTTGAGGTCAGGAGTTT
GAGACCAGCCTGGCCAACACG

| | |
|---|---|
| Sequence 805 | cMhvSB082b09a2 |

AGGTACAATGTGGGACTTTGGTGGAACTGCCTGGGAGAACTTCATAATTACTACCCTGTATGTCAT
GCCCCTTGCAGGTAAAACAGAAGTGGCAGAGCAGAGGTCAAAGGCACAGATCAGCAAAGGGAAT
CCTACTGGATCCTGAGACTAGCCTGGAAGGGGTGTCATTTGTCACTGGGAATAGAGGTGCACGGC
CTGGTGGACCCTCCGAGAGAGCTTAAGATTCATTTTTAAAACAGAGGATTTAAAAGACACAATAG
GCATTGGAATCGGGTAGTAAGAAGAGAAAACCAGAGCCCCAAGTGAGGAAGTGGGTGATCTGTCC
TCACACAGTTGGTGGGGGAGCTGGGCCTCCCCACTGACTGGACTCTCAGGTCCTTAGGAGGTGCTC
TGTCCTGCACACCCCAAATGACCACCTAAATTCAGGCCTGAAGCAGTAAGAAGTACCTGCCCGGG
CGGCCGCTCG

| | |
|---|---|
| Sequence 806 | cMhvSB090b03a2 |

GGGCGAATTGGAGCTCCCCGCGGTGGCGGCCGAGGTACTCATTGGAGGATCAGCTCACCTGCTTTG
CTCTCGATGTAGCCTAGCTGGGTTTAGAGCCTTCCCTTGAATGAAGAACCCTCCCCAGCTGGAAGG
GGATGCTCTTGAAAGCTCAGCTGACAACACACATGGGCATCAAGTCATTGGCCACATTCATGCCTC
AAGTGTCCTAAAACCGAATATGATCAAAAGAAAACTGCTGTTCAGCAAGTGGAGACTGGCATGCA
GATTCCCTGGCCTGCAAGCCTAGTGTAAAAAGATACCAAATACTGCTGGAAGAATGAAAAGGATG
AAGGGATGTCATCAAAGTAGTTTTTTCACTTGATGGAAAAGACTAAAACAGCAAAGCAAGTTCAA
GATCAAACACAACACCACAGGGATCCTTTGATGAGAAGTGAACTTAAGACCATGAAATGCTGTT

| | |
|---|---|
| Sequence 807 | cMhvSB090c01a2 |

ATGTACANNTNNTGAANNNNCCNNCCTGCNAGANNTNAANATANNACNTATAAATNCCTTNGACC
TCCNGGGGGGGCCCATNTCCCNCNTNCTGNACCNATTCACTGANGGNAAATTGCCCNCTCGNGTA
ATNATGGTCATATCTNTTGCCGACCTTCTCACACCACATCTTCCAGGATTACACTTTTATGCTACAC
CTGCACTGATCAGTCTTCTACTCCAGTCTTCATTTTGTCTTCGTAAAAATGTTGAGGATGAATGCTG
TTCCACTTTCCTTTCCTATAGCGAGATGGCCTGTCCCGCGTACCTGCCCG

| | |
|---|---|
| Sequence 808 | cMhvSB091b10a2 |

NATTGGAGCTCCCCGCGGTGGCGGCCGAGGTACCTTATTTACACCCATGTGCAGGGCAAGGCAAG
CTAGATATTTGCTGTTGTTATTGGGGGGCAAGCTCAAGTTCAGAAATGGGAAGAAAGATGCAAGG
GGAAGGGCCATGTATCTATTGTGCAGGGAGGAATGGCTGCCAATTTTCCAGGCATGGTCTCCCATT
TCCCACCCAAAGGAGGAAGCCAACCTATTCAGAAGCCAGGTACCTGCCCG

| | |
|---|---|
| Sequence 809 | cMhvSB091c11a2 |

TNNTTNNNNGTGAAAACCCNNAGTNTCANTGANNATGNTTTCTNGCNGANNANNNCCNTCTATNN
CTNCNNGNGNNNNNNCTCCTNGGTAACGCNCNANTNCACNAGNNTNTATCTCCTACTGGCTGNAA
NACTCTCCNNACTCNCCCNCCTNCCT

| | |
|---|---|
| Sequence 810 | cMhvSB092a12a2 |

ACAGTGTGGCCTAAAACAGAAGAATGTTTAACTGCATGAAGGCAGGGTGGTTTGTATTGCTGGGC
TTGGTGTATATTTCTTTGCTATCTAGTTTAATATATTGAGCTTTACATCTGTGCCAGCCTTGCATGTC
CATATACCTTTGGCAGGCATTTCTAGTCAGGTGGCATGGGGCAAGGGGTGTGCTACGTTTTAAGTC
CCTCATTTCTCCAGCCTGTCCAGGTAGTGTCTACGTCTCCAACTCACTCAGGAAGGCAGGAGACTT
CCAGATTCACTCCACTGGTATCAAGAGTTAGGTTCTGGTGAGAGAGCTGGCAGAAGCTTCAGAGG
ACCTTGCGTCTTAACCTCCTCTTTTTTTTCCTGTCCTTAACAGCAAGTTGTTGCCTCTAATTTTCAAA
AAATCGCAACACATTTCCAGGAGACCTGAAATGCGGTGGACTGCTTCAACATTANATTNTTTTTGG
CAGACANGGATAGTATTTAGTGTAACGTCACCTATATGCTTATCAAATANGGGTAAGGGGAGTCA
TAATTATT

| | |
|---|---|
| Sequence 811 | cMhvSB092h02a2 |

GGAGCTCCACCCGCGGTGGCGGCCGCCGGGCAGGTACGCGGGATGTCCCTGAAGTCCTCCAGGCC
CACACCTCCACCCGCCTTCTGTCCTGTATCTGCGGAAATATTTATTTTCTGTAATGAACTTTCTTGG
GGCTCCAGACACCCTCTCAGCCTCTTCCCACACAGAACTTTGCCTACACATTCCTACTACCCCTGGA
ATTCTAACTCAGATGTGGGTAGCAGCTTCCTCAAAGAGAAACTTTTCCCAGCTGGGTGCTGTGGCT
CACACCTGTAATCCCAGCCCTTTGGGAGGCTGGAGTGGGCAGATCGCTTGAGCCCAGGAGTTTGA
GATCAGCCTGGGCAACATGGTGAAACTCCATCTCTGTGAAAAATACAAAAATTAGCCAGGTGTGG
TGGTGCGCGCCTGTAATCCCAGCTACTAGGGAGGCTGAGGTGGGAGGATTGCTTGAGCCCAGGAG
GTTGAGGCTGCAATGGGCTGCG

| | |
|---|---|
| Sequence 812 | cMhvSB093a10a2 |

GGAGCTCCCCGCGGTGGCGGCCGAGGTACGCGGGGACAGGCCATCTCGCTATAGGAAAGGAAAGT
GGAACAGCATTCATCCTCAACATTTTTACGAAGACAAAATGAAGACTGGAGTAGAAGACTGATCA
GTGCAGGTGTAGCATAAAAGTGTAATCCTGGAAGATGTGGTGTGAGAAGGTAGCACAAGTGAAGC
AGAGATACAGGAGATAGGGAAGGGAAGCTGGAAGCAGAGGTCACTGGAGGGAGAGGGAGATGG
ACACATTCAGGGCTACAAAGCAAGTTCTATGTGATTTGCTCACCTCTCAATTGTGGGACCCCTCAA
AATGTGTACCTGCCCG

| | |
|---|---|
| Sequence 813 | cMhvSB093b08a2 |

AAAATGGCCAAATAANGAGGGAAAGGTAATAGCTTTGCTGTCGTGACTACCACNATGAAAGGATC
TGGCTCANGCCCTCAAGGAGGGCATTCTTCCTTGCGTAGTTATTGAGAATATGGCTTTCTAGTTAA
AGTCTGGCTCTGCCCCTTAAGTCNGCAGGGTGAACACACCAGGCAAAAGAGGTGTGTGTGAANGC
CCACAAGTAAGGGGAGACACACCCTTTCCC

| | |
|---|---|
| Sequence 814 | cMhvSB093e10a2 |

GGGCGAAGCCGCCATGGTCGACCACCTGCACAAAGTAATACAAATCGTTCAGATCCTGCATGCCG
CCTCCTTGATCGTTCTATTTTTGGAACGCTGATGGCGAATTTTACCGTCTACCGCCTCTATCGTTGC
AAGAGTATTCTGACTCCATCGTAATGCACACCCTACAGGAGATCGAGATGAACACANTNNCAGGT
ATCTACAGCGCACCCNGCCAGCACTGGGTNGGCGACGGTTTCCCCGTGCGCTCGATGTTTTCGTAC
ACCGGCCATGGCAAGCAGCTGAGCCCCTTCC

| | |
|---|---|
| Sequence 815 | cMhvSB093g08a2 |

GCTCCCCGCGGTGGCGGCCGCCCGGGCAGGTACACATACATAAAAGAAAATGGCCAAATAAAAA
GGGAAAGGTAATAGCTTTGCTGTCGTGACTACCACGATGAAAGGATCTGGCTCAAGCCCTCAAGG
AGGGCATTCTTCCTTGCGTAGTTATTGAGAATATGGCTTTCTAGTTAAAGTCTGGCTCTGCCCCTTA
AGTCGGCAGGGTGAANACNCNANGCAAAANGAAGTGTGTGTGAAAGCCCANAAATAAAGGGGGA
GACACACCCTTTCACCCTTTCAAGCAAGGCCTTGATCCTTGCTCCCCCACAAAAGNTTGTNACCTG
GTTCTGTCCTCTAAAACATTCCANGAAGGTAAAGGCTGCAAGAAGAANCCTGGTTCTTTGAGCTTC
CAAAAAAAAAAGT

| | |
|---|---|
| Sequence 816 | cMhvSB094a08a2 |

TTGGAGCTCNCCGCGGTGGCGGCCGAAATACCGATATTGACTTCCGTAATGGTCGCGGCCGCCGGC
GTGTAGTGCAATTCCAGCGGCAAGGTGTGGGTGGCCGTGGCCATGCCTTGCNACACGGATACATA
CGGCCCCAAGCAGATTGCCCGTCAACTTGATCGGCTGCATCACGGGACGGGCGGCCTTGTTCGGCG
AGCTGAACTTCGCCTGCGTCACCTTCAAACCCTTGCACACGAGACTCANTTCCACCGTCGTCACGG
NGGCCAGGGCGCTGTCGCCGATATTCGTGGCGCTGACATTGCCGCCCAGGCTCAACGCCCCATCCT
TGGTTTGCTTGCTGACATCGACGCATTCCGTCCTGACGATGCTGCCCTGCGTGTAATGCGTCTGGTT
GACGGCGCCCGTGGCGGCCAGGTTGACGCCGGGCAGCCCCGTCACGGCGTACGACCAGGTATCGT
TGTACTGGCTTTGCGCCGTCGTGTAAACGGGGTAT

| | |
|---|---|
| Sequence 817 | cMhvSB094g05a2 |

TGGGCTTNTTCGTNGACCGTTTGCGCNCGGGCCTGAACCGCGACGCGCACCAGCTGCTGGGGGCC
GACCTGGNCATCAGNGCCGACCANCCCGTCNATGCNGNGTGGCGCGCCNAAGCGCACAAGCGCG
GTTTTATCCTGGCCGACACGGTGACGTTTCCCAGCATGGCGCAGGCGGGCGAGGGCGAGCAGTCG
CTGTCGCAGCTGGCGTCCCTCAAGGCCGTCTCGCCCGGCTACCCGCAGCGGGGCAAGCTGAAAAT
CACGACCAAACTGAACGAAGCGCAGGATGCCGTGGGCCANCCGACCAGCCAGGTACCGGCGCCC
GGCACCTTGTGGGTCGACGCGGCGATTTTGTCCANCCTGAACGCGAAACTGGGCGACACCTTGACC
TTGGGCGACAAGGCATTTACCGTCACGCAACTTGATCCCAGTGAGCCGGACCGGGGCCGCCTCGTT
CCTGAACTTCCCCC

| | |
|---|---|
| Sequence 818 | cMhvSB095b10a2 |

CCGCGGTGGCGGCCGANGTACCAACATGCTTTACCATGCTGCAAAATTTAGGATCCTGTGGCTGAA
ATATTTTGTAAGAAATGATGCATCCTGAATTTATCATTGAATTTCAAGTCTTGAAATAAGTAAATTC
ACATTTCCTTGTTTTGGCATAGAAGTGTTTAGCTGATTAAAGTTTTTGGCACTTGTTTTGCATTTCCT
CTGAGAGGGCACTAATGTATGAGAGAAGGTAAACCGAACCTTCTAAGGGAAAGGAAAGTTAAGG
AGGCAGGAAAAGCATCTATAGCTCTGTTTTCGGGATTTAAGAGTATAGGTTCTGGAGGCAGACTGC
TCAGCAGACTGGAGCCAGGTCCCAAGTCTGGCTTTGCCTGTCACTAGCTGTGTGAGCTCTGCCTTA
GTGAGTCTCAGCTTTCTCATCTGTCAAATGGAGGTGACGAGGGCTGTGGTGAGGA

| | |
|---|---|
| Sequence 819 | cMhvSB095c05a2 |

TGCTTGCTATCGCGCAACGTCTTGTCATGCTCGGAAGCCACATGCAACAGCCCGCCCTGCAAGGCC
GCTTCCATGGCGTCGAGCACCTTGAAAAACTGCTCCGTCGCTTCTTTCGAGACGGGCGCGAGCTCC
TGGCGCGGCTTGCGCAGTACAGGTGCAGGTCCAGGCTCGGCATGGGCCGCCGCGGCCTCTGGCGC
TGCCGCCTGCACGGGTGCCGGCNCGCTGGCCAACANGGCCGAAAATTGCTGCTGCAAGCTGTCGG
ACAAACTGNATTCAGGCAGGCGCGGCATGGCTTGCCACGCGCGTTTCATGGCATTCACNTTGAGGC
TGGCGGCGTCCTGCNCCTGCCATTCCGCCAGTGCGGTCTGGCGCGCGTCGAACACTGACT

| | |
|---|---|
| Sequence 820 | cMhvSB096g02a2 |

GTGTCCGGATGCTTCTACAGCACAGCGGAGCTCGATCGAAAGAGGGCAGTCGGGATCGTCCAGCC
TAACCATAACCGACTGGTCGGTGGCACGGTTCAGCTGAAGCTCCGCTGGCAGATCAGCATCTTGCT
GCTGGCCTTGGCCGATACGGCGTTGCATACCTTTCTGTTGGTACACCTGCAGGACGTGGTCGAGCA
GCTGGTGGCCATCGCTATCGGCCGGGAAGAGCTCCATGAAGCTCGTAGCGTCGACTTCGAACCGA
GGGTTGTCCTTGTTCTGTTCCTGCAGTCTGCGCTCGTAGTGGGCCTGCAGGCGGTCCAGTTCGCGCA
CTGCCCACGCGCAGGCGGAGGAAAGGCTAGTTTTGGGGCCAGTCACACCGCCTCCGGTATTCGGC
AAGTAGGTGCCAGGCGACTGGT

| | |
|---|---|
| Sequence 821 | cMhvSB097h05a2 |

CGAGGTACTTCCTGGCTTGTTGAGCGTGTCCTCACTGCTGGCCCTCTTGAGCCTGCTGAGTCGGGA
CTCAAAAGCCAAGGAAGTTGAAGACTTAGAACTCTTCATGCCGGAAGAGGCTGCAGGCAGAGGCC
GCACCCGGTCTGGGCCGTGGCCCCCTGCTCTGATGGATGGGTTCCAGGGCTTGGCTGCACTCCGCA
TGCTTGACTTCGTGGGTCTGTCTGCAAAAACTCTGCTTCTCCTGCTTCTCGGGAGCTGCCGACCTCA
ATCANCAAGTCANCCACTCTCCCGCGTACCTGCCCG

| | |
|---|---|
| Sequence 822 | cMhvSB101b10a2 |

GAGCTCCCCGCGGTGGCGGCCGCCCGGGCAGGTACGCGGGGACCTGCTGTGCTCTTGCTTGCACA
GTGTCCTGGAGCTGGACCTGGCTCTGGGTTTCCAGGAAGCAGTTTGACTAAAGGCAGCAAGCTGCT
TCCTCTGCTGCCTGAGATACCANATTCCCAATGGCNAANATTGANAAAAACGCTCCCACGATGGA
AAAAAAGCCANAACTGTTTAACATCATGGAAGTAGATGGAGTCCCTACGTTGATATTATCAAAAG
AATGGTGGGAAAAAGTATGTAATTTCCAAGCCAAGCCTGATGATCTTATTCTGGCAACTTACCCAA
AGTCAGGTACCT

| | |
|---|---|
| Sequence 823 | cMhvSB101e07a2 |

GGGCNAATTGGAGCTCCCCGCGGTGGCGGCCGANGTACACGNGGTNTAACCTGCTGNNNTCTTGN
TTGCACAGTGNCCNGGATCTGGACCTGGCTCTNGGTTGGGNGGANNCNNTCCGACTAANGGCACC
NTNCTGNTTNNTNTGNTGNCTNANNTNCCATATTCCNNNTGGNAAATATTGACAAAAACGCTCCCA
CGATGGAAAAAAAGCCAGAACTGTTTAACATCATGGAAGTAGATGGAGTCCCTACGTTGATATTA
TCAAAAGAATGGTGGGAAAAAGTCTGTAATTTCCAAGCCAAGCCTGATGATCTTATTCTGGCAACT
TACCCAAAGTCAGGTACCTGCCCG

| | |
|---|---|
| Sequence 824 | cMhvSB105f02a2 |

AGGTACGCGGGGACCTCACCTGCTGTGCTCTTGCTTGCACAGTGTCCTGGAGCTGGACCTGGCTCT
GGGTTTCCAGGAAGCAGTTTGACTAAAGGCAGCAAGCTGCTTCCTCTGCTGCCTGAAATACCAGAT
TCCCAATGGCGAAGATTGAGAAAAACGCTCCCACGATGGAAAAAAAGCCAGAACTGTTTAACATC
ATGGAAGTAGATGGAGTCCCTACGTTGATATTATCAAAAGAATGGTGGGAAAAAGTCTGTAATTT
CCAAGCCAAGCCTGATGATCTTATTCTGGCAACTTACCCAAAGTCAGGTACCTGCCCG

| | |
|---|---|
| Sequence 825 | cMhvSB105h07a2 |

AGGTACCTGACTTTGGGTAAGTTGCCAGAATAAGATCATCAGGCTTGGCTTGGAAATTACATACTT
TTTCCCACCATTCTTTTGATAATATCAACGTAGGGACTCCATCTACTTCCATGATGTTAAACAGTTC
TGGCTTTTTTTCCATCGTGGGAGCGTTTTTCTCAATCTTCGCCATTGGGAATCTGGTATCTCAGGCA
GCAGAGGAAGCAGCTTGCTGCCTTTAGTCAAACTGCTTCCTGGAAACCCAGAGCCAGGTCCAGCTC
CAGGACACTGTGCAAGCAAGAGCACAGCAGGTGACCCCGCGTACCTGCCCG

| | |
|---|---|
| Sequence 826 | cMhvSB027b01a2 |

CCCTTAGCGTGGTCGCGGCCGAGGTACCGCAGTATGGTTGGCCATGGGATTATCCTTCATTACATC
AAATGAGGTATGGTGGACAATCTTGTTTATAACATCACCTGACAAAGTTTTCTCCAAGAATTCCAA
CACCTTGTGGATCTCATGTTTTGGATTTTTTTTAATATCCCCGTAGAAGAGGTAGAGGATCCGGTGC
GTGTCTTTTGCAGCCCACCATCCTTTCACATGGTCAAACCAGGACCTGCCAACAACTTTTCCGGAC
ATGAATTTCTCATAAAATTCCTCTAAGTTCTGAGGATCAGGCATAAAGGAAGCCATCCTGTGAAAG
TGGTAGTAGGACACCAGGCAATCCTTGGGATTTCTGGCCACATAGACAATCTTGCAGTTTTCTTTC
CAGATAGATGGTGGAATCAGATGTGAAGGGAGATGTGTTTT

| | |
|---|---|
| Sequence 827 | cMhvSB027g10a2 |

GGTACCGCAGTATGGTTGGCCATGGGATTATCCTTCATTACATCAAATGAGGTATGGTGGACAATC
TTGTTTATAACATCACCTGACCAAGTTTTCTCCAAGAATTCCAACACCTTGTGGATCTCATGTTTTG
GATTTTTTTTAATATCCTCGTAGAAGAGGTAGAGGATCCGGTGCATGTCTTTTGCAGCCCACCATCC
TTTCACATGGTCAAACCAGGACCCGCCAACAACTTTTCCGGACATGAATTTCTCATAAAATTCCTC
TAAGTTCTGAGGATCAGGCATAAAGGAAGCCATCCTGTGAAAGTGGTAGTAGGACACCAGGCAAT
CCTTGGGATTTCTGGCCACATAGACAATCTTGCAGTTTTCTTTCCAGATAGATGGTGGAATCAGAT
GTGAAGGGAGATG

| | |
|---|---|
| Sequence 828 | cMhvSB031h02a2 |

CCCTTAGCGTGGTCGCGGCCGAGGTACCGCAGTATGGTTGGCCATGGGATTATCCTTCATTACATC
AAATGAGGTATGGTGGACAATCTTGTTTATAACATCACCTGACCAAGTTTTCTCCAAGAATTCCAA
CACCTTGTGGATCTCATGTTTTGGATTTTTTTTAATATCCTCGTAGAAGAGGTAGAGGATCCGGTGC
ATGTCTTTTGCAGCCCACCATCCTTTCACATGGTCAAACCAGGACCCGCCAACAACTTTTCCGGAC
ATGAATTTCTCATAAAATTCCTCTAAGTTCTGAGGATCAGGCATAAAGGAAGCCATCCTGTGAAAG
TGGTAGTAGGACACCAGGCAATCCTTGGGATTTCTGGCCACATAGACAATCTTGCAGTTTTCTT

| | |
|---|---|
| Sequence 829 | cMhvSB038f11a2 |

CGGCCGCCCGGGCAGGTACCGCAGTATGGTTGGCCATGGGATTATCCTTCATTACATCAAATGAGG
TATGGTGGACAATCTTGTTTATAACATCACCTGACCAAGTTTTCTCCAAGAATTCCAACACCTTGTG
GATCTCATGTTTTGGATTTTTTTTAATATCCTCGTAGAAGAGGTAGAGGATCCGGTGCATGTCTTTT
GCAGCCCACCATCCTTTCACATGGTCAAACCAGGACCCGCCAACAACTTTTCCGGACATGAATTTC
TCATAAAATTCCTCTAAGTTCTGAGGATCAGGCATAAAGGAAGCCATCC

| | |
|---|---|
| Sequence 830 | cMhvSB065c08a2 |

CGCCCGGGCAGGTACCGCAGTATGGTTGGCCATGGGATTATCCTTCATTACATCAAATGAGGTATG
GTGGACAATCTTGTTTATAACATCACCTGACCAAGTTTTCTCCAAGAATTCCAACACCTTGTGGATC
TCATGTTTTGGATTTTTTTTAATATCCTCGTAGAAGAGGTAGAGGATCCGGTGCATGTCTTTTGCAG
CCCACCATCCTTTCACATGGTCAAACCAGGACCCGCCAACAACTTTTCCGGACATGAATCTCTCAT
AAAATTCCTCTAAGTTCTGAGGATCAGGCATAAAGGAAGCCATCCTGTGAAAGTGGTAGTAGGAC
ACCAGGCAATCCTTGGGATTTCTGGCCACATAGACAATCTTGCAG

| | |
|---|---|
| Sequence 831 | cMhvSB071c02a2 |

NNATTGGAGCTCCACCGCGGTGGCCGAGCGGCCGCCCGGGCAGGTACGCGGGTAGACACGCTTTC
CTTGAACTGAAATTTTCCCCATAAAGAAAAACCAGATTTGGAGTTCGTTCTTGAAATGTCCTCACC
ACAACTGATAAAAACACATCTCCCTTCACATCTGATTCCACCATCTATCTGGAAAGAAAACTGCAA
GATCGTCTATGTGGCCAGAAATCCCAAGGATTGCCTGGTGTCCTACTACCACTTTCACAGGATGGC
TTCCTTTATGCCTGATCCTCAGAACTTAGAGGAATTTTATGAGAAATTCATGTCCGGAAAAGTTGTT
GGCGGGTCCTGGTTTGACCATGTGAAAGGATGGTGGGCTGCAAAAGACA

| | |
|---|---|
| Sequence 832 | cMhvSB073d08a2 |

TGATTGGAGCTCCCCGCGGTGGCCGAGCGGCCGCCCGGGCAGGTACAACATGGATGCATGAAATT
TTAGACATGATTCTAAATGATGGTGATGTGGAGAAATGCAAAAGAGCCCAGACTCTAGATAGACA
CGCTTTCCTTGAACTGAAATTTCCCCATAAAGAAAAACCAGATTTGGAGTTCGTTCTTGAAATGTC
CTCACCACAACTGATAAAAACACATCTCCCTTCACATCTGATTCCACCATCTATCTGGAAAGAA

| | |
|---|---|
| Sequence 833 | cMhvSB082e04a2 |

ACCGCAGTATGGTTGGCCATGGGATTATCCTTCATTACATCAAATGAGGTATGGTGGACAATCTTG
TTTATAACATCACCTGACCAAGTTTTCTCCAAGAATTCCAACACCTTGTGGATCTCATGTTTTGGAT
TTTTTTAATATCCTCGTAGAAGAGGTAGAGGATCCGGTGCATGTCTTTTGCAGCCCACCATCCTTTC
ACATGGTCAAACCAGGACCCGCCAACAACTTTTCCGGACATGAATTTCTCATAAAATTCCTCTAAG
TTCTGAGGATCAGGCATAAAGGAAGCCATCCTGTGAAAGTGGTAGTAGGACACCAGGCAATCCTT
GGGATTTCTGGCCACATAGACAATCTTGCAGTTTTCTTTCCAGATAGATGGTGGAATCAGATGTGA
AGGGAGATGTGTTTTTATCAGTTGTGGTGAGGACATTTCAAGAACGAACTCCAAATCTGGTTTTTC
TTTATGGGGAAATTTCAGTTCAAGGAAAACGTGTCTATCTANAAGTCTGGGCTCTTTTGCATTTCTC
CACATCACCATCATTTAGAATCATGTCTAAAATTTCATGCATCCATGTTGTACCTCGGCCCGCTCTA
NAAACTAGNGGGATC

| | |
|---|---|
| Sequence 834 | cMhvSB082g02a2 |

AGGTACAACATGGATGCATGAAATTNTANACNTGATTCTAAATGATGGTGATGTGGANAAATGCN
AAAGAGCCCAGACTCTAGATAGACACGCTTTCCTTGAACTGAANTTTCCCCATAACAGAAAAACC
AGATTTGGAGTTCGTTCTTGAAATGTCCTCACCACAACTGATAAAAACACATCTCCCTTCACATCT
GATTCCACCATCTATCTGGAAAGAAAACTGCAAGATTGTCTATGTGGCCAGAAATCCCAAGGATTG
CCTGGTGTCCTACTACCACTTTCACAGGATGGCTTCCTTTATGCCTGATCCTCAGAACTTAGAGGAA
TTTTATGAGAAATTCATGTCCGGAAAAGTTGTTGGCGGGTCCTGGTTTGACCATGTGAAGGGATGG
TGGGGCTGCAAAAAGACATGCACCGGATCCTCTTACCTCTTCTACGAGGGATATTAAAAAAAATCC
CAAAAACCATGAGATCCCCAAAGGTGGTTGGAATTCTTGG

| | |
|---|---|
| Sequence 835 | cMhvSB092b01a2 |

CCGGGCAGGTACCGCAGTATGGTTGGCCATGGGATTATCCTTCATTACATCAAATGAGGTATGGTG
GACAATCTTGTTTATAACATCACCTGACCAAGTTTTCTCCAAGAATTCCAACACCTTGTGGATCTCA
TGTTTTGGATTTTTTTTAATATCCTCGTANAAGAGGTAGAGGATCCGGTGCATGTCTTTTGCAGCCC
ACCATCCTTTCACATGGTCAAACCAGGACCCGCCAACAACTTTTCCGGACATGAATTTCTCATAAA
ATTCCTCTAAGTTCTGAGGATCTGGCATAAAGGAAGCCATCCTGTGAAAGTGGTAGTAGGACACC
AGGCAATCCTTGGGATTTCTGGCCACATAGACAATCTTGCAGTTTTCTTTCCAGATAGATGGTGGA
ATCAGATGTGAAGGGAGATGTGTTTTTATCAGTTGTGGTGAGGACATTTCAAGAACGAACTCCAAA
TCTGGTTTTTCTTTATGGGGAAATTTCAGTTCAAGGAAAGCGTGTCTATCTAGAGTCTGGGCTCTTT
TGCATTTCTCCCATCACCATCATTTAAAATCATGTCTAAAATTTCATGCATCCATGTTGTACCTCGC
CGTCTAGAACTAGTGGATCCCCGGGCTGCAGGAATTCNAT

| | |
|---|---|
| Sequence 836 | cMhvSB092d06a2 |

AGGTACAACATGGATGCATGAAATTTTAGACATGATTCTAAATGATGGTGATGTGGAGAAATGCA
AAAGAGCCCAGACTCTAGATAGACACGCTTTCCTTGAACTGAAATTTCCCCATAAAGAAAAACCA
GATTTGGAGTTCGTTCTTGAAATGTCCTCACCACAACTGATAAAAACACATCTCCCTTCACATCTG
ATTCCACCATCTATCTGGAAAGAAAACTGCAAGATTGTCTATGTGGCCAGAAATCCCAAGGATTGC
CTGGTGTCCTACTACCACTTTCACAGGATGGCTTCCTTTATGCCTGATCCTCAGAACTTAGAGGAAT
TTTATGAGAAATTCATGTCCGGAAAAGTTGTTGGCGGGTCCTGGTTTGACCATGTGAAAGGATGGT
GGGCTGCAAAAGACATGCACCGGATCCTCTACCTCTTCTACGAGGATATTAAAAAAAATCCAAAA
CATGAGATCCACAAGGTGTTGGAATTCTTGGANAAAACTTGGTCAGGTGATGTTATAAACAAAGA
TTGTCCACCATACCTCA

| | |
|---|---|
| Sequence 837 | cMhvSB093f05a2 |

GATTGGAGCTCCCCGCGGTGGCGGCCGCCCGGGCAGGTACAACATGGATGCATGAAATTTTAGAC
ATGATTCTAAATGATGGTGATGTGGAGAAATGCAAAAGAGCCCAGACTCTAGATAGACACGCTTT
CCTTGAACTGAAATTTCCCCATAAAGAAAAACCAGATTTGGAGTTCGTTCTTGAAATGTCCTCACC
ACAACTGATAAAAACACATCTCCCTTCACATCTGATTCCACCATCTATCTGGAAAGAAAACTGCAA
GATTGTCTATGTGGCCAGAAATCCCAAGGATTGCCTGGTGTCCTACTACCACTTTCACAGGATGGC
TTCCTTTATGCCTGATCCTCAGAACTTAGAGGAATTTTATGAGAAATTCATGTCCGGAAAAGTTGTT
GGCGGGTCCTGGTTTGACCAT

| | |
|---|---|
| Sequence 838 | cMhvSB093f12a2 |

CGGGCAGGTACAACATGGATGCATGAAATTTTAGACATGATTCTAAATGATGGTGATGTGGAGAA
ATGCAAAAGAGCCCAGACTCTAGATAGACACGCTTTCCTTGAACTGAAATTTCCCCATAAAGAAA
AACCAGATTTGGAGTTCGTTCTTGAAATGTCCTCACCACAACTGATAAAAACACATCTCCCTTCAC
ATCTGATTCCACCATCTATCTGGAAAGAAAACTGCAAGATTGTCTATGTGGCCAGAAATCCCAAGG
ATTGCCTGGTGTCCTACTACCACTTTCACAGGATGGCTTNCTTTATGCCTGATCCTCAGAACTTAGA
GGAATTTTATGAGAAATTCATGTCCGGAAAAGTTGTTGGCGGGTCCTGGTTTGACCATGTGAAAGG
ATGGTGGGCTGCAAAAGACATGCACCGG

| | |
|---|---|
| Sequence 839 | cMhvSB026g10a2 |

CCCTTTCNAGCGGCCGCCCGGGCNGGTACTGANCTCCACAAACGTGGCCATGGTTGGTGCGGAAA
TGATTCTGANTGAGCAGGTAAAAGNCTCACGTNCTGCTGTGTCCANAGTTGGTTCCTTCCANAGGG
TTCGTGGTCTNGCTGGCTTCAAGAATGAAGCCGTGGACCTTCACAGTGTGTGTNACANCTGTTAAA
GATGTNGTGTCTGGANTNACGTTCCTTCACATGTGTCTGGA

| | |
|---|---|
| Sequence 840 | cMhvSB027c01a2 |

ACGCGGGGAGGAGAGATCAAACAGAACTGCTGCTGGGTGGTTGTCAGGAGCTGCTACACGGAGA
ACCCTGGACTATTCGATCAAGCAGCAAGGCTATATGTTCACTTATGCAGAAATGGACCATTGCAGA
TGCTAATCTTTGTTGTGCAAGCGAAGGCTCACTTGGAAGGAAATACTCAGCCCCTCTCTGGGCAGC
ATTTGAGTTCCTTATGGATACCGAGTCGCGAAACAAGTTATTTTTTTTAATGTATCCTTCTTTATGA
GGAGAATGCTACCCAAAAATGTATTAAAGGAATATTAAGTCGTCCAGAGACTGTCTTGCTACCAA
GAACTGTGCAATGGAATTCTTTTT

| | |
|---|---|
| Sequence 841 | cMhvSB028a04a2 |

ACCTCAGTTGGAAATGCAGAAATCACCCATCTTCTACATCGATCTTGCTGGGAGCTGCAGACCAGA
GCTGTTCCTATTTGGCTATCTTGGAAGCAACCTCAGGTATTTCTTTATTAGCAGTGTGAGAACAGAC
TAATACAGATTACTAAATCCAGAATCCAGAGAACACAAGATTATAAGTTCCTTGCGCTTGAGCATG
TTCAGTGAGAGCGCTGCAGGGAGAAGGATGATGCATTCTGAGAGCCAACAGGGCTGGACTGGAAA
CTGGAGGAAGAGAAAGAGCTAAGGAAGGAGAGGAGCAAATTGG

| | |
|---|---|
| Sequence 842 | cMhvSB029g05a2 |

CCCTTAGCGTGGTCGCGGCCGAGGTACCTGACTTNGGGTANGTNGCCATANTANGANCATNANGC
NTGGNTNGGAAANTACATACTTTTTCCCACCATTCTTTTGATAANATCAACNTATGGACTNCNTCT
ACTTNCATGATNTNAAACANTANTGGNTTTTTTTNCNTNGNGGGAGCGTNTTTCTCANTCTTNACN
ATTGGGAATCAGATGGGCTTTTGGCTTATCTCTCCCTGTGTGAGCCATTAAAGGGGATAATAAGGA
TCATTGCTTATATTCTCTGTGAATTTATAATTAATGAAAAAGGATT

| | |
|---|---|
| Sequence 843 | cMhvSB031g06a2 |

CCCTTAGCGTGGTCGCGGCCGAGGTACGCGGGCTTTNAAGAAGTCCTTGTTGGAATTTTCCTNAGC
TAGATTTCAAGCCATGTCAGGACACCACTCTCATTATATTACCATAATNGNTTTTTCTTTNTTTTTTT
TTNAAATTTNANTTTTTTAAAATTCCNGGATNCATGNNCNGNANNNNCCNTATTTTTTTTAANGTC
AAATCCNNCNTTANTNTCCNNGTNGATNACAAATATAACCCNGAGGNAATTTTTTTTTTTTTTTTTT

| | |
|---|---|
| Sequence 844 | cMhvSB044b07a2 |

AGGTACTGTGGGTTCTGAGTCAAGGATCCCAGTGCTGCCAGGAACCAGCAGTCAGCTGCGCCTCCT
TGTTGGATGTCAAATCTGCTTATATCATCCAGGATGAAGTGAGGAGGACCCCCTGGTAGATCCTGT
GGCCGCTTCCATATCACATTGGAGAGGCGTTTTTCCTGGAGCAGCTTCTGGCCTATGGAAGAATCT
GCTGCAGGGAATGTCTCATCCTTAAACGTCCGGCCCATGCTCAGGCAGTGATCCCGCAAGGTGGTA
AAGTCCTGGTCTTTGAACTTGATGATGGAGGTCTCCACTGAAGGCTCCTGGTAATACGCCATGACT
CTCCTTAGAA

| | |
|---|---|
| Sequence 845 | cMhvSB044f11a2 |

TNAGGGCGAATTGGAGCTCCCCGCGGTGGCGGCCGCCCGGGCAGGTACTGATCATAGTTGATCAC
AATTGGAGGGGGAAGGGCTGTGGCTTCTCAAATCAAAGGAGGCTGGTGGGTTAAAATCATCAACA
GCATTTCATGGTCTTAAGTTCACTTCTCATCAAAGGATCCCTGTGGTGTTGTGTTTGATCTTGAACT
TGCTTTGCTGTTTTAGTCTTTTCCATCAAGTGAAAAAACTACTTTGATGACATCCTTTCATCCTTTTC
ATTCTTCCAGCAGTATTTGGTATCTTTTTACACTAGGCTTGCAGGCCAGGGAATCTGCATGCCAGTC
TCCACTTGCTGAACAGCAGTTTTCTTTTGATCATATTCGGTTTTAGGACACTTGAGGC

| | |
|---|---|
| Sequence 846 | cMhvSB049c12a2 |

AGGTACTGTGGGTTCTGAGTCAAGGATCCCAGTGCTGCCAGGAACCAGCAGTCAGCTGCGCCTCCT
TGTTGGATGTCAAATCTGCTTATATCATCCAGGATGAAGTGAGGAGGACCCCCTGGTAGATCCTGT
GGCCGCTTCCATATCACATTGGAGAGGCGTTTTTCCTGGAGCAGCTTCTGGCCTATGGAAGAATCT
GCTGCAGGGAATGTCTCATCCTTAAACGTCCGGCCCATGCTCAGGCAGTGATCCCGCAAGGTGGTA
AAGTCCTGGTCTTTGAACTTGATGATGGAGGTCTCCACTGAAGGCTCCTGGTAATACGCCATGACT
CTCCTTAGAAGACTTC

| | |
|---|---|
| Sequence 847 | cMhvSB049e07a2 |

CCGGGCAGGTACGCGGGGAAAGTGTGTAGCACCTCCACCTTCTCTCTCTCTCTCCCTCTCCCTCTCC
TGCCAGCCAAGTGAAGACATGCTTACTTCCCCTTCACCTTCCTTCATGATGTTACCATTGGAATGAC
ATACTGCATCCTATAGTTATACCATCCACTCTGAAATCAATGTGAATTTAACTTCAGTTCCATACAG
AAACTTCTTTTCCACAGGTAAGAAACGGTTGAACTGGATGCAATTTTTATCACAGCTTGTGTAAGA
CTGCCTCTGTCCCTCCTCTCACATGCCATTGGTTAACCAGCAGACAGTGTGCTCGGGGGCGTTGCC
AGCTCATTGCTCTTATA

| | |
|---|---|
| Sequence 848 | cMhvSB049h11a2 |

AGGTACTGTGGGTTCTGAGTCAAGGATCCCAGTGCTGCCAGGAACCAGCAGTCAGCTGCGCCTCCT
TGTTGGATGTCAAATCTGCTTATATCATCCAGGATGAAGTGAGGAGGACCCCCTGGTAGATCCTGT
GGCCGCTTCCATATCACATTGGAGAGGCGTTTTTCCTGGAGCAGCTTCTGGCCTATGGAAGAATCT
GCTGCAGGGAATGTCTCATCCTTAAACGTCCGGCCCATGCTCAGGCAGTGATCCCGCAAGGTGGTA
AAGTCCTGGTCTTTGAACTTGATGATGGAGGTCTCCACTGAAGGCTCCTGGTAATACGCCATGACT
CTCCTTAGAAGACTTCCGAGGT

| | |
|---|---|
| Sequence 849 | cMhvSB063a08a2 |

ATTGGAGCTCCCCGCGGTGGCGGCCCGAGGTACTGTGGGTTCTGAGTCAAGGATCCCAGTGCTGCC
AGGAACCAGCAGTCAGCTGCGCCTCCTTGTTGGATGTCAAATCTGCTTATATCATCCAGGATGAAG
TGAGGAGGACCCCCTGGTAGATCCTGTGGCCGCTTCCATATCACATTGGAGAGGCGTTTTTCCTGG
AGCAGCTTCTGGCCTATGGAAGAATCTGCTGCAGGGAATGTCTCATCCTTAAACGTCCGGCCCATG
CTCAGGCAGTGATCCCGCAAGGTGGTAAAGTCCTGGTCTTTGAACTTGATGATGGAGGTCTCCACT
GAAGGCTCCTGGTAATACCCCATGACTCTCCTTAGAAGACTTCCGAGGTCCTTTCCTGTTTCCTANG
CAGGTGTGTCTGATGGAGGAGGGGAGACCGGCAGGT

| | |
|---|---|
| Sequence 850 | cMhvSB063f02a2 |

NTCCTTTTTTTTTTTAATTTTTAAATCAGCTTTCCTAGCTNGAAGNGTTNCTAGTNTTGAATGGTGG
GATGTAGTCAAGGAGGTNTTTGTTCAAGGTTGGANATGANCAGCTTTTATAATAATTCCAGGTTTG
GGATATATCAGNGAAATTTCATTTTTCATTTTCTACTAACAGNGCCANATNGGCCTCACTTTTTGGA
CTGGATCAGGCAGCTGCTGGCCATGGAAATGAATTTTTCCAGTACACAGCCCCA

| | |
|---|---|
| Sequence 851 | cMhvSB071c10a2 |

ACGCGGGGATGAGATCTGGTTGTTTGAAAGTGTGTAGCACCTCCACCTTCTCTCTCTCCCTCCCTCT
CCCTCTCCTGCCAGCCAAGTGAAGACATGCTTACTTCCCCTTCACCTTCCTTCATGATGTTACCATT
GGAATGACATACTGCATCCTATAGTTATACCATCCACTCTGAAATCAATGTGACTTTAACTTCAGTT
CCATACAGAAACTTCTTTTCCACGGGTAAGAAACGGTTGAACTGGATGCAATTTTTATCACAGCTT
GTGTAAGACTGCCTCTGTCCCTCCTCTCACATGCCATTGGTTAACCAGCAGACAGTGTGCTCAGGG

| | |
|---|---|
| Sequence 852 | cMhvSB071g06a2 |

CTNATTGGAGCTCCACCGCGGTGGCCTACCGGAACTGAATCTGCCTTCCAAGTTACACGGATAAGA
ATTATGGTTCGACGTGGTGGCATCGGTGCCCAGTGTGGGTTGGTGTTTGCCTATAACTCATCTTCAG
ATAAATTTTGTGCAGGAAGAACACTTCAAAAGGTTTGAAAAATATGACAAATGGAAGCTTCAGGA
GCTCAGGCAATTTGTAAAAAGCAGGTAAGAAGGTAAAAAATCTTTGTAGAACAAAGATCTACAGA
ACAAAAATCTTTGTAGTTAATAAGAATGTATTCATGCTCATTGGTGAACTGTGCTTGCTTGTCTTTA
TAGAAAAGGCGCCACTAATCCATCTCAGTGGCCATAAGCCTTCATT

| | |
|---|---|
| Sequence 853 | cMhvSB073b07a2 |

ATGTACACCNGGTNANNANCNTGGCCTGNGGCNGTANGNNCTCATGNTCATCTNTNNNTGGAAAN
NCCTAGGGNGGCNCAGGGNCAACANTTTNNNACANNANCTGANGGTNAAACGGCCTNTNGCNGA
CTTAANNCTCATGCCTGTNAATTGGAAATACAAAGACCTCCAAAAAAGGACCAGTTCCTCGGATG
TGCCCCCTCACAGAGAGATGAAGGGGCAGCAGAAAACAGCTGAAACGGAAGAGGGGACAGTGCA
GATTCAGGAAGGTGCAGTGGCTACTGGGGAAGACCCAACCAGTGTGGCTATTGCCAGCATCCANT
CAGCTGCCACCTTCCTGACCCCAACGTCAAGTGATGTACCTGCCCGGGCGGCCGCTCG

| | |
|---|---|
| Sequence 854 | cMhvSB073b11a2 |

AGCTCCCCGCGGTGGCGGCCGAGGTACTGTCGTTGGGTTGCACCCAAGGCACTTGGGCCCACCTGC
CTTCCCACACACTCACTATCCAGAAAAGAGGAAAAGCCTAAAGATGACACACCTTCCTCCCTACTC
AGGCCTCCTCGGCGATGGCTTTGATTGTCTTGTGTTTTTTATAGGGGCCAAAGAGCAGTTGATTTTT
TTTCAAAGTCTAGTATTTCTCTGAAGATTCTACATCTCTACACAAGATATTCATTCTTTTGGTCACC
TAGGGATCTTCTAAGTGTGATATTACTTTCAGAGAATTCAGACAAGTGAGAAACAATAATGTAGG
AGTCAGCAAAG

| | |
|---|---|
| Sequence 855 | cMhvSB075c01a2 |

CTGATTGGAGCTCCCCGCGGTGGCGGCCCGAGGTACGCGGGGAGACTCTGCCTTTTCAACATGGAT
GGCTCCTCCCGCTGCCGCTGCCGCTCCAGGAGACAGCATTACAGAGCATCAGTTAGGTGCAGAGA
CTGGGCAGTGCGCCCGTGTGCAAAGACAGGAGACACGAATCTTCCCTGAAGGAGTGACAGTCTAG
GGAGGAAGGCAGACTGCAGGGGACCTACTTCTCTCGGGAATCTCAATACTTGGAACAAGAACCTC
CTAGACGGACCCTTTGGCATAATGAATTGGACCAACTGTAGGTTCCAGGACTAGAGAGCCAGCAA
TGCCTCCATGAACAATCTCACCCAATTACTCTGCTCAGGAAACGAGGTAACTGATGGACAGCCGA
GGCAGCCCCTT

| | |
|---|---|
| Sequence 856 | cMhvSB075e02a2 |

CGAGGTACTGTGGGTTCTGAGTCAAGGATCCCAGTGCTGCCAGGAACCAGCAGTCAGCTGCGCCT
CCTTGTTGGATGTCAAATCTGCTTATATCATCCAGGATGAAGTGAGGAGGACCCCCTGGTAGATCC
TGTGGCCGCTTCCATATCACATTGGAGAGGCGTTTTTCCTGGAGCAGCTTCTGGCCTATGGAAGAA
TCTGCTGCAGGGAATGTCTCATCCTTAAACGTCCGGCCCATGCTCAGGCAGTGATCCCGCAAGGTG
GTAAAGTCCTGGTCTTTGAACTTGATGATGGAGGTCTCCACTGAAGGCTCCTGGTAATACGCCATG
ACTCTCCTTAAAAGACTTCCGAGGTCCT

| | |
|---|---|
| Sequence 857 | cMhvSB079a10a2 |

CCGGGCAGGTACAGGACACAATCCCTGCTTCATTCTTGGCTGACACAGTATACCACCCAGCATCTT
CTTTTGTGGCTCCCTGAATGAGCAGGCAGATGTAGCCGTGGTTGTCCTGGTGCATGCTGGAGAAAA
GGATAAAGTCATTAGGGTTCTAAATTTTTTAAAAGTGGCTTTGGACATGAAGCATCATTTTTAATT
AGATCATTAGAAACAGAATTGTGCAAGTAGCTGATAATAGGGTCATACTTATTCTGTAGAGATTAC
TAGCTCCATTAAAGTTAATGGGAGAAAGAACAGACGTCAAGAGTTGAATACATCTGTGTGCTTAA
TTCCTAGTTGAGGATCTGCCTTTACAAAAACCACTGAATAGTCTTTTATCACTAAAGCAAATGAAT
TCATCTTTTCTTTTAGATAGAATGATAAACA

| | |
|---|---|
| Sequence 858 | cMhvSB079c06a2 |

TTNTNNCANTCTNATCAGATACNTGGCCGACCTCCNAGGGGGGGCCCGGGNACCGNGACTNTTGT
CNCATTNAGTGAGGNNCAATCNGGAGGCTTGGCCGTANNTNTGGACCATATCTGGTTCTCNTGCTC
CATGAGAAAAGTTTTAGAGACAGTCTTTGATGAAGTCATCATGGTAGATGTCTTGGACAGTGGCGA
TTCTGCTCATCTAACCTTAATGAAGAGGCCAGAGTTGGGTGTCACGCTGACAAAGCTCCACTGCTG
GTCGCTTACACAGTATTCAAAATGTGTATTCATGGATGCAGATACTCTGGTCCTA

| | |
|---|---|
| Sequence 859 | cMhvSB080a05a2 |

ACGCGGGGAGACTCTGCCTTTTCAACATGGATGGCTCCTCCCGCTGCCGCTGCCGCTCCAGGAGAC
AGCATTACAGAGCATCAGTTAGGTGCAGAGACTGGGCAGTGCGCCCGTGTGCAAAGACAGGAGAC
ACGAATCTTCCCTGAAGGAGTGACAGTCTAGGGAGGAAGGCAGACTGCAGGGGACCTACTTCTCT
CGGGAATCTCAATACTTGGAACAAGAACCTCCTAGACGGACCCTTTGGCATAATGAATTGGACCA
ACTGTAGGTTCCAGGACTAGAGAGCCAGCAATGCCTCCATGAACAATCTCACCCAATTACTCTGCT
CAGGAAACGA

| | |
|---|---|
| Sequence 860 | cMhvSB080g07a2 |

CNAATTGGAGCTCCCCGCGGTGGCGGCCGAGGTACTGTGGGTTCTGAGTCAAGGATCCCAGTGCT
GCCAGGAACCAGCAGTCAGCTGCGCCTCCTTGTTGGATGTCAAATCTGCTTATATCATCCAGGATG
AAGTGAGGAGGACCCCCTGGTAGATCCTGTGGCCGCTTCCATATCACATTGGAGAGGCGTTTTTCC
TGGAGCAGCTTCTGGCCTATGGAAGAATCTGCTGCAGGGAATGTCTCATCCTTAAACGTCCGGCCC
ATGCTCAGGCAGTGATCCCGCAAGGTGGTAAAGTCCTGGTCTTTGAACTTGATGATGGAGGTCTCC
ACTGAAGGCTCCTGGTAATACGCCATGACTCTCCTTAGAAGACTTCCGAGGTCCT

| | |
|---|---|
| Sequence 861 | cMhvSB082a03a2 |

NCNNGCNTNGTCCTATATCNAATATACCCATTGCGGGCCNNGCCNCTNGGAGGNCTNTTCTCANNT
NANNNNATCATNCGNTGANGGTGGCNTTAGATCCNAANTATCNCCCCNTTGACTGTGCNTATNNN
TNTNAGANNCTGCANCAAGCGGGATAANCCNTTNATNATAATATCCNNNATAAGGNTGNGATNCT
NNAGNNNCTGTGCNTCTGNTGGNNAGTAGTGANCTCTTTCTTTACCAGACCCCTNGTGGACGAAN
GCTTTTATACAAGACCCTCCTGGACCNTGCAGCTATACNNTATGNACCTGNATCNNNTNCCCTGNC
CNNNNNGNTNCCTGACNGGGGATGACTTTTTCCCCAAAGATGATAAAGGTAATATGATCAGTGGA
AAAGGAACGTTCTTGGATGCCTGGGAGGCCATGGAGGAGCTGGTGGACGAGGGGCTGGTGAAAG
CCCTTGGGGTCTCAAATTTCAACCACTTCCAGATCGAGAGGCTCTTGAACAAACCTGGACTGGAAA
TATAAACCAGTGACTAACCAGGTTTGAGTGTCACCCATACCTCACGCCAGGANAAACTGATCCAGT
CCTCGGCCCGTCTTAAAACTAGTGGATCCCCCCGGCTTGCAGGAAATTCGATTTCAAAGCTTATCG
ATNCCCGNCNACCTCNANGGGG

| | |
|---|---|
| Sequence 862 | cMhvSB082e03a2 |

GCCATGCTCTCCTCCTCTGCCAGTCTCCTCCACCACTCTCTAACCTGAGAGCCTGTGGAACCTGCCC
GTCTCCCCTCCTCCATCAGACACACCTGCCTAGGAAACAGGAAAGGACCTCGGAAGTCTTCTAAGG
AGAGTCATGGCGTATTACCAGGAGCCTTCAGTGGAGACCTCCATCATCAAGTTCAAAGACCAGGA
CTTTACCACCTTGCGGGATCACTGCCTGAGCATGGGCCGGACGTTTAAGGATGAGACATTCCCTGC
AGCAGATTCTTCCATAGGCCAGAAGCTGCTCCAGGAAAAACGCCTCTCCAATGTGATATGGAAGC
GGCCACAGGATCTACCAGGGGGTCCTCCTCACTTCATCCTGGATGATATAAGCAGATTTGACATCC
AACAAGGAGGCGCAGCTGACTGCTGGTTCCTGGCAGCACTGGGATCCTTGACTCANAACCCACAG
TACCT

| | |
|---|---|
| Sequence 863 | cMhvSB083d09a2 |

CCGGGCAGGTACCTGAAAAACAGCTGGTAGGATGGAGGAACTGAGCTTTTAAATAGGCAAATGTG
GCTAGGAGCTACCATACTGGACAGCACAGTGTATTAGTTTGGTGCAAAAGTAATTGTGGTTTTGGC
CATTTTTAAGTGGATTGGTAAGCCTGGCTATTTAAAGTGTGGTCCACAGAGCAGGAGAATCACTGC
ACCTGAGAGCTGGTGGAAATGTAGATCTCTGACGTTAGCATAGGCTTCCTAAATCAGAAACTGCAT
TCTAACAAGATCTCCTGGTGCTTCTCATGCACAGTAAAGTTTAGAAAGTTAGGAGATGCATACAAG
TGGTTCTCATCCTGACAGCACTTCAGACACAACTGAGAAACATTAAAAGAAGCTGAGCCTAGGTC
ACACCCTCCACCCAGAGATTCTTAGGTTAATGGTTTAAAGGCTTGGCCTGAACATGAAGAGTTTTA
AAAGCACTCTGGGGGATTCTAATAAAAATTCGAGAACCATCCCAGCATAAGTCAGTCCT

| | |
|---|---|
| Sequence 864 | cMhvSB090g05a2 |

NAATTGGAGCTCCCCGCGGTGGCGGCCGAGGTACTGTGGGTTCTGAGTCAAGGATCCCAGTGCTG
CCAGGAACCAGCAGTCAGCTGCGCCTCCTTGTTGGATGTCAAATCTGCTTATATCATCCAGGATGA
AGTGAGGAGGACCCCCTGGTAGATCCTGTGGCCGCTTCCATATCACATTGGAGAGGCGTTTTTCCT
GGAGCAGCTTCTGGCCTATGGAAGAATCTGCTGCAGGGAATGTCTCATCCTTAAACGTCCGGCCCA
TGCTCAGGCAGTGATCCCGCAAGGTGGTAAAGTCCTGGTCTTTGAACTTGATGATGGAGGTCTCCA
CTGAAGGCTCCTGGTAATACGCCATGACCCTCCTTAGAAGACTTCCGAGGTCCTTTCCTGTTTC

| | |
|---|---|
| Sequence 865 | cMhvSB090h07a2 |

NATTGGAGCTCCCCGCGGTGGCGGCCGCCCGGGCAGGTACCTGACTGTGGCTCAGATCTGCGTCGC
AGCAGCGAGAGAAGAAATCACTCCATATCCGATGAGAGGAAGAGTGGCACAGAGATGGTGTCTA
CAATTAGAGACATTTCTGACTCCACCTTAGCCTAAGCAAACTTTATATACTGAGTAACATTTGAAG
GTTGTCTTTTAATGGTGGGGGGTGTTTTTTTCCTTTTTAAACTACAGTGCTTGCACAAGAGAGGGAG
GGACTCANAAAAGGTTAGGGCAGGTGAGGGAGACAGTAGATGGCCTGGGATGACTTGAGTCCATC
ATACTATTGCTTTGGCGGGTGTCCTCCCCCATGTTTGATTCAAATTCCATGAGTGACCTACCTTTCC
CCAGGAATGGGACTGANAGGGTAAGTCTCCACAACTCAGTCTGCACAGGGCTCCCCGTTCAGGCT
GCCTT

| | |
|---|---|
| Sequence 866 | cMhvSB091d11a2 |

AGCTCCACCGCGGTGGCGGCCGGCCATGCTCTCCTCCTCTGCCAGTCTCCTCCACCACTCTCTAACC
TGAGAGCCTGTGGAACCTGCCCGTCTCCCCTCCTCCATCAGACACACCTGCCTAGGAAACAGGAAA
GGACCTCGGAAGTCTTCTAAGGAGAGTCATGGCGTATTACCAGGAGCCTTCAGTGGAGACCTCCAT
CATCAAGTTCAAAGACCAGGACTTTACCACCTTGCGGGATCACTGCCTGAGCATGGGCCGGACGTT
TAAGGATGAGACATTCCCTGCAGCAGATTCTTCCATAGGCCAGAAGCTGCTCCAGGAAAAACGCC
TCTCCAATGTGATATGGAAGCGGCCACAGGATCTACCAGGGGGTCCTCCTCACTTCATCCTGGATG
ATATAAGCAGATTTGACATCCAACAAGGAGGCGCAGCTGACTGCTGGTTCCTGGCAGCACTGGGA
TCCTTGAC

| | |
|---|---|
| Sequence 867 | cMhvSB091f05a2 |

GGAGCTCCCCGCGGTGGCGGCCGGCCATGCTCTCCTCCTCTGCCAGTCTCCTCCACCACTCTCTAAC
CTGAGAGCCTGTGGAACCTGCCCGTCTCCCCTCCTCCATCAGACACACCTGCCTAGGAAACAGGAA
AGGACCTCGGAAGTCTTCTAAGGAGAGTCATGGCGTATTACCAGGAGCCTTCAGTGGAGACCTCC
ATCATCAAGTTCAAAGACCAGGACTTTACCACCTTGCGGGATCACTGCCTGAGCATGGGCCGGAC
GTTTAAGGATGAGACATTCCCTGCAGCAGATTCTTCCATAGGCCAGAAGCTGCTCCAGGAAAAAC
GCCTCTCCAATGTGATATGGAAGCNGCCACAGGATCTACCAGGGGGTCCTCCTCACTTCATCCTGG
ATGATATAAGCAGATTTGACATCCAACAAGGAGGCGCAGCTGACTGCTGGTTCCTGGCAGCACTG

| | |
|---|---|
| Sequence 868 | cMhvSB092b02a2 |

NNATTGGAGCTCCCCGCGGTGGCGGAGATGTAGTCTTCACAGTGAGTTGTTATTTGTAGCTGTGTT
TTTGTTTTTGTATAGCTTATAGCAATGCAGTGTGCTTTTTATTAACATCATTTTCTTTTCTTTTTGCA
GTGATTATTTATTCAAGTTACTTCTGATTGGCGACTCAGGGGTTGGAAAGTCTTGCCTTCTTCTTAG
GTTTGCAGTAAGTTGAAATTGAAATGTCTTTACAATTAATGGTACAATTAATGCTATGTATGTTTTC
TAGGTAGATAAAATTAAACAGTTTTATTCAGAATAAGTTAATTCTTCCAGAATTTATATATTTAAA
GACTCCAAATATACATCCCCAGTGGTATCTTGGACTGTTAAATAGAAAAATATTGTTGCTCTTAAA
AGAAATTCAGTGAAGTCTGGTTATAAAGTCAGAATGTCTAATACTTTTGGTCAGAGTCAAACAGCA
GTTCCAATATAGGCAGCAAGTTAAAGGGGTAGTTGGTGGCCTGTGTTGAAAGCGACTTGATGAAA
ATAAATCTTTAAATTAAACTTTAGTAGAGCANANNNAAAAAAAAAA

| | |
|---|---|
| Sequence 869 | cMhvSB092f01a2 |

AGGTACAACTGCATACACGGAACTTTTGCCGTAACCACAACAAACGCCCATCCAGATGGCTCCGG
CTTAAGTTTCTATGCTTCACTAACCCCAAGGCCCACTAGTGCAGCCAGCAGTTGGGTTTTCCTCTTT
GGCAAGTCAGTCAGGCCATACAGAATCTGCTACAAGTTCCCTTCCTACCAGTTGAACTGTTTGCTG
AGCATGCAGGAATAGCCTCTGAATAGTATGGCCTGCTGTAAAGGGCAAGCTGGAAGTACCTGCCC
GGGCGGAATGATCAGGAGGAGACAGCCGGCGTTGTGTCCACCCCCCTCATTAGGAACGGTGACTG
GACCTTCCAGATCCTGGTGATGCTGGAAATGACTCCCCAGCGTGGAGATGTCTACACCTGCCACGT
GGAGCACCCCAGCCTCCAGAGCCCCATCACCGTGGAGTGGCGGGCTCAGTCTGAATCTGCCCAGA
NCAAGATGCTGAGTGGCGTTGGAGGCTTCGTGCTGGGGCTGATCTTCCTTGGGCTTGGCCTTATCA
TCCGTCAAANGAGTCGGAAAGGGCTTCTGCACTGACTCCTGAAACTGTTTAACTTAAGACTGGTTA
TCACTCTTTNTGTGATGCCTGTTTGTCC

| | |
|---|---|
| Sequence 870 | cMhvSB093f02a2 |

TGGAGCTCCCCGCGGTGGCGGCCGATGTACACCTNGNGCATNCAACCGNNTNCATGNNTTNCNNC
NCNNGCTAANCTATNCCCTTACCCTCTNGNGGANGNNNGTTGCNNATNTTTNGTCTCNTTTACCGA
ACGGNTNNTTGAGNGCTNGGCGTAATCATANGTACATATCTTGTNGCTTCGTTCTTGAAGTCANNN
ACACCACATCGAGCGGCCGCCCGGGCAGGTACAAAAGCCAANATGCCCATTGTGGGCCTGGGCAC
TTGGAGGTCTCTTCTCGGCAAAGTGAAAGAAGCGGTGAAGGTGGCCATTGATGCAGAATATCGCC
ACATTGACTGTGCCTATTTCTATGAGAATCAACATGAGGTGGGAGAAGCCATCCAAGAGAANATC
CAAGAGAAGGCTGTGATGCGGGAGGACCTGTTCATCGTCAGCAAGGTGTGGCCC

| | |
|---|---|
| Sequence 871 | cMhvSB095b05a2 |

GCNNATTGGAGCTCCCCGCGGTGGCGGCCGAGGTACGGGTCCTCCTCACGAGCTGCCGCCGCACT
GCACCGCACAGTGAAACACTGCAGGTTGTTACTGAGGAGGAAGACACAGGCTGCTGAGCAAAGTG
AGGCCAAGAACCAACATACCCACAGCAGGGAGGGTTTCACAGGCAAACAGGGCAATGGGCAGGG
GTGACAGTCAAGTATTTGTCAAATATTGCCAAGTTAAACTGCTTCTCAATAAGAGGAATGCCTCAG
AATCCCTGTGGTGTGTTTTTAAAAATATACAACTGGTCCCCATAACACCCCTAGTGAATCGCAATC
TCTAGGGGCTGAATCTGGACGTGT

| | |
|---|---|
| Sequence 872 | cMhvSB095b08a2 |

ACGCGGGGGAAAGTGTGTAGCACCTCCACCTTCTCTCTCTCTCTCCCTCTCCCTCTCCTGCCAGCCA
AGTGAAGACATGCTTACTTCCCCTTCACCTTCCTTCATGATGTTACCATTGGAATGACATACTGCAT
CCTATAGTTATACCATCCACTCTGAAATCAATGTGAATTTAACTTCAGTTCCATACAGAAACTTTTT
TTCCACAGGAGTTTAAGCCCAAGCTGGAGTGCGATGGTGCAATCCCAACTCACTGCAACCTCTGCC
TCCCAGGTTCAAGCTATTTTCCTGGCTTAACCTCCGGAGTAGCTGGAATTACAGATGTGCGCCCCC
ATGACCAGTAAGAAACGGTTGAACTGGATGCAATTTTTATCACAGCTTGTGTAAGACTGCCTCTGT
CCCTCCTCTCACATGCCATTGGTT

| | |
|---|---|
| Sequence 873 | cMhvSB095d09a2 |

AGGTACGGGTCCTCCTCACGAGCTGCCGCCGCACTGCACCGCACAGTGAAACACTGCAGGTTGTTA
CTGAGGAGGAAGACACAGGCTGCTGAGCAAAGTGAGGCCAAGAACCAACATACCCACAGCAGGG
AGGGTTTCACAGGCAAACAGGGCAATGGGCAGGGGTGACAGTCAAGTATTTGTCAAATATTGCCA
AGTTAAACTGCTTCTCAATAAGAGGAATGCCTCAGAATCCCTGTGGTGTGTTTTTAAAAATATACA
ACTGGTCCCCATAACACCCCTAGTGAATCGCAATCTCTAGGGGCTGAATCTGGACGTGTACCTGCC
CG

| | |
|---|---|
| Sequence 874 | cMhvSB095h08a2 |

NATTGGAGCTCCCCGCGGTGGCGGCCGGCCATGCTCTCCTCCTCTGCCAGTCTCCTCCACCACTCTC
TAACCTGAGAGCCTGTGGAACCTGCCCGTCTCCCCTCCTCCATCAGACACACCTGCCTAGGAAACA
GGAAAGGACCTCGGAAGTCTTCTAAGGAGAGTCATGGCGTATTACCAGGAGCCTTCAGTGGAGAC
CTCCATCATCAAGTTCAAAGACCAGGACTTTACCACCTTGCGGGATCACTGCCTGAGCATGGGCCG
GACGTTTAAGGATGAGACATTCCCTGCAGCAGATTCTTCCATAGGCCAGAAGCTGCTCCAGGAAA
AACGCCTCTCCAATGTGATATGGAAGCGGCCACAGGATCTACCAGGGGGTCCTCCTCACTTCATCC
TGGATGATATAAGCAGATTTGACATCCAACAAGGAGGCGCAGCTGACTGCTGGTTCCTGGCAG

| | |
|---|---|
| Sequence 875 | cMhvSB095h12a2 |

GAGACTTTGCCTTTTCAACATGGATGGTTCCTCCCGCTGCCGNTGCCGTTCCAGGAGACAGCATTA
CAGAGCATCAGTTAGGTGCAGAGACTGGGCAGTGCGCCCGTGTGCAAAGACAGGAGACACGAATC
TTCCTGAAGGAGTGACAGTCTAGGGAGGAAGGCAGACTGCAGGGGACCTACTTCTCTCGGGAATC
TCAATACTTGGAACAAGAACCTCCTAGACGGACCCTTTGGCATAATGAATTGGACCAACTGTAGGT
TCCAGGACTAGAGAGCCAGCAATGCCTCCATGAACAATCTCACCCAATTACTCTGCTCANGAAAC
GAGGTAACTGATGGACAGCCGAGGCAGCCCCTTAGGCGGCTTAGGCCTCCCCTGTGGAGCATCCC
TGAGGCGGACTCCGGCCAGCCCG

| | |
|---|---|
| Sequence 876 | cMhvSB096d04a2 |

CGATGTACTGNNGGTTCTNANTCAAGGATCCCAGAGNTGCCAGGAACCATCATTCATCTNCGCCTC
CTTGNTGGATGNCAAATCTNCTNATATNATCCACGATNAANTNAGGAGGACCCCCNGCTAGATCC
TGTGNNCGNTNTCATATNACATTGGAGAGGCGTTTTTCCTGGAGCAGCTTCTGGCCTATGGAAGAA
TCTGCTGCAGGGAATGTCTCATCCTTAAACGTCCGGCCCATGCTCAGGCAGTGATCCCGCAAGGTG
GTAAAGTCCTGGTCTTTGAACTTGATGATGGAGGTCTCCACTGAAGGCTCCTGGTAATACGCCATG
ACTCTCCTTAGAAGACTTCCGAGGTCCT

| | |
|---|---|
| Sequence 877 | cMhvSB096e07a2 |

GCCATGCTCTCCTCCTCTGCCAGTCTCCTCCACCACTCTCTAACCTGAGAGCCTGTGGAACCTGCCC
GTCTCCCCTCCTCCATCAGACACACCTGCCTAGGAAACAGGAAAGGACCTCGGAAGTCTTCTAAGG
AGAGTCATGGCGTATTACCAGGAGCCTTCAGTGGAGACCTCCATCATCGAGTTCAAAGACCAGGA
CTTTACCACCTTGCGGATCACTGCCTGAGCATGGGCCGGACGTTTAAGGATGAGACATTCCCTGCA
GCAGATTCTTCCATAGGCCAGAAGCTGCTCCAGGAAAAACGCCTCTCCAATGTGATATGGAAGCG
GCCACAGGATCTACCAGGGGGTCCTCCTCACTTCATCCTGGATGATATAAGCAGATTTGACATCCA
ACAAGGAGGCGCAGCTGACTGCTGGTTCCTGGCAGCACTGG

| | |
|---|---|
| Sequence 878 | cMhvSB096h06a2 |

NNATTGGAGCTCCCCGCGGTGGCGGCCGAGGTACGCGGGGAGATGATTTAGGGTCTCTGAGAGAA
GAAATTTTTAAGGATTCAAGAGGTGATCTGGCTTTTGTGAAAGTGTACGCGGGGACGGCGTCTGCT
GGCGGCCGCGGAGACGCAGAGTCTTGAGCAGCGCGGCAGGCACCATGTTCCTGACTGCGCTCCTC
TGGCGCGGCCGCATTCC

| | |
|---|---|
| Sequence 879 | cMhvSB097a09a2 |

AGGTACTGTGGGTTCTGAGTCAAGGATCCCAGTGCTGCCAGGAACCAGCAGTCAGCTGCGCCTCCT
TGTTGGATGTCAAATCTGCTTATATCATCCAGGATGAAGTGAGGAGGACCCCCTGGTAGATCCTGT
GGCCGCTTCCATATCACATTGGAGAGGCGTTTTTCCTGGAGCAGCTTCTGGCCTATGGAAGAATCT
GCTGCAGGGAATGTCTCATCCTTAAACGTCCGGCCCATGCTCAGGCAGTGATCCCGCAAGGTGGTA
AAGTCCTGGTCTTTGAACTTGATGATGGAGGTCTCCACTGAAGGCTCCTGGTAATACGCCATGACT
CTCCTTAGAAGACTTCCGAGGTCCTTTCCTGTTTCCTAGGCAGGTGTGTCTGATGGAGGAGGGGAG
ACGGGCAGGTTC

| | |
|---|---|
| Sequence 880 | cMhvSB097b12a2 |

AGGTACTGTGGGTTCTGAGTCAAGGATCCCAGTGCTGCCAGGAACCAGCAGTCAGCTGCGCCTCCT
TGTTGGATGTCAAATCTGCTTATATCATCCAGGATGAAGTGAGGAGGACCCCCTGGTAGATCCTGT
GGCCGCTTCCATATCACATTGGAGAGGCGTTTTTCCTGGAGCAGCTTCTGGCCTATGGAAGAATCT
GCTGCAGGGAATGTCTCATCTTAAACGTCCGGCCCATGCTCAGGCAGTGATCCCGCAAGGTGGTAA
AGTCCTGGTCTTTGAACTTGATGATGGAGGTCTCCACTGAAGGCTCCTGGTAATACGCCATGACTC
TCCTTAGAAGACTTCCGAGGTCCTTTCCTGTTTCCTAGGCAGGTGTGTCTGATGGAGGAGGGGAGA
CGGGCAGGTTCCA

| | |
|---|---|
| Sequence 881 | cMhvSB097c01a2 |

CCGGGCAGGTACCTGACTTTGGGTAAGTTGCCAGAATAAGATCATCAGGCTTGGCTTGGAAATTAC
ATACTTTTTCCCACCATTCTTTTGATAATATCAACGTAGGGACTCCATCTACTTCCATGATGTTAAA
CAGTTCTGGCTTTTTTTCCATCGTGGGAGCGTTTTTCTCAATCTTCGCCATTGGGAATCAGTTGGGC
TTTTGGCTTCTCTCTCCCTGTGTGAGCCAGTAAAGGGGATAATAAGGATCATTGTTTATATTCTCTG
TGAATTTATAATTAATGAAAAAGGATTTTTGTTGATCTTAAGCTGTAGACAATTTGGTGTGCTTTGC
ATGTCTTTCTGTATGGTTCTGGTATCTCAGGCAGCAGAGGAAGCAGCTTGCTGCCTTTAGTCAAAC
TGCTTCCTGGAAAC

| | |
|---|---|
| Sequence 882 | cMhvSB097c02a2 |

GGCNAATTGGAGCTCCCCGCGGTGGCGGCCGAGGTACTGTGGGTTCTGAGTCAAGGATCCCAGTG
CTGCCAGGAACCAGCAGTCAGCTGCGCCTCCTTGTTGGATGTCAAATCTGCTTATATCATCCAGGA
TGAAGTGAGGAGGACCCCCTGGTAGATCCTGTGGCCGCTTCCATATCACATTGGAGAGGCGTTTTT
CCTGGAGCAGCTTCTGGCCTATGGAANAATCTGCTGCAGGGAATGTCTCATCCTTAAACGTCCGGC
CCATGCTCAGGCAGTGATCCCGCAAGGTGGTAAAGTCCTGGTCTTTGAACTTGATGATGGAGGTCT
CCACTGAAGGCTCCTGGTAATACGCCATGACTCTCCTTAGAAGACTTCCGAGGTCCTTTCCTGTTTC
CTAGGCAGGTGTGTCTGATGGAGGAGGGGAGACGGGCAGGTT

| | |
|---|---|
| Sequence 883 | cMhvSB097g01a2 |

CGAGGTACTGTGGGTTCTGAGTCAAGGATCCCAGTGCTGCCAGGAACCAGCAGTCAGCTGCGCCT
CCTTGTTGGATGTCAAATCTGCTTATATCATCCAGGATGAAGTGAGGAGGACCCCCTGGTAGATCC
TGTGGCCGCTTCCATATCACATTGGAGAGGCGTTTTTCCTGGAGCAGCTTCTGGCCTATGGAAGAA
TCTGCTGCAGGGAATGTCTCATCCTTAAACGTCCGGCCCATGCTCAGGCAGTGATCCCGCAAGGTG
GTAAAGTCCTGGTCTTTGAACTTGATGATGGAGGTCTCCACTGAAGGCTCCTGGTAATACGCCATG
ACTCTCCTTAGAAGACTTCCGAGGTCCTTTCCTGTTTCCTAGGCAGGTGTGTCTGATGGAGGAGGG
GA

| | |
|---|---|
| Sequence 884 | cMhvSB101h01a2 |

NGATTGGAGCTCCCCGCGGTGGCGGCCGAGGTACCTGACTTTGGGTAAGTTGCCAGAATAAGATC
ATCAGGCTTGGCTTGGAAATTACATACTTTTTCCCACCATTCTTTTGATAATATCAACGTAGGGACT
CCATCTACTTCCATGATGTTAAACAGTTCTGGCTTTTTTTCCATCGTGGGAGCGTTTTTCTCAATCTT
CGCCATTGGGAATCAGTTGGGCTTTTGGCTTCCCTCTCCCTGTGTGAGCCAGTAAAGGGGATAATA
AGGATCATTGTTTATATTCTCTGTGAATTTATAATTAATGAAAAAGGATTTTTGTTGATCTTAAGCT
GTAGACAATTTGGTGTGCTTTGCATGTCTTTCTGTATGGTTCTGGTATCTCAGGCAGCAGAGGAAG
CAGCTTGCTGCCTTTAGTCAAACTGCTTCCTGGAAACCCAGAACCAGGTCCAGCTCCAGGACACTG
TGCAA

| | |
|---|---|
| Sequence 885 | cMhvSB105g08a2 |

GCCATGCTCTCCTCCTCTGCCAGTCTCCTCCACCACTCTCTAACCTGAGAGCCTGTGGAACCTGCCC
GTCTCCCCTCCTCCATCAGACACACCTGCCTAGGAAACAGGAAAGGACCTCGGAAGTCTTCTAAGG
AGAGTCATGGCGTATTACCAGGAGCCTTCAGTGGAGACCTCCATCATCAAGTTCAAAGACCAGGA
CTTTACCACCTTGCGGGATCACTGCCTGAGCATGGGCCGGACGTTTAAGGATGAGACATTCCCTGC
AGCAGATTCTTCCATAGGCCAGAAGCTGCTCCAGGAAAAACGCCTCTCCAATGTGATATGGAAGC
GGCCACAGGATCTACCAGGGGGTCCTCCTCACTTCATCCTGGATGATATAAGCAGATTTGACATCC
AACAAGGAGGCGCAGCTGACTGCTGGTTCCTGGCAGCACTGG

| | |
|---|---|
| Sequence 886 | cMhvSB105h02a2 |

ATTGGAGCTCCCCGCGGTGGCGGCCCGAGGTACTGTGNNTTNTTATTNNTNGATNCNATTGCTGNC
ANGAACCAANATTNATNTNCGCCTCCTTGTTGGATGTCAAATCTGCTTATATCATCCAGGATGAAG
TGAGGAGGACCCCCTGGTANATCCTGTGGCCGCTTCCATATCACATNGGAGAGGCGTTTTTCCTGG
ANCAGCTTNTNTCCTATGGAAAAATCTGCTGCNGGGAATGTCTCATCCTTAAACGTCCGGCCCATG
CTCAAGCANTGATCCCGCAAGGTGGTAAAGTCCTGGTCTTTGAACTTGATGATGGAGGTCTCCACT
GAANGCTCCTGGGTAATACNCCNTGACTCTANNTAAAANACTTCCAGGTCCTTTCC

| | |
|---|---|
| Sequence 887 | cMhvSB024b11a2 |

CCCTTAGCGTGGTCGCGGCCGAGGTACGCGGGGAGCTATATCGGGGATCCAAAGGTTTCACACAG
GATGAGTCCTGTGTCTACATGCAGCGTAGCAGGAGCTGGGAATGGAAGCAAACCAATATTCCAGC
ATCTGCTTCTAGAACAGTGATCAGGATCGCTATCGTTAATAAGATGGGTGTATGTGGGACCCAAGA
CTCATCTGTCAAGCCCTTCTTCTGACTGCTTTTAAGGTGCCAGTCACGAATTGCCCGAACATTACCT
GCTGATCAGAACCAGAATGTCGGCCATACTGGGAAAAGGATGATGCTTCGATGCCTCTGCCGTTTG
ACCTCACAGACATCGTTTCAGAACTCAGAGGTCAGCTTCTGGAAGCAAAACCCTAGAAGGAGCAC
AAGTC

| | |
|---|---|
| Sequence 888 | cMhvSB038e02a2 |

ACTGATTGGGGAAGTGATAAATGTTCATGAAATCTTCACAATTTATGTTCAGAGATTGCAGTAAAG
ACAGGCGTAAGAAATTATAAAAATATTAATGTGGGGAATTAAGAAATGTCCATGAAATCTTCACA
ATTTATGTTCTTCTGCCATGGCTTCAGCCAGTCTCTCTGTTGGGGGTCCCTGAATTCCTGCAACAGC
TCAGAAACTAGAGGCTGAGAAAGGGAGTCACTCAAACCTTGAATCCCTGTGGCCAGTGAATAAGA
TAGACGTCCAGATAGCTCAGCTTCAGGTCCTTGAGGGTCTTCTCAAAGGCTTTCCTCACAAGGGGT
CTCTCAAAGAAAGTGGGCCA

| | |
|---|---|
| Sequence 889 | cMhvSB101b12a2 |

NTTCTNNATNTATTGGNTACGCTGGTCTGGNANANTTGANCTTNAGNNNTACACNNACTNNNGAC
NTCCANGGGGNNCNNATTACCGNCATNANCCACCNTNNTGNGNNGNNNAANATNGCNNTTNNAA
CAAACATNNNAAANACTCNCCTGTGGCATTCGTTTCCTAGGGCTGCATANCAAAATACCACAAAC
TGGTTGGCTTACAACATCATTTAGTTTCCTACAGTTCTGGAGACTGGAAGTCTAGGCAGCAGGGCC
TTCTGACCTCTCTCATTGGTTTATANATGAAATGCCTCTTCTCCCTGTGTCTTTACAAGGNCTTTTCT
GTACCTTTCTATGTCCTAATCTCCTGTTCCTGTAAAGACACAGTTATATTGGATTAAGGCACATCCC
TAGTGACTTCATTTTACTTTAA

| | |
|---|---|
| Sequence 890 | cMhvSB082a07a2 |

CCGGGCAGGTACCATGTTCAGGAAACCAAGGACGATATTGCTCTACTGTTGGAAACAGAGTAATC
AAATTTTCTGTGCTAGCCTTAATTCCTGCCCTCTTTAAGAGGAGCTTAATAAAATGTAAATATGCA
GAATGTTTACTTTTGGATTGTCCCATGGTGTCCCTGGAATGCTCCGAGTGCACAAGCTTACCGCAA
GGCCGACCACACGTTCTCGGGAGTTCCTGGACAGACCGTTCTTCACAACGACCACGCTCAGGTGTA
ACTTCACCTGGGTTCAAGGAGACCGTGTTGGGTGCCAAAGATGTAGGGGAACCTGCCTGATACAC
CACCCGCAGGCTCTCCCCTTCCCGGTGGAGACGAGGGAATGAGAAAAGAAATAAAGACAAAGAC
ACAAAGTTTAAGAGTTAACAAAAGTGGGTCCAAGGATCCATCGCAACGTGGAGATTGCAAAGGCC
CCCGCGTACCT

| | |
|---|---|
| Sequence 891 | cMhvSB030a07a2 |

CCCTTTCGAGCGGCCGCCCGGGCAGGTACTTTCTCTTGGTCTCTGCCATCACAATGGCAGCCCGGG
TTCGGGGTTGAATTCCCAGCTTAAGGGATCATCCTTTGTCTTCTGTTTGTCTATGTATTTATATGTA
GTATGTGTGTGTAATATAAAAGAATTTTAATTAATTGCTTTAATAATAATAAGCTTAAATCAAATA
TTTTGTCACATAAGTAAAAAGTGTAATGCCTTTTAGTTCATGTGACTTAAGTAATCTTTGGGAAATA
AAAACAGTTTTAAAGATTACTGGTAAAATAAAGACATTTGGTCTAAATTATGCAGGTCAGATATTA
AGTTTGCTAAATGCCTTAAGGTCATAAACTGCTGCTTTGACTTTTTTTTTTTTNGAAANAAAACCNC
CCCCNGGGNNACAGANNNAAATTTCATNTCCTNTNANTAAATAATTAACCCCTTTTTAAAAAGTCC
AAAANCCCNCAAAAGTCCAAAACTTAAAAANTTTNAACACTGGACCCNAGGCCNAAGNTAAAAC
NTTTTNCNTTTTAAACCTCCTTGGGNATNGGNNCNCCANTNAAAAANGCNNGGGAAAAACTTTGTT
TTTTTCCCNAAAAANTTTTTTAAAAATTTTTNGTAAAAATTGCCCTTTTNGGGTTTTTTTTNGTNAA
GGGNGTNTTTGNAAANAAAAATAAAATTTAAAAGNTTGGCCCCNTTGGGGGNTTTTNCCCCNNGG
AATTNNNNNAATTTTGTTNGCCAAAANTTTCCNAAAAAAAAAAAAAAA

| | |
|---|---|
| Sequence 892 | cMhvSB095f04a2 |

ATTGGAGCTCCCCGCGGTGGCGGCCGCCCGGNCAGGTACGCGGGGGAGTTCTGCTCTGTACTTTGC
CCACTTGGGTTCTATTCTTATCTCCTCTTAGCTTTGGCTCTCCAGCATGGACTTTGCTTGAGTCTTTG
ATCTTGCATCAACTGATGTTTCTAGTAAGGGCCGACACCACCTCTCTCCCAGTGCTGACAGATGAC
ATCCCTGCTGAGTCCCGATTTCCACCAGCTGTTTAGCGTTCTGGATCATTCCCTGTTGACCAGCTGC
TTCTGGCCATCCTCACCTGGACAATCTGCAGTAGTTTTGGCATGTTGCTCACTGCTTCCATTGGCTG
ACGGTTTGAAGAAGAACTGACCAGCAAGTGGTTATATCTTTTTGAAGGCAGTGGAGTCCCGTATGG
CCCAATCAACAACATGAAGAATGTATTTGCAGAACCTCAGGTATTACACAATGGCCT

| | |
|---|---|
| Sequence 893 | cMhvSB031h07a2 |

CCCTTTCNAGCGGCCGCCCNGGCNGGNACAGTGCNTCCCAAAGCCCCCAGANGCCTACCCCTGTC
GCCNGTGTGCCCACAATGAAGAATATACAGTCAAGGAAGATGATTTTGCAGCTCTAAGATNTAAT
TTCTGCCCTGTNATCTTTATGACTTGCATGAACCCTCTTGCTCTTCTCTTTAAGCTGANATTTCCCT

| | |
|---|---|
| Sequence 894 | cMhvSB013d11a1 |

CTACATAAATGGGGGTTTCACAGTTCCGTTCTACAAGCAGCTCCTGNGGAAGCCAATCCAGCTGTC
GGACCTGGAGTCCGTGGACCCAGAACTGCATAAGAGCTTGGTGTGGATTCTAGAGAATGACATCA
CGCCTCCCGCGTGGCGGGCTGAGGCCTGAGATTCCAGAAACCGAGGGAAAAGGCTCGTCTCCCTC
CTCCTTTGGAGAGGGCAGGCCAGGGGACTTTCCTAGGTGGCTCCCACCCATTTATTCTCCTTTATTA
TAGTTTGCCCACCCCTCCATCACCCATCCAATAAAACGCAGCCAGGTTTCGCCCTCAGNAAAAAAA
ANTTTNACAAAAATNNGGGNANAACNNAANAANNAACCTNTNNCCAAANGNCCNNTTAAANGGC
CNNAANCNCNAAANNGGCCCNNNNGGGGGNGGCCGTTAAATTTTTNAAAAAAAAAACNTTCNAC
ACCCTCCCTTGANCNTGAANAAAAAAAGGANNGCACCTGGGGGGGGNAACTTGTTTTTGGCCCTT
TTAAANGGTTCNAANTNANNCNATGCTTTNCAAATTTCCCAAAAAAAGCATTTTTTTNCCCGGGNT
TTTNNTNGGGGGTTGGCCCAACCCANNNNNGGTTTTTTNTNNTGGNTGGNACCCCCG

| | |
|---|---|
| Sequence 895 | cMhvSB093a04a2 |

CTGATTGGAGCTCCCCGCGGTGGCGGCCGAGGTACCCTTTCTGCAGAAAGTATAAAAATGGCCTTG
CTAAGGAATTTAAATTTACATTCAAGTGCTATTTCTTTACAGCACCGGAAAACAAGCATTTCAAAC
AAGACCTACTATACAATGACAGTAATTAAGATAATGTGATACTGGTGGAGGAATAAGCACGTAGA
CAAATCGAACATAATAGAGAACCCAGAAATAAACCCCTACAAATATATACGCAACTATTTTTTAA
CAAAGATTCAAAAGCAATTCAGTGGAGAAAAAATGACCTTTTCAACAAATAATGGTGGAGCANTT
GAACATCTACAGCAAAAACAAAGCTCAACTTCAACCTCACACCTGATATAAAACATGAATAAAAA
ACTATGAAACTTTTAGAAAAATAAATAAATAAACCTTAGG

| | |
|---|---|
| Sequence 896 | cMhvSB038c05a2 |

CCCTTAGCGTGGTCGCGGCCGAGGTACTTTTTTTTTTTTTTTTTTTTTTTTTTTTTTTTTTTTTTTTTTT
TTTTTTTTTTTTTTTTTTTTTTTTNCCNCCNANNNNNNAAAAAAAAAAANNTTTNGGGGNGGAAAA
AAANTTTTTTNNTTTTNNNGGNAAAAAAAAAAAAAAAAAAANGGGGGGGNNNNNAAAAAAAAAA
NNNNNNNTTTTNNNNNNNNNNCCCCTTTTTTTNAAAAAAAANNNTTTTTNNAAAAAAAAAAAANA
NCCNCAAAAAANNGGNTTTTTTTTTTTNNNAAAAANNCCCCCNAATTTTTNAAAANTTTNNNNGGG
GGGNAAAAAAAAAAAANNTCCTTTTTTNANNTGGNTTTNNGGGNNNAGGGTTNNCNNTTTTNCCC
CNNCAAAAAAAAAANNTTNTTTTTTTAAAANAAAANNNACCCNNTNTNTTTGGTTTTTTNANAAA
AAANCCNNNNCCCCAAAGAGGGGGGGGGGNGCCNNNCCNCTTCNTTTTTNTTNTTTTNGGNNNNG
ANGGGNNNNCNCCAAAAAAAAAAAANAAAAAAAAAAANNTTTTTTTTTGGGGGGGGGNTCTTTTT
TTTTTTNATANNNANNNCGGNGGGNNNAAAAAAAAAAAAAAAAAAAANT

| | |
|---|---|
| Sequence 897 | cMhvSB083h11a2 |

AGGTACATTCTCACGACCGGCCTGATCCCTGTGCTGGAGAAAGAACACGACCCCCGAGTGATAAC
CGTCTCCTCAGGAGGAATGTTGGTTCAGAAACTGAACACCAATGATCTCCAGTCCGAAAGAACAC
CATTTGATGGAACTATGGTCTATGCACAAAACAAGAGGCAGCAAGTGGTTCTGACGGAGCGGTGG
GCCCAAGGGCACCCGGCCATCCATTTTTCTTCCATGCATCCTGGCTGGGCCGACACCCCAGGTCAG
ACAGGAATGAGCAGGAGCTGAGGAAGGTAGTGGGAGAGGCCCAGACTGCCTCACCACTCCCCAG
GTTTTTGGAAATAATGATGCATGAAGGTAAATGCCAGCCACAAGGACACAGCTCGAATGATCTGG
AAGCGTGTTGGAGCAGCGGTGGAGGGGAGCAGAATTCTCTTCCGGATTGGCCTCACCAACTCCAT
GACCTCAGGCAGCTCACCTGGGCTCTCTGCAGCTCTTTCCTCCTCTACAAACAAGGGAACTGAAAG
CAGCAACAGCCACAGCACACACCCCAGGGTGCACCCGCGGGCGCCAAAGAACTGGTCTCAAGCGC
TTGTCTTGCGGATTAACGCATTTTGTCCTCAAGCCCTCTGTGGAGTGGNCCTACTGTCTTTTATCAC
ACCCATTTACAGATGAAGGGACTGANGCCCCAAANAGCTTAAAACTTCCAACCCGGCCTGGCCAT
GGGGTT

| | |
|---|---|
| Sequence 898 | cMhvSB092h03a2 |

CCGGGCAGGTACACTCATATGGTTTTACTCCGGCAGTCTTCTTCGTCACACTGAGATTGGGACTGA
AGTTTTCTGCACATTGACTACCTTCTTTACCTTCACAGAGTCTCTCTCCCGTATGGCTTCTTANATTT
CGTCCTTGGTTTTTGTGTTGATCTTCAACATTCGGGTCTTCCCATTTTTCCCCTATAGATGCCAGGTT
CTTGAATGTTTCCTGCATCACATCTCTGTANAGTTTCTTCTGTGAAGGAGCCAGCAGAGCCCACTCC
TCCTGGCTGAAGCTCACAGACACATCCTCAAAAGCCACTGAGTCCATTTTCCGGCCTCGCGGGTGT
CCCGGTGTTGTCCCTAAGGTTCACGGAGCCAGCGCAGGGTACCT

| | |
|---|---|
| Sequence 899 | cMhvSB097a08a2 |

ATTGGAGCTCCCCGCGGTGGCGGCCGAGGTACAAACTTGTTTCCAGGCAAACTTGTCCAACCCATG
GCCCACGGGCTGCATGAGGCCCAACACAAATTCACAAACTTTCTTAAAACATTATGAAATTTTTTT
GGTGATTTTTTTAGTTCATCAGCTATTGTTAGTGTATTTCATGTGTGGCCCAAGACAATTCCTCTTC
CAATGTGGCCCAGGGAAGCCAAAAGACTGGACACTCCTGTCCTAGAATATTTAATTTGGGTCTGCC
AGAGAGGTTAAAAGAATCGTAACTTTTTAAAAAGCCTGTAATTTTATTTTTATTTTTACTAGATATG
GGGTCTTGTTATACTAACCCAGGCTAGTCTCAAACTCTTGGCCTCAAGAAATCCTCTCACCTCGGC
CTCCCAAAATGCTGGAAATACAGGCATGAGGAACCACACCCAGCCAGCCTACAATTTTAAAACCT
AAGGCA

| | |
|---|---|
| Sequence 900 | cMhvSB032f05a1 |

CCCTTAGCGGCCGCCCGGGCAGGTACGCGGGGGCTGCTGGAAACGCAGTTCCGGTTAGGCGGCTG
AGTTTGTTTACGTTGCTAACAGATCTAGCCCCTGCTTTCCCTAGTTCCAGTTCCAAGATGGGGAAAT
CCTTCGCCAACTTCATGTGCAAGAAAGACTTTCATCCTGCCTCCAAATCCAATATCAAAAAAGTAT
GGATGGCAGAACAGAAAATATCATATGATAAAGAAGAAACAAGAAGAATTGATGCAGCAATATC
TTAAAGAACAAGAATCATATGATAATAGATTGCTTATGGGAGATGAACGTGTAAAGAATGGGCCT
TAATTTCATGTATTGAAGNCCCCCCCAGGAGCTNAAAAAAAGGA

| | |
|---|---|
| Sequence 901 | cMhvSB096b05a2 |

CCGGGCAGGTACGCGGGCGTGGGGGTGAGGGTTGAGAACCTATGAACATTCTGTAGGGGCCACTG
TCTTCTCCACGGTGCTCCCTTCAAGCCAACAAGGCCACACTGGTGTGTCTCATAAGTGACTTCTACC
CGGGAGCCGTGACAGTGGCCTGGAAGGCAGATAGCAGCCCCGTCAAGGCGGGTGTGGAGACCAC
CACACCCTCCAAACAANGCAACAACAAGTACCT

| | |
|---|---|
| Sequence 902 | cMhvSB092a05a2 |

ACTTTGGCCTCTCTGGGATAGAAGTTATTCAGCAGGCACACAACAGAGGCAGTTCCAGATTTCAAC
TGCCCATCAGATGGCGGGAAGATGAAGACAGATGGTGCAGCCACAGTTCGTTTGATTTCCACCTTG
GTCCCTTGGCCGAACGTCCACGGAGTAGTATAATATTGCT

| | |
|---|---|
| Sequence 903 | cMhvSB092a05a2 |

TCGGTCAGGGACCCCGGATTCCCGGGTAGATGCCCAGTAAATGAGCAGTTTAGGAGGCTGTCCTG
GTTTCTGCTGGT

| | |
|---|---|
| Sequence 904 | cMhvSB092a05a2 |

GCGCCCGGCAGGTGATACCTCCGCCGGTGACCCAGGGGCTCTGCGACACAAGGAAGTCTGCATGT
CTAAGTGCTAGACATGCTCAGCTTTGTGGATACGCGGGACTTTGTTGCTGCTTGCAGTAACCTTAT
GCCTAACAACATGCCAATCTTTACAAGANGTGAAGTAAAACTTTTTTTAAGAATTTTTAAAAATAC
TTTGATTCCCTTGGCTACAGGTGATGTCTTCTCTTGGAANGGGAAGAAATTACCATTAATATTGAC
CATTCCTANATTCCCA

| | |
|---|---|
| Sequence 905 | cMhvSB094f03a2 |

AGGTACTGAGGATGAATTTCATGCCACTGGCCTCCAAAAAACCCACTGGAAACATTGCACGTGGA
GTAGCTGTCTGTCCAGGCTGGCGGCTGGTGAAGGAGGTTGTTGCCGGGGTTGAGATTCATTACACC
ACCTCCTTCCAGAATCATGATCTTGAGAGGTCTTGATGAAGGCTACCATCTTGCGCAGTCATGTAA
GAGAACTTACAGCACAGCTGTTCCCTCAAAGTGACTTTCATTTAAAATGCCTCTCATTTACCTAAA
GATTCTGGGTGGGAAATCCAATAGCTGTGGCTGATGGAGGGGAGGCAGCAGGCTGCAATCTCACC
AGCTCCTATAGGGATGGGGCACCACGGGCGTTATCAAGTCTCCCCGCGTACCTGCCCG

| | |
|---|---|
| Sequence 906 | cMhvSB038d09a2 |

CCCTTAGCGTGGTCGCGGCCGAGGTACTACTGTGTGTTGACTCTTGTAAATCCTCCCAGTGAAGAG
TCATCAAACCTGGGAGTGGTCTTGGGGCCCTGACATACCACTTCATGGAGCTGGTGATGGAAATTT
GCTGATGTTGTTGGCCACCCGAATGAGCATGCGAGCCCCTTTCATGTGATCTCCATTTTTAACATGA
ATCTTTACTAGTATATAGCTGTGCAGAATCATGAGGTTGGTGGCCATCTCGGAGGGAATTTTGATC
TTCTGGGATTTCAGTTCTGCATACATACTGAAGAGAACATCGTGTGCATTCCGGTAGTTGC

| | |
|---|---|
| Sequence 907 | cMhvSB038b07a2 |

CCCTTTCGAGCGGCCGCCCGGGCAGGTACCCACTCACAGTGATGCCAGCAAGAAGAGACTGATTG
AGGATACTGAAGACTGGCGTCCAAGGACTGGAACAACTCAGTCTCGCTCTTTCCGAATCCTTGCCC
AGATCACTGGGACTGAACATTGTNAGTGAACTTNTAGGTATCCTAATGGATGAATGTTTTTTTGCC
CCAGAGAGTGGCATTGAAACTGATTGGTAGTTGTCAGAAAACAACCCCGAGACAGTTTGCTTTTAA
ATTATGCTGTGCATAACATGGGTAATATAAATAAGACCCCAGGCCGGGCACAGTGGCTCACGCCT
GTAATCCCAGCGCTTTGGGAGGCCGAGGCAGGCAGATCATGAGGTCATGAGTTCGAGACCAGACT
ANCCAACATGGTGAAACCCCGCCTTTACTAAAAATCAAAAATTATTTGGGCATCGTGGAAACCCCT
GTAATCCCANCTNTTTGGGAGCCTTGANGCAGGANAATCATTTTGAA

| | |
|---|---|
| Sequence 908 | cMhvSB042b12a1 |

CACCGNGGTGGCGGCCGAGGTACTTTTTTTTTTTTTTTTTTTTTTTTTTTTTTCGNGCTCCANCAATT
TTTNTTTTCATGAATGAAAGTTGGGGATCAGCTGTTAGGTTCTGTGCCCAGNACACTGANTGNTGC
CTGGCNCCCACTTTTTATACAGTCNTTAACAGCAACTCCNTCATAGGAGGCTCCAGCCANAGTCAG
GGGCAACCTGTGAGCAGTCAGGAATTGCCTAGCTGACTNTAGTTTTTGCCAGTGGACCCTAGNGTA
TACTGGGGAATGCAGTTNTTGTGTAGATGGACCAAGNCAGTTGGCTCGGCNTNTCCTTAAANTCCT
AAATTTGGNGTAAGCAAGCTGNTTCNCTGGGCCCCGNTTGTTTGAAAACAANNTTCNCTGGANAA
TAANACACAAGCCCACTNAGCCCTNCAGGTGGTCCTGGTAACCAGGAAAACCNTCCCCANGCCAT
CACNAGTTCACNTTNTTNGAGGGGCCCAGGGGG

| | |
|---|---|
| Sequence 909 | cMhvSB079f12a2 |

GATTGGAGCTCCCCGCGGTGGCCTGGTTAGCAAATGTTTCTTCCTCCCTCACAGGCTATAAGAGCA
ATGAGCTGGCAACGCCCCTGANCACACTGTCTGCTGGTTAACCAATGGCATGTGAGAGGAGGGAC
AGAGGCAGNCTTACACAAGCTGTGATAAAAATTGCATCCAGTTCAACCGTTTCTTACCTGTGGAAA
AGAAGTTTCTGTATGGAACTGAAGTTAAAGTCACATTGATTTCAGAGTGGATGGTATAACTATNGG
ATGCANTATGTCATTCCAATGGTAACATCATGAAGGAAGGNGAAGGGGAAGTAAGCATGTCTTCA
CTTGGCTGGCAGGANAGGGANAGGGAGAGAGAGAGAAGGTGGAGGTGCTACACACTTTCAAACA
ACCAGATCTCATGANAATTCTATTATGAGCCCCGCGTACCTN

| | |
|---|---|
| Sequence 910 | cMhvSB051c06a1 |

AGGTACACGCTGGGGGACGCTCCTGACTATGACAGAAGCCAGTGGCTGAATGAAGAATTCAAGCT
GGGCCTGGACTTTCTCAATCTGCCCTACTTGATTGATGGGGCTCACAAGATCACCCAGAGCAATGC
CATCCTGCGCTACATTGCCCGCAAGCACAACCTGTGTGGGGAGACAGAAGAGGAGAAGATTCGTG
TGGACATTTTGGAGAACCAGGTTATGGATAACCACATGGAGCTGGTCAAGACTGTGCTATGACCC
AGATTTTTTGAGAAACCTGAAGCCAAAATACTTGGAGGGAACTCCCTGGAAAAAGCTAAAGCNCT
TACTCAAGAGNTTTCTGGGNGAAGCGGGCCATGGTTTGCAGGAAGACAAGGATCACCTTTTGTGG
GATTTCCTTGCCCTATGAATGTCCTTTGGACATGAAAGCCGTTATTATTTTTGAGCCCCAAGNTGGC
TTTGGAACCGCCCTTTCC

| | |
|---|---|
| Sequence 911 | cMhvSB079b08a2 |

ATTGGAGCTCCACCGCGGTGGCAGCGGCCGCCCGGGCAGGTACCACTTCTGCCCTCAGATGGTTTG
AACTCTCCTAAGCCAAGAGGCTGGAATGACTGAGTTGTCCAAACAGCAAAGATGGTGGCTCGTCC
CTACCCCTCGGCACTCCATCCCAAGGAGAAATCAAAACTCTGTCTGCCAGAGAATATGGGTGGGG
TTGGCTGGAGGCCTTGGTTGGGAGGCCCTGCCCTAAGATGAGGAATGGATCAGGTCCCACTTAAA
GAAGCAGTCTGGCCATGTTTTGGTAGAACAGCTGTGCTGTGCTGGGAGGTCCCATCAGTTCTCANT
TGGTGTGGTTTGGACTCTCCTACACCCACATGCTGGAATGGCTGAGTTGTCCAAACAGAAAAGATA
GCGGCTTGCTCCTTCCC

| | |
|---|---|
| Sequence 912 | cMhvSB068b04a1 |

CACAAGGTGCATTCTGCTTCCTGCAGGGGCTTGAAACACCAAGGCACTCCAGGGATCCTGGAGTC
AAAGCAGCAGCCCCGGTATGTTGCACTCCTTGGGGGTGACATGGGGGTAGCCGCAGTCCACCCTG
TCCTTGGCTGGCACGGCACACTGGTTTGCAGACAGGCCCACGTACTCCTCAGCAGAGCTGGAGGG
ACAAGCAAGGCCAGGACCAGCCCCAGCNATGCCAGAGCGCTCTGGCAGCCATGACCACNCGTTGG
GGNCTCCCGGGGACGCCAAGCTCAGGACTCCCGCGTACCTTGCCCCGGGGCCGGCCCGCTCTTAG
AAACTAGGNGGGATCCCCCCGGGCTTGCAAGGGAAATNCCGATATTCAAAAGCTTTATCCGATTA
CCCGTCNGACCCTCCGNAGGGGGGGGGGCCCNGGNTACCCCAAGCNTTTTTGTTTCCCCTTTTAAG
GTGGAGGGGTTTAAATTTGGCCGCCGCCTTTGGCCGGTAAATTCAATGGGTCCAATAANCTTGTTT
TCCCTTGTTGTTGAAAAAATTTGNTTATTCCCGCTTCAACAAATTTTCCAACAACCAACAATTACCG
AAGCC

| | |
|---|---|
| Sequence 913 | cMhvSB092a07a2 |

CNNATTGGAGCTCCACCGCGGTGGCCGAGCGGCCGCCCGGGCAGGTACTTTTGTATGACACTAGA
CTTCTGCTGTAGTGCTTCACCCAAAACAGAGGTTTAAGGAAATAAAAAAATAAAAATAATACAGA
AAAAAAACCAAAACACTTTACTGAAAATTTTCATTTCAACCAGAAGCAAACGTGTTCTAAGAAGG
CAAAGTAGAGTTAGGAACAACTCCGTGTTTCCCTCAGGAATAAACGTGATCTTTCACACTTGGGGG
TTGATAGTCAGCATGGAGTAACTTAGACCAACTTAAGAAGGAGGCATCTGGGGCTGTTCACCTAA
GGAGATGCTTCCCAGAGGCCCAGCATCTTGGGAGAACACCCCAAGTTCTCTGGAGAGGTCAGGAG
TTTGGGATGCAGGATCACACTGAAGGTCAGCCCAGCAAAGCAGCTGATCTAGGATATGGGCTTCT
GACTTCCAGATTCTACCATCATCACAGAGGCTCAAAGCTGGGGCCCACACCAAAAGGGCGTGATG
ATTCCCAGCCTTCAGCACAACAGGAATTGACCTGGAAAGAAAGGCCTTTATTCCTCTGACAGAAA
AACCTGATTCCCAAANGAAAATGATACTTTTACCTTATTCCCTTTCTCAATGGATCTGCATTTTCAT
GAATGAAGAAAAGAAGAAAGTTGAATTCTCTGACTTAGGAANGTTTCTTATTAAAAAGGTTNCAA
TANACTTCAACTTTTTTNAAGCTGGGCAGCAAAAAAAAAAAAAAAAAA

| | |
|---|---|
| Sequence 914 | cMhvSB068c08a1 |

GAATTGGACTCCACCGCGGTGGCGGTACAGCTTGGAGTGATCCCCCACGGTTTCAATTTTAAACCT
CTCATCATCTGAAATCTCCTCGTAGGATTTACACCAGGTGAACTGAGACGCGTCTGTCATTTCCTG
GCAGTCGAAGCCCAGATAGATGTTGCCTTGTTCATCGACACCAGCACTGATTTCCTTGGTGCCTGG
TCTCGCCTCTACCAACACAGGCTCCGACGTGTCTGAGGGCTTCCCCACGCCATTTGCATTGACTGC
CCGGACCCTGAAGACATAGGTCTTACCTTGCTGCAGGTCAGAAGACCTTTAAAATAACGGTTTGGC
TGTTGGTCGTCCTGATTGACAGGTGATCCACTCCTCCAGCCATTCCCTCCTCCCTGGAAGTCCACCG
AAATATCCAGAAAACAGGGCTTGCTGCCGGGAGNTACCTCCGGCCGCTCTAAGAACTAAGTGGGA
TCCCCCGGGCTGCAGGGAATTCGATTATTCAAGCTTATCGATACCGGTCCGACCTTCGAAGGGGGG
GGGCCCCGGTACCCCAAGCTTTTGGTTCCCTTTTAGTGGAGG

| | |
|---|---|
| Sequence 915 | cMhvSB026c05a2 |

AGGCTAAGGGAGGCTATGGGAGGCTAAGGGAGGCTCAGGTAAGGAGGATCTCTTGAGCCTGGGA
GGCAGAAGCTGCAGTGAGCCAAAATGGCACCACTGCACTCCAGCCTGAGTAACAGAGTAAGACTC
TGTCTCAAAAAAAGAAAAGAAAAGAAAAAAAGAATTCAAAGGAGAACTGACATATCACCCAGTG
GGTATATTACAGAATGCTTGCATGTATGTGTGTGTGTGTATGGTTTTATATATATTTATATAAAGTA
TAAATGCTTTTGCTTATATATATGAATCTCATTTTCCCACTGGCTTTCCTTAAAAACTAAACAAAAC
ACAAACACCTTACTGATCTTTAGTAGCTCGTAAGCTGATTTTTAGCCTTTCAGCTGAGAGGAAATG
GTCCAAAAAAAAAAAAAAAAAA

| | |
|---|---|
| Sequence 916 | cMhvSB096f10a2 |

TGATTGGAGCTCCCCGCGGTGGCGGCCGAGGTACATGCATTGGGATTCATCAAGGAAACAAAGCT
GGACCAAAGATGGCTGACTAGAAGCAGTGAGGACTCTTGGCTCTCATGGAGAGAAATGAAAGGG
GCAAGTAAATACAGCAACTTCAACTGAAACATTCATGTTCTCACATTGAGACTGATCAGGGAAAG
AGCTCAACCCATGCAGAAAGGAGAAAAGCAAAGCAGGGCGACAGCCCACTAGGAAGGACATGGA
GCCAAGGGAACCTCTCCCTGCCCAGGCAAACAGTGAATGAATATGTGACCCCTAGCAACCGCACT
TCTTCCATGGACCTTTGCAACTCTTGGGTCAGGAGATCCCCTCATGAATCCACTCCACCAAGACTT
GGTCTGACACACAAAGCTGCATGAAGTCTCTGCTAAGCAACTGCCCAGGGGTGCACAGAGTCCCA
GGAGCTTTACATACTCTGGCCCCAGGATCCCTG

| | |
|---|---|
| Sequence 917 | cMhvSB027a02a2 |

CCCTTAGCGTGGTCGCGGCCGAGGTACTTTTTTTTTTTTTTTTTTTTTTTTNGGGGGNNGGNNNAAA
AAAAANTTTANNNTGGGGAAAAANNNCNAAAAAAACCCCCCANNANNTNNNTTTTTTTTTNAAAA
AAAAANNNNAANNTTTNNNTTTTTTTTNAAAAAAATTTTNCCCCCNGGGGGGNTTAANCCCCAAA
ANTNAANNNNCCNNTTNGNAAANCCCANGGNTTTTTNTTTTTTTNCCCCNNAAATTNNNNNNNCN
NGGNNNGNCNTTTAAAATTTTTTTTTTTTAAAAANNNAAAAANTTNNCCNAAAGGGNTTTTNCCAA
NNNNNNAAAAAAANGGGNTTTTTTTAAAAAAAAANCNTTTTTTTTTTTTTTAAAAAAAAAA

| | |
|---|---|
| Sequence 918 | cMhvSB091a12a2 |

ANCCNCAAAAAAAAAAAAAAAAAAAACCCCCCCNCNCNNNGGGGNNNNNAAAAAAAAAAAAAA
AAAAGGGGNNNGGNGNGNAAAAAAAAANNGGGGGGGGGGGGGGNCCCNCANANNCCCCCCCNN
GAAAAAAAAAAAAAAAAACNCCCCNNGGGGGNNAAAAANNANNNTTTTTTTTTTTTTCCCCCCCC
NGGGGGGGGGGGGGGGNCCCCCNNTTTTTTTTTTTTTTNAAANNNGAAAAACCCCCCCCCCNNAA
AAAA

| | |
|---|---|
| Sequence 919 | cMhvSB030c09a2 |

CCCTTTCGAGCGGCCGCCCGGGCAGGTACGCGGGAATGTCATTATGTGACAAACCAATTTTTTTGT
GCCTCTGTTTCCTCATTTGTGAAAATTGGACTAAATAATCTTTAAGGTCTCTTTTTCTTTTGCAGTTC
TAATATCAGTTCCTTGCGCATTTTATATTCATTTTGAAAAGTAATTTATAAGTATTAGTAACTAGAA
GAACCTTTTATTCTAAAATTTTAATATTTAAAAAAAAACACCCCCCCAAAAAACAAGTTCAATGTG
AGGAGCCAGAATCTATCATTTGTAAGTTAAGGCTAAATACAGATTCTGAATTTGAGGTGCTTTAAG
GAAATGAAAAAAAAAAAAAAAAAAAAA

| | |
|---|---|
| Sequence 920 | cMhvSB049g09a2 |

AGGTACGCGGGGGAAAGTGTGTAGCACCTCCACCTTCTCTCTCTCTCTCCCTCTCCCTCTCCTGCCA
GCCAAGTGAAGACATGCTTACTTCCCCTTCACCTTCCTTCATGATGTTACCATTGGAATGACATACT
GCATCCTATAGTTATACCATCCACTCTGAAATCAATGTGAATTTAACTTCAGTTCCATACAGAAAC
TTCTTTTCCACAGATGGAGTTTAAGCCCAAGCTGGAGTGCGATGGTGCAATCCCAACTCACTGCAA
CCTCTGCCTCCCAGGTTCAAGCTATTTTCCTGGCTTAGCCTCCGGAGTAGCTGGAATTACAGATGTG
CGCCCCCATGACCAGTA

| | |
|---|---|
| Sequence 921 | cMhvSB028g01a2 |

GGTACTGAGCTCCACAAACGTGGCCATGGTTGGTGCGGAAATGATTCTGAGTGAGCAGGTAGAAG
TCTCACGTCCTGCTGTGTCCAGAGTTGGTTCCTTCCAGAGGGTTCGTGGTCTCGCTGGCTTCAAGAA
TGAAGCCCGTGGACCTTCACAGTGTGTGTTACAAGCTGTTAAAGATGTTGTGTCTGGAGTTTGTTC
CTTCAGATGTGTCTGGAGTTTCTCCCTTCTGGTGGGTTTGTGGTGTCCCTGACTT

| | |
|---|---|
| Sequence 922 | cMhvSB101e02a2 |

ACTTTTTTTTTTNGTTTTTTTTNGNNANTACNTCCCNGGNTNGGNAGNGGGNAATTNGCCCCCCTGN
TGCNTTCNTTGNATGNGGNACCCGTTTTTAAGGCTCCNTTTCCGNAATNAAACCNTNATTCCCCNT
NACCCNNGGNNACNATGGTAGGNACGGCAACTACNATCAAAAGTTNATAGGGNAAACTTTCAAA
NGGGTCNTCNCCGCCCCCGCTNACNTGCCNGGGCGGCCGCCCGGNNAGGAACTTTTTTTTTTTTTT
TTTTTTTTTTTTAAANAAAAAAAANCCCNTTTTTTTTTTTTTTTNGGGGNNGGGGGNNAAAAAANTT
TTNGGGGGGGGGGGCCNTTTTTTTTTAAA

| | |
|---|---|
| Sequence 923 | cMhvSB105b12a2 |

CCCCGCGGTGGCGGCCGAGGTACAGGTTTGTAGCCAAAAAGCAATAGGCTATACCATAATAGTGC
ANGTGCGTATAAGGCTTTTACATAAAGGTTTTATGACCTGTATGATGTTNACACAACAACAAAATT
GCCTAGTGGTGCATTTACTATAACATATCCCATCCTTAAGGGACACGTGAATGTATATACACACAC
ACACATATACACATATTACCAAATGGATACATACGTGGTTACCTACAGAAAAATTTAAACTTTGAA
ATAATACTCTTAGGGAATGTTACCTTTTTAAAAGATATTCTTTAAATTTATATTTGCTATTATGTGC
CTTACCAATATTCACATGTAACATTGCCATTTCACTAAGGGATTTTTTATATTAGCATTTTAATCAG
CACATTTGGTGGTCTGTTTACCCTGTGTTATGAGTTA

| | |
|---|---|
| Sequence 924 | cMhvSB090b12a2 |

AAATTCNNTGCGCTACTACCACCTGCTGNACATGGAGTCCCTGGCCNCNCANATNCATGGCGTGG
AGTTTTCGNAGTGGCTGCTGAAAAAACTCAAACCGAACNAAGCGCTNTTCCGCCTGGCCGAGGAA
ACGGGCGTCATCCTGTTGCCNGGCCNNNGCTTNAGGACCACNCATCCGTCCGGCCNTTGTCNCTGG
CCAACCTGAACAAATACCACTATGCCAACATCNNGCCGCNCCATCCGCAACATGGCGTCCGANTT
CTTTGCCGTGTTTGAAAAGGAAAANGGCGGC

| | |
|---|---|
| Sequence 925 | cMhvSB091g07a2 |

GACACGCTTTCCTTGAACTGAAATTTCCCCATAAAGAAAAACCANATTTGGAGTTCGTTCTTGAAA
TGTCCTCACCACAACTGATNAAAACACATCTCCCTTCACATNTGATTCCACCATCTATCTGGAAAG
AAAACTGCAAGATTGTCTATGTGGCCAGAAATCCCAAGGATTGCCTGGTGTCCTACTACCACTTTC
ACAGGATGGCTTCCTTTATGCCTGATCCTCAGAACTTANAGGAATTNTATGAGAAATTCATGTCCC
GGAAAAGTTGTTGGCGGGTCCTGGTTTGACCATGTGAAAGGATGGTGGGCTGCAAAAGACATGCA
CCGGATCCTCTACCTCTTCTACGAGGATATTAAAA

| | |
|---|---|
| Sequence 926 | cMhvSB092g04a2 |

AATTGGAGCTCCCCGCGGTGGCGGCCGAGGTACTGAACTCCACAAACNTGGNCATGGTTGGTGCN
GAAATGATTCTGANTGAGCANGTAAAATTNTCACNTCCTGCTGTGTCCAGAGTTGNTTCCTTCCAA
AGGGTTCNTGGTCTCCCTGGCTTCAAAAATNAANCCGGGGACCTTCTCAGNGTGTGTTACAAGCTG
TTAAANATGTTGTGTCTGGAGTTTGTTCCTTCAAATGTGTCTGGAGTTTCTCCCTTCTGGTGGGTTT
GTGGTGTCTNTGACTTCAAGAATTAACCCGGNGACTGTCGTGGNGATCNTTGTAGCTCTTAAAGGG
GGGNGTGNACCCNNACCAGTGGGCATCAGCANGATTTTTCGTCANGAGGGTAAGAACAAAGTTTC
CACNGTGTGGAAGGGTNTCNTGANCGGTTCCCTGCTCCCNTGTACCTNCCCGGGCGGGCGATCTAA
AACTATTGGNTCCCCCGGGCTAANAAGAATTCNATATNAANCTTATCNATTCCGTNGAANCTTNGA
GGGGGGGGCCCNNCAACCCAGGTTTTTGTTT

| | |
|---|---|
| Sequence 927 | cMhvSB017d09a1 |

CCCTTTCGAGCGGCCGCCCGGNCAGGTACAGTCTCTGCTTCACTCCTGGCTACACAATTGAAAGGC
GCATTGGAGGACTGATTTTCCCTCCTTCCTACATACCTATTTGTTATGNTCAAAAATTAAANTTGAT
CAAATGTACTTTTCATGGTANTAGNGGTTAAAATAACANTGAGTCTTATGNTNCNNTTATTTTATT
GAACTTTATTTNGGTTTTTCTCAAANANTGNTGNTGGATTAATTNAAATTANANNTTGTGNNTATT
NCATNGNTTNTTTTTAACCAGNNTGTAANANGTTCTTTTTANGTGGTAAANNTACNTCTCNACCTTT
AANNCTTTTAATTTTATGTATGTAAACCNAAATTGNGNGTGTNAANAANGGCCTTGGAACCCATTT
AATNGGGTCTTTTAATAGTCCNCAAAANAACCTTCNCTTTGGGTNAGGTTANTNTTCNAAANTTTT
NTTCNCTTTCAAATCCCCANTTTTCTTT

| | |
|---|---|
| Sequence 928 | cMhvSB093f01a2 |

ACTTTTTTTTTTTTTTTTTTTTTTTTNAAAAAATNTTTTTTTTTTTTTTTTTTTTTTTTTTTTTTTTTTTTT
TTTTTTTTTTTTTTTTTTTTTTTTTTTTTTTTAAAAAAAAAAAAATTTTNTANATTTTNCCCCCNNNNC
CCCCCCNTTTTTNNGGGGGGGGGGGGGNNAAANAAAAAAAAAAAAANTNTTTTTNNAAAAAAAA
ANCNNNTTTNNGGGNGNACNCAAAAAAAANNNNNGGGNNAAAAAAAAAAANTTTTTTAAAACNT
TTTTTNCNTCAAAAATTTANNCNNCCCNAAAAAAAAAAAAAACNCNTTTTTTTTTTTAAAAA

| | |
|---|---|
| Sequence 929 | cMhvSB029b06a2 |

CCCTTAGCGTGGTCGCGGCCGAGGTACNCGGGGAGGCCATCTCGCTATAGGAAAGGAAAGTGGAA
CAGCATTCATCCTCAACATTTTTACGAAGACAAAATGAAGACTGGAGTAGAAGACTGATCAGTGC
AGGTGTAGCATAAAAGTGTAATCCTGGAAGATGTGGTGTGAGAAGGTANCACAAGTGAANCAGA
NATACANGANATAGGGAAGGGAAGCTGGAANCANAGGTCACTGGAGGGAGAGGGAGATGGGCA
CATTCAGGGCTACAAAGCAAAGTTCTATGTGATTTACTCACCTCTCAATTGTGGGACCCCTCAAAA
TGTGTACANGTACTCTNCCAGTGACATGCTTNTTGACCACAATGGATGAACTGTGCCCAGCATGCC
CACTTTTCAATGCTNCACTTGATCCCCATGTTT

| | |
|---|---|
| Sequence 930 | cMhvSB091d09a2 |

NGAAACTACTACTGAGGGCNAATTGGAGCTCCCCGCGGTGGCGGCCGAGGTACTTTTTTTTTTTTT
TTTTTTTNNTTTTTTTTTTTTTTTTTTTTTTTTTTTTTTTTTTTTTTTTTTTNTNTTTTTTTTTTTTTTTTTT
TTTTTTTTTTTTTTTAAAAAAAAAAAANNNANTTTTTTTTNNAAAAAANANNNNCNNNNGGGGGGG
NNANNNNNNNNNNNTNNTTAAAAAAAAAAAGGGGGNAGNAAAAAAAAAAAAAANNNNTTTTAA
ANNNGGGGCCCCCCCNNNNNNNTTNNATAAANNNAAAAAAAAAAAAAANNTTTTTTNANCCCCC
CCNGGGGGGGGGNTTTTTTNNNCCCCCCCCCCCNCNNTTNTTNTTATTNAAAAANAAGNGGCCCCC
CCCCCCCNAAAAAAAAAANNAATNTNACTNAAANNNTGGGNCCNCATAAAATAAAAAAAANNNN
NNGCCCCCNCTNNGGANAAAAAAAA

| | |
|---|---|
| Sequence 931 | cMhvSB090f03a2 |

GGGCGAATTGGAGCTCCCCGCGGTGGCGGCCGAGGTACGGGAAGGTGAAAAAAAAAAAAAAAAA
AAANCCCCTTTTTTTTTTTTTTTTTTTTTNNTTTTTTTTTTTTTTTTTTTTTTTTTTTTTTTTTTTTTTTTT
TTTTTTTTTTTTTTTTTTTTTTTTTTCTTTNTCTNNNTTTTTTTTTTTTNTTCTTNTTTTTTTTTTTTTAN
GTANCCNCCCNNCCCGCCCNANNTCNTTTNTTTTNTTNCCNCCCCCCCCCCNANNTNNNTTTCNGG
NGGGGNNTNNCTCNCNCNTNTTNNNCANCNCCNCCCCCNGGGGGGGGGG

| | |
|---|---|
| Sequence 932 | cMhvSB105h05a2 |

AGGTACGCGGGGGTTGTGATGTTTTTTTTTTTTTTAAAAAAAAATCCNAANTTTTTAAAAAAAAAA
AAAAAAAAAAAAACCCCCCCCCCCNNNNAAAAAAAAAANNNCCCCCCCCCCCNNNAAANNAANN
TNNAANNTNNTTNAACCCCCCCCCCCCNGGGGGGGNNCCCCCCCCCNNCTTTTTTTNNTTTNANNA
AANAAAAAANACCCCCCCCCCAAAAAAAA

| | |
|---|---|
| Sequence 933 | cMhvSB005h07 |

GATATCTGCAGAATTCGCCCTTAGCGTGGTCGCGGCCCGAGGTACTTTTTTTTTTTTTNTTTTATAN
TNGTTNGGGGTCTTATATGCGCTATGAATATGAATATGACAGCTTCACGGCTCCAACGTAATTATA
GAAAATAAAAATAATATGACATTACTTTGGCAGGCAGGCATACATTTTCATTTAATATGACACAAT
AAGATTACTACTTTCTCCCAAAAGTTAACTCCTATTGCCAATAAAAACTTACTTCTAGTTCTTTAAT
TTTTTCTTCTGCTATTTTC

| | |
|---|---|
| Sequence 934 | cMhvSB008d06 |

CCCTTTCGAGCGGCCCGCCCGGGCAGGTACTGGGATTACAGGTGTGAGCCACCATGCCTGGCCTGT
AAAACTCACTTTCAATACCAGGGATAAGAGGAGGGGCTAAGTGAAGAAGAAATTACTTGAAAAGC
CTAAGAAAACCAGATCTATGCTTACTGCAAAACTTAATTCTGAAAATGTTTTAGTAATTAAATCTG
GCTGTTCAGTTGAGAGAAGAATATGAAACGATGAGGAGTCTCTGAATTTGGAATCTACACAGAAT
GGTGGATTTAGAAGCATAATAGAAATCAGTGCATCTTATTAGCTGCCTTGGTTCTTTGATTGTTTTC
TTCGGGTTCCAAGAATTTTTAGGATCTGAAAATCACGACAAACCAAAACAGAGAGAGATAAATCT
GTGCAGAAAACATCAAATCTATGGCCACCCGCGTACCTCGGCCGCGACCACGCTAAGGG

| | |
|---|---|
| Sequence 935 | cMhvSB012b09 |

CCCTTTCGAGCGGCCGCCCGGGCAGGTACAAGGCATGATGAGTCCTTTTGCTTTTAGGCTTTTGAC
TTCTGGTTTTAGACTTTCTTTAGCTTCTGTTGTTAGACAACATTGTGTAAGCTTGGTTTTTATAAGTT
TGCATGGATTAAACTGAACTTAATGAAATTGTCCCTCCCCCCAAATTCTCAGCACAATTTTTAGGC
CCACAAGGAGTCAAGCACCTCAAGGAGATCTTCAGTTTGAACTTGGTGTAAGACACAGGGATACT
GATGAATCAATATTCAAATTAGCTGTTACCTACTTAAGAAAGAGAGGAGACCTTGGGGATTTCGA
GGAAGGGTTCCGTAAGGGAGATTTTAGCTGAGAAATACCATTTGCACAGTCAATCACTTCTGACCA
AAGTTATCAGAAAAAGGAGAAAAAG

| | |
|---|---|
| Sequence 936 | cMhvSB016a08 |

CCCTTTCGAGCGGCGCCCGGGCAGGTACGCGGGGGCCATAGTGAAGAAGGAACTGCTGTCTGTGG
TGGCTGGGGGAGACAACTACAGGGTCAATAACAAGCACGATGACAGATACACACCACTGCCTTCC
AACAAAATCGTCAAGCGGGCAGAGGAGTTGGTGGGGCAGGAGTTGCCTTATTCGCTGACCAGTGA
CAACTGCGAGCACTTCGTGAACCATCTGCGCTATGGCGTCTCCCGCAGTGACCAGGTCACTGGTGC
AGTCACGACAGTAGGTGTGGCAGCAGGCCTGCTGGCTGCCGCAAGCCTTGTGGGGGATCCTGCTT
GGCCAGAAAGCAAGCGGGAAAGGCAATAAATCCAAGAAATTGTNCCAACAACCACCAATTCTTAC
NGAGGAATATTATTTAACCAGCAAGGAGTGGAGGTTTGGTTTACTGATTTTACTGNTTTGGGNTCA
TGAAATTTTATTTTAATGGGAGTTAAAAACACAGGAAAATGTATTTNGAAATGCAACTTAATATTG
AATTTTTTAAAAGACACAATTNGGCTTTTGGAAA

| | |
|---|---|
| Sequence 937 | cMhvSB018h05 |

CCCTTTCGAGCGGCCGCCCGGGCAGGTACTGGATCAGTTTCTCCTGCGTGAGGTATGGGTGACACT
CAACCTGCANCANCAAACAATCCTCATCACGGGGAAAGCCGGCTCTGTTTTGCATTGTTCCTAGGG
AGTTCTGGTTAAGTCACTGGTTTATATTTCAAGTCCAGGTTTGTTCAAGAGCCTCTCGATCTGGAAG
TGGTTGAAATTTGAGACCCCAAGGGCTTTCACCAGCCCCTCGTCCACCAGCTCCTCCATGGCCTCC
AGGCATCCAAGAACGTTCCTTTTCCACTGATCATATTACCTTTATCATCTTTGGGGAAAAAGTCATC
CCCAAGTCTTGAATCCCTGTGGCCAAGTGAAATAAGATAGACGTTCAGATAGCCCAGCTTCANGTC
CTTGAGGGTCTTCTTCAAANGCTTTCCTCACAAAGGGGTCTCTCAAAAGAAAGTGGGCCACACCTT
GCTGACGATGAACAAGGTCCTNCCGCATNAAAACCCTTCTTTGGGATCCTTTTCTTGGATGGCTTCT
TCCCACCTCATGTTGAATTCTNATAAAAATAGGGCNCAGTCNAATGTNGCGATATTCTTGCATTNA
ATNGGCCACCTTTACCGGTTTTTTTTA

| | |
|---|---|
| Sequence 938 | cMhvSB020g05 |

CCCTTAGCGTGGTCGCGGCCGAGGTACGCGGGGAGGGAACCGCTCAGATACCCTTCCACACCGTG
GAAACTTTGTTCTTACCCTNTTGACAAAAAATCTTGCTGCTGCTCACTCTTTGGGTCCACACCACCT
TTAAGAGCTACAACGATCACCACGACAGTCTGCGGCTTCATTCTTGAAGTCAGCGACACCACAAAC
CCACCAGAAGGGAGAAACTCCANACACATCTGAAGGAACAAACTCCAGACACAACATCTTTAACA
GCTGTAACACACACTGTGAAGGTCCACGGCTTCATTCTTGAAGCCAGCGAGACCACGAACCCTTTG
GAAGGAACCAACTCTGGACACAAGCAAGACCGTGAGACTTCTACCTGCTCACTCAAAATCATTTCC
G

| | |
|---|---|
| Sequence 939 | cMhvSB023a03 |

CCCTTAGCGTGGTCGCGGCCGAGGTCGGCCGAGGTACAAAAGCCAAGATGCCCATTGTGGGCCTG
GGCACTTGGAGGTCTCTTCTCGGCAAAGTGAAAGAAGCGGTGAAGGTGGCCATTGATGCAGAATA
TCGCCACATTGACTGTGCCTATTTCTATGAGAATCAACATGAGGTGGGAGAAGCCATCCAAGNAG
AAGATCCAAGAGAAGGCTGTGATGCGGGAGGACCTGTTCATCGTCAGCAAGGTTGTGGCCCCACT
TTCTTTGAGAGACCCCCTTTGTGAGGAAAGCCCTTTTGAGAAAGACCCTTCAAGGGACTTGNAANC
TGNNCCTATCTGGAACCNTTCTATCTTTATTCACTTGGCCACAAGGGGGATTTCAAGNACTGGGGG
GGATNGGACTTTTTT

| | |
|---|---|
| Sequence 940 | cMhvSB023a03 |

CCCTTAGCGTGGTCGCGGCCGAGGTCGGCCGAGGTACAAAAGCCAAGATGCCCATTGTGGGCCTG
GGCACTTGGAGGTCTCTTCTCGGCAAAGTGAAAGAAGCGGTGAAGGTGGCCATTGATGCAGAATA
TCGCCACATTGACTGTGCCTATTTCTATGAGAATCAACATGAGGTGGGAGAAGCCATCCAAGNAG
AAGATCCAAGAGAAGGCTGTGATGCGGGAGGACCTGTTCATCGTCAGCAAGGTTGTGGCCCCACT
TTCTTTGAGAGACCCCCTTTGTGAGGAAAGCCCTTTTGAGAAAGACCCTTCAAGGGACTTGNAANC
TGNNCCTATCTGGAACCNTTCTATCTTTATTCACTTGGCCACAAGGGGGATTTCAAGNACTGGGGG
GGATNGGACTTTTTT

| | |
|---|---|
| Sequence 941 | cMhvSB027e12 |

CCCTTAGCGTGGTCGCGGCCGAGGTACCCTGCGCTGGCTCCGTGAACCTTAGGGACAACACCGGG
ACACCCGCGAGGCCGGAAAATGGACTCAGTGGCTTTTGAGGATGTGTCTGTGAGCTTCAGCCAGG
AGGAGTGGGCTCTGCTGGCTCCTTCACAGAAGAAACTCTACAGAGATGTGATGCAGGAAACATTC
AAGAACCTGGCATCTATAGGGGAAAAATGGGAAGACCCGAATGTTGAAGATCAACACAAAAACC
AAGGACGAAATCTAAGAAGCCATACGGGAGAGAGACTCTGTGAAGGTAAAGAAGGTAGTCAATG
TGCAGAAAACTTCAGTCCCAATCTCAGTGTGACGAAGAAGACTGCCGGAGTAAAACCATATGAGT
GTACCTGCCCGGGCGGCCGCTCGAAAGGGCGAA

| | |
|---|---|
| Sequence 942 | cMhvSB028b02 |

CCCTTTCGAGCGGCCGCCCGGGCAGGTACANNNGNNCAGATNCCNNTTNTGGGCCNGNGCACTNT
ANNGTCTNTTCTTGGNAAANTNNAAGNCTCCCNTANNGACNCCATTNNNCCGGAATATCACCNCA
TTGACTCGGCCTATATCTNTGAGAANCANCTTCNACATGGCAAAANCCCTCCAAGACACACATNCT
TACACNACTCTCNACATNCCGGAAGGNACCTGCTAATCGTCANCAAGGTGTGGCCCACTTTCTTTG
AGAGACCCCTNNTGANGAANGCCTTTGAGAAACCCTCGGGACCTGAAGCTGAGCTATCTGGACGT
CTATCTTATTCACTGGCCACAGGGATTCAAGACTGGGGATGACTTTTTCCCCAAAGATGATAAAGG
TAATATGATCAGTGGAAAAGGAACCTTCTTGG

| | |
|---|---|
| Sequence 943 | cMhvSB034g12 |

CCCTTTCGAGCGGCCGCCCGGGCAGGTACTGGTGAACTCCCTCACTTGAATTTCTCGTTCTTATGAA
GGTGCTTTCTTGCTTGGATAGTTGTTCAGTGTGACATTCCTGCAGGGTGAACAATTGCTAGAGGGT
TCTATTCAGCCATCTTTCTCCACCTCACATCCATGTTTTTGCATGTTATTTCTTTCCTTTTATTGATTA
GCATTTGATTCCATGAATATAGCACAATGTATATAACCACTATTCTTTTCTGGAAAACTTATGTCCA
GGTTGGGGTTATTATGAATAAGGCTATGAAATTTCAGGTACCTCGGCCGCGACCACGCTAAGGG

| | |
|---|---|
| Sequence 944 | cMhvSB038d05 |

CCCTTAGCGTGGTCGCGGCCGAGGTACAAATCTGTTGCCAGCCTGAACACACCTGTAGGAGGTGG
ATGGAGACCCTGGTTGAGAGGTCTCACCCAGCCAGTAGAAACAGGATCAGGGACCTGCTTGAAGA
AGCAGTCTAGCCCCACTTTTGTAGAACAACTGAGCTGTGCTGGGATACCATTTCTGCCCCTCATGG
TGTTGGGTTCTCCAAAACCTGGAAGCTGGAACGGCTAAATTGCAGAAACAGCAAAGATGGCAGCC
TGCCCCTCTCTCTAGTAACTCTGTCCCAGGATGCTTTCAAACCCTTGTCAACCAGAGAACATCAGT
GGGAGAGGCTGAAGACCCTGGTTGGGAAGTTCTCCCAAGTGAGGAGGAACAGATCAGGGACCTGC
TTAAAGAAGCGGTCTTGCCACGCTTTTGTAGAGCAGTTGTGTCATGCTGGGGTACCTGCCCGGGC

| | |
|---|---|
| Sequence 945 | cMhvSB041a06 |

ACAAAAGCCAAGATGCCCATTGTGGGCCTGGGCACTTGGAGGTCTCTTCTCGNCAAAGTTGANNG
AAGCGGTTGAAGGTGGCCATTGATGCAGAATATCGGCCACATTGACTGTGCCTATTTCTATGAGAA
TCAACATGAGGTGGGAGAAGCCATCCAAGAGAAGATCCAAGAGAAGGCTGTGATGCGGGAGGAC
CTGTTCATCGTCAGNAAGGTGTGGCCCACTTTCTTTGAGAGACCCCTTGTGGAGGNAAAGCCTTTG
AGAAGACCCTCAAGGGACCNTGGAAGCTTGAAGCCTATCTGGGACCGTCTATTCTTAATTCACTTG
GCCCACCAGGGGATTTCAAGGACCTGGGGGGATTGACCTTTTTCCCCAAAGGATGGATTAAAAGG
GTAAATTATGGATCAGGTGGNAAAAAGGGAACCGTTNCTTGGGATGCCTGGGNAGGNCCATGGGA
GGGAGCTTGGTGGGGACCGAAGGGGGCTTGGGTGNAAAGCCCCTTGGGGGTCTTCAAAATTTCAA
CCCCACTTCCAGNATCCGAAGAGGCTCTTTGAACCAAAACCTGGACTGGAAATATAANACCCAGT
GGACTAACCCAGGTTTGAGTGGTCACCCATTCCTTAACGCCAGGAAGAAAACCTTGATCCAAGTTN
CCCTTCCGGGCCCGCCTCNTANNAACTTAGGTGGGAATNCCCCCCGGGGGCTTGCCANGGAAATTC
CNNATTATCCAAAGCCTTTATCCGGATTNCCCGGCCCGAACCTTCCGGANGGGGGGGGG

| | |
|---|---|
| Sequence 946 | cMhvSB042c03 |

TCCACCGCGGTGGCGGCCGAGGTACAGTGGGAGAGTGAGGTGGGAGAAGAAGAGTGTCTGGTAG
GTGTGCTCACTGTCTTCTTGGCTGAGAATGTTNAATTGGAAGAGTGGGCCGCTCAGAGCTCCTACA
AAGGCAGAGCAAAGCTTCTTAGCTGACATTGTTTGAGAAATTGTTGGCAGGCTCTGGAATGCTTGT
TTGGCTTTCTTGCGGTGCCTTTGGTGTCTTGTTTTTCTTCACATTGCCCTTGAAATGATCACAGGGG
GCACTGCTTCTTTGGCAGCCCANACACTGTCATGAATTTTTCTTCTCGGGGCTCCTCAANGAACCA
AATCTTTTGCACCTCACATTTCTTGGGCCCGCCTTTNCTGGGGAAGCCATCCTCCTTAGAAGCCTGG
CCCTCGGTCCCCTTGTGGGGNCTNTTGGCCGACCCCCCTTGGGAATNTTCAGGGCTGCTTAGAAGA
ACCCATTGGGACCATTCAAGCCATTTAAGTTGGGCAAGNCAAACCAGGGGAAGGGAAGGGGGAA
ANNANNATTTTNAGAAAACCTTTTTCA

| | |
|---|---|
| Sequence 947 | cMhvSB042e02 |

TGGGCCGGGAGGCAGTGCTGATCCGGCTGCTCCTCCAGCCCTTCAGACGAGATCCTGTTTCAGCTA
AATGCAGGGAAACTCAATGTTTTTTTAAGTTTTGTTTTCCCTTTAAAGCCTTTTTTTAGGCCACATT
GACAGTGGTGGGCGGGGAGAAGATAGGGAACACTCATCCCTGGTCGTCTATCCCAGTGTGTGTTTT
AACATTTCACAGCCCANGAACCCACAGATGTGTCTGGGAGAGCCTGGCAAGGCATTCCTCATCAC
CATCGTTGTTTGCAAAAGGTTTAAAACAAAAACAAAAAAAACCACNTCTGNAAAAANANATNNGN
TTATATTATAGAATNNNAGTTTCCCTTTNGGGNCCCGGCTTCTTANGAAACCTANGGTGGNNATTC
CCCCCCCGGGGGCCTGGCCAAGGGGAAATTTCCGAATTNTTCCAAAGGCCTTTTATTTCGGAATTN
NCCCCGGTNCGNACCCCTTCNGNNAGGGGGGGGGGGGGGCCCCCCGGGGTANCCCCCAAGCCTTT
TTTTGGTTTCCCCCNTTTTAAAGGTGGGNGGGGGG

| | |
|---|---|
| Sequence 948 | cMhvSB042e11 |

NCCTGNCAGGTACTGTNCTCNACAAACGNGGGNATNNTNGGAGCTNAATTGNGTTAAGACATCAG
GCTCCANATATGAACTTTCAGCANAAGCGCTTGCCGGGAGCAAAGGGACAGAAAAGCTGANATGA
ACAGTGCCTGGCAACAATCACAGCCGGGCAAGGGNGCTCCGAGCCTCGCATCCCC

| | |
|---|---|
| Sequence 949 | cMhvSB042e11 |

TCGAGGGGGGGGCCCCGGGTANCCCANNNTTTTTGTATCCCTTTTTANGNGGAGGGGTTAAATTTG
CGCCGCTTGGCCGTTAATCAATGGTCATTANCTGGTTTTCCTTGGTGTGGAAAATTGTTTATTCCCG
CTCACAAATTCCACCACCAAANATTACGAAGCCCGGGGAAGCATAAAAAGNTGGTAAAAAGCCCT
GGGGG

| | |
|---|---|
| Sequence 950 | cMhvSB044c01 |

CTNCCTGAGGGCGAATTGGAGCTCCCCGCGGTGGCGGCCGAGGTACTGATTGGGGAAGTGATAAA
TGTTCATGAAATCTTCACAATTTATGTTCAGAGATTGCAGTAAAGACAGGCGTAAGAAATTATAAA
AATATTAATGTGGGGAATTAAGAAATGTCCATGAAATCTTCACAATTTATGTTCTTCTGCCATGGC
TTCAGCCAGTCTCTCTGTTGGGGGTCCCTGAATTCCTGCAACAGCTCAGAAACTAGAGGCTGAGAA
AGGGAGTCACTCAAACCTTGAATCCCTGTGGCCAGTGAATAAGATAGACGTCCAGATAGCTCAGC
TTCAGGTCCTTGAGGGTCTTCTCAAAGGCTTTCCTCACAAGGGGTCTCTCAAAGAAAGT

| | |
|---|---|
| Sequence 951 | cMhvSB044c06 |

CCGGGCAGGTACGCGGGGACAGCTGGGAGGACACCCACATGGTCGGCGTGCAGGATATTTCGCTG
GACCCTAGAAAAGCCACCACGACCTGTGGGCCATGATGCTACCCCAATGGCTGCTGCTGCTGTTCC
TTCTCTTCTCCTTTCTCTTCCTCCTCACCAGGGGCTCACTTTCTCCAACAAAATACAACCTTTTGTCC
CCTCCAGGCATCCACCGTCTGCACAGACTTACCCCGGCCCCCACGTAGAGACCACCCTGCCTGGCA
GGAGCCAGAGGAGCTCAAGGAGTCTTGCATCCGGAACCAGGACTGCGAGACTGGCTGCTGCCAAC
GTGCTC

| | |
|---|---|
| Sequence 952 | cMhvSB045d05 |

TTTGAGAAGCCAGCGCTCACCCACCCGGGGTCTCTGTGCATTGACCTTTGGGTGCTGACTTGGAGA
AAAGCACAAACACGACCAGTCCCATCCTGGCTCCCGTGGGGCTTCTTCTATCTACGCATTGTATCG
ACTGCATTAGTTGGACTAAGATGATGACTCAGTTAAAGGAGGAGACAAATGCTGACTGTCTAAGC
AAGAATGGCCCAAGCTGGCAAGAAAAAGCACACTGCATACATAGGATACAGAAGGGGCAGGAGC
TTCTGCCTGCCGGGATCTGCAACCATTTACATTTTGTTTTGCCTGCAAAACCTATNAAGNAAGGGA
TTTCCTGTTTGGCCCAGGGGAGTCTTCCACTGGAACAAACAAAAATGGGCAGTTCAAAAAGGTTCT
TGGAGGTGGTCCCTTATTCCAAGCCAGCCCAGGAGTCCCTTCATCCGTCATNCCACGGGGAAGAGT
CTTTTGAGGGGGAAACATGGAAGTCCANGCTCATGCCTCTGCCTATGGGGTNCAATTTCTTTCGGG
GAATCACNTGTGGATCATGGATATNTTTCATTAACCCCCTTGCGGGACCCACCNATGGTTTTCAAG
GGGTGGCTTTTTNCCCCCTTTTT

| | |
|---|---|
| Sequence 953 | cMhvSB045d08 |

TTGTCAGCTGTGAGCGTTGCGGGGCTGGTGGGGTGTGTTTGAGTATGTAAGTGTCTATTTCCTGTGC
TCTAACAGTGACTATTTCAGTTCTAACCCTTCAATTGCTAATTGGATGGGGGAATGGCCTCTTAGAT
TGTCCTTGTTTTGACTTATCTGCTAAGGCGAGAGAATGTCTGGGTTTGCCACACAGTCCCGCAGGG
ACCCCTGCTCTTTGCCAGGATTTTTATATCAAGTACCT

| | |
|---|---|
| Sequence 954 | cMhvSB045d08 |

ATTCCGATATCAAGCTTATCGGATACTCGTACGACCCTCGGAGGNGNGGGGGCCCGGGATACCCC
AGCNTTTTTGTTTCCNTTTTAANTGGAGGGGTTTAAATTGCCGCCGCCTTGGNCGTTAAATTCATGG
GTTCATAGCCTGTTTCCTGTGTGGAAAATTGTTAATCCCGGCTCACAAATTNCACACNAAACNATA
ANGAAGCCNCGGGGGAGGCAATAAAAGGTGGTAAAAAGANCCTGGCGNNTGCCCCTAAATNGAA
NTTNNAANCTAAAGNTTNAANCATTGTCAAATTTGNCNGTTTGGCCGCCTTCAACTTGGNCCCCGC
TTTTTTNCANGTCNGGGGGGAAA

| | |
|---|---|
| Sequence 955 | cMhvSB045f05 |

ATGGGCGAATTGGACTCCACCGCGGTGGCGGCCGTCGCCATGGTGAANCTGAGCAAAGAGGCCAA
GCAGAGACTACAGCAGCTCTTCAAGGGGAGCCAGTTTGCCATTCGCTGGGGCTTTATCCCTCTTGT
GATTTACCTGGGATTTAAGAGGGGTGCAGATCCCGGAATGCCTGAACCAACTGTTTTGAGCCTACT
TTGGGGATAAAGGATTATTTGGTCTTCTGGATTTGGAGGCAATCAGCGGACAGCATGGAAGATGT
GTGCTCTGGCTCGGATAAGAGATGGGNCATCATTCAGTCACCTAGTTGGGATGGCACCAAGGCTCT
TCACAGNACGCATNTGTTAGCNAGCAGTGGGCAACTTGGTACCTCGGCCCGCTCTANTAACCTAGG
TGGGATCCCCCGGGCCTGCAAGGNAATTCGATATCAAGCCTTTATCCGATACCCGTGCGACCTCNA
GGGGGGGGGGCCCGGTACCCCAGCTTTTTGTTCCCCTTTAGTGAGGGGTTTAAATTGGCGCCGCTT
GGCGTAATCATGGGTCAATAAGCTGGAATCCTGTGTGGAAATTGNTTATTCCCGCTCA

| | |
|---|---|
| Sequence 956 | cMhvSB046a03 |

AGGTACAAAAGCCAAGATGCCCATTGTGGGCCTGGGCACTTGGAGGNCTCTTCTCGGCAAAGTGA
AAGAAGCGGTGAAGGTGGCCATTGATGCAGAATATCGCCACATTGACTGTGCCTATTTCTATGAGA
ATCAACATGAGGTGGGAGAAGCCATCCAAGAGAAGATCCAAGAGAAGGCTGTGATGCGGGAGGA
CCTGTTCATCGTCAGCAAGGTGTGGCCCACTTTCTTTGAGAGACCCCTTGTGAGGAAAGCCTTTGA
GAAGACCCTCAAGGGACCTGAAAGCTGAGCCTATCCTGGGACGTCTATCTTATTTCACTTGGCCAC
AGGGGATTCAAGGACTGGGGGATGGACTTTTTCCCCAAAAGATGATAAAAGGTNAAATATNGATC
CAGTGGGAAAAAGGGAACCGTTCTTGGGATGCCCTGGGGGAGGCCCATGGGGAGGGAGCTGGTG
GGACCGAAGGGGGCCTGGTTGAAAAGCCCCTTTGGGGTTCTTCAAAATTTTCAACCCACTTTTCCA
GGAATCCGAAGAGGGGCTCTTTTGAAACAAAAACCCTTGGGACTTGGAAAATTATTAAAAACCCA
AGTGGACCTTNAACCCCAGGGNTTTGGAAGTTGGTTCCACCCCCCATTACCCTTTCACCGGCCAAG
GGAAGGAAAAACCTTGGATTCCCCAGGTTACCCTTTGGCCCCCGGGGGGGCCGGGGCCGCCTTCTT
AAAAAACTNAGATGGGAATCCCCCCCCGGGGCCTTGCCAGNGGAAATTNCGGATNATNAAAGNCT
TTNTCTGAATTACCCNGNCGGAANCNTTNGNNGGGGGGGGGGGC

| | |
|---|---|
| Sequence 957 | cMhvSB046c07 |

GGCGGCCGAGGTACAAAGTGTGAGGTAGGCCACCCAGAAACACCAACTCCGAAGAAATGGAGTC
AGTTTTCCGAAGTAGGGAGTGAAGGCTTCATTTATGTGGGCTGAGACAGTGGAGTTTTTAGCAGGA
TTACAACATTATTCATACAAGGTTGGTGTGTATGTTATAGCAATTTGATTGGCTCTAGGTGATGTTT
CTTTTTGGGGAGGGGATATTTAACATTTTCTTAACAGAGGGTGTAATAAGTCCTGGGTTTTCTTTCA
CCTGGTCTAAGCGAAGCAGGGCAATGAAGGGGGAGTTAATCTACAACAAGGGTCATTAATTCAGA
GGGCGGGAGGCTTTTGACCCTGACATGGTTTCCCTTTAGTCAATGTACCTGCCCGGGCGGC

| | |
|---|---|
| Sequence 958 | cMhvSB047f10 |

AGGTACGCGGGAGCAGGGAACTGCTCAGATACCCTTCCACACCGTGGAAACTTTGTTCTTACCCTC
TTGACGAAAAATCTTGCTGCTGCTCACTCTTTGGGTCCACACCACCTTTAAGAGCTACAACGATCA
TCACGACAGTCTGCGGCTTCATTCTTGAAGTCAGCGACACCCCAAACCCACCAGAAGGGAGAAAC
TCCAGGCACATCTGAAGGAACAAACTCCAGACACAACATCTTTAACAGCTGTAACACACACTGTG
AAGGTCCACGGCTTCATTCTTGAAGCCAGCGAGACCACGAACCCTCTGGAAGGAACCAACTCTGG
ACACAGCAGGACGTGAGACTTCTACCTGCTCACTCAGAATCATTTCCGCACCAACCATGGCCACGT
TTGTGGAGCTCAGTACAAAAGCCAAGATGCCCATTGTGGGCCTGGG

| | |
|---|---|
| Sequence 959 | cMhvSB048g07 |

AGGTACAAAAGCCAAGATGCCCATTGTGGGCCTGGGCACTTGGAGGTCTCTTCTCNNNAAANNNA
AAGTTTANCGNNCGCCCGGGCAGGTACTGGATCAGTTTCTCCTGCGTGAGGTATGGNTGACACTCA
ACCTGGNTAGTCACTGGTTTATATTTCANTCCAGGTTTGNTCAANAGCCTCTCGATCTGGAAGTGG
TTGAAATTTGANACCCCAANGGCTTTCACCAGCCCCTCGTCCACCANCTCCTCCATGGCCTCCCAG
GCATCCAAGAACGTTCCTTTTCCACTTGATCATATTACCTT

| | |
|---|---|
| Sequence 960 | cMhvSB051a06 |

GGCCCGNCCGGGCAGGTACANANGCCAAGATGCCCATTGTGGGCCTGGGCACTTGGAGGTCTCTT
CTCGGCAAAGTGAAAGAAGCGGTGAAGGTGGCCATTGATGCANAATATCGCCACATTGACTGTGC
CTATTTCTATGAGAATCAACATGAGGTGGGAGAAGCCATCCAAGAGAAGATCCAAGAGAAGGCTG
TGATGCGGGAGGACCTGTTCATCNTCAGCAAGGTGTGGCCCACTTTNTTTGAGAGACCCCTTGTGA
GGAAAGCCTTTGAAGAAGACCCTCAAGGACCTGAAAGCTGAAGCTATCTGGGACGTCTTATTCTTT
ATTCACTGGCCCACAGGGATTCAAAGACTGGGGGGATGACTTTTTCCCCAAAAGATGATAAAAGG
GTNATTATTGGATTCAGTGGGAAAAAAGGGAACCGTTTCTTGGGATTGCCCTGGGGAGGCCCATG
GAAGGAGCCTGGTGGGACNAAGGGGCTTGGTTGGAAAAGCCCCTTTGGGGGTNCTCAAAATTTTN
AACCCACTTTCCAGAATCCGAAGANGGCTTCTTNGAAACAAAACCTTGGGANCTGGAAAATATAA
AACCCAGTGGACTTAACCCAGGGTTGGAGTTGTTACCCCATTACCCTTTACGCCAGGGAANAAAAC
TGGATNCCAAGTTACCCTTCNGGNCCGCTTCTTNANAACTTTGTNGGGATTNCCCCCCGGGGCCTG
GGAGGGGAAATTTCGATTNTTNAAAGGCCTTATTCGNTANCCCCGTCGGACCCCTCCTANGGGGG
GGGGG

| | |
|---|---|
| Sequence 961 | cMhvSB054d05 |

NANCTCCACCGCGGTGGCTGACGGATGAGGACTCTGGGCTGCTGGAATAGGACACTCAAGACTTT
TGGCTGCCATTTTGTTTGTTCAGTGGAGACTCCCTGGCCAACAGAATCCTTCTTGATAGTTTGCAGG
CAAAACAAATGTAATGTTGCAGATCCGCAGGCAGAAGCTCTGCCCTTCTGTATCCTATGTATGCAG
NGTGCTTTTTCTTGCCAGCTTGGGCCATTCTTGCTTAGACAGTCAGCATTTGTCTCCTCCTTTAACTG
AGTCATCATCTTAAGTCCAACTAATGCAGTCGATACAAATGCCGTAGATAGGAAGGAAGCCCCAC
GGGGGAGCCAGGGATGGGACTTGGTCCGTGTTTGTGCTTTTCTCCAAGTCAGCACCCAAAGGTCAA
TGCACAGAAGACCCCCGGGTGGGGTNGAAGCCGCTGGCTTCTTCAAAACCGGCNCGCTCTTAGGA
ACTAAGTNGGGATCCCCCGGGGGCTTGGCAGGGAATTCGATAATCAAAGNCTTATCCGATNCCCG
TNCGACCCTNGGAGGGGGGGGCCCGGGNACCCCANCTTTTTGGGTCCCTTTAAGTG

| | |
|---|---|
| Sequence 962 | cMhvSB057c03 |

CCGGGCAGGTACGCGGGGAGCAGGGAACTCGCTCAGATACCCTTCCACACCGTGGAAACTTTGTT
CTTACCCTCTTGACGAAAAATCTTGCTGCTGCTCACTCTTTGGGTCCACACCACCTTTAAGAGCTAC
AACGATCACCACGACAGTCTGCGGCTTCATTCTTGAAGTCAGCGACACCACAAACCCACCAGAAG
GGAGAAACTCCANACACATCTGAAGGAACAAACTCCAGACACAACATCTTTAACAGCTGTAACAC
ACACTTGTGAAGGGTTCCACCGGCTTTCATTTCTTGGAAGCCAGNCGGAGACCCACCGAACCCTTN
TGGGAAAGGGAACCAACTTCTTGGGACACAGGCANGGGACGTTGAANACTTTCTACCTGCTNACT
TCAGAAATNAATTTTCCGGCACCCAACCCCATTGGGCCACGTTTTNGTGNGGAGCTTCAGTACCAA
AAAGCCAAGGATTGCCCCATTTGTTGGGCCCTGGGCCACTTTGGGAGGGTCTCCTTCTTTCGGGNA
AAAANATGGAAAAANAAANCCGGGTGGAAAAGGTG

| | |
|---|---|
| Sequence 963 | cMhvSB060b04 |

AGGTACTTTCTACACAGAACCAAGTAAAGAGAAGGAGGCCGGAACTACACCAGCAAAAGACTGG
ACCCTTGTCGAAACTCCTCCTGGGGAGGAACAAGCCAAGCAGAATGCCAACTCCCAGCTGTCCAT
CTTGTTCATTGAAAAACCTCAAGGAGGAACAGTGAAAGTTGGTGAAGATATCACCTTCATAGCCA
AAGTCAAGGCTGAAGATCTTNTGAGAAAACCCACTATCAAATGGTTCAAAGGAAAATGGATGGAC
CTGGCCAGCAAAGCCGGGAAGCACCTTCAGCTGAAAGGAAACCTTTTGAGAGGCACAGTCGGGTG
TTACCTTGCCCGGGCGGC

| | |
|---|---|
| Sequence 964 | cMhvSB060b04 |

GCTGCAGGAATTTCGGATATTCAAAGCTTTATCGATTACCCGGTCCGACCTCGAAGGGGGGGGCCC
CGGTACCCCANCTTTTGTTCC

| | |
|---|---|
| Sequence 965 | cMhvSB075a08 |

AATTGGAGCTCCCCGCGGTGGCGGCCGATGTACAANTACCGGAATGCCCNTTNTGGGCNAGNNCA
CTNNNAGGCNTATNNTTNCCGAAGANCTNGANGNGGGGNCCGTGGCCCTTGATGCAGAANCTTTA
CNCATTGGCTGTNCCTCTNCTTGTCNTAATCATNGTNATGTGNGANAACNNATCCAAGAGAAGATC
CAAGAGAAGGCTGTGATGCGGGAGGACCTGTTCATCGTCAGCAAGGTGTGGCCCACTTTCTTTGAG
AGACCCCTTGTGAGGAAAGCCTTTGAGAAGACCCTCAAGGACCTGAAGCTGAGCTATCTGGACGT
CTATCTTATTCACTGGCCCAGGGATTCAAGACTGGGGATGACTTTTTCCCCAAAGATGATAAAGGT
AATATGATCAGTGGAAAAGGAACGTTCTTG

| | |
|---|---|
| Sequence 966 | cMhvSB075a10 |

AGGGCGAATTGGAGCTCCCCGCGGTGGCGGCCCGANGTACTGATCTCCACAAACGTGGCCNTGGT
NGGTGCGGAAATGATNNTNAGTGANCNGGTAAAANTCTCACGTNCTGCTGTGNCCAGAGTTGGTT
CCTTNCAGAGGGNTCGNGGTCTCCCTNGCTTCAANAATNAAGCCTTGGACCTTCACAGTGTGTGTT
ACAGCTGTTAAAGATGTTGTGTCTGGAGTTTGTTCCTTCAGATGTGTCTGGAGTTTCTCCCTTCT

| | |
|---|---|
| Sequence 967 | cMhvSB082f05 |

CATNACATNCNNCTATTGGATCTTCTNTNGNATGGNNNTTCCNACNTAATGTTNATTNTNNTAGAA
ATNNGCACNGGNNNNGNGGCNANNTTCTGCATCAATGNCCACCTANGCCGATTNTTTCACTTNGC
CNANAANAGACCTTNAANTGCCCATGCCCACAATGGGCATCTTGGCTTTTGTACCT

| | |
|---|---|
| Sequence 968 | cMhvSB083a12 |

AAGCTCCACAAACGTGGTNATGGTTGGTGCGGAAATGATTCTGAGTGAGCAGGTAGAAGTCTCAC
GTCCTGCTGTGTCCAGAGTTGGTTCCTTCCAGAGGGTTCGTGGTCTCGCTGGCTTCA

| | |
|---|---|
| Sequence 969 | cMhvSB083a12 |

AAGCTCCACAAACGTGGTNATGGTTGGTGCGGAAATGATTCTGAGTGAGCAGGTAGAAGTCTCAC
GTCCTGCTGTGTCCAGAGTTGGTTCCTTCCAGAGGGTTCGTGGTCTCGCTGGCTTCA

| | |
|---|---|
| Sequence 970 | cMhvSB086c06 |

CTCCCCGCGGNGGCGGCCNTCCGGGCAGGTNTTAAAGCCATTTTGCCCANNGTGGGCCTGGGCAC
TGGGNGGTTTCNAANCNNCAAAGTGAAAGAAGCGGTGAAGGTGGCCATTGATGCAGAATATCGCC
ACATTGACTGTGCCTATTTCTATGAGAATCAACATGAGGTGGGAGAAAGC

| | |
|---|---|
| Sequence 971 | cMhvSB088e07 |

AGCTCCACCGCGGTGGTCGAGCGGCCGCCCGGGCAGGTACGCGGGGCTCTCTCGCCAGGCGTCCT
CGTGGAAGTGACATCGTCTTTAAACCCTGCGTGGCAATCCCTGACGCACCGCCGTGATGCCCAGGG
AAGACAGGGCGACCTGGAAGTCCAACTACTTCCTTAAGATCATCCAACTATTGGATGATTATCCGA
AATGTTTCATTGTGGGAGCAGACAATGTGGGCTCCAAGCAGATGCAGCAGATCCGCATGTCCCTTC
NCGGGAAGGCTGTGGTGTTGATGGGCAAAGAACACCATGATGCGCAAGGCCATCCCGAGGGCACC
TGGAAAACAACCCANCTCTGGAGAAACTGCTGCCTCATATCCGGGGGAATGTGGGCTTTGTGTTCA
CCAAGGAGGACCTCACTGAGATCAGGGACATGTTGCTNGCCAATAAGGTGCCACTGCTGCCCGTG
CTGGTGCCATTGNCCCATGTNAAGTNACTGTNNCAGCNCAANAAACACTTNTNTTTNGGCCCTAGA
AAGAACTTCTTTTTTTCNAGGCTTTTANGTTATTNACCACTTAAAATTNTTTNAAGGNGGCACCATT
TTGAAANTCCTTNAGTNGATTNTNNCACCTTNATNAAANAACTTGNNANAACAAAANTNNGGGAN
CCCAANTNAAACCCACCCCTTNTTTNNAAACATTNCTTTAAAAANTTTNCCCCCTTTTTTC

| | |
|---|---|
| Sequence 972 | cMhvSB092f06 |

ACACAGCCTTCAACCCATTTCCTGGCATACAACTCCTAACATCCCGAGAATATCCAAAGTGATGCC
CTTTTCTAATGTTGACTGATGGATGGAAGCCCATAGTTAGCTTCAGAATTAGGGCTGCTCACCAGA
AAGACCAAGGCATGATTACAGAATTAGAACTTTCAGTCCCATCCCCTGACTTCCGGGGAGGGGAG
AGGAGCT

| | |
|---|---|
| Sequence 973 | cMhvSB093e05 |

ACTTTTTTTTTTTTTTTTTNTTTNNGNANTATTTNTTTTTTTNTTATNTTTTTTTTCAAAGGTTTTTATT
NTATCTANNTTTNCTTNGATTGTTANACANTNGGCATNCNNANAACAACTACAANNACCACTCCTC
CGTGCTGGACTCCAACNGCTCCTTCTNGCTCTACAGCAAGCTCACCGNGGACAAGAGCAGGTGGC
ANCANGGGAACNTCTTCTCATGCTCCATGATGCATGANGGNCTGCACAACCACTACACGCANAAN
AACCTATCCCTGTCTCCGGGTAAATGAGTGCGA

| | |
|---|---|
| Sequence 974 | cMhvSB095h05 |

CNNATTGGAGCTCCCCGCGGTGGCGGCCGAGGTACGCGGGATCATTGATCAAGTTCAGAGGCTCT
GATTTGAAACGTGCATGCTTGAATACGCCATGGAGGAGCTGGTGGACGAGGGGCTGGTGAAAGCC
CTTGGGGTCTCAAATTTCAACCACTTCCAGATCGAGAGGCTCTTGAACAAACCTGGACTGAAATAT
AAACCAGTGACTAACCAGGTTGAGTGTCACCCATACCTCACGCAGGAGAAACTGATCCAGT

| | |
|---|---|
| Sequence 975 | cMhvSB096b06 |

CTGATTGGAGCTCCCCGCGGTGGCGTTGATTCTCATAGAAATAGGCACAGTCAATGTGGCGATATT
CTGCATCAATGGCCACCTTCACCGCTTCTTTCACTTTGCCGAGAAGAGACCTCCAAGTGCCCAGGC
CCACAATGGGCATCTTGGCTTTTGTACTGAGCTCCACAAACGTGGCCATGGTTGGTGCGGAAATGA
TTCTGAGTGAGCGGGTAGAAGTCTCACGTCCTGCTGTGTCCAGAGTTGTTCCTTCCAGAGGGTTCG
TGGTCTCGCTGGCTTCAAGAATGAAGCCGTGGACCTTCACAGTGTGTGTTACAGCTGTTAAAGATG
TTGTGTCTGGAGTTTGTTCCTTCAGATGTGTCTGGAGTTTCTCCCTTCTGGTGGGTTTGTGGTGTCGC
TGACTTCAAGAATGAAGCCCGCAGACTGTCGTGGTGATCGTTGTAGCTCTTAAAGGTGGTGTGGAC
CCA

| | |
|---|---|
| Sequence 976 | cMhvSB096d07 |

TAGGGCNAATTGGAGCTCCCCGCGGTGGCGGCCGANGNACNAGGTACACTNATATGGTTTTACTC
CGGCAGTCTTCNANNANACACTGATATTGNGACTGAAGGGNTCTGCACATTTTCTACCTTCTTTAC
CTTCCAGAGTNTCTCTNNCNTATGGCTTCTTACATTTCGTCCTTGGNTTTTGAGTTGANNTTCAACA
TNNGGGGNNTCCCATTTTTCCCCTATAGATGCCANGANCTTGAATGTTTNCTGCATCACATNTCTCC
NCANNNTCTTCTGTAAANGATCCAACNCAGCCCANTNNTNCTGGNNNAAANNNACAGACACATTC
TAAAAAGCCACTGNCNCCATTTTCCGGNNTNTCGGGTGTCCCGGTGTTGNCCCTAAGGT

| | |
|---|---|
| Sequence 977 | cMhvSB098f05 |

AGGTACCGCTTTGGTGACCTCAGCGTGACCTACGAGCCCATGGCCTACATGGATGCTGCCTACTTT
GGTGAGATCAGCATCGGGACTCCACCCCAGAACTTCCTGGTCCTTTTTGACACCGGCTCCTCCAAC
TTGTGGGTGCCCTCTGTCTACTGCCAGAGCCAGGCCTGCACCAGTCACTCCCGCTTCAACCCCAGC
GAGTCGTCCACCTACTCCACCAATGGGTAGACCTTCTCCCTGCAGTATGGCAGTGGCAGCCTCACC
GGCTTCTTTGGCTATGACACCCTGACTGTCCAGAGCATCCAGGTCCCCAACCAGGAGTTCGGCTTG
AGTGAGAATGAGCCTGGTACCTGCCCG

| | |
|---|---|
| Sequence 978 | cMhvSB098f05 |

GCGTAATCATGGTCATAAGCTGTTTCCTGGTGTGGAAATTGTTATTCCGCTTCACAATTTTCACACA
ACATACGAAGCCCGGGAGCATTAAAAGTGTAAAGCCTGGGGGGTGCCTTAATGAGTGGAGCCAAC
CTCACATTAAATTGCGGTTGCGCTTCAATTGGCCCGGTTTTTCAAGTCGGGGAAAAANCTGNTCGN
GGCCCAACCTGCATTTAATTGNAATTCGGCCCAACNCCCCCGGGGGAAGAAGGCGGNTTTCGGGT
NTTTGGGGGGGGNTTTTTTTGGGTTTTTT

| | |
|---|---|
| Sequence 979 | cMhvSB099b12 |

CCGGGCAGGTACAAATCTGTTGCCAGCCTGAACACACCTGTAGGAGGTGGATGGAGACCCTGGTT
GAGAGGTCTCACCCAGCCAGTAGAAACAGGATCAGGGACCTGCTTGAAGAAGCAGTCTAGCCCCA
CTTTTGTAGAACAGCTGAGCTGTGCTGGGATACCATTTCTGCCCCTCATGGTGTTGGGTTCTCCAAA
ACCTGGAAGCTGGAACGGCTAAATTGCAGAAACAGCAAAGATGGCAAGCCTGCCCCTCTCTNTAG
TAACTCTGTCCCAGGATGCTTTCAAACCCTTGTCAACCAGAGAACATCANTGGGAGAGGGCTTGAA
AACCCCTTG

| | |
|---|---|
| Sequence 980 | cMhvSB104c04 |

CACTACTTAGGGCGAATTGGAGCTCCCCGCGGTGGCGGCCGAGGTACAAAAGCCAAGATGCCCAT
TGTGGGCCTGGGCACTTGGAGGTCTCTTCTCGGCAAAGTGAAAG

| | |
|---|---|
| Sequence 981 | cMhvSB105c08 |

GATTGGAGCTCCCCGCGGTGGCGTTGATTCTCATAGAAATAGGCACAGTCAATGTGGCGATATTCT
GCATCAATGGCCACCTTCACCGCTTCTTTCACTTTGCCGAGAAGAGACCTCCAAGTGCCCAGGCCC
ACAATGGGCATCTTGGCTTTTGTACTGAGCTCCACAAACGTGGCCATGGTTGGTGCGGAAATGATT
CTGAGTGAGCGGGTAGAAGTCTCACGTCCTGCTGTGTCCAGAGTTGGTTCCTTCCAGAGGGTTCGT
GGTCTCGCTGGCTTCAAGAATGAAGCCGTGGACCTTCACAGTGTGTGTTACAGCTGTTAAAGATGT
TGTGTCTGGAGTTTGTTCCTTCAGATGTGTCTGGAGTTTCTCCCTTCTGGTGGGTTTGTGGTGTCGCT
GACTTCAAGAATGAAGCCGCAGACTGTCGTGGTGATCGTTGTAGCTCTTAAAGGTGGTGTGGACCC
AAAG

| | |
|---|---|
| Sequence 982 | cMhvSB002g02 |

CCCTTAGCGTGGTCNCGGCCGACGTACACNNGGAGAGTGANGTGGNANAAGAAGAGTGTCTGGN
AAGNGTGCTCACTGNNTTCTTNGCTNATAATGTTNAATTGNAAGAGAGNNCGCTNAGAGCTNCTN
CAAAGGNANAACANAGCTTNTTAANTNACATTGNTANACANATTGNTGGCANNCTCTGGAATGCT
TGCATGGCTTTAATGTGGTGCCTTGCNGTGTCCTGTTTTCTNNCACATTGCCNNTNAAATNATCAAA
NGGGCNCTGATNNTTTGNNATNNNAAACACTGAAATTNATTTTNTTNTCGNGAGCTCTCACGANCC
AATCTTTNCACTCACATTCTTGGCCGCCTT

| | |
|---|---|
| Sequence 983 | cMhvSB005a07 |

CCCTTAGCGGCCCGCCCGGGCAGGTACTTTTTTTTTTTTTTTTTTTTTTTTTACCATCTCAGCAAATA
CATGGTTCTTAAAAACATACATGTCCATTTCTATGTCTCCCACAAAACATCTGAGTAATTACCTCCA
GACAATGTGTGCTAAACTTCGAGTTTTGAATATTGCTTTAAATTATTGCTACCACTTGTATATGACT
TTATTGTTTACCAAGCACTTGTATATATTACCTAGTATGTACAACAACACGGTAAAGTATGTATTTA
TCAAGAAAAAATAACCAAGATTCAGAAAAACTACGAGAATTAAATAAGGTCACTCACCTTGTAAA
CGATATAGCCAGGTTTTACAACGAGGTGCGCTCAATCACAAAGTATNTGCTTTTCCCCAATATCTT
CTTTAACTATAAACATTTATTTAATGCCCACTAATTGCCAAGAATTGNGCTAGAAACTTTCAAATTT
TG

| | |
|---|---|
| Sequence 984 | cMhvSB006h07 |

CCCTTAGCGGCCGCCCGGGCAGGNACTTTTTTTTTTTTTTTTTTTTTTTTTACCATCTCAGCAAATAC
ATGGTTCTTAAAAACATACATGTCCATTTNTATGTCTCCCACAAAACATCTGAGTAATTACCTCCA
GACAATGTGTGCTAAACTTCGAGTTTTGAATATTGCTTTAAATTATTGCTACCACTTGTATATGACT
TTATTGTTTACCAAGCACTTGTATATATTACCTAGTATGTACAACAACACGGTAAAGTATGTATTTA
TCANAAAAAATAACCAAGATTCAGAAAAACTACGAGAATTAAATAAGGTCACTCACCTTGTAAAC
GATATAGCCAGGTTTTACAACGAGGTGCGCTCAATCACAAAGTATATGCTTTTCCCCAATATCTTC
TTTAACTATAAACATTTATTTAATGCCCACTAATTGCCAAGAATTGTGCTAGAAACTTTNAAATTTT
GTCTTACTCTGGTAATTNTCATGAGGGATTACCGTATGTATCATGCTTGATAGTTTATTTTCA

| | |
|---|---|
| Sequence 985 | cMhvSB007b05 |

CCCTTGGCNGCNNGNGCCCGGNCTGGTACTGATTGGNGAAGTGATAANTGTACATGAAATCNNTA
CAATGCATGTGCAAAGATGGCANNGACACATGCNTCTCANATNATAAAAATANTACTGTGNGGAA
TNAAGAAATGNTCNTNAANNNNTAACANGGAATGNTCNNGTGCCATGGCNTNNNCCANTNNNTCT
GGTGGGGGGCC

| | |
|---|---|
| Sequence 986 | cMhvSB011e02 |

AAATGAGACTGCCTCAAAAAAAAAANAATGAAACTNTATTTTAGGCTGTTCTGGAGGATTCATTA
GTGCTCCCATTCGAATGTATTTANGANACCCGNACANGGTTGCAAAAGATGGGCTTTGTANGCCAT
TTGCATNTTGGTNAAATGGGACCCTTTCCAACAGGATCAAAACCTTTTATATTGGCCACAGAANAT
TNTTGTCTCATTTNACAAACGNGGGGACTACAACTAACTATATAGTGTAATTCTTTAAAGATTTGA
AAAAAATTGTCAAAGTAATANATATTNCATTCTTTTT

| | |
|---|---|
| Sequence 987 | cMhvSB011f05 |

CCCTTAGCGTGGTCGCGGCCGAGGTACCAGAGGGCAAGAAGCAGGGGAAGAGCCCCTGGAAGCA
CACAGAGGTGTTCTGCTCCATCCCATCCCGCTCCCTGCTCTCCCCAAGCTACTACCACAGCTTTGGA
GTCACCGAGAACTATGTCATCTTCCTTGAGCAGCCTTTCAGGTTGGATATTCTCAAGATGGCAACC
GCATACATCCGGAGAATGAGCTGGGCCTCCTGCCTGGCTTTCCACAGGGAGGAGAAGACTTATAT
CCACATNATCGACCAAAGGACCAGGCAGCCTGTGCAGACCAAGTTTTACACAGACGCCATGGTGG
TCTTCCATCACGTCAACGCCTACGAAGAGGACGGCTGCATCGTGTTTGACGTCATTGCCTACGAGG
ACAACAAGCCTCTACCAGCTCTTCTACCTGGCCAACCTGAACCAGGACTTNAAGGAGAAACTCCA
GGCTCACCTCGGTCCCCACCCTTAAGGAGGTTTGCCGTGCCCCTCCACGTGGACAAGAAATGCAGA
AGTGGGCACAAAATTTAA

| | |
|---|---|
| Sequence 988 | cMhvSB014a09 |

CCCTTNCGAGCGGCCGCCCGGNCAGGTACTGCCACTCCAAGGGCATCACCGNTACNGCCTACAGC
CCCCTGGGCTCTCCGGATAGACCTTGNGCCTAACCTGAGGACCCTTCCCTACTGGAGGATCCCAAG
ATTAAGGAGATTGCTGCAAAGCACAAAAAAACCACAGCCCAGGTTCTGATCCGTTTCNATATCCA
GAGGAATGTGACAGGGATCCCCAANNTCTATGACACCANCACACATTGTTGGAGAACATTCAGGT
CTTTGGACTTTAAATTGAAGTGGATGAGGAGAATGGCAANCANTACTTCAGCCTTCAACCANAAA
CCTGGGAGGGGCCCTTTTTGAACTTTCAAAGGGAAATNNTTCTNCATTTTNGGAAGGGACCTTTTN
CCCCTTTTGAATGGCAAGAAAATNATTTGGAGGGTTTGAAATTNTTCNCTGGGNTGAGGAATTTAC
CAC

| | |
|---|---|
| Sequence 989 | cMhvSB014g02 |

CCCTTTCGACGGCCGCCCGGGCAGGTACAGTTGAAGCTGCANAGTTTTACCAGTGGNCAATTTCTT
GTGTTTCATTTAAAGAACAGTTTCACAAAGGGGCTTTATTGTGCCATTGTGGGGGCCACGTGCCAA
TCAATAGCATGGGACAAAGTAAGTAAAGGCATGAAGAAACAAACAAGCAAATTCACGAAAACAG
AAGTGCTTAAATTAACCAAGTGACAGTTTGTGCATCAGTCTCACAATGGGCTGTCACATGAAATGA
GGGGCAGAAGAGGGTGAAGTACCTCGNCCCGCGACCCACCTAAGGGGCCGAATTTCCAGGCACAC
TTGGNCGGCCCGTTACTAGTGGATCCCGAGCTCGGGGCCAAGCTTGGG

| | |
|---|---|
| Sequence 990 | cMhvSB015d09 |

ANGNGNGNTCGAGCGGNCNTNAGATGTGATGCGATATCTGCANCAATTCGCCCTTAGCGTGGTCG
CGGCCGAGGT

| | |
|---|---|
| Sequence 991 | cMhvSB015d09 |

CTCACACTGGACACCTTTTAAAATAACAACAAGGAAAACCCAGCTNAGTCCAAACTCCATGGTGA
GTTNTCTGTGTGCAGNCCTGATCAGCACGCANAAACAGCTGGGAATCCCAGGGCTGGGGCTCCTC
CCCGCGTACCTGCCCGGGCGGCCGCTCGAAAGGGCGAATTCCAGCACACTGGCGG

| | |
|---|---|
| Sequence 992 | cMhvSB027g09 |

CCCTTTCGAGCGGCCGCCCGGGCAGGTACTTTATTTTTTTTTTTTTTTTTTCGNGAAAANNGGGGGN
AANCTTTTTTNTAAAAANNTTTNNNAAAAANNNTTTTTAAANNGGGGAAATTTTTNCANANNGGG
NAAAAAAAGGGTTTTTTNNNGGNAATTTTTTCCCCCNTTCCCAANAAAANAANCCCTTTTTTAAAN
NNNNCCCNTTTNAAAACNNNNTTNNNNCCCCAAANNANNGNAAAANTTNNNAAAAANNCNTTTTT
TTTNNNNNCCCCNANAGANAAAAAAAANNGNTTTTNTATNGNGGNNNAAATACCCCANGATTTTT
TTNNCNCNGGTNTTTTAAACNCTTNAAAAAAAAANNNCCCCCCAATAAAATTGGTNTTGGGTNGG
GANAAAAAA

| | |
|---|---|
| Sequence 993 | cMhvSB028c06 |

CCCTTTCGAGCGGCCGCCCGGGCAGGTACTTTNTTTTTTTTTTTTTTTTTTTAAAAAAANANCNTTT
NCNTTTTTNCCCCGGGCNGNNTNAAANNNCNGGGCNTAAANNANTTNNCCCCCNTAANCCNCCAA
AAGGGGGGGANTNNNNGGNGNNNNCCNCNTTCCNNGGNCAAAAANCNGNTTTTAANGNCCCNCC
CAAAANGGNTTTCCAGGGGGAAATTTNNNTACCCNGNTAATTTTAAAANNNAAATTTCNGNGNAA
AANNGACCCNAANTTTGTGNGGTTTTCCNNNCCCNTTTTTNAAANNNNTNNTNNTGTATAAANNN
CNNAAAAAAATAATNNNTTTNANAAAAAAAAA

| | |
|---|---|
| Sequence 994 | cMhvSB029a03 |

ACTNTATTNTTTTTTTTTNAATNAAGTNTGGANNAAAAAANNNNNGGNTNGTGACAANNGGANNT
TNNACCCCCCNNANNNNNNCNAGGCTNNGGNCCTGGAAGCNNNTGANNTTTNACACNGAAANNN
CCCCCANNAAACNGGGGACCACCCCCTNCNCCATGGNGTGTNTTNCCCAAAACANCTTTAANTNG
GNAGGGAAAATAAGAAAAGGGGAGGTTTGGGGAAAAAGTCATCCCCAGTCTTGAATCCCTGTGGC
CAGTGAATAAGATATACGTCCAGATAGCTCAACTTCAGGTCCTTGAGG

| | |
|---|---|
| Sequence 995 | cMhvSB030e04 |

CCCTTANCNNNGGCCNNNCCGACGTGCACNGGAGCNGGGANCCGNTCANATACNNTNNCACACC
NCNNNAACTTTGNGCTTACCCTNTNGACAAANAANCNNGCTGCTGNTGNCTCTTNGGGNNCACAC
NNCCTTTAANAGCTACANNGATNANCANGACANGGNGNGGCTTCATTCTTGAANTCNGNGACNCC
ACAAACCCANCCCAAGGGNNAAACTCCGCACCCNTNTNANAGAACAAACTCCAAANNCNNCATN
TTNTACAGNTGTAACACACACTGTGAAAGTNCACGGNTTCATTCTTGAANCCAGCNNGACCACAA
ACCCTTTGGAANGAACCAGNTCTNGACACAGCAANGACGTNANANTTCNACCTGCTCACTCNGAA
TGATTTTCGTACCAACCATGGCCACCTTTGTGGAGCTCAGTACCTGCCCGGGCGGNCGCTTTAAAG
GG

| | |
|---|---|
| Sequence 996 | cMhvSB030f11 |

CGTNCCCTGANNTNNANAAACNTNGCCATNGTTNGTGCNNAAATNATTTTTATTTATCATNTAGAA
NCCACACAAAAATTTTTTTTNNGNGTTTTTTTNTTCCAGAANNAAAGGNTCTCACNTNCTTGGNGA
ANNAAAANANCCACCNTCACAGTGTNTGTTACANTTTGNTAACNNATGGGGGGGGGGG

| | |
|---|---|
| Sequence 997 | cMhvSB031c01 |

CCCTTTCGAGCGGCCGCCCGGGCAGGTACCCGNNCTTGGNGNTNAGGGTNGAGAACNTATGAACA
TTGTGTGGGGNNGNNTGNNNTATGGACNNNGNTACNTTCNTGCNNNCAANGCNNCANTANNNTGT
CTCATANCCACACTNCTACTTGGGANCCNTTACNGANNCCTGNAAAGCGGATTGNTTTCCNGNCCN
GGCGGGANTGNAAACNACCACTGNCTCCAAACAAAGCATCAACAGCTACCTGGGGATGNGGANA
ACTCTGGTTGGCGAATTTCACGAACTGGNGGAGGNTCANTGGNCNNTCACGAACAACANACNTGN
TACTGGTNGGCNTTGTTNTTGGTCCATTCTNCTGGGACCACCACCCTGGAAGGACACTTGAGCCCT
ACTCAAGGACCCACC

| | |
|---|---|
| Sequence 998 | cMhvSB031e05 |

CCCTTTCGAGCGGCCGCCCGGGCAGGTACTTTTTTTTTTTTTTTTTTTTTNGGGGNNNNNNTTNNCC
NNNNNGGGGNAAANTNNNNNAANNANAACCNNAACCCNAAGGGGAAANNANGNAAANNTNNNC
CCCTTTTTTTTTTTTTTTGGGGGGGNTTCCCCCCCNNNNTTTTTNGGGGAAAAAANCCCNCCCAAAA
AAAAATTTNAAAAATTNCCTTTNNNCCNAAATTTTTTTNTNCCCTTTTTNCCCCNANANTTTNAANG
GGGGGGTTTNNNNNANGGGGNNNNAAANTTTTTNAAAAAAAAAAAN

| | |
|---|---|
| Sequence 999 | cMhvSB032c07 |

CCCTTAGCGTGGTCGCGGCCGAGGTACTGAGCTCCACAAACGTGGCCATGGTTGGTGCGGAAATG
ATTCTGAGTGAGCAGGTAGAAGTCTCACGTCCTGCTGTGTCCAGAGTTGGTTCCTTCCAGAGG

| | |
|---|---|
| Sequence 1000 | cMhvSB033a04 |

CCCTTAGCGTGGTCGCGGCCGAGGTACAGGTATGCCCTGGCTGCCTCCACACTTCCACCCACTCCC
AGGGAGACCAAAAGCCTTCTTACATCTCAAGGTAGGGACAAAAATGGGGACCATGATGGCTGATT
ATTCAAAATAAAACAAAAAGTATTAAGGTGAAGATTTTTTAAAATGCTGCATTACATAATTTACAT
GAAAGCAATCCTGTAACCTCCCCTTTGTGGACTCAGGAGAGAACTGGGCCGTTCTCCTGAGAGAA
GTGGGGTGGCTTTTGGGAGGGCAAGGGACTTCCTGTAACAATGCATCTCACAATATGTGGAATGA
CTATTTTAAAGNNTAACCTTGNANAGTACCTGCCCGGGCGGCCNCTNGAAAGGGCNANTTCCAGC
ANACTGGCGGCCGTTACTTAGTGGGATCCGNGCTNGGNACCAACCTTGGCGTAAATAANNGGNAA
TAGCTNNTTTCCTGGGGGGAAATTTNTTNTCCCCCNCAAANNTTTCCCCCNCAAAANANCCNAANC
CGGAANTTTTTAAAAGGNNAAAANNCCNNGGGGNCCCCTNAANGGNGNGNCNCTAACCNCCAAA
TTAAATTNGGNTNGGCCCNCNCNNGCCCNTTTTTNANGGGGGAAAAACCNCGGNGGGCCCCCTTT
TAATANAAAAAAANNCTCCNCNCCCNNGGGGGNNNNNGGNGGNAAGTTTTTTGTGGGNTTTNCCC
CCNANNTTTTTTTNTTNTNTNNNNNNNNNNNNNGNNNNNTNNGGGNNGGGGGNANAGGGNTTTTN
NTTTNTTNTANNGGGGNTTTTNNANAAAAAAAAAAA

| | |
|---|---|
| Sequence 1001 | cMhvSB045c01 |

AGGTACGCGGGGGGGATCTCAGGAGGCAGCTNTCTCGGAATATCTNCACCATGGCCTGGGCTCTG
CTCCTNCTNACCCTCCTCACTCANGGCACAGGATCCTGGGCTCAGTCTGCCCTGACTTANGCTTCCT
CCNTGTGCCTGGATCTGANTGNGACAGTTCAGCGCACTNATATTTCGGNGCTCATTGGGGACGCAG
TCAGNTGNACACTCAGGNTCAGTNTAGTACACCAGACGTGNTCTANGAGTTACCTNGCCCATGNC
CNGGTTCTGTTTACTNANCAACTANATNACATCCTCCGCGTNGCCTGCCNGGGAAATATCCGATAN
TGGAAAACNAGNTTTCATACGCGGTACNCTGTCCCNGGGTGGGNGCCCCNGTACCCAAGCTTTTTT
GTTCCCCTTTTAAGGT

| | |
|---|---|
| Sequence 1002 | cMhvSB046f03 |

CCGGGCAGGTACCNGTNTTNATNTCTNNNTNGATNACNTCCGGGGATACAATACTATCCATACTCC
NNGCCGANNTNGNTATTTGAACATGNTANGGNTGCCTCACCTGCCTAGCGGGTTGGATTTCCCATA
CCGGGCTTGGCTCCCTNATGGGCCTNCCTGTTCCCNATCAGAGGGATCTACCNTNTGCCCAGAGGC
AGTNACAGGCCAAGGGAAGCANGCAGGGCTTGATATGAAGCCTCCCTCTCAACCACTGTGGTCTC
AGCNACTGNNCCCGCTGAGGNATCTTCANTTATGGGGGNANTTTNTGGGAAAACGAGNAGGGANC
CNCCTTATTTTATTATTCACATGTCNATTTTNTNTGATTCACTNNTAAGCAAAAAGTTCGAGNNTAT
ACCAAGTGTTCNTTAAAAAAAAGTAAAAGNNGCTGTTTGGGATGCTCGAGNGGGTGCTTGGCANG
AAANACAACTGGGGAATCCNAATACTTTAATAATGGACAAAGCNGTGGCGTNGCCCTTCNAAAGG
NGNGGGGGGC

| | |
|---|---|
| Sequence 1003 | cMhvSB048g08 |

NTTTTTTTTTTTTTTTTTTTTTTTTTTTTAGNANAGACGGGGTTTCACCGTGTTGCCCAGGCTGGTCT
CGAACTCCTGAGCTCAGGCAATCTGCCCGCCTCAGCCTCCCAAAGNGCTAGGACTACAGGCTTGA
GCCACAGCACCCGGCTGACACTTTTNTTNTTGGAGCCTCAAGCAACCAGGCTCCTCCTGCCAGCCT
TTACCCTCCTGGGATGTTCTANAGGACANAGCCAGGTGACAGCCTTNTGTGGGGGAGCAAGGATC
AAGGCCTTGCTTGAAAGGGTGAAAGGGTGTGTCTCCCCTTACTTCTGGGCCTTCACACACACCTCC
TTTGCCTCGCGTNTTCACCCTGCCGACTTAAGGGGCANAGCCAGACTTTAACTANAAAGCCATATT
CTCAATAACTATGCAAGGAGGAATGCCCTCCTTGAGGGCTTGAGCCANANCCTTTCATTGGGGTAG
TCACGACAGCAAANCTATTACCTTTCCCTTTTTATTTGGCC

| | |
|---|---|
| Sequence 1004 | cMhvSB049c01 |

TGAGGGCGAATTGGAGCTCCCCGCGGTGGCGGCCGAGGTACTTTTTTTTTTTTTTTTTTTTTTTNNN
NNNNNNCCCCCCCGGGGGGGGNGGGGGNNNTTTNCCCCCCNNNCNCCNNNNTNGGNNNGGGGAC
CCCTTTTTAAGGCCCCNTTNNNGNAAANAACCCCTTTTTCCCCCNCCCCCGGGGNCCNCANNGGGG
GNCNGGGAANNCCCNTTAAAANNTTNNNGGGNNAANNTTNAANNGGGNTTTNCCCCCCCCCCGGT
NNTTTTAAAANCNAAAANNTTTTNGGGGNAAAATTTTTAAAAANAAAAANGGGNANTTNNNTTTT
TTTAAAAANNNCNNTNNTTTTTTTNAAAAAAAAAAAANTTTNNGGGGNTTCCCNGGNNTTNNNNC
AAAAACCNTAGGNAAAAAAANGNCCNTTNGCCNCCNAAGGGNNGCNANAAAAANGGGNNGGNN
NGGGTAAAAAAAAAA

| | |
|---|---|
| Sequence 1005 | cMhvSB051g12 |

GGGCGAATTGGAGCTCCCCGCGGTGGCGGCCGAGTACTTTTTTTTTTTTTTTTTTTTTTTTTTTTTTTT
TTTTTTTTTTTTTTTTTTTTTTTTTTTTTTTTTTTTTTTTTTTTGGGCCNTTTNTTTTACTTTNTTTAANN
TTCCCCCCNCNANAACNNCNCCTTTTTTTTAAACNAAAANCCNTCCNGGGTTCCNGAANGGGGGG
CNAAAAAAAAAGGAAAAGTCAAAANCNNCCGGANNGGGGGGGGGGGGAANAAANAAANCNNNT
TGNCNGGGCNNTTAAAATTGNNGGNNGCTTGGANCNCCNCCTNGTTGNCCCNNNGANTTAACCNA
ANAAANCNCNCCCCCNANTNAAAANGGNCTTNCCCCCCCCCCCCC

| | |
|---|---|
| Sequence 1006 | cMhvSB052e12 |

AGGTACTTTTTTTTTTTTTTTTTTTTTTGGGGGGTTTTTNNTTTTNTTNAAACCTTTNNAAANNNNAN
NGNNAAAAAAAANTCNTTTCCNGGNTTTNCNAAAANNANTTNGGGTTTNGGGCNTGAAATTTNAA
ANCCCCCNNNGGNNNAANNNNCCGGGNAAANNNTNCCNTTTTTTTNC

| | |
|---|---|
| Sequence 1007 | cMhvSB055e01 |

CCGGGCAGGTACAAATCAATCTAAAAGAGGTCAACATCCCAAAAGCAAATGGGCAACAAATATG
AACAATTCACAGAAAATGCCAAGCTCCTGATGCTGACCCTCCCTCATAAGAAAACTGCTAATAAA
AACTCCTGGAGAGGATGCTCACACCACCCTGGGAGGGAACACAGTGGTCTCTGGAGGAAGGCACA
GCATATGCTTTCGAGTTACCAAGGCACACAGCATTGTAGGCCAGGCATCTGGCCTACAGGATACTC
ACCCAGTCTTTACGGAGCAACTGTAAAAAACAACAACTGTTTACAATTAGCATAGTATCACCTGGA
ATCTACTTACATATCGATCCTCTCATTTCAAGAGAAGAACTTCTCCAATGCACGTCCTACCATACTG
TGGAAACTGGGAACTCATTCTGCATCTAGTTGGGATAGGAGATTAATTTCTAAACCCACAGCCCTT
ATTCTGCCCACACCCTGCCCCTGATCTACCCAAAGCATTTGCAAAGTGATGANGAGGCAGCCTNCT
GGGATAGAAACTTTTGAAGAAAAAGGCCAGTTNCAGATGGGCTGGGAA

| | |
|---|---|
| Sequence 1008 | cMhvSB055e12 |

GGAGCTCCCCGCGGTGGCGGCCGCCCGGGCAGGTACTTTTTTTTTTTTTTTTTTTTTTTTTCCTTTTT
TTTTTTTTTTTTTTTTTTTTTTTTTTTTTTTTTTTTTTTTTNGCCNTCAATTTNTTAAAANAANCNTGTT
TAGCNGGTTTNANCAATNGNNTNGNNGTTNGGGGTAAAAANNCNTAAAAANGANANGGGGGGGT
TGGCANCANNCCGAAGTNGGTTTNTNNCCATNCCCTGCANTTNTGGGNNCCAANGGNNTTGCAAA
ANGTTAAAATAAATCNCAAAGNCGGGNGGCATNNNTNAATGGNANAAACCCCNCAANATNGNNT
NANAGNTTCATCCCGTNGGGGNAAAAAAAANATTCCNTCAATTNATTTANGGGNTTTNGGAGGGG
GCCTTGNCGTTCTANGANCCNNTTGAANAANNTNNTTTGTTTTNAAGCCCTTTAAACNCTTGGGGN
TTNGNNNCGGGCTTTGGAAAAANNCNCTTTTTNCCNAAAAGGGGGGGCGGNACCCNNANCCNNCN
GTNAANACTTTGTTTGGGGNGNNGGGGGCCCCCCCCCCC

| | |
|---|---|
| Sequence 1009 | cMhvSB058a08 |

AGGTACAAAAGCCAAGATGCCCATTGTGGGCCTGGGCACTTGGAGGTCTCTTCTCGGCAAAGTGA
AAGAAGCGGTGAAGGTGGCCATTGATGCAGAATATCGCCACATTGACTGTGCCTATTTCTATGAGA
ATCAACATGAGGTGGGAGAAGCCATCCAAGAGAAGATCCAAGAGAAGGCTGTGATGCGGGAGGA
CCTGTTCATCGTCAGCAAGGTGTGGCCCACTTTCTTTGAGAGACCCCTTGTGAGGAAAGCCTTTGA
GAAGACCCTCAAGGACCTGAAGCTGAGCTATCTGGACGTCTATCTTATTCACTGGCCACAGGGATT
CAAGACTGGGGATGACTTTTTCCCCAAAGATGATAAAGGTAATATGATCAGTGGAAAAGGAACGT
TCTTGGATGCCTGGGAGGCCATGGAGGAGCTGGTGGACGAGGGGCTGGTGAAAGCCCTTGGGGTC
TCAAATTTCAACCACTTTCAAGATCCGAGAGGCTTTTTGAACCAAACCTGGGCTGGAATTTAACCN
AGTGACTTAACCNAGGTTGNAGTGTCACCCATTACCTTACCCCAGGANAAAACTGATCCCAGTTCC
CTTGCCCCNGGCCGGNTNTTAAGAACTAAGTGGGATNCCCCCCGGGCTTGCAGGGAATTCNATATC
NAAGCCTTTATTCGATACCCCTTCGACCCTCCAANGGGGGGG

| | |
|---|---|
| Sequence 1010 | cMhvSB058c02 |

TTTTTAAGGATTCAAGAGGTGATCTGGCTTTTGTGAAAGTGTACGCGGGGACGGCTTCTGCTGGCG
GCCGCNGANACGCAAAGNCTTGAGCAGCGCGGNAGGCACCATGTTCCTGACTGNGCTCCTCTGGC

| | |
|---|---|
| Sequence 1011 | cMhvSB059a06 |

NATCCAGATACTTNTGCCTGCCTTGAAGTGANGGCCTNNCACCAAANGNNCCATGNGCACCNTGC
TGNCNATGAACNGGNACTCCCNCNTNANAGNCTNNTNTNGNATCTTATNTTGGANGGCTTATCNC
ACCTNATGTNGATGNNCATAGAATTAGGCACAGNGANTGGGGCGATATTNTGGATANANGGCCAN
CTTGNCCGGTTTTTTCANTTNGCCNAGAAGAGACTGAANTGCNCAANACNNGCCCNTNACACATG
TATTNTTNTTNTAAGAGANGANACNTTGCCNTGTTGCCCAGGCTGGACTAACACTGNCAGGTNNA
AACANTNCTNCGAACTCCTGAGGNANCTGGAATTACACCACACTGAGCNNCACCATATTGGTCTT
ATCCNCAGACCACNTTGNCCTGCCCCACACAGTCCAGTTTATCCAAACNAAGGCTTNCTGGGGGNC
TTCTNTTTGCCANGGAATATCTGGNAGGATACACAGTGTANAANAATTTNTCANACCAAAAGGAA
GGAAAAGCGAATTTAATTTTATGGATNNTGCCCCTTTNGCCCTATGCTANCTNAAAAGGTCAAATT
GCCCTTTTTCATTCAAGGGTTANTTCCTGAAAATGGTCCNTCCAGGGTGGNGGGGGGGGGG

| | |
|---|---|
| Sequence 1012 | cMhvSB060b01 |

CCGGGCAGGTACAAAAGCCAAGATGCCCATTGTGGGCCTGGGCACTTGGAGGTCTCTTCTCGGCA
AAGTGAAAGAAGCGGTGAAGGTGGCCATTGATGCAGAATATCGCCACATTGACTGTGCCTATTTCT
ATGAGAATCAACATGAGGTGGGAGAAGCCATCCAAGAGAAGATCCAAGAGAAGGCTGTGATGCG
GGGGGACCTGTTCATCGTCAGCAAGGTGTGGCCCACTTTCTTTGAGAGACCCCTTGTGAGGAAAGC
CTTTGAGAAGACCCTCAAGGACCTGAAGCTGAGCTATCTGGACGTCTATCTTATTCACTGGCCACA
GGGATTCAAGACTGGGGATGACTTTTTCCCCAAAGATGATAAAGGTAATATGATCAGTGGAAAAG
GAACGTTCNTTGGATGCCTGGGAGGCCATGGAGGAGCTGGTGGACCGAAGGGGCTTNGTGAAAGC
CCTTGGGGTCTCAAATTTTCAACCCACTTNCAGATCGGAGAGGCTTNTTTGAAACAAACCTTGGAC
CTGAAAAATATTAAACCCAGGTGGACCTTAAACCCNGGGTTTGGAGTTGTTCANCCCCATTACCCT
TTAACCGCCAGGGAANAAAAACTGGATTCCANTAACCCTNCGGCCCGCTTNTNAGAAAACTNNGT
GGGNANTCCCCCCCGGGCCTGNNAAGGAAATTTTCGATNTTNCAANCCTTTNTTNGGATACCCCGT
CCNAACCCTTCGAAGGGGGGGGGGC

| | |
|---|---|
| Sequence 1013 | cMhvSB062a03 |

CCAAGATGCCCATTGTGGGCCTGGGCACTTGGAGGTCTCTTCTCGGCAAAGTGAAAGAAGCGGTG
AAGGTGGCCATTGATGCAGAATATCGCCACATTGACTGTGCCTATTTCTATGAGAATCAACATGAG
GTGGGAGAAGCCATCCAAGAGAAGATCCAAGAGAAGGCTGTGATGCGGGAGGACCTGTTCATCGT
CAGCAAGGTGTGGCCCACTTTCTTTGAGAGGCCCCTTGTGAGGAAAGCCTTTGAGAAGACCCTCAA
GGACCTGAGGCTGAGCTATCTGGACGTCTATCTTATTCACTGGCCACAGGGATTCAAGACTGGGGA
TGACTTTTTCCCCAAAGATGATAAAGGTAATNTGATCAGTGGAAAAGGAACGTTNTTGGATGCCCG
GAAGGCNTTGGAAGNANNTNTNGGCCAAGGGCTTGTTAAAACCCTTTGGGGNTTTNAAATTTTNA
CCCCTTTTCCAAANNCNGAAAGGGNTTTTGNAAANAAACCCNGGACTGAAAATTNAACCCCGNGG
GCCTTAANCCCGTTTGNGNNGTGTCCCCTTNTCNCTTNACCCCGGGGGAAAAACNGTNNTCCCCAG
CTTTNNCCCNCCCCAANGGGGGTTNTTACCCNTTTNGGGGGTNNAAAANNCCCNNNNGGGGTTTTT
CNCGANANAAAAACTTTGGGGCCCAAAACCTTNGGGGACCCCTTTCNCTTTGGTGGGGNGGGANC
CCCCAAAANTTAAGGGGAAATTNNTTTGGCNAAANCCCCAAAAAAA

| | |
|---|---|
| Sequence 1014 | cMhvSB062d12 |

CGCTCATTGAGGATCTTCATGAGGNNGTACGGTNANGTTCCGGNCAGCCANGTCCAGACGCATGA
TGGCGTGGGGGAGGGCGTNCNCCTNGNNGATNNNCNCCNTNTGNNNTNNCCAATATTGAGAANA
NNTCTCCCNNCNTGGANANNANCCNNANGCTNATANGGACANTNCGGNCTGAATGGCCACNTACC
TTGGTCTTTNTAAAACNATGGGGATNCNNAAGTCTGTAATNAATNAAGATCTCACNNTAATATATN
NTCGCTGACCTCTTAC

| | |
|---|---|
| Sequence 1015 | cMhvSB065a01 |

TGGAGCTCCCCGCGGTGGCGGCCCGAGGTACAAAAGCCAAGATGCCCATTGTGGGCCTGGGCACT
TGGAGGTCTCTTCTCGGCAAAGTGAAAGAAGCGGTGAAGGTGGCCATTGATGCAGAATATCGCCA
CATTGACTGTGCCTATTTCTATGAGAATCAACATGAGGTGGGAGAAGCCATCCAAGAGAAGATCC
AAGAGAAGGCTGTGATGCGGGAGGACCTGTTCATCGTCAGCAAGGTGTGGCCCACTTTCTTTGAG
AGACCCCTTGTGAGGAAAGCCTTTGAGAAGACCCTCAAGGACCTGAAGCTGAGCTATCTGGACGT
CTATCTTATTCACTGCCACAGGGATTCAAGGTTTGAGTGACTCCCTTTCTCAGCCTCTANTTTCTGA
GCTGTTGCAGGAATTC

| | |
|---|---|
| Sequence 1016 | cMhvSB073e03 |

AGGTACTTTTTTTTTTTTTTTTGGTTTTTTTGGAAANANCNNCCCGGGNNGGGAAGGGGNAANTTN
NCCCCCNNGNNCCNTNNTTNGANNGGGGAACCNTTTTTNAAGNNNCCTTTTCGNAAANAAANCCT
TANTNCCCCTNNCCCNNGGGNNNCANNGGNGGGNNNGGAAANNNCANTAAAANNTTAATGGGNA
AAACTTTAAANNGGNTTTTCCCCC

| | |
|---|---|
| Sequence 1017 | cMhvSB077c04 |

GTGTTTCTGGTAAANCANACANNGCTCCGGGGANTANGCANNTANANACANAAAAACAAAAAGN
CNNANGNNNGANAAAAAANAAANNTTAAGGNTANANTAANACTAAAAAAAAAANATTGGGGAN
CTCCCCCTGTAACNTGAAANANAAAATGAATGCGGGNCGTNCCCCGTNAACTCNCACATTNCAAC
TAATNNTGGNNACGAAAAATCACATTGAACCCNGGANACGGACGTTTCATTGANCCGAAAT

| | |
|---|---|
| Sequence 1018 | cMhvSB077e06 |

TCAAGCTGGAGGTCATTACACCTACTCTGAGAATCGTGTGGAAAAAGACGGCCTGATTCTTACAAG
CCGGGGGCCTGGGACCAGCTTCGAGTTTGCGCTTGCAATTGTTGAAGCCCTGAATGGCAAGGAGG
TGGCGGCTCAAGTGAAGGCTCCACTTGTTCTTAAAGACTAGAGCAGCGAACTGCGACGATCACTTA
GAGAAACAGGCCGTTAGGAATCCATTCTCACTGTGTTCGCTCTAAACAAAACAGTGGTAGGTTAAT
GTGTTCAGAAGTCGCTGTCCTTACTACTTTTGCGGAAGTATGGAAGTCACAACTACACAGAGATTT
CTCAGCCTACAAATTGTGTCTATACATTTCTAAGCCTTGTTTGCAGAATAAACAGGGCATTTAGCA
AACTAAAAAAAAAAAAAAAAANTNNAAANNAAAAAAAAGGGAAANAAAAAANAANAAAAAAA
NGNTAGAAAAAAAAAAAAGGAATTTNNNNNNNGNGGGGGGGNCNCCTNTTTTTTTANAAAAAAA
ANCCCCCCCCCCCCCCCCCNGNGAGGNAAAAAAAAAAAAAAAAAANNANNNNGGGTTGTNNNNN
NNTATGNTNTGGGGGCNCNCCTNTTNGGGGGGGNAAAAAAAAAAAAAANNNCNCCCCNCNNNNN
NNNANAAAAAAAAAAAAAANTNTTTNNCCNCCNCTNTNTNNGGGGGGGGGGGCCNCCCNCCCCC
CANANNTGNNTNTTTNNNNANGNNTNTNNNCCCCCCGCCCCCCCCCNCANNAAAAAAAAANNTNT
CTTTNCCNCCTTCNAANANNAAAAAAANANNCNCCCCCNNGNGNNNNNGGGGGGGG

| | |
|---|---|
| Sequence 1019 | cMhvSB077g09 |

GTCGACCCACGCGTCCGTCCAGGTCGGTTTCTATCTACTTCAAATTCCTCCCTGTACGAAAGGACA
AGAGAAATAAGGCCTACTTCACAAAGCGCCTTCCCCCGTAAATGATATCATCTCAACTTAGTATTA
TACCCACACCCACCCAAGAACAGGGTTTAAAAAAAAAAAAAAAAAAAAGGGGNGGCCGTTAAAN
TATTTTAAAAAAAAANCNTCCCNCNCNTCCCCNNNANCNTNAANANNAAANNNANNNCNNTNGTT
NTNGTAANNTNNTTTTTNGCCCTTTNNATNGGGNNNNAAANAANNCNTTNCCTTCNNAATTTTCNN
AANNAAACCTTTTTTTNCNCNGNTTTNNATNGGGGGTTNGCCCAANCTCATAANNGTTTTTNNNNN
GGNNGGGACCCCNGGGNNCCNACCCCAAATNAATNCNTTTTCCNTTTCCTNGTTAANTNANTCGTT
GCCCTGGGCCNTTCGGTTGGGGNAANNGGTTTNANNTCCNTNAANGGGGGTATTNNGGGNTTCCC
NNNNTTTANAAAAAAAAANNAACTCTNNNNNNNNGNGNNNNNNANNAANNGGGNNNNNCCCNN
GGGGGGGNGNGTTTTTT

| | |
|---|---|
| Sequence 1020 | cMhvSB084b11 |

CTCCCTGCTATCATTNGGATTCNTTAAAAATTAAATCATCTCATAAGCTTACAAATGTTGATTTTTA
TTTATTTTTTTCATGATAAAACTTTCATATTTCCATGGNGNATGGAACTATAATTTTTTATGNGTTTC
TTTACGTGTAAGGNGAGAGTGGCAAGAACATAAAACCTTCACCTGTTAGTCTTAGATTTTCTTGGG
CTGGGGAGGGGCAGNAGGGCTGGAACCAATCACTGATGGGCNCCCAGNCCCTGGACTGAAATTTC
CNGGGAANGCTTAANCAAACTNTGTGGGGGGGGNCCCTTNAGAAATNGNCCCCCCNGCAAACAC
NAGGNCNCCCCGGGNGCCCCTNANAAACCCCCCCTAAAAGGCCCCCCCAAAAGGGGNTTTTCTTT
TTTAAAAAAACCCCCCACNGGGNGGNNGCTTNNNNAAANNNAAGGGNGNATAAAAAANNNNCCC
CCNGGGGGNAAAAAAAAAAAAANACCCCCCCCCCCCNGNGAGGGNGGGNGGGGGGGGNCTNNA
NCAAANCNCCCCCCCGGNNANANAANAANCCCACCCCCCCNCNCGCCNNGGGGGGGGGNNNANN
CCCCCCCCCCCCCNCNAAAAAAAAAAAAAAAANNCCNACCCCCCNCCNCCCNNAAAAAAAAAAA
AANANNGCCCCCCCCCCCCCCCNCGGNACANNNNANTAAAANNNNNTNCNCNCCCCCCCCCCCCC
CNCCGCG

| | |
|---|---|
| Sequence 1021 | cMhvSB086b02 |

TNCGGGCAGGTTCGCGGGGGATTAATGGGTTATCACAGGAATGGGACTGGTGGCTTTATAAGAAG
AGGAAAAGAGAACTGAGCTAGCATGCCCAGCCCACAGAGAGCCTCCACTAGAGTGATGCTAAGTG
GAAATGTGAGGTGCAGCTGCCACAGAGGGCCCCCACCAGGGAAATGTCTAGTGTCTAGTGGATCC
AGGCCACAGGAGAGAGTGCCTTGTGGAGCGCTGGGAGCAGGACCTGACCACCACCAGGACCCCA
GAACTGTGGAGTCAGTTGGCAGCATGCAGCGCCCCCTTGGGAAAGCTTTAGGCACCAGCCTGCAA
CCCATTCGAGCAGCCACGTAGGCTGCACCCANCAAAAGCCACAGGGCCCGGGGCTACCTGAGGCC
TTTGGGGGGGCCCAATTCCCTGCTTCCAAGTGGTTGTNCCGTGGAGGGCAAGCNACCACGNAAAG
TTNAAAAAGTAAGATTTNTTTNTTTTTTCCCACCANGANTACCTTTTTTTNTTCTTCCCCATTGACCC
NTTTTAACNAGCAAATTTNGGNTTTCNATTTNCCCCNTCNACCTTTTCCCAAGGCCTTGANTTTTTG
ANGGGAAAAACTTTTTTAAAGTAAAAAAA

| | |
|---|---|
| Sequence 1022 | cMhvSB090b09 |

AGGTACTTTTTTTTTTTTTTTTTTTTTNAAAAAAAAATTTNNTTTTTTNNNNNANNNNNGNTNNNNN
GGGCCNTTTTTNGNCNNANNTNAANNTTNNCCNNNNGGNTNANCCCCNNTTTNAANCCNAANCCC
CCCNAANNANNGNAAANAAAAAANCCTTNNNNNNGGNCNGGTTNNTTTTTNGGTTTTTNAAAAA

| | |
|---|---|
| Sequence 1023 | cMhvSB092g03 |

CGGTGGCGGCCGCCCNGGCANGAACTTNTTTTTTTTTTTNTTTGAANGGNATANNNNTNTTATNGA
TACNNNCGAACTNGNGGGNGGGCCCCGAACCCGGGTNNAGGGCCNTNNAATGAGTGTTTAATNN
NNGCGCTTGGCGGTANTCAAAAAATANNTGTTTTCTGAAAAAAAAAAAAANCCNNTCCNNNAAAA
CCCNNCCNGNNGGCNTTNNNNCCGGNAAANNAAANNTTTGGGGGGGGNNTTTTNNNGNNNANNT
GNGGGNNCNNAACTTTTAAAAAACCNTNTTTNNGGGGGGGNTTNTTTTTAAAAAAAGGAACCCCN
TTGNCCTTGGGGAAAAAAAAAA

| | |
|---|---|
| Sequence 1024 | cMhvSB098a01 |

ACAAAAGCCAAGATGCCCATTGTGGGCCTGGGCACTTGGAGGTCTCTTCTCGGCAAAGTGAAAGA
AGCGGTGAAGGTGGCCATTGATGCAGAATATCGCCACATTGACTGTGCCTATTTCTATGAGAATCA
ACATGAGGTGGGAGAAGCCATCCAAGAGAAGATCCAAGAGAAGGCTGTGATGCGGGAGGACCTG
TTCATCGTCAGCAAGGTGTGGCCCACTTTCTTTGAGAGACCCCTTGTGAGGAAAGCCTTTGAGAAG
ACCCTCAAGGACCTGAAGCTGAGCTATCTGGACGTCTATCTTATTCACTGGCCACAGGGATTCAAG
ACTGGGGATGACTTTTTCCCCAAAGATGATAAAGGTAATATGATCAGTGGAGAAGGAACGTTCTT
GGATGCCTGGGAGGCCATGGAGGAACTGGTGGACGAGGGGCTGGTGAAAGCCCTTGGGGTCTCAA
ATTTCAACCACTTTCCCAGATCGAAGAGGCTCTTTGAACAAACCTGGACTGAAATATTAAAACCAA
GTNGACTTAACCCAGGTTGAGNTNTNACCCANTACCTTAACGCCAGGAANAAAACTTGGNTCCCA
GTTANCCTGCCCCCGGGGCCGGCNNCGTTTTTANGAAACTTAGGTGGGAATCCCCCCCGGGCCTTC
TNNNAAATTTCCGANANTTNAAGGCTTTTNNGATNACCNGGNTAACCCTTTNANGGGGGGGNCNC
CNNNGTNCCCCNATCNTTTTTNTTNCCTTNTANCNGANGGGNTAANNTNCCCCCTTTGGNAAAAAA
NNTNNGGNCNTTNNCTTNTTTNCCTGGNGNTNAAAATTGTTTTNTCCNTTTAAAAATTTGNANNCC
CCCCCCCCCCCN

| | |
|---|---|
| Sequence 1025 | cMhvSB098d11 |

TTTCCCTGCTTTTAAATATATTATTCATTGACGGTAGAGGAAAAGAAAAGGCNNTGNGCCTNCTTG
CTNAGTCANNGCCCAGAGCACTGGGCAAACNANTTTTTCACCTTTTGCCTGGCGCCAANGAANGG
AAATGTTTGGCTTTTACATGACAATTTGNTTGGTNTNACGGTGAAAAAAACCTTTTCTTTAGGAAA
AGGAGGCCATTTCTTTTGAGGAAAANTANAANTTTAGAATTTGGGGTTATAANTTNTTTGNGGTTA
ATAAAAATTGGTTANGGGGGGGGTACAAAACAANTATTCTTGGTNCTTTCCCAATTTTNCCTCCAA
CCTTATTATNAATTCNCCACCCCCCTTTTTTTCCCCCTTGTTTCCCTTTTTAAAAAATTTTTAANGAA
TAAATTTTTGGGGAATTTTTTNAAAAAANGTNNTTTCCTTTTTTCCTTTTTTT

| | |
|---|---|
| Sequence 1026 | cMhvSB101a12 |

AGGTACTTTTTTTTTTTTTTTTTTTTTTTAANGGGNNNGGNTTTTNNGGGCCNNNNNNNNNNNNGGG
GNNGGGCCCCCCNAANGGGNNCCGGGNNNNNNAANNGTTTTTTNNNNNNNNNNNTGGGNCCCNA
AAAANNANTTNNNNTTTTNAAAAAAAAAAAAANCCCNNNCCNAAAAANCCNNCCGGNNNGCNTT
TTNCCCGGNAAANNAAAANNTTTGGGGGGGGNNTTTTTTNGNNNANNNGGGGGNNCNTAANNTTT
AAAAACCCCNNTTCCNGGGGGGGNNTTTTTTTAAAAAAANNNACCCCNTTGNNCCNTTGGGAAAA
AAAAAA

| | |
|---|---|
| Sequence 1027 | cMhvSB103a03 |

TTGAACAAGCCGGTTGACGTCCAGTTCAAGGTAACGCTCGCCGCGGCGCATGGCCTCGGGGTTACC
GAACAGGAACAGAATACGGGTGCGGGGCTTGATCTCCCACGGGCAATGCCTTGCAGCAAGCGGTC
GGCCAATTCGATCGGCGCGGTTTCGTTGCCATGGATGCCNGACGACAGCANCACGTCGNTGCGTTG
TCCCGCGCCTNAAGAGGCCGCACTTCAGCGCGCCTTACTTGAGCCAAGCGCAGTTGCACCCCCGTN
NACAGTNANTTTGAATTTTTTTGCCCCCGTTCCNCGACCGGGCGAAGGGGTTAATTTCAANCCATT
TTTGCCCGNGGGGCGAAACATAAAAACAAATTTTTTTTTTGTNGGTTGCNANNCCAAANAACCGG
GGGACANTAAATCNNNNNTAAATAAANANTTAAAAAGGGGGGGNGTTNTTANAAAAAAANNANT
GGNCCCCCCCCGGGGGGNGGGNNGNAAATNNNAAATTTNTTTNTTTTNNCNNCCCCCCNTNGGGG
GGGGGGGGGGGGGGCCCNCCCNANTTTTTTTTTTTTTTTTTTAATAAAAAAANNANGNCCCCCCCC
CCCCAA

| | |
|---|---|
| Sequence 1028 | cMhvSB105g04 |

AGGTACCCGGNGNNNCCNNCATGGNCNNGGNCTNGAATTNCGCATNAGCANCTGNNTATNGANA
TACCTANGCCGGNAGAGGGANAACACANNTGGANAAAATCNGCAGNTGAAACNGCCTTGNCCGG
ACTTAACACTCANGCCTGTGAATCNGGAAATNCNAAGACCTCCAAAAAAGGACCANTTCCTNGGA
TGTGCCCCCTCACAGAGAGATGAANGGGCACCAGAAAACATCTGAAACGGAAGAGGGGACAGNG
CNTATTCAAGAANGTGCANNGGCTACTGGGGAAGACCCANCCAGTGNGGCTATTGCCAGCATCCA
GTCATCTGCCACCTTCCCTGACCCCAACGTCGAGTGATGTACCTGCCCG

| | |
|---|---|
| Sequence 1029 | cMhvSB020e08 |

GTATGCTTGAAACAACAACAGCTNTCATNGAATATTCAGAGAGTCCACTAGGTGCCAGGCAATGT
CTGAAGC

| | |
|---|---|
| Sequence 1030 | cMhvSB021e12 |

TGCAGAATTCGCCCTTTCGAGCGGCCCGCCCGGGAGGCTAAGGGAGGCTATGGGAGGCTAAGGGA
GGCTCANGTAAGGAGGATCTCTTGAGCCTGGGAGGCAGAAGCTGCAGTGAACCAAAATGGCACCA
CTGCACTCCAGCCTGAGTAACAGAGTAAGACTCTGTCTCAAAAAAAG

| | |
|---|---|
| Sequence 1031 | cMhvSB024c09 |

ACTTAAANTTTTTTTTTTTTTTTCNTTNTGNNNGGGNAAAAAATTTTTTNNTTNNNANCNNNNTTTN
TTTGGGCCNTTTTAANGGGGCNANTNTTTTTTT

| | |
|---|---|
| Sequence 1032 | cMhvSB026e11 |

CCCTTTCGAGCGGCCGCCCGGGCAGGTACTTTTTTTTTTTTTTTTTTTTTTTNGGGGAANGGTTNNN
AGGGNCNNNAAAACNNNGNGGGGNNGGGCCCCNAAAANGGGNNNGGGGNNNAAAAANNNTTTT
TNNNNANANNTNTNGGNNNNNNAAAAAAANNNTNNTTTTTTAAAAAAA

| | |
|---|---|
| Sequence 1033 | cMhvSB027h04 |

TGGATATCTGCANAATTCGC

| | |
|---|---|
| Sequence 1034 | cMhvSB029c09 |

CCCTTTCGAGCGGCCGCCCGGGCAGGTACGCGGGATNCNCACATGATCACACAC

| | |
|---|---|
| Sequence 1035 | cMhvSB031g11 |

CCCTTANCNNNGGCCCNNCCGACGNNCANGAGTGCTCTTNTGCAGGCCACAGGGG

| | |
|---|---|
| Sequence 1036 | cMhvSB041e10 |

GGGCGAATTGGAGCTCCCCGCGGTGGCGGCCGAGGCACTTTTTTTTTTTTTTTT

| | |
|---|---|
| Sequence 1037 | cMhvSB051c05 |

TCCCCGCGGTGGCGGCCGAGGTACTTTTTTTTTTTTTTTTTTTTTTTTTTTGAGACAGACTTTTGCTCT
TATTGCCCAGGTTAGAGTACAGTGGCACGATCTCAGCTCACTGAAACCTCCGCCTCCCGGGTTCAA
GCAATTNTCCTGCCTCAACCTCCCAAGTAGCTGGGATACAGTTGCCTGCCACCACACCCAGCTACT
TTTTGCATTTTTAGTANAAATGGGGTTTCACCATGTTGGCCAGGCTGGTCTTGAATTCCTGACCCCA
TGATCCACCCTCCTTGGCCTCCCAAAGNGCTGGGATTACAGGCGTGAGCCACTGAGCCTGGCCAAT
TTTTATTTCTGAAACATTTATTATTAATGNGANGGGAAAATTACCCAGAATATATGTTCATTTCTTA
TAAAGTTAAGTCTTCCAAAACCTGGTTTNACAAAAAACTGAGGGTAAATTCAGGGCTCAAATATA
NAAACTTAAACTTTTCTTGGNAATCCAATTAAAAATGTANNTCTTAGCTGGGCCAGGNGGGCTCAC
CCCCTNTAATCCCAGCACTTTGGGGNGGCCCCGGGGGGG

| | |
|---|---|
| Sequence 1038 | cMhvSB058b12 |

TTGGAGCTCCACCGCGGTGGCG

| | |
|---|---|
| Sequence 1039 | cMhvSB065b03 |

ACTTTTTTTTTTTTTTTTTTTTTTTTGGGGGNACNNGTTTTTNGGGGCNNNNNCNGGGNNNGGGGGG
GGCCCCCCCNANGGGGGNNGGGGCNTNNAANNNTTTTTTNNNNNNCNNTNTGGGGNCCCAAAAA
ANNNNNNNNNTTTTTAAAAAA

| | |
|---|---|
| Sequence 1040 | cMhvSB071c08 |

CCGGGCAGGTACTTTTTTTTTTTTTTTTTTTTTTTTTAAANNNGGGGAANGNTTTNNNGGGCNNNNN
NNNGNNCNGGGGGGGGGCCCCCNNAAAGGGGNCCGGGNNNNNNAANNGNTTTTNNNNNNCNGN
NNTGGGNNCCNAAAAAANNANNNGGNNTTNNAAAAAAAA

| | |
|---|---|
| Sequence 1041 | cMhvSB073f02 |

GGAGCTCCACCGCGGNGGCGGCCGAGGTACTTTTTTTTTT

| | |
|---|---|
| Sequence 1042 | cMhvSB079a09 |

GATTGGAGCTCCCCGCGGTGGCGGCCGAGGTACTTTTTTTTTTT

| | |
|---|---|
| Sequence 1043 | cMhvSB082h09 |

AGCTCCCCGCGGTGGCGGCCGAG

| | |
|---|---|
| Sequence 1044 | cMhvSB083h06 |

GGCNAATTGGAGCTCCCCGCGGTGGCGGCCGAGGTACTTTTTTT

| | |
|---|---|
| Sequence 1045 | cMhvSB087a11 |

CGGCCGCCCGGGCAGGTACAGCTACTTTGGAGGACAGTGTGGTGGTCTCTCATAATCCTAAACATA
CTCTTAGAATATGAACCAGCAACACTGCTCCCCAGTATTTACACAGATGGGTTGAAAACTTCTGCC
CACAAAGAAATCTGCACGTGCACGTTTATGGCAGCTTTCTTTATCACTGCCAAAAACTTGGAAGGA
ACCAAGATNTCCTTCAATAAATGTCTTACTACATTCTGGTTGTTGTAACAAAATACCATACACTGC
GTANCTGAGGCAGGAGGATCACTTGA

| | |
|---|---|
| Sequence 1046 | cMhvSB092a03 |

TTGGAGCTCCCCGCGGTGGCGGCCGAGGTACTTTTTTTTTTTTTTTTTT

| | |
|---|---|
| Sequence 1047 | cMhvSB093e09 |

TGGAGCTCCCCGCGGTGGCGGC

| | |
|---|---|
| Sequence 1048 | cMhvSB094g10 |

ACTTTTTTTTTTTTTTTTTTTAAGGGGTNANGNNTTAACNGGCNATANNNNNANCNGGGGGTNGGC
CCCCACAAAGGGNNCCGGGCNNANNAANNNTTTTTTANNAACAGGNATGGGNACAAAAAAATAN
CNNNNGNTTTTAAAAAAAA

| | |
|---|---|
| Sequence 1049 | cMhvSB095f07 |

TTGGAGCTCCCCGCGGTGGCGGCCGAGGTACTTTTTTTT

| | |
|---|---|
| Sequence 1050 | cMhvSB096a12 |

TAATTGGAGCTCCCCGCGGTGGCGGCCGCCCGGGCAGGTACTTTTTTTTTTTTTTTTTTTTTTTTTTN
ANGGGNCNAAAAAAATTTNNNTGGGGGGNNNNNGGNNCNTTTTTTTTTNAAAAANTTTNGNNCCA
AANNNAAANTTTTAAA

| | |
|---|---|
| Sequence 1051 | cMhvSB104f02 |

ACACATTGAAATCTGCAACATGCTGGGACTGCAGAGAGCCTGGGCTGGGAGTCGTGAGCTCCACC
CGGCTGTTTTTATGACAGCTGGCAAA

| | |
|---|---|
| Sequence 1052 | cMhvSB031h10 |

CCCTTANCNNNGNCNNGGCCGACGTNCTNAGCTCCACAAACGTGGNCNTGGTTGGTGCGGAANTG
ATTGTGAGTGANCAGGTAA

| | |
|---|---|
| Sequence 1053 | cMhvSB038d03 |

CCCTTAGCGTGGTCGCGGCCGAGGTACTTTTTTTTTTTTTTTT

| | |
|---|---|
| Sequence 1054 | cMhvSB038h12 |

CCCTTTCGAGCGGCCGCCCGGGCAGGTACTTTTTTTTTTTT

| | |
|---|---|
| Sequence 1055 | cMhvSB094a12 |

TCCCCGCGGTGGCGGCCGAGGTACTTTCATNNNTTTTACACCTACCTTTTTCTGGGNNGGGNTNTN
GACCNCNATGATGTGNGCTCTGGAAGGCNTGAGCCAANTNTTTTNTAAACTGACTCNANGAGAAC
GCTAGGGCTACAAANNGTCTNCTGAAGATACAAAACCAGCGTGGCT

| | |
|---|---|
| Sequence 1056 | cMhvSA002a07a3 |

GCCGCCCGGGCAGGTACAGAGCTGGAGGCCCAAACAGCCAGCCAAATCTTGCTGTATTTTATCCA
CCATAGTATAATCCAGAGACTGTGGACCCCNAATTGGGATGCTTTTAAAATCCAAAGTAGTTCTGT
ATACACATTTGAAGAAAAATGCTGTTGAAGAAATGTATCCATAAAACACTTCAGGTCAAAAAGCA
AAAGAATATCAAGAAAAAGTTTAAATAACATGATTCCTACTGGNTTTAGATCATAATTATCATCCT
ATATTATTTATATTCGGATCACTGGTATCTTTCTCTGACAAATAATTCTGAAATACAATACATTTTA
AAGTTATGCAGGATTTTAAAGACCTCGTCTTCAAGCAAATACCAGAAGTTTAATAACAAACTTTAA
ATAAATGCTCATTTAAATAAAAGTTTATNTTTCTCCTGGCCAAATATTTGGNGNATTCTTACAAAG
ATACTTTCAATGATTAGATTCCTTANCTTAAAAAAAAAA

| | |
|---|---|
| Sequence 1057 | cMhvSA002a07a4 |

CCCTTAGCGTGGTCGCGGCCGAGGTACAGGCGGAGGGGGCAGAAACTGACATCATGGAGTGTCAG
GCACGGTGCTGGTGCTATGCATACACTCAACAAGGGCCTGGGTAATGCAACATGGAGAAGGGAAA
ACTGGGGGGCAGAACAATTTTGTCGTCTGAAAGCCTTTCACAGAGAGGCCCTGAACCCATAGCTCT
CCTTCTCTGAGGACAGAAAAGGAGGAAGTGTGTCTGTCCTGCAGTATGTGGGATGGATAGATGGA
TGCNAAATTAAGCACTGAAGTGGGTTGCTTGGAGAGGCAATGACTGCCCCTGCCCTCACCTGAAA
ATCCTTAAAGACAGAAGGGATCATCCGCCCAGGAAGCTGAGGCTGCAGGATAAGCTGGC

| | |
|---|---|
| Sequence 1058 | cMhvSA002b04a4 |

CCCTTTCGAGCGGCCGCCCGGGCAGGTACAACCCTACCACTACTCTACATCATGGAAGTCTTAACG
ATTTAGGGTAATACGATAATGAGAATACCAATATGGATCTATTAAATGAGGAGCTGAGTAAGCTC
CAAATTTCCCTCTAGATTGGTAAGTCTATAATTTATTATATGAAATTCCTAATTATTACCATACTAA
GTTCAAAAGATTTTAACCCAAATCCTTTAGTAACTGATAAACCTCATTCTTAAGATTCTTGACAGA
AATAATCTTGATGAGCTTCTTCTCTTCATGATCTTTCCAATGCTGTTATAATTTTGAGGGAATTACT
CTTATTTTCATTAATTCTGTTGCAAGGAGGAA

| | |
|---|---|
| Sequence 1059 | cMhvSA002b09a3 |

CCCTTAGCGTGGTCGCGGCCGAGGTACTCCAGCTATCAAAGGAGAATAGCCTTTAAAACACCAGG
ATCCTGGTCGAGATGGTAGAGGTGGTCTGTTTGAATTTGGGTGAATAGAGGAAATGCCAGTTAAG
GGATAGCCATTCTACAGACAAAAATGCANCCGTCTATACTTTTACTCCGTGGTAATACATTATTTG
TATTTCTTCTTTCTTAAGCCTCTTGTCTGTTTGTCTTAAGNATTTGGCTTATGTATTTGTCACCTACA
TAAAATATGCTCACTAAAACGCCACTGACTTTAAGGAATTTTAAGTATGATTATATGTGGNCCTTG
TAGAAAAACCATCTTTAAAGNGTAAAAAAANAAGTTTTTTTAAAAAGCTAAATTAGAAACCAAAA
AAGATCTGAAAACTCTGGAATGNATACATATAGAAATGGGNTTTTTTGAGGACCNTATGCTCCTCT
TTGGGATANAAATGNGTCGAAAAGAGCAAATATCTTGNAAAAATCAACTACCAAGAATACCATCN
ANGTAATGCNATNTCNAAGCCCGTTCANTNCAANANAAAAAATTTTGGAGNTAACCCNAGCCNGT
GGGGNCCCATCCNAGANTCCCTTTNTTNTGGNAACGGGNGNANNAAAAATTNCNANAATGNCTGT
GGCCCCCCCGGNGTGTNGTGGGGGGGNCTCCNGGGNNTGGGGNNANNACCCCCCNTGGGAATTTT
TTNTNTT

| | |
|---|---|
| Sequence 1060 | cMhvSA002b10a4 |

ACTTTGCTACACGGCCGGGGGCCATTGAGACTGCCATGGAAGACTTGAAAGGTCACGTAGCTGAG
ACTTCTGGAGAGACCATTCAAGGCTTCTGGCTCTTGACAAAGATAGACCACTGGAACAATGAGAA
GGAGAGAATTCTACTGGTCACAGACAAGACTCTCTTGATCTGCAAATACGACTTCATCATGCTGAG
TTGTGTGCAGCTGCAGCGGATTCCTCTGAGCGCTGTCTATCGCATCTGCCTGGGCAAGTTCACCTTC
CCTGGGATGTCCCTGGACAAGAGACAAGGAGAAGGCCTTAGGATCTACTGGGGGAGT

| | |
|---|---|
| Sequence 1061 | cMhvSA002b11a4 |

GGTACAGTAGAATCTCTCTGAACTGACTNTGACAGATTTTTCTTTTTTCCCCCTATAGAAGTGCCAA
GAATGAGAAGGCTATTTTCTAATATGCCCACATGTGCATTTGTTGCATGTGTATGAANAGGGAAGA
CAGCTTCTTTGCTTAGCAAACCACTGGTTGTATGGGATGTAAACCCATGCTTATTAATGTAATTACA
TAATATTACATAAACTGACAAAATATGAATGTGAAAGCTATTTCAATGAGACTAANTCAATGCCA
ACTAATTAAAGGTTAAGTTTCTAAAAGAAAAAAAACTCACTCATATTAGGTATGTGTGACAGTTTT
AAAAGATTAAATAATAAAAATA

| | |
|---|---|
| Sequence 1062 | cMhvSA002c03a3 |

GTCGACCCACGCGTCCGNTTACATATAATGCAACTTATATGTAAGTTTCATCAACACAGANTGAGT
ATATAAGTTGGCTAAAAGNAGGNANTACCCATCTAACAGTACAATGCTGTCAGAGACCCAGGCTC
TTTCTGGCTTATTGTAATTCATTTCCTTAGCATGTTGGGTTTTATCTTCATTCTGTTCCCTTCACAGT
TGTGGAATTCCTGTTGCAGCTTCATTTTTTAAGGACACAAGGCAGGAAAGGGGAAGGGCAACTCC
ACACCGTGTCTGTCTTCTTATCTTTGAAATTGCAAAGCTGTCCCAGTTACCTTACCACCCTACCTTG
CTTCTCTAGCAGATTTCTCTTCCATAATTATTTAAAGCCCACCTGGGGGTCACTCCAGGGTTTANCA
AAAGGGTTANCGGTTATATTTGAAAACCTTTNGAAAATTNCANCCCCTCCCATAAGTAAAAAGAA
AGGGGCCAAGGGGGANGAAAAACGGGTGTTTNTGGTTTTAAGNNCAAGGTCGTAANATTGGNTCA
AAAAGGGAAGAATAAGCCCAAGNANTANTTCNTCTTTTTTTGNNGGAGGAATAAANCCANGACCA
CCTTGTTTGCANTTTNTAAAAAACCATGGGGTNATTAAACCTTTGGGGCCNTTTTAAAGGGGCCAT
TATTTTTCCTTTTTAAAA

| | |
|---|---|
| Sequence 1063 | cMhvSA002c11a3 |

CCCTTAGCGTGGTCGCGGCCGANGTACCCCTTTGCTGTTTGTCCCCCTCCTCCCGGGTCCTGGAGTC
CGTCGTGTTCCAACAGTTTTTGCTCTTATTCCCGTGGGCTGCCTGGGCCTCCTTTCACCCGTGAGAC
TTGGAGCGGCCCCTGGGGTCTTGGGTGTGCAGCACGGATCACGCGAGACCCCTGAGACCTCAAAT
CATCTAACGTGAAGCCACAGACATCTTGGGCAATTTTAATCATCAAGAAAGAAATATGTCATTAAG
AAATAGCAGGGTATTTTGAAAGAGTTGGAAAACATCATGAATTTGAATACTTCAAGTAATACTGGT
GATACCCAAAGGTTGAAGAATGCCTCATTGGATGTAAAACAAATACTTAAAAATGAAACAGAGTT
GGATATTACTGATAATCTCAGGAAGAAACTCCATTGGGCTAAAAAAGAAAAGTTAGAAATAACAA
CCAA

| | |
|---|---|
| Sequence 1064 | cMhvSA002c11a4 |

GGTACCCCTTTGCTGTTTGTCCCCCTCCTCCCGGGTCCTGGAGTCCGTCGTGTTCCAACAGTTTTTG
CTCTTATTCCCGTGGGCTGCCTGGGCCTCCTTTCACCCGTGAGACTTGGAGCGGCCCCTGGGGTCTT
GGGTGTGCAGCACGGATCACGCGAGACCCCTGAGACCTCAAATCATCTAACGTGAAGCCACAGAC
ATCTTGGGCAATTTTAATCATCAAGAAAGAAATATGTCATTAAAAAATAGCAGGGTATTTTGAAAG
AGTTGGAAAACATCATGAATTTGAATACTTCAAGTAATACTGGTGATACCCAAAAGGTTGAAGAA
TGCCTCATTGGATGTAAAACAAATACTTAAAA

| | |
|---|---|
| Sequence 1065 | cMhvSA002e02a3 |

AGANACTTGAACAATTGGTTTATTTCTAAAAAGGGTGACATTTATAAGTATTCATGCAGCATTTGA
GTCCCTATTGGTGAGTGAGCAGACTATCCAATACTCATTGGCCCTCTGGCACAACAAAATTAAAAC
AAATAAACAAAAATCCGTGACTACCTAGGGTTGCTAGGATTGCTTAAGAAGAGTCTAAAGTTCTGT
TATACATGTGAACGCAGAGGACCCACATGCCGAGCTATTGTTTCTTTGG

| | |
|---|---|
| Sequence 1066 | cMhvSA002e03a3 |

TTTGTCTTCCATCCCTAATCCTTGATCAATCCAATCATTCATTTTGTCTCTTCTTACACAGCCTGTAG
AAAGAAAAAGACTGCATAACACTGAAGAAGTGTGGTTACAAAGTTACGACTTCCTGGCTGGGCGC
AGTAGCTCACGCCTGTAATCCCAGCACTTTGGGAGGCTGAGGCAGGCGGATCACGAGGTCAGGAG
ATNGNNACCATCCTGGCTAACGGGGTGGAACCCCGTCTCTACTAAAAATACAAAAAATTAGCTGG
GTGTGGTGGCGGGTGCCTGTGGTCCCAGCTACTTGGGAGGCTGNNGCNNGAGAATNNCGTGAACC
GGGGAGGCGGAGCTTGCAGNGAGCCGAGATCGTGCCACTGCACTCCAGCCTGGGTGACAGAGCGA
GACTCTGTCTCAAAAAAGA

| | |
|---|---|
| Sequence 1067 | cMhvSA002e08a3 |

CCCTTAGCGTGGTCGCGGCCGAGGTACTCCAGCTATCAAAGGAGAATAGCCTTTAAAACACCAGG
ATCCTGGTCGAGATGGTAGAGGTGGTCTGTTTGAATTTGGGTGAATAGAGGAAATGCCAGTTAAG
GGATAGCCATTCTACAGACAAAAATGCAGCCGTCTATACTTTTACTCCGTGGTAATACATTATTTG
TATTTCTTCTTTCTTAAGCCTCTTGTCTGTTTGTCTTAGGTATTTGTCTTATGTATTTGTCACCTACAT
AAAATATGCTCACTAAAACGCCACTGACTTTAAGGAATTTTAAGTATGATTATATGTGGTCCTTGT
AGAAAAACCATCTTTAAAGTGTAAAAAAAGAAGTTTTTTTAAAAGCTAAATTAGAAACAAAAAAG
ATCTGAAAACTCTGGAATGTATACATATAGAAATGGTTTTTTGAGGACCATATGCTCCTCTTTGTA
ATAC

| | |
|---|---|
| Sequence 1068 | cMhvSA002e08a4 |

CCCTTAGCGTGGTCGCGGCCGAGGTACTCCAGCTATCAAAGGAGAATAGCCTTTAAAACACCAGG
ATCCTGGTCGAGATGGTAGAGGTGGTCTGTTTGAATTTGGGTGAATAGAGGAAATGCCAGTTAAG
GGATAGCCATTCTACAGACAAAAATGCAGCCGTCTATACTTTTACTCCGTGGTAATACATTATTTG
TATTTCTTCTTTCTTAAGCCTCTTGTCTGTTTGTCTTAGGTATTTGTCTTATGTATTTGTCACCTACAT
AAAATATGCTCACTAAAACGCCACTGACTTTAAGGAATTTTAAGTATGATTATATGTGGCCTTGTA
GAAAAACCATCTTTAAAGTGTAAAAAAAGAAGTTTTTTTAAAA

| | |
|---|---|
| Sequence 1069 | cMhvSA002f05a4 |

GGTACTCCCTCTCCCCTCCCTATCTCAGGAATGAAGCTTCTGTGTCTGCTACAAGCCTCCAATGCCA
CAATGCAAGCTGTTGAGGGGGCTCTTCTTCAACACCTATGGGCCTGAAAGATTCCAGCCACCCAAG
ATCTTCAGCCCTGAGGTTGGAAACTGACCTGGGGGCCTCAGCTTGCTGTGACTGTCACTGCCCATG
TGTTCTTCCCCATGCCTCACCTTCCTCCTCCAAGTGCGTGAAACATCAATGAACCTTGTGCTTTTGT
CGTGTGATCTGTACACCCCATC

| | |
|---|---|
| Sequence 1070 | cMhvSA002g10a4 |

CCCTTAGCGTGGTCGCGGCCGANGTACTAACATCAATAAGTCGAGAAAATTATATTAACTGAAAG
AAAACAAAATAATAGAGAATTTTATTAAACGTATTTCTAATGTTTCTCTTCATGTTTGGAGAAAAG
CTGCCACATAATTAAAACAATTCTTACCCTGTAAAACTGATTGTCTTCCAATCTCAGGAGGTTTAC
ATTAACAGGAATATAGAATAAGAAACAGGCCTATGGCCGAGCTCCGTGGCTCACGCCTGTAATCC
CAACACTTTGGG

| | |
|---|---|
| Sequence 1071 | cMhvSA002g11a3 |

CCCTTGCACTGTGACAAGCTGCACCTGACGCTCATCCTGCTCCATTATTGCCTGACCACTAAGCTG
AAAAACGGTGTAAAACCAGGCATCGTCGCTGCCTTTTACTTCCTGCCAGGTGCGGGATAAATTCAC
CCCGCTGGTTGTCACGGTACTCAGCTTTAGTCCTTTGGCNAAATGCGTGTCCAGTACACCCNTGTA
ACGCTNANTCAGCAGGCGTCCGGNAAAATTTCCGCATACCTGATTGATTNGGGAAAGCCATTGCT
GAAACTCATTATCCACTGCGGGGTTCATGGCACGTTTTCGCTCTGTGAAATGTATTTTTATTGTTGC
ATTTGTGTTGCAATAAACGAAGCTAATGAGCCTGACTATAGGAAATAAGTCTTGTCAGGCATAGA
GACATAAGCGGTTATTGTCACGATTTGCGGAGCTTGTCACAGCTGACAANAGCGAATGTCACAGC
GAAAAAAGTGACTTTTCTTGTCGCTGCGTACACTGAAATCACACTGGGTAAATAATAA

| | |
|---|---|
| Sequence 1072 | cMhvSA002h09a3 |

CCCTTGCACTGTGACAAGCTGCACCTGACGCTCATCCTGCTCCATTATTGCCTGACCACTAAGCTG
AAAAACGGTGTAAAACCAGGCATCGTCGCTGCCTTTTACTTCCTGCCAGGTGCGGGATAAATTCAC
CCCGCTGGTTGTCACGGTACTCAGCTTTAGTCCTTTGGCAAAATGCGTGTCCAGTACACCCGTGTA
ACGCTCAGTCAGCAGGCGTCCGGTAAAATTTCCGCATACCTGATTGATTTGGGAAAGCCATTGCTG
AAACTCATTATCCACTGCGGGGTTCATGGCACGTTTTCGCTCTGTGGAATGTATTTTTATTGTTGCA
TTTGTGTTGCAATAAACGAAGCTAATGAGCCTGACTATAGGAAATAAGTCTTGTCAGGCATAGAG
ACATAAGCGGTTATTGTCACCGAATTGCGGAGCTTGTCACAGCTGACAAAGCGAATGTCACAGCG
AAAAAAGTGACTCTTCTTGTCGCTGCGTACACTGAAATCACACTGGGTAAATAAT

| | |
|---|---|
| Sequence 1073 | cMhvSA002h11a3 |

CCCTTGCACTGTGACAAGCTGCACATCCATATCGCCATCAACAAGATTCACCCGACCCGAAACACC
ATCCATGAGCCGTATCGGGCCTACCGCGCCCTCGCTGACCTCTGCGCGACGCTCGAACGGGACTAC
GGGCTTGAGCGTGACAATCACGAAACGCGGCAGCGCGTTTCCGAGAACCGCGCGAACGACATGGA
GCGGCACGCGGGCGTGGAAAGCCTGGTCGGCTGGATCT

| | |
|---|---|
| Sequence 1074 | cMhvSA003a06a4 |

CACATTCTACTCTACCATTCCTTTGCCCATTTTAATTTTTTTAAGACACAGATATCCTTAAAACTTTT
TATCAGTTCTTCATCAGATTTAGGATGCAGTTAGATTTTTCTCTCACTCCATACACCAACAATAATT
GTAAATAAATTAGAAATTTAAATGTAAAGCAAGAAATCATGTAAGTCCCAGCCAAAAATTTGAAT
AAATATGTAATCTTTGTGTGAAGAAAACTTTTTAAAAACAGCAACAAAGACAGACTATTAAGGAA
TGTAAACTGAGGAAAATATTTGCAATATATGGCAGGCAAAAAGTTAGTAGATTTAACATAGAATT
TTATTTTTGTTAGGAT

| | |
|---|---|
| Sequence 1075 | cMhvSA003b01a3 |

CCCTTGCACTGTGACAAGCTGCACAACAGAGTGATTTGATTAACGTCGCCCAACTGACGGCGCAAT
ATTATGTACTGAAACCAGAAGCAGGGAATGCGGAGCACGCGGTGAAATTCGGTACTTCCGGTCAC
CGTGGCAGTGCAGCGCGCCACAGCTTTAACGAGCCGCACATTCTGGCGATCGCTCAGGCAATTGCT
GAAGAACGTGCGAAAAACGGCATCACTGGCCCTTGCTATGTGGGTAAAGATACTCACGCCCTGTC
CGAACCTGCATTCATTTCAGTTCTGGAAGTGCTGGCAGCCGAACGGCGTTGATGTCATTGTGCAGG
AAAACAATGGCTTTACCCCGACGCCTGCCATTTCCAATGCCATCCTGGTTCACAATAAAAAANGTG
GCCCGCTGGCAGACGGTATCGTGATTACACCGTCCCATAACCCGC

| | |
|---|---|
| Sequence 1076 | cMhvSA003b05a4 |

ACGCGGGACACATTCAGAGGTGAGCCCAGAGCGGGTAAAGTGGACTGGGGAGAACTTCGGAGGA
TGTTCATGTCCAGGAGCAGCCCCACGCCCTGTATGGTCGGTGTCTAGAGCCTCACAGCAACTAAGA
CCAACCCAGCTCTCAGAAGAAGGAATGTCAAAATGTCATGTTCAATTTTACATTCAGTGCCTGGAA
TCTTTTCTTCACAATTGAAATGAAATGTGCTGAAGGAGGTGAATCCATGCATTAATCTTCAGCTCA
CAAAGGAAATACTACATAAGAAGCAAGACCACAGACTCAAGACGGACATAATTGGATTTTTTTTG
CCATGGCCTGG

| | |
|---|---|
| Sequence 1077 | cMhvSA003b09a4 |

CCCTTTCGAGCGGCCGCCCGGGCAGGTACACACAGTTAACCACAAAACAGGCCTCTCTGAAAAAG
CCATTGCCATGGACTGCCAGACAGACAATGACAAGACACAGAATACCTTCTGGTGTGTGAGCCAC
GGGACATGTGAGCTTCCCCGCTGATGCTCCTCTTATATCAAAGATCACTTTCACAAGATGAGCGAC
TCAATATCTTTTATCAAACCAATGATCACCTGCAAGCTATGGTATATTTTTGCAGCTGTGTAGAGCT
ATGTGGCATGAGAATGTGGGACTTATAAATTGCTGATCCAATAAATAGACATTATGGGCAACAGT
GTCTTATCAGCTAGTGTGTACTAAGGTTTCANGAACAGTTGTTCTGACCTTACTATCCAACGAGGA
GTAAC

| | |
|---|---|
| Sequence 1078 | cMhvSA003e01a3 |

TTTCGGAGGCCGGGNTCGGCCCTGTGTGCNATGTGTTACCCNTNTCACCANATTACCATTTTGGGC
CAAGATTCTGAAAAGCCTACTAAAGCNACNACAGTAGGACCCAAGGAAATAAGCCNATAGTTATG
TAAAAAAGGCCTTATTGTAAAACAAACCCATTTTTTTTAAGGGGAGAAGCCTTAGGTATTTTAAGC
AAGTTTCCANAAGGACCCCCAAGGCCATGTTTGGAAGNGNACCANAAGAAAGGGGCCTTTCTTTG
TGGTGGAACCTTGGTCCTNGNGGGNGGAATTTTTTCCAATCTCTGGGGAAAAAGGTTCCTTGGGGA
AGNAATTTTGGGGNGGCCCCTTTTTTTTAANAAGAAAAAGGGGGGAACCAAAAAAAACCTTAAAA
GGGGGGTTAAAAGGTTGGNAAAACCTTTTTTGGGGGTTTTCCTTTAAGGGGAAAAATTGGGGNCC
AAANGGAAATTCCATGNTCNAAAAGGAAAAAGNNAATTCCACCCCCAGTNGTNGGCCCCCAAAA
CCTTTGGTTTAAGGNCCCCTTTTTTNACCAANCCAAAATTGGGTTCCAATTTAAGGCCCAAGGCCC
CCCAAAAAATTTTCCAAGGTTCCAAGGCCTTAATTTTGGGAAAATTTTAAAAAAGCCTCTTTTAAT
TTTGGGGTCCCTTAAACCTTTTTGGNCCCCA

| | |
|---|---|
| Sequence 1079 | cMhvSA003e05a3 |

CCCTTTCGAGCGGCCGCCCGGGCAGGTACGCGGGACACATTCAGAGGTGAGCCCAGAGCGGGTAA
AGTGGACTGGGGAGAACTTCGGAGGATGTTCATGTCCAGGAGCAGCCCCACGCCCTGTATGGTCG
GTGTCTAGAGCCTCACAGCAACTAAGACCAACCCAGCTCTCAGGAAGAAGGAAATGTCAAAATGT
CATGTTCAATTTTACATTCAGTTGCCTTGGAATCTTTTCTTCACAATTGNAAATGGAAATGTGGCTG
CAAGGGGAGGTTGAAATNCCATTGCNATTAAGTCNTTCAAGCTCACAAAGGGAAATTACCTACCA
TAAAGAAAGNCANAGGACCCACAGNACTCCAANGACCGGGACCATTAAAATTTGGGATTTTGTTT
TTTTTGCCCANTGNGCNCCTGGGGAAANAGAAAAAGGGTTAACNCTTNCGGGCCCGGCGGACCCA
CCGNCCTTAAAGNGGGCCGNAAANTTTCCCANGGCCACCACCTTGGGCCCGGGGCCCGNTNTAAC
CTTAAGATGGGGAATCCCCGANGNCTTCCGGGTTTANCCCAAAGGGCTTTGGGGG

| | |
|---|---|
| Sequence 1080 | cMhvSA003e11a4 |

CGCGGGGACACATTCANAGGTGAGCCCAGAGGGGGTAAAGTGGACTGGGGAGAACTTCNGAGGA
TGTTCATGTCCANGAGCAGCCCCACGCCCTGTATGGTCGGTGTCTANAGCCTCACAGCAACTAAGA
CCAACCCANCTCTCAGAAGAAGGAATGTCAAAATGTCATGTTCAATTTTACATTCAGTGCCTGGAA
TCTTTTCTTCACAATTGAAATGAAATGTGCTGAAGGAGGTGAATCCATGCATTAATCTTCAGCTCA
CAAAGGAAATACTACATAANAAGCAAGACCACAGACTCAAGACGGACATAATTGGATTTTTTTTG
CCATGGCCTGGAAA

| | |
|---|---|
| Sequence 1081 | cMhvSA003f04a3 |

ACCTTTCTTTCCAGGCCATGGCAAAAAAAATCCAATTATGTCCGTCTTGAGTCTGTGGTCTTGCTTC
TTATGTAGTATTTCCTTTGTGAGCTGAAGATTAATGCATGGATTCACCTCCTTCAGCACATTTCATT
TCAATTGTGAAGAAAAGATTCCAGGCACTGAATGTAAAATTGAACATGACATTTTGACATTCCTTC
TTCTGAGAGCTGGGTTGGTCTTAGTTGCTGTGAGGCTCTAGACACCGACCATACAGGGCGTGGGGC
TGCTCCTGGACATGAACATCCTCCGAAGTTCTCCCCAGTCCACTTTACCCGCTCTGGGCTCACCTCT
GAATGTCCCCGCGTACC

| | |
|---|---|
| Sequence 1082 | cMhvSA003f04a4 |

CCCTTTCGAGCGGCCGCCCGGGCAGGTACCTTTCTTTCCAGGCCATGGCAAAAAAAATCCAATTAT
GTCCGTCTTGAGTCTGTGGTCTTGCTTCTTATGTAGTATTTCCTTTGTGAGCTGAANATTAATGCAT
GGATTCACCTCCTTCAGCACATTTCATTTNAATTGTGAAGAAAAGATTCCAGGCACTGAATGTAAA
ATTGAACATGACATTTTGACATTCCTTCTTCTGAGAGCTGGGTTGGTCTTAGTTGCTGTGAGGCTCT
AGACACCGACCATACAGGGCGTGGGGCTGCTCCTGGACATGAACATCCTCCGAAGTTCTCCCCAGT
CCACTTTACCCGCTCT

| | |
|---|---|
| Sequence 1083 | cMhvSA003g07a4 |

CCCTTTCCAGCGGCCGCCCNGNCANGTACGCNGNGAGAGGGGGTAAAGTGGACTGGGGANAACTT
NNNANGATGTTNATNTCCAAGAACAGCCCCACNCCCTGTATGGTCNGCGTCTATANCCTTCAGCNA
CTAAAACCAACCCATCTCTCAGAAAAAGGAATGTNAAAATGTCATGTNCAATTTTACATTCAGNGC
CTGNAATCTTTTCTTCACAATTGAAATGAAATGTGCTGAAGGAGGTNAATCCATGCATTAATCTTC
AGCTTACANAGGANATCTACATAAGAANCANGACCCAGACTCAAGACTGGACATAATTGGATTTT
TTTTGCCA

| | |
|---|---|
| Sequence 1084 | cMhvSA003h01a3 |

TGCACTTCAAGAATGCCGCCAGACAGATAGATAAACTCTTCGTGACCGTGCTGTTTCACGATGCGA
ATCATACCAGGCTTAATGGCGGTGAGCAGCGGTGCGTGGCCAGGGTAGATCCCCAGTTCACCTTCG
CTACCCGTTACCTGGATTTTCTCGACCAGACCAGAGAACATTTGTTGCTCTGCGCTGACGACGTCC
AGGTGGTAAGTCATTGCCATATCACCCTCCGATTAAGGCGTTAAAGTTTTTTGGCTTTTTCCACAGC
TTCTTCGATGGAACCGACCATGTAGAACGCCTGCTCCGGCAGGTGATCGTATTCGCCTTCCATGAT
GCCTTTAAAGCCACGGATGGTGTCTTTCAGGGAGACGTATTTACCCGGAGAACCGGTGAATACTTC
TGCCACGAAGAACGGCTGGGACAGGAAGCGCTGGATCTTACGAGCACGCGCTACCACCAGTTTGT
CTTCTTCAGACAGTTCATCCATACCCANGATGGCGATGATGTCTTTCAATTCCTGATAACCGTTGCA
G

| | |
|---|---|
| Sequence 1085 | cMhvSA004a09a3 |

GGTACTCGGGGACATTCATAGGTGAGCCCAGAGCGGGTAAAGTGGACTGGGGANAACTTNGGAG
GATGTTCATGTCCAGGAGCAGCCCCACGCCCTGTATGGTCGGNGTCTAGAGCCTCACAGCAACTAA
GACCAACCCAGCTCTCAGAAGAAGGAATGTCAAAATGTCATGTTCAATTTTACATTCAGTGCCTGG
AATCTTTTCTTCACAATTGAAATGAAATGTGCTGAAGGAGGTGAATCCATGCATTAATCTTCAGCT
CACAAAGGAAATACTACATAAGAAGCAAGACCACAGACTNAAGACGGACATAATTGGATTTTTTT
TGCCATGGCCTGGAAAGAAAGGTACCTGCCCG

| | |
|---|---|
| Sequence 1086 | cMhvSA004b04a3 |

CCCTTAGCGTGGTCGCGGCCGAGGTACTGGCACANACTGCANCCTTGGTGACTCTCCCAAACACAG
GACACTGTAGGATGAAACCAGAGTGTGTGATCTCCAGTCACTANACATTGCTGAGGGTTTAAAAG
CCTGCCTGCTTGTGAATATCCTTCCGGTCTTTTTTCCTTAAGGGCAAAGCATCATCCATTCCTATTT
GGAAGTGAGGCTTGAGTTTCACCTTGAAAATGCAGCAATTTGCACCGCTATGCTGTATGCCTCTTA
TATACTACATTTATGATTGNCAGAATTTAATCCTATAGAATGCTAAAGAACCAACCTGCAAAAGGT
CTTGTCTATACCCTCCTCTCCCCCACCTCA

| | |
|---|---|
| Sequence 1087 | cMhvSA004b06a3 |

CCCTTTCCAGCGGCCGCCCNGGCNGGNACACACTAGCTGATAAGACACTGTTGCCCATAATGTCTA
TTTATTGGATCAGCAATTTATAAGTCCCACATTCTCATGCCACATAGCTCTACACAGCTGCAAAAA
TATACCATAGCTTGCAGGTGATCATTGGTTTGATAAAAGATATTGAGTCGCTCATCTTGTGAAAGT
GATCTTTGATATAAGAGGAGCATCAGCGGGGAAGCTCACATGTCCCGTGGCTCACACACCAGAAG
GTATTTGTGTCTTGTCATTGTCTGTCTGGCAGTCCATGGCAATGGCTTTTTCAGAGAGGCCTGTTTT
GTGGTTAACTGTGTGTACCTCGGCCGGACCACGCTAAGGG

| | |
|---|---|
| Sequence 1088 | cMhvSA004d06a3 |

NAGGTACTGGTCTGCCTGAAGGCTGAGGGCAGTAAAATNATTGACATTACTATAATACTGACCTCA
ATCGAGCTAACCTTTAAATTCTGAGAAACAGGTTTTCAAACAGGTTTATAGGCCAAANAGAGTCTG
GAACACCCTAAGGGCTTGGTTTTCCTGGCCAAGTAATCAGTCAAAGCTATTACTGNCACTCTGCCT
TTTCCTTGTGGCTANATAACACAGCCCAAGTGCAGTTGCCAATTTCTAATGAATACTANGTGTGGC
CTCCATTTTATCCTGTGCAAGGGGATATTGGAAATCTTTGTTCGAAGCAATATCCACGAGAGAGGN
GGCTTCATNCCTCAAAAGTTAAGGTGGATTTTAAANCAANTTNGGCTGCTTTTTAACCAAAATTAC
AGNATGGGNTATTGGANGGGCCNAATAAAATATTTAATAAGGANGNCTAAATAAATGNTTGNAAA
ANNTTTT

| | |
|---|---|
| Sequence 1089 | cMhvSA004d09a3 |

GGTACTTATGGTGTGATGCCCTCAATCTGGGATTTGCTAAGACATGCAGCAGGACAAGTCCATCCC
ACGGCATCTAAGACATCCATGGGAAATGCCCTGAGGTCTTACTTTTTGCATTTGTTTTAGCAGAAC
AGAAACTGGGAGGAGGGAGTTAAAAGAGCTGATGGAATCCTTTTCTCAGCTTCTCCAAATCTCTGA
GAAAATAATTTATTTCACATCAAATATTGGAAGTGAAAACTCAATGGACAAAAAACAAACAAAAA
AATACATGATGTCCATCAAAATGTTGACCTCTTCAAGGCATGAAATAAAAGGGAGCAAAGCNGGT
AATATTAATATACCAGAAAAGCCAGTAAGTTTTGTTTTACCGTTTATGAANACCTACTACCTCCTGT
TTTC

| | |
|---|---|
| Sequence 1090 | cMhvSA005a02a3 |

NCCCTTAGCGTGGTCGCGGCCGAGGTACCTCTCATTTGCCACTTTTCAACACTTCCTGGCAGGCAG
GCAGCATAACTGGTCCTGCTGGGTGATCCAGACCACACTCTGCAACTCTTTCTTCTGAGCCAGGCT
CCCCTACTGTCTTTTCATTTATGTCAAGGCAGGGGAAGACCTCAAAGGGCTCTTGCATCCCAGTCT
CACTTCCCAGAGAGGCACGAGGCCCTCCAGGATGTGGGGACAGGAACTTTGGGGCAAGCCGGGGT
TGTCCANAANAATACCANGAGGGCTGAATAGTAGAAAGGANAAGTCTTATTGGTGATATGTTTGC
AAACTGGGAAAAGATAGCCTNCANTGTGGAGCAAANATGCTCCTTCTTCAAAAAGGGCAAGGGCA
GCTTGGATTT

| | |
|---|---|
| Sequence 1091 | cMhvSA005a11a3 |

CCCTTAGCGTGGTCGCGGCCGAGGTACTCCCTCTCCCCTCCCTATCTCAGGAATGAAGCTTCTGTGT
CTGCTACAAGCCTCCAATGCCACAATGCAAGCTGTTGAGGGGGCTCTTCTTCAACACCTATGGGCC
TGAAAGATTCCAGCCACCCAAGATCTTCAGCCCTGAGGTTGGAAACTGACCTGGGGGCCTCAGCTT
GCTGTGACTGTCACTGCCCATGTGTTCTTCCCCATGCCTNCCTTCCTCCTCCAAGTGCGTGAAACAT
CAATGAACCTTG

| | |
|---|---|
| Sequence 1092 | cMhvSA005b11a3 |

CCCTTAGCGTGGTCGCGGCCGAGGTACGCGGGCAAACTCATTAGCAAAGCACACAAAGACCTTTG
TGATGTGGTATTGCTGAATTAAACTACTGGCAGCCCTAGAAAGGTAAAGTGTATTTGATGCTTCTG
TGCTGTTCCCTTAGCCCAGAAAGCCCTTCCAGTTTCTGTTTAGTAAAGTCCTATTCATCTTTCACTA
CTCAATGAGTCATAAGTAATCCCATTAGGAAAGCCTGTGTGATCTACCTCCTCCCTAATTTGCCAG
CTTGAGTTTGCTTCACCCCTTCATAATACTCAAGNCAATCATAATGTCTTATAATCCATCATAGCAC
CTNACACAATGA

| | |
|---|---|
| Sequence 1093 | cMhvSA005c01a3 |

CCCTTAGCGTGGTCGCGGCCGAGGACTGACTGCTACTGGTAGACCTAGGGTCAGCTTTGAGGACTG
AGGTAACCACCACAGGAAATAAGTTTTGAGGTCTGATTTTGAAACAATATTGGAAGACCATTCCTT
TGTGAGATAGAAACTTCTCCATTTTAATTTTAGTATTTTAAGCTTTTCCTACAGGTCAGTTGGGAAT
AATTTTTATTTAGGGACTCACAATCTTGAATTTTTAGCTAAATGCCTTAAGAATAAAATATTATTTA
AAAAGTATTAAAATGCTGTGATTNCAAACAGTTTCTTGTTCAAGATGAAGAATATAAAAATATACC
ACCATGTCTCGGCAACTGGAAAAGCAGATTTTAATTTTCATTCCAAAAATGGGAGACTGA

| | |
|---|---|
| Sequence 1094 | cMhvSA005c10a3 |

CCCTTAGCGTGGTCGCGGCCGAGGTACTTTGCTACACGGCCGGGGGCCATTGAGACTGCCATGGA
AGACTTGAAAGGTCACGTAGCTGAGACTTCTGGAGAGACCATTCAAGGCTTCTGGCTCTTGACAAA
GATAGACCACTGGAACAATGAGAAGGAGAGAATTCTACTGGTCACAGACAAGACTCTCTTGATCT
GCAAATACGACTTCATCATGCTGAGTTGTGTGCAGCTGCAGCGGATTCCTCTGAGCGCTGTCTATC
GCATCTGCCTGGGCAAGTTCACCTTTCCTGGGA

| | |
|---|---|
| Sequence 1095 | cMhvSA005d06a3 |

CCCTTGAGCGGCCGCCCGGGCAGGTACTGATTAATTACTGCAGTAACCTGGCAAAGAGATCTCTCA
AAAGCCCTGCAGCATCAAGGTTTTTATGAATGGCTTAGATGAGGTGGATACAGCATTCCTGACTTG
TCGAGTCTTANAAACACAAAGCTACTGCTACAAGAGTGGCCATGGGGTCCCAAAAGAGTCTTTAC
ACACATTACAAAAGGCTAAATCTAAAAGGATTCAACATAATAAGGTAAGTGGAAGTTCCGCCTGG
AACTCCCAGAAATTTAGTTGCTCACAAAAAAGCCAAAGGCCAATTCAGTCTTAATCTGATACACTA
GAAGCACAGGGTCAAAACAGGATGATCTTCCCTGTCGCTTATCCCCCG

| | |
|---|---|
| Sequence 1096 | cMhvSA005e08a3 |

NCCCTTTCGAGCGGCCGCCCGGGCAGGTACTTCTGGGTCTAATTACCAAATTGGTCCCAGGGCAGA
GAACTCTCTCTCCTGCATTGCAGGGGATGCCTAGGCAGTGTGTAGGCCTAAGCCTGANAACTACCC
AGGCCTTCCCATACTTTGGAAGCAGTTGACACTTGACTTCTTGGTTTCCATCTTTGCACTGTGCTGT
GTAGCCCTGTGTGTAAACAGCAGGCACTCATGTGCCATTGACTCAGGGTCAGAAGCACCACAGCA
TTGACTGTGTGCTCTCTGACTGAGGNGGGAACTGCGGCANCACTGGGTAACAGGTTGGACTGAAG
TTGGTCTCATTTGGAGAGTGGGGAGCAAGG

| | |
|---|---|
| Sequence 1097 | cMhvSA005f03a3 |

CCCTTAGCGTGGTCGCGGCCGAGGTACGCGGGGAGAGAACTCATGAGTTTTCCGCTTCATCGTCTG
CTTCTGTTTTCTCCATCTTAGTTTGCCCAAAGCTTGCTGGCCGCTGTGTAGGGCTGGTGAGTGGCTG
GGGCTGTCTGAGCCATGAACAACTTCAGGGCCACCATCCTCTTCTGGGCAGCGGCAGCATGGGCTA
AATCAGGCAAGCCTTCGGGAGAGATGGACGAAGTTGGAGTTCAAAAATGCAAGAATGCCTTGAAA
CTACCTGTCCTGGAAGTCCTACCTGGAGGGGGCTGGGACAATCTGCGGAATGTGGACATGGGACG
AGTTATGGAATTGACTTACTCCAACTGCAGGACAACAGAGGATGGACAGTATATCATCCCTGAT

| | |
|---|---|
| Sequence 1098 | cMhvSA005g08a3 |

CCCTTTCGAGCGGCCGCCCGGGCAGGTACCAAGTGTCCCCAAACCACCAAATTCTGAATGCCCTGA
GCTGGCTGAATGCAGACCAAAGACTGGGTGACTGACCATTGGGAAGGCACTCGACACTGTGGACA
GGTTAAACGGTTGATCCCCAGCTGTTCTGAATAAATGTCCACATGGGTTGATTGTAGAGCTAAGTG
AAGCAACTCCAGTGGAAAGGCCACCTTTTGAAACTACTGAAGCCACAGAAGGTGTCGAAGATGAA
GTTGGTGTAGTAGAGGAGGCTGCTGAGGATGGTAACCGTTCTCCAGACTCCATATTGTGATCAATG
TGGTCAATCTTGTGACATCACTTGTTGGGAAAC

| | |
|---|---|
| Sequence 1099 | cMhvSA009b08a2 |

ACTAACATCAATAAGTCGAGAAAATTATATTAACTGAAAGAAAACAAAATAATAGAGAATTTTAT
TAAACGTATTTCTAATGTTTCTCTTCATGTTTGGAGAAAAGCTGCCACATAATTAAAACAATTCTTA
CCCTGTAAAACTGATTGTCTTCCAATCTCAGGAGGTTTACATTAACAGGAATATAGAATAAGAAAC
AGGCCTATGGCCGGGCTCCGTGGCTCACGCCTGTAATCCCAACACTTTGGGATGCCGAGGCGGAC
GGATCACGAGGTCAGGAAATCCAGACCATCCTGGCTAACGCGGTAAAACCTAGTCTCTACTAAAA
ATACGAAAAAAAAAAAGGAAGGAAGGAAAAAA

| | |
|---|---|
| Sequence 1100 | cMhvSA009e06a2 |

ACACGTGGAAGTTACCCCAGTGCCTCCCACTTTAGACTACAGGTCATAACTCGGTGTGGGAGTAGA
GCCATTCCACCCATGGCCAGGAAAGCTGTGCCCAGTTACAAGTCCTGTGACGCCTTAACATAGGAA
TAGTTCTGTTTTTCAAACAAGTTGTCGAGAAGTTACCAAGAAAATAAAGAACCTTCTTCCCACAGA
AGAAGGCAGCCAGAATACCCAAGTCCTAGAAAACACTATATTGCAAAATTAGAACAAATAATAAG
ATGTCTTGGCCGGGCGCGGTGGCTCATGACTGTAATCCCAGCACTTTGGGAGGCCAAGCTGGGTGG
ATCACCTGAGACTGGGAGTTCGAGAGCAGCCTGACTAACGTGGAGAAACCCCATCTCTACTAAAA
ATACAAAACTAGCCNGGCATGGTGGCGCACGCCTATAATCCCAGCTACTCAGGGAGGCTGAAGCA
GAAAAATCACTTGAACTTGGGAGGCANAAGTTTGTGGTGAGCTGAAATCGTGCCATTTGCGCTCCA

| | |
|---|---|
| Sequence 1101 | cMhvSA010a01a3 |

CCCTTAGCGTGGTCGCGGCCGAGGTACCTGCAAAGGCACTGAGGTGGGAGGGAGCATGCCAATGT
AGGGAAATGAAGAAACCCAGTGTGTATGAGCCAAGCTGAATAAAACATGAGAAGAAGCTGGAGA
ATGAGAGAGACCAGTCCCCAAGCTCTCAAGGAGCAAGAGGAAGCCTTTTCGGCATTTGAAGTGGA
GGGATGGCATGATCTCGTGCGTAGTTTTTA

| | |
|---|---|
| Sequence 1102 | cMhvSA010a04a3 |

CCCTTTCGAGCGGCCGCCCGGGCAGGTACGCGGGTCTTTTAACTGTTATGGATGTATAAGCACTAT
CTATGATGGACGAGGCATAGTGCATCTCCTAGGCCGGAAATGTTTCACTCACTAATGAGCTGGACA
ATTCTACTCTGTGAATTTAACTTTCCTGACTCCCATATGCAGGTTAATTTTGGTAACATATCATATT
TTACTCTGGCTTGGTGGGATTAGGTGGGAAATTACAGATTGCATCAACAATTTGGTCTGCCTGGAT
ACAATTTGGTCTGTTTCAATCACAGCCTGGGTCACACCTGTTGATATATATTTTTAAACTGATTCCT
CTCTAGATCATTCTTTCTGATCAGCACAAGGCAATATGCTGAAATTTCTCTTTTATATCTGTTTTATT
A

| | |
|---|---|
| Sequence 1103 | cMhvSA010a10a3 |

ACGCGGGGAGGCTGTAGGTGGGCTCCGCTGGGTAAAAGTTGCCGCAGCAGCTGTCCCTTGGCCCC
ATCGCGATTTATTTTTCCCCCTTGCTTTCCGGGTCCCGGGATCCCAAGTTTGTAACTAACGGGAGCG
AATCCACACCCGAGCAAAATGTTTGCGAGTTTCAGGCGCCCTTAGTTGAAAGGTTGTAATTAACAA
GTCCGCTGTTTGCCAGCCAGGCGCCGTTGCAGGCGCTTTCTGTGGATTGTCATTTATTTCTTACAAG
CACCCTAGGAGGCTGTTATCCTTGACATCTGCAGCAGCCCTTCCAAGCTGTGGAGACCAGGTCATC
TGGAATGCCCATTTATGTCAATGGAAGAAAGAAAAAGGGG

| | |
|---|---|
| Sequence 1104 | cMhvSA010a12a3 |

CCCTTTCGAGCGGCCGCCCGGGCAGGTACAGCTGCTTGGCCAGGGTCCCTGGCTCTGCCTACGTCA
TCTGGGTGTGTAGCTATAATAACAAAAATGGCAAAAAGGATATTAAGTGGCCATACCTTTCTATCA
AGGAAAGCTACCCNCTGNCACAGACTCATGATACCTTTAGGATTGAAGATTCGCACATCCTGGATT
TAGCCTGTGTGCCATCAATGTTCTGTTTATTGGAAGGAAAGAAATTGATTTCCTGTTTCCTTAGTTC
ATTCATCTATTAATAAACATTTTTTAGGCACCCTACAGGTCCCAGATACTATGCTATGCAGGCAGC
AAAAACACAAATAANACATAATCCCTGCACTGAGGGTCTACTGGGGTAGTGTAGCAGGGGTGGTA
GGCAA

| | |
|---|---|
| Sequence 1105 | cMhvSA010f11a3 |

CCCTTAGCGTGGTCGCGGCCGAGGTACAGGTAGGGTTCATTTGCATTCCTGCAGGTATCCCAGAGG
GAGGGTTCTGGAGGAACTTTGAGCTGTCTAGATTACCCGATGAAAACTTGTTCTTTTATCAACGGC
CACTTCCGGAGCTCGCGCAGGGGCCGCTCACTAGACCACTGCTCCCTGCCCGTGTGCCCCAGTTCA
GAGTAATCTGTATTCTTCACAGTCCCTTCTTCCAGTGAAAGCATCTCTTTTACCTTTCACCAAGCCT
TACCTCTAAAAGGCCAGTGATACCTTAGACATTTCAGAAAGCTCAAAATGATGACTCAAAACTATA
ATAAGCAACGTGCCTGTCCCTTTACTTTTGTTCCCCTGGGAGTTATCAATTGGTCGTCTTGAAATG

| | |
|---|---|
| Sequence 1106 | cMhvSA010g02a3 |

CCCTTAGCGTGGTCGCGGCCGAGGTACTGATATAGGCTGACCTAGAGGAATGTATTTTATGAGGCC
ATTTGTTTTTTGTTATGATGCTTTCAATCCCTTTTACAANTAACTTTTTAAAGTTTCCCCTGAAACAA
GATGAGGGGACCCATTTCTCTTAAGGAGCACAGCACACTGAAAGGCTGTCAGTGGCCAGACGACC
CAGCCACACAGAAAGGCACCCACAGCAGCTGCTTTGTCTTAAAGGGAAAAATACTGGCAGATCCA
GGAGCTGAGAAAAATATCAAACGAGGAAGTATGACTGCCATTTATATCTTCCCCATGACTATGTGA
CTAGGATACTCAGCATTTTTCCTACCAAGGTAATGGCAATGGGGCAGGAGTAAGGTCACAGGGAA
GCTAAAGAGGGA

| | |
|---|---|
| Sequence 1107 | cMhvSA018a11a3 |

CCCTTNCGAGCGGCCGCCCGGGCAGGTACTTTCTTAAAATTAATAAAAACTTATCAGTAAACAATT
TCTATTCCATCAGAAAGTGAGAAAGCTNAAAGATAAATCAGTAAAATGATACTAGAAAAACAATT
ATGGCTCTCTGTGGTTCCCCGATGAGACTTACAATAATAGTGCTTTAGGATTTAGCATTAAAATTA
GATATATTAGTGTTTTATTCATCTCTAAGACAGAATAGTTAGTAATACTTATTCTGCCTTCTACACA
ATATGGTGGTGATAAAATTAAATCATGAATAAGAAAATAAGACAACTTTTATCAACTATAGATTTA
TAAACAGTGACAGCAATCCTAAATGATAAGCCATTCTGGCCATAACTCTGTATTTTACTCCTTCTTT
TGGAAGACTGAAA

| | |
|---|---|
| Sequence 1108 | cMhvSA018b03a3 |

CCCTTAGCGTGGTCGCGGCCGAGGTACACTGGAGGTAGGGAGCTCAGGGATGGCAGCTCAGATCC
GGAACAATTACAATTCAATACTTGGGCATCAGCACTCTAAATCCCGAGGAGCTAGCCAGGAGTGA
AGTGAGGAAAGAGCAAATCAATTTAAACATTGCTAAATACCAAAGACAAGCTAGCTATTTCTTAC
TTTGCATGAGGCTTGCCCACGTCCTTTCTTGTAAATTGTCTGGACCATCTCTGGTCATTTGGTGGCA
TCAGCAGGACAGAGATATAGTGAGATGCAGAGAGCCATCGAAGTTGTCTGACTTGGTGGAAACAA
ATGTGACTTGGCTTGGAGTGTCAAAGCAAGAATGAGTGCGTGCATCAGATGGAAGTTGTCCATGG
GGTCTTGCAGACATGCATCGTTG

| | |
|---|---|
| Sequence 1109 | cMhvSA018c05a3 |

ACACTGTTCTATATTTTAGCAGGGAAGGAATTTGTGTATGTGTGTGCTAACTAGAAACAATGAGAA
ATAGCTCTAATGAAAGTTATATGGTCAGAATTTGGCTACAAGCTCTGCATCATTAGTAAAGCGGAG
TATTATTGGCAGATGTCATGCTACTTTCCAAAAAGCCTGAACCCATCCTGATTTCTCCTTTCTTAGT
TGAAATGCCAACAATTGCATATTTGCTTAATTATTGCTTTTTAAAATATTGGCTCTGTATAAGCAAG
GGAAAGTAATAGAAAAAGTATTGTTCTTCCAAGTAAAGCAGAACACACCAAGTGGACAATAGCAG
CTTATATTTTCACTCAACATGGGATACTATTTTTAATAAGGATGTTTT

| | |
|---|---|
| Sequence 1110 | cMhvSA018f04a3 |

CCCTTTCGAGCGGCCGCCCGGGCAGGTACTGAACTGGGAGGTTTTTAGTCTGATAGCCACAATTTT
GACCTAGGCAGGAAGCTTTACAGCTTGAGGCAGTTTCATGGTCTGAAGACAAACTTCTTGTGACTT
GCTGCCGGTGTTGGACTGCAGGAGAGAGCCTCACTGGGTCAGGAGCACGAGAACAAAGTGGATCC
CACTACCACATCCCACCCCTCCTGTTTCAGAGGCAGATCATGGGACCAGGACTACTGAGAGTTCCA
TGGCCCTACCCATCATCTGAAATGCCCAAGAACTTCTCCGATTAACAAAGGTCAAGCATAAACTCT
ATTGCCACCACCACAGCTGGTTCTCACTTTTAGGTGCTACCTCCTGTCCTAAAGGTTGATCTACACA
GTCCCT

| | |
|---|---|
| Sequence 1111 | cMhvSA018h12a3 |

ACTGGCAGCAACCACCACTGGATGAAGGTGCTTATTGCATCTCATTCTTTGGATCTCATTTTTACCC
ATAGGCCTCTGGGGCACCATATTAAAATTCCAGAGGCCATTCCTGGCCTTGTTTCATACCTTATGG
GAAATGACGCAGGTTATATGGTATGGATCTATAGGTGTAAAGACTGGGTAGCAATGGCTGGATTG
GCCGTACC

| | |
|---|---|
| Sequence 1112 | cMhvSA031b12a4 |

CCCTTAGCGTGGTCGCGGCCGAGGTACAACGTTAGCAGCAATTCAAAAGGGCATCGGAGACAACT
AATCATTTCATAATGAGCGAGGGGAGAAGCAATAAAAGCCGGGAGCCCAAGGACGGCATGATAA
TTTTGCAGAGTCTCAGCTCTCAACCAGACTCACGTTCATAAAATAAACAAATGTTTTTGGTAATGG
AAAGCTAATGTATACATTATTTAAGGATAGTATTAAAACCAGACTAGATGGATCAAGTAATACAA
CAGTTACCTCATTAAGCATCCTTTCTTTGGGGATGTGAAAAAGTTATTCTTTTTTTTCTTCTTCTTTT
TTCCTTTTGAAATGGGGCTTTATTAATTAGAGATGTAATGGGAAATCTTATTTTTTCCCCAGACTAG
TGGCTGTTTTCTGTTTATTTTTTAATGGA

| | |
|---|---|
| Sequence 1113 | cMhvSA031c02a4 |

CCCTTAGCGTGGTCGCGGCCGAGGTACAATAATGGCTCATTGCAGCCTCAACCTCCAGGGTTCAAT
CAATCCTCCCACCTCAGTCTCCCAAGTAGTCAGGACTACAGACATACAGCACCACGGCCAGCTAA
GTAGAGACAGGGTTTCACCATGTTGCCCACGCTGGTCTCAAATTCCTATGCTCAAACGATCCGCCT
GCCTTGGCCTCCCAAAGTGCTGGGATTACAAGCATGAGCCATCATGCCCAGCTCGTAAAGATCTTA
AGTCATATAACACCCTCACTCAGCTTCCAACTGGTGATAGCTATATCATTACATACAGAATATTTG
AGTAGATGGTTACTAGGACAGCAAGATGTAAGTTGCTTTGGTTCAAATAGTGGTTTACTAGAGTTT
AATCTCAAGTGTTGGTTCTGTTT

| | |
|---|---|
| Sequence 1114 | cMhvSA031d01a4 |

CCCTTAGCGTGGTCGCGGCCGANGTACACTCTCTGCCTTANAACTACCATCCTTTGCACTACATTCC
AGATAAAGGATTTTGTTACTACATTCTAGGTAAAGGATATTGATACTATCCTCAAGTTACACAGAA
AACACTCAAGGATGTAAAATCAATATTTATCTCAAATTTGTTGACTGCTACTGCTATNTTTTTTGAA
GAATTAAAAGATAAATTAAAATTTCTAAAAATATGCCATATATCAATAATTTACAATAGCTTGATC
AGCCAAAAAATCCACCTTGAGCTTAAAGCTAGAGTTTGATAGGGGTGATCCTTACTCTCCTAATTT
AAATATCACTGTATATTAGTTTTACAATATACAGTGTATATTGTGTATATTGTGTATACAATATACA
GTGTATATTCTTTTTCCAAA

| | |
|---|---|
| Sequence 1115 | cMhvSA031g06a4 |

ACGAAGTGTGTTTCAGAGTGGCGAGGAAGGGCAAGTTGTTAAGATTGGTTGTTGAATTAGTTTCTG
TTTGATGTTAAAGAGAACATAGAGTAAATGATAATCCCTCGAAAGTGGAGATCTTGGCAGGCTGG
CGCCTGGTGGTATAGTAGAAATCTGAGAAAGGGGGAGGATATTAAGTCAGTTTTATCAGGTAAAG
TTGAATGAAATAATCAAGTTTAAGTGCGTCTTGGGTATTTGCAAAGATGTATAGATTAAGGCTAAA
AGGGTTGGAGAAATAGATTTGGGAGTTACCTATGATTTTTTTTGGTTATTCTGCTCTCAGGATTGAA
AACTAAAGAATCTCAGAACTGCATTTCTAATTAGTGCCATAAAATTCTTTATTGAT

| | |
|---|---|
| Sequence 1116 | cMhvSA031h12a4 |

GGTACGCGGGGACATTCAGAGGTGAGCCCAGAGGGGGTAAAGTGGACTGGGGAGAACTTCGGAG
GATGTTCATGTCCAGGAGCAGCCCCACGCCCTGTATGGTCGGTGTCTAGAGCCTCACAGCAACTAA
GACCAACCCAGCTCTCAGAAGAAGGAATGTCAAAATGTCATGTTCAATTTTACATTCAGTGCCTGG
AATCTTTTCTTCACAATTGAAATGAAATGTGCTGAAGGAGGTGAATCCATGCATTAATCTTCAGCT
CACAAAGGAAATACTACATAAGAAGCAAGACCACAGACTCAAGACGGACATAATTGGATTTTTTT
GCCATGGCCTGGAAAGAAAGGT

| | |
|---|---|
| Sequence 1117 | cMhvSA032b12a3 |

ACCTGCCTTGTTCATATCCAACTAATTTTTTTTGGCTAATTAAGTAATAATAATCAAAACACTTAAG
GTTTTAAAGGATGAATGACCAGTTGAGAGTTACTTCTTATTTGCTCCTAAATCCAATATATTTCCTG
ATCAGTCAATAACACTTAGAACATCTAGTTATAATTGGTAATACAATTGTTTAAAAAATGATAATT
AAAAGGACTAAGACTATATATGGTCTTTTGAGGGGATAACAATTGAATTATTTAAACAAAGTATAT
TAGGATAATAAAACACGAGAAGTCAGTCCAGTGGTTCAATCCATTATTCAGAATTTCATTCTGTTT
ATAATTAAGCAACAGTGACCTTCAGGTTAGTCTTCCTTAGCTGTTAACAACCAGCTGGAGAAGCTG
AGGGCTATTTTTGCAATTATAATCTGTGAAAGATTGAAAAACCGTTAAGATAAATAACGTGTCCAC
CTTATTAACAGGCACTCATTTNCACACTTTGAATACATATCAATANGGGTTNCAAGTTCAATTTNCT
TACCGAACTTTTTTTAACTCTTTTAANAAANCCCCTGTAGGGANGNGGNGCCTCACTGGACTCTTTT
NTGGGCATTGCAATCTAATTTCAAAAGCT

| | |
|---|---|
| Sequence 1118 | cMhvSA032d09a3 |

ACTCTGTTTCAGGCCCTCACTGGGTGCCGGAGATCCACTAGAATACAAGATCTGTTTCTGTGTCTTT
GAGGGACATGTATCCAGCAATTAGTTACATCAGTCCCTTGTAGATGTCAATTCCAGTGTCACAAAT
TTCTTGTTTTGCAACGTTGAGCAAGTTTTTTTCAATGTTTCTAAGCCTCAGTTTTTTGCCCTACAAAA
TGTGGTAATAATATTTAACCATTAGTAATGTTGTGAAAATTAAGCAAAAATACATGTAATATATTT
AACAATGCTTGGTGTTCGTTAATGCTTTAATATATGCTAACTACTTATATTATTGTTGTTGTTGTGTT
AAACATGCATAAGACAGCAGGTACC

| | |
|---|---|
| Sequence 1119 | cMhvSA032d12a3 |

NGAGCGCCGCCCGGNCAGGTACCTCTATCTTGCTCCACCATTGCTGCCTCTGATTTTTCCCTATCAA
AACAATTATGAGGTCTTTTCCGCAGACTGTGTTAGCAGTTTTTGCATCCTCTGCTCATTCCTCTGNC
TCCTTGTCTTCCTCTCCANCTCANCCCATGCCCTGTCAGTGCCGCCCAGCTCACAATTGCCTGATCC
TTGGTGGGTACC

| | |
|---|---|
| Sequence 1120 | cMhvSA032e01a3 |

CCCTTAGCGTGGTCGCGGCCGAGGTACATCTACAGAGTGGTGGGACTGGGCCAGGCCTTGAACCC
AGTGGTCTGATTCAGAGCCCATGCTCTTATTAGTGTTTCCCACAAATGGGTAGTGAAGTAAATTTC
TGATAAAATGAAAAGTTCTCTTTGTATACTGATATCCATTACAAAACCTGCAGGACTACAGCACTT
CACAAAATGCATCATTTCCACAAACAGTGATGTTCTTTTTCAGGGTAAACTATATTGCAATAACAG
CAAATATGAAAAGATACTAATATAGTATCTCACATGCCC

| | |
|---|---|
| Sequence 1121 | cMhvSA032e07a3 |

GGTACCCAGAGAGCCAGAAGGCTGTTGGTGAGATGGAGCAGTCACTGAGCGGGTCACCAGGAGA
ACTTACTTTATGAGATCTGCTGCTAATTTCTGACTTTGGGCAAGTCACCTCACCAGTCTGGGGCTAA
GATTCACTCCTCATCAGTAAAATGAATACTTTGGATGAGACGGGAGGTTTTCCCATTCTGATGCTA
GGATCTTGTTCATGAGTTAATGAAGACAGTTGAGGAAGGTAAGGAGCTATTTCTACTTGATTAGTG
AGGCTTCAGTCTATTTCAACATTTCAAAGTTTTTCATGATAATTTGTTCATGAAAAAAAAAGAAAA
CAGAGGAGTTGCTCCAGCTCTAAAAAAATTTGAAAACCACACCCTGTGCTAATTGCAAGTCTA

| | |
|---|---|
| Sequence 1122 | cMhvSA032f02a3 |

CCCTTAGCGTGGTCGCGGCCGAGGTGCAGCTGTTGTCCATGTGTAGAGCTTTTAATAACCAGCGCA
GCAGGCCCCTTCACCTGCTTTTATGCCTGGACCAGATGACTGAATGTAGAACTTTAGGCACTTTTTT
TTTTTTGAAACGGANTNTCGGTTTGTTGCCCAGGCTGGANTGCANNGGCCCAATNTCGGNTNANTG
NAAGCTNTGCCCCCCGGGTTNACCCCNTTNTTTTGCCTNANCCTCCCAAGTAGCTGGGANTACAAA
CTCCCACCACCNTGCCCGGCTAATTTTTATNTTTTTTANTAAAAACAGGGTTTNACCGNNTTACCCA
GGANGGTNTAAATNTCCTGACCNGGGGATCCNCCTGCCTTGGCCTCCCAAAGNGCTGGGATTACA
GGNGNNANCCACCAAATNGGCCNTTTAGGCCCTTTTTANTTTTAAAGGNNAAAAAACATCCTTTAA
AAAGTTAATTCC

| | |
|---|---|
| Sequence 1123 | cMhvSA033b03a3 |

CCCTTTCGAGCGGCCGCCCGGGCAGGTACTCAAGCTTTGGCTTTTCTGAACTTTCCTTATTTTCAAA
AATGTCCCCCAGCCCCACTTCCACCTGAGACATTCACACACCCCATTTCCTCTTCCAGGAAGGCTCT
TATGTCGCCTGGGTAAACTTACTCTTCAAGTCTAGTGACTTTTTTCCAGAAGCTTTCCTGATATCTT
TCCATTTCACCCCACTGCTGACTTATTAAAATTTCTAGAATTTTATACTTTTACACTACATTCTCTGT
GTTGTATTCTCTTATTCAGGGTCTGCTATTTAATTTTTAAGTTCCTTGAAAATAGAGACAATTTCAT
TGTTTTCATCAGTTTGGTCCAAGTATATATAACATAGATGAAAAATAGATATTTTGTATTAT

| | |
|---|---|
| Sequence 1124 | cMhvSA033c07a3 |

CCCTTAGCGTGGTCGCGGCCGAGGTACTTTTTGCATTTTCAAATGACTTTGACTATTGCCAGAGTCA
TTATAGACCTGCCTATGATGTAGGAGTTTATTGTATCTAGTGGAAAACATACCTGTTTGTGGGGCA
GAAGCTTCTGTTCCATTCATCCTGATTTTAGACACAGCATTTAACTTTTCAGGTTCAGTTCCATATG
TATAAAGTAGGGATAATAGTGACATCCTAGTGTATTAAGAATTAAGGTGTNATTATTTCTGTCACT
GNTACTTCACCCTAATTT

| | |
|---|---|
| Sequence 1125 | cMhvSA033c12a3 |

CCCTTTCGAGCGGCCGCCCGGGCAGGTACCACCATGCCTAGCTAATTTTTTGTATTTTTAGTAGAG
ACAGGGTTTCAGCATGTTGGCCAAGCTGGTCTCAAACTCCTGGCCTCCTGTAATCTGCCCGCCTGA
GCCTCCCAAAATGCTGGGACTATAGGAGTGAGCCACTGCGCCCAGCCTTCAAATTCATTCTTTTAC
TTCTGTAATCCTAGTTGTTTAAGAAATTTTGCAAATTCAATTAATTTTCTTTTCCCTTTCCCTCTCTC
ACTGATTTGTCACTTTCTCAATAAAGAATTCAAGGTTTGAAAAATTATTGTGGCGGCAGTATTCAA
AAAACTTTCCTTCACTAAACACACACTTAACTGTGTTCCACTACTGCTGTTGTCTATACTTTAAGGG
AA

| | |
|---|---|
| Sequence 1126 | cMhvSA033e05a3 |

CCCTTAGCGTGGTCGCGGCCGAGGTACAGGTAAGGGGGAAGTTCCAAAGCTGTTAGTCACCTTGTT
TTCATGCTGATCACCCAACCAGATCTAATGTTTGATGTTCTAAGAACTTTAATGTTTTGGAGGAAAT
ATCTTGTGGCCTTCAAAAAATCATTCTGTGAAATAGTTGTTTCTACCTACATTCGTCTCATTAATTT
TTCTACATACAGCAGAATTCTGCATATATTAGAGGTAACTCAGTCAGGGTGTCATGGAGGAAGGTG
GCCCATGGTTCACCATCTTGCCAATAGAAAAACCAATAGGAAGTCATCTAACCATCATTCGGAGG
GATTGAGGTCTGTCATAGGGAGAACAAACTAAAGAACTGGACTTTGCTTTCAGTCAAGATGGAGT
AACAGGG

| | |
|---|---|
| Sequence 1127 | cMhvSA033f06a3 |

CCCTTAGCGTGGTCGCGGCCGAGGTACCAGGTTCAAATAGTCAGCAGCTCATCATAATCAATGAGC
GAGGACATAAAGTAGGAAAAATGCATCACCATGGTGAGCAAGGAAGGCAAGTTATTGGAGGCAC
ATGTTAACACATAAAATATAAAATTAATATGATCACACTGGAAAGGCTTGCCTGAGCCCACAGTTT
GAATGCCTACAATAAGATGAGATGCACAACAAAAAGCAAGAGAACCTGATCAAGTGGGTGACCT
GGCCATGGTGCTCTCATCAGTGGGGACCCAAATGCTTATGTGGACTCACCAGGTATCGAATTAGAC
ATGAATAGGAGTGTTTGTTGTGATGGCAAGAAACTATATAATCAAATGAATACAATGAAACTTTA
AAAATAATTGTAAG

| | |
|---|---|
| Sequence 1128 | cMhvSA033f11a3 |

CCCTTTCGAGCGGCCGCCCGGGCAGGTACAGACGGTCAGAAGGAAAGAAGGAGAGGGATTGCCT
GCTGCCTCCCCGCGTGCACACACGAGAGTGGGTGCTCCCACCAGCTTTCAGGGGGCTTTCTTCACG
AATGTGAGCACTGATTTTGGGAGATCTGCAGTGGAAAGTCAAGTCATGAATATTTTTTATAAAGAG
AGAAATGATGTAATTTTATCACAGAAGATATTTCAGATGTATTTTTCCATTTTAAAAATTCATTGGC
AGTGCTCATACAAGAGAATTACTTGACTGAAAATGACTCTGTCCAGTTTCTTCCTATTTCGTTAATG
ATTTTGCAGTCACTGAATTCTTTCTAAAAGTTGTATAACCCAGATAAAGTCAGGCCTCCTGGAAGC
CAGCTTCAG

| | |
|---|---|
| Sequence 1129 | cMhvSA033h06a3 |

GGTACAGGAGGCAGCTTTTTTCTGCTCTCTGTTGACTTCTGAAGCCAGCCTCATGATCGTTTCTCTG
CTAGCTTTTGCTTCCATCTCATGGACATNTATAGTCTCTTCAANAATAACAATTTGTCCTTTCACGA
ATTCATTTTCTTTGCGCAGGTCTCTAAGCTGAAGAGAAAGCAATTACAGCTGTCCTATAAAAATTA
ACAATTNCATCATTTTCTCTAAGCAAGTCACATCTATAGACTGCATTATCATATGAAAAATGTAAG
AGCACTATCCCTACATGGACTGGAAAGGTCACATTTTCAAAGGCAGCCTGTAAACTCTGNGNTTAG
ACCTGGGGGNCAAATTCAAAT

| | |
|---|---|
| Sequence 1130 | cMhvSA037a05a3 |

CCCTTTCGAGCGGCCGCCCGGGCAGGTACTTTGCTACACGGCCGGGGGCCATTGAGACTGCCATGG
AAGACTTGAAAGGTCACGTAGCTGAGACTTCTGGAGAGACCATTCAAGGCTTCTGGCTCTTGACAA
AGATAGACCACTGGAACAATGAGAAGGAGAGAATTCTACTGGTCACAGACAAGACTCTCTTGATC
TGCAAATACGACTTCATCATGCTGAGTTGTGTGCAGCTGCAGCGGATTCCTCTGAGCGCTGTCTAT
CGCATCTGCCTGGGCAAGTTCACCTTCCCTGGGATGTCCCTGGACAAGAGACAAGGAGAAGGCCT
TAGGATCTACTGG

| | |
|---|---|
| Sequence 1131 | cMhvSA037a12a3 |

CCCTTTCGAGCGGCCGCCCGGGCAGGTACCTATTCTCTCAATTTTGAAACGGCAAAAAATTTTTAA
AAATTAAATAACATTCATGCTCTGTTTTGGACTGACATCCCAAGATTTTAGTGTAGGGCAGTAATT
TTCATTTTCAAATTACAATGCACCTTCCATTCCTCAGAGAAAAGTAAGTTTCTTTTTCTACCTCACT
GTCTCCTGGCTCTCAAACCCTCCTAGGCTAGTAAGCGTCTTCAGCCCAGATGAAGAAATAAGAAAA
TCCTATGGAAGGGCTTTCTTGCTTGAGGCTATAGTAACAGCCACAAAACACCCACACACTTTTAAA
ATTCTTACCTCGGGGGTAGGGATAGCATTAGGAGATATACCTAATGTAAATGATGAAGTTAATGG

| | |
|---|---|
| Sequence 1132 | cMhvSA037b03a3 |

CCCTTAGCGTGGTCGCGGCCGAGGTACCAAACTGCTGTCCCCAAATAAAGAACTTACATCAACAA
GGAATATAAAAATGTTATTTAGGACTTCTGTTCTCAGATGTTTAATACAAAGGAGAGATTGTTGTG
CCAGGGAACAAAGTGATCCAATATCCACGAAGCCAGAATTCTCCTACTGCACATTTTGTTTCCAAA
ACACTAAGGAATACAGCAAGATTTCAAGTTGGAGTAAAGAAGCTACTTCTGGAAACAAGAGAGGA
GATAACTGAGGACTTTCACAGAGGGGCTGAAATCCTTCCCGGAAAACTGTG

| | |
|---|---|
| Sequence 1133 | cMhvSA037d03a3 |

CCCTTTCGAGCGGCCGCCCGGGCAGGTACTACCGGACCTGTTTCATCTCTGCTTCCCAAGCCTCAG
GCCTGGGCCTCAGGGATTCTCTCCAGTGCATACCTTAGGCTACAGCTATAGGGCAGCTGTGGTTAG
GGAAGGTCCCTATTTAGAATAGTTGGTCAAAAAGCACATCACTTCTGTCCCTTTCTTGCAGAACTG
GTTGCTGCTCTGGAATGAAAGTTTGATTGGTCTGTTAGCCATGCCCACCTGGATTTGGGAAAGCCA
ATAGAAAGAATCTTCTGCTCTCCTATCTGCTGTTGCTTTTTAACCTGTAGCCTAAAAAATGGCATTA

| | |
|---|---|
| Sequence 1134 | cMhvSA037e10a3 |

GGACACACAGTTAACCACAAAACAGGCCTCTCTGAAAAAGCCATTGCCATGGACTGCCAGACAGA
CAATGACAAGACACAAATACCTTCTGGTGTGTGAGCCACGGGACATGTGAGCTTCCCCGCTGATGC
TCCTCTTATATCAAAGATCACTTTTACAAGATGAGCGACTCAATATCTTTTATCAAACCAATGATCA
CCTGCAAGCTATGGTATATTTTTGCAGCTGTGTAGAGCTATGTGGCATGAGAATGTGGGACTTATA
AATTGCTGATCCAATAAATAGACATTATGGGCAACAGTGTCTTATCAGCTAGTGTGTACCTGCCCG

| | |
|---|---|
| Sequence 1135 | cMhvSA037e12a3 |

CCCTTTCGAGCGGCCGCCCGGGCAGGTACTTTTCTTTATGAATGTTATACCAGAACTTAGGAGGAA
AAAATTTTTGAGCATACTGAATATTAGGAATTGGATATCTCCCTAAATTATTAAAGTTCATCTTCCA
TAAATTCTGTAAAACTGAATGTAGTATTTCCCCCTCTTCCCATGCAAGTAACTGATATCACTTTAGA
AAACCTGATATGAACATTATTTGTTATTGTGCTTTTATGAAGAATTCTGTCTAATCTTCTCATAAGA
AGAAAGAATTAGAACCAAAAATCTAATTATCAGATTTAGTAAGATGTAGGCAAGATCCCCTATTTT
TTTCATTTATGTCTTTCAAAATC

| | |
|---|---|
| Sequence 1136 | cMhvSA037g04a3 |

CCCTTAGCGTGGTCGCGGCCGAGGTACGCAACATGACATTGGCTGGTGTAAAGATCTTACAATTAT
TTTTAAAATTTCATTGTATTCATTTGATTATATAGTTTCTTGCCATCACAACAAACACTCCTATTCAT
GTCTAATTCGATACCTGGTGAGTCCACATAAGCATTTGGGTCCCCACTGATGAGAGCACCATGGCC
AGGTCACCCACTTGATCAGGTTCTCTTGCTTTTTGTTGTGCATCTCATCTTATTGTAGGCATTCAAA
CTGTGGGCTCAGGCAAGCCTTTCCAGTGTGATCATATTAATTTTATATTTTATGTGTTAACATGTGC
CTCCAATAACTTGCTTTCCTTGCTCACCATGGTGATGCATTTTTC

| | |
|---|---|
| Sequence 1137 | cMhvSA041a04a3 |

ACGGGGAGCCCCTTTTTCCTCTTCTCCAGGGTCTTAATAGGGTCTGGAAAGACTCACCTGGTCCAA
AAAGTTTGAGGAAGAAGCTTCTAGTCTTCAGCTCTGTAGGGTCAACATGAGATGCTTATTGTTCAA
GCCTGTGTGATCCACCCAAAAGTAGGCTGCTCTACTACGGCATCCATGCTGCTGTGACCGGATGGA
CCACAGGACAGTTGAGACCCCAGCTAGATATCTGCCAAACCCAGGACTGTCAGCAAGGGAATAGG
GTTCAGGTCTTCTCCATTTATAAACTACCAACCCCTCTTTACTCTGGAATATTCTCACTCTCCTGGCT
GGGATAGACAGTGTTGGCTCATTCCACTCCC

| | |
|---|---|
| Sequence 1138 | cMhvSA041a07a3 |

CCCTTTCGAGCGGCCGCCCGGGCAGGTACGCGGGGAAACAGGCTACTGCTATTAAGGATTGCACA
ACTTCTGGGCAAGGCAGAGGTGGGTTTGGCTTTTTAAAAATTTTTTCAGCCTGTCCTCATGGAACT
ACATATTCTTTTCTAAGAACTTTTCATCCTAACCTCCCTACTCACATCTTCTAAGTGTCTCTGCTCTG
GTGGGAATGTGATGGACAACACAGAGCCATCTCAGAAGCCTCTGTGGCCACCACCAGGCCGGCCA
GGGTGCAGGGGGCCACTCCCTGGGCAGCCATAGGGTTCTCAGCAAGGTGCATTCGTCGTCCCTGCT
GAGAATCTGATGGGGCAGCATTTTTTTTTAATTAAATGCAAGCTGAGTCATTTCAAC

| | |
|---|---|
| Sequence 1139 | cMhvSA041b03a3 |

NNCCCTTAGCGTGGTCGCGGCCGAGGTACAACGNTAGCAGCAATTCAAAAGGGCATCGGAGACAA
CTAATCATTTCATAATGAGCGAGGGGAGAAGCAATAAAAGCCGGGAGCCCAAGGACGGCATGAT
AATTTTGCAGAGTCTCAGCTCTCAACCAGACTCACGTTCATAAAATAAACAAATGTTTTTGGTAAT
GGAAAGCTAATGTATACATTATTTAAGGATAGTATTAAAACCAGACTAGATGGATCAAGTAATAC
AACAGTTACCTCATTAAGCATCCTTTCTTTGGGGATGTGAAAAAGTTATTCTTTTTTTTCTTCTTCTT
TTTTCCTTTTGAAATGGGGCTTTATTAATTAGAGATGTAATGGGAAATCTTATTTTTT

| | |
|---|---|
| Sequence 1140 | cMhvSA041b07a3 |

CCCTTTCGAGCGGCCGCCCGGGCAGGTACATGGCTAAAATCATTATACTTTCCCCGTCTTATGATA
ATCTCAGCAAAACACAAGCACGGATTCTTTCCTAGTCTTCCTGCCCATCCACCGCCCGCCATTTTCC
CTGGACCCCGTGTGATGACAGTGAGGCCTCCTTATTCCTTGTCCAGCAGGGATTGTGGTATGAGTG
TGTTCAGGGACAGTTATGAGTGGAAGTTGGGGAGAGACGTGGAAGGGCGGTTTTGTGTGGCGTCT
GTGCCATTACAGCCTCAGCTACAGAGACTGCACTTGCGGGCAGCTGCAGTGCTGGAAGCAGATGG
GGCCCTGTGCGAGGGGTCAGTGGAAGGCAGTGACTTTGAGAGCTCTGATGGTAGTTGT

| | |
|---|---|
| Sequence 1141 | cMhvSA041c04a3 |

CCCTTAGCGTGGTCGCGGCCGAGGTACTTCTATACAGTGGAATGCTACTCAGCAATGAAAAAGAA
AAAGATGCAACAACCTGGATAGACCTCAAAGGCATTATGTATAGTAAAAAGGTCAACCTTAAAAG
GTTATATATTATATGATTGCATTTATATAACATTCTCAAAATAAAAAAAACTATAGAGGATGAAGA
ATAGACTAGTGATTTCCAGGGCACAGGGACAGGGTAGGAAAGAATTGGTAGACAATGTGAATGCA
AAGAGGTCTCCTGTGTTGATGGAACAGTCTGTATCTTGATTGTGGTAGTGGCTACTCAAATCTATG
TATGGAATAAATTAAATAAAATTATACATATACACACAAATAACTGCAGGTTTAAAAT

| | |
|---|---|
| Sequence 1142 | cMhvSA041c06a3 |

CCCTTTCGAGCGGCCGCCCGGGCAGGTACAGTGGCCTAGATGGCTTTAGACTTCAGGATTCTTTAC
CATCTAGCCCCTTTTACTCTACCAACTTATTTTGTTACTTGTTGACATAATCTGTAGCCAGGAAAGC
CTGCATACAGTTTGTTATCCCTCTGTCTTTGCTCATGCGTTTTCTGCATCTGGAATCATCTTCCTCTC
TTCTCTCTGCTGGTTCATGTCCCTATTTTCTTTCAAAACTCTCTTTGAAATTTACATTTTTCAGGAAG
CCTTTCTCTTTGGCTTGCTGGACATCTGACCGGCATGTTATCTTTTCATATTTGTTCAAAATGTCATT
TTCAACATTTACTCAACTAATTAATATCAAGGACTTGCCATCAATTCTCTT

| | |
|---|---|
| Sequence 1143 | cMhvSA041c09a3 |

CCCTTAGCGTGGTCGCGGCCGAGGTACAGCTGTTGTCCATGTGTAGAGCTTTTAATAACCAGCGCA
GCAGGCCCCTTCACCTGCTTTTATGCCTGGACCAGATGACTGAATGTAGAACTTTAGGCACTTTTTT

| | |
|---|---|
| Sequence 1144 | cMhvSA041d09a3 |

GCCGCCCGGGCAGGTACACTGTTCTATATTTTAGCAGGGAAGGAATTTGTGTATGTGTGTGCTAAC
TANAAACAATGAGAAATAGCTCTAATGAAAGTTATATGGTCAGAATTTGGCTACAAGCTCTGCATC
ATTAGTAAAGCGGAGTATTATTGGCAGATGTCATGCTACTTTCCAAAAAGCCTGAACCCATCCTGA
TTTCTCCTTTCTTAGTTGAAATGCCAACAATTGCATATTTGCTTAATTATTGCTTTTTAAAATATTGG
CTCTGTATAAGCAAGGGAAAGTAATANAAAAAGTATTGTTCTTCCAAGTAAAGCAGAACACACCA
AGTGGACAATAGCAGCTTATATTTTCACTCAACATGGGATACTATTTTTA

| | |
|---|---|
| Sequence 1145 | cMhvSA041d11a3 |

CCCTTTCGAGCGGCCGCCCGGGCAGGTACACAGCATGCAGGCTGCAGCCTGGGCCCCTGCCAGGC
AAGATGTAGGGTGTGAGGTTGTGCTCTGCCCCATTCACTCTGGAACAGCTCCGCCCTTGAGTCCAG
GATATTTTCTCAGTGCCTCCACGCATTTGACCATCCAGAAAACATCCCAACTCAGTGTGCCTCGGC
CACCATAAATCAGCCAACCACACATGCTGCCCTCAATGCTTCTGAATATCAAGGGAAAGGATCTGC
CTCATCCTGCCCTGCTCCTGAGGCTTGCGCATTGACGCTTGAGTTATGTCATTATTTTTTTAAGTGA
TAGAAATCTAGTCAATGATTTGTAGCAATCACCACTGTGCAACGTATGCCAAAAAACTCTGT

| | |
|---|---|
| Sequence 1146 | cMhvSA041e02a3 |

CCCTTANCGTGGTCGCGGCCGAGGTACACCTCCCAATGTGGAGCCTGGAACCCTGGGAAGGGCAG
GCGGGCAGAGCCTCCTCACAGGGACTGGAGTCTTGGGAGGTTTACCCTATAGGAAGAGAGCAGTG
ATTCGTGTTGCTCAGGATTCCTTANATTCCTTTGGGAGAGTTAATCATCTTTACTACCCAGAGTGCA
CCCTTAGGTCTAGGTTGTCATACCCANTGATTGATATCTTANGGTAAAAGACGACCTGAGAATGGT
CTGGCCATGATCATAAAGATCGGATTGCTATGATCATGATCAGTCAGGGCTTTGGTGTTTTATTCTA
ATTG

| | |
|---|---|
| Sequence 1147 | cMhvSA041e05a3 |

CCCTTTCGAGCGGCCGCCCGGGCAGGTACGCGGGGACATTCAGAGGTGAGCCCAGAGCGGGTAAA
GTGGACTGGGGAGAACTTCGGAGGATGTTCATGTCCAGGAGCAGCCCCACGCCCTGTATGGTCGG
TGTCTAGAGCCTCACAGCAACTAAGACCAACCCAGCTCTCAGAAGAAGGAATGTCAAAATGTCAT
GTTCAATTTTACATTCAGTGCCTGGAATCTTTTCTTCACAATTGAAATGAAATGTGCTGAAGGAGG
TGAATCCATGCATTAATCTTCAGCTCACAAAGGAAATACTACATAAGAAGCAAGACCACAGACTC
AAGACGGACATAATTGGATTTTTTTTGCCATGGCCTGGAAAG

| | |
|---|---|
| Sequence 1148 | cMhvSA054a03a1 |

CCCTTTCGAGCGGCCGCCCGGGCAGGTACAAAAGCTGAGGGAAAAAGTTTCAGCTTCAAGCATTA
ACGTTTTAGTTCATAAATCTGAAGGAAAATAAAGAGAAAATAAAGGCATTAAGAGATATGAAACA
ATGTAAAAATGAATATTTCTTTTANGAATCCTTGTGAATATATGACAGTATACAAGCTACAGAAAA
CTAGTTTACTGGGAGGATCACGAGGTCAGGAGATCTAGACCATCCTGGCTAACACGGCGAAACCC
TCTTCTCTACTAAAAAATACAAAAAATTAGCCAGGAGTGGTGGCGGGC

| | |
|---|---|
| Sequence 1149 | cMhvSA054d12a1 |

NGGGGCCCTTTCGAGCGGCCGCCCGGGCAGGTACAAGGTTGGTAGGAGGAAGAGAAGAAATGAT
TGGCTCCCAGAGGCTTCATGGGCTCCCAATTCATGATTCTTTCTCTGTGGCTAATTTTTGTTAAGTA
TAAGAATTCCAGGAATCTCTTAGGAATNGGGGAGACTGCTTTCTCCTGAAATATAAAACATCTGCT
CTTGGTCTGTTTGGCGCTCCACTGTCTGAGGGGAAAACAGGGAAAAAGAGGTAATATAAAACAGA
CATTGTTTCANACAATAAATCCCCCTTTACTCATTAATGAGAAAATAAATTTAGG

| | |
|---|---|
| Sequence 1150 | cMhvSA054e02a1 |

NNGGGGCCCTTAGCGTGGTCGCGGCCGAGGTACACCAGGCAAAAGACAGTGGGAGCCCTACCTAA
GGTCAAGGCAGAGGGATAGAGAGTAGGAGACAGATTCTAGGTTACAAAGTTACAGCTGCAAAGA
CTGAGTCAGCTAGTTGTGGTCACGGGCAGGAGTAGGAGGAGGAAGGTAGGGGCTAGTCAAGGTCA
GCGTGTTGGGTCCTGCTGCGGTCACTGCCAGGTTCTTCCATGGCTCCGAAGGTGGACCACAGGAGC
TTTCTCCATCCCCAGAAAACCTGTTGTCAGCTCCTCTGAACTCCATCTACTGTGCATGTGGCAC

| | |
|---|---|
| Sequence 1151 | cMhvSA054f04a1 |

AGCGGCCGCCCGGGCAGGTACACACTAGCTGATAAGACACTGTTGCCCATAATGTCTATTTATTGG
ATCAGCAATTTATAAGTCCCACATTCTCATGCCACATAGCTCTACACAGCTGCAAAAATATACCAT
AGCTTGCAGGTGANNATTGGTTTGATAAAAGATATTGAGTCGCTCATCTTGTGAAAGTGATCTTTG
ATATAAGAGGAGCATCAGCGGGGAAGCTCACATGTCCCGTGGCTCACACACCAGAAGGTATTTGT
GTCTTGTCATTGTCTGTCTGGCAGTCCATGGCAATGGCTTT

| | |
|---|---|
| Sequence 1152 | cMhvSA057b02a1 |

CCCTTTCGAGCGGCCGCCCGGGCAGGTACATGACATTTTGCACTCAGTGGTATCCCTAGGACTTGT
TTGAATACATTGCTGTATTTATCTAAAAGGGCAAAGCTTTCATTAAAAATAATCTAGTGGCAATGT
TGCACAGCCCTAATTCTCTACTACATGAAAAGTTATATTTTCAGGCCCAGAGACACAGGATTACAG
GTCAGTGATAGGCAATGCATATTTGAAGTATACCAAAAGCACCAAATAATGTAGCTGAGTATCCA
GAAGGAACTGACATAAAATGCAGGGGTCTAATTACTAGAGTCATTGCCACAGAACCAGTCATCGA
TGACTAAATTATGCACCTGGTTTCCTGGGAAAATCTGCAGTTTGGGGAACATTTCACTACACTTCA
GAGCATTTTAAGTCTTTAAATCATTTAGCTTTTAAAATC

| | |
|---|---|
| Sequence 1153 | cMhvSA057c05a1 |

GCCGCCCGGGCAGGTACCCTAAACAAATATTAATACATAGACTCTGAGTGCATGCTGCTCACCTAT
AAATTCATGCTTGGGTAAAAGAACATGCTTTTACGATAGTCTGAGTCTTAAAGAGAAAGGCATCA
AGTGCAGGTCACCTGGCTTCCCTTCTGCCATAGACACCAGATAAATTCCAAAAAATGCAGGGGAT
GTGGGTCTAGAGCTTTCCTAACTTTGTAATTATCGCAACTGGTTCTGAAAGTTACTATATCCTCAGT
AAAGAATTCAAAGAGACTAAGTCTGCTTCTCCAGGTCTCCAACTCTGAGAACACTTGGAACTCTGA
TGTAGATCTCAACATACTGAAATCCAGTTTTCCTGTCTCTAGCCTTTGACTCAGAAGCACCAC

| | |
|---|---|
| Sequence 1154 | cMhvSA057c12a1 |

CCCTTAGCGTGGTCGCGGCCGAGGTACCTTCTAAATGCCAGGCTCATNTACGGCCATACCACCCTG
GACGTGCCCAATCTCGTCTACGTGCCAGGCTTGGGGGCATATGCAGATACATGGGACAGATCCCTT
GGAGGATACAGACAGATAAGCTCATGGTTCCTGCACAGGGTGGTGTGGGCTCTTACTGCTGAGCT
GAGACCTATGTGGTGACTGTGTTGGACTGAACCCCAGGGAAAGGTGTGGGGTCGGGTGTGATGGG
CACAAACAGAAAAGTGGCTGNTATGATTCACAAACTTATTGCATGTCATTGTACCTGCCCGGGCGG
NCGCTCAAGGG

| | |
|---|---|
| Sequence 1155 | cMhvSA057e10a1 |

CCCTTTCGAGCGGCCGCCCGGGCAGGTACTGGGGCACTCATTCTGCATGCTCCGAGAGATGCACTT
CCAGTTTCCGAAGAAGGGTCCTAGAATGCTTTTTTGCACCCGGCTTTTTACCCTATCATCATTCATT
TTCCTAGGCAGTTTTGTTGTTTCCTTTCTTCTGCAAAGCCGGGTAGATGTCTCTCACAGACAAGCTA
GAAATGCTGAGAGCTTCTGATACTCTGTTTCCTGTGCCTCTGTCTACTGTGCTAAAATAAATACTTC
TAACTTCCTTTTTGGAAACCATAGCAATTATTTCATTGCTTTGAAGACCTTCATACTCCTGGTCCCC
ACCCTGCAACATGGATTCCTGTGGCTGCTTTCTTCCAAATGCTACAGTGCTCAGTGTTGACTTTTTC
AAGATGACTCACATGTAACTTAATG

| | |
|---|---|
| Sequence 1156 | cMhvSA057e11a1 |

CCCTTAGCGTGGTCGCGGCCGAGGTACTTTTTTTTTTTTTTTTTTTTTTNGACACANAGTTTCACTCT
TGTTGCCCAGGCTAGANAGCCATGGTGCAATNTCAGCTCACCACAACCTNTGCCTCCTGGGTTCTA
GCCATTCTCCTGGGAGGCANAGGTTGCAGTGTGCCAAGATCACGCCATTGCAATCCACCCTGGGCG
ACAAGAGCAAAACTCCATNTCATNTNAGAAAAAAAGAAAAAAAAAANGAAAAGAAAAATANATG
AGCATCATAATCAAAAAGGCAGCCCTAAGAATAAATGAAAAGTTCACAGAAAAAAATAAAAATG
CAAATATCCCTTAAACACAGAAAAAGTTTCTAAGCTCATTCTTAAAAGGGAAAATGCAAATAATT
AACTAATTAA

| | |
|---|---|
| Sequence 1157 | cMhvSA057h08a1 |

CCCTTAGCGTGGTCGCGGCCGAGGTACTTTTCTTTATGAATGTTATACCAGAACTTAGGAGGAAAA
AATTTTTGAGCATACTGAATATTAGGAATTGGATATCTCCCTAAATTATTAAAGTTCATCTTCCATA
AATTCTGTAAAACTGAATGTAGTATTTCCCCCTCTTCCCATGCAAGTAACTGATATCACTTTAGAAA
ACCTGATATGAACATTATTTGTTATTGTGCTTTTATGAAGAATTCTGTCTAATCTTCTCATAAGAAG
AAAGAATTAGAACCAAAAATCTAATTATCAGATTTAGTAAGATGTAGGCAAGATCCACCTATTTTT
TCATTTATGTCTTTCAAAATCAATCACATTCTATTATTCACCGATCCACTAAACAGATGTAGAATTC
CTATTATGTAGCAGGCATTGTTCTGTTAAT

| | |
|---|---|
| Sequence 1158 | cMhvSA057h09a1 |

GGTACGCGGGGAGACACATTCAGAGGTGAGCCCAGAGGGGGTAAAGTGGACTGGGGAGAACTTC
NGAGGATGTTCATGTCCAGGAGCAGCCCCACGCCCTGTATGGTCGGTGTCTAGAGCCTCACAGCA
ACTAAGACCAACCCAGCTCTCAGAAGAAGGAATGTCAAAATGTCATGTTCAATTTTACATTCAGTG
CCTGGAATCTTTTCTTCACAATTGAAATGAAATGTGCTGAAGGAGGTGAATCCATGCATTAATCTT
CAGCTCACAAAGGAAATACTACATAAGAAGCAAGACCACAGACTCAAGACGGACATAATTGGATT
TTTTTTGCCATGGCCTGTAAAGAAAGGT

| | |
|---|---|
| Sequence 1159 | cMhvSA058c05a1 |

CCCTTAGCGTGGTCGCGGCCGAGGTACATGTGCACAACGTGCAGGTTTGTTACATATGTATACATG
TGCCGTGTTGGTGTGCTGCACCCATTAACTCATCATTTACATTAGGTATATCTCCTAATGCTATCCC
TACCCCCGAGGTAAGAATTTTAAAAGTGTGCGGGTGTTTTGTGGCTGTTACTATAGCCTCAANCAA
GAAAGCCCTTCCATAGGATTTTCTTATTTCTTCATCTGGGCTGAAGACGCTTACTAGCCTANGAGG
GTTTGAGAGCCAGGAGACAGTGAGGTANAAAAAGAAACTTACTTTTCTCTGAGGAATGGAAGGTG
CATTGTAATTTGAAAATGAAA

| | |
|---|---|
| Sequence 1160 | cMhvSA058d06a1 |

CCCTTTCGAGCGGCCGCCCGGGCAGGTACTTTTTTTTCTTTCTTTCTTTCTTTTTTTTTTTTTGTATTT
TTAGTAGAGACTAGGTTTTACCGTGTTAGCCAGGATGGTCTGGATTTCCTGACCTCGTGATCCGTCC
GCCTCGGCATCCCAAAGTGTTGGGATTACAGGCGTGAGCCACGGAGCCCGGCCATAGGCCTGTTTC
TTATTCTATATTCCTGTTAATGTAAACCTCCTGAGATTGGAAGACAATCANTTTTACAGGGTAAGA
ATTGTTTTAATTATGTGGCAGCTTTTNTNCAAACATGAAGAGAAACATTAGAAATACGTTTAATAA
AATTCTCTATTATNTTGTTTTCTTTCAGTTA

| | |
|---|---|
| Sequence 1161 | cMhvSA058e02a1 |

CCCTTTCGAGCGGCCGCCCGGGCAGGTACGCGGGGGACATTCAGAGGTGAGCCCAGAGGGGGTAA
AGTGGACTGGGGAGAACTTCGGAGGATGTTCATGTCCAGGAGCAGCCCCACGCCCTGTATGGTCG
GTGTCTAGAGCCTCACAGCAACTAAGACCAACCCAGCTCTCAGAAGAAGGAATGTCAAAATGTCA
TGTTCAATTTTACATTCAGTGCCTGGAATCTTTTCTTCACAATTGAAATGAAATGTGCTGAAGGAG
GTGAATCCATGCATTAATCTTCAGCTCACAAAGGAAATACTACATAAGAAGCAAGACCACAGACT
CAAGACGGACATAATTGGATTTTTTTTGCCATGGCCTGNAAAGAAA

| | |
|---|---|
| Sequence 1162 | cMhvSA058e11a1 |

CCCTTTCGAGCGGCCGCCCGGGCAGGTACTCTTCCTTCCAGAGGTTTCCCCATGCCCTCTTTTGGAC
TTGATGGGGGTCATTTGGGACAATAAGGCCTGATAACTCCTTGGACTTAGGAAGCGAGAGAGCAG
GAATCAAGAAAAGCTTTTGTGTTTTTTGGTTTGTGTAGAAAATATGATGGATTGAGATAAAATTTT
TCAAAATAGGCCCAATGAAGAAGAGCAGATTCAAGGAGTAAAGGATTATTTATGAGGATGGCCTG
TGCAAAAAGACACCCAGAGATTTCATGCTGTTGATTCACAGAAAGCCTGTTCCTCTTCACTCCGTA
GAGTCCTCAGAGTCTGGATCATCCCTTACAGAAGATCCTTGATAATATTTCTGATATACCTCCAAG
GTTCCGTTTGTCAA

| | |
|---|---|
| Sequence 1163 | cMhvSA059a08a1 |

ACCTGGCTTCTCTTGGCCAGATCGAAGGACTGTAATATGATTTAAGTTGTGAATATGCCTTAGTAT
GTGAGATGTCTTTTCATATGAGGGAGTTCTTAACCTACTTTAGCTTAATCACCAGATCCTTTTGTCT
TTTATGCTAACACATAAAAAACACAGGCTTGGTATTACAGCTTTTTGTCTTCTATGCATGAGCAGTT
TTGTTTTGTATCCCAGGGATCCCAGAANAACAGNTTTGCTTGGCCAGGGTACC

| | |
|---|---|
| Sequence 1164 | cMhvSA059b06a1 |

CCCTTAGCGTGGTCGCGGCCGAGGTACGCGGGGAATTGCTAATGGGAATGGGGTTTATTTTGAGGT
GATAGAAATATTGNTGAAATTAGAAATTGGCGGTGATTGCTAATGGGAATGGGGTTTATTTTGAGG
TGATAGAAATATTGATGAAATTAGAAATTGGCGGTGATTGCTAATGGGAATGGGGTTTATTTTGAG
GTGATAGAAATATTGATGAAATTAGAAATTGGCGGTGATTGCTAATGGGAATGGTGTTTATTTNGA
GGNGATNGAAATATTGATNAAATTAGAAATTG

| | |
|---|---|
| Sequence 1165 | cMhvSA059c12a1 |

CCCTTTCGAGCGGCCGCCCGGGCAGGTACAAAACAAAGACCCTTGCCTTCACTGCACTCATGTTCT
AGTTGTGCGTTGTGCGTGTCTTTATTTCTCAATAAGAGTTTCATGGCCCTACCACCTAAAAATGCCA
CAAAACAACAATCCCACAATCCCATTCAGAAAGTGAATGCATTTAACTTGAAACACGCAGTATAA
ATCTAAAGGAACAGGGTCAAATAAATGAAGCTGAGGCTGTGGCTCATACTTGTAATCCCACCACTT
TGGGAGGCCCAGGTAGGATGTTCACTTGAGGCCAANAGCTTGTTACCAGCCTGGGCAACAAGGTG
AGACCCCATCTCTATTAAAAACAAACAAACAAACAAACAAACAAACATGAGGCTGAGAAAAAAA
TGGCAAGGGATATCAAAAACT

| | |
|---|---|
| Sequence 1166 | cMhvSA059e10a1 |

GGTACCTGGCTGTGCTAGACAGGGGAAAGGAGATGCTTTCATTGCTGGCATTTTAATGGGGTCCAG
GACACTATGGGGAGGGGATTTAGGAAGAAGGCTAAGCCAGCAGTGGAAGACATTTGGAAGCTTG
GGGCANTGGAATTTGCCAACTGAAACAGGAAGTATTTGGATAAATTGAAGGTATGGGATGATGGG
GTATGCCTGGGTTGTAGGACATGGAAGACGTNAGTCTGGGGCCTGCTTAAGTTCATCCCTNAAAAT
GTCTTGCCTAGGGACCACTGTGATTTTNTAATAATATCCCTTAATTCTACTCTAGATGATATCTTTT
AAAGAACCTTTACTTTTTGAAAAAAGTAAA

| | |
|---|---|
| Sequence 1167 | cMhvSA062a03a1 |

CCCTTTCGAGCGGCCGCCCGGGCAGGTACAGTCAAATGCAGAAGGCATTGTATTAGCTTTTTGCTG
CGTGTAGTTGAAAAGGTTTGGAGGTTTGGAGGTCGTTTTCTGGCCGGAGAATACATAATTCTTGGG
AAAATGAGCTGGAAGATAATGAGAATCTACCTTATTTCTCTGCACAGGAAGATCAGTCTGCCTGCA
GTTAGCTAATCTCCCTGAACCTTGCTCACTACATCAGGAGACCATAAAGCAAAAGGGTAAATCAA
CAGTTCCTTTAAGACACTTTATCCAAAAGGATTCTCCTTTCTTGCCTGTAACTCTGACAAGGACAGT
GAGGGTGAACGCTCCAACTGTCACTGTTCAGGAAAAGGCCAGCTTATCCTGCAGCCTCAGCTTCCT
GGGCGGATGATCCC

| | |
|---|---|
| Sequence 1168 | cMhvSA062d06a1 |

CCCTTTCGAGCGGCCGCCCGGGCAGGTACAGATTAAATAGGTTAACCTTTATGTGGGTAAATTATA
TCAATAAAGCTGATGAAGAACTGGTAGATGACAAGTGTAATATAAAGGCAACCATAAATACAAAA
TACAGGAATAAGCAATTTACTTAGAAGATAAAAAAGAAGGCTTCTGGCCAGGCGCGGTGGCTCAC
ACCTGTAATCCCAGCACCTTGGGAGGCCAAGGCAGGCGAATCACAAGGTCAAGAGAGATCGAGAC
CATCCTGGCCAACATGGTGAAACCCCGTCTCTACTAAAAACACAAAAATTAGCTGGGCGTGGTGG
CGCACGCCTGTAGTCCCAGCTACTCTCGGGAGGCTGAGGCAGAAGAATTGCTTGAACCCGGGAGG
CGGGG

| | |
|---|---|
| Sequence 1169 | cMhvSA062e11a1 |

CCCTTAGCGTGGTCGCGGCCGAGGTACATGCCTGTAATCCCAGCTACTGGGGAGGCTGAGGCAGG
AGAATTGCTTGAACCTGGGAGGCAGAGGTTTTAGTGAGCTGAGATCCCGCCATTGCACTCCATCCA
GCCTAGGTGACAGAGCGAGCGAGACTCCATCTCAAAAAAGAGAAAGAAGAAGAAGAGAGCTCAA
CAATGCAGCCAGGGAAGATTTCCTGTAGGAGTCTTGAGACAGGAGAAAGAGAGATGGAAGAGAA
AGAAAGCGCATGCTGCCTCTTGAAAAAATGGANAGATCACCCCCGCGTACCTGCCCGGGCNGCCG
CTCGAAAGGG

| | |
|---|---|
| Sequence 1170 | cMhvSA062f03a1 |

CCCTTTCGAGCGGCCGCCCGGGCAGGTACAGTGGAGCCAAGATTAGATCCAGGGGACCTGGTTTC
CCAGCCCCATCACCTCAGTCCTATTGCATTACCCTCTGGAAATGCTCAGTCCAGTAAAGGAGAGAG
TGATGATGCAATGATGTGACTGCTTCCAGTGAAGAGTAAAAGTAATGAACTAGAAACGGGAGAAA
CAGATTGACACCCTTGAGTTGTCTTTCTGGTTAGGGCTTTTGGGTTTTTGTTCTGTAATACAGTCCA
ATGTGGTGGCCATTCAAGGGAGAAGGACCACTCATCAGCCCTCCTGCTCCCTCACCCCCATCTTAA
TTAAATAAGCCTCCTTAGGATCTCACACACCTGCATGTAACAAAACAGGTTTTAAAAATCTG

| | |
|---|---|
| Sequence 1171 | cMhvSA062g09a1 |

CCCTTTCGAGCGGCCGCCCGGGCAGGTACGTTGTCTTGTGGAAATTTTAGAGTTGCTTCCTTATTTA
GGGAAGATAATTTACTCAACTCCCTTTGAACACGTTTGCTAATTCCATTTAGGTTTTATTCCAGTAA
ACAATAGAATTGACCCTAGTTTTACTAATCATATTAAATTTTTATATCTTAATTATAATCCAGAGAG
TATCCGCTGGCTAACCTAATCTGAAAATTAACTAACTCGTGGAGGAATATTCAAGCATTCGGATAG
TTTTAAATTCAACTGTGCTAATACAAAAAAAAATTAGCTNGGCATTAAAAGGTTAGAGGAGGATA
TGTTTGTAAAACTAAATGGACCGATGAAAACCTGGACTTTATATCATAGAAGAACAGAGTGAAGG
TAAATTGCACTGCC

| | |
|---|---|
| Sequence 1172 | cMhvSA062h07a1 |

CCCTTTCGAGCGGCCGCCCGGGCAGGTACTTACTTTGATTCCTCTAGTGCAAGATTATAGTGGGGT
TATACCTGAGACTTCAATAAATGTTTGACTAACTAAACTAAAATAGCTTAGGGTAAGGACTACTTC
CCCAAACGCCCTTTTAAACATGTGAGAAAGGGAATCTCCCTGACATACTGGTATGGCCATTTGTAG
CAATATACTGAGAGTGACTTGGGTGATTTTCTGGGGCGATCAACCACATTCCATGAGCAGGTTAAC
TGTGGAAGACACCTGCCCTTGAGCATCGCGTTTGGGCCACATGCGTCAATGGGGAAATTTGTGTTT
CCATTCTGCTTCTTGTTTTGCCTTCACAACTTCAGGGATAGAAGCGTATTCCATTTTTA

| | |
|---|---|
| Sequence 1173 | cMhvSA062h09a1 |

CCCTTTCGAGCGGCCGCCCGGGCAGGTACTTTTCTTTATGAATGTTATACCAGAACTTAGGAGGAA
AAAATTTTTGAGCATACTGAATATTAGGAATTGGATATCTCCCTAAATTATTAAAGTTCATCTTCCA
TAAATTCTGTAAAACTGAATGTAGTATTTCCCCCTCTTCCCATGCAAGTAACTGATATCACTTTAGA
AAACCTGATATGAACATTATTTGTTATTGTGCTTTTATGAAGAATTCTGTCTAATCTTCTCATAAGA
AGAAAGAATTAGAACCAAAAATCTAATTATCAGATTTAGTAAGATGTAGGCAAGATCCACCTATT
TTTTTCATTTATGTCTTTCAAAATCAATCACATTCTATTATTCACCGATCCACTAAACAGATG

| | |
|---|---|
| Sequence 1174 | cMhvSA002g07a4 |

CCCTTAGCGTGGTCGCGGCCGAGGTACGCGGGGAATTGCTAATGGGAATGGGGTTTATTTTGAGGT
GATAGAAATATTGATGAAATTAGAAATTGGCGGTGATTGCTAATGGGAATGGGGTTTATTTTGAGG
TGATAGAAATATTGATGAAATTAGAAATTGGCGGTGATTGCTAATGGGAATGGGGTTTATTTTGAG
GTGATAGAAATATTGATGAAATTA

| | |
|---|---|
| Sequence 1175 | cMhvSA002g09a3 |

CCCTTGCACTGTGACAAGCTGCACGCTCTAGAGTCGACCCAGCAATCTCCCTGCTGCTCCGTCGTC
CGCCAGGACGTGAAGCATTCCCGGGCGACGTTTTCTACCTCCACTCTCGTCTGCTGGAGCGTGCTG
CACGTGTTAACGCCGAATACGTTGAAGCCTTCACCAAAGGTGAAGTGAAAGGGAAAACCGGTTCT
CTGACCGCACTGCCGATTATCGAAACTCAGGCGGGTGACGTTTCTGCGTTCGTTCCGACCAACGTA
ATCTCCATTACCGATGGTCAGATCTTCCTGGAAACCAACCTGTTCAACGCCGGTATTCGTCCTGCG
GTTAACCCGGGTATTTCCGTATCCCGTGTTGGTAGTGCAGCACAGACCAAGATCATGAAAAAACTG
TCCGGTGGTATCCGTACCGCTCTGGCACAGTATCGTGAACTGGCAGCGTTCTCTCAGTTTGCATCC

| | |
|---|---|
| Sequence 1176 | cMhvSA032g03a3 |

CCCTTCGAGCGGCCGCCCGGGCAGGTACCAGAGGAGGAGATGGACGATCAGAGCCATGCGCCTGT
TTCCTGCACCCCCTGCGCACTGGTTCTATGGCCACAAGGAGTCTTACCCAGTAAAAGAGTTTGAGG
TGTATCCTGAGCTGATGGAAAAATACCCATGTGCCGTTCCCTTGTGGGTTGGACCCTTTACGATGTT
CTTCAATATCCATGACCCAGACTATGTCAAGATTCTCCTGAAAAGACAAGATCCCAAAAGTGCTGT
TAGCCACAAAATCCCTGAATCCTGGGTTGGTCGAGGACTTGTGACCCTGGATGGTTCTAAATGGAA
AAAGCACCGCCAGATTGTGAAACCTGGCTTCAACATCAGCATTCTGAAAATATTCATCACCATGAT
GTCTAAGAGTGTTCGGATGATGCTGAACAAATGGGAGGAACACATTGCC

| | |
|---|---|
| Sequence 1177 | cMhvSA033d01a3 |

ACTGCAGCTGGTGGGTCACCAGGACGACCGTCTTCCCCCTGAGTGTCTTCTTAATGCACTCCTCAA
AAATGTGCTTCCCCACGTGGGCGTCCACAGTAGACAGGGGGTCGTCCAGCAGGTAGATCTGACGG
TCGGAATAGACGGCGCGGGCCAGGCTGATCCTCTGTTTCTGCCCCCCAGAGAGGTTGAGGCCCCGC
TCTCCAATCTCTGTCATGTCTCCAAAGGGCAGAAGTTCCAGGTCCCGATTCAGGGAGCAGCAGTGG
AGCACCTGGAGGTATCGGGCCTTGTCATACCCGCGTA

| | |
|---|---|
| Sequence 1178 | cMhvSA037e06a3 |

ACTGAACTGGGAGGTTTTTAGTCTGATAGCCACAATTTTGACCTAGGCAGGAAGCTTTACAGCTTG
AGGCAGTTTCATGGTCTGAAGACAAACTTCTTGTGACTTGCTGCCGGTGTTGGACTGCAGGAGAGA
GCCTCACTGGGTCAGGAGCACGAGAACAAAGTGGATCCCACTA

| | |
|---|---|
| Sequence 1179 | cMhvSA054c03a1 |

NGGGGGNCNTTAGCGTGGTCGCGGCCGAGGTACATTGGTATGAGGGTATTACTGGGACCAGGCAG
GCCAATTCGTGGGCACCCAGGTGGCCTGCTCAAATACTGGTAGTGGAATCAGTGGATTGAGCAGA
TGAGAGGGTTCTTGAGTCACTGGATAACCNGNGTGATGTGGGTGATGGTAGTAGTGGGATGATCC
TCTGGGGCCCAAGTGTTGCACACTGATGTTGACACTGGCTACAGTGCACGGTCACCAGCCAGAGTC
CCAGACACACAACTCTCAGGTTCTTCCACTCTCTGTGACAGGGG

| | |
|---|---|
| Sequence 1180 | cMhvSA002b03a3 |

ACTGAAAAATNTCATGTCCTGGGAAACCCCTCAGTCCTGGGCAAACTGAGACCGGTGGTTATCATA
CAAAGAGAAAACCAAATAAGACTAAAATTATGTCCAAACACTTTCATTGTGGCTAGGAACACAAG
TTGAACACCCTAATAAGGAACACAAATAATAAAAGCTTGCATTATTGAGTGCTTATATGGGGTAA
GTATTATACTATTATCTCCATTTTAAAGATAAGCAAACTGAGACATAGTAAGGGTAAATAAGTTAG
TTAGTGAAGGCACCAGAATTTAAACCCAGAAAGTTTGGTTTTAGAGCATACACTACAATCAGCACT
GTATGGAAAGATATNTAAGAGCAGAGACAGGCNGAGATGGGAGCACTGGGGAAGACATCATGGA
GGGGCTAGATGGCTACATCTTGGCTTTAAAAAGTGAGCAAAAGTAAAAGTTAGAAAGGAGATGAA
AGTATCATTTATAAATGG

| | |
|---|---|
| Sequence 1181 | cMhvSA002b03a4 |

CCCTTTCGAGCGGCCGCCCGGGCAGGTACTGAAAAATCTCATGTCCTGGGAAACCCCTCAGTCCTG
GGCAAACTGAGACCGGTGGTTATCATACAAAGAGAAAACCAAATAAGACTAAAATTATGTCCAAA
CACTTTCATTGTGGCTAGGAACACAAGTTGAACACCCTAATAAGGAACACAAATAATAAAAGCTT
GCATTATTGAGTGCTTATATGGGGTAAGTATTATACTATTATCTCCATTTTAAAGATAAGCAAACT
GAGACATAGTAAGGGTAAATAAGTTAGTTAGTGAAGGCCCAGAATTTAAACCCAGAAAGTTTGGT

| | |
|---|---|
| Sequence 1182 | cMhvSA002b04a3 |

ACAACCCTACCACTACTCTACATCATGGAAGTCTTAACGATTTAGGGTAATACGATAATGAGAATA
CCAATATGGATCTATTAAATGAGGAGCTGAGTAAGCTCCAAATTTCCCTCTAGATTGGTAAGTCTA
TAATTTATTATATGAAATTCCTAATTATTACCATACTAAGTTCAAAAGATTTTAACCCAAATCCTTT
AGTAACTGATAAACCTCATTCTTAAGATTCTTGACAGAAATAATCTTGATGAGCTTCTTCTCTTCAT
GATCTTTCCAATGCTGTTATAATTTTGAGGGAATTACTCTTATTTTCATTAATTCTGTTGCAAGGAG
GAAAAGACTGACTCTGTGTTGGGGTTTCTTTTCTCTATAAGGCACAAGACCTAAATGTCATTGAAG
AAGTGATTCGAATGATGTTAGAGATCATCAACTCCTGCCTGACAAATTCCCTTCACCACAACCCAA
ACTTGGTATACGCCCTGCTTTACAAACGCGATCTCTTTGAACAATTTCGAACTCATCCTTCATTTCA
GGATATAATGCAAAATATTGATCTNGGTGAGTGTNAATGAAGACATTTATTATGAATCTTTT

| | |
|---|---|
| Sequence 1183 | cMhvSA002d10a3 |

ACGCTAGGCCGCGGCCTTCTTTTCTCCCAGAAAGGTGACCCTCCCCACCCTGCGTCCTGCTCCTTCC
GTCCATACTGATGTTTGTTTTGCTGGAGGCCAGTAGCAACTGGACAGTAGCTCTAGGGGAGGAGA
ATCCACCTGCGGCGAAGGGTGGGATTTGTTTTCTTTGAGCCTTCTCCAGTGTGGGGCAGCTGGCGC
ATCTCCACTTAGCGCCGGGGGTCCGGGATCCTACATCGCAGGGACTGGGGATCTCCTGGGTTCTGT
ACC

| | |
|---|---|
| Sequence 1184 | cMhvSA002e06a3 |

CCCTTAGCGGCCGCCCGGGCAGGTACAGAGCTGGAGGCCCAAACAGCCAGCCAAATCTTGCTGTA
TTTTATCCACCATAGTATAATCCAGAGACTGTGGACCCCAAATTGGGATGCTTTTAAAATCCAAAG
TAGTTCTGTATACACATTTGAAGAAAAATGCTGTTGAAGAAATGTATCCATAAAACACTTCAGGTC
AAAAAGCAAAAGAATATCAAGAAAAAGTTTAAATAACATGATTCCTACTGGTTTTAGATCATAAT
TATCATCCTATATTATTTATATTCGTATCACTGTTATCTTTCTCTGACAAATAATTCTGAAATACAAT
ACATTTTAAAGTTATGCAGGATTTTAAAGACCTCGTCTTCAAGCAAATACAAGAAGTTTAATAACA
AACTTTAAATAAATGCTCATTTAAATAAAAGTTTATTTTTCTCCTGGCCAAATATTTGGTGAATTAC
TTACAAAGATACTTTCAATGATTAGATTCCTTAGCTTAAAAAAAAATTCATTTGAATACGCTTTAG
CCCAA

| | |
|---|---|
| Sequence 1185 | cMhvSA002f09a3 |

GGTACCTGAAGCCTCTGTCTGACTTTCCAGTTGGAAAGGACATGCTTTTGTTTCCCACCGACTGTTT
AATTTTTTTGGCTGCAATGCATTTCTTGCCAGACGGGGTCTGTTTATTTGGATCAAACTGAGAAGA
AACTTTGGATTTGCTGTTTCCAGCAAAAGCCTTGAAGTCTGACTGGCTGTAGTCGTAAGGCGTAAA
CTCTTTTTCTGGTGGCTCTGGGTCCTTTGGCTTCTTGGAAATTTTGAGTCGTTTCTTCTCTTGTTTCT
GTTCTGTGGTCCTTGGGTCGCTTGTTGCTCGCTCTCTCTTCTTTGCAGCATTTTCTAGCTGTAGATCA
GGAACAGATGTGGGGGAGGAACAGGGAGGCACATGGGAACAGGGAACTCCACCGGCCTCAGCAA
TAGCTGGGACCCAGCTGCCTAAGTGGTAAGAAGAACAGTCAGTGGTGGGGAGAGGAGCTGTGGCT
GGAACTTCGGGACCAACACTCANGGTCAGCTGAAACAAATTCCTCACTGGACAATGACATGANGT
CATTTAAGAAANGCAAGCCNGCCAGGTGCANTGGCTTCATGCCTATAATTCCAATGCCTTTGGGTG
GNCTAAGTNGGAAGACTGCTTTAAGCAATCTGAAACANCCCNGGCCAACATAACAAGANCCTATN
TTTCNAAAAAAAAAAAAAAA

| | |
|---|---|
| Sequence 1186 | cMhvSA002f12a3 |

CGAGGTACGCGGGAATTGAATGTCAACTTTAGCTGTGACTTTTCTGGCAGCTAGAATAAAAGTAAG
ATCGTTGTCTGATAGAACTGAATGTCTCAGTTTATTAGAACAACAAAATACTGTAATCTTTCTCAA
AACCTACATGGAACAAACTGGAACAAGTATTTCATGAAAACCAAATGAAAAATAAGTAAATAAAT
GATTTCATCACCACTGTCACCAAAAACAAATGAATTTTTTGGATAGGAAAACATGGCTAAGTTGGT
AATTGACTGAGACATTGGCCTGGTGTGTTATCTGTGGTTGTATTTTATTAAACTTATATTTACAGAA
ATGGAAAAAAACTAACTTTTCATACAGTTTGGTGTATTCATAGCAAAATATGAATAGAAATCACCT
CTGGAATCTTGATGAACAAGGCCTTTAGTGGTTCATTGGTGTAGAATGAATATCAATTTAGAGAAA
TAGGTCTATAAGTCAGGAAGTGATGCAGAAATGTCATAAGGCTTATTCATAATCACAACATTTTTC
AAATTTTTCCACGTTAAATCTGAAATTTTAATTTCTTTGATAAAAAATCTGGTATTTTTGATTTTTTT
TACTTTTGGTTTGATTTGGAAA

| | |
|---|---|
| Sequence 1187 | cMhvSA002f12a4 |

CCCTTAGCGTGGTCGCGGCCGAGGTACGCGGGAATTGAATGTCAACTTTAGCTGTGACTTTTCTGG
CAGCTAGAATAAAAGTAAGATCGTTGTCTGATAGAACTGAATGTCTCAGTTTATTAGAACAACAA
AATACTGTAATCTTTCTCAAAACCTACATGGAACAAACTGGAACAAGTATTTCATGAAAACCAAAT
GAAAAATAAGTAAATAAATGATTTCATCACCACTGTCACCAAAAACAAATGAATTTTTTGGATAG
GAAAACATGGCTAAGTTGGTAATTGACTGAGACATTGGCCTGGTGTGTTATCTGNGGNTGGATTTT
ATTAAACTTATATTTACAGAAATGGAAAAA

| | |
|---|---|
| Sequence 1188 | cMhvSA002g03a3 |

NGGAGTCGACCCCGCGTCCGCTTACATATAATGCAACTTATATGTAAGTTTCATCAACACAGACTG
AGTATATAAGTTGGCTAAAAGTAACAATACCCATCTAACAGTACAATGCTGTCAGAGACCCAGGC
TCTTTCTGGCTTATTGTAATTCATTTCCTTAGCATGTTGGGTTTTATCTTCATTCTGTTCCCTTCACA
GTTGTGGAATTCCTGTTGCAGCTTCATTTTTTAAGGACACAAGGCAGGAAAGGGGAAGGGCAACT
CCACACGTGTCTGTCTTCTTATCTTGAATTGCAAAGCTGTCCCAGTACCTTACCACCTACTTGCTTC
TCTAGCAGATTCTCTTCCATATTATTTAAGCCACTGGGTCACTCCAGGTTACAAAGGTAGCGGTAT
ATTGAAACTTTGAAATTTCAGCCTCCATAGTAAAGAAGGGCAAGGGAGAAACGGTGTTTGTTTAGT
CAGTCTAAATTGTCAAAGGAGATAGCCAGATATCTCTTTTTGAGAGATAAACAGACACTCTTCATT
TAAACATGGTATAACTTGGCTTTAAGGCATATTTCTTTAAAAATATATTGTCAAGGACTGCGAAGA
GCCTGAAGCTACTTTGCCATACTTTCANGGCTAGCAGAAGACAGGAGAATATTTGGTCGGGGAAA

| | |
|---|---|
| Sequence 1189 | cMhvSA002g06a3 |

GATNGTTTTTTGCANAATNNNCCCTTTTNGNGGGGGTGAGGGGCCGNNNGNACCTAAAANNCNTT
GTTTTAANACNATNTGNTGCNACNTTTTGNCAAANCCAAAGAAACGGCCCTTGTCGCCCACGACA
CGTTTGCGTAAGGCGCAAAGCTGGAAAAGTGCAAGTCCTGTGGCTTTCCAAAAGGCAGCGGGAGG
CATTGGTGCCGGTTTATTTTTAAG

| | |
|---|---|
| Sequence 1190 | cMhvSA003b08a3 |

NNAGCGGCNGCCCGGGCAGGTACCCATNATGCNCACTGCAGGCACAACTCCAGATGAAGGACTAT
NGAATATATGAATCGGCAACGANNATGGAGGTGGTCCTGGGGGTGATTATTGCAGCCATGGGGGC
NCTGCCCANCATCTGAGCCAAGGGTNTTGNAANGAGAATGGAGAAGCTTTTTTCAGGGGGCTCTT
GGGACNATCAGGGCCCCCCCATGNTCNCATNTATGTCCTCGCCTNAAAAAAAACTTTTACCGTTAA
GCTTTTAGNAGGGCTAACAAGACCTCCTTGCCCTTTTGAANTAAACNCCTTGAATNTACTTGGGCN
AATAACCAAAGGCCTTTTTCCCCCCAAGGGCTTAAATNGCCCCAGGAAGAAAACGGTTAAACCTT
CCCTTGGCTTCCCTTGGNNGGGGCAACCTTCGAGNGGGGNAGGCCATTTTTTA

| | |
|---|---|
| Sequence 1191 | cMhvSA003c02a3 |

CCCTTGCACTGTGACAAGCTGCACGCTCTAGAGTCGACCCAGCATGGATATGCTGCTGATGAAATC
ACTCACTGCATACGGCCTCAGGACATCAAGGAGCGCCGAGCAGTCATCATCCTCAGGAACTTGCG
ATGTTCTTCACCGAGGAAGCTTTCGCAGTAGATCTTATATGCGTCTTCCGTGCCTGACGGACGCGC
GGCGAACCAGCCGTTGTCAGTCATCACTTTCAGACCGCCAATAGAAGCACCGTTGCCCGGAGCAG
CAGTCAGGCGCGCGGTGATCGGGTCACCTGCCAGGGTGCTGGCGCTCACCATTTCCGGAGACAGC
TTANACAGCGCCGCTTTTTGTGCGGAAGTCGCAGCTGCCTGCAAACGGTTGTAGCTCGGCGCACCA
AAGCGTTTTGCCAGTTCGTTGTAGTGTTCCTGCGGGTTCTTACCGGTGACAGCGGTGATTTCCGCCG
CCAGCAGACACATGATGATGCCGTCTTTGTCGGGGGGACACGGCGTGCCGTCNAAACGCANGAAG
GAAGCCCCTGNC

| | |
|---|---|
| Sequence 1192 | cMhvSA003h02a3 |

CCCTTTCGAGCGGCCGCCCGGGCAGGTACCACTGGGCTTGCACTGTGTTCCAGGCGGTAGGGTCTT
CAACAGACACTCTGAGAGGTGGGATTGTAGGGCATCAGTTTCTGCAGACACACTACAAGTGTNTG
GCAACACTATTGNGGAGGCTAAAGTAACTCCATCTCANATGCTAATCCACAATGTTTGATTTCTGA
GTAACCCCAAGTTTTNGGAAGGCCNCNANGNNCNCNACCTTTNTCTNTNGGGGCCNCTGNAATAA
ANCANCCNTGTNGGCCAGGGNTTGTTNTTTTACAATTTGGTTNTTAAAAGGAAAAATACNTGGCTN
GGGGGCCNCCNGTTGGGCNTCATTGCCCCTGGTGGATCCCCAAGCCACCTTTTGNGGAAGGCCAA
NTGGGCAAGGGGNAGGGATCCAATNTTTGGAGGGTCACNGTAGGTTTNAAAGNACCCAGGCCCTG
GGGCCCAAACATTGGGGTGNAAAAACCCCNCAATTCCTTCTTNACCCNANAAAAANTTNACCAAA
AAAAAAAACCCACGCCTTGGGGGCCGTTTNGGTGGGCCGGGGGTTGGCCCCTTGGA

| | |
|---|---|
| Sequence 1193 | cMhvSA003h02a4 |

ACCACTGGGCTTGCACTGNGTTCCAGGCGGTAGGGTCTTCAACAGACACTCTGAGAGGTGGGATT
GTAGGGCATCAGTTTCTGCAGACACACTACAAGTGTCTGGCAACACTATTGTGGAGGCTAAAGTA
ACTCCATCTCAGATGCTAATCCACAATGTTGATTTCTGAGTAACCCCAGTTTTGGGAAGGCCTCCA
AGTTTTCTACTTTATCTATTGTTCCTTGTATAAGAGCATGTGGCAGGCTGTTCTTACATTGTTATAA
AAAAAATACAGCTGGGCGCGGTGGCTCATGCCTGTGATCCCAGCACTTTGGGAGGCANTGGAGGG
AGGATCATTTGAGGTCACNAGTTCAAGACCAGCCTGGCCAACATGGTGAAACCCCATCTCTCCAA
AAATACAA

| | |
|---|---|
| Sequence 1194 | cMhvSA003h12a3 |

ANAATTCNCCCTTAGCGGCCGCCCGGGCAGGTACAANACTTGGCCGAAATCTGTCAGGTCAGCCC
AACTTTCCTTGTCNGTGTCAAATGCTGTGCCTCTGTCCTATCACCGGGAGAAAAAAATGGGTTCAT
TGNGGACGCCCTGCCNAGTTTATTTGTTTNGTCTCGGGGTGGGGAATTTATACCCTTTTTGGGTNTC
CAAATCTTTNATATGAAAAANGGGNTCNCCCATTCNTTNCAACCGGACNTTTTCCTGNGGGCAATN
NTTAAAAAAANACNTAATNTAATGGTTCCTATTGNGCCTNTNCNATTGNATTGCCCTNGGGTCGCC
TTGGGGTATAATTCCCTNGNTGGCCAATTTNGGNGGACCTTGNTCCTTGGTGGANAGAACNTAATT
TTGGTTGGTGGCCAACCAATTATTTTNTTTNCCTANCTTAAAAANTTGGCCAANGAAAAGGAATTT
TAACCAAGGGGTTGGGGCCAAAATGGGGNACCAAAAGGTTTTTTCCTTCCTTCCTTGGCCCTGGCC
ATTCCCAAACCTTGGCCAAAATTCCTNAATTGGTNTTTNAACCAATTGGTNAATTTCCCCTTTTTTT
NACCTNACCTTAATTTTTTTTTTCCAAAAAAACCCANAAAGGNAATGGTTAATNGGGGCCTTTTNA
TTTTTTCNAAAAACCAATCCAATTTTTTTAACCTTTTTGGGGAATNNTTAATTGGGGCCGGGGG

| | |
|---|---|
| Sequence 1195 | cMhvSA009g03a2 |

GGTACAGCTGGGATTTGAACTTGGCATTCTAGCTCCAGCATCCATGGCCTTAACCACCATGCTGTC
CTTTCTCATTTTGATTGAATAGGCTAATACATTCCTTGTCCTTAGAATAGAGTCTTGCCTGTAGTAA
GTGTTCAGGTGGCAGCTTTAGGGCTCTCACTTATCCCATTGGACTGGGAGTCAGGCTTGATGCTTC
CACTAAGTATCACACAACCTTGGCAAGATTCTTGTGCCCCGGTGAAATGAAAGGGTTGGACTTGGG
GGCCTCAAGTCCAGCCCGCACTGCATCCTGATCTTCTCTCTCCATGCCCCATCACCTANACCCATCC
ACTGTGGAGGACAAGTGTGAGAAGGCCTGCCGCCCCGAGGAGGAGTGCCTTGCCCTCAACAGNAC
CTGGGGCTGTTTCTGCAGACAGGACCTCAATAGTTCTGATGTCCACAGTNTGCAGCCTCAGCTANA
CTGTGGGCCCAGGGGAGATCAAAGGTGAAGGTCGACAAATGTTTTGCTGGGGAGGCCTGGGG

| | |
|---|---|
| Sequence 1196 | cMhvSA009h07a2 |

CCCTTTCGAGCGGCCGCCCGGGCAGGTACTGTGTGACAATGACCTGGATATGGAAGCAGAAGGGA
GCTTCTAAGGACCGGAAGCTGAGAGTCTGTCTCCTGTCCCGGCCCGGACACTGGGGTTCAGGAAGT
TTAAGAACAGACACTGTCTTGACAGGAACCAGAGCCTCAGTGTCTGCAGGAGTTGCTGGCTGTTTC
CTGATGCAGTTGGAGCAGAATGGGATGTCCTGGGACAACAGAAATGTTTACCCATCTTGACTAGTG
TGGTCATCTGAAGAATGGCCTCCAAAGACATCCTGAGAACCTGGGAATGTTGCATGGATGAAGGA
ATTTGCAAAAGTGATTAAGTTAAGGAGCTTGAAATTTGTGGATCATGCTGGGTTACCCCAGTGAGC
TCTAAATGTAATCACATGTGTCTTTATGAAAGGGAGGCAGAGGGAGATTTGCAGACAGATGAGGA
GGAAGATGAGAAAACAATGGACACAAGAAAGAAAAGGTGATGCAGTTCANGGACCCAACCAATA
AAATGANGTGACCTCCAGATGCTTGGAGAAGGG

| | |
|---|---|
| Sequence 1197 | cMhvSA010c03a3 |

CCCTTAGCGTGGTCGCGGCCGAGGTACTTTGCTACACGGCCGGGGGCCATTGAGACTGCCATGGA
AGACTTGAAAGGTCACGTAGCTGAGACTTCTGGAGAGACCATTCAAGGCTTCTGGCTCTTGACAAA
GATAGACCACTGGAACAATGAGAAGGAGAGAATTCTACTGGTCACAGACAAGACTCTCTTGATCT
GCAAATACGACTTCATCATGCTGAGTTGTGTGCAGCTGCAGCGGATTCCTCTGAGCGCTGTCTATC
GCATCTGCCTGGGCAAGTTCACCTTCCCTGGGATGTCCCTGGACAAGAGACAAGGAGAAGGCCTT
AGGATCTACTGGGGGAGTCCGGAGGAGCAGTCTCTTCTGTCCCGCTGGAACCCATGGTCCACTGAA
GTTCCTTAT

| | |
|---|---|
| Sequence 1198 | cMhvSA018a03a3 |

CCCTTTCGAGCGGCCGCCCGGGCAGGTACGCGGGAGTTTTAATTTTTCCAAAGTATCATATGAATG
GAATCATGTGATATGTAGCCCATGAATCATGTATATGGGTTTTTCACTTAGTAGAGCACATTTAAG
ATTCATCATTGTTGCTATGTGAATCAATAGCTGGTTCCTTTTATCTCTCCGCAGCTCCTACTGCACT
GAGAAGCACGTGTTCTCCATTTCCCTGGGGGAGACCATTGTATTGGGCAGTTTGGAACAAAACACC
ATGGACTGGGAGGCTTACACAACAGAAATTTATTTCTTGCTGTTCTAGAGGCTGGGAAGCT

| | |
|---|---|
| Sequence 1199 | cMhvSA018b09a3 |

CCCTTAGCGTGGTCGCGGCCGAGGTACTTTGCTACACGGCCGGGGGCCATTGAGACTGCCATGGA
AGACTTGAAAGGTCACGTAGCTGAGACTTCTGGAGAGACCATTCAAGGCTTCTGGCTCTTGACAAA
GATAGACCACTGGAACAATGAGAAGGAGAGAATTCTACTGGTCACAGACAAGACTCTCTTGATCT
GCAAATACGACTTCATCATGCTGAGTTGTGTGCAGCTGCAGCGGATTCCTCTGAGCGCTGTCTATC
GCATCTGCCTGGGCAAGTTCACCTTCCCTGGGATGTCCCTGGACAAGAGACAAGGAGAAGGCCTT
AGGATCTACTGGGGGAGTCCGGAGGAGCAGTCTCTTCTGTCCCGCTGGAACCCATGGTCCACTGAA
GTTCCTTATGCTAC

| | |
|---|---|
| Sequence 1200 | cMhvSA018b12a3 |

CCCTTAGCGTGGTCGCGGCCGAGGTACGCGGGGGAGACACATTCAGAGGTGAGCCCAGAGCGGGT
AAAGTGGACTGGGGAGAACTTCGGAGGATGTTCATGTCCAGGAGCAGCCCCACGCCCTGTATGGT
CGGTGTCTAGAGCCTCACAGCAACTAAGACCAACCCAGCTCTCAGAAGAAGGAATGTCAAAATGT
CATGTTCAATTTTACATTCAGTGCCTGGAATCTTTTCTTCACAATTGAAATGAAATGTGCTGAAGGA
GGTGAATCCATGCATTAATCTTCAGCTCACAAAGGAAATCTACATAAGAAGCAAGGAACACGCAA
GAGATCTACAGCTCTGATCTCCANGATAGTGAAATGAGGTGGTGAATGATA

| | |
|---|---|
| Sequence 1201 | cMhvSA018e03a3 |

CAGGTACCCTTCACAATACATTGGCAAATTCTGAAGCTACAAAGCACAAGAGACCAGAAAGCCAA
GTAGAAAGCTATGAAAAACCATTTTTAGGAAGCTAGTATTAGAGTTCAAGACCCAGCAGTGAGGA
CAAGAGGCTTTTGGTGACTGTCTGGGGATTTCATTTGGAAAGTCTGGAGATTGGTGCCTTTTAAGA
AGGGACAAAACTAAGGGTAAGTGAACTTTGGTTCTAGGAATGGCAAGATCAGCAAGAAGATCACC
ATTGCCAACTGTAGCCTTTACACAATGTCATAGCAGCCCAAATTCAGTCAGCTATTGAATTAAGTT
TATTGTCTACTTGCCAAGCTAAAGAATGTATGAATGCTGTCTTTAGA

| | |
|---|---|
| Sequence 1202 | cMhvSA018f01a3 |

ACTTGCTTGGTCTCCCCTCCCTGGAAACGTTCTCAAATTGGTAAGAAAGGCAATTACAGGGCTCAG
CTCGTTTGTTTCCCACCTGTCAAAGCACTGTCCTTCATTGTCTGATGTCCAGTGTCTCAATACCATT
GTCTTCTTATTTATCTGGATTCTGGGGTTGTTTCAGGTGGGAGGGTAAATTTAGTCCCTGTTACTCC
ATCTTGACTGAAAGCAAAGTCCAGTTCTTTAGTTTGTTCTCCCTATGACAGACCTCAATCCCTCCGA
ATGATGGTTAGATGACTTCCTATTGGTTTTTCTATTGGCAAGATGGTGAACCATGGGTCACCTTCCT
CCATGACACCCTGACTGAGTTACCTCTAATATATGCAGAATTCTGCTGTATGTAGAAAAATTAAT

| | |
|---|---|
| Sequence 1203 | cMhvSA018h07a3 |

ACTGAAAAATCTCATGTCCTGGGAAACCCCTCAGTCCTGGGCAAACTGAGACCGGTGGTTATCATA
CAAAGAGAAAACCAAATAAGACTAAAATTATGTCCAAACACTTTCATTGTGGCTAGGAACACAAG
TTGAACACCCTAATAAGGAACACGAATAATAAAAGCTTGCATTATTGAGTGCTTATATGAGGTAA
GTATTATACTATTATCTCCATTTTAAAGATAAGCAAACTGAGACATAGTAAGGGTAAATAAGTTAG
TTAGTGAAGGCACCAGAATTTAAACCCAGAAAGTTTGGTTTTAGAGCATACACTACAATCAGCACT
GTATGGAAAGATATCTAAGAGCAGAGACAGGCAGAGATGGGAGCACTGGGGAAGACATCATGGA

| | |
|---|---|
| Sequence 1204 | cMhvSA031b01a4 |

CCCTTAGCGTGGTCGCGGCCGAGGTACCTGACATGGTGCCCAAGGACTGGGAGTAGAAGCAGAAT
CCCATCCACCTCCACCTAATCATACGGAGAAAGGAGACAGGAGCTGAGGGAGGGCAGTGCTATGT
CCAAGCTGTCAGCAAGCAGTAGGCAGAGCCCAGACCCCTGCTTTCCCATGCCCACCCCTCCCCAGT
TCAGGGCAAGGCCACCTCTCCAGGGCCTTTCCCTCCCCTAGAGAGGAAACTCCCCAAGTTCCTCTG
ACCAGACAGGAGAATGAACCAAGAGAAGAAAATTCCACTTAACACACACACCTGGAGCCTGAGG
CTGAAAGCTGGAATCCCAGACTTTGACACTCAAGAAGGCATCTCCACACTTTTTC

| | |
|---|---|
| Sequence 1205 | cMhvSA031b02a4 |

CCCTTTCGAGCGGCCGCCCGGGCAGGTACAGCTAACTGTGCTAGGCAGGGCAGCCCTGTGAGTTCT
ACTGCTGTCTTGGTTTTACAGAGGGGGAAGTGAGGCACAGAGAAGTTAATTAACCTCTGAAGTGTT
GCAGTCTAAGGCACAGAGGCACAGTTCCAGGCAAGGTTCATCTGAATCTTAAGTCCTCACTCTTTG
CCACCATCCTCCACTGCTGAGACCATCCCTGTGAGTCCTGCCGCTCTCCTCCCCTGGTCCATATTCA
CTGCTACTCAATGAGGCCAAGGAAGCCAATGGTCGTGTCCCCAAGAGGATATCTCTCCCCTCCTGA
GAATCTTTCTCATACATCTCAATTCTGAGATACAGATTGAGAAGCACCTCAGCAAATCCACTGCAT
GGAAGGCAAAACAACCTTGA

| | |
|---|---|
| Sequence 1206 | cMhvSA031c11a4 |

CCCTTTCGAGCGGCCGCCCGGGCAGGTACAGTCAGGGTTTTGTCATGTTGTTTAGGCTGGTTTTGA
ACCCCCGGACTCAAGCAATCCACCCACCTTGGCTTCCCAAAGTGCTGGGATTATAGGCATGAGCCA
CTGCACCCAGCCAATTCTCCAAATCTCACAGCCAAACTGCAACTAAATTCCATCTCAAACAAATAT
TCAAATGCAGAAGACTCACCCATCTAATCAAGGCAGTTTTAATATTTAGGGGAAAAAAAATGCCT
GGATAAAACTGTAAAACCAAGCATGATAGAAGAGATACTTTTAGGAATGGGGGAGGGATGACAA
AAATAAAACGAGAAGGTAGATAAGAATGGAAAGAATACTAGAAGACAGCCTGCCATGAGGTTAT
ATTTTACCAGGGGGGTGATGGGTGCACCCAAATC

| | |
|---|---|
| Sequence 1207 | cMhvSA031h04a4 |

CCCTTAGCGTGGTCGCGGCCGAGGTACAAGGACTACAGGTGTAATCCTCCGTGCCTGGCCTGATGT
TTTTTACATTAATAGAGCTTATAACTCATAAGAATTATGTTAGTCTGGTGTATATTCTGTTTCCTTCC
TGCTCCTGGAGAAAGACAATCATTTTGGCCTTGAATAATTTCTTAGAAATGCAGATGTAAAATTTA
AAATACACACACACACACACACACACACACACACNCTNTGTTCANCCAAAACACTAGCAAGCCTN
TAAAAGTNNGCCAACTGACATTTGNNNATATNCCTCACCACTCTATTGCAAANATGAAGAAACAN
GCTTATTGACATTTTANATGGCTAAACTAACTATGAGATNTAGGGCTTCTCTA

| | |
|---|---|
| Sequence 1208 | cMhvSA032b02a3 |

CCCTTTCGAGCGGCCGCCCGGGCAGGTACGCGGGAGTTTTAATTTTTCCAAAGTATCATATGAATG
GAATCATGTGATATGTAGCCCATGAATCATGTATATGGGTTTTTCACTTAGTAGAGCACATTTAAG
ATTCATCATTGTTACTATGTGAATCAATAGCTGGTTCCTTTTATCTCTCCGCAGCTCCTACTGCACT
GAGAAGCACGTGTTCTCCATTTCCCTGGGGGAGACCATTGTATTGGGCAGTTTGGAACAAAACACC
ATGGACTGGGAGGCTTACACAACAGAAATTTATTTCTTGCTGTTCTAGAGGCTGGGAAGCTCAAGG
TGCTGGCTGCATATTCATTCTGAGGCCTCTTCTGATGTGCAGGCAGCTGCCTTCTGACTTGTGCTCA
CATTGGAGAGAGGGAGTCAGCTTTGGTGTCTCTTCTTGTAAGGACACTAACCCCATTCACTAGGGC
CCCACCCTCATGACCTAATCACC

| | |
|---|---|
| Sequence 1209 | cMhvSA032c02a3 |

CCCTTTCGAGCGGCCGCCCGGGCAGGTACGCGGGGGTTCGAGGTTCGTTTACGCGCCGCTTCGCCG
TGCAGGTGGTGGCGAAGCGCTCCTCCGAAAGGTTTCGGAAGCTGGTGGTAGCTCTGAAGATAACG
CTGCGTTAGGGCATACTGCGGCGGAGGATGGAACTCCGATTGAAAGCAGTTGCTGGAGTGGAGCA
CGAATTTCAACAAGCCGCATGTTGAAGTGTGAGGCGTGAAAGGGTATGTCTGATATTTGCTTTAAA
ATGCTCCAGCAAAGAAATTAAGGGATGGATGAAGCAAAAGAGCCAGGTATGGTGGCTCATGCCTC
TAATCTCAGCACTTTGGGAGGCCGAAGCAGGCAGATCACCTGAGGTCAGGAGTTTGAGACCATCC
TGACCAACATGGTGAAACTCGTCTCTACTACAAACATAAAAGAATTAGCTG

| | |
|---|---|
| Sequence 1210 | cMhvSA033a01a3 |

ATTTGGGCGGTCAACGCGGGTGGAGAGGCCCATGTGGACGTTCACGGGATCCACTTCCGCAAGGA
CCCTTTGGAAGGCCGGGTGGGCCGAAAACTTAGGACTANTGNNCNTGAAACTNCCAAATCCTNCC
GTTCCAAACCCGTGAAGGGACCNAGATCCTGTANTCAAACTTGANNCGGTACCNCCGGNGGGGTT
CCCGGGCCGTTTANCATTCNNTCCCGTGCCGCACCCGNCCCGGGNGNNCCAAAATTTTGGCAATTT
CTTTCNCTTGAAAGTAAATNATTGAAGCTTTTTTCCAAACTTNCTTGANTGNAGNCCTTTGTGNATA
ACCCCNNNTAACCTTNGGGGGCGGGNTAANNCACNACTTAAAGGGGCGNGAANTTACNANACCC
CNCNTNNNNTTGGNCCCNTTNTCTTAATTTGTNNTTTNGGAAAAAACCNGAAATGTTGGAANTCCC
TTTGATTCNAAAAAAAAAAAA

| | |
|---|---|
| Sequence 1211 | cMhvSA033c02a3 |

GGTACACTGAGCCTAGAATATCTTGTGGGGTCAAAAGGTAAGGCAGTGCTCAAAAAACAACAGTA
AAATGGCAAAAATACATAGAACCCAACTTGAAGGGCATCCTAATGTAAAATCTAGAAAAATCTGA
GCACAAAATGTATTATAGTCATGGGTTATAACCAATATAATGAGAATCCAAGAGTCCAGACTGATT
TTTAAAAAATTGCATTTTTTCAATATAAAAGAAAATATCTTCCTTATAGTAACATTTTAATTGACAA
ATGTAGAAGTAATGATGGAAGTAGAAAATCACTGTTTGGCACACACTGTAGTAATAACTGTTTCAG
ACAAGAATTATCCACGAATGCTAAAATTAGTTTGGTGAAAGTATGATGAGAAACAAGATACTTAC
ATAGGTCCAAAGCATCTCCTGACAAGATACTTATTCATTCACAGAAAAAAAAATA

| | |
|---|---|
| Sequence 1212 | cMhvSA033c08a3 |

CCCTTTCGAGCGGCCGCCCGGGCAGGTACGCGGGGACTCCTCACCCAGCATCCATAAAAGCATGC
TGCACCTTTGGCACAGCGCGACTTCCCTGGCCCTCCCCCTGCGGACCAGTGAACCTCGCCCGAGGG
CTCAATAAAGAAGATTTTTGCCCTCTTTTTCTCACCTCTCAGCCTTATTGATCCATGGTGCCCTTCC
ATTGCCTTTCATTGGTGCCGAAACCCGGGAGGGGACACCTCCTAAGCCCCCCCAGAGGCTCAGGG
GGACTCCCCTCCTGGTCGGATCAGTCCTCTCCCTCAATCAGGTCANGCTTCTCCTCCACGGCCATCT
GTCCATTTCGTCCGGTTACTTGCTGCCAGGTCGCAGTTGCTGCAGCTACTCCAGTCCAATTCGGCCG
AC

| | |
|---|---|
| Sequence 1213 | cMhvSA033h10a3 |

CCCTTTCGAGCGGCCGCCCGGGCAGGTACTTTGCTACACGGCCGGGGGCCATTGAGACTGCCATGG
AAGACTTGAAAGGTCACGTAGCTGAGACTTCTGGAGAGACCATTCAAGGCTTCTGGCTCTTGACAA
AGATAGACCACTGGAACAATGAGAAGGAGAGAATTCTACTGGTCACAGACAAGACTCTCTTGATC
TGCAAATACGACTTCATCATGCTGAGTTGTGTGCAGCTGCAGCGGATTCCTCTGAGCGCTGTCTAT
CGCATCTGCCTGGGCAAGTTCACCTTCCCTGGGATGTCCCTGGACAAGAGACAAGGAGAAGGCCT
TAGGATCTACTGGGGGAGTCCGGAGGAGCAGTCTCTTCTGTCCCGCTGGAAC

| | |
|---|---|
| Sequence 1214 | cMhvSA033h11a3 |

CCCTTTCGAGCGGCGCCCGGGCAGGTACATCGGTCCCTTGACCATTACACCCACGGTGGCCCTAAT
TGGCCTCTCTGGTTTCCAGGCAGCGGGGGAGAGAGCCGGGAAGCACTGGGGCATTGCCATGCTGT
AAGTGGAAACATCTCCCCTCATCCCACCACTGCGGGGCAGCCTTTAGGAACATTCACAGACTTCAG
GAGATAATGTTTTTCAATAATAAGAATGGTCTGACAGTTTCAACTTTATTTGCTTCGTGCTGGGGA
ATAGTTGAAGGGTTTTTGACCCAGAGTTTGGGAAGTGACATATAGTTGACGTATTACAAAGACAG
ACTTAGCAGCAATATGAAGAGGGTGGATTGTAAGTTTTTAAGCTTTGGTAGTGGGGTAAGG

| | |
|---|---|
| Sequence 1215 | cMhvSA037c07a3 |

ACTGAAAAATCTCATGTCCTGGGAAACCCCTCAGTCCTGGGCAAACTGAGACCGGTGGTTATCATA
CAAAGAGAAAACCAAATAAGACTAAAATTATGTCCAAACACTTTCATTGTGGCTAGGAACACAAG
TTGAACACCCTAATAAGGAACACAAATAATAAAAGCTTGCATTATTGAGTGCTTATATGAGGTAA
GTATTATACTATTATCTCCATTTTAAAGATAAGCAAACTGAGACATAGTAAGGGTAAATAAGTTAG
TTAGTGAAGGCACCAGAATTTAAACCCAGAAAGTTTGGTTTTAGAGCATACACTACAA

| | |
|---|---|
| Sequence 1216 | cMhvSA037e03a3 |

ACTTTGCTACACGGCCGGGGGCCATTGAGACTGCCATGGAAGACTTGAAAGGTCACGTAGCTGAG
ACTTCTGGAGAGACCATTCAAGGCTTCTGGCTCTTGACAAAGATAGACCACTGGAACAATGAGAA
GGAGAGAATTCTACTGGTCACAGACAAGACTCTCTTGATCTGCAAATACGACTTCACCATGCTGAG
TTGTGTGCAGCTGCAGCGGATTCCTCTGAGCGCTGTCTATCGCATCTGCCTGGGCAAGTTCACCTTC
CCTGGGATGTCCCTGGACAAGAGACAAGGAGAAGGCCTTAGGATCTACTGGGGGAGTCCGGAGGA
GCAGTCTCTTCTGTCCCGCTGGAACC

| | |
|---|---|
| Sequence 1217 | cMhvSA041a12a3 |

CCCTTAGCGTGGTCGCGGCCGAGGTACTTTGCTACACGGCCGGGGGCCATTGAGACTGCCATGGA
AGACTTGAAAGGTCACGTAGCTGAGACTTCTGGAGAGACCATTCAAGGCTTCTGGCTCTTGACAAA
GATAGACCACTGGAACAATGAGAAGGAGAGAATTCTACTGGTCACAGACAAGACTCTCTTGATCT
GCAAATACGACTTCATCATGCTGAGTTGTGTGCAGCTGCAGCGGATTCCTCTGAGCGCTGTCTATC
GCATCTGCCTGGGCAAGTTCACCTTCCCTGGGATGTCCCTGGACAAGAGACAAGGAGAAGGCCTT
AGGATCTACTGGGGGAGTCCGGAGGAGCAGTCTCTTCTGTCCCGCTGGAACCCATGGTCCA

| | |
|---|---|
| Sequence 1218 | cMhvSA041b01a3 |

CCCTTTCGAGCGGCCGCCCGGGCAGGTACAGTGCCCTCATCGAAGCTCCTAAAACTTCCTGAAAAA
AATGAAGCTTTAACGTCCAGCTTCCACTGCTTAAACTGAGCACAGGACGTGCACTTGGATAGTAAA
CCAGGTGTCTCCTCAAAGCCCTAATATATTCAGCATCTCTATCAAAGGCGCCTTTCATTTGACTTCT
TTGTTCTGGCAAAGACTCTCTCCTTTTAAATTTTCTTTTTTTGTCCTTATTCATTGCAAAATATTGGG
CCAGTTTACCCCTATTGGGTTCATGCAGATGGATGTTTTGCAAATGTAATTTTGTGTCCTGGACTAA
AGACTGCAACCAGCCTCGGAGTAAACGAAAATGCCCACTGCGGATATCTGACACCTTCCATTCAC
AAGCATCTACAAATGAGTCGATTTCCAA

| | |
|---|---|
| Sequence 1219 | cMhvSA041f07a3 |

ACATGGGCACCTGGCTGTGGCTCATCTACTACCATATTCTTTGTTCTTCTAGATCCTTCTTGGCTTCC
ATCTTGGCAACTCCAAAGGCATGGTGGGGAAAACAGATGCAGAGATAGATGCCTATTTCTCCTGC
AGTCTCTTTCAGCATAGCAATTAGGCAAGTTATCAATAAGAGTATATAATCTATAACTTATAGTCC
ACATAAGGCTTCACTCAATTTGAAAAATTGCCAGTTCTGTCAAATATGCTAACACTCCAATAAGGT
ATTTATGACACAGAATCTTTATTTTTCCATCAGTATGTGCTGAAGCTACAGATGTTGAAACACGAA
CTAATCTTGTGGCTGATAAATGAAT

| | |
|---|---|
| Sequence 1220 | cMhvSA041f08a3 |

ACTTTGCTACACGGCCGGGGGCCATTGAGACTGCCATGGAAGACTTGAAAGGTCACGTAGCTGAG
ACTTCTGGAGAGACCATTCAAGGCTTCTGGCTCTTGACAAAGATAGACCACTGGAACAATGAGAA
GGAGAGAATTCTACTGGTCACAGACAAGACTCTCTTGATCTGCAAATACGACTTCATCATGCTGAG
TTGTGTGCAGCTGCAGCGGATTCCTCTGAGCGCTGTCTATCGCATCTGCCTGGGCAAGTTCACCTTC
CCTGGGATGTCCCTGGACAAGAGACGAGGAGAAGGCCTTAGGATCTACTTGGGGAGTCCGGAGGA
GCAGTCTCTTCTGTCCCGCTGGAACCCA

| | |
|---|---|
| Sequence 1221 | cMhvSA041h10a3 |

GGTACAGGTCATGGTGAGCAGGTGTTCTGAGGGAAGACAAAGGAAAAGCAGAGGGAGTGTTGAC
AATTCTGAGCTTCCATATGGCAGACATTCGGGGCCTGTTGGCATGGTCCTCAGAGCAGCAACAACA
GCATCAATTGAGGTTCATTAAAATGCAGAATCGCAGGTTCATGTGGACCTACTGAATCAGAACCTG
CATTCTAACAACAGTTTTCAGTGGTTCTTCCGCACATTAAAGTTTGAAAAGCACTGGTCTGGAGGA
GGAGGCTCTACAAAAGGGTTGGGTATTGAGGAGCCGAAAAGACAACCTGGAACTGAGATTCCCAG
GGATGACCTGAAAACAAGCATTTCAAAAGCTCAGAAA

| | |
|---|---|
| Sequence 1222 | cMhvSA049h12a1 |

CCCTTAGCGGCCGCCCGGGCAGGTACAGTATCCTATATTATTCCTATTTTAAGATTTAAAGAAAAC
CCTGAGGTTTAGATAAGCAAATTGCTCAAAGTCACGCAATGCCATAGTAGTGTTGGAGCTATGATT
TTCCAGAATCTAAGCTCTTAGTCCTGGGAAGTGCCTAGTGCCCAAAGAAGAAGACTGGAATAAAA
TAAGGCTGAATGGTGTGTAAGAACCAAATAACAAAAGCCTTGCAGACAATTTTAAAGGCTGTGAA
TATTAGTCTAAGAACAATAACAAGCAAAAAAAAAAAAAAAAAAAGTTTTAACTGGAGATAGTAA
CATGTGTTTTCTTTTCTCTTCTTTTCTTT

| | |
|---|---|
| Sequence 1223 | cMhvSA054f03a1 |

NTGTNATGGATATCTNCAGANGGGGCCCTTANCNTGATCCCNNCCCANGTACACNGCAGGTATCT
GGCTCCACCACACTNANGAACCNGNAGGAGGCANGGAGTGGATANTGTGTCAAGGATGACTGAN
CCCTNCTTCTGTGTAAAACAAGTTACACCTANATTCANAATANATGCTGNNGCAACATAAAATTAT
AAAAATTCACTGTAATTCACATCTTGGTGCCTGGGCACCANTTTTTAAATGT

| | |
|---|---|
| Sequence 1224 | cMhvSA054f08a1 |

CGGCNTTTGGGCCCAACCAGCCCGCTCGAGCGGCCGCCAGNGNGATGGTTTTTGCAGAGGGGNAA
ACNNCGCNCCCCCGGCCNANGTACNTAGAGCCTGAGTTGCTCCACAGGAATCCAGGAACTGNGCA
CANGAAAAGGANCTCAGCTGGTGGNGTGGGAAGATGGAAACCAACTTCTCC

| | |
|---|---|
| Sequence 1225 | cMhvSA057a05a1 |

CCCTTTCGAGCGGCCGCCCGGGCAGGTACAAATATTTTAAATATGGAAATCCTAATGCAGGGGGT
GGGCTGAGAGAGATTTTATAGAATATATGTATGTATGTCCAAAACAGAAGATACGGAATAAAAAG
CATGAAAGAAAGAAGAGGTTCCATAGCAAGGTATCAGCAGTTCCTCAGGGATGAGGATGGCGGA
GGCATCAAGGAATCTCAAGATGCTACCAAAATAGGAGCGGAAACATGGAAAGATGGAAGCACAT
GTATAATTCAAGTCTGTTCAGCAACTTGTGTGCCTCCAGCCTAAAAGTAAACCACAGTCATGTTCT
AAAGGTTCCGATTCATACACATGTCTGCTTGTTCTTCAGTTTTGGTTTTGCTACTGGGCTTTGATTCT
TTAATCCCCACCTGCTGAATGA

| | |
|---|---|
| Sequence 1226 | cMhvSA057a12a1 |

CCCTTAGCGTGGTCGCGGCCGAGGTACGCGGGAATTGAATGTCAACTTTAGCTGTGACTTTTCTGG
CAGCTAGAATAAAAGTAAGATCGTTGTCTGATAGAACTGAATGTCTCAGTTTATTAGAACAACAA
AATACTGTAATCTTTCTCAAAACCTACATGGAACAAACTGGAACAAGTATTTCATGAAAACCAAAT
GAAAAATAAGTAAATAAATGATTTCATCACCACTGTCACCAAAAACAAATGAATTTTTTGGATAG
GAAAACATGGCTAAGTTGGTAATTGACTGAGACATTGGCCTGGTGTGTTATCTGTGGTTGTATTTT
ATTAAACTTATATTTACAGAAATGGAAAAAAACTAACTTTTCATACAGNTTGGTGTATTCATAGCA
AAATATGAATAGAAATCACCTCTGGAATCTTGATGA

| | |
|---|---|
| Sequence 1227 | cMhvSA057f05a1 |

CCCTTTCGAGCGGCCGCCCGGGCAGGTACTGAAAAATCTCATGTCCTGGGAAACCCCTCAGTCCTG
GGCAAACTGAGACCGGTGGTTATCATACAAAGAGAAAACCAAATAAGACTAAAATTATGTCCAAA
CACTTTCATTGTGGCTAGGAACACAAGTTGAACACCCTAATAAGGAACACGAATAATAAAAGCTT
GCATTATTGAGTGCTTATATGAGGTAAGTATTATACTATTATCTCCATTTTAAAGATAAGCAAACT
GAGACATAGTAAGGGTAAATAAGTTAGTTAGTGAAGGCACCAGAATTTAAACCCAGAAAGTTTGG
TTTTAGAGCATACACTACAATCAGCACTGTATGGAAAGATATCTAAGAGCAGAGACAGGCAGAGA
TGGGAGCA

| | |
|---|---|
| Sequence 1228 | cMhvSA057g09a1 |

CCCTTAGCGTGGTCGCGGCCGAGGTACTTTACTCACCCTTCCTCTGACAGAAAAGGATGAAGTCAA
GGGCCTGGTAGAGGCACCACTAAGAAAGGCATCTGAAAGGACCAAAGAGAGTGACCAGCAAGCA
TTTTTTGCAAGGCTGAGGAGCTGACAGCTTCCATGAAAGGCTGGACCACCCAGTGGTGAAAAGCA
TCATCTGGGTTACCTTGTGCTGCCATAAAACACACCACAGACTTGGTGACTTAAACCACAGATATT
TATCTTCTCACAATCCTGGAGGCTGGAAGTCTGCAATCACGGTGCCAGCATGGTCAGGTTCTGGTG
AGGGCCTCTTTCCTTCTCACTGTGTGCTCTTTCTTGTGCATGGAGAGAGAGAGCATGAACAAGCCC
TCTACTGTCCCTCTTAGAAGGGCACTAATCCCATAATAA

| | |
|---|---|
| Sequence 1229 | cMhvSA058d09a1 |

CCCTTTCGAGCGGCCGCCCGGGCAGGTACTTTGCTACACGGCCGGGGGCCATTGAGACTGCCATGG
AAGACTTGAAAGGTCACGTAGCTGAGACTTCTGGAGAGACCATTCAAGGCTTCTGGCNCTTGACA
AAGATAGACCACTGGAACAATGAGAAGGAGAGAATTCTACTGGTCACAGACAAGACTCTCTTGAT
CTGCAAATACGACTTCATCATGCTGAGTTGTGTGCAGCTGCAGCGGATTCCTCTGAGCGCTGTCTA
TCGNATCTGCTGGGCAAAGTTCACCTTCCCTGGGATGTCCCTGGACAAGAGACAAGGAGAAGGCC
TTAGGATCTACTTGGGGAGTCCGGAGGAGCAGTCTCTTCTGTCCCGCTGGAACCCATGGTCCACTG
AAGTTCCTTATG

| | |
|---|---|
| Sequence 1230 | cMhvSA058g06a1 |

CCCTTTCGAGCGGCCGCCCGGGCAGGTACTTTGCTACACGGCCGGGGGCCATTGAGACTGCCATGG
AAGACTTGAAAGGTCACGTAGCTGAGACTTCTGGAGAGACCATTCAAGGCTTCTGGCTCTTGACAA
AGATAGACCACTGGAACAATGAGAAGGAGAGAATTCTACTGGTCACAGACAAGACTCTCTTGATC
TGCAAATACGACTTCATCATGCTGAGTTGTGTGCAGCTGCAGCGGATTCCTCTGAGCGCTGTCTAT
CGCATCTGTCTGGGCAAGTTCACCTTCCCTGGGATGTCCCTGGACAAGAGACAAGGAGAAGGCCTT
AGGATCTACTTGGGGAGTCCGGAGGAGCAGTCTCTTCTGTCCCGCTGGAACCCATGGTCCACTGAA
GTTCCTTA

| | |
|---|---|
| Sequence 1231 | cMhvSA059f04a1 |

CCCTTAGCGTGGTCGCGGCCGAGGTACGCGGGGGCAGTTCTTGAGTTCCACATGCAGAGCAGATG
CGACAGCTAGAAGTGAGTGGGGCCCAGACCCTGGCCCAGGAAGATCCACTAAAGGAGGCCATCCT
TCCGCCTTCTTCTGCAGGAGTCAGGATGGAAAGGCAGATGTAAAGTCCCTCATGGCGAAATATAA
CACGGGGGGCAACCCGACAGAGGATGTCTCAGTCAATAGCCGACCCTTCAGAGTCACAGGGCCAA
ACTCATCTTCAGGAATACAAGCAAGAAAGAACTTATTCAACAACCAAGGAAATGCCAGCCCTCCT
GCAGGACCCAGCAATGTAC

| | |
|---|---|
| Sequence 1232 | cMhvSA062f11a1 |

CCCTTAGCGTGGTCGCGGCCGAGGTACTGTTGCAGTGAGCTCAAGTGTTGGGTGTATCAGCTCAAA
ACACCATGTGATGCCAATCATCTCCACAGGAGCAATTTGTTTACCTTTTTTTCTGATGCTTTACTAA
CTTCATCTTTTAGATTTAAATCATTAGTAGATCCTAGAGGAGCCAGTTTCAGAAAATATAGATTCT
AGTTCAGCACCACCCGTAGTTGTGCATTGAAATAATTATCATTATGATTATGTATCAGAGCTTCTG
GTTTTCTCATTCTTTATTCATTTATTCAACAACCACGTGACAAACACTGGAATTACAGGATGAAGAT
GAGATAATCCGCTCCTTGGCAGTGTTATACTATTATATAACCTGAAAAAACAAACAGGTAATTTTC
ACACAAAGTAATA

| | |
|---|---|
| Sequence 1233 | cMhvSA057c03a1 |

CCCTTTCGAGCGGCCGCCCGGGCAGGTACCATGTGCCTGAGATGGAGGTGTTTGTGGTTGGGCAGG
CTGGCTTTGCTAATTTTAAATCCACCAAAATATATCATTTTGGCATTGACAGGTGTATTAGTCTGTT
CTCAGGCTCCTATAAGGACATACCTGAGACTGGGTGATTTATAAAGAAAAGAGGTTTAACTGACTC
ACAGTTCCGCATGGCTGGGGAGGCCTCAGCAAATTTACAATCATGGTGGAAGGGGAAGCAAACAC
ATCCTTCTTCACATGATGGCAGCAAAAGGAAGTGCTGAGAAAAAGGGGAAAAGCCCCTTAGAAAA
CCATCAGATCCCATGAGAACTCACTATGATGAGAACAGCATGGAGGTAACCACCCATGATTCCATT
ACCTGCCACCGGGTGCGTCCCACAACATGT

| | |
|---|---|
| Sequence 1234 | cMhvSA009d11a2 |

GGTACTGGGTGGGTGAGTGGGCTCAAGGCCTCCTGAGTAGCCTGGGTGGCGTGGGCAATGATGGT
AACAGAGGCAATGCAAAGCTTGTCTCCTTCTTGAGCTCTGTGCTCTTGAGTCGGCAGATGTTGTAA
GGGACTGTGTAGATCAACCTTTAGGACAGGAGGTAGCACCTAAAAGTGAGAACCAGCTGTGGTGG
TGGCAATAGAGTTTATGCTTGACCTTTGTTAATCGGGAGAAGTTCTTGGGCATTTCAGATGATGGG
TAGGGCCATGGAACTCTCAGTAGTCCTGGTCCCATGATCTGCCTCTGAAACAGGAGGGGTGGGAT
GTGGTAGTGGGATCCACTTTGTTCTCGTGCT

| | |
|---|---|
| Sequence 1235 | cMhvSA041g12a3 |

CCCTTTCGAGCGGCCGCCCGGGCAGGTACGCGGGGACACTTTGCTGCCGAAACGAAGCCAGACAA
CAGATTTCCATCAGCAGGATGTGGGGGCTCAAGGTTCTGCTGCTACCTGTGGTGAGCTTTGCTCTG
TACCTCGGCCGCGACCACGCTAAGGG

| | |
|---|---|
| Sequence 1236 | cMhvSA003e12a3 |

CCCTTTCGAGCGGCCGCCCGGGCAGGTACACCTTGTTGGGAGAGATGGGGGCAGCCCAAGAAAGC
TCCTCAGCGGACTGAAGAGGGAGTAAGATGGGCTGAGGGGAGCTTGCAGTTCATGCTGCATTAGG
AAGAGGGAAGCTCTTCAGTCCAAGTGCNGCCTGCAGGGGTGGGAAAAGCAACCAACACCGGACA
CCCGTTCCCACCCTTNAACCCCCCCACTGGGCACAGGGGTCNCCACCAAATTCTGGGGTCAAAANG
AAAATTAGGGCGGGGGGGCCCCCTTTGTGGGGTCCATTCCAAAAAGNCGGATNCCCAATGGGTTC
TTTTGGAGGGGCTTGGAGGGGANTTCANTGTTGCCAAGGGCCCCATTTAGNGGNTGGAAAAAAAT
TGGAAANGAAGNCANTTGNAAACCNAGNGGGNAGGGGTGGAAGNCAAGCCCCCCCCATTCCCAA
NGATTGNCCCCGGGGGGGGGANNTAAAAGGAAAGGCTTGNGGCCANCCAAGTTCNGGCCTTGGG
CCGGTTANGGGGAAAAAAAACTTGGCCTTCCCCCCCCATTTTACCCGNTTTGAAAAAGGCCCTTGG
GGATTCTTGGGGAAAGTNTCCCTTGGAAAGCCCATNNCANTTTTTGCCNCANGGGGAAAGAAGGG
GGCCTTGCCGTTTGNCCGGGGCCCCACNNAGGGGAAGNACTTANCCCCTTTTC

| | |
|---|---|
| Sequence 1237 | cMhvSA002c10a3 |

CCTTAGCGTGGTCGCGGCCCGAGGTACTGATAGTCTGTCTCGTTTACGAAGCCCATCTGTTTTGGA
AGTTAGAGAAAAGGGCTATGAACGATTAAAAGAAGAACTCNCAAAAGCTCAGAGGGAACTGAAG
TTAAAAGATGAAGAATGTGAGAGGCTTTCAAAAGTGCGAGATCAACTTGGACAGGAATTGGAAGA
ACTCACAGCTAGTCTATTTGAGGAAGCTCATAAAATGGTGAGAGAAGCAAATATCAAGCAGGCAA
CAGCAGAAAAACAGCTAAAAGAAGCACAAGGAAAAATTGATGTACCTGCCCGGGCGGCCGCTCG
AAAGGG

| | |
|---|---|
| Sequence 1238 | cMhvSA054a12a1 |

NGGGCCCTTAGCGTGGTCGCGGCCGAGGTACTTTTTTTTTTTTTTTTTTTTTTTTTTTTTTTTCTTTTT
TTTTTTTTTTTTTTTTTTCNANCCAACAATGTNTTTTNTTATGTNTNCGGGTTTNAAAATTNTTTNTT
NAATNTCTCCATNCCCAGNCAAAGGGANGNGTGTTNCTTAACATACTGNAAATTGCCTAACTTAAT
CATTNCCTAAAAAAAAAAAAATTN

| | |
|---|---|
| Sequence 1239 | cMhvSA054e05a1 |

NGGGGCCCTTAGCGTGGTCGCGGCCGAGGTACTAGGATTACAGGCGTGAAGCAGCATGCCACGCC
TATAGTGATATCTTTAAGTAAGCCTCTCCTATCTTTTTTGAGCAGTTTTTCAAAGCAACAGGCACCT
TATTAAATTAGAAAGTTGATGTGCTTGGCCTAATGCCTACTAATGAGGTAAAGAACTAAAGAACCT
CTGTGATTTCAATGAAGTCCCTTCAGATGTTATGGGCTACTTGTTACTGACAAGTATGGTAGGAAC
TGTAGGTCAAGCTGTCATAGGCAAATAGATCTTGCTGAAGAGGAAGAATTATTGGCTAA

| | |
|---|---|
| Sequence 1240 | cMhvSA033e07a3 |

ACTTTTTTTTTTTTTTTTTTTTTTTNNNNGAAAAAAAAAAANTTTTTTTNGGGGGCCNNNTNTNGGG
GGGGGGGNAAAAAAAAAAAAGNTTTTNNTNNTGGGGNNNAAAANCTTAAAANCCNNGGGGGGG
NNGNAAAANGNAAAAATTTTNTTTTTNNAACCAAAGGGCNANNAAAGGGCCNGGGGCNTAANNG
GGGAAAAGGGGCCCCNAAAANCCCTNGGGGGGNGGGGGGGGNNGCCNAGGGNAAANGGTTNTT
NAAAANGNCCTTTTTTTCCNAGGGGCANGGNTNTTTNCNACCNNGGNCNTNNCNAAAANNAAGGG
NTTNGNCCNNAANCNTTTTTTTTTTTTTTTNGAANCCNTNCNAAAAANTTTTTT

| | |
|---|---|
| Sequence 1241 | cMhvSA059b05a1 |

CCCTTTCGAGCGGCCGCCCGGGCAGGTACTTTAATTAGAGACGAGCCAGTGCAGAATAGCTGGAC
AGGCAGTGCGTCCACCCAGCGAGCAGACTGCCCAGGGGGGGCAGTCTCCACCTCACTGATGCAAC
TGGTGAAGGGACAGACAGGGGCGTGGATACATTTCTTCCTTCCCCAAAACAAAATGGGAGGATGC
GTGTGGGTTGGTGGGTTACAGAGAAAGATTCAAACATCATTCTGGCCTGATCAGTATTCTGGCAGT
TTACCATTATACATACAGAAAAAGAACAGAAAGTGTGTTAAAGAATCCAAGTTTTAAGGGGAACA
GAAAACAAAGTCATCTGCACTATGGAAGCCTATTTTTTTCTTTCTTTGTTTCCCCTCCTTTTTCTNTC
TCCTCCTCCTTTTTTCTT

| | |
|---|---|
| Sequence 1242 | cMhvSA002f05a3 |

AGTCGACCCCGCGTCCGCCAGATTTGATAAAACTGCATGATTCCTTAGGAGGAAGTGGAACCAGA
TGGACAAATAGAGCCCTCGTGTGATTGTTTCCTGCAGGAACACCAGATTGAACAACTATTCATGCA
AGAAAACACCTTCGTAGGAGCCAAAACAATTAGAGTGATCACAGTGCCTGATCTGAACATAATAT
TAAGGAGAGAGGAATTGAAGAGGATAGGAAAGACGGTCTTGCATTGCATGCACCATCCCTCCCTC
AAACCCAAGCAGCAGAGCATGGAGAGAAAATCTGTGCTTAAGGGAGAGAGAGCAAAGCAAGAGT
GGGACTCGGTACTGTCGTATCACAGTGGAACATAGCAAAGGGCAGAATTCTGCTGGCACCCAGGA
CAGGAGCCTTCAGACCAGCCCTGGCCCACAGGGAAATTCTGTGCCCCATTGGGAGGAACCCAAGT
CACAGNCAGCTTCACCACTGACTAACTGAAGTGGCCTGGGACCCANAATAAATTTGAGTAGCAGT
CATGCCACAAGGACCACAGTCCTAGGGCAAGCCCTGCTGCTTTGCTGATCTCAAAAGCACTGGACT
TTGAGTGCAACTCAATGCAACACCAGAGCCCAAGAGACTGCCTGCATCACCTNCTCCAATTCANGC
AGTACAGCTNCAGGAGAGACTCCTTCCACTTGAGGGAAA

| | |
|---|---|
| Sequence 1243 | cMhvSA032e08a3 |

GGTACTTTTTTTTTTTTTTTTTTTTTTTTTAAATTTTTTTTTTTTTTTTTTTTTTTTTTTTTTTTTTTTTTT
TTTTTTTTTTTTTTTTTTTTTTNGGGGGGGGGGGGNNTTTTTNNNNAAAAAAAAAAAAAAAAAANN
NNNNNGGGGGNNNCCCCNNCCANTTTNANNNGGGGGGGGGGGGNCCCCNNNTTNNNANTNTTTT
TTTTAAAAANANCNNNCNTNNTTTTNNGGNNNNNNCNNNNTTNNAAAAAAAANGCCCCCCCCCCC
NNANNAAANANANGNTGNGNAANANCCCCCCNCNGNAAAAAAAACCCCNNTTTTTTAAAANANG
GGGGGGGGGNGNTTNNCCCNNCTCCNNGANGANNNNGGCCNNCCCCCCCNAAAAA

| | |
|---|---|
| Sequence 1244 | cMhvSA032e04a3 |

CCCTTAGCGTGGTCGCGGCCGAGGTACATTTCTGTTAAAAAGAAGGTTGTCTTTCCAGCCTTATGTT
TTGTAGTTTAATTTGTTCACATTCATTATAATCCATTATTTAATACATTTTTCTTCCATTTGATCATA
TTACTTGCTGATAGGAAGGACTGAGTTCATTTTCAGCGTGTCTGGCTTTTCCATTTCTGTGGCCTGG
GAAGGTGGGTGGCTACATCATCATCCATGGTCTCTGAAATATCCTGTGTTACCAAGGCCTGCTTGT
TCCACCAAACTGCTCCATAGGCAGTTGTGACACCCAGAAAGATGCTGATATGGTTTGGCTGTGTCC
CCACCCAAATCTCATCTTGAATTGTAGTTCCCATAATCCCCAGGTGTCTGGGAGGGGCCCAGTGGG
AGGTAATTGAGACATGGGGGCGGGTTTT

| | |
|---|---|
| Sequence 1245 | cMhvSA002h12a3 |

GCACTGTGACAAGCTGCACGCTCTAGAGTCGACCCAGCAATCTCCCTGCTGCTCCGTCGTCCGCCA
GGACGTGAAGCATTCCCGGGCGACGTTTTCTACCTCCACTCTCGTCTGCTGGAGCGTGCTGCACGT
GTTAACGCCGAATACGTTGAAGCCTTCACCAAAGGTGAAGTGAAAGGGAAAACCGGTTCTCTGAC
CGCACTGCCGATTATCGAAACTCAGGCGGGTGACGTTTCTGCGTTCGTTCCGACCAACGTAATCTC
CATTACCGATGGTCAGATCTTCCTGGAAACCAACCTGTTCAACGCCGGTATTCGTCCTGCGGTTAA
CCCGGGTATTTCCGTATCCCGTGTTGGTGGTGCAGCACAGACCAAGATCATGGAAAAAA

| | |
|---|---|
| Sequence 1246 | cMhvSA049d12a1 |

CCCTTTCGAGCGGCCGCCCGGGCAGGTACTTTTTTTTTTTTTTTTTTTTTTTNATTTTTTTTTTTTTTTT
TTTTTTTTTTTTTTTTTTTTTTTTTTTTTTTTTTTTTTTTTTTTTTTTTTTTTTTTTNNAANNGAAACCNN
TTTNANNAAAAAAAAAACTNCCCNAAANANNTTTAAACNTTANANCCAANAAAAAAACCCANCA
TTTAAAAATTTTTNCNTTTNGCCCCCNAAAAAAGGNAAAAAAAAAGGGGNCAAAGGNNCCCCATT
TTT

| | |
|---|---|
| Sequence 1247 | cMhvSA032c08a3 |

ACAGTAAGGAGCAGACAAGATGGTTCTGGCCAAGTGGAAAGCCCATTTGCATAATAAGATTAGGG
TGGGGCGACCAGCCTTCCCACACACAATGTAAATGTCACACCTGATCCAATCAATCTGTGGGCCCT
ACATAAATCAGACAGTGCCTTCTCAAGCTTGCCTGTAGAATCCAGTGCACTCTGCCACCAGCAGGT
CTTTCCTTTTCAGATACCTCTCTCTGGCAAGAGACAGACAGAGACGGCTGCTCTCCTCTCCCCTTTC
TTCTGCTTATTAAACTTTCCGCTCCTTAACCCATTCCATGTGTGCGTGTCCATGTTGTTAATCTTCTC
AGCACAAAATGACCAACCCCAGGTATTTACCCCAGACAATGATGCCACTTCACTTGTAGGTTCCTC
CAATCCACTTTTCTCTTCATGAAATTAGTGAGAACAAAACCACCCTTTTCT

| | |
|---|---|
| Sequence 1248 | cMhvSA062d05a1 |

CCCTTTCGAGCGGCCGCCCGGGCAGGTACATCTCCTGGCCCTCAGGTGTCATGGAATTTAGGTAGT
AGCAGCCTGAGGCTGGGGTCCTGGGCACCTGACTGAACATCTCGGCAGATTTCCTATTGCCACCTC
AGTCTGCCTGTGGCTGTTGCCGTCTGTCTCCAGTCTCAGTCAAAGAGCAAGGCACCCAGCCCAGGA
CAGCTCAACAGACCCAGCGATTTTTAAAAAGAAAGAGGAGTGCCAAAGCCACAACTCANAATTCC
AACCCCCGGGCCCTCACGTGACCTCGGGAACCAATGAGAGGAAGAGAGGAAAATGGGAACGTTT
GCAGTCAGCCCTAAGCCCCGACCAGAGGCAGTTCCAGCCGCCAGGGTCCCTCACACAACGCTGAA
AGCAAAATACACGTATTTGAC

| | |
|---|---|
| Sequence 1249 | cMhvSA031g04a4 |

CCCTTTCGAGCGGCCGCCCGGGCAGGTACTCTAGCCTGGGTGACAGAACGAGACTCTGTCTCCAAA
AACAAACAAACAAAACACAGAAATACTGGGAATAAAAGTATTTTTGAAACATGTAGATCCTCTTT
TATTAAGAAAGAGGCAGACATCTCACACTTAGGAAAATCTCAACCCTTAAAGAGAGAAATGAAAT
AGAAATTTTACAAATCAAAACAAAAGTAAAAAAAATCAAAAATAACAGATTTTTATCAAAGAAAT
TAAAATTTCAGATTTAACGTGGAAAATATCTGAAAAATGTTGTGATTATGAATAAGCCTTATGACA
TTTCTGCATCACTTCCTGACTTATAGACCTATTTCTCTAAATTGATATTCATTCTACACCAATGAAC
CACTA

| | |
|---|---|
| Sequence 1250 | cMhvSA031e12a4 |

CCCTTAGCGTGGTCGCGGCCGAGGTACCTGGGTGATGGCCATACTGCGTGCCGCCATAGCTCAAGC
CATGTGCCTGAGGCTGTGCATGAGGGAGAGAAAGAATGTCCACTCCCAAAAGAACTGATTCAGGC
ATGAACAGAACCATTGCACATCCTCAGGAGGTTCTAGCAAACCTGCACATCCATGTCTGCACTTAG
ACAACATAAACAGAGTGAGAATGCCTTCCCAGAGCACAGCAGAAGTTCAACTGGCAACGACCAGG
AGAATTTTCAGCTCATCCTTTACAGAAAATGTAACTTCCATGGAGAGGACAGGAGAATCAGACAA
AGACAAGCGGAGACTCTTTCTTTTCTGCACGTGCTGGTACCTGCCCGGGCGGCCGCTCGAAAGGG

| | |
|---|---|
| Sequence 1251 | cMhvSA031a09a4 |

ACACATGTCCAAGGTCAGGTCCTGGGTGGTAAAGGTAAATACAAATTGGAAGGGCACTGTGTGAG
CCAAAATGAGTCAGATTAGTCATGATTCATTTCCAGTTTGGGTTTTGGGTGGTCTTGGAGAATGTT
GTAAGCACTGCTTCATTGATAGGTTGATTGAGCCAGACTTTACTCAGCAGCCTGGAAAAGGAGAG
ATGGGCTCTGGGTTCTACCTTTGCTCACTGGTAAGTTGCTAAGATTTCAGCTTTGCCCTCAAACCCT
GAAGTAGTCCTTCATTCACACAGTGGGATCACTCGAAAATGTCAGATGGGGAAGTCCATAGGTTGT
TACTTTAAAGAAAATAGAAAAAATGCTGGAAAAGGTTTCTTCAATTTTAATACCCA

| | |
|---|---|
| Sequence 1252 | cMhvSA002a01a3 |

ACTTTTTTTTTTTTTTTTTTTTTTTTTTTTTTTTTTTNTTTTTTTTTTTTTTTTTTNAAAAAAAANTTTTT
TNNGGGNANNNNNNNTTTTTTTTTTTTTTTTTTTNNAAAAGGGGGGGNNAAAAAAAAAAAAAAAN
AAANGGGGGNNAANNNNAAANGNTTTTTTTTTNNNNAAAAAAANNGNNTTTTTTTNAAAAANTTT
TTTTAAAANNNNAAAANNTAAAAANNTTTTTTNNNNNGAAAAAAAANTTTTTTTTTTTTTNGGGGG
GGGAAAAAAANANNANTTTTTTTTTTTNNNNNNNAAAAAAAAANNNTNNAAAAANGGGGTTTTTT
TTTAAA

| | |
|---|---|
| Sequence 1253 | cMhvSA057d07a1 |

CCCTTTCGAGCGGCCGCCCGGGCAGGTACGCGGGGGCCGAGAGTCTGTGCGAAGGTCCGTGGACA
GACTGCTTTGCCTGTTGTTGCTCTTCGGAGGCGGCGATCCCCGAAGGCGAGCTGAAATACGGCTGC
AGGCTACAATTTGCAGCCGACCATTATGGATGACAAGGAGCCGAAGAGGTGGCCCACCCTCAGGG
ACCGCTTGTGCTCGGATGGCTTCTTATTTCCCCAATACCCCATTAAACCGTATCATCTGAAGGGGAT
CCACAGAGCTGTCTTCTATCGTGATCTGGAGGAACTGAAGTTCGTTCTGCTCACGCGTTATGACAT
CAATAAGAGAGACAGGAAGGAAAGGACCGCCCTACATTTGGCCTGTGCCACTGGCCAACCGGAAA
TGGTACCTCGGCCGCGACCACGCTAAGGGCGAAT

| | |
|---|---|
| Sequence 1254 | cMhvSA058h03a1 |

CCCTTTCGAGCGGCCGCCCGGGCAGGTACTTTATTTTTTTTTTTTTTTTTTTNNATTTTTTTTTTTTTT
TTTTTTTTTTTTTTTTTTTTTTTTTTTTTTTTTTTTTTNTATTTTTTTTTTTTTTTTTTANGGGGNTTNAA
NNNAAAAANTTANTTTNGNGTTTTNAAAAAAGGGGNAANTTTTNAAAAANNGNGGNNAAAGNTN
TNAAAAAAAAAATAAAATTTTNNGGGGGGGGGGGNGNNNGTTTANAAAAAANTTTTNTNAAAAA
NNTNTNAAAAAAATTTTTTTTTTTTTTTTNCCNAAANNNTAAAAATAATATTTTTNAAAAAAAAAN
NNNGNNNANNCTNTTNTTNANAAAAAAAAAAAAAAAAAANNNNGAAAAAAAATATTTT

| | |
|---|---|
| Sequence 1255 | cMhvSA033a02a3 |

GGTACCATTGGTGGCCAATTGATTTNATGGGGAGGGAAGGNAACGCCTGGCTCGGAGCAGTAGCC
TCTGAGGTGTCCCTGGCCAGTGTCCTTCCACCTGTCCANANGCATNGGGGAACATTTTCACCAACC
TNTTCAAGGGCCTTTTTGGCAAAAAAGAAATGCGCATCCTCATGGTGGGCCTGGATGCTGCAGGG
AANACCACGATCCTCTACAAGCTTAAGCTGGGTGAGATCGTGACCACCATTCCCACCATAGGCTTC
AAC

| | |
|---|---|
| Sequence 1256 | cMhvSA059h05a1 |

CCCTTTCGAGCGGCCGCCCGGGCAGGTACAGCTGGTCCAGGATAGCCTGCGAGTCCTCCTACTGCT
ACTCCAGACTTGACATCATATGAATCATACTGGGGAGAATAGTTCTGAGGACCAGTAGGGCATGA
TTCACAGATTCCAGGGGGGCCAGGAGAACCAGGGGACCCTGGTTGTCCTGGAATACCAGGGTCAC
CATTTCTCCCAGGAATACCAGGAGGGCCCTAAAAAAGAGATAAAAATAAATTAAATAGTGAAAAA
TCCTGGTGATTCACAATCATTATCAGATTGTTGTTTCTCTACTTTATAATATTAGGAAACAATATAA
GTAATATATTTTCTTTATAACACATACTTTTTAATCAAAATCTTGTGAATAATTTAAGTATAATGTA
TTCCTTTGT

| | |
|---|---|
| Sequence 1257 | cMhvSA010h06a3 |

CCCTTTCGAGCGGCCGCCCGGGCAGGTACGCGGGGAGTGCCGCCGGGACTCTTGGCGGGTGAAGG
TGTGTGTCAGCTTTTGCGTCACTCGAGCCCTGGGCGCTGCTTGCTAAAGAGCCGAGCACGCGGGTC
TGTCATCATGTCGCGTTACGGGCGGTACCTCGGCCGCGACCACGCTAAGGGCGAATTCCAGCACAC
T

| | |
|---|---|
| Sequence 1258 | cMhvSA003c05a3 |

ACTTTTTTTTTTTTTTTTTTTTTTTTTTTTTTTTTTTTTTTTTTTTTTTTTTTTTTTTTTTTTTTTTTTTTTT
TTTTTTTNNTTTAAAANTTNNTTNNNANAAAAAATTNTNNANNTNTNTNAAAANAAGAAAGCTTTT
TTTAAAAAAAAAAAAAAAAATTTANCCNGNCTCACAAATGTAAGTANANAAATNTNANGNNTAA
AAAAAAANTNNCCNCTCCTTNNTNTTTAAGGGGNAAAANNCCTTTTNCNTNNGNGNGNAAAAAAA
AAAATTNNNTTTTTNNNGNANACTGGCCGGCNATTTCTAANGGAANNTNGNTNTATNCTNAAAAA
AAATAGNTATTNNNGGGAANAAAAAAAANNAAANAAAATTNNNNNNGGGAACNANAAAAAAAA
AAAAAAAANNNNCCCNCCNCNNNNNAAAAANANTTATNNNNNCNNANNANANNNANGANAAAA
NATTTNNNTTNNNAAAAAAAAAAAAAAAAAAANNTTNGGGGGGGGNNGNNANAAAAAAAAANA
AA

| | |
|---|---|
| Sequence 1259 | cMhvSA018d11a3 |

CCCTTTCGAGCGGCCGCCCGGGCAGGTACCCCTCACCCTCCCTCCTCCAATCTCCCCATGGCAAAA
AAATGCACCTTTTTTTTTTTTTTTTTTTGNAANGGGNTTTTTNTTTNNTNCCCNAANNGGGNNGNCG
GGGNCCNANTTTANNTTANNNGAAGCCCCNCCNNNNGGGNTNNNCCCNTTTNNCNGNCNNAACCC
NCNGNNGNGGGGGGNANANNGGNCCCCCCCNCCNNNCCNGGGNAATTTTTTNNNTTTTTNNNAAA
AANGGGGTTTNAANGGNCNTNCCNNGNNGGGTTTTNTTNCCCCNCCNTANNANTTNNCCCCCTTG
GNCNNCNAAAGGGGNGGGNNTAAAGGGCTNNACCCCCNNCCCCNACCAATGGCCCTTTTTTTTTT
TTTTNAAAAAAAAA

| | |
|---|---|
| Sequence 1260 | cMhvSA031d07a4 |

GGTACTTTTTTTTTTTTTTTTTTTTTTTTTTTTTTTTTTTTTTTTTTTTTTTTTTTTTTTTTTTTTTTTTTTT
TTTTTTTTTNNNNNNNNNNAAANNNTTTTNNAAAAAAAAAAAAAAAAAAANNNNTTTTTNNNATN
GCANNGGGGGGGNNNNANAAAANTNTTTNNNNTTNAANNNCNNNNNNAAAAAAAAAAAAAANA
GGGGGGNGGAANNNTNTTNCTNANAAAAANANCTTTTTTTTTTNGCNNNAAAAAANAANACNCCN
CCCNCCNCTNNNGGGGGGGGGGGGGGNGAAANACCCNNGGGGGGAAAATTT

| | |
|---|---|
| Sequence 1261 | cMhvSA031e07a4 |

CCCTTTCGAGCGGNCGCCCGGGCAGGTACTTTTTTTTTTTTTTTTTTTTTTTTTTTTTTTTTTTTTTTTT
TTTTTTTTTTTTTTTTTTTTTTTTTTNTAANAAAAAAAANANNTTTTTTTTTTTNCGCNGAGNNNNNN
TNAAAAAAAAAAAAAAANNNNTTTTTTNNGGGGGGGGNNNNNNNAAAAAAAANNTTTTTTTTTTT
NGGGGGGGGNACNAAAAAAANGGGNGGANNAAAANNNTTTTTTNTCTNGNNANANNNAANNCN
NTAAAAAAAAAAATNNNNCNNCACTTTTTTGGGNGANTGTAANGGGGGGGNGGGGGGG

| | |
|---|---|
| Sequence 1262 | cMhvSA037g01a3 |

CCCTTAGCGTGGTCGCGGCCGAGGTACTTTTTTTTTTTTTTTTTTTTTTTTTTTGGGGTTTTTTTTTTT
TTTTTTTTTTTTTTTTTTTTTTTTTTTTTTTTTTTAAAAAAANCCCCNNNNTTTTTTTNTGGGNNNAAA
AAAAAAANANNCCCCCCCNNGGGGNGGGGGGGGGGGTTTTTNNNNCCCCTNNTGTTTCNNNNAN
NCCCCCCCCNCCNNNTTTTTTTTTTTTTTTTTTT

| | |
|---|---|
| Sequence 1263 | cMhvSA054a06a1 |

NGGGGCCCTTAGCGTGGTCGCGGCCGAGGTACTTTTTTTTTTTTTTTTTTTTTTTCTCAAGCGACGCT
CANACAGGCGTAGCCCCGGGAGGAACCCGGGGCCGCAAGTGCCGTTCGAAGTGTCNATGATCAAT
GTGTCCTGCAATTCACATTAATTCTCGGNGCTAGCTGCGTTCTTCATCGACNCACGAGCCGAGTGA
TCCACCGCTAANAGTCGCCCGCGTACCTGCCCGGGCGGCCGCTCNAAAGGGCGAATTCCAGCACA
CTGGCCGGCCGTTACTAGTG

| | |
|---|---|
| Sequence 1264 | cMhvSA003c10a3 |

CCCTTAGCGTGGTCGCGGCCGAGGTACTTCACTGCGGACTTGACTTCTTGAGCAAGAAGGCTGGCA
CTGTTCATTAAGAGAATCACAGAGATGAATCTCACAATGCAGGAAAACTAGGTCATAATGTCCAG
CAAACATGAACATCTGAACTGAGAACCGGCTTTCCGAGGACTGCCCATTCTCCTCCACGTGGATGG
TGGAATCACGCTGATTTGAGCAGCTGTTTCTGATGATGAAAATACTTCACAAGGTNAGCCTTGTCT
TCAGTGGGGGGTGGCATTAGCAGTTCCTCAACACCCAGGGTTAAAACCCGGGGAGGTGTCCCCTT
GTTCCAAGATGGCACCCACATTACCAGCACCGGGACCTCAACAGACAGTTTCCAACTGCATCCCCT
TTCGTAAAGGGATTCCGGTGGTTAGTTTTCTGGGTCTTTGGGGAAAGAANGGGCCCATTCCCTGGA
CCAAATTGAAAACTTCTTTCCATTTTCCCCCGGTCCCACCACCTTGGACCGTTTTCCAAGGGGGAA
ACCTTTACCAAATTGGGGGCCTTGGCAAANGGGCCAAGCCTTTNGGGAANGGCTTGGACCTTTTCC
ATTGTTCCCCAGGTGGGGGGTAAGGGGCCNCATTTTGGGAAAAGGTTTGGAATGGTTTGGAAGGG
AATGGGGTGGTCCTTCTTGNNTGAATGGAAAAATTNCATTTGGNCCCCAANGGGAGAAGGGGGNG
GTTTT

| | |
|---|---|
| Sequence 1265 | cMhvSA018f03a3 |

CCCTTAGCGTGGTCGCGGCCGAGGTACGCGGGAACGTGGTCCCTANAACAAGAGGCTTAAAACCG
GGCTTTCACCCAACCTGCTCCCTCTGATCCTCCATCAGGGCCAGATCTTCCACGTCTCCATCTCAGT
ACCTGCCCGGGCGGCCGCTCGAAAGGG

| | |
|---|---|
| Sequence 1266 | cMhvSA004g09a3 |

CCCTTTCGAGCGGCCGCCCGGGCAGGTACATCTGCCAGTGCTCAGAAGGTCCAAGTCTCAATCCAG
ACCCCAGCAGGTCAAGTTCTCCGATGATGTCATTGACAATGGGAACTATGACATTGAAATCCGGCA
GCCTCCGATGAGTGAAAGGACTCGGAGACGCGCCTACAATTTTGAAGAGAGGGGATCCAGGTCTC
ATCACCACCGCCGCCGGAGAAGTAGAAAGTCCCGCTCCGACAATGCCCTGAATCTTGTTACAGAA
AGAAAATACTCTCCCAAGGACAGACTGCGGCTGTACCTCGGCCGCGACCACGCTAAGGG

| | |
|---|---|
| Sequence 1267 | cMhvSA003d12a3 |

ATTCGCCCTTAGCGTGGTCGCGGCCGAGGTACGCGGGGGGAGAGCAGAGCGCGGCGGCTGGAAGC
TGCTAAGTCAGAGCCGCGATGTTCCGGATTGAGGGCCTCGCGCCGAAGCTGGACCCGGAGGAGAT
GAAACGGAAGATGCGCGAGGATGTGATCTCCTCCATACGGAACTTTCTCATCTACGTGGCCCTCCT
GCGAGTCACTCCATTTATCTTAAAGGAAATTGGGACAGCCATATGAAGGACAGGGACATCACATT
ATGAAATGCACCGATTATTGAAGGAGCCCTGGGTTACAGGTTTCCGACTCCTCTCTGCCAAGGTGA
ATAAGGCCCAGNAAAGGGTGGTAAAGGAGACTCTTTGAATGGGACCATTAAAAATTTCTTGCTTG
TTAAANAAACAAGTTTNGGCTCTGGTAACCTGGACCTTTCAAAAGNCTAAAAATANTAAAAACTT
NTTTTTGGGGAAGGTATTGAAAACGATTGTCCTCGTGGATCTGGTGTACCCTGCCCCGGGGCGGCC
GCTTCGAAAAGGG

| | |
|---|---|
| Sequence 1268 | cMhvSA031e01a4 |

GGTACACATGCCAGCTCTGGCAACTACCCTATGCTGGCTCTACCACCAAAGACCCGGAACCAAAG
TTGGGTGCACAGTTTGCTCCCTGAATGGTGGGCTCAGGCACGGCTCTGACTTCATTTCTCAGGCAG
GCAACAGACACGTTTACCTTACGCTCTGGCTCCTGCTGTTCCTTGCANCAAGGGGGAATTCGATGG
GACCTAAAAATCATCTGGAACATACACAGACATGGATATCTTCTCTCTCACATAAACACAAAGACC
TTTCCCCATATTTCCGTGCAGGCCAAGCCTCTGTATTTTCCAGCATGACACTGTATTTGCGTATTGT
AGTGGATGGGACATTGGGGATCTCCTAGTCCTGT

| | |
|---|---|
| Sequence 1269 | cMhvSA062h11a1 |

CCCTTCGAGCGGCCGCCCGGGCAGGTACGCGGGTAATTTGGTTGGCCAATTAGAAATGCCTTTTTC
AGTTGGTGTATTGAAAGCTTTCCTTTAACATTTTCACCTGCTCATTGTGATTCCTCCTTTTAGTCTAA
TATCTTTCCAGGTCATACTTGTTTTTAATCATTAAATATTTTCTTCCTGGTTTTGGAGACTAAGCTGA
TAAACTTTTTTTAAAACTTAAGCATTGTCATTGCTATTTTTTTTAATTTGACTTTCCTAGGAGTTTAA
GATCAGCCATGACCAACATGGTGAAACCCCATCTCTATTAAATACACAAAATAAAAATGAGCCAC
CGTGCCTGGCCAGAATAGGTTTTTTCTTTCAACTTGATCAGTAGAAAATGGACATCAAGT

| | |
|---|---|
| Sequence 1270 | cMhvSA062e09a1 |

CCCTTAGCGTGGTCGCGGCCGAGGTACGATGTTGTGTGGGGAGAGGTGATATGGTCACTGTAGGG
AGACGGCACATGCTCACTATCATAATGGCTTCCATGGGGTGAGGAGTGTGAGTGATCACTGCTGTA
TTGCTGTCGTGAGGTGATTAGGTCATCTGCCTTGCTCANCAGCTGGGCAGGATGTGGCCTCTGGGA
GGCATGGCTGCCGTCATGAAGTCCATGAAACTGTCCTGGGAAGGCTCTCTCCCCAAGTGCACTCTG
GCTGATCAGAGTGGCAGAAATAAAGGCCAACGTTGGCTGGGGCAGANAACTGCCCCTGGATCTNN
CCTGCCAGGGGTGTTANGTGGGTTTGACAAGGTNNCAGAACGGNCAGGTTCTTATCCANCTNTAG
ACTAGAAAAATTATC

| | |
|---|---|
| Sequence 1271 | cMhvSA057d11a1 |

CCCTTTCGAGCGGCCGCCCGGGCAGGTACGGGGGTTTGGTTGACTGCCAGCCCTGGAGGGTTGTCT
TCTGCCCACACCTTTGACCATCACTTAGCCAGAGCTGGTCTTATCTCTTGACCTGGCTCGGTTAAGA
AAAGTCTTCATTCCTCCTCCTGGGGGACAGTAAGGGCCATGATGACTCCCTTTCCGGGTAACTTTA
GCTGTAAAAGAGCTGTGCTCTGTAAGAGAGATGGTGGCTCTCAGCTTGCTAAGCAAGTCCCTTCCC
AGCAAGGGCAAGGAGAAGTCGGGCATGTACCTCGGCCGCGACCACGCTAAGGG

| | |
|---|---|
| Sequence 1272 | cMhvSA009c03a2 |

GGTACTTCCCATAATCCCCACATGTTGTGGGACGCACCCGGTGGCAGGTAATGGAATCATGGGTGG
TTACCTCCATGCTGTTCTCATCATAGTGAGTTCTCATGGGATCTGATGGTTTTCTAAGGGGCTTTTC
CCCTTTTTCTCAGCACTTCCTTTTGCTGCCATCATGTGAAGAAGGATATGTTTGCTTCCCCTTCCACC
ATGATTGTAAATTTGCTGAGGCCTCCCCAGCCATGCGGAACTGTGAGTCAGTTAAACCTCTTTTCTT
TATAAATCACCCAGTCTCAGGTATGTCCTTATAGGAGCCTGAGAACAGACTAATACACCTGTCAAT
GCCAAAATGATATATTTTGGTGGATTTAAAA

| | |
|---|---|
| Sequence 1273 | cMhvSA002d03a3 |

ACTATTAGGGGGAAGTTCNGTACACACAGGGCCGTANTNGGGNGNCCCCTTCTTAAGAATGGCCA
TTGGCCNTCGGANGCCGGGCCCGGCCCAAGTTGGTGGAATGGGGAATATTCTTGCCAAGAAATTC
CGCCCCCTTTAAGCCGGGCCCCGCCCCGGGGCCAAGGGTACCCGGCGGGCCCCGTTTAAAAACAT
TGTTGTTCAACTTGGGGGCCAAGGCCGGGTGGCCCCTCTTAAATAACTTGGGTGGAATGCCTTAAG
NAAGGGTTGAATGGTTTTTTTGGGTTAAAACAAGGGCCGGGGGGGTAAAAGAATTTGGCCCGGAA
GTTTCCCTTTTTTAACTTTTTTTTTTTAAACCCTTTTCCCTTTTAATTGGAAGCCATTTGGCCCTTGGT
TGGTTTGGGGGTTTTGGACCAAGTTGGAAGGGGGGTTAAATTAAATTGGACCTTTTGGTTTTGGGG
TTTGGAATTTGGTTAAGGAATTAATTTTGGGGGGCCTTGGTTTAAAATTTNGGTCCAAGGTTTTCCA
AGTNGGTTTTTTNAAATTCCTTGAACCGCCAAGGGCCTTTTAATTGGCCGGGGAAGGGGAAGAAA
AATTGGTTTTTTTTCCAATTGGTTTTAACCTTTTAATTAACTTAAAACCATTTTAAGGTTTTCCTTTT
CCTTAATTAAGGGGGGTTGGAATTAAGGAATTTTGGGGTTCCCCAAAATTTTGGGGGGTTGGTTGG
AAGGGGAAAGTTTTCCAAAGTTTTAATTAATTGGTTTTTNGGGGGGAATTTTTTTTTTTTAAGGGGT
TAAAGNTTGGGGGGTTGGTTTTGGAAAGCCCTTTTGGAAAACCCGNCCTTTTTTCCTTTTAAAATTT
NGGGGTNGGGGCCTTGGCCTTTTTTTTAAGGGGCCCCTTACCTTTAATTGGGGGGTTGGTTTTAAA
AAATTTTTTTTTTTAACCTTCTTCCTTCNTTAACCAAAAGGGGTTTTTTTTTTT

| | |
|---|---|
| Sequence 1274 | cMhvSA003b05a3 |

CCCTTTCGAGCGGCCGCCCGGGCAGGTACGCGGGACACATTCAGAGGTGAGCCCAGAGCGGGTAA
AGTGGACTGGGGAGAACTTCGGAGGATGTTCATGTCCAGGAGCAGCCCCACGCCCTGTATGGTCG
GTGTCTAGAGCCTCACAGCAACTAAGACCAACCCAGCTCTCAGAAGAAGGAATGTCAAAATGTCA
TGTTCAATTTTACATTCAGTGCCTGGAATCTTTTCTTCACAATTGAAATGAAATGTGCTGAAGGAG
GTGAATCCATGCATTAATCTTCAGCTCACAAAGGAAATACTACATAAGAAGCAAGACCACAGACT
CAAGACGGACATAATTGGATTTTTTTTGCCATGGCCTGGAAAGAAAGGTACCTCGGCCGCGACCAC
GCTAAGGG

| | |
|---|---|
| Sequence 1275 | cMhvSA002c09a3 |

CCCTTTCGAGCGGCCGCCCGGGCAGGTACCTATTAACATCACTCAGCTGCTGTGAAATAGGCTTAC
AGGCAACATGGAGTGTCAATTACCCAATGTTTAAAGTCGATCATACAGATTGGACTACAATCTCTA
TGGCTCATAAAGTCTTTAAAGGATTGACAGATGATTTATCTCATATGTAGACAATGATTCTCAGCA
GTTAACTAGCGCAACTTGATAATATCAATTGCTTGAGAAAATCAGATAATTGCTTGAGAAAATTAG
GACATTGCTTGAGGAAGTTAGGTAGTTAAATAAATTACTTTTTTTAAAGAATAGTTTAATATTTTGG
CAAGTAGACTTTAAAATAGGTTGGTAATATTTTAAAGGCTACTTTTAAAGAAGTAGCAATATAACA
TGTTTAATTATGAAAAATAATGTTGGAAACAATTCAATTTTCTATCAGATCATTCACAAATACAGA
AATACCATCTCAATAATTAGAAGAAGTAGCAGCAATTTCTGTCATTTTTATGCCAGTTACTCTTAGT
CCATTTATTTG

| | |
|---|---|
| Sequence 1276 | cMhvSA031h09a4 |

CCCTTTCGAGCGGCCGCCCGGGCAGGTACGGGGAAAAGTGATGACAGCGTGACTATGTAGAGTTA
TATAAACTATGTAAAAAGTCATAAAAATGTGAGTGGAGTGAATTTGTCACCTCGATTTTCTTTTCC
CTTAACCACTCTACTTTCCTTCTCTCTCCATCTGTAATGCTATGCAGTAACTTCAGTTTTATGCTTCC
ATCCATGGCAGATATCATCAAGCAATCTAACACTTATTCTTGTTGAGGTTCCAGTAAGCCTTGAGT
CCAAGCTGCCACTACTACAGGGGGTTATCCACATGGAAAGTGCAGATTGTTACTACTCACCTCATT
CCGTAAGCAGAAGCAAATTCTGTATAGATGAAGGACTTAACTATGACAGCCAATACTTTAAAATA
TTTAGAAAATAAATATTTTTATTATC

| | |
|---|---|
| Sequence 1277 | cMhvSA057c11a1 |

CCCTTAGCGTGGTCGCGGCCGAGGTACGCGGGGAATTGCTAATGGGAATGGGGTTTATTTTGAGGT
GATAGAAATATTGATGAAATTAGAAATTGGCGGTGATTGCTAATGGGAATGGGGTTTATTTTGAGG
TGATAGAAATATTGATGAAATTAGAAATTGGCGGTGATTGCTAATGGGAATGGGGTTTATTTTGAG
GTGATAGAAATATTGATGAAATTAGAAATTGGCGGTGATTGCTAATGGGAATGGTGTTTATTTTGA
GGTGATAGAAATATTGATGAAATTAGAAATTGGCGGTGATTGCTAATGGGAATGGGGTTTATTTTG
AGGTGATAGAAATATTGATGAAATTAGAAATTGGCGGTGATTGCTAATGGGAATGGNGTTTATTTT
GAGGTGATAGAAATATTGATGAAATTAGAAATTG

| | |
|---|---|
| Sequence 1278 | cMhvSA003a10a3 |

CCCTTTCGACGGCCGCCCGGGCAGGTACGCGGGGAGAGACAAAAACAGAAGAGGGGAAACATGT
TTCCTACTGACGACAGGTGATTACACGTGTGCTTCTGATGGAGGGATCAGGAAAGGATATGAAAA
ATCCCGAAGCTTAAACAACATAGCGGGCTTGGCAGGCAATGCTCTGAGGCTCTCTCCAGTAACATC
ACCCTACAACTCTCCTTGTCCTCTGAGGCGCTCTCGATCTCCCATCCCATCTATCTTGTAAACCAAA
CAACCAAACTGCATCAGTCGGCTAAATTGTATTAATTCAAGTGCTGTTTACCCCATAATGGAAATA
ATTAAATGTAGAGTTACTCCAGGCTCCATTAATACAGTATAAATCTTGCATGATACTACAATTTGA
AGTCAGAAATGCCACTTGGGTAGCTAATGAATCTTACCCAGGCTTTAAAGATTGTCTAAAGTAGTG
CTAAAATCCCTCCTATTAATTGCCCTGATATCCTTTTGCAATAAAA

| | |
|---|---|
| Sequence 1279 | cMhvSA002d05a3 |

CCCTTAGCGTGGTCGCGGCCCGAGGTACATGCCTGTAATCCCAGCTACTGGGGAGGCTGAGGCAG
GAGAATTGCTTGAACCTGGGAGGCAGAGGTTTTAGTGAGCTGAGATCCCGCCATTGCACTCCATCC
AGCCTAGGTGACAGAGCGAGCGAGACTCCATCTCAAAAAAGAGAAAGAAGAAGAAGAGAGCTCA
ACAATGCAGCCAGGGAAGATTTCCTGTAGGAGTCTTGAGACAGGAGAAAGAGAGATGGAAGAGA
AAGAAAGCGCATGCTGCCTCTGAAAAAATGGAGAGATCACCCCCGCGTACCTGCCCGGGCGGCCG
CTCGAAAGGGCGAATTCCAGCACACTG

| | |
|---|---|
| Sequence 1280 | cMhvSA049h10a1 |

CCCTTTCGAGCGGCCGCCCGGGCAGGTACTTCCGATCAGCCTCCTACAAACCCTCTGCTTTCAGTCT
TCAAGCCATTCTCCACACAGAAGCTGGGAAGAGCTCTCAAAGGCAATGCCAACCATGTTCCTACCC
TGCTGAAAACCTCCCAATGAGTTAGGATGTTAGGCTCTCAAAGCACTTAACAGCCTAACTCCATCC
CATGACCTCGGGCCCTCCTTGCTCTTTTCCCACCTTTCCCTCATTGCTTCTTACCTCGGGTCCAGCCA
CAATGGTTTCCTTTCTGTTTCCTGAACAACTCAGACCTTTTCCAGTCTTAGGACTTTTGCTGTTGTTC
TTTCTGCCTGAAGCCTTCTTTCTGCCAGCTCTCGGCATGCTTTTCTT

| | |
|---|---|
| Sequence 1281 | cMhvSA058c10a1 |

CCCTTAGCGTGGTCGCGGCCGAGGTACTAGCAGAATTCAGCTCCTGCAGTGATAGGACTGAGGTCC
CTGTTTCCTTGTTGGCTATCAACTGGGGTTTGCTCTGGGCTCCTGGATACTGCTGCATTCCTTGCCA
GGTAGTCCTCTCCATCTCCAAGCCAGCAACAGCACATAAACCCCTCTCCTGCTTCGAATCTCTTACC
TCCTCAGCTTCTGACCTCTAAATACAGGTTTAAAGGGCTCTGGCAAATGGGTCAAGCCCACTGACA
ATAAATTCCCTTCTCGAAGTCAACTGTGCCATATATTAAACATAATCACAGGAGTATAAGCCACCC
TAGTCACACAGCCCATGGATTATGCAATATATACTGGTAGTGGGTCTACTGGAGGTCATTTANAAT
TCTACCTACCACAATTTACAAGGAAA

| | |
|---|---|
| Sequence 1282 | cMhvSA018c01a3 |

CCCTTTCGAGCGGCCGCCCGGGCAGGTACAAGGTTGGTAGGAGGAAGAGAAGAAATGATTGGCTC
CCAGAGGCTTCATGGGCTCCCAATTCATGATTCTTTCTCTGTGGCTAATTTTTGTTAAGTATAAGAA
TTCCAGGAATCTCTTAGGAATTGTGGAGACTGCTTTCTCCTGAAATATAAAACATCTGCTCTTGGTC
TGTTTGGCGCTCCACTGTCTGAGGGGAAAACAGGGAAAAAGAGGTAATATAAAACAGACATTGTT
TCAGACAATAAATCCCCCTTTACTCATTAATGAGAAAATAAATTTAGGGCCAGANGTGTCAGACTT
TTCANGANGCCTCTTTGTCTTTTCTTTTCTTTTTTTTAATAATTTAAAAAAAG

| | |
|---|---|
| Sequence 1283 | cMhvSA031e06a4 |

CCCTTTCGAGCGGCCGCCCGGGCAGGTACCACCTATGAAGTATTCTGCCTAAAGATATTAAACCTG
AAGCTTATCAAATCTGTAAATCTGACTACGACTTGACTGAAAATTTAGTGGCAAAGGAATATAGTA
AATGACATCACAAGGATATAGCATCCAAACCCAGAAAGCGGATATTCTTTAGGATAAATGACCCA
GTTTCCTCAACAATGAAATGGCCTGGAATAGAAAAAAGAGGGAGAACTTAAAATAACATACCAAC
CAAATATAGCACATGGATCCTGTTTTAATATGGATTCAGAAATCCAATTCTGAAATGACATTTTTT
AAAAATCANGAGGCCGGGCGTGATGGCTCATGCCTGTAATCCCAGCACTTTGGGAGGCTGAGGTG
GGCGGATCACAAG

| | |
|---|---|
| Sequence 1284 | cMhvSA041b10a3 |

ACCAAAACTTGTCCGAAAATTATAGCTAAAGTTTTCTCACTTTTCCTGTCTTTCTCACTACTGGGAA
GGCATTAGGAATGGAATTATCTGAGCATGCAGAATTGTGTTTTATTTGCAATAGGTGAGTATTAAC
AAAAATGCATAGGTGTGCATCTATAAAATTTATCATATACACTCAGTATAGACAAATACTTATGAA
ACATTAGAAAATCAGCTGAATACCTTGTTAATACACAGTATCATTCAGCATAATTGAGTTTCTAAA
TTTTAATAAGTTCTCAGGCGATGCTGATACCAGTGGTACC

| | |
|---|---|
| Sequence 1285 | cMhvSA003e11a3 |

CCCTTTCGAGCGGCCGCCCGGGCAGGTACGCGGGGACACATTCAGAGGTGAGCCCAGAGGGGGTA
AAGTGGACTGGGGAGAACTTCGGAGGATGTTCATGTCCAGGAGCAGCCCCACGCCCTGTATGGTC
GGTGTCTAGAGCCTCACAGCAACTAAGACCAACCCAGCTCTCAGAAGAAGGAATGTCAAAATGTC
ATGTTCAATTTTACATTCAGTGCCTGGAATCTTTTCTTCACAATTGAAATGAAATGTGCTGAAGGA
GGTGAATCCATGCATTAATCTTCAGCTCACAAAGGAAATACTACATAAGAAGCAAGACCACAGAC
TCAAGACGGACATAATTGGATTTTTTTTGCCATGGCCTGGAAAGAAAGGTACCTCGGCCGCGACCA
CGCTAAGGG

| | |
|---|---|
| Sequence 1286 | cMhvSA010a03a3 |

CCCTTAGCGTGGTCGCGGCCGAGGTACAGAACCCAGGAGATCCCCAGTCCCTGCGATGTAGGATC
CCGGACCCCCGGCGCTAAGTGGAGATGCGCCAGCTGCCCCACACTGGAGAAGGCTCAAAGAAAAC
AAATCCCACCCTTCGCCGCAGGTGGATTCTCCTCCCCTAGAGCTACTGTCCAGTTGCTACTGGCCTC
CAGCAAAACAAACATCAGTATGGACGGAAGGAGCAGGACGCAGGGTGGGGAGGGTCACCTTTCT
GGGAGAAAAGAAAGNCCGCGGNCTANCGTACCTGCCCGGGCGGCCGCTCGAAAGGG

| | |
|---|---|
| Sequence 1287 | cMhvSA033d05a3 |

CCCTTAGCGTGGTCGCGGCCGAGGTACTTTTTTTTTTTTTTTTTTTTTTTTTTNAATTTTTTTTTTNNT
TTTTTNNAAAAAAANTTTTNNTTTAAAAAAAAAAAAAAANTTTTNCNNNNAAAAANAAANNTNAA
AANNNNTTTNCCNGGNTTTNAAAAAAAANTNNTTTTTNNNNAAAAAAAAANTTTTTNGTTTNCCC
NNAAAAAAAAAAAAAANNNNNTTTTTTTTAAAACNNNTTNNTTTTTTTNNCCCCCANCNAAANTTT
NAAAAAANGGGNTTNCCAAAAAAAANNGNTTTTTAAAAATNGNNANANTTTTTTTNCCCNAANN
NCCNTTTTTTAANTTTTTTTAAAANNNANGNTNCNTNNTNNCNNNTTTNAAAAAAAAA

| | |
|---|---|
| Sequence 1288 | cMhvSA050h05a1 |

CCCTTTCGAGCGGCCGCCCGGGCAGGTACCATAGATCACTGGTAGGGGAAACAAAAGCAAAAGCA
AAACAAAACAAAAACAATAGATCCTGATGACACAGGTCTATTTATACAAACGATTGAAGCAAAAA
TCAATTGTAACTGTATCAGTTTATGCAGGGAGAAATGACAATTCTATTGTCATGTGGACTAGGACA
ATATTGGTGACAGGATGGGGTTTGGAAAGCTTCAAAATAATTGGGTGTTATGTTTAAACAGCTCAT
AGGTGCCCCCATTTACCACATACCCGTATTGGGGCCCGCCAATTTATTTTTCTTTCCAGGTTTTCTG
GTTGCCAAAAAATGCCTGGAATTTCCAACCCAACCCCCCTTCACCAATTATTTGGTACCCTCGGGC
CCGCGACCCACCGCCTAAGGGGCCGAAATTTCNCAGCCACACCTTGGGCGGCCCGTTACTTANGTG
GATCCGAGCTCGGTACCCAANCTTTGGGCGTTAATTCCATGGTCNATTAAGCCTNGNTTTCCCTTGT
GGTGGAAAAATTGGTTATTCCCGCTCACCAAATTTTCCCCACCACCAAACATTACCGAAGCCCGGG
AAAAGCCATTAAAAAGGNTGTTAAAAAGGCCCTGGGGGGTG

| | |
|---|---|
| Sequence 1289 | cMhvSA057b06a1 |

CCCTTTCGAGCGGCCGCCCGGGCAGGTACCTTTCTTTCCAGGCCATGGCAAAAAAATCCAATTATG
CCCGTCTTGAGTCTGTGGTCTTGCTTCTTATGTAGTATTTCCTTTGTGAGCTGAAGATTAATGCATG
GATTCACCTCCTTCAGCACATTTCATTTCAATTGTGAAGAAAAGATTCCAGGCACTGAATGTAAAA
TTGAACATGACATTTTGACATTCCTTCTTCTGAGAGCTGGGTTGGTCTTAGTTGCTGTGAGGCTCTA
GACACCGACCATACAGGGCGTGGGGCTGCTCCTGGACATGAACATCCTCCGAAGTTCTCCCCAGTC
CACTTTACCCCCTCTGGGCTCACCTCTGAATGTCCCCGCGTACCTNGGCCGNGACCACGCTAAGGG

| | |
|---|---|
| Sequence 1290 | cMhvSA010h05a3 |

CCCTTTCGAGCGGCCGCCCGGGCAGGTACGCGGGACATTCAGAGGTGAGCCCAGAGGGGGTAAAG
TGGACTGGGGAGGACTTCNGAGGATGTTCATGTCCAGGAGCAGCCCCACGCCCTGTATGGTCGGT
GTCTAGAGCCTCACAGCAACTAAGACCAACCCAGCTCTCAGAAGAAGGAATGTCAAAATGTCATG
TTCAATTTTACATTCAGTGCCTGGAATCTTTTCTTCACAATTGAAATGAAATGTGCTGAAGGAGGT
GAATCCATGCATTAATCTTCAGCTCACAAAGGAAATACTACATAAGAAGCAAGACCACAGACTCA
AGACGGACATAATTGGATTTTTTTTGCCATGGCCTGGAAAGAAAGGTACCTCGGCCGCGACCACGC
TAAGGGCGAAT

| | |
|---|---|
| Sequence 1291 | cMhvSA037a03a3 |

CCCTTTCGAGCGGCCGCCCGGGCAGGTACTTTAAGAAGTAATGCCCTTGAGTTAGAAAATCATCAT
TTTAAAATCTCTGATGATATAATGGATTTAGGCAATAATCATCAAAAAACTAAGTTAAGACTACAA
CCTGTCAACCAAATACCATGTGTAGACCTTGTTTGGATATTGACTTAAGCAAATAACCCTACAAAG
ACACTTTTACAATCAAGAAAAACTGAATGGGACTGCGCATGGTGGCTCATGCCTATAATCCCAGCA
CTTTGGGAGGCAGGTGAATTGCTTGAGCCCAGAAGTTTGAGACTAGCCTGGGCAACATGGTGAGA
CCCTGTCTCTAATATAATTTAAAAAAAAGAA

| | |
|---|---|
| Sequence 1292 | cMhvSA033h12a3 |

CCCTTAGCGTGGTCGCGGCCGAGGTACATGTTAAGGTTTGGTGAATGCATGCATTCACGGAACTAC
CACTCCAGTTGTGTTAGTTTCCCATGGCAGCTTTAACAAATTACTGCAAATTTCATGGCTTAAACGA
ACACACATTTATGCTTACACAGTTCTGGCAGCTAAATGACCAATGGGTTTCATTGGGACAAAATCA
AGGTGATGGCAGAGCCCTGCTTCTTTTGGGGGCTCTAGAGTCCATCTGCTTCCTTCCCTTCTCCAGC
ATCTGGAGGTCACCTCATTTATTGGCTTGGGTCCCTGAACTGCATCACCTTTTCTTTCTTGTGTCCAT
TGTTTTCTCATCTTCCTCCTCATCTGTCTGCAAATCTCCCTCTGCCTCCCTTTCAT

| | |
|---|---|
| Sequence 1293 | cMhvSA032e06a3 |

CCCTTTCGAGCGGCCGCCCGGGCAGGTACTTTTTTATAGAAGCCCAACTGGACTGACAGATGTCAA
GGGGTTGGGGGATCCTCAGTAGGCTAACCTAGCAGAGTTCTTGCTAAAACTGGGCTAGACAGGCC
ACAGACAAGATAGCCAAAATCAAAGCCTAGTTGAGAAGGGAATTCAGAGGAGCATGACTAAAAT
TTGGTCAAGGGGAGAGTCTTTGTCACCCCAGCACCTAGCACAAGTGGTTGGTACCTCGGCCGCGAC
CACGCTAAGGG

| | |
|---|---|
| Sequence 1294 | cMhvSA010h01a3 |

CCCTTAGCGTGGTCGCGGCCGAGGTACCTTCACTCTCCACCAAGCACCTGTTATCGGAAAACGTCC
AAACACTTTACATGTCTCTTGTGTGTTTTCATCACAAATAGAAACTAAAAAAAAACAAACAAAAAC
CCACAAAAGTTAACTCTGGAGATTATTCANAAACCGTTTCCTCAAAGTTTTATCAAACTTACCACT
ATCTTTAATCTCCCTACAGCACTCTCTAAAGATGTCTGGTAGGGTGCCTGTAACACTGCATTCTGCC
TACCTCTTTTTCTGTCTCCCTCCACTACACTGTAAATACTAAAACAGGACACTGTTTCGTTTGTCTTT
GTATTCCAAAACGCAAGCACAGTACCTGCCCGGGCGGCCGCTCGAAAGGGCGA

| | |
|---|---|
| Sequence 1295 | cMhvSA010g09a3 |

CCCTTAGCGTGGTCGCGGCCGAGGTACAGATTATTTCATAGCCCAGGTATTAAGCCTCGTGCCCAT
TAGGTGTTTTTACTGATCCTCTCCCTCCTTCCATGCTCCACCCTCCAAAAGGCCCCAGTGCGTGTTG
TTGCCCTCTATGTGTCCGTGTGTTTTCATCATTTAACTCCCACTTATAAGTGAAAACATGTTAAGTA
TTTCATGTTAGTTTGCTCAGGATAATGGCTTCCAACTCCATCCATGTCCCTGCAAAGGACATAATGT
CCGTTCTTTTTTATTGGCCTAATTCTTAGGCAGTCTTTTCTGGAATTGTGACAGAAAAGGTTCAAAG
CAGTTATTTTTTTTCATATTATATCCATAGTTGTGTTTTTA

| | |
|---|---|
| Sequence 1296 | cMhvSA032b04a3 |

CCCTTAGCGTGGTCGCGGCCGAGGTACGCGGGTGGACTGGGGAGAACTTCGGAGGATGTTCATGT
CCAGGAGCAGCCCCACGCCCTGTATGGTCGGTGTCTAGAGCCTCACAGCAACTAAGACCAACCCA
GCTCTCAGAAGAAGGAATGTCAAAATGTCATGTTCAATTTTACATTCAGTGCCTGGAATCTTTTCTT
CACAATTGAAATGAAATGTGCTGAAGGAGGTGAATCCATGCATTAATCTTCAGCTCACAAAGGAA
ATACTACATAAGAAGCAAGACCACAGACTCAAGACGGACATAATTGGATTTTTTTTGCCATGGCCT
GGAAAGAAAGGTACCTGCCCGGGCGGCCGCTCGAAAGGG

| | |
|---|---|
| Sequence 1297 | cMhvSA018a09a3 |

CCCTTTCGAGCGGCCGCCCGGGCAGGTACCACCTATGAAGTATTCTGCCTAAAGATATTAAACCTG
AAGCTTATCAAATCTGTAAATCTGACTACGACTTGACTGAAAATTTAGTGGCAAAGGAATATAGTA
AATGACATCACAAGGATATAGCATCCAAACCCAGAAAGCGGATATTCTTTAGGATAAATGACCCA
GTTTCCTCAACAATGAAATGGCCTGGAATAGAAAAAAGAGGGAGAACTTAAAATAACATACCAAC
CAAATATAGCACATGGATCCTGTTTTAATATGGATTCAGAAATCCAATTCTGAAATGACATTTTTT
AAAAATCANGAGGCCGGGCGTGATGGCTCATGCCTGTAATCCCAGCACTTTGGGAGGCTGAGGTG
GGCGGATCA

| | |
|---|---|
| Sequence 1298 | cMhvSA002e01a3 |

CCCTTTCGAGCGGGCGCCCGGGCAGGTACAGTCCACTANCATGGAAGCTATGGGTGTGGGCATNT
AAAANTGCCCCGTAAGCAGGTGTGGCCAGGCTGGGGCCNTTGGAAAAGNCAACCAANTNAAGAN
TGCTNANATCANACCANCCCCATCTCAAGTGCAAGATTGCCCAGCCTCCANANATCATGTNTCAGA
GGATANCTCTGTCANAACNNAACCCAGGCACANTTCAANTNCTCTGCNGNNNGTAGTTAGACTTC
TTTTATTAAGCAANTCTCCTTTTTTTAAAAAGGGAACTCTCGGTCCTGNTCTNTGCTGGGCAATCT

| | |
|---|---|
| Sequence 1299 | cMhvSA032d10a3 |

CCCTTAGCGTGGTCGCGGCCGAGGTACGCGGGTGAGATGGCAAATATTTATTAATCATCCAACTGT
GTATCAGACACTAAGAATAAGCTGGGAGGCCATGGCAAGTGAGGTCACCACAGTCCCTGCCACAG
TGGAGGTTATGGTATACAGGTAAGGCAGGGAAGAGCACTGCAAAGGGTTTGCCCATTGCATCAGT
CATTTATTTATGCACATGTTGATTCAACAATTATTTCTATGCCAAGCTGTCTTCAAGGTGCTGGAGG
AAATGAAGCGTACCTGCCCGGGCGGCCGCTCGAAAGGG

| | |
|---|---|
| Sequence 1300 | cMhvSA003g11a4 |

CCCTTAGCGTGGTCGCGGCCGAGGTACCTGCCATCCAATACGGTCATTAGATTGGGTCATCTTGAT
TAGATTAGATTAGATTAGATTGTCAACAGATTGGGCCATCCTTACTTTATGATAGGCATCATTTTAG
TGTGTTACAATAGTAACAGTATGCAAAAGCAGCATTCAGGAGCCGAAAGATAGTCTGAAGTCATT
CAGAAGTGGTTTGAGGTTTCTGTTTTTTGGTGGTTTTTGTTTGTTTTTTTTTTCNCCTTAAGGGAGGA
TTTAATTNGCTCCCAACTGATTGNCNCTTAAATGAAAATTTAAA

| | |
|---|---|
| Sequence 1301 | cMhvSA054c01a1 |

NGGGGCCCTTAGCGTGGTCGCGGCCGAGGTACTTTATGTTTACTCTGTCAGGAAAGCGTCAGATGT
TTTTATTTCCAATTATAAGTTTTGTAATGCATCATGTATTTTGCTGACAGTCTTCAAGTTCTTGAAAT
AGTGAACAAATTAACAGCAGATATNGGNGTGAGAGAATTAGAAAACCAACTGGCAACTCATATGA
TAGAATTCAGATACAGGGATGGGTGGAATGGGCTCATTTATTTTATTTTCTCAGTCATACTTTGTAA
TTAACTTAGGCNAAAAAAAAAAAAAAAAAAAAAGTACCTGCCCCGGGC

| | |
|---|---|
| Sequence 1302 | cMhvSA002e03a4 |

CCCTTAGCGGCCGCCCGGGCAGGTACAGAGCTGGAGGCCCAAACAGCCAGCCAAATCTTGCTGTA
TTTTATCCACCATAGTATAATCCAGAGACTGTGGACCCCAAATTGGGATGCTTTTAAAATCCAAAG
TAGTTCTGTATACACATTTGAAGAAAAATGCTGTTGAAGAAATGTATCCATAAAACACTTCAGGTC
AAAAAGCAAAAGAATATCAAGAAAAAGTTTAAATAACATGATTCCTACTGGTTTTAGATCATAAT
TATCATCCTATATTATTTATATTCCGTATCACTGTTATCTTTCTCTGACAAATAATTCTGAAATACA
ATACATTTTAAAGTTATGCAGGATTTTAAAGACCTCGTCTTCAACAAATACAAGAAGTTTAATAAC
AAACTTTAAATAAATGCTCATT

| | |
|---|---|
| Sequence 1303 | cMhvSA054d07a1 |

GGGGGCCCTTAGCGTGGTCGCGGCCGAGGTACCTGGGACTACAGGCACACACTACCATGCCTGGC
TAACTTTTGTAGTTTCTGTAGAGACGGGTTTCACCATGTTGCCCAGACTGGTCTCAAACTCCTGTGC
TCAAGCAATTCTCCTGCCTCGGGCATGNNCAAGTGCTGGGATTACAGGCTTGAGCCACCACACTCA
GCCATTAGGCATTTCTTTTTGTTCCAGAGGTCTGTGAAAAACTATGGAGACATGAAGGGCAGTGAG
CCGAGAAATCGTGGCGCCTTCTAACCTACAGGATAAGGGCGTATAATCAGACTTAGTTA

| | |
|---|---|
| Sequence 1304 | cMhvSA037h01a3 |

TCTAGATGCATGCNCCAGCNGNCNGATGGATNTCGTGCATAATTCGACCTTAGCNTGGTCGCGGCC
GAGGTACGCGGGGTCAAAGCCACTGTTTTTATAATCTACTCCTTATATAAAACATTAAGTGAGGCC
AGGTGCAGTGGCCCATTTCTGTAAACCCAGCACTTTGGAAGGCCAGTGCAGGTGGATCACTTGAGT
CCAGGAGTTTGAGGCCTGCCTGGCCAACATGGCGATACCCTGTCTCTACTAAAAATACAAAAATTA
GCTGGGTGTGGTGGTGCATGCCTGTAGTCCCAGCTACTCAGGATGCTGAGACATCGCTTGAACCTN
GGACGTGGAGATTGCAATGAGCTGANATCGAGACACTGCACTGCAGNCTGGGTAACAGAGTGAGA
CTTCTTCCCAAAAAAAAAAA

| | |
|---|---|
| Sequence 1305 | cMhvSA054a02a1 |

GGGGGGCCNTTAGCGTGGTCNCGGCCGAGGTACCCGGGTATAAGAATGAGACACAGTAGCTGCTT
TCATTGATTCTGTTCAACCGTTGATTGGAATTCCAAGCAAATGCAGCAAGACAAGAAAAAGAAGT
CACAACCGGAAGAGGTGGGGAGGAAGGCCGGGACAACAGCTCAGTAAAGCTGAGGTGCAAGGCT
GGGCACGGTGGCTCACACCTGGAATCCCAGCACTTTTGGAGGCCCGAGGTGGGAGGATCACCTGA
GGTGAAGACCAGCCTGGACAACAT

| | |
|---|---|
| Sequence 1306 | cMhvSA032g01a3 |

CCCTTAGCGTGGTCGCGGCCGAGGTACCACAGAGGCCAGCACAGCTTCTCGTGAAAGAGAGCTTC
TGTATTCTCAGTGGGATCCAGGCAAACAAGTAAATTCTGGCCCCACTCCCTCCACCACTCCTCTGG
GCTCACCTCCAGTCTGAAGAGATGCACTGGATCACAGGGAGATTAAATTCAAAGAAGACTGCAGG
CAAGGAGGGGCTCTGCAGCAGCTGTACCTGCCCGGGCGGCCGCTCAAAGGG

| | |
|---|---|
| Sequence 1307 | cMhvSA033g10a3 |

CCCTTAGCGTGGTCGCGGCCGAGGTACCAGTCATATTGGATTAGGGCTCATAATGTCATTTTAACT
TAATTGTCTGTCAAAAAATTCTGTCTTCAAATACAGTCACATTTCTAGGGTTTAGGATTTTAACATA
TGAATGCAGGGGGACAATTCAGTCCATAATACTGTGGTTATCACTTTTTGGTCTTAAGATGATTGC
TACAGCTCTACAACCCACATCTATTATAAAAACAAAAAGAAGAGAGAAATAAATTGAGAGAGGA
GAGTTCCTTGATCACTTTGCAGGACGTGCGACAGGGGGTGTTGCTCATCTGTTTGGCCACCACACA
TTCTCAGGCCCTTTGCAGGACAGGGAGCATGCTGACAGGCAGGTGCAGCAACCCAGGCGAGTGCC
TTGGGGCTCCAG

| | |
|---|---|
| Sequence 1308 | cMhvSA037c06a3 |

CCCTTTCGAGCGGCCGCCCGGGCAGGTACCACCTATGAAGTATTCTGCCTAAAGATATTAAACCTG
AAGCTTATCAAATCTGTAAATCTGACTACGACTTGACTGAAAATTTAGTGGCAAAGGAATATAGTA
AATGACATCACAAGGATATAGCATCCAAACCCAGAAAGCGGATATTCTTTAGGATAAATGACCCA
GTTTCCTCAACAATGAAATGGCCTGGAATAGAAAAAAGAGGGAGAACTTAAAATAACATACCAAC
CAAATATAGCACATGGATCCTGTTTTAATATGGATTCAGAAATCCAATTCTGAAATGACATTTTT

| | |
|---|---|
| Sequence 1309 | cMhvSA002a06a3 |

CCCTTAGCGTGGTCGCGGCCGAGGTACATGCCTGTAATCCCAGCTACTGGGGAGGCTGAGGCAGG
AGAATTGCTTGAACCTGGGAGGCANAGGTTTTAGTGAGCTGAGATCCCNCCATTGCACTCCATCCA
GCCTAGGTGACAGAGCGAGCGANACTCCATCTCAAAAAAGAGAAAGAAGAAGAAGAGAGCTCAA
CAATGCAGCCAGGGAAGATTTCCTGTAGGAGTCTTGAGACAGGAGAAAGAGAGATGGAAGAGAA
AGAAAGCGCATGCTGCCTCTGAAAAAATGGAGAGATCACCCCCGCG

| | |
|---|---|
| Sequence 1310 | cMhvSA058f01a1 |

ACTTTTTTTTTTTTTTTTTTTTTTTTTTTNGAGAGATGGGGTCTCACCGTGTTGCCCCAGCTGGTCTC
AAACTCCTAGGCTCAAGCAATTCTCGCACCTCAGTCTCCCAAAGTGCTGGGATTACAGGTGTGAGC
CACGATGGCCAGCCATAATGCGAAGTTTTAANAAGCTTTCAGGGANAAGGGANAGAGAATGCTCT
GGAAGCAGCCAAGAGAATCAATAGAGACATTCACCCATTTCCTGTCAGTGTTACAAGGAAGGTAG
AANAGGACAGAGCCATTGTTTGAGAAGCCTACAGGGCAAGCCAAG

| | |
|---|---|
| Sequence 1311 | cMhvSA032g06a3 |

CCCTTTCGAGCGGCCGCCCGGGCAGGTACGCGGGATGAACAAGCTCAGGAAAAATCTAAGAAGGC
CTTAATTTCTCACCTCTAGCTGACTTTCAGGCTACATAAACAGGAATTGAATGATAAGGTAGAAAT
GTGAACTCCCTGACTGAGTGTTGAAGGTATGCCCTACACATCCACAAAACCCTTGAGCAAAGACTA
AACTAAATAAGCAGAGACTTAAGTGGCCACACATAAAAAAGAATACAGACTGCAGAATGTGTTCC
CCCAAAAAATCACTAAGCAAAGAGCAGGAGTAACAATAAACAGCAACAATAAATCCTGCAGAAA
AGGAGATTCTGATTTTTAGAGTTGACACATAATATTATTTAAGACACTCAGTTTTCAACAAAAAAT
TATGAGGCATGCAAAAAAAA

| | |
|---|---|
| Sequence 1312 | cMhvSA031e05a4 |

CCCTTTCGAGCGGCCGCCCGGGCAGGTACATGAGATTAACTGATGTGTCTACGTGGTGCCAGTCTG
ACTAACAGTGGATGTGTGTGTGAGTGACCCTGCAATGTCATGATGTACCTCGGCCGCGACCACGCT
AAGGG

| | |
|---|---|
| Sequence 1313 | cMhvSA002d05a4 |

NGCCCTTAGCGTGGTCGCGGCCGAGGTACATGCCTGTAATCCCAGCTACTGGGGAGGCTGAGGCA
GGAGAATTGCTTGAACCTGGGAGGCAGAGGTTTTAGTGAGCTGAGATCCCGCCATTGCACTCCATC
CAGCCTAGGTGACAGAGCGAGCGAGACTCCATCTCAAAAAAGAGAAAGAAGAAGAAGAGAGCTC
AACAATGCACCAGGGAAGATTTCCTGTAGGAGTCTTGAGACAGGAGAAAGAGAGATGGAAGAGA
AAGAAAGCNCATGCTGCTCTGAAAAAATGGAGAGATCACCCCCGCGTCCTG

| | |
|---|---|
| Sequence 1314 | cMhvSA058g09a1 |

CCCTTTCGAGCGGCCGCCCGGGCAGGTACTTTTTTTTTTTCTTTCTTTCTTTTTTTTTTTGTATTTTTA
GTANAGACTAGGTTTTACCGCGTTAGCCAGGATGGTCTGGATTTCCTGACCTCGTGATCCGTCCGC
CTCGGCATCCCAAAGTGTTGGGATTACAGGCGTGAGCCACGGAGCCCGGCCATAGGCCTGTTTCTT
ATTCTATATTCCTGTTAATGTAAACCTCCTNAGATNGGAAGACAATCANTTTTACAGGGTAAGAAT
TGTTTTAATTATGTGGCAGCTTTTCTCCAAACATGAAGAGAAACATTAGAAATACGTTTAATAAAA
TCTCTATTATTTTGTTTTCTTTCAAGT

| | |
|---|---|
| Sequence 1315 | cMhvSA005f09a3 |

CCCTTTCGAGCGGCCGCCCGGGCAGGTACTGGGAATGACTGAGTAGTCACAAATTCAGAGAGCTG
CTGGGAGGTAGATGAGTTGGGGCTGGGAGGTGTCCATGGGATTTGGGGGCTTGAGGGTCACGGTC
ACCTCAAGACANCAAGATG

| | |
|---|---|
| Sequence 1316 | cMhvSA031a07a4 |

CCCTTTCGAGCGGCCGCCCGGGCAGGTACTTTTTTTTTTTTTTTTTNGGTTTTTTTTTTTTTTTTTTTT
TTTTTTTTTTTTTTTTTTTTTTTTTTTTTTTTTTTTTTTTTTTTTTTTTTTTTTTTTTTTTATTTTTTATTGT
TTTTTTTCCAAACCCANAAAGCGGATATTCTNTAGGATNANTNNTTTTTTTTTTTNAATAANAAAAT
GCCNNCNTANAAAAAAGAGGGANAACTTAAAATNCAACCAACCAAATATAGCACATGGATCCTGT
TTTAATATNGGAT

| | |
|---|---|
| Sequence 1317 | cMhvSA062c08a1 |

CCCTTAGCGTGGNCGCGGNCGAGGTACCTGCTGTCTTATGCATGTTTAACACAACAGCAACAATAA
TATAAGTAGTTAGCATATATTAAAGCNTTAACGAACACCAAGCATCGTTAAATATATTACATGTAT
TATTGCTTAATTTTCACAACATTACTAATGG

| | |
|---|---|
| Sequence 1318 | cMhvSA003e10a3 |

GTAGGAGGCAAAGTGATCTGCTTGAAAATATGNNTGAAAGATAATCAGCAAATAATTTCAAATCT
TGGAACTGTCATTATGAATTTACTGCCATTAGATTGTATTGAGGTCCCTGAAGTCATGGGATAACC
AGAAGGGGGAATTTGAAGATTCCATTTAATAAAAAGAAGTTGATACAAAGAAGCTAAGATATATA
ATAAAATTTTCATAGTTTGGAAGAGAACATGATGCTTCTGGTATTCCAATTACTGATTATACCTTTT
GTTCATAGNCTTTTTAAANCTGAGCTCTTTGGCCAATCCCATTTCAGCCCGCTTTGGTCTCATTAGG
TACCTGCCCGGGCGGCCGCTC

| | |
|---|---|
| Sequence 1319 | cMhvSA054c09a1 |

CCTTTCGAGCGGCCGCCCGGGCAGGTACTTTTTTTTTTTTTTTTTTTTTTTTTCAAAACTAGTGACTC
CTGTCACTCTNTTCCACTCTAAAAGGGCAANATGCAATGGCAAAAGGGCACATAATTCTGTTTCCT
TGAGTGTCTNTTAGTATTAANGNAGGCTCAGTTTNTAAATATTAAAATGACCCACAATAAGAGCTG
CAATGATTAAGTTTGTGACTTGTTATACCAATCAATGTATGACAAACTTANAAAAACTGTATATAA
TTTACAATGACAAGAGAGGAAAGAGGA

| | |
|---|---|
| Sequence 1320 | cMhvSA058d08a1 |

CCCTTAGCGTGGTCGCGGCCGAGGTACGCGGGGTTCAAAGTCTATTTTTATTCCTTGATATTGGACT
TTTATTTTTTTTTATTTGNGGATGGGGACATTGTGA

| | |
|---|---|
| Sequence 1321 | cMhvSA010c06a3 |

CCCTTAGCGTGGTCGCGGCCGAGGTACACAAACCCCTTTNCAAATGAGGACCGTGAAGAAAGGGC
CCAAAGTATCTGCACACACACAGAANATGCCCAGACAGCANCTAGTAACAGTTCTGGGTGCCACT
TACTATGATCCTGGANCAGCTGGGCTGCGATGGANACCCGGCNCCGCTCACCCGTGGAAATGCCC
CCCAAGCTGNANTTGCCAATCAGTCGGTCTGCCACATGGCTCAGACTCANNTCTNCCATGACNGTC
TNCACCTGCAGGAGACACAAATTACANGGAAGGCTGGGAGTCTCTGTGGCTGCTATTTCAATTCAT
GGGCTGGGGAGGACATGAAANANGCAGCANACCGCCCAAGAATC

| | |
|---|---|
| Sequence 1322 | cMhvSA002a11a4 |

CCCTTAGCGTGGTCGCGGCCGAGGTACTTTTTGCATTTTCAAATGACTTTGACTATTGCCAGAGTCA
TTATAGACCTGCCTATGATGTAGGAGTTTATTGTATCTAGTGGAAAACATACCTG

| | |
|---|---|
| Sequence 1323 | cMhvSA032g09a3 |

ACTTTTTTTTTTTTTTTTTTTTTTTTNGGNAAANTTTTTTNTTTNGGGGAAAAAAAANCNAAAAAAA
AANTTTTTNNNNAANTTTTNNNAAAAAAAAAAAANCNNCNAANNNNGGGGGGNTTTTNAAAAAA
ANTTTTNNNAAANCCCNNTTNCCNNNTTNNNCNAAAAANAAANNTTTNNNNTTNNANGGNNAAA
NNNNNTNGNTTTTTTAANGGGTTTTTGGGGGGTTCCCCCAAANCCCNAAAAAAAANAAAAATTNN
NNGNNGGGGGNNNAAAANCCCNTAAAAAANNCCCNAATTTTTNNNTTNGGNAAAANNCCCCANN
NNNNTTTTTGGGNAAAAANNTANCCCTTNGGNNNNNCNTNGGGNNAAAAAANGGCCCAAATANT
TTTTTTCNAANGGGGTTNAANNTTCCCAANTTTTTTTTGAAAANANNGGGGTNCCTTTTNGGGNNT
TGGNAAANNTTTNAAAAAANGGGGGGGGGGGNNTTNTNNNGNTGGGCNCCNTTTTAAAGGGGGA
AAAAAAANAGCNCCCCNCCCTTTTANNNTNANTTNGGGGAAAAAGNGGNCCCAANGGNTTTNTTT
NTNCCNNTNAAAAAATNTNTAAAAGGGCCNNGGGGGGTTTTT

| | |
|---|---|
| Sequence 1324 | cMhvSA004a11a3 |

CCCTTAGCGTGGTCGCGGCCGAGGTACTGCTGCATTTTTGTTTGTGTATTNANTCNTTNCCTTTGNT
TNCAAGTGAAATNTTTTGAAAACAGTCCTATTATGGCTCAAATAAGCAGAAATGGGGATTTTCTTA
GGCTAATTGANGAACATGGNGAGGGTGGCANGGACGACTGCTGACACANGGCACGCTGGCCTGG
AGAAGCAACAGCTGCTGGCNTGCGTGGACACCCTTTGCAGACGTGTCCCCTGCGGGGGATGATAA
TTCATCACCCTCCANCCCCCANCCTAGGGGCCTCTCACACAACCCCATCNTTTCACCACANAAGAA
CACANTGCCGATGTGCCNATGCTTCCAATCACCANGACCCAANGGTTGCCNACACCTTGGTCCAAN
ATGTGGGATCAAAATGGGGTGGATTATNTTNAGGGGGGCTNACTTCTAAATTTNAACAAGCCTGA
AACTTTCACTGGGGAAAATACTTTTTTAACCCCACTCTAANGNATTCCATTANANATGACATCCAT
TTTNAANTTANAAGACATGTTTTTACCTAAAAAATANATGAAAAANGCTTNGNNTTNAAAAATGG
GAAAAACCTATTGCTTTCCCCNAATNCCNNAANNNNNAATTTTTTTCCTTTAAANCNTTNNGCANN
AAANAAACTTTTNCTTTTNATTNANACNNCCTTTTTTTTAATTTT

| | |
|---|---|
| Sequence 1325 | cMhvSA004a10a3 |

CCCTTTCGAGCGGCCGCCCGGGCAGGTACNCGGGATAACTNTTCATGGGAATNAGATTTATNTCCC
ANATTTAAAAGCAAAAGCTCATAACAGCNNGGATTTCACTTAAAGGAAATACTTCTGAACATGTT
GTTAAAATATTGAAGAACTAAGGCCAAGATGTTCTGTTCATTATAAAAGTGGACTTCACTAGTTCC
AATGGTATATTATTTTCAGTGGATCAAATATATCTCATATGCTGGACTTTTAATGTCTGGACNCCAT
ATNTTNTGGAAGGGCATTNATTTANTNTTATTGNGGATATTTTCATTTTATNTTANCACACNAGAC
NATTACTNCAAGCANGAATCNCCCANAGAATGAGAAAANGCTCCTGGTCCTCAGAGGGCATNGNN
AANTAGGACAGGCCAAGACATNATTNTTTTGACTTGGGCTTT

| | |
|---|---|
| Sequence 1326 | cMhvSA002e06a4 |

TATCTGCAGAATTNTCCCTTNGCGGGCGCCCGGGCAGGTACAGACCTGGAGGCCCAAACAGCCAG
CCNAATCTTGCTGTATTTTATCCACCATAGTATAATCCAGAGACTGTGGACCCCAAATTGGGATGC
TTTTAAAATCCAAAGAAGTTCTGTATACACATTTGAAGANAAATGCTGTTGAAGAAATGTATNCAT
AAAACACTTCAGGTCAAAAAGCAAAANAATATCANGAAAAAGTTTAAA

| | |
|---|---|
| Sequence 1327 | cMhvSA003d03a3 |

NGAGGAATGATGAGCTCTCTAATTNTCTCCTACACAACATTTCTTATCAANGCCCTGGATCCCNAC
CTATGANAGCCTTCCAGGGATGCCCANGGTAAACCAAATGGGGCTGACCATNTGCCCATTGTTNG
GGGAGTGNAGTTGAAAANTAAAGGNAGCCCGGTCCCCTTTAACTTAANGGTGAGCCCCTTACAAT
NANGNGGGNACCNCAAANCTATTTCATANATCCCCCCCTNCCTTTTTTGGGTTCCTTTGGCGGAAT
TGNGGNCNANNAATGGAAAATGGGGCTTTTCGTGGGGATAAANACTTTTANAAATTNTTTTCAAC
CTTTTNNTTGGGNTTTNCAAGGGGGGAATTCCAAAAAGNCCCCCCCAAAATTNCTAAANANGNNA
AAATTTNNNAACCTNAAANCAGGGNAGNTCCANATGNNACCCCGGNCGATTNCCCCAACCAAAA
AAAAAAAATNGGCCCTTTCAAATNGGTTTGGCCTTGGAAACNCCCANNGGGNAANATAGGNAAA
GTTTNCNCTTAACCAANAAAAGCCCAAGNNCGGANAAAGGGGGNCNCCCTTCGGGAACTTTTTNN
TNAGGNNATTTTTANANATAAAACGGNTANTGGTTTTTAAAGGGGGCTTTNAACGNGGNAACCAA
AAGGGGCCTTTTTCAAANAAAAAAGNGTNTNCNANGGAACTTCCCCC

| | |
|---|---|
| Sequence 1328 | cMhvSA002f02a3 |

TCCGGGCTATGGTNGNNCNTNNAGCTTNTGCAGCCACCCCTNTGCTCTNTTTTCTGCCCTGGNCCCT
CTTCTCNNCTCCNAGAGCACCATGCCTTCCATACAAGGTGGNCANCCCTGTTGCTNCTNNAGNCTG
CACCCTTNCACACCNTTCTTTCTNATGACATTCCANCTGTCTGGAATATGGGCTTCCCACCCTCCCA
TTCACCTACCCTCTCACCTGGTGAGCTTACTGTNTNGNGCCCAGCTCANACGATATGGTTGAAGAA
TAGGTGTCACCTTCATCTGAGNACTCATAGCATATTTCTTATACCTGANAGTAAACAATTGCATGT
CATTATATGGCATTTAAGTNTGTCTCCTTAGATAGCCTCTAAGTCCCTTGANGGCAGGGACTATAT
CTTATTCATCTATTTGNCCTNAGNACTACTCAGTGCCCAGCCATAGTAGGTGTCCAATAAATATTTC
AATG

| | |
|---|---|
| Sequence 1329 | cMhvSA003d09a3 |

CCATTTGTCCCCANATGGTATAGNGTTAAAAAAAGGGGGTAANGCCNTTTAACTTGGGGTGTGNT
NCCCTTCCCCGNAATNTCCCAAGCGGTTTTAANTAAANTCGGTAAGCGNAAGGGGTTCTCGCCGGC
GGCCTTAAGGGGAAGGTTCCANATTAACANAAGCNTGTAATTCTCGGGGGCCTNTTAANNATNGG
GGGNCCCNGAAAAAAATAACTTTAAATTGGCCCTNTCTTGGNTTCGGTCTTTTGGGGGAAATTNAT
TTAATTGGCGGNAAGNGGGAATTCGGGGNGNGGGAAATCTTCAATTTTCGGCCTTTANGGNGNNA
ATTGANAAAGGGGNAATTNGGGGAATTTAANGGTTAAAAATTTAAGGNGNGNCCCAAAAGGGGG
AACCCCNCCCCCTTNCCCCTTAAAGATTTNTTCGNTTTNAAGGGGGGGGACCCCNGGAANTCCNGN
NGAAAAAAAAATTTTGGGTNGGNTTAAGGGCCCCCGAAAAANTTANNGGGAACCCCCCCCGCCNG
GTTTACCCCCTNTGNCCCCCCNGGGGGGCCGGGGNCCCCGGCNTTTCCNAAAAAAAANGGGGGGC
CCGGAA

| | |
|---|---|
| Sequence 1330 | cMhvSA002h08 |

ACCTATTAACATCACTCAGCTGCTGTGAAATAGGCTTACAGGCAACATGGAGTGTCAATTACCCAA
TGTTTAAAGTCGATCATACAGATTGGACTACAATCTCTATGGCTCATAAAGTCTTTAAAGGATTGA
CAGATGATTTATCTCATATGTAGACAATGATTCTCAGCAGTTAACTAGCGCAACTTGATAATATCA
ATTGCTTGAGAAAATCAGATAATTGCTTGAGAAAATTANGACATTGCTTGANGAAGNNCCGTNNT
NAANTAAATTNCTTCNNTGAAGGAACTNGTNAACCATCNNGGAAAGGACANCTNCNGGCTTGGGA
ATGGGGGACCTTGAATNATGCTGCTTCAAAAATTCTGGCAGCAATAACATGTTTAATTATGAAAAA
TAATGTTGGAAACAATTCAATTTTCTAGGCANAATNNTTCAAAAAAGATTTCGAGGCAGTCAATAA
AATCTGTTCCATTTAAAAGGATCACCTCCAATGCCANNGTACAAAGACTGCCCCAATCCNAACTTG
CGTNGTTTGGGGGGAACCTGCTTCATAAGGTCANGGGGCCCNNNTCTTGGGAACACAAATGCCCA
ATCCTTTCCNTTTT

| | |
|---|---|
| Sequence 1331 | cMhvSA003d05 |

CCCTTTCGAGCGGCCGCCCGGGCAGGTACAAGTATCTTAGGCTACTGGACCGGGCAGGCTTTACTG
AGGGGCTCCGTGCAGCTTGCTGGTGCAGCCGAGCAAGTGGGCCTGTAGCCGACTCTTAATCCAGGT
TGGTGCTATTCAAAGAGATCATCTTTCACCCGAGGGATTTCTGGGCACCTATTTTGCGGATCAGAA
AGTAGAGAAAGAAGGTAACTTTGCTGAAAGCTAGTCTGGGGAGTTAGTAGCTGATACAGATCAGC
ATTTCCTAACTATGAGATTTCATAATATTCTCTCTTGTCTCGATTCTGAGTCACTGGTGCCTGCTGT
GGTGGCATTGTTCATGAACATGTACCTCGGCCGCGACCACGCTAAGGG

| | |
|---|---|
| Sequence 1332 | cMhvSA009f06 |

CCCTTTCGAGCGGCCGCCCGGGCAGGTACAGAAGGGCCATGCTGTTATTACTCTTACACAAGGAG
GCAGCCCTCGAGCCACAGGGTCCAGCTGTTGGCTATAATAGCCTACCGGTCTCTGATGATCACCAT
GTTTCTGGAATTCAAGCCAGGAAGAAGCAGCAATCTGTCTTCTGGATTAAAACTGAAGATCAACCT
ACTTTCAACTTACTAAGAAAGGGGATCATGGACATTGAAGCATATCTTGAAAGAATTGGCTATAA
GAAGTCTAGGAACAAATTGGACTTGGAAACATTAACTGACATTCTTCAACACCAGATCCGAGCTGT
TCCCTTTGAGAACCTTAACATCCATTGTGGGGATGCCATGGACTTAGGCTTAGAGGCCATTTTTGA
TCAAGTTGTGAGAAGAAATCGGGGTGGATGGTGTCTCCAGGTCAATCATCTTC

| | |
|---|---|
| Sequence 1333 | cMhvSA011h04 |

CCCTTTCGAGCGGCCGCCCGGGCAGGTACCGCGGGAAATGTATACCGCTGGGAATCACTAATTTTC
CCATTCCTGGAGAGCCTGGNTTTCCACTTAACGCAATTTATGCCAAACCTGCAAACAAACAGGGAA
GATGAAGTGATGAGANCCTATTTACAACCAGCTAAGGCAAGAGACTGGGACTGAGNACTTTGGGA
ANAAAGTNTTCGACCCNTCANGAATGATAAAACCCAGCAAGNGGGTGGGACTTGCTTTGNGAAAG
AGACAGTTTNAATGGAACAAGAAGTTCTTTTTCAAGGACCCTTGGGNCCAGGTGGAAAANGGGGA
AGGCCCCCGGGGCCAAGGCCCACCCCGGNGNTTNTCCAGGAAACCCCCCTG

| | |
|---|---|
| Sequence 1334 | cMhvSA012f07 |

GGGGAGGCATTGAGGCAGCCAGCGCAGGGGCTTCTGCTGAGGGGGCAGGCGGAGCTTGAGGAAA
CCGCAGATAAGTTTTTTTCTCTTTGAAAGATAGAGATTAATACAACTACTTANNCNAATATAATCA
ATAGGTTACTAAGATATTGCTTAGCCGTTAAGTTTTTAACGTAATTTTAATAGCTTAAGATTTTAAG
AAGAAAATATGAAGACTTAGNAGAAGTNGCATGAGGAAGGAAAAGATGAAAGGTTTCTAAAACA
TGACCGGAGGTTTGGAGATGAAGCTTNTTCATGGGAGTAAAAAAATGTNTTNNAANNNGANANTT
GNGAGGANAGGGGCTACTAGAGCCCCCNNAATTNATNCCAAATTANAAAGGGNCCNGTGCTNTTT
ANNAATTAAAAATNNAAAGGGTGGACTTNAAACCNNGCTNTAAANGTNNTAAGTTTAAAAAAGTT
TGGGNGGGNGGNATTTAAAAAATAAAATNNTGGAAAGGGCGAATCCTTTTTAAAAAAANGAGAA
TTTAAACCCCCGA

| | |
|---|---|
| Sequence 1335 | cMhvSA016g03 |

CCCTTTCGAGCGGCCGCCCGGGCAGGTACACATGTCCAAGGTCAGGTCCTGGGTGGTNAAGGTAA
ATACAAATTGGAAGGGCACTGTGTGAGCCAAAATGAGTCANATTAGTCATGATTCATTTCCAGTTT
GGGTTTTGGGTGGTCTTGGAGAATGTTGNAAGCACTGCTTNATTGATAGGTTGATTGAGCCAGACT
TTACTCANCAGCCTGGAAAAGGAGAGATGGG

| | |
|---|---|
| Sequence 1336 | cMhvSA024c01 |

CCCTTTCGAGCGGCCGCCCGGGCAGGTACCCATATAAATCCCAAACCCCCAGCTCCAAAAGGAGA
TGAATAGAAGAGCAGAAGAATGCAGAGTGGCAAGGCAAAGAATGGGAGAAGAGAAGGAGCATCT
GAAAGTTGAGAGGAGTTGGGCTGGGGACGGTCCGAGAGGAGATTGGCCGCTGGATGGCCAAATTC
CAGGAGAAAAATAATCTCACTCCATCCCCCTTCCAGCTGCCCATCCACCCTGCTGAGAGCCACTTC
CATCACTCAATAAAACCCCCACATTCATCCTTTAAGTCTGTGCGACTTGACTTCCTGGATACCAAA
AAATTACCTGGGTCCCAAGAGGGCACCCGAGCTGGTTACACTTCTTCAGCTGTCTTCAGATGGCAA
ATCTAAAAGAGCACACTTGTACACACACCCACTTGGGCTTTTAAGGAAGTCACAGGCACCCACCCT
TAAGATCCTACCTTGGGGCTTGGAGCCCCAAGGCACTTCGCCTGGGGTTTGGTTGACCCTGCCCTN
TCAAGCAATGCCTCCCCTGTCCTGGCAAAAAGGGCCCTTGANNAAATTGTTGTNGGTNGGGCCCA
AACAAGATNGAGCCAAACNCCCCTTNTTCGGCACCGTTTCCTTGGCAAAAGTGNNTNAAAGGGAC
CTTTTTCCNCTTCTCCAAATNTAATTTCCCCNCCTTNCCTTTTTGGGTTTTNAA

| | |
|---|---|
| Sequence 1337 | cMhvSA032d03 |

CCCTTAGCGTGGTCGCGGCCGAGGTACGCGGGTGAGATACTCCCATCAGAATCCAAACAAAAGGA
CTATGAAGAAAATTCTTGGGATACTGAGAGTCTCTGTGAGACTGTTTCACAGAAGGATGTGTGTTT
ACCCAAGGCTGCGCATCAAAAAGAAATAGATAAAATAAATGGAAAATTAGAAGGGTCTCCTGTTA
AAGATGGTCTTCTGAAGGCTAACTGCGGAATGAAAGTTTCTATTCCAACTAAAGCCTTAGAATTGA
TGGACATGCAAACTTTCAAAGCAGAGCCTCCCGAGAAGCCATCTGCCTTCGAGCCTGCCATTGAAA
TGCAAAAGTCTGTTCCAAATAAAGCCTTGGAATTGAAGAATGAACAAACATTGAGAGCAGATGAG
ATACTCCCATCAGAATCCAAACAAAA

| | |
|---|---|
| Sequence 1338 | cMhvSA032f03 |

CCCTTTCGAGCGGCCGCCCGGGCAGGTACTTTTTTATAGAAGCCCAACTGGACTGACAGATGTCAA
GGGGTTGGGGGATCCTCAGTAGGCTAACCTAGCAGAGTTCTTGCTAAAACTGGGCTAGACAGGCC
ACAGACAAGATAGCCAAAATCAAAGCCTAGTTGAGAAGGGAATTCAGAGGAGCATGACTAAAAT
TTGGTCAAGGGGAGAGTCTTTGTCACCCCAGCACCTAGCACAAGTGGTTGGTACCTCGGCCGCGAC
CACGCTAAGGG

| | |
|---|---|
| Sequence 1339 | cMhvSA032h12 |

ACGCGGGGAGAGACAAAAACAGAAGAGGGGAAACATGTTTCCTACTGACGACAGGTGATTACAC
GTGTGCTTCTGATGGAGGGATCAGGAAAGGATATGAAAAATCCCGAAGCTTAAACAACATAGCGG
GCTTGACAGGCAATGCTCTGAGGCTCTCTCCAGTAACATCACCCTACAACTCTCCTTGTCCTCTGAG
GCGCTCTCGATCTCCCATCCCATCTATCTTGTAAACCAAACAACCAAACTGCATCAGTCGGCTAAA
TTGTATTAATTCAAGTGCTGTTTACCCCATAATGGAAATAATTAAATGTAGAGTTACTCCAGGCTC
CATTAATACAGTATAAATCTTGCATGATACTACAATTTGAA

| | |
|---|---|
| Sequence 1340 | cMhvSA033c09 |

CCCTTTCGAGCGGNCGCCCGGGCAGGTACCACTGTGCCTAGCTGAAACATCAGTTTCTGACTGAAG
TGGAGACTACAACAACTTTAGTGTTTCCCTTANAAGGATTACGGCCATGGGGAACTTGACTGAGTA
AACAATGCTATAAATAAAAAGCTCTTCCAAAACATTAACCATGGTAAGCATCATTATCCCCATAAA
ATGGTGGCATCCAGGTTAAAATGGCCCACCANGACCAAAAGTCTAAAATGGAAGATAGGAATCCA
GTCCGTTAAACTTTTTTCTGTATCTCCATCCGGGNGTGGGTCACCAAAGGGATTTACCAAATGCCTT
TCCTTTAGCATTTAAATTTCAATCCTGGGGAAAAAATTTTTAATCTCCCGTTGCCAATAATTCCCAG
TGGAGCTCTTCACCCAATACCTTATTTCCTTTTAATTTGGNGGGGGGGTCTGGCAACCGGGGGCCT
TTCCCAAAAGGANNCNAAGNAGNGGGATTAAANGNAGNAACCTTGGGTTTTTTT

| | |
|---|---|
| Sequence 1341 | cMhvSA043b04 |

CCCTTTCGAGCGGCCGCCCGGGCAGGTACCACAGACAGGCGCAAGAGGGAGGAAGAAACTATAA
ACCGAAAAAGAAACTGACAAACTTCTCTAATTGGGAATTTACATGCAGAGAGTGAGAGAAGATAC
ATCTCCCCATAAAAGGATTGAGAGGCTGTCAGATTCTCTGGCTGTGCTGTTTGGTGAAGGTCTTCC
CCTATAGAAAGCCAGTATGTAAAGATTGAGAGAGGTGGCTATTTTTCAAATGCAAAAATCACAAC
AAAAAATNACAAGGCACACAAAGAAACAGGGAAATCAGTCAAAGAAACAAAATAAATCTCCATT
AACTGACTCCGAAGAAACAGAGATCTATTAGTTACCTGAAAAAGAATTTATGATAATCTTAAAGA
AGCTCAATGTGTTTCAAGNAGAATACCAGATAGACAGCTTAAAATGGAAATCAGGCCAAACCAAA
GGCATTGAACAGGAATTGAGGGATATTGAACCCAAGNATTTNGGAAAACTTTTAAAAANAGGAAC
CCAACTTGGAAATTTCTTGGAGCCTGAANAAAAAACAACCTGGGTTTANGGAAAAAATTTNACTT
GNGGGGAAGAACCCANCCNAAGGNGGACTTTGNTCCAACCAGGGGAAAANAAATNCAGCCTTAN
CTNNAAAANGACCANAGTTCATTTTTGAAAAATTNTTTGNGNTNCNGGAGNTNACCCACCCCCCN
AAAAAAAAAAAAA

| | |
|---|---|
| Sequence 1342 | cMhvSA050c08 |

CCCTTTCGAGCGGCCGCCCGGGCAGGTACGCGGGGAGGCATTGAGGCAGCCAGCGCAGGGGCTTC
TGCTGAGGGGGCAGGCGGAGCTTGAGGAAACCCGCAGATAAGTTCTTTTCTCTTTGAAAGGATAG
AGATTAATACAACTACTTAAAAAATATAGTCAATAGGTTACTAAGATATTGCTTAGCGTTAAGTTT
TTTAACGCAATTTTTAATAAGCTTAAGATTTTTAAGAGTAAAATATTGNAATTACTTTAGAAAGGA
GTTAGCAATGGAGNGGAAAGGGAAAAAGGAATTANAAAAGGGTTTTTCTAAAAAACCATTGACC
GGGAAGGGTTTGAAGNATTGGAAAGGCNTTTCCTTTTCATTGGGAGGTTAAAAAAAAAACTGTTC
NTTTTTAAAAAAANGGNAAAAAATTTTGGANGNAGGAAAAAGGGAANTTNCCAAGGAAGCCCCC
CGGAAATTTTNATTANCCCCNAAATTACGAAAAGGGGGCCCAATTGGCCTT

| | |
|---|---|
| Sequence 1343 | cMhvSA050c10 |

CCCTTAGCGTGGTCGCGGCCGAGGTACTTTTTTTTTTTTTTTTTTTTTTTTTTTTTTTTTACNTCATTA
CTTTTTATTTTGAAAGATTTGTGAAACTNTTCACATCATGGNGAGAGTTTGTTTGATTAATAANAAN
CNNCTTTTTCATAGAAATGCTTTGGAGGTGAACNANTTCTNAGCCTNTGAGAATCCCGACCATCCC
ATTAACTTTGGAAGTTTCTCTTTGNTTAAATAGGAAGGAAACAACAGGGGGAGGGGTTGAAAAAA
AAAGGGAGGGAACCNTGCCTAAAAAACCTTNTTGACAATCATTCCCAAATGTTGAGGNAAAAGAA
ACAACCCCGGATTCACCCAAACNTCCCNCCTTTTTTTCTTATTTTTACCAACCTTTTCNTANAATTTT
CAACNTTCTTTTGNATT

| | |
|---|---|
| Sequence 1344 | cMhvSA052d04 |

GGTACGAAAGAGAGACAAAAGGGTTCTCTTGGAAACAAGAAGAGTGACTCCAGATGTGGCCTGA
ATAATTGCCATGTTAAGTTAATGCAAAAGATCAGAACAGGGCTACATTTGCACAGGCAGTTTCTCT
CCGGGCCGTAGTTTTCACTGATGATCACCTTTCACAGCATTTTCCCCAACCAAGCATTTCACTTAAG
NCTTCTCTATACCCAGCACCTCCCCCGGCACCCCCGGCAAGCCCCACTTATCACTTCCCGACTTCCA
ACGTGGGCATTCCCGTGGAGAATCTGGTCCACATTTAGGGCCGAAGCCAGGGAGAACACTTGGAG
AAGCAGCAGGGATGGGGTTTNGGAAAAAGAGCAATGCCTTTTGGGGAAACACCAGCTTTCCTGGG
GAATTTCNACATTGAGGCCAAGGTCCTTACAGAAGGAGCCAAAGAATGCACCCCCCAGGGATTTT
CTTTCNATTTTTTCTTAATTANATGTNGGGGAGGTGGCTTCNCATTTTTTCCCCCGGACANGNGGAA
ATTTTNCCCCTNGANNAAAACCGATTANCTTAGACCCCTTGGGGTTTTGGCCCCACCCTTTTGGTA
AACTTCTTTNCCTTTATCTTNCCTTNCTTTTTTTCA

| | |
|---|---|
| Sequence 1345 | cMhvSA056e12 |

GGTACTTTACCCTGCACAGATGCCCTCCTTGCCCCACTCAAGCTCCAACACCTGGAACTGAATAGT
CTTCCTGTATAGATACCCTCCCCACCCTACTTGGACTCTGGCATCTTTGTCTGGGTAGCTTTTTCCC
AAGGTGGTAGGTTGCTTGATAGGTGCTTAGTAAATATCATATTTGATTAACTTTTTGTAGCCTCCTC
TTTAGTCTAGAAATTCTAGATCCCAAATAGAAGGTAAGATATGGTATATTCTGGACTTTTAGTTTTC
TATATCTCCTTTTCAAATACAAGACCTAGGGTGACAGACAAAAAAATATTGTGATCAAAGTATATA
GCATTTNCTTTCATG

| | |
|---|---|
| Sequence 1346 | cMhvSA002e07 |

CCCTTTCNAGCGGCCCGCCCNGNCNGGNACTTNNNNNNCACNNNCNNTATGGNCTNAGAAANGNG
GGCCCCATTTTNCACCCTAGCTACAAANGGGTGAGTTTGAAAANTATGTNAGANNANCTGGANGC
TCAGGGGNCNGATNCTCTNNTGGATAANACCATTCAAAGCCAANGGTCNNGANGCCNACGAGCCC
ATACTGNTNATAAATNNNNNCCAAAAANTGNCCNTNTTNTTTGGGGNCCGCNGAGGANATNNNGC
CNTGGGGCTAACCAAAATATTAAATAGCGGTCCTTGAANGTGTACNGNGCCCNGGCGGNCGNTCC
AAAGGGCGAATTCCAACACACTTTTAAAAANTACTACCCGGATCCNNNCTCTTTTCAATNTTGGCC
TAATNANNGTTTTAGNNGTNTAANGAAGGANAANTTTTTTTNCCGGGNCTNAAAANTNGNNGGGN
TTTNNNGNAAAAAAANANTTTTTTTCCNANANANNNTTTNNTNTTNGGNNCCNCCCCAAAAAAAA
AAAAAAGGCCCNGTTTTCCCCTTGGGGGGGGGNTCNNAAAAAATCTTTCNANTTTTTTTTTTTTTTN
GAAATNAAGGNTNNNNCCCCNGNAAACCCTTNAAAAAANGGGGTTTTTTAAAAAAANCCNCCGN
GGGGGAANTNNTTTAAAATTTTAAAAAACCTTTTTAANGGGGGNGTTTT

| | |
|---|---|
| Sequence 1347 | cMhvSA003c08 |

CCCTTNGGCCGGCCGGGCAGGTACATCNGTCCCTTGACCATTACACCCACGGNGGNCCTAATTGGC
CTNTCTGGTTTCCAGGCATNNGGGGANAGAGCCTGGAAACNCTGGGGCATTGCCATGCTGNNGTG
GAAACATATCCCCTCATCCCACCACTGNGGGGCATNCTGTAGGAACATTNNCAGACTNCATGAGA
TAATGNTTNNNAATAATAACAATGGNCTGACAGTTNNAACTTTATTTGC

| | |
|---|---|
| Sequence 1348 | cMhvSA003d08 |

CCAGGTTACTTGAAATNATATGGGTATCAAAGTANCCATTGGAGAAACTTGTGGNAATGTCTNTGG
TGGNATCTGTAAAAAGAAGATTTCANCTTAGCTCATNGGGCNNGGGGCANGANGAANTANAGGA
NANTGNAATNNGGGACAGAAAAATTACNGCCTGGACTTACCAGATTGNGCTTGGCATTTTNNCGN
CTNAGNAGGGGCCCCTTNAANAATAATTTTNCTTNTCCTGGTGATTACAAGGGGNAAAAANAATT
TNGTACANAATAAGNGGAAGGGCCATAAAAATTNGGCNAANGCNTTGNCCACAAGAGGAACCAT
TTATATTANAACAANTTNANCCAGGTAAGGNTGNAAGAAATTTTGGAATNTTCCTTANAANAAAN
TTGGGTTTNTTTNATTGGGTNAAAANAAAATTANTTTTTAAAATTTTTTTTATTAACCNTCCATTTT
GGAGGTTANTTNACCAAAATAANAGTGGNANATTAATNTNCCTTCCTTTTAAAANAAATTNCCCAC
ANTTATNATTCANATTNTACTTTTTTTCCCAAANTNTTCACCACAAAAAAANTNGGGAANGTTAAN
ANAAAAAATTANTNATTGGTCTCCCTTTTTTTTNTANGGGGAATAANNAAATTGNNTCCAGGGGAN
ANTTAAANAATTGGAATTAAAATTACCACTTNCAATTANTTTC

| | |
|---|---|
| Sequence 1349 | cMhvSA004a08 |

CCCTTTCGAGCGGCCGCCCGGGCAGGTACTNTTTTTTTTTTTTTTNNCCCNCCNNANTNNCANTTNN
TTNGGNGNACNGTNAANGGNCGNGCCAAAATNAAGAAAGCACCCTTTTTTCCAANNAAAGANNN
CCATTAAAGNCCCACGTCCATNNNCNNGGGGTACTTGGNTAAAAAATAAACAAANNTTTTAACTG
GGNTTGGAAAAAAAAAAAACNNAGGGTCCCCCAGGNAAAAGGCAATNTTTTTTTTTTNTCNAAAA
AAAGCGNANGTNCCCNTAAGTTTGCCNATAAAAAAGGNAGGNCCCCNGNAAGGGNNNCCTTGNG
NGGNAAAAAACCCTNTTTTTTTAACCCTACGGGTNAAANAAANTTCANGAAATTTANNTGNNGNA
AACATGGNCTTNGNNAAAACGGGCCGGGGAAAAAAGGGG

| | |
|---|---|
| Sequence 1350 | cMhvSA004b05 |

ANAATTCGCCCTTTCGAGCGGCCGCCCGGGCAGGTACANAAAANNATGGCCTGCCAAANCTTTTTT
TTTNTTNTTCCAGGAAAAACAGGCCACAAATGAATGGTGTATTACAGATTGTACACACATGAAGA
GAAGGTAATANCGCACTGCNAAGCAGNCCGGCTCTGGGGAAGAACTTCACGGANCCCCTTCTTAG
AGCAGGGAGGGGGCTTTNTCAAANAAATGTTGAGGCTTTCTGCTGCCTNGNTCTGCCCCAGGCCCC
CCTCCAGGGTACCTCGGCCGTAACCACACTANGGGCGAANTCCNGCACACNGGCGGNCNNANCNA
CGGNATCNGATCNTGGGCCNNGACNTGNGNGAAAAAANGGCNNNANNTCCTTTCNTGGCACCAA
CTATGATGTCTTTGANAAAGATATGCTTGGGGGCCTGGGAAATTGA

| | |
|---|---|
| Sequence 1351 | cMhvSA004b11 |

CCCTTNCCAGCGGCCGCCCNGNCNGGNACTCGATNAAAAGTTTGGAGGCNTGNCACAANNNTGGA
AANAATNTAATGNTGNATTGACTNTNCAGGGTTCTATTAATGANAACACANTCNAACNANNTTTT
GATNTATTANNACAGATGTATAANNCCTATNATTTTTNAAATNAGNATCCACCTGACATTTATCTC
TCATTCCATCAGC

| | |
|---|---|
| Sequence 1352 | cMhvSA004h08 |

CCCTTAGCNTGGNCNCGGCCGACGTACTNTNTNTTTTTTTNTNNTGNTAAAGNAAGGGGNNCCNNC
CTATAAACCCNNGNNNGAATCNNNGNGGCCACCTTNGNGGNCNNNANGCTCCTANCCCNAGGGA
ANAANCCAATGTTCNGGACTNNCCCCCCCNAAAAAGGGGGNNTAANGGNCCCCCCNNCCTTCCNG
GNNNANTTNNNATTTTTTNACAAAAAANGGGNTNCCCCATTNGGCCGGGNNGGANNTAAAANNN
NAAANAAAANTTCCCCCCCCGGGANGNCCNNNAAAANGGTGGGGNNTAANAGCTGNTNNCCCNC
CCTNCCGGGGGGANNCAAAANNNCCTTTTTAGGGANGGGGCCTTCNTTTGGNCCNAANNTNTNTT
TTTGNAAAAGGCCCCTAAAATTTTTCCCANAAANCTTTTTT

| | |
|---|---|
| Sequence 1353 | cMhvSA005c05 |

NCCCTTAGCGTGGTCGCGGCCGAGGTACTACAGAGGACATAGCAGTATTAAGGGATAATGAAGTC
ACAGCTTCAGAGCCTCCATCCTTTCTTTAGCAAGTTAGCTCTACTTGTATCTGTTCTGTTTTATATAA
TATGGNTGCATCTAACTGTTTTTAAAAAAAGTTCTGTTCTTCAAAAAAATTTTAAGCTATGAAAAT
CACTGATTAAGTCAAACCCTCATTTTACAAAAGAGGCAACACAAACTCAGAGCACTTATGCCTCAC
CATAGGTCACAAAGCCAAGTANCTCCAGGCCAGAAAATGGGCTTTANGTCTTCCCGTCTGAGACT
GGCATTTG

| | |
|---|---|
| Sequence 1354 | cMhvSA008e08 |

CCCTTAGCGTGGTCGCGGCCGAGGTACCGCCCANTCTTTTACATGGTGATGGGANACACNCTTNAN
GCANACTTNANGTCTANTTNTGCCNNCATAANTNTNNCTNAACNGATTTACGGNACNCTCCNCCA
GATTTCATAATT

| | |
|---|---|
| Sequence 1355 | cMhvSA009c07 |

CCCTTAGCGTGGTCGCGGCCGAGGTACTTTTTTTTTTTTTTTTNNNANTTNNCCNTTGGCCCCCCNG
NNGGNNGNCNANGGGNGNANNCCAACCTANNGGNNANATTTNCCCCCCGGGNNAAAAAAANTNC
CCCCCCCCAANCCCCCNNNNANGGGGGNNTAANGGGNCCCCCCCCCCCCCCNAAAAAANTTTTGN
NTTTTNAAAAAAAANGGGGNTTTNCCNNNNNGGNCNNGGGGGGTTTAAAACCCNGNCCCNAGNN
NAACCCCCCNNCCNAACCNTGGGNTTTNNNTTNTTNNNNANTTTTNGGGAACCCCCNNGGGNTNN
NCNAANANTTAANNGGGTNNGGGGCCNAAAAANNGNCCCCNGGGGNNNCCCNANGGGCCTTTTA
AANGGNCCNNCCAAATTTTTTNGNAAACCCTCTTTTNNAACCCAAAANGGNCNTNAAATTAANGG
GGGNGGGGGNNCCCCAANCNTAAGANGGGGGAAAGNGNCCCTTTTACCCCNCTTTNTAAAATTTT
NTTTNAACCNGGGGCNAAAAAGNTTTTTNNNNAANGGGNANCCAAATTTTTNTNTTTTTTNNANA
AAANTTTTCCCNNGAAAAAAAAAAAAANACGNNGGGGAAAANACCCGGNGTTTAGAAAAAAAAA
AAAA

| | |
|---|---|
| Sequence 1356 | cMhvSA010b11 |

CCCTTAGCGTGGTCGCGGCCGAGGTACTTTTTTTTTGTTTTTTTTTTTTTTAAANCNTNGNAAAATNT
NTTTTTNNTNNCCCNGGANNAAACCCACCNTNTNTTAGGGNNNAAATAAANTAAANNCNNTCCNG
TTTTTNNTTTTAATCCCTTTAAAAAAGGGAANCAAAAAAAA

| | |
|---|---|
| Sequence 1357 | cMhvSA010f12 |

CCCTTTCGAGCGGCCGCCCGGGCAGGTACTTTTTTTTTTTTTTTTTTTTTTTNNNANGNCCNNAAGG
GGNAAANNNTTTTTTAAAAANCNNNTTTTNCCAAAATTTTGGNCNNAANTTCCCTTTTAANTTTNC
NNNNTNGGNAAAANGGGNTTTTNNCCNNNNAANCCTAANNTNAAGGGNCNAAATTTNTTTTNAAA
NTTNAAAAAANCCNNCNAAAAANCTTTAAAANNTTTCCCCNGGGGGCNTTTTTTCCNTNCCCCAA
AATTNTAAAAAGGGCCTNNTTTTTAAAGGAANTTTNAAAAAAGGGGGGGNCCNGATTTTTTTTNTT
TT

| | |
|---|---|
| Sequence 1358 | cMhvSA010g01 |

NGTACTGATNTNGNCTGNCNNANAGGAATGTATAATNTNAGGNCGNCCCTTATNANGCATGATGC
TTTAAANNCNTNNTACAAGTAACTTTTTAAAACNTNCCCTGAAACAANATGAGGGGACCCATT

| | |
|---|---|
| Sequence 1359 | cMhvSA012d02 |

CCCTTAGCGTGGTCGCGGCCGAGGTACTTTCCCACATTCCGGGTTGAAGAGAGCCTTTCAAAAGCA
TCAAAGATGGTTCCACAATGTTCACATGTCCACTCCTTTTATTCTCTTCTTTCGGCATGAAGTCACT
TGAGAAGGATGAATTTGTTTGGAGGAATGCTACTTTCAAATCCTATATGGGGAGGTATGATTTTTN
ATTTTTTCTAATTCTTTTCTCTTANATTAANTTTTTATCCAAAACTTTGTGAAAATGAATGGGAGCC
TAAAAAATACCTTGAAATTCTTGGGAATTCATTTCANGTCCACCCATTGGATNGNTTTTTCCCTAAA
TGGGGGGGCNTTCCCCCNAGGGGAGGCNATTTCCTTTTAATTNCNCTGAATTTATTGGAGGGGTTT
TTTTGGGTTAANCNCCAANGAAAGGGGNCTTAAAAAAACCCCAAAATTTGCCTNGGGTGGNCTTT
TTTGGCCTTANACCCTTCGGGATGGGCCCCNNGGGAANNANGGGNTTCAACCCGGGTTTTTTTANA
AAAAAANGTNGNAAAATGTCCNATTTTCCANGGGGNANTANTTTTTTGG

| | |
|---|---|
| Sequence 1360 | cMhvSA012e08 |

CGCGGGGAGGCATTGAGGCAGTCAGCGCAGGGGCTTNTGCTGAGGGGGCAGGCGGAGCTTGAGG
AAACCGCANATAANTTTTTTTCTCTTTGAAAGATAGAGATTAATACAACTACTTNCNAAAATATAG
NCAATAGGTTACTAAGATATTGCTTAGCGTTAAGTTTTTAACGTNATTTTAATAGCTTAAGATTTTA
AGAGAAAATATGAACACTTANAAAAGTAGCANTGAGGAAGGAAAAGATAAAAGGTTTCTAAAAA
CATGGACCGGAGGNTTGAAGATGAAANCTTCTTCATGGGAGTTAAAAAAATGTATTTNAAAAGAA
AAATNTGANAGAAAGGGGCTNCCAGGAGCCCCCGGAATTAAATACCAAATAANGAAGGGGCNAA
TGGCTTTTAAGATTAAAAATGGNAGGGTGACTCAAAACAGCTTAAAAGTTTT

| | |
|---|---|
| Sequence 1361 | cMhvSA012e08 |

CGCGGGGAGGCATTGAGGCAGTCAGCGCAGGGGCTTNTGCTGAGGGGGCAGGCGGAGCTTGAGG
AAACCGCANATAANTTTTTTTCTCTTTGAAAGATAGAGATTAATACAACTACTTNCNAAAATATAG
NCAATAGGTTACTAAGATATTGCTTAGCGTTAAGTTTTTAACGTNATTTTAATAGCTTAAGATTTTA
AGAGAAAATATGAACACTTANAAAAGTAGCANTGAGGAAGGAAAAGATAAAAGGTTTCTAAAAA
CATGGACCGGAGGNTTGAAGATGAAANCTTCTTCATGGGAGTTAAAAAAATGTATTTNAAAAGAA
AAATNTGANAGAAAGGGGCTNCCAGGAGCCCCCGGAATTAAATACCAAATAANGAAGGGGCNAA
TGGCTTTTAAGATTAAAAATGGNAGGGTGACTCAAAACAGCTTAAAAGTTTT

| | |
|---|---|
| Sequence 1362 | cMhvSA015a06 |

AGCGGCCGCCAGNGNGANGNNNTTCGGGGGAATNAAACCCAGCGCGGCCGCGGCCGAGGGACAG
NGNNNAAAAAGTGTACNGAAACAANAAAGCAGNCAANCAGNNAAACCCCAGAGAANNCNGCAG
AAAAAANNNATNNNCTAGNNACGGGNAGGNAACCNCACNAAAATGTGGACCGCNTNTTACCCNG
AAAGGAAAAAAACCCCCCGCANACAACCNCNACANNNCAGNCACGCAACCACAGGGCAAAGAGA
AANNAAGCTCCACNNNNAAAANANCNGAAGCAGGGGGGNAAAAGGCCCGAGNGGNCANNNNNC
NGAAANNCAGAGAAGCAANCAAAGGGCAGAANNNNGGCANNNNNCCNNANAGAAGCAGGGGGG
AGCNAAGGAGNGGCCANCAGNGAGGCACCNNGCCCCAACAGGAACCCNGGGGNAAGANAANGG
GAGGGACCGCAGCCNNGAAANANNNNCACCCCNNAAGCCACCGGGGGCNGG

| | |
|---|---|
| Sequence 1363 | cMhvSA015b10 |

CCCTTTCGAGCGGCCGCCCGGGCAGGTACAGTCAGGGTTTTGTCATGTTGTTTAGGCTGGTTTTGA
ACCCCTGGACTCAAGCAATCCACCCACCTTGGCTTCCCAAAGTGCTGGGATTATAGGCATGAGCCA
CTGCACCCAGCCAATTCTCCAAATCTCACAGCCAAACTGCAACTAAATTCCATCTCAAACAAATAT
TCAAATGCAGAAGACTCACCCATCTAATCAAGGCAGTTTTAATATTTAGGGGAAAAAAAATGCCT
GGATAAAACTGTAAAACCAAGCATGATAGAAAGAGATACTTTTAGGAATGGGGGAGGGGATGAC
AAAAATAAAACGAGAAGGTAGATAAGAATGGAA

| | |
|---|---|
| Sequence 1364 | cMhvSA016a04 |

CCCTTAGCGTGGTCGCGGCCGAGGTACTTTTTTTCTTTCTTTTTTCTTTTTTTTTTTTTTTAACAGGAA
TCAAGTAAAAACCACAGAACCTCTATATTTATATTTGAGTCTGAATCAAACATTTTCACCTGGAAG
AATTTTTTCCAAAGGAGGGGAAAACAACTGTTTCTGANTGCCTTTATTTTAGGTTAATTTTTTCAAA
AGATTATCTCTGACACCTTTGCATTAAGTATCTAATGTATTAGTGGGACTCCATGGTTTGCATTTAT
TTCTTCAATTTGCTAAAAAAAAAAAAAAGTCTACTAAAATTTCAATTTTTGAAAAGCAATTAATTA
GAATNTNTTAGATAAAGCAAAATGTAATAAACTCTTCACTTATTTTTTGGATGGAGGTCCTACTGG
TNATAAGATTTCAAGTTAAATTTTCCTAAATTGCCCTTTTTTAA

| | |
|---|---|
| Sequence 1365 | cMhvSA016b01 |

CCCTTTCGAGCGGCCGNCCGGGCAGGTACNCNGGGTGTGACCCGAGCGGTAACATCCAGAAAGGA
TTTCCNNCANANACNGCGCNGNTNNNNAGCTGCAGNTTGCCCCACCCTGATCCAGTCTCCCTCATT
TACAGCCTGGAAATTGAT

| | |
|---|---|
| Sequence 1366 | cMhvSA016d11 |

CGGGGAGGCATTGAGGCAGCCAGCGCAGGGGCTTCTGCTGAGGGGGCAGGCGGAGCTTGAGGAA
ACCGCAGATAAGTTTTTTTCTCTTTGAAAGATAGAGATTAATACAACTACTTAAAAAATATAGTCA
ATAGGTTACTAAAGATATTGCTTAGCGTTAAGTTTTTAACCGTAATTTTAATAGCTTAAGATTTTAA
GGAGAAAATNTGAAAGACTTTATAAGAGTAGCANTGAGGGAAGGGNAAAGGATAAAAAGGTTTN
TAAAAACATGAACGGGAGGGTTGAGGANGAAAGCCTTCTTCATGGGAGTNAAAAAAAAATGTTNT
TTNAAAAA

| | |
|---|---|
| Sequence 1367 | cMhvSA016d11 |

CGGGGAGGCATTGAGGCAGCCAGCGCAGGGGCTTCTGCTGAGGGGGCAGGCGGAGCTTGAGGAA
ACCGCAGATAAGTTTTTTTCTCTTTGAAAGATAGAGATTAATACAACTACTTAAAAAATATAGTCA
ATAGGTTACTAAAGATATTGCTTAGCGTTAAGTTTTTAACCGTAATTTTAATAGCTTAAGATTTTAA
GGAGAAAATNTGAAAGACTTTATAAGAGTAGCANTGAGGGAAGGGNAAAGGATAAAAAGGTTTN
TAAAAACATGAACGGGAGGGTTGAGGANGAAAGCCTTCTTCATGGGAGTNAAAAAAAAATGTTNT
TTNAAAAA

| | |
|---|---|
| Sequence 1368 | cMhvSA018f11 |

NCCTTAGCGTGGTCGNGGCCGAGGTACAGACAGGCAGGCTCCCAGTGTGAGAAGTGCCTTTAGGA
CAAGTAGAACTGCACACATAGATGCAAATGCCTGGGCCTTTCTTCAGGTTCTGTCATAGAACANAC
TGCCTGAGGCCATGCTCANGACTGCNGGCCTCAGAAACCCAGCACTTGCCCCTGCTCTGTCTTTCT
GCTCCCAGCAGCTGAATTCTAGGGAAATGTCTNTCCNTCANCCCACCCCGAGACAAACCTGCCAA
GCTNNTGGCTNTCAAATNCTTTTGCCCATGACTGANGTCCCATCANCCCTTTTCCCCAATATGAGA
ATAGCTTGTTCCACCCCTCCAAGTNCAGCAAGGCATGGGGATAACTGGAAAGGCTGTTACACCTGT
ATGCTCTCCTGCTCCCTAAGCCTGCCTCAAAACATG

| | |
|---|---|
| Sequence 1369 | cMhvSA019a04 |

CCCTTTCGAGCGGCCGCCCGGGCAGGTACCAACAGAAACAGAAATAACTGAGCAACCGAACCACC
AATAGAGCTCTTAGATTAAGAACCTTGGTTCAAGGAAGGAGTTTTGAGCAGGTGCTGGACAGAAA
GACTGAGAACTCTATGATGTAAATGAGAGCCCTGTGATAAGCCAATCAGCCTGCTGTGGCCTGGA
ACTGATTGATCATGGGCCAGGAAGGAGCACAGAGGGGTAACCTGGCAAAGAACAAAGGAAGAGG
TAGCCACTGGCGGAGAATGACTAGGACAGAAGANGCCCAGAAGAGAGCTAGGACTGGGAATCAA
ATTTACATATGGATGTCTAAGAAAACTTTANGTTCACAATGAGGCTTCTTNTTANGCATAACCTGC
AGATGATCAAGAATGCTTTTTTTTGCTTGGTTGGNTTCTAAAT

| | |
|---|---|
| Sequence 1370 | cMhvSA019d08 |

CCCTTTCGAGCGGCCGCCCGGGCAGGTACACTCTTTCTTGGTCATGTGGCTTCCCTGTTTCTTCACA
ATTGCAGCTACATTCCCTCTCAATGCTCTGAAAGTGTGGGTGCCTCTCCCCCTTTAGTTCTGGCTGT
AGACAGTGGTTTGGCACTCCTSGGCTGTCTACTGCAGCTCTGGGTGATCAATCTAATGTTTATGTTC
CTTCCCCAGCTTGTTTGCAGCAGAGGAAGGAACCTTAGTAGTGGTCATGGCCAANGGTCCCTTGCT
CATCTCCTGGGGACTCCACTCTAGAGATACACAGGTCAGCAATTGTTTTGGTGCAATCAAGCCTAG
GGATGGAGGGTCTGTNCTGTGGGCCCAAACCAAGGGGGTCCCTGTCTGATGATNAANCAATGGAA
GGGTTGTTGTGGNAACCACATTNGGNANAGGGGACNTGGCCTTCTTTCTCCCTTGGGGNTTGAATT
GCANCCCNTGTTTGGAAAGTGGTGGGATNAAAANGCACCGTTGGGGGNCTTTTGATTCTTTTNGNT
AANNCCCTGNAANGGGTAANCCAAAANAACNANTTNTACTTGCAAAAANGCAATTGGGGCANA
AAAAAGGGTTTTT

| | |
|---|---|
| Sequence 1371 | cMhvSA021g07 |

CCCTTAGCGTGGTCGCGGCCCGANGTACTNTTTTTTTTTTTTTTTTTTTTTGTCTGGGTGGTGACAGC
TCATGATAATTCATAAAGTTGTATACTATGATTTGTGCATATTGGATANATACGTCATAGTTCACTT
TAAAAGTTT

| | |
|---|---|
| Sequence 1372 | cMhvSA023d02 |

CCCTTTCGAGCGGCCGCCCGGGCAGGTACAATTCCAGGAGCTTCCCTGTAATTCCTCAAAAAAGCA
CTAGTAAAACTCTTAGGAGGATATTAGATAAAGCTCACTTAGCAATAGCCCTTTTTCCCCACATAT
TCTGGAAGGTTCTATAAAAGCTATTAGATACTCATTCCTGGTTCTGGAAAATTAAATAAGCCAATT
CTTGGTAGGATTTTCCAAANGGCTTACCACAGGAGGGATTTTATNCCTCNTTTTTGAAAAATATTTT
CATCCCATTAANAGNAATAAGGAAANCTTCTTGCCCTTTCAATAAGCCATTTTTNANAGGCCTTTC
CTGGTTATTTTTNNTTGGGGGACCAAAAAAAAATTNGTTCTTANAAACCAAGNAAANTTTAAGAAT
TCTTTCCCAGGGGTCCTTCAAAAAAAGGCCACCAAAGGANGNANTATTTATTCCAANGGAGGAAA
AAATTCTTTGGGAAGNTTAAAAACCNCAAAAACCAAAAAAAATTCTTGNTANAAAAATGGTGGGN
GAAAAATTGGTACAATTTCTTCCCTTTTCC

| | |
|---|---|
| Sequence 1373 | cMhvSA023h11 |

CCCTTTCGAGCGGCCGCCCGGGCAGGGACTTATTTATATTTTATTTNTNNCATTGNNTNTTTAAGGN
TTGNNATTGNANTNANTTTNNAANTNAATTNTAACTGTTTNCNGNTTTTTCAATGTGTTTATNTANT
NCATCNGATTTTGNACTNANCGAGCCTNCACAATTATGTCAAAAAGCTAATATGTTTGAGAACCAT
CTATTTAAAGAACAGCAAGTTTGGACCAANAAATAAAGACCAACGGTGAAAGCANGCAANCCCC
AGAAATAACTAGNAAAANTGCTNAAAAGGAGGAACCTTTTACTTNATANGANAATNAAACCATTT
GACNGNAAAACTTTTTTNAACACTAANATTTNTATNTTTTTNAAANNNACCTTTTTTTTT

| | |
|---|---|
| Sequence 1374 | cMhvSA024h04 |

CCCTTAGCGTGGTCGCGGCCGAGGTACTTTTTNTCTTTCTTTTTTCTTTTTTTTTTTTTAACAGGAAT
CAAGTNAAAACCACAGAACCTCTATATTTATATTTGAGTCTGAATCAAACATTTTCACTTGGAAGA
ATTTTTTCCAAAGGGGGGGAAAACAACTGNTTCTGAGTGCCTTTATTTTAGGTTAATTTTTTCAAAA
GATTATCTCTGACACCTTTGCATTAAGTATCTAATGTATTACGTGGGACTCCATGGGCTGCATTTAT
TTCTTCAATTTGCTAAAAAAAAAAAAAAGNCTACTAAAATTTCAATTTTTGAAAAGCAATTAATNT
GAAATATNTTAGATAAGCNAAAATGTAATAAACTCTTNCACTATTTTTTTG

| | |
|---|---|
| Sequence 1375 | cMhvSA027g09 |

ACCGNGCCTGCCTNTCAAGATACCCCATCCTCTCCACGCCGCTGCCGCTGCCGCCATGCAAGGGGA
GGACNCCNGATACCTCAANAGGTGACGACTCCCCAACGGCTCTGTCCTACCCTCCTTGCCAGGGCC
CTGAAGATGNTCTTGGGTTTGCTGNGAGATGTCACNTGGGCAAACGCTTAGCTTATTCACTACGGG
ATGGGGAAAGCNNGGAGAGTAAGTTCACTCGGAATAGGGAGGAGGGGAAAAGGTGAANATGGGN
CAAAAAAANGAGNAGCNTNTGGGGGGGTTTTNAAAAGTCCCTTTGACCTTGAACTCGGCGNNATC
CCCNTTTCAGCCTTTGANAAAGATNGGGGTTCCTTTCCGCTTANCANTCAACCCTTTAATTNANCA
AGNGNGNGAAGAAGGGGAAGGNTTANNTGGCCAANNGGTAAAAACCCCCCCGCNCCTTTTTGNTT
TTT

| | |
|---|---|
| Sequence 1376 | cMhvSA031d05 |

CCCTTAGCGTGGTCGCGGCCGAGGTACTTTTTTTTTTTTTTTTTTTTTNNNCCCCTTTTTTTTTTTTTT
TTNCNNNAANNTNNTTNAAAAANNAAAAANNNNTTTNCCCAAAAAAANNAANNCCCNGGGGAAA
NGGGGCCNGGGGGNANAANTTAAAAAANANTTTNAANCCCNNCCCCCNNTTTNNNTTTAAAAATN
TTNNANNGGGGGCNNNCCNTNTCANACCTTGCNNCTGGGNTTATNAATTTACTGCCNTTCCATTGT
ATTGAGGTCCCTGAANTCNTGGATNACCAGAAANGGGGANTTTTAANATTNCATTNAAT

| | |
|---|---|
| Sequence 1377 | cMhvSA033f08 |

ACTTCATGAACGCCAGGAAAGCCTTCAGGCTATCCTCAACAGAATGGAGGAGGTTCACAAGGAGG
CAAACTCTGTGCTGCAGTGGCTGGAATCAAAAGAGGAAGTCCTGAAATCCATGGATGCCATGTCA
TCTCCAACCAAGGACAGAAACAGTGAAAGCCCAAGCTGAATCTAACAAGGCCTTCCTGGCTGAGT
TGGAACAGAATTCTCCCAAAAATTCAAAAAAGTTAAAAGGAAGCCCTGGCTGGATTTACTGGTGG
ACATATCCCAACTCACAGGGAAAAAAGNATTANAATGCTTTNTGGTTACCTTGGCCCGCGGACCC
ACCGNCTAAGGGGCGAAATTCCAGCACACTGNGCGGCCCGTTTACTAGTGGGATCCCGAGGCTCG
GTTCCAAANNCTTTGGGCCGTAAATCANTGGNTCANTAGCTTGTTTTNCTGNTGGNGAAAAATTNG
TTTATTCNCCGCTTCACCAAATTTCCCCCACCAACCATTAACCGANGCCCCGGGNAAAGGCCATTA
AAAANGTGGTTAAAAGCCCCTGGG

| | |
|---|---|
| Sequence 1378 | cMhvSA034a02 |

CCCTTTCGAGCGGCCGCCCGGGCAGGTACATTGAAGCTGCTTAAATAACCCAGTATCTGAAAAGCT
GTCCTCTTAACATTGCATTAATAACAATATAAGCTCAATTTTAAATGATGAAATATTTCACCCTCCC
TAGTTTCTGATTTTGGCCTCTGGAGTAATNTTAACTTGATCAGTAAACACACACATTACATACATAC
ATTATTACACACACCAAAGGTTTCATTCATTATTTAAGCAAGGAGAATCGGATTACCCCTTGTGTT
AATTNATNATTAAGGAAAANTTCCAAAAAAAGGTCNAAACCTCCAGTTAGGCNATGNCTTAATGG
AAAANTAANCTAAGTNATTTCAAANAATCCAAAAAGGGTGGGAAAAAATTTCAAGCCCANCTTGG
GGGGGNACCCCTTGAAAAAGGGGTTTCCCTTCACTTTTCCCCTTAAGNAAATTATTATTAACCATTT
TTGGGAAA

| | |
|---|---|
| Sequence 1379 | cMhvSA034b07 |

CCCTTAGCGTGGTCGCGGCCGAGGTACTGCTCGGAGGTTGGGTTCTGCTCCGAGGTCGCCCCAACC
GAAATTTTTAATGCAGGTTTGGTAGTTTANGACCTGTGGGTTTGTTAGGTACGCGGGGGGAGTCTN
CAGGATGGCACCGGACCCCTGGTTCTCCACATACGATTCTACTTGTCAAATTGCCCAAGAAATTGC
TGAGAAAATTCAACAACGAAATCAATATGAACGAAAAGGTGAAAAGGCACCAAAGCTTACCGTG
ACAATCAGAGCTTTGTTGCAGAACCTGAAGGAAAAGATCGCCCTTTTGAAGGACTTATTGNTAAG
AGCTGTGTCAACACATCAGATAACACAGCTTGAAGGGGGACCGAAAACAGAACCTCTTTGGATGA
TCTTGTANCTCGAGAGAGACTACTTTCTGGCCATTCTTTAAGAATGAGGGTGCCGAACCAGATCTA
ATCAGGTNCAGCCTGATTAGTNGAAGAGGCTAAACNAGNAGNANCNAAACCCTTGGCTTTTTTTA
GGGNGCCCNCNGGAAGACCNAGAAGGCTTTGGGTTTTGATTAAAATNCGGGCAACAAGNAGGCA
GAAAAAATTNTTCNAAANAACAAGGATGCCAAGCCCTTTGATNCCCCTTTTCCTTTTATNNAAAAA
NGTTGGCCANAAAANAAAATTGGGGGGCAAGGNAAATTTGGGGAATTTNAATTTGGGATTNAACC
AAAAATGAGNANTAANTTTNGNCCNNCCTTNCCCAACCTTTTGGGNNNAAAACANAATTNAAAAA
ATTTT

| | |
|---|---|
| Sequence 1380 | cMhvSA041e12 |

CCCTTAGCGTGGTCGCGGCCGAGGTACTTTTTTTTTTTTTTTTTTTTTTTTTANNGGGNTNNNNNNTT
TTCCCCNGGGNNGTTTNAAANNNNNGGGCNNAAANAAANNCCCNAANNAANCCNTTAAAANGNN
GGGNANNANNGGNNAAAANNAANNCNAANGGCCCNANNNNNTTTTTTTTNAAACCAAAAANNNT
TTAAAAAAAAAAAANNTTTTTTTTTTAANNAAAANTAAANCCCNGAANGGGNTNCCTTTTTNCCCC
CGGGGNNNNGAAAAAAAACNCCTTANNNCCNTTNANACCCNGTTTTCCCTTNGCCCCCCCAAATT
TCAAAAN

| | |
|---|---|
| Sequence 1381 | cMhvSA045f05 |

CCCTTTCGAGCGGCCGCCCGGGCAGGTACTTTTTTTTTTTTTTTTTTTTTTTTATTTNCAATGCTTCGT
TTCTAGCTATTCTGTGCTCATTTCCACCTGAAAGANAAAATAATACTATCTATAGCTGAGATTCATA
TTATGGAATAGTAATTTATTCTATATCTGTAACTTTTAAAAAGTCATAATTACATCAATGCACATGT
AAGTTAAGGGAGTTATTTGTTTTTCAAAGAAGGCGTCCACAGTTCGACTTTAAATAAGTTGNGTAG
GAACACTACATCTGTTCTCAAGGGATTCCACCAAATACTTTTTGGTGCTTCCTTTAAAACTGCCACC
AGAGCCACTTTACAAGGTATAAACAGGGTTTGGGAGGCCCTATATTATACCTCATTTTCACCCAAA
CGTATTGCCCTTTGCATTTT

| | |
|---|---|
| Sequence 1382 | cMhvSA045h03 |

CAATTTTGGAAAATTCCCGCAAGNTAAANCGNTTTTCAGGGAGATTTATNTCGNTTTAATACCCCC
NTAGCGAGGNCGNGGGCGATGTACAANAACTANNTGGTTGTGGTGGCGCTCGCCTGTAGNCCCAG
CTACTCGGGAGGCTGAGGCAGGATAATTGCTTGAACCTGGCANGCAGAGGTTGCAGTGAGCCGAG
ATCGCAGTCACTGCACTCCAGCCTGGCGACAGAGCGAGACTNCGCCTNGGGAAAAAAAAAAAAA
ATCCTTAACAGCTGAGAATGGCTAGAGTTTAGGCGCTGCACACTGGCAAGCAGCTCCTTTGACCCC
AGGCACTTNACTCCTCATTTNTCTCTNAACAAGGCAGCCAGCAAGGATCCTGGAGTCACAGGGTGT
GAGATGCGAAAAAA

| | |
|---|---|
| Sequence 1383 | cMhvSA051e01 |

AGGTACCAACTGGGACCGTTGAAACTGTTTAGCCTTTGTGGCAAGAAATTCCGATTTCATTTCAAC
TCCTGCTTGTTGTAGAATTGACTTTGCCACAGGTCCAACTGTAATATCATGTGGGTTTACAGAATTA
ACAATTACATCTGCCGTCTGCCATTCAATGTGGCCCTGGACAATCTGGAGGGTCAGGTTGTTCACG
ACCATTGCATTGAAAGAAGGGGTGGTTTCTTGTCCCAGCTCACTCTTCCCTAGGATGAATTCTGAA
GCAGCTTTAAAGGCAGCAACAGTAGGGTCCTCATTGCTCACCAGGTGAATTTCTTTCAAATTACTC
ATCATTGGCTTCCTTGCAAACTAACCCGGATAGTCTCTACAATAGTCTTTGTACCTGCCCGG

| | |
|---|---|
| Sequence 1384 | cMhvSA052c03 |

NCCCTTTCNAGCGGCCGNCCGGGCAGGNNCANNTTCACTCACATGTGGCTCTNGGNTGTATTCNGN
AGNGGGCATCNTGACCCACATGATCAAATGCCCCAGAGTTCACTCTNTNTNTGAAGAGCTCCGTGT
CTACTAAGAGGTCTGATTCCCTACATGCNGGCCAGTATGTNGGAATGAAATGTGTCACTAANCGTN
AAAATAANGCACTAGCAAATNCAGAACCTTGAAAAGTNAAACTNATNCCNNCCAAGGGCTTNATT
TTTCAGGGGCC

| | |
|---|---|
| Sequence 1385 | cMhvSA055c11 |

CCCTTTCGAGCGGCCGCCCGGGCAGGTACGCGGGGAGGCATTGAGGCAGTCAGCGCAGGGGCTTC
TGCTGAGGGGGCAGGCGGAGCTTGAGGAAACCGCAGATAAGTTTTTTTCTCTTTGAAAGATAGAG
ATTAATACAACTACTTAAAAAATATAGTCAATAGGTTACTAAGATATTGCTTAGCGTTAAGTTTTT
AACGTAATTTTAATAGCTTAAGATTTTAAGAGAAAATATGAAGACTTAGAAGAGTAGCATGAGGA
AAAAAAAAAAAAAAAAAAAAAGGTACCTCGGCCGCGACCACGCTAAGGG

| | |
|---|---|
| Sequence 1386 | cMhvSA055c11 |

CGCTCACAATTCCCACACAACATACCGAAGCCGGAAGCATTAAAGTGTAAAAGCCTG

| | |
|---|---|
| Sequence 1387 | cMhvSA055d08 |

CCCTTACCAGCGGCCGGNCNGACNGNCACTTNNNNNCACTGNNGGGGNCCATTGTNACTGNCANG
GAATACTTGAAAGGTCANGTAACTNACACTTCTGGAGAGACCATTCAAGGCTTGTGNCTNTTGACA
AAAANAGACCANTNGNGCAATGAAAAGGAGAGAATTCT

| | |
|---|---|
| Sequence 1388 | cMhvSA001e01 |

CCCTTAGCGTGGTCGCGGCCGAGGTACACAGAACTTGAAATTTGCAAAAGAAGGAGA

| | |
|---|---|
| Sequence 1389 | cMhvSA002a05 |

CCCTTTCGAGCGGCCCGCCCGGGCAGGTGCTTTTTTTTTTTTTTTT

| | |
|---|---|
| Sequence 1390 | cMhvSA002f10 |

TTCCCTTAGCGTGGTCGCNGCCGACGTACACNTGGACCTGCTGGCATTCGAGGNCCTCANGGTCAC
NAAGGCCCTGCTGGCCCCCC

| | |
|---|---|
| Sequence 1391 | cMhvSA004c04 |

ANAATTCGCCCTTAGCGTGGTCGCGGC

| | |
|---|---|
| Sequence 1392 | cMhvSA009h05 |

ACATTCATGTTAATCCAGGGAGCAAGGTAAAGCTGTCACTTTCATTATTCACATGACCACGAAAAT
AAATTGTATTTTTTTTTTTT

| | |
|---|---|
| Sequence 1393 | cMhvSA013c02 |

CCCTTACCAGCGGCCGNNCCGACNGNCNCAATTACTNCTATTTNNAATNTACNAAGGANCAAACA
NCTACAGGATTNAGGNCGGACCGAATGGGT

| | |
|---|---|
| Sequence 1394 | cMhvSA014d07 |

AGCGTGGTCGCGGCCGAGGTNCATNCTAACAAANATGAAATNCTATGTTAAATCTACTAACNCTTT
GCCTGCCA

| | |
|---|---|
| Sequence 1395 | cMhvSA019b03 |

CCCTTTCGAGCGGCCGCCCGGGCAGGTACTTTTTTTTTTTTTTTTTTTTTTTTTACGGNTTTTATTTTT
NAATTTTTATTTTGGTTTTCTTACAAAGGNNGACATTTTCCATAACAGGTGTAAGAGTGTTGAAAA
AAAAAATTCAAATTTT

| | |
|---|---|
| Sequence 1396 | cMhvSA021h04 |

GGTATGCTTGACCNTAGNGCTANCATCTTCTTTACAATTTNNANAAGGCAGAGGATGAAGACNAA
CCAAGAGGCTACTGNCATTGAATTT

| | |
|---|---|
| Sequence 1397 | cMhvSA023d09 |

AGGGAGGAAAGGGANAAANANATGACAANAGCAAGACACAAGAAATGCAGCAATAAGCACACA
NNACTCACACACTGACNCTAATCTGGNGCAGGCCATCCTCTTAC

| | |
|---|---|
| Sequence 1398 | cMhvSA026c06 |

CCCTTCGAGCGGCCGCCCGGGCAGGTACTTTCTTTTTTTTTTTTTTTTTTTTTTATTTTTTTTTTTTTTT
TTTTTTTTTTTTTTTTTTTTTTTTTTTTTTTTTTTTTTTTTTTTTTTTTTTTTTTTGGGGNAAAAAGGGTT
TTTNTTTTCCCCCNNNTTCCNNCNTTTTATTTTTTTT

| | |
|---|---|
| Sequence 1399 | cMhvSA031f12 |

CCCTTTCGAGCGGCCGCCCGGGCAGGTACTTTTTTTTTTTTTTTTTTTTTTTTTTTTCCCNTTAAAAA
AANNACNTCCAATNGNNNTCAACCNNGGGNAAAAAAGGGGNNGGGGGTNTTTTAAGGGGAAAAA
NNAAAAAAAAAAAAAGGGTTTTTT

| | |
|---|---|
| Sequence 1400 | cMhvSA032c04 |

CCCTTTCGAGCGGCCGCCCGGGCAGGTACATGTGCATGTTTTTACATGGGTATATGGCATACTGGC
GGGGACTGGGCTTCTAGTGTATCTATTCCCCAGCTAGTGAACATTGAACCTATAGGTAATTTTTCA
ACCCTTGCCCCCCTCCCCACTCTCCTCGCTTTTGGCATTCCCAGTATCTATTATAAGGCTTGGGTTTT
AATATACCTGCTTCTGCACTGAGTCTGTGGACCAGGGTACCTCGGCCGCGACCACGCTAAGGG

| | |
|---|---|
| Sequence 1401 | cMhvSA032f08 |

ACTTTTTTTTTTTTTTTTTTTTTTAAANCCNNNAAAAAAAAAAAAANTTTCCCNATTTTTTTNNNAG
GGGTTTTNTGGNNANNGGGNAANNNGGGGGNTTNGGNNNNNNNAANNNTTNNNNCCCNNNNTTT
NAANTTNCCCNGGNNNAAANAANGNAACCCCCNNNTTTNAAANNAAAAAAAAAANG

| | |
|---|---|
| Sequence 1402 | cMhvSA032h03 |

CCCTTAGCGTGGTCGCGGCCGAGGTACTTTTTTTTTTTTTTTTTTTTNGCCNNAAANGGGGGNAANG
GGGGNNNTNNNGGGAAAAANCNGCCCCTTTTTAAAAA

| | |
|---|---|
| Sequence 1403 | cMhvSA033g03 |

CCCTTTCGAGCGGCCGCCCGGGCAGGTACTTTTTTTTTTTTTTTTTTTTTTTTGANNNCCNNCCNNNA
ANNAAAANAANNNTTTTGGGNCNNAANTTTTTTNNNNNTTAAAAAAAAANAAACNNAAANTTTN
AAAAANANNCCNNTNTNTTTTTTTTTNNGGGGGGNNNAAAAAAAAAAA

| | |
|---|---|
| Sequence 1404 | cMhvSA044b10 |

ACCCTTGCCTTTGAATNATTTATATNCTNATNTTTCTTGNNCCCAGACTTTGTCCTTCANTGCACTG
AGTCAAAGCTTTACACTA

Sequence 1405 cMhvSA048f10

CCCTTTCGAGCGGCCGCCCGGGCAGGTACTTTTTTTTTTTTTTTTTTTTTTTTTTGGTTTTTTTTTTTTT
TTTTTTTTTTTTTTTTTTTTTTTTTTTTTTTTTTTTTT

Sequence 1406 cMhvSA007h11

CCCTTTCGAGCGGCCGCCCGGGCAGGCACTTTTTTTTTTTTTTTTT

Sequence 1407 cMhvSA009d02

CCCTTTCGAGCGGCCGCCCGGGCAGGTACTTTTTTTTTTTTTTTTTTT

Sequence 1408 cMhvSA010e01

CCCTTTCGAGCGGCCGCCCGGGCAGGTACTTTTTTTTTTTTTTTTTT

Sequence 1409 cMhvSA018b01

CCCTTTCGAGCGGCCGCCCGGGCAGGTACTTAGTTTTTTTTTTTTTTT

Sequence 1410 cMhvSA032f11

CCCTTTCGAGCGGCCGCCCGGGCAGGTACTTATATTTTTCTTTTTTTTTTTTTTTTTG

Sequence 1411 cMhvSA037a06

CCCTTTCGAGCGGCCGCCCGGGCAGGTACTTTTTTTTTTTTTTTTTTTTTTTTTTTT

Sequence 1412 cMhvSA037d04

CCCTTTCGAGCGGCCGCCCGGGCAGGTACTTTTTTTTTTTTTTTTTTTTTT

Sequence 1413 cMhvSA037f01

CCCTTAGCGTGGTCGCGGCCGAGGTACTTTTTTTTTTTTTTT

Sequence 1414 cMhvSA040g04

CCCTTAGCGTGGTCGCGGCCGAGGTACCTTTTTTTGTTTTCCTTTTTTT

Sequence 1415 cMhvSA041d06

CCCTTAGCGTGGTCGCGGCCGAGGTACTTTTTTTTTTTTTTTTTTT

Sequence 1416 cMhvSA041f04

CCCTTTCNAGCGGCCGCCCGGGCAGGTACTTTTTTTTTTTTTT

Sequence 1417 cMhvSA037b05

CCCTTAGCGTGGTCGCGGCCGAGGTACTTTTTTTTTTTTTTTTTTTNGGGNAAAAAAAATTTNNNTT
TTTNCCCCNNGNNNGNNNGNNNNGGGGCCNTNNAAATTTNNTNGNNCCCCCCCCCCCCCNTTTAA
AAAAATTTTNNNNCCNTANCCCCCCAAATTATNNGGNTTAAAAGGNTTTGCCNNNNTCCCNGGGN
NNTTTTTTTTTTTTA

What is claimed is:

1. A method of assessing whether a patient is afflicted with breast cancer, the method comprising comparing:
a) the level of expression of one or several breast cancer marker genes in a patient sample, and
b) the normal level of expression of one or several of said marker genes in a sample from a control subject not afflicted with breast cancer,
wherein at least one of said marker genes is selected from the group consisting of the genes listed in Table 1 and a significant difference between the level of expression of one or several of said marker genes in the patient sample and the normal level of one or several of said marker genes is an indication that the patient is afflicted with breast cancer.

2. The method of claim 1, wherein several of said breast cancer marker gene is selected from the group consisting of the genes listed in Table 1.

3. The method of claim 1, wherein at least of one of said marker genes encodes a secreted protein.

4. The method of claim 1, wherein the sample comprises cells obtained from the patient.

5. The method of claim 4, wherein the sample is a breast tissue sample.

6. The method of claim 5, wherein the cells are in a fluid selected from the group consisting of blood fluids, breast fluid, lymph fluid and urine.

7. The method of claim 1, wherein the level of expression of said marker genes in the samples is assessed by detecting the presence in the samples of a protein encoded by each of said marker gene or a polypeptide or protein fragment comprising said protein.

8. The method of claim 7, wherein the presence of said protein, polypeptide or protein fragment is detected using a reagent which specifically binds with said protein, polypeptide or protein fragment.

9. The method of claim 8, wherein the reagent is selected from the group consisting of an antibody, an antibody derivative, and an antibody fragment.

10. The method of claim 1, wherein the level of expression of said marker genes in the sample is assessed by detecting the presence in the sample of a transcribed polynucleotide encoded by each of said marker genes or a portion of said transcribed polynucleotide.

11. The method of claim 10, wherein the transcribed polynucleotide is an mRNA or hnRNA.

12. The method of claim 10, wherein the transcribed polynucleotide is a cDNA.

13. The method of claim 10, wherein the step of detecting further comprises amplifying the transcribed polynucleotide.

14. The method of claim 1, wherein the level of expression of said marker genes in the samples is assessed by detecting the presence in the samples of a transcribed polynucleotide which anneals with each of said marker genes or anneals with a portion of said transcribed polynucleotide, under stringent hybridization conditions.

15. The method of claim 1, wherein said significant difference comprises an at least two fold difference between the level of expression of one of said marker genes in the patient sample and the normal level of expression of the same marker gene in the sample from the control subject.

16. The method of claim 15, wherein said significant difference comprises an at least five fold difference between the level of expression of one of said marker genes in the patient sample and the normal level of expression of the same marker gene in the sample from the control subject

17. The method of claim 1, comprising comparing:
a) the level of expression in the patient sample of each of a plurality of marker genes independently selected from the genes listed in Table 1, and
b) the normal level of expression of each of the plurality of marker genes in the sample obtained from the control subject,
wherein the level of expression of at least one of the marker genes is significantly altered, relative to the corresponding normal level of expression of the marker genes, is an indication that the patient is afflicted with breast cancer.

18. The method of claim 17, wherein the level of expression of each of the marker genes is significantly altered, relative to the corresponding normal levels of expression of the marker genes, is an indication that the patient is afflicted with breast cancer.

19. The method of claim 18, wherein the plurality comprises at least three of the marker genes.

20. The method of claim 19, wherein the plurality comprises at least five of the marker genes.

21. A method for monitoring the progression of breast cancer in a patient, the method comprising:
a) detecting in a patient sample at a first point in time the expression of one or several breast cancer marker genes;
b) repeating step a) at a subsequent point in time; and
c) comparing the level of expression of said marker genes detected in steps a) and b), and therefrom monitoring the progression of breast cancer;
wherein at least of said marker gene is selected from the group consisting of the genes listed in Table 1.

22. The method of claim 20, wherein at least one of said marker gene encodes a secreted protein.

23. The method of claim 20, wherein the sample comprises cells obtained from the patient.

24. The method of claim 20, wherein the patient sample is a breast tissue sample.

25. The method of claim 20, wherein between the first point in time and the subsequent point in time, the patient has undergone surgery to remove breast tissue.

26. A method of assessing the efficacy of a test compound for inhibiting breast cancer in a patient, the method comprising comparing:
a) expression of one or several breast cancer marker gene in a first sample obtained from the patient and exposed to the test compound; and
b) expression of one or several of said marker genes in a second sample obtained from the patient, wherein the second sample is not exposed to the test compound,
wherein at least one of said marker genes is selected from the group consisting of the genes listed in Table 1, and a significantly lower level of expression of one of said marker genes in the first sample, relative to the second sample, is an indication that the test compound is efficacious for inhibiting breast cancer in the patient.

27. The method of claim 26, wherein the first and second samples are portions of a single sample obtained from the patient.

28. The method of claim 26, wherein the first and second samples are portions of pooled samples obtained from the patient.

29. A method of assessing the efficacy of a therapy for inhibiting breast cancer in a patient, the method comprising comparing:
a) expression of one or several breast cancer marker genes in the first sample obtained from the patient prior to providing at least a portion of the therapy to the patient, and
b) expression of one or several of said marker genes in a second sample obtained from the patient following provision of the portion of the therapy,
wherein at least one of said marker genes is selected from the group consisting of the genes listed in Table 1, and a significantly lower level of expression of one of said marker genes in the second sample, relative to the first sample, is an indication that the therapy is efficacious for inhibiting breast cancer in the patient.

30. A method of selecting a composition for inhibiting breast cancer in a patient, the method comprising:
a) obtaining a sample comprising cancer cells from the patient;
b) separately exposing aliquots of the sample in the presence of a plurality of test compositions;
c) comparing expression of one or several breast cancer marker genes in each of the aliquots; and
d) selecting one of the test compositions which alters the level of expression of one or several of the marker genes in the aliquot containing that test composition, relative to other test compositions;
wherein at least one of said marker gene is selected from the group consisting of the genes listed in Table 1.

31. A method of inhibiting breast cancer in a patient, the method comprising:
a) obtaining a sample comprising cancer cells from the patient;
b) separately maintaining aliquots of the sample in the presence of a plurality of test compositions;
c) comparing expression of one or several breast cancer marker genes in each of the aliquots; and
d) administering to the patient at least one of the test compositions which alters the level of expression of one or several of said marker genes in the aliquot containing that test composition, relative to other test compositions,
wherein at least one of said marker genes is selected from the group consisting of the genes listed in Table 1.

32. A kit for assessing whether a patient is afflicted with breast cancer, the kit comprising reagents for assessing expression of one or several breast cancer marker genes, wherein at least one of said marker genes is selected from the group consisting of the genes listed in Table 1.

33. A kit for assessing the presence of breast cancer cells, the kit comprising a nucleic acid probe which specifically binds with a transcribed polynucleotide encoded by a marker gene selected from the group consisting of the marker genes listed in Table 1.

34. A kit for assessing the suitability of each of a plurality of compounds for inhibiting breast cancer in a patient, the kit comprising:
a) the plurality of compounds; and
b) a reagent for assessing expression of one or several breast cancer marker genes, wherein at least one of said marker genes is selected from the group consisting of the genes listed in Table 1.

35. A method of making an isolated hybridoma which produces an antibody useful for assessing whether a patient is afflicted with breast cancer, the method comprising:
immunizing a mammal using a composition comprising a protein encoded by a gene listed in Table 1 or a polypeptide or protein fragment of said protein;
isolating splenocytes from the immunized mammal;
fusing the isolated splenocytes with an immortalized cell line to form hybridomas; and
screening individual hybridomas for production of an antibody which specifically binds with said protein, polypeptide or protein fragment to isolate the hybridoma.

36. An antibody produced by a hybridoma made by the method of claim 35.

37. A kit for assessing the presence of human breast cancer cells, the kit comprising an antibody, wherein the antibody specifically binds with a protein encoded by a gene listed in Table 1 or a polypeptide or protein fragment of said protein.

38. A method of assessing the breast cell carcinogenic potential of a test compound, the method comprising:
a) maintaining separate aliquots of breast cells in the presence and absence of the test compound; and
b) comparing expression of one or several breast cancer marker gene in each of the aliquots,
wherein at least one of said marker genes is selected from the group consisting of the genes listed in Table 1, and a significantly altered level of expression of one or several marker genes in the aliquot maintained in the presence of the test compound, relative to the aliquot maintained in the absence of the test compound, is an indication that the test compound possesses human breast cell carcinogenic potential.

39. A kit for assessing the breast cell carcinogenic potential of a test compound, the kit comprising breast cells and a reagent for assessing expression of a gene listed in Table 1.

40. A method for determining whether breast cancer has metastasized in a patient, the method comprising comparing:
a) the level of expression of one or several breast cancer marker genes in a patient sample, and
b) the normal level or non-metastatic level of expression of one or several of said marker genes in a control sample
wherein at least one of said marker genes is selected from the group consisting of the genes listed in Table 1, and a significant difference between the level of expression of one or several of said marker genes in the patient sample and the normal level or non-metastatic level is an indication that the breast cancer has mestastasized.

41. The method of claim 40, wherein several of said marker genes are selected from the genes listed in Table 1.

42. The method of claim 40, wherein at least one of said marker genes encodes a secreted protein.

43. The method of claim 40, wherein the sample comprises cells obtained from the patient.

44. The method of claim 40, wherein the patient sample is a breast tissue sample.

45. A method for assessing the aggressiveness or indolence of breast cancer comprising comparing:
a) the level of expression of one or several breast cancer marker gene in a sample, and
b) the normal level of expression of one or several of said marker genes in a control sample,
wherein at least one of said marker genes is selected from the marker genes of Table 1, and a significant difference between the level of expression of one or several of said marker gene in the sample and the normal level is an indication that the cancer is aggressive or indolent.

46. The method of claim 45, wherein several of said marker genes are selected from the group consisting of the marker genes listed in Table 1.

47. The method of claim 45, wherein at least one of said marker genes encodes a secreted protein.

48. The method of claim 45, wherein the sample comprises cells obtained from the patient.

49. The method of claim 45, wherein the patient sample is a breast tissue sample.

50. An isolated nucleic acid molecule comprising a nucleotide sequence of Table 1.

51. A vector which contains the nucleic acid molecule of claim 50.

52. A host cell which contains the nucleic acid molecule of claim 50.

53. An isolated polypeptide which is encoded by a nucleic acid molecule comprising a nucleotide sequence of Table 1.

54. An antibody which selectively binds to a polypeptide of claim 53.

55. A method for producing a polypeptide comprising culturing the host cell of claim 52 under conditions in which the nucleic acid molecule is expressed.

56. A method for detecting the presence of a polypeptide of claim 52 in a sample comprising:
a) contacting the sample with a compound which selectively binds to the polypeptide; and
b) determining whether the compound binds to the polypeptide in the sample to thereby detect the presence of a polypeptide of claim 53 in the sample.

57. A kit comprising a compound which selectively binds to the polypeptide of claim 53.

58. A method for detecting the presence of a nucleic acid molecule of claim 50 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 45 in the sample.

59. The method of claim 48, wherein the sample comprises mRNA molecules and is contacted with a nucleic acid probe.

60. The method of claim 48, wherein the sample is isolated from ovarian tissue.

61. The method of claim 48, wherein the sample is a tumor sample.

62. A kit comprising a compound which selectively hybridizes to a nucleic acid molecule of claim 50.
